(19) United States
(12) Patent Application Publication
(10) Pub. No.: US 2025/0327116 A1
(43) Pub. Date: Oct. 23, 2025

(54) DETECTION ASSAYS

(71) Applicant: Sherlock Biosciences, Inc., Watertown, MA (US)

(72) Inventors: William Jeremy Blake, Winchester, MA (US); Xiang Li, Malden, MA (US); Mary Katherine Wilson, Burlington, MA (US); Christine Marie Coticchia, Brookline, MA (US); Brendan John Manning, Brighton, MA (US); Pradeep Ramesh, Washington, DC (US)

(21) Appl. No.: 19/080,542

(22) Filed: Mar. 14, 2025

Related U.S. Application Data
(63) Continuation of application No. 17/795,815, filed on Jul. 27, 2022, now abandoned.
(60) Provisional application No. 62/966,527, filed on Jan. 27, 2020.
(60) Provisional application No. 62/967,536, filed on Jan. 29, 2020.
(60) Provisional application No. 62/970,159, filed on Feb. 4, 2020.
(60) Provisional application No. 63/038,710, filed on Jun. 12, 2020.
(60) Provisional application No. 63/139,267, filed on Jan. 19, 2021.

(51) Int. Cl. C12Q 1/683; C12N 9/22; C12N 15/113; C12Q 1/6844

(52) U.S. Cl. CPC C12Q 1/683; C12N 9/22; C12N 15/113; C12Q 1/6844; C12N 2310/20; C12N 2320/10

(57) ABSTRACT

The present disclosure provides improved detection (e.g., diagnostic) assays that utilize a Cas protein collateral cleavage activity.

### CROSS REFERENCE TO RELATED APPLICATIONS

This application claims priority to each of U.S. Provisional Patent Application Nos. 62/966,527; filed Jan. 27, 2020; 62/967,536; filed Jan. 29, 2020; 62/970,159; filed Feb. 4, 2020; 63/038,710; filed Jun. 12, 2020; 63/139,267; filed Jan. 19, 2021 the entire contents of each of which are hereby incorporated by reference.

### SEQUENCE LISTING

The present specification makes reference to a Sequence Listing (submitted electronically as a .xml file named “2013065-0636_SL.xml”). The .xml file was generated on Feb. 21, 2023 and is 614 KB in size. The entire contents of the Sequence Listing are herein incorporated by reference.

### BACKGROUND

A variety of clustered regularly interspaced short palindromic repeats-CRISPR-associated (“Cas”) proteins have been discovered to have a collateral cleavage activity useful in detection (e.g., diagnostic) systems to detect particular nucleic acids of interest. See, for example, review by Sashital Genome Med 2018:10, 32.

### SUMMARY

The present disclosure provides improved detection (e.g., diagnostic) technologies that utilize Cas-protein collateral activity.

Among other things, the present disclosure identifies the source of a problem with use of certain Cas enzymes in certain collateral activity assays. For example, the present disclosure documents that certain such assays include a step that involves incubation at an elevated temperature for a period of time, and various Cas enzymes may be insufficiently stable to maintain a sufficient level of activity (e.g., collateral activity) under such conditions. In many embodiments, such a step may be or comprise a nucleic acid extension and/or amplification step.

Alternatively or additionally, the present disclosure provides the insight that particularly desirable embodiments of various collateral activity assays are those that can be performed in a single reaction vessel (i.e., so-called “one pot”) assays. The present disclosure appreciates that Cas enzymes whose activity (e.g., collateral cleavage activity) is insufficiently stable to maintain sufficient activity through any and all elevated-temperature step(s) (which may be or include, for example, one or more nucleic acid extension and/or amplification step(s)) may not be useful in such one-pot assays. The present disclosure furthermore documents that certain Cas protein(s) (e.g., Cas13 and Cas12) are insufficiently stable at relevant temperature(s), e.g., at temperatures at which nucleic acid extension and/or amplification reactions are typically performed (e.g., above about 60-65° C.).

The present disclosure encompasses the recognition that thermostable variants of various Cas proteins (e.g., Cas9) have already been described and/or otherwise made publicly available (see, for example, Mougiakos et al. Nat Commun. 8:1647, 2017). Those skilled in the art are able to compare such thermostable variants with related non-thermostable homologs (e.g., orthologs), in order to assess sequence changes and/or elements that may be necessary and/or sufficient to achieve thermostability, and furthermore can identify such sequence changes and/or elements in other homologs (e.g., orthologs) and/or can introduce them thereinto. Still further, those skilled in the art are well aware of potential sources of naturally-occurring thermostable Cas proteins (e.g., in microbes that survive in elevated temperature conditions, such as in sea vents, or are otherwise thermophilic). Thus, those skilled in the art, reading the present disclosure, could readily identify and/or develop appropriate thermostable Cas proteins for use as described herein.

In some embodiments, a useful thermostable Cas protein is a Cas12 or Cas13 homolog (e.g., ortholog). In some embodiments, a useful thermostable Cas protein is a Cas enzyme comprising an amino acid sequence having 80%, 85%, 90%, 99% or 100% sequence identity to any one of SEQ ID Nos. 1-283.

Alternatively or additionally, in some embodiments, a useful thermostable Cas protein performs (e.g., its collateral cleavage activity functions sufficiently) at temperatures above about 50° C.; in some embodiments, above a temperature selected from the group consisting of about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C., or combinations thereof. In many embodiments, useful thermostable Cas protein performs (e.g., its collateral cleavage activity functions sufficiently) at temperatures above about 60° C.

In some embodiments, a useful thermostable Cas protein performs (e.g., its collateral cleavage activity functions sufficiently) within a temperature range at which nucleic acid extension and/or amplification reaction(s) are performed; those skilled in the art are well familiar with various such reactions and the temperature ranges at which they are performed, In some embodiments, such a temperature range may be above a temperature selected from the group consisting of about 60° C., about 61° C., about 62° C., about 63° C., about 64° C., 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C., or combinations thereof. In some embodiments, a temperature range may be about 60° C. to about 90° C. In some embodiments, a temperature range may be about 60° C. to about 80° C. In some embodiments, a temperature range may be about 60° C. to about 75° C. In some embodiments, a temperature range may be about 65° C. to about 90° C. In some embodiments, a temperature range may be about 60° C. to about 80° C. In some embodiments, a temperature range may be about 60° C. to about 75° C.

Thus, as is set forth herein, in some embodiments, a useful thermostable Cas protein is a Cas12 or Cas13 homolog (e.g., ortholog), e.g., a Cas enzyme comprising an amino acid sequence having 80%, 85%, 90%, 99% or 100% sequence identity to any one of SEQ ID Nos. 1-283 that is thermostable at temperatures above about 50° C., and in some embodiments above about 60° C., for example within and/or above about 60-65° C. Those skilled in the art, reading the present disclosure will particularly appreciate that, in some embodiments, a useful thermostable Cas protein is a Cas12 (e.g., SEQ ID NO 3-21, 33-47, 51-56, 68-178, and 274-283, or a variant thereof, for example having at least 90%, 95%, 99% or greater amino acid sequence identity thereto) or Cas13 (e.g., SEQ ID NO 1-2, 22-32, 48-50, 57-67, 179-273, or a variant thereof, for example having at least 90%, 95%, 99% or greater amino acid sequence identity thereto) whose activity (e.g., whose target binding and collateral cleavage activities) is sufficiently thermostable, for example at temperatures within a range of 60-65° C. to perform in assays as described herein (e.g., in some embodiments, one-pot assays). For example, in some embodiments, sufficient thermostable activity is activity that is reasonably comparable to (e.g., within about 25%) of an appropriate reference thermostable Cas protein (e.g., SEQ ID NO 15) as described herein.

In some embodiments, the disclosure describes a detection method comprising steps of: contacting a CRISPR-Cas complex comprising: a Cas protein with collateral cleavage activity that is thermostable at temperatures above at least 60-65° C.; and a guide RNA selected or engineered to be complementary to a target sequence; with a sample potentially comprising a nucleic acid of the target sequence.

In some embodiments, the step of contacting comprises contacting the CRISRP-Cas complex and sample with a reporter susceptible to cleavage by the Cas protein collateral activity. In some embodiments, the step of contacting comprises incubating for a period of time above the temperature. In some embodiments, a detection method further comprises a step of amplifying nucleic acid present in the sample. In some embodiments, the step of amplifying utilizes a thermostable nucleic acid polymerase. In some embodiments, the steps of amplifying and contacting are performed in a single vessel.

In some embodiments, the Cas protein is a Cas12 protein. In some embodiments, the Cas protein has an amino acid sequence that is at least 80% identical to that of SEQ ID NO: 15. In some embodiments, the Cas protein has an amino acid sequence having at least 80%, sequence identity to any one of SEQ ID Nos. 3-21, 33-47, 51-56, 68-178, and 274-283. In some embodiments, the Cas protein has an amino acid sequence having 80%, sequence identity to any one of SEQ ID Nos. 1-283.

In some embodiments, in a method of performing a detection assay utilizing a Cas protein with collateral cleavage activity, the improvement that comprises utilizing a Cas protein with thermostable collateral cleavage activity. In some embodiments, the Cas protein is a Cas12 protein. In some embodiments, the Cas protein has an amino acid sequence that is at least 80% identical to that of SEQ ID NO: 15. In some embodiments, the Cas protein has an amino acid sequence having at least 80%, sequence identity to any one of SEQ ID Nos. 3-21, 33-47, 51-56, 68-178, and 274-283. In some embodiments, a method of performing a detection assay is conducted in a single reaction vessel. In some embodiments, the thermostable collateral cleavage activity is thermostable above a temperature of about 60° C. In some embodiments, the thermostable collateral cleavage activity is thermostable above a temperature of about 65° C. In some embodiments, the Cas protein has an amino acid sequence having at least 80% sequence identity to any one of SEQ ID Nos. 1-283.

### BRIEF DESCRIPTION OF THE DRAWING

FIGS. 1A and 1B document the insight, provided by the present disclosure that certain Cas13 protein(s) are insufficiently stable at relevant temperature(s), e.g., at temperatures at which nucleic acid extension and/or amplification reactions are typically performed (e.g., above about 60-65° C.).

FIG. 2 documents the insight, provided by the present disclosure that certain Cas12 protein(s) are insufficiently stable at relevant temperature(s), e.g., at temperatures at which nucleic acid extension and/or amplification reactions are typically performed (e.g., above about 60-65° C.).

FIGS. 3A-3C confirms and further demonstrate the thermostability of TccCas13collateralactivity.

FIG. 4 displays an exemplary method for discovery and screening of thermostable Cas enzyme candidates (e.g., Cas12 and Cas13 enzymes)

FIG. 5 displays an exemplary assessment of Cas12a candidate enzymes by endpoint assay.

FIG. 6 displays an exemplary assessment of Cas12a candidate enzymes by kinetic assay.

FIG. 7 displays an exemplary assessment of candidate enzymes at 58° C. by endpoint and kinetic assays.

FIG. 8 displays an exemplary assessment of candidate enzymes at 60° C. by endpoint and kinetic assays.

FIG. 9 displays an exemplary assessment of candidate enzymes at 62° C. by endpoint and kinetic assays.

FIG. 10 displays exemplary assessment of four candidate enzymes that were purified and activity was measured at varying temperatures (e.g., approximately 35° C. to approximately 65° C.).

FIG. 11 shows exemplary characterization of a subset of enzyme candidates using three different guide and target sets compared to no template control at both 58° C. and 70° C.

FIG. 12 shows exemplary characterization of a Cas12 candidate enzyme with multiple guide/target pairs at both 52° C. and 58° C.

FIG. 13 demonstrates kinetic assays for a Cas12a candidate enzyme, RS62, which shows activity at 52° C.

FIG. 14 displays characterization of Cas13 candidate enzymes by endpoint assay at both 37° C. and 52° C.

FIG. 15 displays exemplary thermostable Cas12a enzyme, RS9, requires Thermostable Inorganic Pyrophosphatase (TIPP) for amplification. Real time detection of ORF1ab amplification was completed over a range of starting concentrations of ORF1ab (4.5 copies/μL to 4,500 copies/μL in the presence or absence of TIPP.

FIG. 16 demonstrates exemplary thermostable Cas12a enzyme, RS9, is specific for its target and requires TIPP for amplification. Amplification was conducted with a starting concentration of 4,500 copies/p L ORF1ab template and either primers and guides specific to ORF1ab or non-targeting primers with an ORF1ab guide. Each reaction condition was also conducted in the presence or absence of TIPP.

FIG. 17 demonstrates exemplary thermostable Cas12a enzyme, RS9, displays collateral cleavage activity.

FIG. 18 demonstrates RS9 collateral cleavage activity compared to known Cas12a, LbaCas12a.

FIG. 19 demonstrates characterization of exemplary thermostable Cas13a enzyme, TccCas13a. Optimal temperature for TccCas13a activity was determined using a Cas reaction over a range of temperatures. Temperature profiles suggest TccCas13a shows highest activity at approximately 62° C.

FIG. 20 demonstrates TccCas13a can be activated by RNA, but cannot be activated by ssDNA, even at the highest concentrations of ssDNA.

FIG. 21 displays TccCas13a activation requires a higher concentration of ssDNA than RNA, similar to that observed for LwaCas13 ssDNA activation. TccCas13a was not activated by ssDNA* at any concentration (10 nM, 100 nM, or 1,000 nM) compared to control.

FIG. 22 demonstrates that TccCas13a shows increased collateral activity at “NN” sites compared to “UU” sites, while LwaCas13a shows no preference for collateral activity of “NN” sites compared to “UU” sites.

FIG. 23 demonstrates exemplary characterization of candidate thermostable Cas enzymes, Pal1, Pal2 low MW, Pal2 high MW, and Pal3.

FIG. 24 demonstrates exemplary Pal1 and Pal2 activity at 56° C.

FIG. 25 demonstrates exemplary activity of Pal1 at 37° C., 56° C., and 70° C. with different exemplary guides.

FIG. 26 demonstrates exemplary activity of Pal1 at 56° C. and 70° C. compared to control. These data suggest activity of Pal1 is specific to target DNA.

FIG. 27 demonstrates exemplary temperature profile of Pal1.

FIG. 28 demonstrates exemplary activity of Pal2 high MW at 37° C., 56° C., and 70° C. with different exemplary guides.

FIG. 29 demonstrates exemplary activity of Pal2 high MW at 56° C. compared to control. These data suggest activity of Pal2 high MW is specific to target DNA.

FIG. 30 demonstrates exemplary temperature profile of Pal2 high MW.

### DETAILED DESCRIPTION OF CERTAIN EMBODIMENTS

### Collateral Activity Assays

Those skilled in the art are well aware of the burgeoning plethora of useful detection (e.g., diagnostic) assays that have been and are being developed using Cas protein collateral activities. See, for example, Sashital Genome Med 2018:10, 32. Furthermore, those skilled in the art are well aware that a “detailed classification of CRISPR/Cas biosensing systems” based on Cas protein collateral activity has recently been made publicly available. See review by Li et al Trends Biotechnol. 37:730, July 2019.

Formats of particular interest include Cas13-based (e.g., Cas13a- or Cas13b-based) systems, including those referenced as “SHERLOCK” and/or “HUDSON” systems (see, for example, Gootenberg et al, Science 356:438, 2017; Gootenberg et al, Science 360:339, 2018; Myhrvold et al., Science 360:444, 2018; see also U.S. Ser. No. 10/266,887) and Cas12-based (e.g., Cas12a- or Cas12b-based) systems, including those references as “HOLMES” or “DETECTR” systems (see, for example, Cheng et al. CN patent filing CN107488710A; PCT/CN18/82769 and U.S. Ser. No. 16/631,157; Li et al. Cell Disc. 4:20, 2018; Chen et al. Science 360:436, 2018; Li, L. et al. bioRxiv Published online Jul. 26, 2018; U.S. Ser. No. 10/253,365). Both Cas13a and Cas13b enzymes have been used in SHERLOCK and/or HUDSON systems; similarly both Cas12a and Cas12b.

As is known in the art, and described in references cited herein, typical detection assays that utilize Cas protein collateral cleavage activity involve contacting an appropriate CRISPR-Cas complex, including a Cas protein with collateral activity and a guide RNA complementary to a target sequence of interest, with a sample that may contain the target sequence. Upon recognition of the target sequence, the Cas protein's collateral activity is activated, so that it cleaves unrelated nucleic acid (DNA or RNA or both, depending on the enzyme). A reporter of the relevant cleavable nucleic acid is provided, appropriately configured (e.g., labeled) so that its cleavage as a result of the activated collateral activity is detectable (e.g., separates a fluorophore from a quencher so that fluorescence becomes detectable, etc).

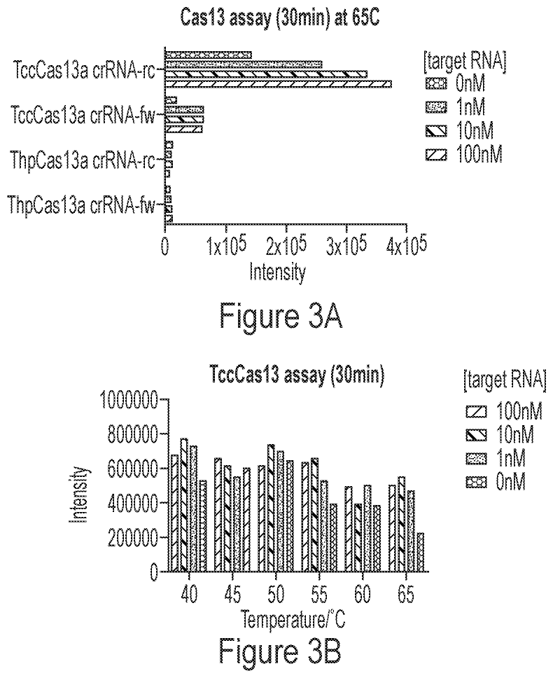

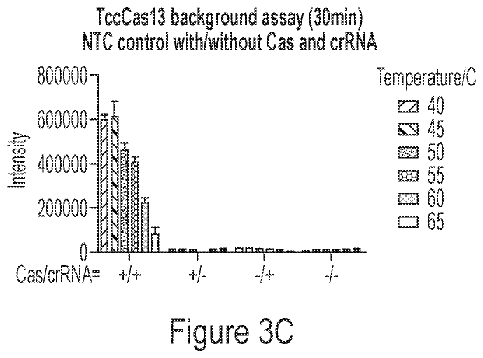

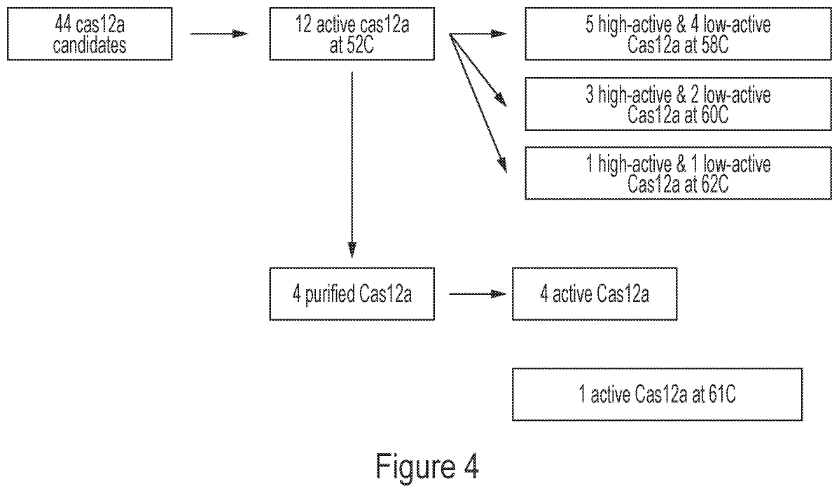

In many assays, a target sequence is generated and/or amplified (e.g., copied from RNA to DNA and/or amplified, for example by primer extension, DNA replication (e.g., by polymerase chain reaction) and/or transcription). See, for example, FIGS. 3 and 4 of the above-mentioned Li Review (Li et al Trends Biotechnol. 37:730, July 2019).

Thus, in many embodiments, a collateral activity assay includes steps of (1) target copying and/or amplification; (2) target binding; and (3) signal release and/or detection.

Typically, collateral activity assays as described herein are in vitro assays. In some embodiments, they may be cell free assays (e.g., may be substantially free of intact cells, or, in some embodiments, of cell fragments).

In some embodiments, collateral activity assays as described herein are performed on samples that are or are prepared from biological (e.g., blood, saliva, tears, urine, etc) or environmental (e.g., soil, water, etc) primary samples.

### Thermostable Cas Enzymes

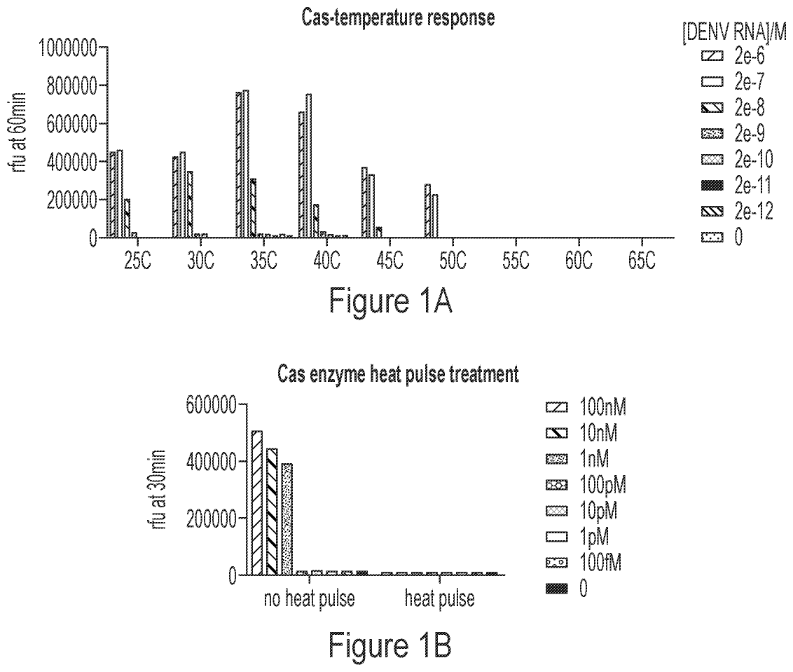

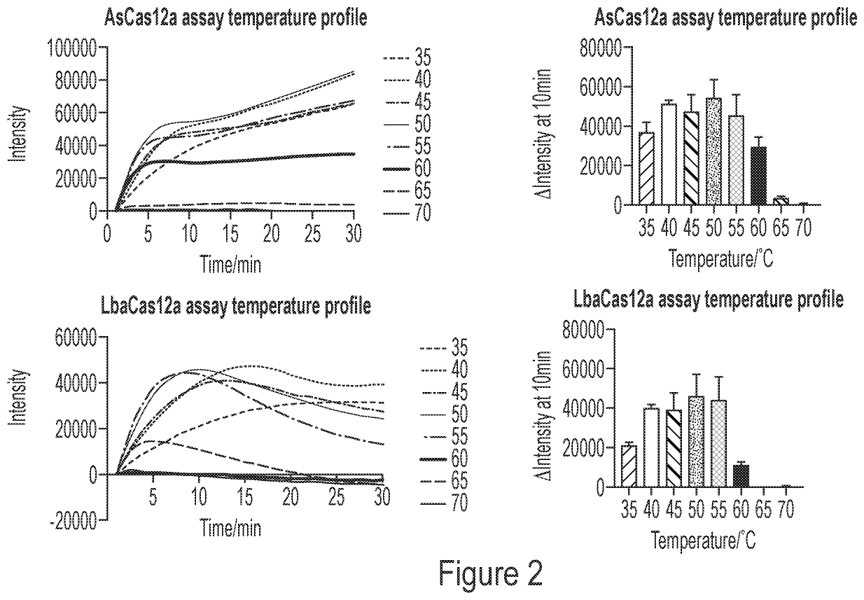

As described herein, the present disclosure identifies the source of a problem with certain detection (e.g., diagnostic assays) that utilize Cas protein collateral activity, as described above, in that certain Cas proteins with collateral activity are insufficiently stable at relevant temperatures (e.g., at temperatures at which nucleic acid extension and/or amplification are performed). Additionally, The present disclosure further surprisingly demonstrates that, for some proteins, loss of activity upon temperature elevation may be irreversible. This reality increases the significance of the insight, provided by the present disclosure, that Cas proteins with thermostable collateral activity are particularly desirable for use in assays asia described herein. FIGS. 1 and 2 document these findings.

The present disclosure therefore provides improved detection (e.g., diagnostic) assays that utilize Cas protein collateral activity, which improved assays utilize a thermostable Cas protein (e.g., whose collateral activity is thermostable) as described herein.

In some embodiments, steps of nucleic acid detection and target binding are performed in a single vessel; in some embodiments, steps of target binding an signal release are performed in a single vessel; in some embodiments, steps of steps of (1) target copying and/or amplification; (2) target binding; and (3) signal release and/or detection are performed in a single vessel; in some embodiments all steps are performed in a single vessel—i.e., provided improved assays are one-pot assays.

In some embodiments, improved collateral activity assays as described herein are in vitro assays. In some embodiments, they may be cell free assays (e.g., may be substantially free of intact cells, or, in some embodiments, of cell fragments).

In some embodiments, improved collateral activity assays as described herein are performed on samples that are or are prepared from biological (e.g., blood, saliva, tears, urine, etc) or environmental (e.g., soil, water, etc) primary sample.

In some embodiments, a Cas enzyme with thermostable collateral cleavage activity is a homolog (e.g., ortholog) of a Cas enzyme that either does not have demonstrable collateral cleavage activity, or has demonstrable collateral cleavage activity but loses such activity above a relevant temperature as described herein.

In some embodiments, a Cas enzyme with thermostable collateral cleavage activity as described herein is a Cas12 (e.g., Cas12a or Cas12b) enzyme. In some embodiments, a Cas enzyme with thermostable collateral cleavage activity as described herein is a Cas13 (e.g., Cas13a or Cas13b) enzyme.

In some embodiments, a Cas enzyme with thermostable collateral cleavage activity as described herein is a Cas enzyme comprising an amino acid sequence having 80%, 85%, 90%, 99% or 100% sequence identity to any one of SEQ ID Nos. 1-283. In some embodiments, improved collateral activity assays as described herein are performed using a Cas enzyme comprising an amino acid sequence having 80%, 85%, 90%, 99% or 100% sequence identity to any one of SEQ ID Nos. 1-283.

### Target Nucleic Acid

Those skilled in the art will immediately appreciate that technologies provided herein are broadly applicable to achieve detection of a wide range of nucleic acids including, for example, nucleic acids from an infectious agent (e.g., a virus, microbe, parasite, etc), nucleic acids indicative of a particular physiological state or condition (e.g., presence or state of a disease, disorder or condition such as, for example, cancer or an inflammatory or metabolic disease, disorder or condition, etc), prenatal nucleic acids, etc.

In some embodiments, a target nucleic acid is detected by an assay comprising a Cas enzyme as described herein and a cRNA. In some embodiments, the structure of the cRNA can affect the activity of the Cas/cRNA complex. In some embodiments the structure of the Cas/cRNA complex contributes to the thermostability of the Cas collateral activity.

Typically, provided technologies will be applied to one or more samples to assess presence and/or level of one or more target nucleic acids in the sample. In some embodiments, the sample is a biological sample; in some embodiments, a sample is an environmental sample. In some embodiments, a sample is a crude sample (e.g., a primary sample or a sample that has undergone minimal processing).

In some embodiments, a sample will be processed (e.g., nucleic acids will be partially or substantially isolated or purified out of a primary sample); in some embodiments, only minimal processing will have been performed (i.e., the sample will be a crude sample).

### Exemplification

### Example 1: Temperature Profile for LwaCas13a

The thermostability of LwaCas13a was tested. Briefly, labeled RNA target was incubated with Rnase Inhibitor; T7 RNA Polymerase, LwaCas13a, MgCl2 and a cRNA. Individual samples were incubated at various temperatures to determine collateral activity.

FIG. 1A presents a temperature profile for LwaCas13a collateral activity. As can be seen, low activity was observed above 45° C.; activity was completely abolished about 55° C.

Further, FIG. 1B presents results of testing the reversibility of loss of LwaCas13a at higher temperatures. LwaCas13a was incubated for 5 minutes at 65° C. (“heat pulse”) while the control group (“no heat pulse”) was incubated at room temperature. The active of both enzymes was then tested at 37° C. The heat pulse group shows no activity. This reality increases the significance of the insight, provided by the present disclosure, that Cas proteins with thermostable collateral activity are particularly desirable for use in assays as described herein.

### Example 2: Temperature Profile for AsCas12a and Lbacas12a

The thermostability of AsCas12a and Lbacas12a was tested. Briefly, labeled RNA target was incubated with Rnase Inhibitor; T7 RNA Polymerase, AsCas12a or Lbacas12a, MgCl2 and a cRNA. Individual samples were incubated at various temperatures to determine collateral activity.

FIG. 2 presents temperature profiles for AsCas12a and LbaCas12a. As can be seen, low AsCas12a activity is observed at temperatures greater than 55° C. AsCas12a remains active at 60° C. for ˜5 min. AsCas12a has <10% activity at 65° C. for a few minutes. Further, LbaCas12a activity is significantly diminished at temperatures greater than 55° C.

### Example 3: Exemplary Thermostable Cas Candidates

The present Example describes certain thermostable Cas13 candidates for use in improved collateral activity assays as described herein.

In the present Example, it was determined that a Cas13 with collateral activity thermostable within a temperature range of about 62-about 68° C. would be particularly desirable, among other things, in one-pot assays with LAMP pre-amplification.

We performed a computational search for potentially thermostable Cas candidates and identified:
2Cas13a candidates:
   TccCas13a (Thermoclostridium caenicola)

Exemplary sequences for use with TccCas13a include, but are not limited to:

(forward DR crRNAs)
(SEQ ID NO.: 284)
Agtgtctttgcaggaaagaacacagatcttgaggg
tcacaactcccatgtaggcggagactgcaacccct
atagtgagtcgtattaatt tc;
and
(reverse complement DR crRNAs)
(SEQ ID NO.: 285)
agtgtctttgcaggaaagaacacagatcttgaggg
ttgcagtctccgcctacatgggagttgtgacccct
atagtgagtcgtattaat ttc;

ThpCas13a (Thalassospira profundimaris)

Exemplary sequences for use with ThpCas13a include, but are not limited to:

(forward DR crRNAs)
(SEQ ID NO.: 286)
Tctttgcaggaaagaacacagatcttgaggggtgt
agttcccctcaatttggggatgaacgtcgacccct
atagtgagtcgtattaat ttc;
and
(reverse complement DR crRNAs)
(SEQ ID NO.: 287)
tctttgcaggaaagaacacagatcttgagggtcga
cgttcatccccaaattgaggggaactacaccccct
atagtgagtcgtattaa tttc;

4Cas12b candidates:
   AacCas12b (Alicyclobacillus acidoterrestris)
   AkCas12b (Alicyclobacillus kakegawensis)
   BhCas12b (Bacillus hisashii)
   LsCas12b (Laceyella sediminis)

Exemplary sequences for use with AacCas12b include, but are not limited to:

(crRNA)
(SEQ ID NO.: 288)
ttgtgagcggataaacacaggtgccacttctcaga
tttgagaagctcaacgggctttgccacctggaaag
tggccattggcacaccc gttgaaaaattctgtcc
tctagacccctatagtgagtcgtattaatttc

Exemplary sequences for use with AkCas12b include, but are not limited to:

(crRNA)
(SEQ ID NO.: 289)
Ttccggctcgtatgttgtgtggaattgtgagcgga
gtgccacttctcagaccgctcgccctatagtgagt
cgtattaatttc;
and
(tracrRNA)
(SEQ ID NO.: 290)
cgagcggtcatcttgaagccaacggggtgtttgct
cttggaaagagcacattggcacttcccgttgtcct
cgccgtcctatagacgac ccctatagtgagtcgt
attaatttc

Exemplary sequences for use with BhCas12b include, but are not limited to:

(crRNA)
(SEQ ID NO.: 291)
aattgtgagcggataaacacaggtgctaatgcctc
ccctatagtgagtcgtattaatttc;
and
(tracrRNA)
(SEQ ID NO.: 292)
gagacatcgtccagcaataggagtttctcacaccc
tgcagcacttatagctagacggttgtcctgaccaa
aagacagaacccctata gtgagtcgtattaattt
c

Exemplary sequences for use with LsCas12b include, but are not limited to:

(crRNA)
(SEQ ID NO.: 293)
Atggtcatagctgtttcctgtgtttatccgctcag
tgctaatcacatttaattcatctaccctatagtga
gtcgtattaatttc;
and
(tracrRNA)
(SEQ ID NO.: 294)
Gataaataatgtaatcctgtggttgaatggatttt
ttccatccttagcacacgcacagtattctttgccc
tttaggcaaaccctatagtg agtcgtattaattt
c.

Exemplary sequences of Cas proteins with thermostable collateral activity include those described in Table 1:

TABLE 1
Exemplary sequences of Cas proteins with thermostable collateral activity

| SEQ | | |
|---|---|---|
| ID | | |
| NO. | Cas Enzyme | Putative Sequence |
| 1 | TccCas13a | MKITKRKWGEHHPPLYFYRDEDSGRLLAQNDRKQDYTD |
| | (Thermoclostridium | TLFNDIAQDTFERSLRNRLLKTPEKGDKRFYSNEIVKLVE |
| | caenicola) | KLCQGADVAEIMKSMERNEKLRPKNEKEIKNLKKQLDG |
| | | TLSEYGKRYTAPEGAMTLNDALFYLVEGNPLKQAMAK |
| | | AELGKIREALIKEKENRINRVRYSIKNNKIPLRIQEDGGIT |
| | | PNNDRAAWLLGLMKPADPAKGITDCYPLLGELEEVFDF |
| | | DKLSKTLHEKISRCQGRPRSIAMAVDEALKQYLRELWEK |
| | | SPSRQQDLKYYFQAVQEYFKDNFPIRTKRMGARLRQELL |
| | | KDKTSLSRLLEPKHMANAVRRRLINQSTQMHILYGKLY |
| | | AYCCGEDGRLLVNSETLQRIQVHEAVKKQAMTAVLWSI |
| | | SRLRYFYQFEDGDILSNKNPIKDFRDKFLRDTNKYTHED |
| | | VEACKEKLQDFFPLKELQEKIKEDAKGLQETDNKQADTT |
| | | DFKAIGHIVRDDRKLCNQLLAECVSCIGELRHHIFHYKN |
| | | VTLIQALKRIADKVKPEDLSVLRAIYLLDRRNLKKAFAK |
| | | RISSMNLPLYYREDLLSRIFKKEGTAFFLYSAKIQMTPSFQ |
| | | RVYERGKNLRREFECERMKAEASNGQNGQDGDRLKWF |
| | | RQLAAGDSADTHFNWAVEAYAESAADVENNVEFDTDV |
| | | DAQRALRNLLLLIYRHHFLPEVQKDETLVTGKIHKVLER |
| | | NRQLSEGQGPNQGKAHGYSVIEELYHEGMPLSDLMKQL |
| | | QRRISETERESRELAQEKTDYAQRFILDIFAEAFNDFLEA |
| | | HYGEEYLEIMSPRKDAEAAKKWVKESKTVDLKTSIDEK |
| | | EPEGHLLVLYPVLRLLDERELGELQQQMIRYRTSLASWQ |
| | | GESNFSEEIRIAGQIEELTELVKLTEPEPQFAEEVWGKRA |
| | | KEAFEDFIEGNMKNYEAFYLQSDNNTPVYRRNMSRLLR |
| | | SGLMGVYQKVLASHKQALKRDYLLWSEKHWNVKDEN |
| | | GADISSAEQAQCLLQRLHRKYAESPSRFTEEDCKLYEKV |
| | | LRRLEDYNQAVKNLSFSSLYEICVLNLEILSRWVGFVQD |
| | | WERDMYFLLLAWVRQGKLDGIKEEDVRDIFSEGNIIRNL |
| | | VDTLKGENMNAFESVYFPENKGSKYLGVRNDVAHLDL |
| | | MRKNGWRLEAGKTCSVMEDYINRLRFLLSYDQKRMNA |
| | | VTKTLQQIFDRHKVKIRFTVEKGGMLKIEDVTADKIVHL |
| | | KGSRLSGIEIPSHGERFIDTLKALMVYPRG* |
| 2 | ThpCas13a | MRIIKPYGRSHVEGVATEQPRRKLRLNTRPDISRDIPGFA |
| | (Thalassospira | QSHDALIIAQWISAIDKIATKPKPDQKPTQRQMNLRTTLG |
| | profundimaris) | DAAWQHLMAKNLLPAAKDPAIREKLHLIWQSKIAPWGA |
| | | SRPQEEKRGKPTPKGGWYERFCGALSPEAITQNVARQIA |
| | | KDIYDHLYVAAKRKGREPVKQGESSNKPGKFKPDRKLS |
| | | LIEERAESIAKNALRPGTHAPCPWGQDDQAIYEQAGDVA |
| | | TKIYDDARDYLEDKKRRSGNRNTSSVQYLPRDLAVKILY |
| | | AQYGRVFGPDTTIKAALDEQQSLFALHTAIKDCYHRLVN |
| | | DARKRHILRILPRNMAALFRLVRAQYDNRDINALIRLGK |
| | | VIHYHAGEQGKDEHHGIRDYWPSQQDIQNSRFWGSDGQ |
| | | ADIKRHEAFSRIWRHIIALASRTLHDWADPDSQKFTGDD |
| | | DDILMRAGAIESNVWDAGRYERKCDVLFGAQASLFCGA |
| | | EDFEKATLKQAITGTGNLRNATFHFKGKARFENELQRLA |
| | | DDVPVDVQSAIAALWQKDAEGRTRQIAETLQAVLAGHF |
| | | LSERQNRHILATLMAAMAQPGDVPLPRLRRVLARHDSIC |
| | | QRGRILPLPPCPDRAKLEESPALTCQYTVLKMLYDGPFR |
| | | AWLAQQNSTILNHYIDSTIARTNKAAQDMLAPAE |
| | | KDLITARAADIPRLSVDEKMVDFLGRLTAATATEMRVQ |
| | | RGYQSDGEKAQKQAGYIGEFECDVIARAFSDFLGQSGFD |
| | | FVLKLKADTPKPDAAQCDVAALIAPGDVPALTPQAWQQ |
| | | VLYFILHLVPVDDASRLLHQTRKWQALEKKGKDKEVKK |
| | | EKDKEVKKEDEKPDIADLQSVLMLYLDMHDAKFTGGA |
| | | ALHGIEKFAEFFVEKADFRAVFPPQSLQDQDRSIPRRGLR |
| | | EIVRFGHLPLLQHMSGTVKITHDNVVAWQTARTPDATG |
| | | TSPIARRQKQREELHALAVERPARFRNADLHNYMHALV |
| | | DVIKHRQLSAQVTLSDQVRLHRLMMGVLGRLVDYAGL |
| | | WERDLYFVLLALLYHHGVTPDDVLKGQGKRKLADGQV |
| | | VEALKPKNRKAAAPVGVFDDLDHYGIYQDDRQSIRNGL |
| | | SHFNMLRGGTAPDLSHWVNQTRRLVAHDRKLKNAVAK |
| | | SVIEMLAREGFDLDWTIEPDSGKHILRHGKIRTRQAQHFQ |
| | | KSRIRIEKKSAKPDKNDTVKIRENLHGDAMVERVARLFA |
| | | ARAQKYRDITTEKRLDHLFLKPKG* |
| 3 | AacCas12b | MAVKSIKVKLRLDDMPEIRAGLWKLHKEVNAGVRYYT |
| | (Alicyclobacillus | EWLSLLRQENLYRRSPNGDGEQECDKTAEECKAELLERL |
| | acidoterrestris) | RARQVENGHRGPAGSDDELLQLARQLYELLVPQAIGAK |
| | | GDAQQIARKFLSPLADKDAVGGLGIAKAGNKPRWVRM |
| | | REAGEPGWEEEKEKAETRKSADRTADVLRALADFGLKP |
| | | LMRVYTDSEMSSVEWKPLRKGQAVRTWDRDMFQQAIE |
| | | RMMSWESWNQRVGQEYAKLVEQKNRFEQKNFVGQEH |
| | | LVHLVNQLQQDMKEASPGLESKEQTAHYVTGRALRGSD |
| | | KVFEKWGKLAPDAPFDLYDAEIKNVQRRNTRRFGSHDL |
| | | FAKLAEPEYQALWREDASFLTRYAVYNSILRKLNHAKM |
| | | FATFTLPDATAHPIWTRFDKLGGNLHQYTFLFNEFGERR |
| | | HAIRFHKLLKVENGVAREVDDVTVPISMSEQLDNLLPRD |
| | | PNEPIALYFRDYGAEQHFTGEFGGAKIQCRRDQLAHMHR |
| | | RRGARDVYLNVSVRVQSQSEARGERRPPYAAVFRLVGD |
| | | NHRAFVHFDKLSDYLAEHPDDGKLGSEGLLSGLRVMSV |
| | | DLGLRTSASISVFRVARKDELKPNSKGRVPFFFPIKGNDN |
| | | LVAVHERSQLLKLPGETESKDLRAIREERQRTLRQLRTQ |
| | | LAYLRLLVRCGSEDVGRRERSWAKLIEQPVDAANHMTP |
| | | DWREAFENELQKLKSLHGICSDKEWMDAVYESVRRVW |
| | | RHMGKQVRDWRKDVRSGERPKIRGYAKDVVGGNSIEQI |
| | | EYLERQYKFLKSWSFFGKVSGQVLRAEKGSRFAITLREHI |
| | | DHAKEDRLKKLADRIIMEALGYVYALDERGKGKWVAK |
| | | YPPCQLILLEELSEYQFNNDRPPSENNQLMQWSHRGVFQ |
| | | ELINQAQVHDLLVGTMYAAFSSRFDARTGAPGIRCRRVP |
| | | ARCTQEHNPEPFPWWLNKFVVEHTLDACPLRADDLIPTG |
| | | EGEIFVSPFSAEEGDFHQIHADLNAAQNLQQRLWSDFDIS |
| | | QIRLRCDWGEVDGELVLIPRLTGKRTADSYSNKVFYTNT |
| | | GVTYYERERGKKRRKVFAQEKLSEEEAELLVEADEARE |
| | | KSVVLMRDPSGIINRGNWTRQKEFWSMVNQRIEGYLVK |
| | | QIRSRVPLQDSACENTGDI |
| 4 | AkCas12b | MAVKSIKVKLRLSECPDILAGMWQLHRATNAGVRYYTE |
| | (Alicyclobacillus | WVSLMRQEILYSRGPDGGQQCYMTAEDCQRELLRRLRN |
| | kakegawensis) | RQLHNGRQDQPGTDADLLAISRRLYELLVLQSIGKRGDA |
| | | QQIASSFLSPLVDPNSKGGRGEAKSGRKPAWQKMRDQG |
| | | DPRWVAAREKYEQRKAVDPSKEILNSLDALGLRPLFAVF |
| | | TETYRSGVDWKPLGKSQGVRTWDRDMFQQALERLMSW |
| | | ESWNRRVGEEYARLFQQKMKFEQEHFAEQSHLVKLARA |
| | | LEADMRAASQGFEAKRGTAHQITRRALRGADRVFEIWK |
| | | SIPEEALFSQYDEVIRQVQAEKRRDFGSHDLFAKLAEPKY |
| | | QPLWRADETFLTRYALYNGVLRDLEKARQFATFTLPDA |
| | | CVNPIWTRFESSQGSNLHKYEFLFDHLGPGRHAVRFQRL |
| | | LVVESEGAKERDSVVVPVAPSGQLDKLVLREEEKSSVAL |
| | | HLHDTARPDGFMAEWAGAKLQYERSTLARKARRDKQG |
| | | MRSWRRQPSMLMSAAQMLEDAKQAGDVYLNISVRVKS |
| | | PSEVRGQRRPPYAALFRIDDKQRRVTVNYNKLSAYLEEH |
| | | PDKQIPGAPGLLSGLRVMSVDLGLRTSASISVFRVAKKEE |
| | | VEALGDGRPPHYYPIHGTDDLVAVHERSHLIQMPGETET |
| | | KQLRKLREERQAVLRPLFAQLALLRLLVRCGAADERIRT |
| | | RSWQRLTKQGREFTKRLTPSWREALELELTRLEAYCGR |
| | | VPDDEWSRIVDRTVIALWRRMGKQVRDWRKQVKSGAK |
| | | VKVKGYQLDVVGGNSLAQIDYLEQQYKFLRRWSFFARA |
| | | SGLVVRADRESHFAVALRQHIENAKRDRLKKLADRILM |
| | | EALGYVYEASGPREGQWTAQHPPCQLIILEELSAYRFSD |
| | | DRPPSENSKLMAWGHRGILEELVNQAQVHDVLVGTVYA |
| | | AFSSRFDARTGAPGVRCRRVPARFVGATVDDSLPLWLTE |
| | | FLDKHRLDKNLLRPDDVIPTGEGEFLVSPCGEEAARVRQ |
| | | VHADINAAQNLQRRLWQNFDITELRLRCDVKMGGEGTV |
| | | LVPRVNNARAKQLFGKKVLVSQDGVTFFERSQTGGKPH |
| | | SEKQTDLTDKELELIAEADEARAKSVVLFRDPSGHIGKG |
| | | HWIRQREFWSLVKQRIESHTAERIRVRGVGSSLD |
| 5 | BhCas12b | MATRSFILKIEPNEEVKKGLWKTHEVLNHGIAYYMNILK |
| | (Bacillus | LIRQEAIYEHHEQDPKNPKKVSKAEIQAELWDFVLKMQK |
| | hisashii) | CNSFTHEVDKDEVFNILRELYEELVPSSVEKKGEANQLS |
| | | NKFLYPLVDPNSQSGKGTASSGRKPRWYNLKIAGDPSW |
| | | EEEKKKWEEDKKKDPLAKILGKLAEYGLIPLFIPYTDSNE |
| | | PIVKEIKWMEKSRNQSVRRLDKDMFIQALERFLSWESW |
| | | NLKVKEEYEKVEKEYKTLEERIKEDIQALKALEQYEKER |
| | | QEQLLRDTLNTNEYRLSKRGLRGWREIIQKWLKMDENE |
| | | PSEKYLEVFKDYQRKHPREAGDYSVYEFLSKKENHFIWR |
| | | NHPEYPYLYATFCEIDKKKKDAKQQATFTLADPINHPLW |
| | | VRFEERSGSNLNKYRILTEQLHTEKLKKKLTVQLDRLIYP |
| | | TESGGWEEKGKVDIVLLPSRQFYNQLFLDIEEKGKHAFTY |
| | | KDESnCFPLKGTLGGARVQFDRDHLRRYPHKVESGNVG |
| | | RIYFNMTVNIEPTESPVSKSLKIHRDDFPKVVNFKPKELT |
| | | EWIKDSKGKKLKSGIESLEIGLRVMSIDLGQRQAAAASIF |
| | | EVVDQKPDIEGKLFFPIKGTELYAVHRASFNIKLPGETLV |
| | | KSREVLRKAREDNLKLMNQKLNFLRNVLHFQQFEDITER |
| | | EKRVTKWISRQENSDVPLVYQDELIQIRELMYKPYKDW |
| | | VAFLKQLHKRLEVEIGKEVKHWRKSLSDGRKGLYGISLK |
| | | NIDEIDRTRKFLLRWSLRPTEPGEVRRLEPGQRFAIDQLN |
| | | HLNALKEDRLKKMANTIIMHALGYCYDVRKKKWQAKN |
| | | PACQIILFEDLSNYNPYEERSRFENSKLMKWSRREIPRQV |
| | | ALQGEIYGLQVGEVGAQFSSRFHAKTGSPGIRCSWTKE |
| | | KLQDNRFFKNLQREGRLTLDKIAVLKEGDLYPDKGGEK |
| | | FISLSKDRKCVTTHADINAAQNLQKRFWTRTHGFYKVY |
| | | CKAYQVDGQTVYIPESKDQKQKIIEEFGEGYFILKDGVY |
| | | EWVNAGKLKIKKGSSKQSSSELVDSDILKDSFDLASELK |
| | | GEKLMLYRDPSGNVFPSDKWMAAGVFFGKLERILISKLT |
| | | NQYSISTIEDDSSKQSM |
| 6 | LsCas12b | MSIRSFKLKIKTKSGVNAEELRRGLWRTHQLINDGIAYY |
| | (Laceyella | MNWLVLLRQEDLFIRNEETNEIEKRSKEEIQGELLERVHK |
| | sediminis) | QQQRNQWSGEVDDQTLLQTLRHLYEEIVPSVIGKSGNAS |
| | | LKARFFLGPLVDPNNKTTKDVSKSGPTPKWKKMKDAGD |
| | | PNWVQEYEKYMAERQTLVRLEEMGLIPLFPMYTDEVGD |
| | | IHWLPQASGYTRTWDRDMFQQAIERLLSWESWNRRVRE |
| | | RRAQFEKKTHDFASRFSESDVQWMNKLREYEAQQEKSL |
| | | EENAFAPNEPYALTKKALRGWERVYHSWMRLDSAASEE |
| | | AYWQEVATCQTAMRGEFGDPAIYQFLAQKENHDIWRG |
| | | YPERVIDFAELNHLQRELRRAKEDATFTLPDSVDHPLWV |
| | | RYEAPGGTNIHGYDLVQDTKRNLTLILDKFILPDENGSW |
| | | HEVKKVPFSLAKSKQFHRQVWLQEEQKQKKREVVFYD |
| | | YSTNLPHLGTLAGAKLQWDRNFLNKRTQQQIEETGEIGK |
| | | VFFNISVDVRPAVEVKNGRLQNGLGKALTVLTHPDGTKI |
| | | VTGWKAEQLEKWVGESGRVSSLGLDSLSEGLRVMSIDL |
| | | GQRTSATVSVFEITKEAPDNPYKFFYQLEGTELFAVHQRS |
| | | FLLALPGENPPQKIKQMREIRWKERNRIKQQVDQLSAILR |
| | | LHKKVNEDERIQAIDKLLQKVASWQLNEEIATAWNQAL |
| | | SQLYSKAKENDLQWNQAIKNAHHQLEPVVGKQISLWRK |
| | | DLSTGRQGIAGLSLWSIEELEATKKLLTRWSKRSREPGV |
| | | VKRIERFETFAKQIQHHINQVKENRLKQLANLIVMTALG |
| | | YKYDQEQKKWIEVYPACQVVLFENLRSYRFSYERSRRE |
| | | NKKLMEWSHRSIPKLVQMQGELFGLQVADVYAAYSSR |
| | | YHGRTGAPGIRCHALTEADLRNETNIIHELIEAGFIKEEHR |
| | | PYLQQGDLVPWSGGELFATLQKPYDNPRILTLHADINAA |
| | | QNIQKRFWHPSMWFRVNCESVMEGEIVTYVPKNKTVHK |
| | | KQGKTFRFVKVEGSDVYEWAKWSKNRNKNTFSSITERK |
| | | PPSSMILFRDPSGTFFKEQEWVEQKTFWGKVQSMIQAYM |
| | | KKTIVQRMEE |
| 7 | Cas12 | MFKKKLFDDEEFISLAQNQEESNALNAFKGFTTHFKDFQENRKNMYS |
| | | EDKESTAIAYRIIHENLPVFITNNIRFEKIINELDRSNIHSIEKELKEELANN |
| | | KLKDIFNIEYFQNTLTQNDITRYNTIIGGKVKADGKKVQGLNEYINLFN |
| | | QHNKDKKLPLLKPLYKQILSEENSASFIVPAFEKDNEVLQSIFDFWNKCI |
| | | IDAKGPISGKKYNLLSKIQSLLQNLDKLKNNQLEEMYFENENLSTISNDV |
| | | YGQWNLIRDALGNFYNSIDAKKNKKDYYSWKEIQDALVYYKQTNDEY |
| | | KDIDQKAFLIYFKEMKVNDGEENTNNNIINLINERYKRIEPLLKEDRDN |
| | | RKDLHQDKGKVAIIKEFLDSLKLLQNTIKLLYVDDSLDNMNYDFYNQLT |
| | | DYYETLRPLNTLYNRVRNYMTRKPFSEEKFVLTFNSPTLLDGWDLNKE |
| | | EANLGVILRKDNKYYLGIMNKGDNKIFKKYDEEPGDDYYEKMVYKLLP |
| | | GPNRMLRKVFFSNKNIEYYKPNQDIQNLYNKGEFKKGESLNKESLHKLI |
| | | DFYKNSISKNGDWSVFNFKFKKTTAYDDISQFYKDVENQGYKLFFKTIK |
| | | TSYIDQLVNEGKLYLFQIYNKDFSENKKRKDESNPNLHTIYFKNLFSEDN |
| | | LKNVVYKLNGKAEVFYRKKSIEYPEEIRRKGHHYNELKDKFDYPIIKDKR |
| | | YSEDKFLFHVPITLNFLAKSDEKVNEMVKNYIAATNEKIHIIGIDRGERN |
| | | LLYLSLIDSNGNIVKQQSLNIIELPKYQKQIDYHAKLNEKEKQRLAARQN |
| | | WDVIENIKELKEGYLSQVIHQIARLMVDYKAILVMEDLNFGFKRGRFK |
| | | VEKQVYQKFEKMLIDKLSYLVFKEKNLCEPGGSLRAYQLSAPFKSFKAL |
| | | GKQSGMIFYVPAQYTSKIDPTTGFYNFLNIDVSNLARSKETFSKFDKIVY |
| | | NKKEDYFEFYCKMINFESANQLTKKSQNKANAELKEFQWILCSTHHDR |
| | | FKVERKNNQINYCKINVNEELKKLLNSKGINYEKSNDLKSEILNIDESKFF |
| | | KELGYLLKILVSLRYNNGKKGSEEQDFILSPVKNASGKFFCTLDNNNTLP |
| | | LDADANGAYNIALKGLMIVQRVKAGGKLDLSISKDDWINFLIMNKKLP |
| | | K |
| 8 | Cas12 | MSNQSVFKDFTNLYELSKTLRFELKPVGKTLRMLEDAKVFKTDELIQKK |
| | | YEQTKPFINKLHQEFVKESLEGRSLEGLESYQDILKEWQKDKKDKIAQK |
| | | NLGIKEKELYKQVTQLFNAKAKEWSEPYAHLGLKKKDIGILFEEGVFKIL |
| | | KEKYNNDKDAKITNKVTGEIFFEDFWKGFVGYFQKFFETRKNFYKDDG |
| | | TSTAIATRIVAQNLKRFCDNIGLFEKIKDQIDSSEVEQSFGISMEKVFSLD |
| | | FYNQCLLQGGIDKYNEILGGKTLENGEKFKGINELINKYRQDNKGDKSS |
| | | FLKILDKQILSEKESFIDEIKNDKELEETLKNLHETAKVKTKIFGTLFEDFIG |
| | | NNTKYDLAKIYISKEAFNTISHKWTGGTDLFAENLFNALKDEQILKSSAK |
| | | KKDGSYVFPDFIEFLHIKTALENVPKDINFWKERYYVNKEGENKEFFLG |
| | | NGEIWQQFLQIFNFEFNELFQKEIIDNQTGKKMHIGYKVYKEEISKLLE |
| | | DFKVDKDSTVIIKHFADSVLWIYQMAKYFALEKKRTWRDEYDLDTFYT |
| | | DPKNGYLAFYENAYEEIVQIYNKLRNYLTKKPYSTEKWKLNFQNSTLAS |
| | | GWDKNKEADNFTVILRKDGKYFLGLMRKGANKLFDKRYGSEFSQGLE |
| | | KGKYEKMNYKYFPSPSKMIPKTSTQVHEVKKHFKNSSEPFFLEESSSLG |
| | | KFIKQLKITKEVFDLNNFEYKKSYLSTLNGESPDESQRVKADSKKTGQV |
| | | KLFQKEFLNLSQNELLYKKSLFAWVDFCKEYLDCFPSTGDGFLQFKKYI |
| | | QDTEKYESIDQFYKDIERGGYKISFQNISEEYISCKNQNSELYLFKIHNKD |
| | | WNLKDGKPKTGMKNLHTMYFESLFSSENIAQNFPMKLNGQAEIFYRP |
| | | KTDINKLEMKKDSKGKNVVDHKRYEEDKIFFHLPMTLNRGKSLFNFNV |
| | | QLNNFLADNPEINIIGVDRGEKHLAYYSVINQNQEILDGGTLNVVKGG |
| | | NGKDIDYHKKLEDKAEKREQARKDWQDVEGIKDLKKGYISQVVRKLA |
| | | DLAIEHNAIIVFEDLNMRFKQIRGGIEKSVYQQLEKALIEKLSFLVRKNE |
| | | KNPEEAGYLLKAYQLSAPFETFQRIGKQTGIIFYTQASYTSKIDPLTGWR |
| | | PNLYLKYSNAKKAKADISKFSEIEFINNRFEFTYDLQEFRSQKDKKKEYP |
| | | KKTLWTLCSSVERYRWNRKLNDNKGGYEHYSDLTSDFKKLFKKYNINI |
| | | NEDILGQIENMDTDDRKNNARFFSGFMFFWNLICQIRNTNSDVISGE |
| | | SDNDFILSPVEPFFDSRKASQFGSDLPENGDDNGAFNIARKGIMILKKI |
| | | SQYVEENENCDKLKWGDLYISHTDWDNFI |
| 9 | Cas12 | MTNYTDFIGLYPVQKTLRFELRPQGKTAEKMRESGLLEQDREKAKNYI |
| | | VMKALIDDYHRRFINELLEKASFDWQPLFEALNNVKVNKDDKSKKELE |
| | | KEQLHMRKELIGLFEKDERFKYLFSEKLFSELLNKEISERNDPDEMEAM |
| | | RSFDRFSGYFIGFHENRRNIYSNEDKHNSLAYRVVAENFPKFADNCRK |
| | | YSLIKENMQEAVVEFKKEIASVVDIDVDQMFDISYFNKVLTQKGIDDY |
| | | NTMLGGVSEEGSVKIRGLNEFLNLYYQKVTDNKRIKMAPLYKQILCESK |
| | | TKSFIPYMFENDEEVISSINQYYDSVKYDILQRSVYLLSNYKEYDASKIFI |
| | | DQKSISSISIVLFGSWETLGGLMQIYKADQIGDPGLEKTRKKVDKWLSS |
| | | SYFTLKEVFEAIGEQDPFRVYVEKLSLVLKNIEEFDKSCLLEGTHFSGDEL |
| | | LTQDIKGFLDLLMEVQHLMKPFNAKEDLDKDAAFYSEYNEIYEALSEIIP |
| | | LYNKVRNYATKKKYSTYKIKMNFGNPTLAAGWDLNKERDNTAVILLR |
| | | GNNYYLGIMNPKKKTKFEELPSGEDNDCYRKMVYKLLPGPNKMLPKV |
| | | FFSKKGIGTFNPSKEILEGYETGKHKLGDSFDIDYCHSLIDFFKENIPKYG |
| | | DWGTYEFKFSPTEEYSDISQFYKEVSEQGYKITFQNISRKAIDDLVNNG |
| | | ALFLYQIYNKDFSEHSKGKNNLHTMYWKAAFSEENLRNVVIKINGEAE |
| | | LFYRDKSDISKTEHSAGTILVNRTDRKDNPIPNSIYYELFKYKTGQIKSVS |
| | | DEAKQYLDDLVTHEAKYPITKDRRYTEDRMFFHIPITLNFGSSGNTNIN |
| | | KAVIDHVLNSKDVHIIGIDRGERNLLYVSVIDRKGNIIKQRSLNVIDGIDY |
| | | HEKLDQREKENISARKSWSNVEKIKDLKEGYLSYVIH |
| 10 | Cas12 | MKDFYQFTNLYALSKTLRFSLIPTPATKQMLEDAKVFEKDETIQKKYEA |
| | | TKPYFDRLHREFALEALQDQKLDFKNYLELYRKYKADKKASGKLLINIEK |
| | | DLRKEVVKLFDKQGEKWAKQYPGLKNKNIGVLFKEAVFTVILKERYGN |
| | | EKETQILDESSGQLVSIFDSWKGFIGYFKKFHETRKNFYKDDGTSTALA |
| | | TRIIDQNLKRFCDNILIFESTKEKVDFSEVEISFGKPLSEVFTLEFYNTCFL |
| | | QNGIDFYTKILGGETLQNGEKVKGLNECINLHKQKTGEKLPFFKSLDKQ |
| | | ILSEKDKFFIDEISNETQLLEVLKSFVASAESKTDTIKTLVDDFVKDQDKY |
| | | DLNYIYFSNDGLNTITRKWTTETQVFEEALYTALKAAKVVSSSAKKNEG |
| | | GYSFPDFIPFAHLKTALESIKIDGTIWRDNFNAIENFEEKSIWAQFLAIY |
| | | NFELSNLFETEIKNPEIGNCPTIGYNVYKQDFEELLKSFVYDPNAKVTIK |
| | | NFADNVLSIYQMAKYFAVEKKRGWNTDYELDVFYTDPQNGYLQYYE |
| | | NAYEEIVQVYNKLRNYLTKKPYSEEKWKLNFDSGTPIKYTTRAIIFNNTT |
| | | NERYYLGLLKKGVAKPREFEPINNNIISSGEFRRMIIQQLKFQTLAGKGY |
| | | VRDFGVKYSEDKDGVKHLQQLIKKQYLSKYPCLKKIADGVYNDKKAFD |
| | | ADIKDVLLETYNLDFQPISEEFILNKNRLGEIYLFEIHNKDWNLKDGKNK |
| | | SGSKNLHTMYFESLFVDKTTFKLNNEGAEVFYRPATNEGKLGTKKDRN |
| | | GKIIINHKRYATDKILFHCPIGLNKDAGKSYTFNAKINNMLANNPDINII |
| | | GVDRGEKHLAYYSVITQKGKILDRGSLNKVEGGDKQEIDYAKKLEETAK |
| | | NREQARKDWQAVEGIKDLKRGYISQVVRKLADLAIEHNAIIVFEDLNM |
| | | RFKQIRGGIEKSVYQQLEKALIDKLSFLVMKGEADPEKAGHLLKAYQLV |
| | | APFESFQSMGKQTGIIFYTQANYTSKIDPITGWRPNLYLKYTSAEKAKA |
| | | DILKFSKIEFVNNRFELTYDIKNFVLDKKVVLSNKTKWTVCSSVERFRW |
| | | NRRLESNQGNYEHYENLTENLSSLFKDFGFEIEQNIIRQVEQLATKGNE |
| | | QFFRSFIFYVNLIFQIRNTDAKAKDQNKEDFILSPVEPFFDSRTPEKFGE |
| | | NLPENGDDNGAFNIARKGIIMLNKISAYKQEVGNVDKIIWKDLFISAAE |
| | | WDNFTQE |
| 11 | Cas12 | MDSYEQFTKLYPIQKTIRFELKPQGRTKEHFDNSNFLEKDRERDDNYKI |
| | | LKEVIDDYHREFIDECLSNIQLNWDDLKKFSEEYRRSKEKKNNRDSESE |
| | | QKRMSTTSETRAINKKNLEAEQKRMRGEIVSAFKKDDRFKHLFSEKLFS |
| | | ILLKNQIYEKGTLEEIEAFDCFNKFSGYFKSFHENRKNMYSDEDKETAIS |
| | | YRIINENFPKLLDNFEKYQYVCREYPEQIREAESTLAEAGCYIKMDEIFSI |
| | | DNFNNVMMQGGKESGISRYNLAIGGIVQGTGEKPKGLNEFLNLAYQ |
| | | NEPNGRKKIRMEPLYKQILSKEESFSYRLEAFTDDSQLLSAIRSFFDIVEK |
| | | DKNGNIFDRAVNLMSSFSNYDTSKIYIRKAYLNQVSKEIFGYRGKSDSK |
| | | PAKTADESLNKSGGWEKLGQMLRDYKADSIGDRNLEKTCKKVDKWL |
| | | DSDEFTLSDILGAISLAGSNETFEAYVSEICVARRNIDKEKEKEKNINVEK |
| | | ISGDTESIQIIKALLDSVQEFFHLLSPFQLHPNTPHDWTFYAEFNDIYDK |
| | | LSAITPLYNQARNHLTKKNLDTSKIKLNFNNPTLANGWDVNKEYENTA |
| | | VILIRDGKYYLGIMNPKNKRKIKFDEGSGAGPFYQKMVYKLLPGPYRM |
| | | LPKVFFAKKNIDYYNPSQEIREGYKAGKHKKGKEFDKGFCHKLIDFFKES |
| | | IQKNENWKVFDFKFSPTESYDDISEFYQEVEKQGYRMYFVNIPSDTIDR |
| | | YVEGGDMFLFQIYNKDFAKGAKGNKDMHTLYWNAVFSEENLQKGV |
| | | MKLSGEAELFYRKKSDIKDPPHREGEILVNRTYIDRTHVSGVMGEQNT |
| | | VKESRIPVPDEIHKNLFDYYNHGRELTKEEKEYCDKVGSFKAYYGIVKD |
| | | RRYLENKMYFHVPLTLNFKAIGEKRINKMAIEKFLTDENACIIGIDRGER |
| | | NLLYYSIIDRNGKIIDQKSLNVIDGFDYHEKLSQRQTEREVARQSWNSI |
| | | GKIKDLKEGYLAKAVHEISKMAIKYNAIVVLEDLHFGFKKGRLKVEKQIY |
| | | QKFEEMLINKLNYLVFKDVSDSSDAGGVLNAYQLTAPLESFSKLGKQS |
| | | GILFYVPAAFTSVIDPTTGFVDLFNSSSITSTQKKKEFLQRFESIVYSARD |
| | | GGIFAFTFDYRNFSKIATDHRNMWTVYTHGERIRYVRDEKCYKTTDPT |
| | | KRIKEALSGIEYDDGSDIRDKITQSGDNNLINTVYHSFMDTIKMRNKD |
| | | GRIDYIISPVKNRNGEFFRSDYKHRDFPVDADANGAYHIALKGELLMR |
| | | MIGKTYDSNSDKMPKLEHKDWFEFMQTRGDQ |
| 12 | Cas12 | MKKEKEFKSFGDFTNLYEISKTLRFELKPVENTQTMLDEADVFGKDKVI |
| | | KDKYTKTKPFIDKLHREFVDESLKDVSLSGLKKYSEVLENWKKNKKDKD |
| | | IVKELKKEEERLRKEVVEFFDNTAKKWANEKYKELGLKKKDIGILFEESV |
| | | FDLLKEKYGEEQDSFLKEEKGDFLKNEKGEKVSIFDEWKGFVGYFTKFQ |
| | | ETRKNFYKNDGTETALATRIIDQNLKRFCDNIDDFKKIKNKIDFSEVEKN |
| | | FNKTADVFSLDFYNQCLLQKGIDSYNEFIGGKTLENGKKLKGVNELVN |
| | | EYRQKNKNEKVSFLKLLDKQILSEKEKLSFGIENDEQLLVVLNSFYETAE |
| | | EKTKILRTLFGDFVEHNENYDLDKTYISKVAFNTISHKWTNETHKFEELL |
| | | YGAMKEDKPIGLNYDKKEDSYKFPDFIALGYLKKCLNNLDCDTKFWKE |
| | | KYYENNADKKDKDKGFLTGGQNAWDQFLQIFIFEFNQLFNSEAFDNK |
| | | GKEIKIGYDNFRKDFEEIINQKDFKNDENLKIAIKNFADSVLWIYQMAK |
| | | YFAIEKKRGWDDDFELSEFYTNPSNGYSLFYDRAYEEIVQKYNDLRNYL |
| | | TKKPYKEDKWKLNFENPTLANGFDKNKESDNSTVILRKKRKYYLGLMK |
| | | KGNNKIFEDRNKAEFIRNIESGAYEKMAYKYLPDVAKMIPKCSTQLNE |
| | | AKNHFRNSADDLEIKKSFSNPLKITKRIFDLNNIQYDKTNVSKKISGDNK |
| | | GIKIFQKEYYKISGDFDVYKSALNDWIDFCKDFLSKYDSTKDFDFSILRKT |
| | | KDYKSLDEFYVDVAKITYKISFTPVSESYIDQKNKNGELYLFEIYNQDFAK |
| | | GKMGAKNLHTLYFENVFSPENISKNFPIKLNGNAELFFRPKSIESKKEKR |
| | | NFVREIVNKKRYSEDKIFFHCPITLNRETGSIYRFNNYVNNFLSENNINII |
| | | GVDRGEKHLAYYSVIDKNGVKIGGGSFNEINKVDYAKKLEERAGEREQ |
| | | SRKDWQVVEGIKDLKKGYISQVVRELADLAIKHNAIIVLEDLNMRFKQI |
| | | RGGIEKSIYQQLEKALIDKLSFLVEKGEKDPNQAGHILKAYQLAAPFTSF |
| | | KDMGKQTGIVFYTQASYTSKTCPNCGFRKNNNKFYFENNIGKAQDAL |
| | | KKLKTFEYDSENKCFGLSYCLSDFANKEEVEKNKNKKRNNAPYSDIEKK |
| | | DCFELSTKDAVRYRWHDKNTERGKTFFEGESVYEEKEEKEIGQ.TKRGL |
| | | VKEYDISKCLIGLFEKTGLDYKQNLLDKINSGKFDGTFYKNLFNYLNLLFE |
| | | IRNSISGTEIDYISCPECQFHTDKSKTIKNGDDNGSYNIARKGMIILDKIK |
| | | QFKKENGSLDKMGWGELFIDLEEWDKFAQKKNNNIIDK |
| 13 | Cas12 | MKSFDSFTNLYSLSKTLKFEMRPVGNTQKMLDNAGVFEKDKLIQKKY |
| | | GKTKPYFDRLHREFIEEALTGVELIGLDENFRTLVDWQKDKKNNVAM |
| | | KAYENSLQRLRTEIGKIFNLKAEDWVKNKYPILGLKNKNTDILFEEAVFG |
| | | ILKARYGEEKDTFIEVEEIDKTGKSKINQISIFDSWKGFTGYFKKFFETRK |
| | | NFYKNDGTSTAIATRIIDQNLKRFIDNLSIVESVRQKVDLAETEKSFSISL |
| | | SQFFSIDFYNKCLLQDGIDYYNKIIGGETLKNGEKLIGLNELINQYRQNN |
| | | KDQKIPFFKLLDKQILSEKILFLDEIKNDTELIEALSQFAKTAEEKTKIVKKL |
| | | FADFVENNSKYDLAQIYISQEAFNTISNKWTSETETFAKYLFEAMKSGK |
| | | LAKYEKKDNSYKFPDFIALSQMKSALLSISLEGHFWKEKYYKISKFQEKT |
| | | NWEQFLAIFLYEFNSLFSDKINTKDGETKQVGYYLFAKDLHNLILSEQID |
| | | IPKDSKVTIKDFADSVLTIYQMAKYFAVEKKRAWLAEYELDSFYTQPDT |
| | | GYLQFYDNAYEDIVQVYNKLRNYLTKKPYSEEKWKLNFENSTLANGW |
| | | DKNKESDNSAVILQKGGKYYLGLITKGHNKIFDDRFQEKFIVGIEGGKY |
| | | EKIVYKFFPDQAKMFPKVCFSAKGLEFFRPSEEILRIYNNAEFKKGETYSI |
| | | DSMQKLIDFYKDCLTKYEGWACYTFRHLKPTEEYQNNIGEFFRDVAED |
| | | GYRIDFQGISDQYIHEKNEKGELHLFEIHNKDWNLDKARDGKSKTTQK |
| | | NLHTLYFESLFSNDNVVQNFPIKLNGQAEIFYRPKTEKDKLESKKDKKG |
| | | NKVIDHKRYSENKIFFHVPLTLNRTKNDSYRFNAQINNFLANNKDINII |
| | | GVDRGEKHLVYYSVITQASDILESGSLNELNGVNYAEKLGKKAENREQ |
| | | ARRDWQDVQGIKDLKKGYISQVVRKLADLAIKHNAIIILEDLNMRFKQ |
| | | VRGGIEKSIYQQLEKALIDKLSFLVDKGEKNPEQAGHLLKAYQLSAPFET |
| | | FQKMGKQTGIIFYTQASYTSKSDPVTGWRPHLYLKYFSAKKAKDDIAK |
| | | FTKIEFVNDRFELTYDIKDFQQAKEYPNKTVWKVCSNVERFRWDKNL |
| | | NQNKGGYTHYTNITENIQELFTKYGIDITKDLLTQISTIDEKQNTSFFRD |
| | | FIFYFNLICQIRNTDDSEIAKKNGKDDFILSPVEPFFDSRKDNGNKLPEN |
| | | GDDNGAYNIARKGIVILNKISQYSEKNENCEKMKWGDLYVSNIDWDN |
| | | FVTQANARH |
| 14 | Cas12 | MNTQKKEFNPKSFKDFTNLYSLNKTLRFSLTPNKKTAEILEFNKQKEVK |
| | | CFSNDRKIAGAYQEIKKYLNKLHQEFIQEAMKFFAFSEEELKGFEKEYLN |
| | | LLNFTDKDNFKKKNKIRNEYEQERKILTIKIATYFSKFKSEKYQSFNLANI |
| | | TGKKVFSILEQKYKEDKKTLKIIHIFKYKPTKDEKKEGEAVNFSTYLTGFN |
| | | ENRKNFYKSEDKAGQFATRTIDNLAQFIKNKKLFEDKYQKNYSKIGILD |
| | | EQIKIFNLDYFNNLFLQEGLDEYNGILGNNKGEENKSNEGINQKINIFK |
| | | QKEKARLKKEKENFNKSDFPLFKELYKQIGSIRKENDVYVEIKTDKELVE |
| | | ELNNFPKNVENYLKDIQSFYKTFFEKLQNEEYELDKIYLPKSVGTYFSYIA |
| | | FSDWNKLAFIYNKRYKNEKIKIVEGGDVNVQYRSLEVLKNRIDELKDED |
| | | NLNFNKFFIDKLKFNEAKKENNWQNFWFCIEYYINSQFIGGEKNILNKE |
| | | KNEYEILPFGSLKELKEKYFEAVKKYKEKMVDTESGLTDDEEKEIKETLK |
| | | NYLDRIKEIERIAKYFDLKKSFEEIKQEDLDSNFYGEYQKVVDKTNELKIY |
| | | QYYSEFRNYLTQNNSVEEKIKLNFNSGLLLDGWDLNKEKVKFSIIFQEN |
| | | GKYYLGIINKEKDKTILDKDKHPEIFTKNSDFRKMEYKLFPSPSKMLPKIS |
| | | FSETAKKGDEDVGWSEEIQKIKDEFAEFQEYKKKSKDNWKDEFNRGK |
| | | LNKLIDYYKQVLEKHSEGYMNTYNFELKDSSKYKNLGEFNDDIARQNY |
| | | KVKFVGIDKNYIDEKVANGELFLFQIYNKDFSEDKKEGSTNNLETIYFKE |
| | | LFSKENLENPVFKLSGGAEMFFRNKIEKKKEKKKLDKDGKPMISKKGEK |
| | | VVDKRRFSENKILFHLPIEINYGKGKMPNFNKKINEYISKNPENIKIIGID |
| | | RGEKHLLYYSIIDQNGNNIESMSLNAVDEFGNFVNPEKLEEYEIDNNG |
| | | KKERRWKYIVNDKEIKVTNYQRKLDELEKERQKSRQSWQNINKIKNLK |
| | | KGYISFVVKKIVDLAIENNAIIILEDLNFGFKSFRQKIEKNVYQQFEKALID |
| | | KLGFVVDKQKQNQRFAPQLSAPFESFQKIGKQTGIVYYVLANNTSKVC |
| | | PSCQWIKNFYLKYEKKNTIFNLQKNQKLKVFFEQEKNRFRFEYQMSKE |
| | | YISVYSDVDRQRYDKTKNQNKGGYLEYKNSNQKEIIDKDGVIQKQSITL |
| | | QLKELFKENHIDLEKEILKQLDNKKEKNSGYTGVYNKFIYLFNLILQIRNA |
| | | ISFREKDYIQCPSCHFDTRKENYLKINDGDGNGAYNIALRGLYLLKGKN |
| | | GIINNLEKIKLIFSNNDYFQWAKKLKNKK |
| 15 | Cas12 | MEEKMLKSYDYFTKLYSLQKTLRFELKPIGKTLEHIKNSGIIESDETLEEQ |
| | | YAIVKNIIDKLHRKHIDEALSLVDFTKHLDTLKTFQELYLKRGKTDKEKEE |
| | | LEKLSADLRKLIVSYLKGNVKEKTQHNLNPIKERFEILFGKELFTNEEFFLL |
| | | AENEKEKKAIQAFKGFTTYFKGFQENRKNMYSEEGNSTSIAYRIINENL |
| | | PLFIENIARFQKVMSTIEKTTIKKLEQNLKTELKKHNLPGIFTIEYFNNVLT |
| | | QEGISRYNTIIGGKTTHEGVKIQGLNEIINLYNQQSKDVKLPILKPLHKQI |
| | | LSEEYSTSFKIKAFENDNEVLKAIDTFWNEHIEKSIHPVTGNKFNILSKIE |
| | | NLCDQLQKYKDKDLEKLFIERKNLSTVSHQVYGQWNIIRDALRMHLE |
| | | MNNKNIKEKDIDKYLDNDAFSWKEIKDSIKIYKEHVEDAKELNENGIIK |
| | | YFSAMSINEEDDEKEYSISLIKNINEKYNNVKSILQEDRTGKSDLHQDKE |
| | | KVGIIKEFLDSLKQLQWFLRLLYVTVPLDEKDYEFYNELEVYYEALLPLNS |
| | | LYNKVRNYMTRKPYSVEKFKLNFNSPTLLDGWDKNKETANLSIILRKN |
| | | GKYYLGIMNKENNTIFEYYPGTKSNDYYEKMIYKLLPGPNKMLPKVFFS |
| | | KKGLEYYNPPKEILNIYEKGEFKKDKSGNFKKESLHTLIDFYKEAIAKNED |
| | | WEVFNFKFKNTKEYEDISQFYRDVEEQGYLITFEKVDANYVDKLVKEG |
| | | KLYLFQIYNKDFSENKKSKGNPNLHTIYWKGLYDSENLKNVVYKLNGE |
| | | AEVFYRKKSIDYPEEIYNHGHHKEELLGKFNYPIIKDRRYTQDKFLFHVPI |
| | | TMNFISKEEKRVNQLACEYLSATKEDVHIIGIDRGERHLLYLSLIDKEGNI |
| | | KKQLSLNTIKNENYDKEIDYRVKLDEKEKKRDEARKNWDVIENIKELKE |
| | | GYMSQVIHIIAKMMVEEKAILIMEDLNIGFKRGRFKVEKQVYQKFEK |
| | | MLIDKLNYLVFKNKNPLEPGGSLNAYQLTSKFDSFKKLGKQSGFIFYVP |
| | | SAYTSKIDPTTGFYNFIQVDVPNLEKGKEFFSKFEKIIYNTKEDYFEFHCK |
| | | YGKFVSEPKNKDNDRKTKESLTYYNAIKDTVWVVCSTNHERYKIVRNK |
| | | AGYYESHPVDVTKNLKDIFSQANINYNEGKDIKPIIIESNNAKLLKSIAEQ |
| | | LKLILAMRYNNGKHGDDEKDYILSPVKNKQGKFFCTLDGNQTLPINAD |
| | | ANGAYNIALKGLLLIEKIKKQQGKIKDLYISNLEWFMFMMSR |
| 16 | Cas12 | MNNYDEFTKLYPIQKTIRFELKPQGRTMEHLETFNFFEEDRDRAEKYKI |
| | | LKEAIDEYHKKFIDEHLTNMSLDWNSLKQISEKYYKSREEKDKKVFLSE |
| | | QKRMRQEIVSEFKKDDRFKDLFSKKLFSELLKEEIYKKGNHQEIDALKSF |
| | | DKFSGYFIGLHENRKNMYSDGDEITAISNRIVNENFPKFLDNLQKYQE |
| | | ARKKYPEWIIKAESALVAHNIKMDEVFSLEYFNKVLNQEGIQRYNLALG |
| | | GYVTKSGEKMMGLNDALNLAHQSEKSSKGRIHMTPLFKQILSEKESFS |
| | | YIPDVFTEDSQLLPSIGGFFAQIENDKDGNIFDRALELISSYAEYDTERIYI |
| | | RQADINRVSNVIFGEWGTLGGLMREYKADSINDINLERTCKKVDKWL |
| | | DSKEFALSDVLEAIKRTGNNDAFNEYISKMRTAREKIDAARKEMKFISE |
| | | KISGDEESIHIIKTLLDSVQQFLHFFNLFKARQDIPLDGAFYAEFDEVHSK |
| | | LFAIVPLYNKVRNYLTKNNLNTKKIKLNFKNPTLANGWDQNKVYDYAS |
| | | LIFLRDGNYYLGIINPKRKKNIKFEQGSGNGPFYRKMVYKQIPGPNKNL |
| | | PRVFLTSTKGKKEYKPSKEIIEGYEADKHIRGDKFDLDFCHKLIDFFKESIE |
| | | KHKDWSKFNFYFSPTESYGDISEFYLDVEKQGYRMHFENISAETIDEYV |
| | | EKGDLFLFQIYNKDFVKAATGKKDMHTIYWNAAFSPENLQDVVVKLN |
| | | GEAELFYRDKSDIKEIVHREGEILVNRTYNGRTPVPDKIHKKLTDYHNG |
| | | RTKDLGEAKEYLDKVRYFKAHYDITKDRRYLNDKIYFHVPLTLNFKANG |
| | | KKNLNKMVIEKFLSDEKAHIIGIDRGERNLLYYSIIDRSGKIIDQQSLNVI |
| | | DGFDYREKLNQREIEMKDARQSWNAIGKIKDLKEGYLSKAVHEITKM |
| | | AIQYNAIVVMEELNYGFKRGRFKVEKQIYQKFENMLIDKMNYLVFKD |
| | | APDESPGGVLNAYQLTNPLESFAKLGKQTGILFYVPAAYTSKIDPTTGF |
| | | VNLFNTSSKTNAQERKEFLQKFESISYSAKDGGIFAFAFDYRKFGTSKTD |
| | | HKNVWTAYTNGERMRYIKEKKRNELFDPSKEIKEALTSSGIKYDGGQN |
| | | ILPDILRSNNNGLIYTMYSSFIAAIQMRVYDGKEDYIISPIKNSKGEFFRT |
| | | DPKRRELPIDADANGAYNIALRGELTMRAIAEKFDPDSEKMAKLELKH |
| | | KDWFEFMQTRGD |
| 17 | Cas12 | MASSHFISLDNSFSKFTNLYSLSKTLRFELVPTENTTVMLENNNVFKKD |
| | | QIIQVKYEKTKPFIDRLHREFIKEALSNYAVSGLQEYFEILRAGGKKANLD |
| | | SAKKQLRKHVVDQFNATASLWVSRHKDVGFKGEGIELLFKEAVFKLLK |
| | | EKYGTDMNALIEDNHGKQISIFDSWKGFTGYFDKFQQTRRNLYKDDG |
| | | KEGRVATRIIDQNLTRFCDNIFVYEKIKDKVSFIDVEKSFGKTCSEVFIPD |
| | | YYNTCLLQDGIDSYNEFIGGKPLENGEKVQGLNELINLYRQTTGDKVPY |
| | | FKKLEKQILGEKDEVFIDEITDEDFVPRVLAFYRTVDAKYKLFLKLLDDFV |
| | | TNQDVYELSQIYISKKGLQEKLYRWLTPSAREVYDEELFEVLKKAKKVN |
| | | NKDKQKVSGYVPDFVEVLYIKQALENIDAKLIWSDRYYSDGENEGIIDK |
| | | GFSSWKQFLVILNHEYRQLLSFEDHVIIDKELDFDKEVKQLTDTVEIVSQ |
| | | DKNARTVTYRGGYDVYKAKLAELGQSFEKDTCTKKVIKNFADSVLSMY |
| | | HFAMMFAVWDDTYPLDVFYTNNEFGYLLYYEDAYKNIVQEYNKLRN |
| | | YLTKKPYSTEKWKLNFENPTLAAGFDKNKESDNSTVILRQGDKYFLGV |
| | | MKKGFNKlFDNSQISQTGNSPEAYFEKMVYKYTKDVVTGIPKSSTQVK |
| | | EVQEHFRNSDEDFFLEECSSVGNFIVPLKITKEIFDLNNKVYAKEDISQA |
| | | MYRWALNTDEEKNYVKSFQKSYLSLGGSPELYCKSVTLWIGFCLNFLK |
| | | SYPSAAYFDYSQLRQASDYESVDECYQELNNAGYTILFQNVSEKYVRV |
| | | KNKNGELYLFQIKNKDWNEGSTGKKNLHTLYFESLFSKENAKQGFPFK |
| | | LSGNAELFFRPGSIEQTYERRNFPREIPLKRRYSKDGIFFHIPVQVNRTK |
| | | VGSPNQFNKEVNDFLAGNPNINIIGVDRGEKHLVYYSVISQNGEKIDG |
| | | GSFNEINGQDYHDKLEKRAKEREQQRRDWETVEGIKDLKKGYISQVV |
| | | KKLADLAIEHNAIIVMEDLNMRFKQIRGGIEKSVYQQLEKALIDKLSFLV |
| | | NKGEVDPQKAGHLLKAYQLTAPIDAFKDMGKQTGIMFYTQAAYTSKI |
| | | DPVTGWRPHLYLKYSSVEKAKDDISRFTKIAYKNDRFEFTYNITDFRTQ |
| | | KEWPLKTEWTVCSCVERFRWNKKLANGKGDYEHYPNVTDDFKKLFD |
| | | SVGINYLQENIKSQVVNLDENTNVEFFREFIKLFALVCQIRNTNSEEAG |
| | | NLNDFILSPVEPFFDSRSAEDFGKGLPSNGDENGAYNIARKGMIILNTL |
| | | STFKNDHGSCEGLSWGDLYISDTQWDDFAQSFHG |
| 18 | Cas12 | MDAKEFTGQYPLSKTLRFELRPIGRTWDNLEASGYLAEDRHRAECYPR |
| | | AKELLDDNHRAFLNRVLPQIDMDWHPIAEAFCKVHKNPGNKELAQD |
| | | YNLQLSKRRKEISAYLQDADGYKGLFAKPALDEAMKIAKENGNESDIE |
| | | VLEAFNGFSVYFTGYHESRENIYSDEDMVSVAYRITEDNFPRFVSNALI |
| | | FDKLNESHPDIISEVSGNLGVDDIGKYFDVSNYNNFLSQAGIDDYNHII |
| | | GGHTTEDGLIQAFNVVLNLRHQKDPGFEKIQFKQLYKQILSVRTSKSYI |
| | | PKQFDNSKEMVDCICDYVSKIEKSETVERALKLVRNISSFDLRGIFVNKK |
| | | NLRILSNKLIGDWDAIETALMHSSSSENDKKSVYDSAEAFTLDDIFSSVK |
| | | KFSDASAEDIGNRAEDICRVISETAPFINDLRAVDLDSLNDDGYEAAVS |
| | | KIRESLEPYMDLFHELEIFSVGDEFPKCAAFYSELEEVSEQLIEIIPLFNKA |
| | | RSFCTRKRYSTDKIKVNLKFPTLADGWDLNKERDNKAAILRKDGKYYLA |
| | | ILDMKKDLSSIRTSDEDESSFEKMEYKLLPSPVKMLPKIFVKSKAAKEKY |
| | | GLTDRMLECYDKGMHKSGSAFDLGFCHELIDYYKRCIAEYPGWDVFD |
| | | FKFRETSDYGSMKEFNEDVAGAGYYMSLRKIPCSEVYRLLDEKSIYLFQI |
| | | YNKDYSENAHGNKNMHTMYWEGLFSPQNLESPVFKLSGGAELFFRK |
| | | SSIPNDAKTVHPKGSVLVPRNDVNGRRIPDSIYRELTRYFNRGDCRISD |
| | | EAKSYLDKVKTKKADHDIVKDRRFTVDKMMFHVPIAMNFKAISKPNL |
| | | NKKVIDGIIDDQDLKIIGIDRGERNLIYVTMVDRKGNILYQDSLNILNGY |
| | | DYRKALDVREYDNKEARRNWTKVEGIRKMKEGYLSLAVSKLADMIIE |
| | | NNAIlVMEDLNHGFKAGRSKIEKQVYQKFESMLINKLGYMVLKDKSID |
| | | QSGGALHGYQLANHVTTLASVGKQCGVIFYIPAAFTSKIDPTTGFADLF |
| | | ALSNVKNVASMREFFSKMKSVIYDKAEGKFAFTFDYLDYNVKSECGRT |
| | | LWTVYTVGERFTYSRVNREYVRKVPTDIIYDALQKAGISVEGDLRDRIA |
| | | ESDGDTLKSIFYAFKYALDMRVENREEDYIQSPVKNASGEFFCSKNAG |
| | | KSLPQDSDANGAYNIALKGILQLRMLSEQYDPNAESIRLPLITNKAWLT |
| | | FMQSGMKTWKN |
| 19 | Cas12 | MVNKQNERGDFDDLTNLYEISKTLRFELVPVGETDRMLKEENVFKVD |
| | | ENIKRKYQQTKLFFDRIHREFAKEALSVEGILSELEEYLAIFIEWRKDKKI |
| | | HEKTLNQKEKELRKQVVSAFNAMANKWIERYGDVNLKKKNVEFLFEE |
| | | GIFRVLKERYGEEDGSTITASDTGEVFSIFDSWKGFTGYFAKFFETRKNF |
| | | YKDDGTATAIATRIVDENLRRFCDNLIVAQRLTENIDFSEVENNFQIKIK |
| | | EVLFMEFYNKCLLQDDIDFYNKVIGGETLKTGEKLKGINELVNLHRHKT |
| | | GEKLPFLKTLDKQILGRKEQFLDEIESEEELLEKLKDFQNVATKKIKVIKSL |
| | | FGDFVENNENYDLEKIYISKKAFNTISRKWTGETEQFEKLLFESMKSDK |
| | | PAGLKYDKKENNYKFPDFIAVSYIKDALENFSGEQKFWKDRYYIELELD |
| | | NQVVWKQFLDIFYWEFSSLFKRSFVNKETGEISEVGCDIFEKKFINLIDD |
| | | FEYNQKSKILIKDFADSVLSVYQMANYFSLEKKRKWSTEFETDSKFYDD |
| | | SEIGFRNCFYEDVFEGIVQ.VYNKLRNYLTKKPFSEEKWKLNFENPTLAA |
| | | GWDKNKEKDNSTVILRKDEKYFLAIMKKGNNVIFDDRNKALFSQNLE |
| | | HGKYEKWYKFAKDVTLGIPKSTTQTKSVIAHFKNSDEDYQITNGSAVG |
| | | DFLEPLVVTKRIFELNNKIYSKNNLGKVLYRSEVSKDKQKEYIKLFQKKYL |
| | | VLGGNKNLYRDAVKEWIDFCKSFIKVYPSYKYFDFSLLKEAVEYNSVDE |
| | | FYKELNSYGYAISFQDISCDYIEEKNKNGELYLFQIKNKDWNKGSTGMK |
| | | NLHTLYFESLFSEENIKNNFVTKLNGGAEIFYRPKTSKEKLGRKKIVRNG |
| | | QEVFVVNHKRYSEDKIFFHCSIALNRGKGKLLKFNARINDLLANNPDIN |
| | | VIGVDRGEKHLAYYSIIDQKCKILDSGTLNEVGAKVDYHEKLSNRAKKR |
| | | EDGRRDWGWGQIEDIKNLKKGYVSQVVHKLAELIIKYNAILVFEDLN |
| | | MRFKQIRGGIEKSIYQQLEKALIDKLNFLVKKGEKDSKSAGHLLKAYQL |
| | | AAPFETFDKMGKQTGVIFYTQASYTSKIDPITGWRPNLYLKHSNANDS |
| | | QKKIAKFSRIEFINDRFEFEYDLKKFIEMKEVPENTKWTLCSCVQRYRW |
| | | NRKLNANKGGYDSYNDLTKNFKALFESVGIDIKKNIKEQIVKMEIKGNE |
| | | KFFKSFIFYWQLLCQIRNTDELKKGDDNDFILSPVEPFFDSRKKNGDDL |
| | | PKNGDDNGAYNIARKGVIVLNKISEFSKQNGNCEKCGWKELYVSAKD |
| | | WDDFVQAK |
| 20 | Cas12 | MQNKQSFADFTNLYSLSKTLRFELKPIGQTQAMLDENKIFEVDENRKK |
| | | AYDKTKPYFDRLHREFINESLSNAQLKGISEYFETFKQFRSNQNNKDLK |
| | | ELINKQQKFLRHQIVTLFDENGKHWATTKYAHLKIKKKNLDILFDEQVF |
| | | YILKERYGSEKETQLVDKETGAVTSIFDNWKGFTGYFTKFFETRKNFYK |
| | | SDGTSTALATRIIDQNLNRFFDNLETFHKIKDKIDVKEVEIFFKLKADNV |
| | | FSIDFYNQCLLQNGIDKYNDFLGGQTLENGEKQKGINEIINKYRQDNK |
| | | DQKLPFLKKLDKQLSEKDRFINEIESKEEFFQVLTEFYQSATVKVTIIKTL |
| | | LNDFVHNTDKYKLEKIYLTKEAFNTIANKWTDETQIFEDNLDLVLKNKK |
| | | ITAKQDFIPLAYIKEALEVIEKDRKFFKDRYYNDPQIGFFPDQSYWEQFL |
| | | AILNFEFMTHFQRVAKDKITGKKIELGYFVFEKRIKELLDSDPSLNSQSKI |
| | | IIKEFADEVLHIFQMAKYFALEKKREWKGDYYQLDDQFYNHIDYGFKD |
| | | QFYENAYEKIVQPYNKIRNYLTKKPYSDVKWKLNFGNPTLANGWDKN |
| | | KEADNTAVILKKDGNYYLGVMKKGKNKIFSDQNKEKYKAYNSAYYEKL |
| | | VYKLFPDPSKMFPKVCFSKKGLNFFQPSEEILRIYKNNEFKKGNTFSISS |
| | | MQKLIAFYIDCLGLYEGWKHYEFKNIKDVRQYKENIGEFYADVAESGY |
| | | KLWFEKISEEYITQKNQLGELFLFQIYNKDFAKKTTGRKNLHTIYFEELFS |
| | | QTNIDNNFPFKLNGQAELFYRPKSLEKIEEKRNFKRSIVNKKRYTQNKIF |
| | | FHVPITLNRTSENIGRFNVRVNNFLANNSNVNIVGVDRGEKNLAYYSII |
| | | KQNGEVLKSGSLNIINGVDYHALLTDRAQRREQERRNWQDVESIKDL |
| | | KRGYISQVVHELVSLAIKYNAIIVMEDLNMRFKQIRGGIEKSTYQQLEK |
| | | ALIEKLNFLVNKEETDSNQAGNLLNAYQLTAPFKTFKDMGKQTGIIFYT |
| | | QASYTSKIDPLTGWRPNIYLRYSNAKQAKADILMFTNIYFSEKKDRFEF |
| | | TYDLEKIDDKRKDLPIKTEWTVCSNVERFSWEKSLNNNKGGYVHYPIQ |
| | | DSNGEESITSKLKKLFMDFGIDLTDIKTQIESLDTNKKDNANFFRKFIFYF |
| | | QLICQIRNTQVNKSDDGNDFIFSPVEPFFDSRFADKFRKNLPKNGDEN |
| | | GAYNIARKGLIILHKISDYFVKEGSTDKISWKDLSISQ.TEWDNFTTDK |
| 21 | Cas12 | MDKQKNKLQNFTNLYELSKTLRFELKPVGETQHLLEENKVFGIDGNIK |
| | | KKYEATKPFFDRLHRKFVKEALVNIALGGLDNYLEVYKKFTNDRKDKEN |
| | | QKELEKQEKLLRKQIKIFFDSQANQWKEKYNKINFKKSGLNILFEESIFQ |
| | | LLKEIYGKEDDAFLKNDDNEFIFDKDGNKISIFDSWKGFTGYFKKFFETR |
| | | KNFYKDDGTSTAIATRIIDQNLRRFCDNIFIYNKIKNKLDFSSLEKEQDV |
| | | VLEEIFTTAYYMDCILQDDIDLYNGVLGGETLDDGTKIKGLNEIINKYRQ |
| | | DNKGDKIPFFKKLDKQILSEKDRKFLDEIESEEELAELLKIFINNTEAKVKV |
| | | FDELVNQLCVNDSDFELDKIYISKEAFNTISHKWTNQTHEFERVLFEEM |
| | | KPDKITGLDYKKAEDKYKFPDFIALKYIIKSLNTLDKDSEFWKSHYYKTEE |
| | | NQNAILSLEEKVGEQFLQIYKYELQRLHSRNVNVENKDGKMKEKEIGL |
| | | DYSLTTVKELLKNFKLTDKSKIIIKDFADNVLQYYQLAKYFSVEKNREWN |
| | | YTKLELADFYINPDFGYEIFYGNAYEEIIQJYNKLRNYLTKKPFSEEKWKL |
| | | NFENPTLAGGWDKNKERGNATVILRKNEKYYLGIMAKGYNDIFTDKN |
| | | KDKFDGEGYEKMVYKLFPGPNKMMPKVCFSKKGLDFFEPSEKIIDIYK |
| | | DGKFKQGDTFSIDSMQQLIDFYKRALREYNGWKMYDFSKLKDTNDYT |
| | | TNIGEFYNDVACAGYKVWFDNISEEYIQEKNENGELYLFEIHNKDWNL |
| | | KDEKKKTGTKNLHTLYFESLFSDENALRDFVMKLSGEAELFFRPKTNAD |
| | | KLGYRKDKKGNKVVKNKRYSEDKMFLHLSINLNRGKGQAFWFNRNIN |
| | | NFLANNSDINVIGIDRGEKHLAYYSVISQQGEILDNGSLNEIAGVDYYA |
| | | KLSKRAKEREGQRKDWQAVSDIKNLKKGYISQVVRKLADLAIEHNAIIV |
| | | LEDLNMRFKQIRGGIEKSIYQQLEKALIEKLNFLVNKKEIDSDKAGNLLR |
| | | AYQLTAPFETFQKMGKQTGIIFYTQASYTSKIDPLTGWRPNLYLKKGN |
| | | AKINKEQIEKFSKIEFTNNRFEITYDLKNFGDKKKKYPQKTKWTLCSSVE |
| | | RWRWDRKLNNNKGGYIHYEDLTTEFKSLFEKFEIDIEGDILEQIKTIDEN |
| | | DRNNARLFSGFIYLWGLLSQIRNTDGELDEKIKKLEREDKNEEISEKEKF |
| | | DVDFILSPVEPFFDSRTPEKFGENLPKNGDDNGAYNIARKGHTLERIKK |
| | | FYELSDKEREKLKYPDLFITNAEWDDFATKRDS |
| 22 | Cas13 | MDNNTTLEKTELGLGITYNHDKVEDKHYFGGFFNLAQNNIDLVAQEF |
| | | KKRLLVQGKDSINIFSNYFSDQCSITNLERGIKVLSEYFPVIFYFDLDENN |
| | | KSKSIRQH11LLLDTINNLRNYYTHYYHKKVIIDDALYPLLDTILLKVVLEIK |
| | | KKKLKEDKTKQLLKKGLEKEMAILFNLMKKEQKEKKIKGWNIDKNIKG |
| | | AVLNRAFSHLLYNDGISDYRKSKSNTEDENLKDTLSESGILFLLSFFLNKK |
| | | EQEQLKANIKGYKGKIASIPDEEITLKNNSLRNMATHWTYSHLTYKGLK |
| | | HRIKTDHEKETLLVNMVDYLSKVPNEIYQNLSEQNKSLFLEDINEYMR |
| | | DNEENNDSSEASRVIHPVIRKRYENKFAYFAIRFLDEFAEFPTLRFMVN |
| | | VGNYIHDNRKKDIGGTSLITNRTIKQQINVFGNLTEIHKKKNDYFEKEE |
| | | NKEKILEWELFPNPSYHFQKENIPIFIDLEKSKETNELAKEYAKEKKKIFG |
| | | SSRKKQQNTAKKNREAIINLVFDKYKTSDRKTVTFEQPTALLSFNELNA |
| | | FLYAFLVENKTGKELEKIIIEKIANQYQILKNCSSTVDKTNDSIPKSIKKIA |
| | | HPTTDSFYSEGKKIDIEKLERDIKIEIEKTNEKLETIKENETSAKNYKRNER |
| | | DIQKRKLYRKYVFFTNEIGIEATWITNDILRFLDNKENWKGYQHSELQK |
| | | FISQYDNYKKEALGLLESEWNLESEAFFGQKLKRIFQSNFTFETFYKKYL |
| | | DNRKDTLETYLSAIENLKTMTDVPPKILKKSWAELFRFFDKKIYLLSTIET |
| | | KINELITKPINLSRGVFDEKPTFINGKSPNKENDQHLFANWFIHAKEQTI |
| | | FQDFYNLALETPKEINNLKKQNYKLERSINNLKIEDIYIKQMVDFLYQKL |
| | | FEQSFKGSLQDLYTSKEKREVEKSKAKNEQ.TPDESFIWKKQVEINALN |
| | | GRIIAKTKIKDIGKFKNLLTDNKITHLISYDNRIWNFSLDNDGDTTKKLYS |
| | | LNTELESYERIRREKLLKQIQEFEQFLLKQETEYSAERKHPEKFEKDGNP |
| | | NFKKYIIEGMLNKITPVNEIEELEILKSKEDVFKIDFNEIVKLNNESIKKGY |
| | | LLIMIRNKFAHNQLIDKNLFTFSLQLYSKNENENFSEYLDKVCQKIIQEFI |
| | | EKLK |
| 23 | Cas13 | MNETDYLAKRLEYNYASIEDKHYFGGYFNLAQNNINDLSKAFKEKFG |
| | | MKPKSCILDFFTQDKAIAEYQLGVEFLQKNLPVIRYLYLPTSHKRFENVP |
| | | KNQLISEQRNYFKNSLKVLKNLIRDYRNFYTHHFHKPIPVFPETYKLLDD |
| | | LFLAVANDVKKHRMKTDASKQLLKKGLIEELAQLEKLKLEDLKKLKREG |
| | | KKVNLNDKEAITNAILNDSFSHLLPKENTISKYYSAVPTEDIDTENGVTIS |
| | | ESGIIFLLGLFLTKKQSEDLRSRVKGFKAKLIVNPENPINKKNNSLKYMA |
| | | THWVFGYLGFKGLKNRFTTTFTKDTLLAQIVDELSKVPDELYQVLPEEL |
| | | KNEFLEDMNEYLKEENSESLDKATVIHPVIRKRYENKFAYFALRFLDEFV |
| | | DFPTLRFQLHLGNYVHDKREKPIEGTKYVTERIVKEKIKAFAKLSEAAQL |
| | | KQKYFEEKENHQSIGLQLYPNPSYNFVGNNIPIHLNLNEHFFPKEVKIV |
| | | AGRLKKRNSSYKSDHPEEYKVRTDNKIKPDAILQDLGKPEKLAPVAML |
| | | SLNELPALLHLVLTKKTPEEIEIIIAQKIAERYNVLTNYKAGDDISKGQITK |
| | | NLLKAKQKKEVNLDKLQLAIEKEIAVTNDKLQTIALHIKERNDPKQKRK |
| | | YVFTNKEIGLQVTWLANDLKRFMPKGSRQNWRGQHHSQLQKSLAFY |
| | | DIQPKEPLSLLEEVWDFKNEAYLWNNGIRRSFDKRDFISFYTSYLNNRK |
| | | ETFQRFKDQLNGIRSNKKILDKFIKQQHLWNLFHKRLYVIDTIEEQVEKL |
| | | LVKPMQFPKGVFDHKPTYIKGKSIQENPECFADWYVAWNQHTDYQK |
| | | FYSWDRDYKSAYLSGEQEKTEKRFIRVQGSKINKVKQQDVLLAKMASII |
| | | FNELYLPEDAEHLDLNLSDIYKTQTERKAEIEAALIQSHKTTGDNSANIIK |
| | | STSAWTLTVPYCSKNIYEPQVKLKELGKFKKFIASQKVQTLFEYKPQKI |
| | | WNKTELEEVLELKANSYEVIRRDYLLKSIQEFEKYMIKKLPTLIDTNEHP |
| | | NFNKYLTTFLKSLELVSEEDAKWLISKKDFDTTPIDELKKQSKIMEKAFLL |
| | | VMIRNKFSHNQLPRKIYYDEIYKNVPNAVSINFNELFLEYTNQTILEFK |
| 24 | Cas13 | MESIIGLGLSFNPYKTADKHYFGSFLNLVENNLNAVFAEFKERISYKAK |
| | | DENISSLIEKHFIDNMSIVDYEKKISILNGYLPIIDFLDDELENNLNTRVKN |
| | | FKKNFIILAEAIEKLRDYYTHFYHDPITFEDNKEPLLELLDEVLLKTILDVK |
| | | KKYLKTDKTKEILKDSLREEMDLLVIRKTDELREKKKTNPKIQHTDSSQIK |
| | | NSIFNDAFQGLLYEDKGNNKKTQVSHRAKTRLNPKDIHKQEERDFEIP |
| | | LSTSGLVFLMSLFLSKKEIEDFKSNIKGFKGKVVKDENHNSLKYMATHR |
| | | VYSILAFKGLKYRIKTDTFSKETLMMQMIDELSKVPDCVYQNLSETKQK |
| | | DFIEDWNEYFKDNEENTENLENSRVVHPVIRKRYEDKFNYFAIRFLDEF |
| | | ANFKTLKFQVFMGYYIHDQRTKTIGTTNITTERTVKEKINVFGKLSKMD |
| | | NLKKHFFSQLSDDENTDWEFFPNPSYNFLTQADNSPANNIPIYLELKN |
| | | QQIIKEKDAIKAEVNQTQNRNPNKPSKRDLLNKILKTYEDFHQGDPTAI |
| | | LSLNEIPALLHLFLVKPNNKTGQQIENIIRIKIEKQFKAINHPSKNNKGIP |
| | | KSLFADTNVRVNAIKLKKDLEAELDMLNKKHIAFKENQKASSNYDKLLK |
| | | EHQFTPKNKRPELRKYVFYKSEKGEEATWLANDIKRFMPKDFKTKWK |
| | | GCQHSELQRKLAFYDRHTKQDIKELLSGCEFDHSLLDINAYFQKDNFE |
| | | DFFSKYLENRIETLEGVLKKLHDFKNEPTPLKGVFKNCFKFLKRQNYVTE |
| | | SPEIIKKRILAKPTFLPRGVFDERPTMKKGKNPLKDKNEFAEWFVEYLE |
| | | NKDYQKFYNAEEYRMRDADFKKNAVIKKQKLKDFYTLQMVNYLLKEV |
| | | FGKDEMNLQLSELFQTRQERLKLQGIAKKQMNKETGDSSENTRNQTY |
| | | IWNKDVPVSFFNGKVTIDKVKLKNIGKYKRYERDERVKTFIGYEVDEK |
| | | WMMYLPHNWKDRYSVKPINVIDLQIQEYEEIRSHELLKEIQNLEQYIY |
| | | DHTTDKNILLQDGNPNFKMYVLNGLLIGIKQVNIPDFIVLKQNTNFDKI |
| | | DFTGIASCSELEKKTIILIAIRNKFAHNQLPNKMIYDLANEFLKIEKNETY |
| | | ANYYLKVLKKMISDLA |
| 25 | Cas13 | MEDKTTGAGISYDHTLMEDKHFFGGFLNLAQNNIDALLKAFKERFNV |
| | | RYQSKQFAEVCFSDKLPDQDYLDRTLFLETHLPFIKYIGGKEANNRGTF |
| | | RKNITLFFESIEQLRNFYTHYYHKPILFPEELYENLDRIFVEVSKEVKTHKV |
| | | KNDQTRHLLTKNLANELDIRYKKNVEKLKELKAQGKKVNIHDKEAIKNS |
| | | VLNNAFNHLIYKKEEDVFATEAYKSKYNLEDPSKNGISLSQSGLLFLLSM |
| | | FLNKKDIEALKSRVKGFKAKIIRDGEENISGLKFMATHWVFSSLSFKNV |
| | | KHKLSTDFHKETLLIQIVDELSKVPDEVYKTFDKQTQEEFIEDINEYMKV |
| | | GNKDLSLEESTVIHPVIRKRYDNKFNYFALRFLDEFAGFPTLRFQVHIGN |
| | | YIHDRRIKNIDGTAFQTERSVKERIKVFGKLSQMSNLKAEYVSGLMDEP |
| | | VDTGWEIFPNPSYNIIENNIPIYIEMGDHFNDEVLQSKMARKKQKPEE |
| | | LKDRNSAKASKESMIQTLQNDKGLMDVITVSPTAQLSLNELPAILYELL |
| | | VKKTPAKTIEKKLVGKLNQRLKEIKNYNPEKPLPASQISKRLRLNREEGSI |
| | | NTKKIIALLQKELNYTQEKLDLLEKNRKEYGKKVDGKILRKYVFGLKEIG |
| | | NLATDMAMDIKRFMPANVRKEWKGYQHSQLQQSLAFYDKRPEEAF |
| | | NILQEVWDINREKSLWDTWILNAFQTSGNFERFFELYHEGRKKYIQQ |
| | | QLENIDRYTDNKKFLQKFINQQFPTNFLEKRLYTLESLEIEKLKILSKPFIL |
| | | PRGTFDEKPTFIMGEKVTENPELFADWYTYGYQQHEFQKFYSWPRDY |
| | | KDLLQNEQKRDPDFAENKKGLSDLKQLELLQLKQD111KKIKTQDLYLKLI |
| | | MDALFIEVFGQEADISLNDLYLTQEERLEKEKLALKQHQRVEGDDSPN |
| | | VIKDNFIWSKTMPYKHDKIYEPQVRLKDFGKFKHFLLDDKVAKILSYDL |
| | | QETWNKNELEIQINTGQDSYEVIRREELLKEIQLLEKQILETFSHTLDEH |
| | | PKEFEDEKGNPNFKMYMANGVIRKGSSTTAKDEADWLEHEKDFDNL |
| | | SLEIFNSKSEITQLTFLIVLIRNKFGHNQLPIKQFYEIIQNEYSITGETISRLY |
| | | LNFIIYAKARLKDLM |
| 26 | Cas13 | MEEKLGKGVEYNPFKKEDKYYFGGYFNLAENNINEVFKEVKKRLGETN |
| | | SSSNIELLNNVFRKEMSLVDYEKWVNAFADYFPIVNYLDRETIKKGEKV |
| | | VEVPREKRIECFRDMFKGLINTISQLRHYYTHYHHEPIEIDDKILSFLDEV |
| | | LFNTIITTKNKYLKTDKTKELIKDSLQEELDILCKLKVKYLESKRKRFDRKD |
| | | KGAIENAVYNDVFRRFIYKDEKGNESLKDIIRTKQIKVHQNSSYLELPISS |
| | | SGIIFLLSLFLNKKEVESLKSNIRGYKGKSKSEETTPEKNGLLFMTTHRIYS |
| | | VLAYKGLKKRIKTSVKGDKETLLMQMIDEVSKVPHCIYQNLDQTLQAT |
| | | FIEDWNEYFKDNEENEENLENSRVLHPVIRKRYEDKFNYFAIRFLDEYA |
| | | EFPSLRFQVNLGNYVHHKATKKFGNSEVTTERVIKDKITVFGRLSEVNK |
| | | AKADFFKNETELDPAWELFPNPSYEFPKEKGNNDKDAGKIGIQVKLLN |
| | | KDIEAVLNESKNTLNNKTRKSDKISKKEIINKIVQINDDTKYNNKNIIYQ |
| | | GNAIAYLSLNDIHSLLYELLVIGTKGDKLERKVVEKIQQQVTEIRNKDTS |
| | | AKILSKYKDSEESNTIDKKKLVIDLKYEYDKLQDLLKEHKNREEDYIQTKK |
| | | KKKDSPKRKYILYHNEKGQVAVWLSNDIKRFMPQNFKEKWKGYQHS |
| | | EFQKSLAYYETNKEMLKIILQDLDLEQFPFDIKSCFYKNTLEDFYNRYLSL |
| | | RISYLENVIDRVECFSNEPKAFKSVLKECFVFLKKQNYTNHSLDEQVKKI |
| | | LANPIFIERGFLDTKPTMIQGVKFSENKGCFADWFVHYKEYEHYQKFY |
| | | DTNLYPVESIEDKERQKLEATIKKQQKNDVFTLLMIKKIFNDLFNQDFE |
| | | ANLYEMYQSKEEREKNQLVAKETQNRNLNFIWNKPIAIDLFDGKVKID |
| | | EVKLKDVGSFRKYENDKRVQTFITYIPEIQWIPYLPNTWEGINLPVNVIE |
| | | RQIDRYEKVRSEELLKEVQAIEKYIYEQVNDKTELLQNGNQNFKNYLVN |
| | | GLLKQIQGIDVSNFKFINQQKFETINVKDLDNEASALEQKVYVLINIRN |
| | | QFSHNQFPKSAFYQFCQKILSIEEDELFADYYLRLFKLLRNELLD |
| 27 | Cas13 | MNTRVTGMGVSYDHTKKEDKHFFGGFLNLAQDNITAVIKAFCIKFDK |
| | | NPMSSVQFAESCFTDKDSDTDFQNKVRYVRTHLPVIGYLNYGGDRNT |
| | | FRQKLSTLLKAVDSLRNFYTHYYHSPLALSTELFELLDTVFASVAVEVKQ |
| | | HKMKDDKTRQLLSKSLAEELDIRYKQQLERLKELKEQGKNIDLRDEAGI |
| | | RNGVLNAAFNHLIYKEGEIAKPTLSYSSFYYGADSAENGITISQSGLLFLL |
| | | SMFLGKKEIEDLKSRIRGFKAKIVRDGEENISGLKFMATHWIFSYLSFKG |
| | | MKQRLSTDFHEETLLIQIIDELSKVPDEVYHDFDTATREKFVEDINEYIR |
| | | EGNEDFSLGDSTIIHPVIRKRYENKFNYFAVRFLDEFIKFPSLRFQVHLG |
| | | NFVHDRRIKDIHGTGFQTERVVKDRIKVFGKLSEISSLKTEYIEKELDLDS |
| | | DTGWEIFPNPSYVFIDNNIPIYISTNKTFKNGSSEFIKLRRKEKPEEMKM |
| | | RGEDKKEKRDIASMIGNAGSLNSKTPLAMLSLNEMPALLYEILVKKTTP |
| | | EEIELHKEKLDSHFENIKNYDPEKPLPASQISKRLRNNTTDKGKKVINPE |
| | | KLIHLINKEIDATEAKFALLAKNRKELKEKFRGKPLRQTIFSNMELGREA |
| | | TWLADDIKRFMPDILRKNWKGYQHNQLQQSLAFFNSRPKEAFTILQD |
| | | GWDFADGSSFWNGWIINSFVKNRSFEYFYEAYFEGRKEYFSSLAENIK |
| | | QHTSNHRNLRRFIDQQMPKGLFENRHYLLENLETEKNKILSKPLVFPR |
| | | GLFDTKPTFIKGIKVDEQPELFAEWYQYGYSTEHVFQNFYGWERDYN |
| | | DLLESELEKDNDFSKNSIHYSRTSQLELIKLKQDLKIKKIKIQDLFLKLIAG |
| | | HIFENIFKYPASFSLDELYLTQEERLNKEQEALIQSQRKEGDHSDNIIKD |
| | | NFIGSKTVTYESKQISEPNVKLKDIGKFNRFLLDDKVKTLLSYNEDKVW |
| | | NKNDLDLELSIGENSYEVIRREKLFKKIQNFELQTLTDWPWNGTDHPE |
| | | EFGTTDNKGVNHPNFKMYVVNGILRKHTDWFKEGEDNWLENLNET |
| | | HFKNLSFQELETKSKSIQTAFLIIMIRNQFAHNQLPAVQFFEFIQKKYPEI |
| | | QGSTTSELYLNFINLAVVELLELLEK |
| 28 | Cas13 | METQILGNGISYDHTKTEDKHFFGGFLNTAQNNIDLLIKAYISKFESSPR |
| | | KLNSVQFPDVCFKKNDSDADFQHKLQFIRKHLPVIQYLKYGGNREVLK |
| | | EKFRLLLQAVDSLRNFYTHFYHKPIQLPNELLTLLDTIFGEIGNEVRQNK |
| | | MKDDKTRHLLKKNLSEELDFRYQEQLERLRKLKSEGKKVDLRDTEAIRN |
| | | GVLNAAFNHLIFKDAEDFKPTVSYSSYYYDSDTAENGISISQSGLLFLLS |
| | | MFLGRREMEDLKSRVRGFKARIIKHEEQHVSGLKFMATHWVFSEFCF |
| | | KGIKTRLNADYHEETLLIQLIDELSKVPDELYRSFDVATRERFIEDINEYIR |
| | | DGKEDKSLIESKIVHPVIRKRYESKFNYFAIRFLDEFVNFPTLRFQVHAG |
| | | NYVHDRRIKSIEGTGFKTERLVKDRIKVFGKLSTISSLKAEYLAKAVNITD |
| | | DTGWELLPHPSYVFIDNNIPIHLTVDPSFKNGVKEYQEKRKLQKPEEM |
| | | KNRQGGDKMHKPAISSKIGKSKDINPESPVALLSMNEIPALLYEILVKK |
| | | ASPEEVEAKIRQKLTAVFERIRDYDPKVPLPASQVSKRLRNNTDTLSYN |
| | | KEKLVELANKEVEQTERKLALITKNRRECREKVKGKFKRQKVFKNAELG |
| | | TEATWLANDIKRFMPEEQKKNWKGYQHSQLQQSLAFFESRPGEARS |
| | | LLQAGWDFSDGSSFWNGWVMNSFARDNTFDGFYESYLNGRMKYFL |
| | | RLADNIAQQSSTNKLISNFIKQQMPKGLFDRRLYMLEDLATEKNKILSK |
| | | PLIFPRGIFDDKPTFKKGVQVSEEPEAFADWYSYGYDVKHKFQEFYAW |
| | | DRDYEELLREELEKDTAFTKNSIHYSRESQIELLAKKQDLKVKKVRIQDL |
| | | YLKLMAEFLFENVFGHELALPLDQFYLTQEERLKQEQEAIVQSQRPKG |
| | | DDSPNIVKENFIWSKTIPFKSGRVFEPNVKLKDIGKFRNLLTDEKVDILL |
| | | SYNNTEIGKQVIENELIIGAGSYEFIRREQLFKEIQQMKRLSLRSVRGMG |
| | | VPIRLNLK |
| 29 | Cas13 | MENQTQKGKGIYYYYTKNEDKHYFGSFLNLANNNIEQIIEEFRIRLSLK |
| | | DEKNIKEIINNYFTDKKSYTDWERGINILKEYLPVIDYLDLAITDKEFEKID |
| | | LKQKETAKRKYFRTNFSLLIDTIIDLRNFYTHYFHKPISINPDVAKFLDKN |
| | | LLNVCLDIKKQKMKTDKTKQALKDGLDKELKKLIELKKAELKEKKIKTW |
| | | NITENVEGAVYNDAFNHMVYKNNAGVTILKDYHKSILPDDKIDSELKL |
| | | NFSISGLVFLLSMFLSKKEIEQFKSNLEGFKGKVIGENGEYEISKFNNSLK |
| | | YMATHWIFSYLTFKGLKQRVKNTFDKETLLMQMIDELNKVPHEVYQT |
| | | LSKEQQNEFLEDINEYVQDNEENKKSMENSIVVHPVIRKRYDDKFNYF |
| | | AIRFLDEFANFPTLKFFVTAGNFVHDKREKQIQGSMLTSDRMIKEKINV |
| | | FGKLTEIAKYKSDYFSNENTLETSEWELFPNPSYLLIQNNIPVHIDLIHNT |
| | | EEAKQCQIAIDRIKCTTNPAKKRNTRKSKEEIIKIIYQKNKNIKYGDPTAL |
| | | LSSNELPALIYELLVNKKSGKELENIIVEKIVNQYKTIAGFEKGQNLSNSLI |
| | | TKKLKKSEPNEDKINAEKIILAINRELEITENKLNIIKNNRAEFRTGAKRK |
| | | HIFYSKELGQEATWIAYDLKRFMPEASRKEWKGFHHSELQKFLAFYDR |
| | | NKNDAKALLNMFWNFDNDQLIGNDLNSAFREFHFDKFYEKYLIKRDEI |
| | | LEGFKSFISNFKDEPKLLKKGIKDIYRVFDKRYYIIKSTNAQKEQLLSKPIC |
| | | LPRGIFDNKPTYIEGVKVESNSALFADWYQYTYSDKHEFQSFYDMPRD |
| | | YKEQFEKFELNNIKSIQNKKNLNKSDKFIYFRYKQDLKIKQIKSQDLFIKL |
| | | MVDELFNVVFKNNIELNLKKLYQTSDERFKNQLIADVQKNREKGDTSD |
| | | NKMNENFIWNMTIPLSLCNGQIEEPKVKLKDIGKFRKLETDDKVIQLLE |
| | | YDKSKVWKKLEIEDELENMPNSYERIRREKLLKGIQEFEHFLLEKEKFDG |
| | | INHPKHFEQDLNPNFKTYVINGVLRKNSKLNYTEIDKLLDLEHISIKDIET |
| | | SAKEIHLAYFLIHVRNKFGHNQLPKLEAFELMKKYYKKNNEETYAEYFH |
| | | KVSSQIVNEFKNSLEKHS |
| 30 | Cas13 | MEKTQTGLGIYYDHTKLQDKYFFGGFFNLAQNNIDNVIKTFILKFFPER |
| | | KDKDVNAAQFLDICFKDNDADSDFLKKTKFLRMHFPVIGFLASNNDK |
| | | AGFKRKFSLLLKAISELRNFYTHYYHQPIEFPSELFELLDDIFVETTSEIKKL |
| | | KKKDDKTQQLLNKNLSEEYDIRYQQQIERLKELNAQGKKIPLNDETAIR |
| | | NGVFNAAFNHLIYKDGGDLKPSRVYQSSYSEPDPAENGTSLSQSSILFL |
| | | LSMFLERKETEDLKSRVKGFKAKFIKNGEEKISNLKLTATHWVFSYLCFK |
| | | GIKQKLSTEFHEETLLIQIIDELSKVPDEVYSAFGAKTKQKFVEDINEYMK |
| | | EGNADLSLEDSKVIHPVIRKRYENKFNYFAIRFLDEYLSSTSLKFQ.VHVG |
| | | NYVHDRRIKNINGTDFQTERVVKDSIKVFGRLSKISNLKADYIKEQLSLP |
| | | NDSNGWEIFPNPSYVFIDNNVPIHIQTDEATKNGIKLFKDTRRKEQPEE |
| | | LQKRKGKLSKHNIVEIIFKETKGKDKPRVDEPLALLSLNEIPALLYQILEKG |
| | | ATPEDIELIIKNKLAERFEKIKNYDPETPAPASQISKRLRNNTTAKGQETL |
| | | NAEKLSILIEREIEDTETKLDAIEEKRRKAKKEYRRNSPQKSIFSNSELGRI |
| | | AAWLADDIKRFMPAELRKNWKGYQHSQLQQSLAYFEKRPQEAFLLLK |
| | | EGWDTSDGSSYWNIWVINSFSETEDFEKFYENYLRKRAKYFSELAGNI |
| | | KQHTHNAKFLRKFIKQQMPADLFPKRHYILKDLETEKNKVLSKPLVFSR |
| | | GLFDSNPTFIKGVKVTENPELFAEXXNGIATGTKRNIPSSISMAGKETI |
| | | MSF |
| 31 | Cas13 | MEQNKLGKGIDYNPFKTVDKHYFGGFFNLADNNIQEVFDEINIRYKN |
| | | GNLKPKVAIERYTTENTSLVEYEKFVAILTEYFPIVKEIDQKNKKDSNDK |
| | | VIEKTRIERITDFRDAFILFIETIEKLRSYYTHYQHDDITIDNQLFIHLDKILL |
| | | NTVLETKKKYLKTDKTKELLKNSLQAELKELYHLKINQLEQKKNEVDALI |
| | | KEQKSKGKKTDKPFKYSKDRDQIINSIYNDAIRPFLYENANKVELSDKKK |
| | | TAFNEKDASASERDFNLPISSSGIIFLLSCFLNRKEIEDLKANIKGYKGKVI |
| | | KGETFDLEKNSIRFMATHRIYSVMCYKGLKNKIRTSESATKETLLMQMI |
| | | DELSKIPDIVYKNISTDLQNTFTEDWNEYYKDNIENNENLENSKVIHPVI |
| | | RKRYEDKFNYFAIRFLDEFVDFPSLRFQVHLGNYIKHSMPKNIGSVTTT |
| | | REIKNKIFVFGKLNEINQSKNDFFNKNKEEEQETNWEIFPNPNYHFPM |
| | | ENSDELKNANKIGIYIDLKDKRKKDTLNEAIKKREKETSIYKKDLVHQIID |
| | | KNLDMHIGQPVAYLSMNDIHAIIFSILSQNVFTKDNKLNGGDIEKKIKD |
| | | QINNQITEITEKDASIKILKNHSDNNSNYPNTHKLYDDISNEIEXXXXXX |
| | | XXXXXXXXXXXXXXXXXXXXXXXXXXXXXXXXXXXXXXXXXXXXXXXXX |
| | | XXXXXXXXXXXXXXXXXXXXXXXXXXXXXNEIEVLDKLMQKHEKRVKEY |
| | | INTQEDKKYKPARKHILYNSEKGEIATWLANDIKRFFPKEFKENWKGH |
| | | YHSEFQRNLAYYETNKKEVKTILNDLDYRKEIPFIDFSKNTLADFYFEYLK |
| | | KRKIYHKNLWVEVNKLIKGENINKEKLFDNCFRIYKRKNYVSNVIDEKV |
| | | NTILSNPIFIERGFIDEKPTIIPKMPLEGNEEHFAAWFVAFKSFKNNEFQ |
| | | NFYDTNKYPLETKDKTNSELKKIQTKTYNQKKNDWATWLIVQYIFKDIF |
| | | STDLQNVKLSELFQTREQRIQNQVKALDGERNQNFIWNRTIDLQLNE |
| | | KIKIPNVKLKDIGNFRKYVNDSRVEAFLRYNDITQWMAYLPSNWQKE |
| | | DESKPKPVNVIQLQLDDYEKIRREELLKEVQKLEKTIYNNTNVKTVLLQ |
| | | DGNPNFKNYVLNGLLEEIKGINISAFTVLHEKTNFDKIDFNVLENCSEIE |
| | | QSATLIILIRNKFAHNQLPSSDCYQFCSKILTRDTEQTYANYYLKLFMILK |
| | | DKL |
| 32 | Cas13 | MEETTTMGKGVAYDHTLFKDKHYFAGYLNLAVNNIENVFKTVYKNRF |
| | | DIKQHNLYKILDSLDGQISEPDYIERVSFLKQYFPVLHYLDLHPDNKRFT |
| | | KEEDKVKARRRYLINNLRLLIETLSKLRDFYTHYYHKPLSIEQNTFSLIDNI |
| | | FLNVVIDVKRQKKKNDHTRQLLKDSLKEEMDILYQKTKASLKEKQKEN |
| | | TRIKLDSETINNTIFNNSFSHLIYRRKKADNDILSASCKSEYKGEPTENGI |
| | | NVSVDGLLFFLGIFLSRKESNDLRGRIKGFKGTVIKDLPDFPNEKNNSLK |
| | | FMATHWVFTYLNIKPIKQKLNTNFSRETLLLQIVDELTKIPNEIYRNLCF |
| | | KKQQEFVEDINEYIKEGDDIDTLNSSTVIHPVIRKRYENKFNYFVLRYLD |
| | | EFVSFNSLRFQIYLGNYVHHIQRKKLSGTEYETERVIKEKINVFGKLSEVS |
| | | NIKGDYFIQNNPDNEALGWEIYPNPSYNFTGNNIPIYFDINDQDKEKIN |
| | | EYKSIRNFSEKRILRKKNKKNKQEIFDLINNTLTTRVFTAEPTAILSLNELP |
| | | ALLYTILCENKTASEIENLLRRTYLKRLNTIKNYQPGTLPQSKITKNLNKS |
| | | TNQESLDVSKLIKAMKHEISISNEKLTLIKKNQNEVKDTSHRRKYVFNSK |
| | | ELGIEATWLANDLKRFMPKKVRENWKGYMHSQLQNSIAYYSQKPKE |
| | | ALSILSSVWNFNDDNYIWNEGIKKAFNEKEFEKFYCKYLASRNKTLEKL |
| | | KENLDNLEYKTDKRKLDKFIKQQNLDCLFHIRTYTIDSTQEQINKLLAKP |
| | | LVFPRGIFDSKPTFVKNESVTEKPELFADWYTYTYKEHPLQEFYSFTKDY |
| | | ECNFKKEKLTVKEFVKNQEQLNPEEQLNLFKLKEDLSIKCIKNQDLFLKL |
| | | VVDNIYNKIFEYNIDISLKNLYISRKERIAIGLKAKELNQINDSYIWGKTIL |
| | | YQDKQIRETKVQLKDINKIKRFLEEDKVKQILSYDINKQWEIEELKYELYI |
| | | KPNSYEVIRREKLFKAIQEFESYILTINNFDGSNHPSILEYNSNPRFKHYV |
| | | VNGLLLKKGLATNEEIEWLLAKGQKEFNTFDKSIVEKPEIIQKAFLLVLIR |
| | | NKFAHSQLPIKEYYEMIRSYTKNIENLNTTEIIFQFTTNTINELKR |
| 33 | cas12b | MATRSFILKIEPNEEVKKGLWKTHEVLNHGIAYYMNILKLIRQEAIYEH |
| | | HEQDPKNPKKVSKAEIQAELWDFVLKMQKCNSFTHEVDKDVVFNILR |
| | | ELYEELVPSSVEKKGEANQLSNKFLYPLVDPNSQSGKGTASSGRKPRW |
| | | YNLKIAGDPSWEEEKKKWEEDKKKDPLAKILGKLAEYGLIPLFIPFTDSN |
| | | EPIVKEIKWMEKSRNQSVRRLDKDMFIQALERFLSWESWNLKVKEEY |
| | | EKVEKEHKTLEERIKEDIQAFKSLEQYEKERQEQLLRDTLNTNEYRLSKR |
| | | GLRGWREIIQKWLKMDENEPSEKYLEVFKDYQRKHPREAGDYSVYEF |
| | | LSKKENHFIWRNHPEYPYLYATFCEIDKKKKDAKQQATFTLADPINHPL |
| | | WVRFEERSGSNLNKYRILTEQLHTEKLKKKLTVQLDRLIYPTESGGWEE |
| | | KGKVDIVLLPSRQFYNQIFLDIEEKGKHAFTYKDESIKFPLKGTLGGARV |
| | | QFDRDHLRRYPHKVESGNVGRIYFNMTVNIEPTESPVSKSLKIHRDDF |
| | | PKFVNFKPKELTEWIKDSKGKKLKSGIESLEIGLRVMSIDLGQRQAAAA |
| | | SIFEVVDQKPDIEGKLFFPIKGTELYAVHRASFNIKLPGETLVKSREVLRK |
| | | AREDNLKLMNQKLNFLRNVLHFQQFEDITEREKRVTKWISRQENSDV |
| | | PLVYQDELIQIRELMYKPYKDWVAFLKQLHKRLEVEIGKEVKHWRKSL |
| | | SDGRKGLYGISLKNIDEIDRTRKFLLRWSLRPTEPGEVRRLEPGQRFAID |
| | | QLNHLNALKEDRLKKMANTIIMHALGYCYDVRKKKWQAKNPACQIIL |
| | | FEDLSNYNPYEERSRFENSKLMKWSRREIPRQVALQGEIYGLQVGEVG |
| | | AQFSSRFHAKTGSPGIRCSVVTKEKLQDNRFFKNLQREGRLTLDKIAVL |
| | | KEGDLYPDKGGEKFISLSKDRKLVTTHADINAAQNLQKRFWTRTHGFY |
| | | KVYCKAYQVDGQTVYIPESKDQKQKIIEEFGEGYFILKDGVYEWGNAG |
| | | KLKIKKGSSKQSSSELVDSDILKDSFDLASELKGEKLMLYRDPSGNVFPS |
| | | DKWMAAGVFFGKLERILISKLTNQYSISTIEDDSSKQSM |
| 34 | cas12b | MATAVDTSTTRAYTLRLSGGNNWRELLWQTHVAVNRGAWVWGD |
| | | WLLTLRGGLPASLADGDAERRVVLALSWLSVESPASLAPQAHIVAYGS |
| | | DARDERNRKVTERFRDILRRMGIKQQQEQEWLDACLPALMASIREDA |
| | | VWVDRSACFAEAQQCYRGLSSEWARKTLFDFLGGEDDYFKPSAKEG |
| | | ASSKAKDFVQKAGRWLSRHWGAGKKSDPRDISTRLGKLAGVDPKAID |
| | | GHTGRAALEDLLRTLGSRPAQNADAEKLYRQLKRAVGWKGRPSKGA |
| | | VALKKIRDAERVPNDLWKEIASTLREEAAVQSSQTSDHAAVPDWRSH |
| | | WPAEITGLPMPYRVDRDYIWEHGVMLDHALRRVSSAHTWIKRAEAE |
| | | RRRFQQDAAKMGSIPEEARNWLDAFRERRSSSSGATGDYLIRERAIN |
| | | GWDKVVQAWETLGPNSTRDQRIAAARDVQANLDEDEKFGDIQLFA |
| | | GFGDEHVDDPERCLADDRATCVWRNSSGRADGRILKDYVAATVAEH |
| | | NQRRFKVPAYRHPDPLRHPVFVDYGKSRWSINYSALTAAQQRRKTTQ |
| | | KLAQAKTDNTRAKLQQQLASTADLRSVTLGVWDGNRIVKISQRWRS |
| | | KRFWRDLDLDHFGSHPSAAVSRADRLGRVAARQDPGAAVYVAKVFE |
| | | QQDWNGRLQVPRRELNRLADVVYGKGADPDFGKLERLDPRARRLW |
| | | ERLSWFLTTSATVQPQGPWLDYVAAGLPSGIQYTKSRAGYYLNYDAN |
| | | HGRKGRARLCLARLPGLRVLSLDLGHRYAAACAVWQTLTIEQMTNEC |
| | | RQAAHPAPSNDDLFIHLRHPTHKPQKSGRKKGRPVTKTTIYRRIGPDK |
| | | LPDGTDHPAPWARLERQFLIKLQGEDRPARYASQKEIDEVNQFRNFV |
| | | GLEPIVDRPRVDDLHSDAVRVARLGLRRLADAARIAFAMTAAKKPISG |
| | | GHEVELTTAQRIEFLQDALLLWQSLAASRRYRDDWAEKLWQSWVVE |
| | | KLGGPQPAEIADDLPRSQRAASLKTARQSLRKVAEKLSDGQSPSAAEL |
| | | HRLWAERWQQRQTEWRRHLRWLRRLILPRRKDHQQEDRPLQRVG |
| | | GLSVKRIQTIRQLYQVLKAFRMRPEPSDLRKNIPAPGDRSLASFGRRIL |
| | | NHLERLREQRIKQLASRVVEAALGAGRISKPPGRDRRRPQQPVDRPC |
| | | HAVVIENLQHYKPEDSRLRRENRQLMDWQARNLRKYIVEGCELHGLL |
| | | FVEVSPAYTSRQDSRTGAPGLRCEDVSRTALQEAARRMHASHSRPSN |
| | | SSPGGSQTQFEREVCRWINEFKRVEGSSSSLSARQAVLKAFLHHQASI |
| | | PTSLSTILLPRRGGELFVSADPDSPLACGLQADLNAAANIGLKALTDPD |
| | | WMGAWWFVLVDRASGQPVEEQVQGCPIWLSCGPLSNSNPATIDPS |
| | | DSPTAARRSNGTGAKGRARANEYWWSSLSATTLPDHKAWQPTQDY |
| | | WRDIEQRVVKRLLRLLDGSEWSED |
| 35 | cas12b | MNRIYQGRVSKIEIKDSEGNFRNVPVGSPDTCPLWRHHRIFQDAVNY |
| | | YLVALGALAGTGSENAFVGLGSKDRVIHDLYSRLFDSWERFPRDMHG |
| | | ASSLRDSLRRTLPGLSERASLQDAFDAILSGNEANARERVLSLLSLIQDL |
| | | GGDIQKGSKRYFPFFCEPATKATFPRARVGLLKVEGKDFVPRLLWSSD |
| | | LEIAPDQVVEQLKFEYFANPNESVQPIEGNEARVRLIEALDNPQLGIEL |
| | | PIEILSDLRKRVHLIETDIRIPRYFFGGAGAELRKFRLDLFLIAAYVTPDPSI |
| | | LRALRNSFKEPSASKSSKKKDETEEVENLLRSLGDDPLILARGERGFVFP |
| | | SFTSLPTWVGANAQKPIWRDFDIAAFAEALKSLNQFTAKTEEREEKLK |
| | | KAEETLHYMLGISDAIPRSSDSETEEQAPSRPGKDPRWPLVAQLEKEL |
| | | GENLSEGTWQLSRSAMRGLRDIIGLWRKHPGASVVTLQKDVKTYQA |
| | | DEKHKREIGSVQLFLLLCEERYHALWQTETDDERGDESEENDDPARILS |
| | | DAIEVHQIRREVERFREPIRLTPAEPVFSRRLFMFSDLTDKLAKVKFGET |
| | | TEENSEVKSQFVEAAIALKEGENLKEARVRITFSAPRLHRDELLGGAESR |
| | | WLQPITAALGFSNPAPSVKFDSAVALMPDHMDDGRIRHLLNFPVNF |
| | | DSAWLHQSIGKADLWKSQFNGTKDKNLHLHWAGTARDTTRKNTW |
| | | WENRTIIENGFTVLSNDLGQRSAGAWALLKVTCSRPDTKHPVRSIGH |
| | | DGTREWFATVLATGIHRLPGEDQRILKNGKWATEQSGKKGRNATFSE |
| | | YEAACVLAKNLGCESVENWLGMSGEKSYPALNDQLVKIANRRITRLGT |
| | | YHRWSCFSPEKFEDPARRANVIGGQLAELSAYQDENVTVSADILKSGD |
| | | FEGFRHRAGAAFEALRTELEVHLVNLANLTAPLRQKVWSWQKRPDSS |
| | | GYGDLLMVDLDDCHPKIRGQRGLSMARLEQLEGLRRLFLRYNRSLDR |
| | | SPGIPAKFGREDVGRTSGEPCQALLVKIDRMKEQRVNQTAHLILAQAL |
| | | GVRLCPHRIEENERKSRDLHGEYEKIPGREPVDFIVIEDLSRYLSSQGRA |
| | | PSENSRLMKWAHRAVRDKLKMLAEEPFGIPVVETVPAYSSRFHALNG |
| | | QAGSRLHELHELEAYQQQSLINLAAKTDFQNRDRSKAAGELFEQFQA |
| | | LAKLNERRRAEGKKVPRTLYYPKSGGPLFLASRDGDTIHADVNAAINL |
| | | GLRAIAAPACIDIHRRLRATKEKEVYRPRVGNAREKSAFSKDDIIQPSGA |
| | | PSKKFASSSSPNFFYEPEDLKQANGEPLFDRAMFGEYSLVSGVSLWSM |
| | | VNNAIYIRCVELNRTRLHGKDPDDQIPM |
| 36 | cas12b | MSITRSIKVKLIVPRDASLEARQLREGLWATHLFVNDGCHYYERLLLEF |
| | | RQRDVCVGKDDAGKDVIVPAAEWADRLRARLGRNGMVPSHIEAALP |
| | | IFRELYENMVPSALKAKSGTGQAGRSWHSKLVSPTSRGGEASAARIDV |
| | | LRPLLPVSGDDPAFEPAARALIEEAGDELLTSTGRCPAWVTAYRKGPE |
| | | GSAWVEKLRIQLREAVEAGDFDPPSDPQILAAGAVPAAPPLGAGIDAL |
| | | RPLLPLLGGDPAFEPAARALVEDIGDELFTSTGRPPTWVTAHPTWVRA |
| | | HRKDAECLEAADDFKWVERLRQRLRDDAKAGKFEQPLHERLGALGAL |
| | | PVAKPIGAGRVVSRADLTVFERGAMELAIEHLIGWESAGHRARAQYV |
| | | ERKKRHDDLLQWIEAEAPDALLAVRAYEAARTIHLATLGELGAAPQYT |
| | | LRLREIRPWRKLREWLLQNPDATIDERRRRLATMQTNDPRGYGGEAL |
| | | AWLAAPERRALVEHPAGDVVTRIAVLNIRKSILDRSRLFPTCTLADPVE |
| | | HPRFAKFGKPGDKNSAGYALAVDGVRREAIIKILVPRQDGLLVPTDLR |
| | | VPFAPSGQMRDLRASGLDISYERQDGRGRQAAKLQGGNLMFDRTHF |
| | | ARCGAPGPEALGSVWIKVALDLSSPAASLAMKTATPVRTYLSTAVRGR |
| | | PESTKYEKAAPPEGFRVLSVHMGLRTAATASMLRFGAPEEGGHEVPV |
| | | SGLAGETLVAFHERTVTMKLPGEDPDTRTEANRGVAKRELRGLGRGI |
| | | GCLKAIRRASASATPEDRAEALVIIETHVGQGDRHGWAPAEAVGRLD |
| | | PHGDPDDWKTACAALYAAVEADLGVAISSWRKAARAGGATGMLGG |
| | | KSLWAVDHLERSFRFLRSWDLRARPHDGDPRRPRPGYASKLLHHIDG |
| | | VKDDRVKTTADRIVQAACGRAWIGGPTVKRGTQDVRLPGRWEQRG |
| | | PRADLILLPDLTHFRFRSDRPRAENSRLMRWAHRQLAIYVRMQAEVE |
| | | GILVADTGAAFTTRFDAWTGAPGVRCEPVTADHLRGIAKREDYWLAR |
| | | LLREGALKHLRIDPASLRVDDLVPMDHGKILVALDGVDLPGLRILDTDV |
| | | NASQGLGRRYIEGHGLAYRLPGARVPRGEGEREAAVVHIKGKRLASA |
| | | MGGTVVVLRASEGPGDITWTAEVYDRPQGARKALGLSLAAFNSIATA |
| | | AVDDEGPAPENDDEALEEEAEEALGIATGERIVFFRDPSGAVAGGGW |
| | | LEASAFWGIANRMVTDRLRELGRLG |
| 37 | cas12b | MSLNRIYQGRVAAVETGTALAKGNVEWMPAAGGDEVLWQHHELF |
| | | QAAINYYLVALLALADKNNPVLGPLISQMDNPQSPYHVWGSFRRQG |
| | | RQRTGLSQAVAPYITPGNNAPTLDEVFRSILAGNPTDRATLDAALMQL |
| | | LKACDGAGAIQQEGRSYWPKFCDPDSTANFAGDPAMLRREQHRLLL |
| | | PQVLHDPAITHDSPALGSFDTYSIATPDTRTPQLTGPKARARLEQAITL |
| | | WRVRLPESAADFDRLASSLKKIPDDDSRLNLQGYVGSSAKGEVQARLF |
| | | ALLLFRHLERSSFTLGLLRSATPPPKNAETPPPAGVPLPAASAADPVRIA |
| | | RGKRSFVFRAFTSLPCWHGGDNIHPTWKSFDIAAFKYALTVINQIEEKT |
| | | KERQKECAELETDFDYMHGRLAKIPVKYTTGEAEPPPILANDLRIPLLRE |
| | | LLQNIKVDTALTDGEAVSYGLQRRTIRGFRELRRIWRGHAPAGTVFSSE |
| | | LKEKLAGELRQFQTDNSTTIGSVQLFNELIQNPKYWPIWQAPDVETAR |
| | | QWADAGFADDPLAALVQEAELQEDIDALKAPVKLTPADPEYSRRQYD |
| | | FNAVSKFGAGSRSANRHEPGQTERGHNTFTTEIAARNAADGNRWRA |
| | | THVRIHYSAPRLLRDGLRRPDTDGNEALEAVPWLQPMMEALAPLPTL |
| | | PQDLTGMPVFLMPDVTLSGERRILLNLPVTLEPAALVEQLGNAGRWQ |
| | | NQFFGSREDPFALRWPADGAVKTAKGKTHIPWHQDRDHFTVLGVDL |
| | | GTRDAGALALLNVTAQKPAKPVHRIIGEADGRTWYASLADARMIRLP |
| | | GEDARLFVRGKLVQEPYGERGRNASLLEWEDARNIILRLGQNPDELLG |
| | | ADPRRHSYPEINDKLLVALRRAQARLARLQNRSWRLRDLAESDKALDE |
| | | IHAERAGEKPSPLPPLARDDAIKSTDEALLSQRDIIRRSFVQIANLILPLR |
| | | GRRWEWRPHVEVPDCHILAQSDPGTDDTKRLVAGQRGISHERIEQIE |
| | | ELRRRCQSLNRALRHKPGERPVLGRPAKGEEIADPCPALLEKINRLRDQ |
| | | RVDQTAHAILAAALGVRLRAPSKDRAERRHRDIHGEYERFRAPADFVV |
| | | IENLSRYLSSQDRARSENTRLMQWCHRQIVQKLRQLCETYGIPVLAVP |
| | | AAYSSRFSSRDGSAGFRAVHLTPDHRHRMPWSRILARLKAHEEDGKR |
| | | LEKTVLDEARAVRGLFDRLDRFNAGHVPGKPWRTLLAPLPGGPVFVP |
| | | LGDATPMQADLNAAINIALRGIAAPDRHDIHHRLRAENKKRILSLRLGT |
| | | QREKARWPGGAPAVTLSTPNNGASPEDSDALPERVSNLFVDIAGVAN |
| | | FERVTIEGVSQKFATGRGLWASVKQRAWNRVARLNETVTDNNRNEE |
| | | EDDIPM |
| 38 | cas12b | MPTRTINLKLQISPKTDEGRKIRSALWTTHSEINKAVAEIEKLLLLCRGE |
| | | KYYTTNSKDEEVEVPEPQVKTDALEMARAVQAKNGKAGTGSDEEVLS |
| | | ALRMLYEATVPSSVLDDKGKPLSGDAQSIGGSYAGPICDPETCRIKDV |
| | | DRLFESGPFAETASKKFTQLPAWFNEVTKKNFNKDEPEKFVKVGKDK |
| | | DEKFYEIDLRQADAWYESPEVKDIVSKNKAFNKDKWWKNKRDGVDT |
| | | WAAEFVKKQFDLRKDVRVSIREELWDRLGLLPLGSLYFKKPVGNKWN |
| | | RMAFRLAIAHLLSWESWNHQTLAEYTKYTKYKDGLIELAGASRSLEVR |
| | | FEPLRQYQKERHEELSRTSFVDDDRPFTIGARMIRAWGRVREAWRNK |
| | | GDGIDERRQILADLQ.TELKGKFGDPHLFLWLAEAGRESLWRDEDVLTT |
| | | FVEINIAQRDLERHRPYSLMTFADARLHPRWAMYEALGGTNLRNYEL |
| | | TPEGKVKIPLLICEKDKLSEKTFTIPLAPSGQLKSLEIKSLPKKKVKISYASA |
| | | HQFYAGIPGGSEILFDRLFMENRASSALANGSCGPAWLKLTVDVESKA |
| | | PPEWLDKKGRVQTPPTVHHFKTGLANKSKHTDKLEPSLRVLSVDLGLR |
| | | TFASCSVFELVDEKPAKGLFFETDHPHLWAKHERSFKLTLPGEEAGDD |
| | | PKVAQARREAMDEVYSLRRDMYRLKDILRLKIISAPNERREKLESKIAE |
| | | MREKQDARAVVTSNFFERLSEKCDLNPPMWEHSCNEIHRDAEKAFS |
| | | ARIGEWRKRTRKRPGSWEEWRETRSYHGGKSYWMIEYLEAVRKLLIG |
| | | WSTHGRDYGEINRQNKKRYGTVASKLLKHINKLKEDRTKAGTDLIIQA |
| | | ARGYIPLPGKGWMEKYRPCRVILFEDLARYRFKVDRPRRENSQLMKW |
| | | GHREIINEATLQGEIYGMVVETAGAGFSSRFHAKTGAPGVRCRYLKED |
| | | DFENGAPKEFLVRQMKNLMKGDRLEPGLLVPWDGGELFATVDNGK |
| | | PIVIHADINAAQNLQRRFWTRFADAYRVNAVEENDNWVVTDTGVRV |
| | | LGALEMAVHGEADRKPRTGFTLHGTLQSGAELKAEGKKTDIKDVEED |
| | | KDDSISSEIIELQDEKERKGRETFFRDPSGGILDPGKWYGSKRFWGRAK |
| | | GAVTEALLDNQGANNALEEKPGNDELPF |
| 39 | cas12b | MATRSFILKIEPNEEVKKGLWKTHEVLNHGIAYYMNILKLIRQEAIYEH |
| | | HEQDPKNPKKVSKAEIQAELWDFVLKMQKCNSFTHEVDKDEVFNILR |
| | | ELYEELVPSSVEKKGEANQLSNKFLYPLVDPNSQSGKGTASSGRKPRW |
| | | YNLKIAGDPSWEEEKKKWEEDKKKDPLAKILGKLAEYGLIPLFIPYTDSN |
| | | EPIVKEIKWMEKSRNQSVRRLDKDMFIQALERFLSWESWNLKVKEEY |
| | | EKVEKEYKTLEERIKEDIQALKALEQYEKERQEQLLRDTLNTNEYRLSKR |
| | | GLRGWREIIQKWLKMDENEPSEKYLEVFKDYQRKHPREAGDYSVYEF |
| | | LSKKENHFIWRNHPEYPYLYATFCEIDKKKKDAKQQATFTLADPINHPL |
| | | WVRFEERSGSNLNKYRILTEQLHTEKLKKKLTVQLDRLIYPTESGGWEE |
| | | KGKVDIVLLPSRQFYNQIFLDIEEKGKHAFTYKDESIKFPLKGTLGGARV |
| | | QFDRDHLRRYPHKVESGNVGRIYFNMTVNIEPTESPVSKSLKIHRDDF |
| | | PKVVNFKPKELTEWIKDSKGKKLKSGIESLEIGLRVMSIDLGQRQAAAA |
| | | SIFEVVDQKPDIEGKLFFPIKGTELYAVHRASFNIKLPGETLVKSREVLRK |
| | | AREDNLKLMNQKLNFLRNVLHFQQFEDITEREKRVTKWISRQENSDV |
| | | PLVYQDELIQIRELMYKPYKDWVAFLKQLHKRLEVEIGKEVKHWRKSL |
| | | SDGRKGLYGISLKNIDEIDRTRKFLLRWSLRPTEPGEVRRLEPGQRFAID |
| | | QLNHLNALKEDRLKKMANTIIMHALGYCYDVRKKKWQAKNPACQIIL |
| | | FEDLSNYNPYEERSRFENSKLMKWSRREIPRQVALQGEIYGLQVGEVG |
| | | AQFSSRFHAKTGSPGIRCSVVTKEKLQDNRFFKNLQREGRLTLDKIAVL |
| | | KEGDLYPDKGGEKFISLSKDRKCVTTHADINAAQNLQKRFWTRTHGFY |
| | | KVYCKAYQVDGQTVYIPESKDQKQKIIEEFGEGYFILKDGVYEWVNAG |
| | | KLKIKKGSSKQSSSELVDSDILKDSFDLASELKGEKLMLYRDPSGNVFPS |
| | | DKWMAAGVFFGKLERILISKLTNQYSISTIEDDSSKQSM |
| 40 | cas12b | MYRGFCTVTATSGGWQSTTFLAGAQMADTTTRAYTLKLQGDRLAL |
| | | WRNHVIFNNGVKAWGEWLLCLRGGLPASLADHRDSLDVSKGEISRTF |
| | | KERTAAITPATIRQELKFKAATEKKVREEVASRRKKVTETAVAKELLAAR |
| | | RSELRRILALSWLCPETPVQLVPQAAIVAAADDSDREQKVLDGFRQILK |
| | | RKGVSDVAGWVQDCDATLRATIRSDAVWVDRTACFCSMPRAVRPS |
| | | EVDAAKHLFRLFGSMSDYFATASASSGPAEPKDFANTCRDWVSSFW |
| | | GGGEKSNKASILAALSAIAQIKPTRVVGKRGPAALAVIAGVLEQKPVDD |
| | | SVEALARAIGWLSGRPSAARLAINAIAASPRVSQKLWDRLVLACEKDC |
| | | GRQKSKLAFEGSASTIASALEPRLAGLTGMPYASTGRELIGEYATMLAF |
| | | AMRRVSQIHTKAKQAEAERRSFAPEQARLALVPSAARKWLEDYVEAR |
| | | TAASGAVDGYQLRKRALGGWADVVAAWSRCETSEDRIAAVRELQAD |
| | | WEKAGDVQLFEALAADDAICVWQSANGKTAASILTDYVRAAVADQN |
| | | ATRFKVPAYRHPDPLRSPTFVGFGNSQWSIAYSAQGEARERRKLLDRA |
| | | SGSAKDAERAREGLAREAVLQNVSLDLWAGDKMVPTQFRWQSRRLL |
| | | SDLALHSVPAMKGAKVTRATRFGRARIAAGPVLLDGIADDTPWNGRL |
| | | QAPRRQLEDLARILDAKGLPFDDESKWPPKVRSRLKHLGWFLTHSAKL |
| | | TPSGPWLDYVAGGLANGWKWAEGREGACLFREDNKDRKGRAKLILS |
| | | RLPGLRLLSVDLGLRTSAAAAVWQVVSKRQLTAAKDGAKSVSDTDLF |
| | | CLVRTGDRTQVYRRIGLSAWARLERQFLIRLDGEKAAARPATTNEWES |
| | | LQSFRAWLGCGIERRPEKLPPVDSLQQSAERLCRLGLRRLSDLARVAYL |
| | | LTAKERPIMGGRTAPLDEEGTVQAAQDALSILHALGSSEDFSDARLQG |
| | | IWRTAIGDTPPLAARLTKKQRQELREALRPAAEKLRGKAALGKELADL |
| | | WKERSAAWAKHLRWLRDWVIPRFDKRKNGERVRSARGVGGLSLDRI |
| | | ATIRGVYQIMRAYASRAEPTNLRAGVERLEKAAAKKLRPEFGRRMLAK |
| | | MERLRENRVKQIASRIVEAALGVGSEDRLHWERGRRRPTAAISDPRFA |
| | | PCHAVVIENLENYRPDEKRTRRENRGLMSWAARAVGKYLAEGCQLH |
| | | GLYLRQVSPAYTSRQDSRTGCPGLRCNDVRAQELLNPEGWIGRLVAR |
| | | AAEAVKEGKATPRQRLLVTLAESARAGIAESAAVRIIAPGGQLFIAADP |
| | | QSPASNGIHADMNAAANIGLVALLDPDWPAAWWRLPCKAATGYVD |
| | | ESKVGGSEAVPLGRAILEVGAEAGKVYVNAWSDPQDSAVSRREWTD |
| | | TKRYWRDVEERVVEILLASNRGGRRGKPGAVPF |
| 41 | cas12b | MATKSFEAKIVCKPDEKYTAEQKKQFLWFTHQVFNDGVRKVIPYVFK |
| | | MKRGELGPEFQAIYYAITSSQDAIGKLEAVINPDWTSGKIGKSDPNKW |
| | | KELLKYQELEKGFRQRLKEEGIKSTKKFRKELEDEKKKLAKEIGQKDIWA |
| | | DAAAILRNKNLLLFNRDELLPNLPSEFRRKIYEMTIQLIHGHQELVANW |
| | | EDEHAEWLIEKDKWEEEHPEYMNVRPIFEKFEKEQGKVKGSRIRWLA |
| | | YLDFLSSKPELANWRGKAKETIPLTKEERAGFRKPGQHFAAFFNKNPEL |
| | | QELDRLHKEYQEKFARTQSKRTPHPDGFKHRPTFTLPDAMRHPVWYS |
| | | FKGATDPTKGSTYRNLDLENCTLDLKVLTAMEGEGRNPGGMIQYAFE |
| | | PDERIKGFRYVGTTEKGKRAKGYIYYDPILEKERPAKIQGIKLVFRPPRP |
| | | DGTAYLIFSCQIEDEKPKIKIWKDKEEESPGEITKRKKTEVYPPELITLAID |
| | | FGQRHLGAITICKNNNGRPEPIRFIPAYPKRRKDRESKPVSAWLAKIPG |
| | | LTFNAVGMHEKEISAGMSRRFQDPKSIRQAGEKEGRKSKGQHIPETET |
| | | PWAHLREHIANMKEDHYKKAANLIIRTALQNGAQVILIENLRNYRPML |
| | | ERTNLENRRRMQWAVRQTAKFLEDTARPLGLIVRQVSSAYTSRFCSSC |
| | | GHPGARVSLPGQKNWEKFYAEKYGKERKMIAVAGGQFFCCPACKKII |
| | | NADINASLNMHKVFYQNFIWPGKIDKKDTKNFIWQGKNYNWDQIA |
| | | DDVQSFLDQKAGIKKEDDIPY |
| 42 | cas12b | MAYGSEAPDERNRKVTERFRIILSRMGINQQQEQEWLDACRPALTAS |
| | | IREDAVWIDRSACFAEAQQHYPGLSSEWARETLFDFLGGENDYFALP |
| | | DPEAAPSSEAKDFVQKAGGWLSRHWGAGKKSDSTAISTNLNRLAGV |
| | | ESKAIVGRCGCDALAVLLTTLGGWPAKNADSGTLYHQLKQAVGWKG |
| | | RPSRAAKALEKVRDAPEVTDALWRQTADTLRQEAVAQSSRAAGGSG |
| | | VPAWMPAWREDMEARLGMPYRGARDYIWEHSVMLDHALRRVSSA |
| | | HTWIKRAEAKRRRFQQDADKIGSIPAKAREWLDAFRERRFSASGALR |
| | | GYLIRERAIDGWDRVVQAWASLGPNCTREQRIAAARDVQANLDEDE |
| | | KFGDIQLFAGVGDEDEGDPQPCLADDDAICVWRDLNGRADSNILKDY |
| | | VAATVAKHDQQRFKVPAYRHPDPLRHPVYVDYGNSRWSIEYSALKAA |
| | | HQRRKTTEKLVQAKTDRARAKFQQKPADTPDLRGVTLGVWTGSSIEK |
| | | VSLHWHGKRFWKDLDLDHFGRDPSATVSRADRLGRVAASQHPEAA |
| | | VHVAKVFEQQDWNGRLQVPRHELQRLADLVYGKGGDPDFAKLGSL |
| | | DERRTRRQWEHLSWFLTTSTTIQPRGPWLDYVAQGLPQGIQYKKGR |
| | | NGYYLEYAANQGRKRRARLCLARLPGLRVLSLDLGDRYAAACAVWET |
| | | LTREQITQECHQAGHPGPSQDDLFIHLRHRTGKPQKSGRNKGKPVTK |
| | | TTIYRRIGPDLLPDGTPHPAPWARLQRQFLIRLQGEDRPARFASQHEID |
| | | GSNRFREFLGLPPLADRPRVDDLHRDMVRLARLGLRRLADAARIAFA |
| | | MTATKKPISGGREETLATEQRIEFLQDALVRWQALAASSRYRDDWAR |
| | | QAWQEWIVEKLGGPQPAEIADELPRSQQATRVETARRSLREVAAKLS |
| | | NPQSSSATELHGLWAARWQERQTKWRQYLRWLRRLILPRRKDYQQ |
| | | ANRQVHRVGGLSVKRLQTIRQLYQVLKAFRMRPEPSDLRKNIPAPGD |
| | | PSLASFGRRILHHRERLRQQRIKQLASRLVEAALGAGRISKRLGRDRRR |
| | | PRQSVDAPCHAVVIENLERYKPEDSRLRRENRQLMNWQARNLRKYIV |
| | | EGCELHGLLFVEVWPAYTSRQDTRTGAPGVRCEDVPRSVLEEATRRIR |
| | | ALGSAPSGSSRGRSETRFEREVCRWIHEFNRVVGSSSGLSPRQSVLKAF |
| | | LDHQAAIPTWRSTVRLPRRGGELFVSADANSPLANGLQADLNAAANI |
| | | GLKALTDPDWMGAWWFVLVKRDSGQPVPQQVQGSPIWESCTRLSS |
| | | PATVDSSDSPAGARRSKGRGARGRARATEYRWSPLSAMTMPDNKT |
| | | WWPTRDYWPEIERQIADRLLREQIDPENRF |
| 43 | cas12c | MKKTSPLKRSALRTARRQIARGCLPIGNRDISTTRTRVLPLADSVADAV |
| | | WNQARTAALTLRGFGSGSLFDLLLDLHASGLRLFSSNGEREGFLLKQK |
| | | FDAGKFDRAAAKDVGEDMPKFTAANLRAALVAIPRGGGPDTDAKAL |
| | | ATRLARAVGVKATKLDKPPKLLKDMAKELAMAFPTWKELSTANGEV |
| | | GAVIDDVARMYGLRWPSLRRGWAFRLPEVTRELGSPTLAFDPDAPVI |
| | | DETSATARFAAIVARYLPECGGLTDSAAAKGVQARITTTNANGLSWLF |
| | | GVGLRGMRDLPVDTVADTLAIDVTRGRDALRALVNDIKALPRLGEFG |
| | | DRVYVESRATLQGAVDSLIANYVGRLADLVASADALEHDQPRPPVLD |
| | | DADWKPAIFDGMGFTPWEVEDMLDARPVEVARLRLALGVLAGTTPA |
| | | VAGDFARALADVEAFGAWAARTEAVAALINARVKVLKAPESLRLRGV |
| | | LGGGRWKAVVSIHPDEGEPAQVIPQLDTQLQALLDDGQRAFDVLVA |
| | | DYTPTFAAALEHARSDMRASLADKGREAPSAESIDLLARRKLLDMVAR |
| | | VTRRGSPSLGHAFLAACAVQGLTRPGTATERSLRGHILSGEQALFVHP |
| | | YARARSIVRLEHAGLLRLDLDALLTAMERDAEQRADVREQIVLRFTRQ |
| | | SLLLGGLPGRIRLAKVPWTQEAAAASGVRGAPWLKLHPDDAGTVARS |
| | | EVIKAFTARFHLSANGLLYRLNRMRFLERYDIRCFIGDTLLFAPKAGAW |
| | | TPPEQYRHGKYAHWLSHPDLPRTEGGAVDVVPAARWLTEASRRADE |
| | | DGRASAVALLAQFPHEWVAACEFEGAPVYEGVFPCEGKIGGWMKRR |
| | | GYRLAPPRHFAGELLAAFKDASVSPHGLTFEREMLREGTTVRELSRRV |
| | | VAAYPIAVPTHPDAERPWSPLHLMGLDLGEAGLGVCLRHIGTGAETTL |
| | | LLPVRKTRLLAHREEHYRRKVQPRQAFRKGYGDAMELAVKAAIGEVC |
| | | GIIDNLIVRYRAVPVFESAVAQARGSNKMIQRVFAGVVQHYTFVANN |
| | | GAAQTVRQSHWFGAGRWSYTYGADLLPAARQMTEKQLLKAKAEAV |
| | | FRPAMGFPGVMASGYRTSLVCACCGEDVLDAVDAAAEGGQVALTT |
| | | DAEGSGVLDLGGRSLRIKLEAPSPNPIVQKAARRKRRRTPWEALADRT |
| | | WTLTHKTDRADLVATLRRGLRRPPASVQGHATSGWEFHCAACGHIA |
| | | QADVNAATNLVRRYDDRVRKMEQARAHWDDPSVRAKLASELAERA |
| | | AARS |
| 44 | cas12c | MLTTKFKLELPAGCPLREDAATFDECRKLYDVVEGCGNGTLTGFLFSVI |
| | | LSGFRIFPDGKTAEIFANRSVYDEDEFRSALVEAVGAPLPRFTVKALIKR |
| | | LQMEVRARGNKDNRFVAEVMMKEYRQTLCGKTLPKGVDESYVDRLF |
| | | EEMARELTSRYRSWNELKGDLLGACKAVDAALRGFGDFPSLATMVTR |
| | | AAARRLPKDSTIVFDPQSPCIDVQTIGVDAMPYAAVSTILSYPESVGEK |
| | | RRDFVQNHLTTPSAAGLSWLFNRGLELFSEESVEELCRLFHVPEDQRT |
| | | RIVQIQNAARATPRQSFFLKKGGAPLGYHDFRSAFAGRINSWTANYL |
| | | NRLEELQGLLHDLTDELRLPDLVRNGEDFLATTDCRREEVEILCRSFSRE |
| | | RDRAQTAVEHLIGADPLQVVSDVAAIEEYSRIVNRLCAIKEQIVNSLRQ |
| | | AEDDKASRWTALWSEVKDEFQPWEKLIRLPKLNGMSGGVPPAQDEL |
| | | ETILARYSDVGRGASEHFDAVMEWAAKTGAEGDVLKKFAETEQQRA |
| | | DQRAPGKYDGRELALRLVLQRVARVVRDRSDACAENVRQWFLKENV |
| | | FAERKDFNKFFFNRLGNLYVSPFSNRRHAGYKLSDGLVERSGAVWREL |
| | | LALVKEMRGAYASFSEAGETFLRLESLLMGMRIGALTKNIPAEVAALRL |
| | | DDETALESVSEGLKLQLQQAEVPPSVLAKAFNVYVSLLSGCLIALRRERF |
| | | FLRTKFSFVGNTALVYVPKEKSWPMPSRYEASPSWTPIFENDVLVRLS |
| | | TGEVEVAETFRRAVALWGRTTDPVLKKALRELFHQLPHDWCCQVSVR |
| | | SSGDMTPAKRKEDDRDVLIVEKKGKYDSTIISKKIAATALVRLVGPSTH |
| | | KERLNRLLLDVGEVACDMTLLADQEILQKVEDDRVHLSPGKLQFSLSV |
| | | PISTPAEQCEDEVKSERKSTHFRRIVAIDQGERGFAFAVFRLEDAGKKG |
| | | AQPIAQGFVNIPSIRRLIARVHSYRKGKQSVQKFSQRFDSTMFTLRENV |
| | | AGDVCGAIAGLMSRYRAFPVLERQVSNLASGGKQLELVYKMVNARFL |
| | | DDRIPMHSLERTSWWCGTSDWVIPDLWVEVPESYAVKAKKDEILEKD |
| | | GKFYRTLRITPGAGVNAKWTSRICSQCGGNAMELIEKAREEKVKTVTL |
| | | DANGEVTLFGRTLRLYKRPSEERSREARRRNERAPWTEPRADVRLSLD |
| | | DFRRAVAENMRRQPKSLQSRDTSQSRYFCVFTDCRCHNKEQHADIN |
| | | AAVNIGRRFLESLLRE |
| 45 | cas12c | MRRQHHGGQNARDWRRKVAAAALRQKESVFTYKFGLSVNDGDFDF |
| | | DAAARTYDITEGIERGSLIGLVCAVHLSGFRLFSKVAETRQFLNRSRYPE |
| | | NEFAQALAAHTEIENPSVTVQSIESVFVTPPRKQDGVARLWSADELAK |
| | | RLFQTWNNRSPREGERNHPELLLAQGIARAVTKAFSGWKELADNAV |
| | | HALTCADNYLATLGNRFPKLSDLPPLTAGSTQTGTLAFDPESPFLNMT |
| | | GNEDIWLHQVVAVCAGRLKRYMPEIDPSSRKFASRLTDSIVSSQNNGL |
| | | SWLFGNGLRFLRQSSIAQIAETLSVSQNEHRRVEQLKEFADAIPVNPFF |
| | | ATDGYAEFRGSVGGKISSWVSNYWKRICELTVLHSQPPDITIPEGLLAS |
| | | ENATLFSGQHTAAAGLVALSARLPSQVRDAGKALFVLSGDGVPRADD |
| | | IATVEDVAGELAELTGQLAMLDNRIQQEIERAQDANDEGRVGSLASL |
| | | RPNPTKELKEPPKLNRISGGTADAAGELARLETSLNDLIRARREHFYRLA |
| | | EWTGNTASLDPLPALAERERKALTDRGMDPTLAEADEYALRRLLHRIA |
| | | GMARRLSPNEAKRVRETMTPLFLKKREANLYFHNRAGALYRHPFSNS |
| | | RHQPYSIDLNRARATDWLAWLEERAREMLGLLGSGAPANHEYLRDLL |
| | | SIETFVFTTRLSGLPAQVPGYLAKPKSDLTNIPPLLAAQLDVDEVSRDVA |
| | | LRAFNLFNSAINGLSFRAFRDSFIVRTKFLRLGHDELFYVPKARAWKPP |
| | | ADYRSAKGKISKGLALPAVKRNEAGSILPRETTQGLSRAKFPEGSHALLS |
| | | QAPHDWFVELDLRHDKMPQLAGLPVKMNADGLKGWRARRRPTFR |
| | | LAGPPSFKTWLDRALTSTAVKLGDYTLILDQSFKQSLRVEDGEVRLSAE |
| | | PAGIKAEIAVPVIDARPFPETEAEALFDNIIGIDLGERRIGYAVFSLPALLK |
| | | SGNPTRVKPTVVGSVAIPAFRRLMAAVRRHRGSRQPNQKVSQTYSTA |
| | | LQQFRENVVGDVCNRIDTLCERYRAFPVLESSVANFETGANQLKLIYG |
| | | TVLRRYTFSNVDAHKSARSAYWYSANRWQHPYLFVREWNKAQRTFT |
| | | GSAKPLAIYPGVTIHPAGTSQICHRCGRNALRALRNMPDRTIRVGKDG |
| | | LIVLADSTIRLLERADYSDRELKTFKRRKQRPPLNMPVPEGARPRDQLE |
| | | RVLRRNMRQQPQSEMSPDTTQARFTCVYTDCGFEGHADENAAVNI |
| | | GRRFLERIDIEASSRT |
| 46 | cas12c | MTHAKKIPFPVLKRSTLRKARQRIAAGSITAGERPFNSTVTRVVPVKDP |
| | | VSDQVWAVAREAAMTLRGFGQGSLFDMLIHLHADGFRLFPSGRERE |
| | | AFFLKDLFDPTEFDDGARRAFGDVMPGFTANSLREILGAPARKCGKVT |
| | | SVEILLPRLSKGLGVKKSAAPPEVLSSLAAALCEAFPTWSLLTAVDGGV |
| | | GKVIDDVLRTHGSRLPSLEKAWSTNLPEVPKGLGVPTLAFDDQAPAQS |
| | | EQTPTGRFAGVVARYLAETFASNPEATAGDASKAVQAKVTTPNGNAL |
| | | SWLFAVGRRAMCSTTLDELAIGLNITSPRGRHALSSLKERMMALPALS |
| | | VLGERAYPDSRATLQGTVDSLIANYVNRLFELSSSATSIAQTKLILPAAIQ |
| | | GDTAVFDGMPFSAEDVGALFEQLPSEIAKLEHAVKVLVGKERTSTLGY |
| | | QKAVDDVDEFGVWASSVDAVIGQINARLKTLERAQEPLGKLMGDGK |
| | | LKRLVNIHEPEGPAVEIIPVLDQELQDVLTSCRTAFADLEARYPMTVAK |
| | | AQRHAEAEVRNALELASRKEGGLSLASADVPALAKRKILEPIISIARRSS |
| | | PAMATAVLTECLRQKLIVKGTGSERSLRGYVLSGEQVIYAHPLSRRRSI |
| | | VRLDREGLQNFDALEFLDALQKDATQRTNVRESLIVEMARQSLLLSAL |
| | | PDRIEIGAISWQTPSQNQHAPWANLRPVNGTVGRSETIKSFTAVFHS |
| | | RISGLLYRLNRQKFMEKYDLRCFIGSTLLFSPKNADWAPPPQYRHGRF |
| | | SALLARSDFPWEGAEGTHANAVRLAKFLIDETRNATDLQQAIAAKALL |
| | | AQLPHDWVVCCDFDGAPSYEGAFVSAGEVSAWAKRSGYLLTPPRHF |
| | | AGAFLEGFKSTKISPHGLTFERMLERDGDSVIETGRRVTAAFPITQEVA |
| | | PAAQPWKPRHLAGLDLGEAGLGVCLKNLDNGHEQTLLLKTRKTRLLA |
| | | HSAEHYRRKDQPRQVFRKQYNQSSENAIKAAIGEVCGLIDNLIARYDA |
| | | VPVFESQAAAARGSNRMVARVYAGVLQRYTYVVGNGAADATRTSH |
| | | WLGANRWSYSFGADVIPKVRDLSPEVLRSIKKPENVFRDALGFPGVLA |
| | | NAWRTSMICSVCGTDPIGALEEAIAANQISFVTDNEGEGSLDLGDGRK |
| | | VTLRVEVPTSSALTKREASRRKRRAPWEAKVGTVWTLTRKSHRDDLLT |
| | | TIRRSLRRPSSTFQGSTTKQWEFHCPCCGQIQQADVNAASNLVRRYF |
| | | VRASDNARARQHWADDSKRLAFIASMGPDRSAREEKVS |
| 47 | cas12d | MRKKLFKGYILHNKRLVYTGKAAIRSIKYPLVAPNKTALNNLSEKIIYDYE |
| | | HLFGPLNVASYARNSNRYSLVDFWIDSLRAGVIWQSKSTSLIDLISKLE |
| | | GSKSPSEKIFEQIDFELKNKLDKEQFKDIILLNTGIRSSSNVRSLRGRFLKC |
| | | FKEEFRDTEEVIACVDKWSKDLIVEGKSILVSKQFLYWEEEFGIKIFPHFK |
| | | DNHDLPKLTFFVEPSLEFSPHLPLANCLERLKKFDISRESLLGLDNNFSA |
| | | FSNYFNELFNLLSRGEIKKIVTAVLAVSKSWENEPELEKRLHFLSEKAKLL |
| | | GYPKLTSSWADYRMIIGGKIKSWHSNYTEQLIKVREDLKKHQIALDKL |
| | | QEDLKKWDSSLREQIEAQREALLPLLDTMLKEKDFSDDLELYRFILSDF |
| | | KSLLNGSYQRYIQTEEERKEDRDVTKKYKDLYSNLRNIPRFFGESKKEQF |
| | | NKFINKSLPTIDVGLKILEDIRNALETVSVRKPPSITEEYVTKQLEKLSRKY |
| | | KINAFNSNRFKQITEQVLRKYNNGELPKISEVFYRYPRESHVAIRILPVKI |
| | | SNPRKDISYLLDKYQISPDWKNSNPGEVVDLIEIYKLTLGWLLSCNKDFS |
| | | MDFSSYDLKLFPEAASLIKNFGSCLSGYYLSKMIFNCITSEIKGMITLYTR |
| | | DKFVVRYVTQMIGSNQKFPLLCLVGEKQTKNFSRNWGVLIEEKGDLG |
| | | EEKNQEKCLIFKDKTDFAKAKEVEIFKNNIWRIRTSKYQIQFLNRLFKKT |
| | | KEWDLMNLVLSEPSLVLEEEWGVSWDKDKLLPLLKKEKSCEERLYYSL |
| | | PLNLVPATDYKEQSAEIEQRNTYLGLDVGEFGVAYAVVRIVRDRIELLS |
| | | WGFLKDPALRKIRERVQDMKKKQVMAVFSSSSTAVARVREMAIHSL |
| | | RNQIHSIALAYKAKIIYEISISNFETGGNRMAKIYRSIKVSDVYRESGADT |
| | | LVSEMIWGKKNKQMGNHISSYATSYTCCNCARTPFELVIDNDKEYEK |
| | | GGDEFIFNVGDEKKVRGFLQKSLLGKTIKGKEVLKSIKEYARPPIREVLLE |
| | | GEDVEQLLKRRGNSYIYRCPFCGYKTDADIQAALNIACRGYISDNAKD |
| | | AVKEGERKLDYILEVRKLWEKNGAVLRSAKFL |
| 48 | cas13a | MPIVKKFGRSQ.TSLSDRKIVLKMETAARNIPDFLLSDPEAVIGQWASA |
| | | MDKIAKKPKGKDKPSSYQRKFRERLGKAIWADLTGPEGPLRDVPAAEL |
| | | EDLRKRWDRRVHPYPDGTKDGPKPATPKGRLYTRFAGEVGYGKADA |
| | | VAIARDIRIHLLETEFKTGGGTRDAGRAVRRASSIEKNVLKKARVPQRP |
| | | KPPQEAAWSKEDEDRYFIPHDVARKIVLAAKAQEKEDHRVAWRTAA |
| | | AVLFEHFGRIFQQDGRALSFAEAEKQMPGLLALHRAVEGYYRQALKR |
| | | HRKDRREHEARPGREKGTGRRKVSAILPKDKTALLALIGHQHQNREIA |
| | | ALIRLGRILHYEAGRRGNSDMVANINRNWPADVSESHYWTSAGQIEI |
| | | KRNEAFVRIWRSALSHANRTLGDWLSPDEVANDITMSWESKHEKSG |
| | | KRKTGKLEENREEAEAHAPVIFGGSAERLGTGDDFQKTLEAICEVFSQL |
| | | RHSSFHFRGLDGFKDALTKTVKTCDPGAVARLQDLHAEDQANREARL |
| | | KEDLRGAHAELFLDEGRLAEIWALLHPKSTEKTLPPLPRYSRVVTRAEN |
| | | TCNGLKLPKSVNRESMKVPAIHCRYILTRLLYQSGFRTWIAEAPAAQLN |
| | | RWIETATERAQKATVGITKNEADRARMVGQIKVPEGQGIRRFLDDLA |
| | | GLTATEFRVQAGYESDREAARDQAAFLENLNCDVMALAFDKYLSDHK |
| | | LGWLAGIDAESRPSETPLSNVDELPSSGSLGTPERWEAALYAVCHLIPV |
| | | SEVGRLLHQLRRWSNGQKATPDGGRLERLFELYLDMHDAKFDGSTPL |
| | | RDHDDLAVIFETTGIRDRVLPSSLQHGEHERLPLRGLREMLRFGNLRVL |
| | | APIFATAKVDQAMIGELEGLEARIGDAPSQVDRAQALRTEMHAALCK |
| | | KRKLAHDDKKSVKDYLTSLQTVIRHRRLANHVRLTNHVRTNEILMSV |
| | | MGRLADFSGIWERDLYFVTNALLYQAGLTPCDVFSKEPPKENRRSPLQ |
| | | EFENGQIVFALRKMQAQCDTHAGLVDQIKGETARLFHIAEGAPGNDP |
| | | RIQNRNWFAHFNALKPKTGDRLDLTADMNRARDLMAYDRKLKNAV |
| | | VSAIVTLLERENIVIAWTMKDHQLTDAVLAAKSIEHLKQNKIRENLRDE |
| | | RSLGYVAALFGGRVAEEAPDIMHDRTVFHLVGALTEEMEPAE |
| 49 | cas13a | MRIVRPYGESRTDLGGERGQTRVLVDNTAARARHEIPDFAQSHDALV |
| | | IAQWISVLDRIATKPQGTQGATRAQHAFRDRLGRAAWAQMCAADRI |
| | | SAAAQADPYVAALWRFKTHPYGDAKYRPRKGKDGKPLGEPKPQGR |
| | | WYGRFAANAEPEQADVAAIAALMDHHLHVAELRIDPKRPEKRKGLIE |
| | | ARAKSIEGNVLVAEPRKRPVGSWSREAITRYFMRQDVAAEIFAAARD |
| | | REQGLNDVPRGPVRLALAAKILHGHWTRLFHAPGTRTAYSIREAEEKE |
| | | PELFALHMAVKDAYAKLLKRRTQPKTLKKGVKPPQQAPVTTVLPKNA |
| | | GELLRLVQHRSRNRDLSALIRRGKLIHYTAFDIAAAAAEAESKTPDVPD |
| | | ADRLAYVLTHWPDDLSASRYLTSDGQSAIKRSEAFVRVWRHTIAMAS |
| | | LTLRDWASMNNDLGDVLGSANKVDQAIGRANFDPAWHDKKVRLLF |
| | | GARAALFPSDDDGRKALLASVIRAGLALRNSSFHFTGRGGFLAALKKL |
| | | GSEEVMVPSILAAAHALWREDATARAGRLRAALTGAHAAHYFEEDQ |
| | | NASILTLLDEAPPKESLPIPRFRRVLGRAENTWKGKEALVLPPTANRRQ |
| | | LEDPARRCRYTILKALYERPFRSWLIARAPEEVNAWIDRAIERTTRAAK |
| | | DMNAKRGEDDKRSVIAAKAESLPRLSGERGIGDFFFDLSSATASEMRV |
| | | QRGYGHDGEAAKEQAGYIDDLLCDVVALAFDAWLRNPQANGRPLTF |
| | | ICDLKPETPLPAAPKCTLQEIGSAAEPVRPEDWQAALYLLLHLVPVGEA |
| | | GRLLHQLAKWTVTSRLADDLLNANVTDDPSKAERTADEEDLKRLVHT |
| | | LIQHLDMHDAKFEGGDALTGCEPFAALFASRPGFARIFPAEADERLDR |
| | | RVPKRGLREIMRFGHHGLVASFAEDTRITDKEVGDYLRLEIEERPDNVA |
| | | ALQARKEEAHERWVKAKEKRKTVDPKHLEDYVTALCGIARHRRLASR |
| | | VTLTDQVQVHRLLMTVLGRLVDFSGMFERDLYFAMLGLLDEKGARP |
| | | DEVFSGPIDEPKSRLALLANGRVLAALREQIPHSKDLAEELRKDLERLFG |
| | | MDCSGIRLLEADERGDTCLRDIRNDLSHFNLLHDDSFALDLTTLVNRTR |
| | | GLMSYDRKLKNAVSKSIKELLAREGLTLSWDMTDRHDLENARIGAKP |
| | | AVHLGGRKLAFRGGDRRPEPVRENLHSPTHLEAVARLFGGKVVEEDD |
| | | VTNLDLSSIDWAAEPHNSKETHRHRPAGPRKSPPKRRAYHAPR |
| 50 | cas13a | MRIIKPYGRTLVEHDGAGERKRVLTLRPDHDSKLDIEAFARDHDELVV |
| | | AQWVSTIDKIAAKPGPRKGATEEQRAFRDRIGKAAWALLVRNALLPG |
| | | LADADRADRLAKIWRRKIAPYGDLRPNERPASAKGRWYGAFAGEAD |
| | | VADVDAGEIAAKIHEHLYDAEYRISGDGRKPDGCIAARARSIAVNVLRP |
| | | ADSSACGQPEWSDRDLQAYRVADVAKQIWDAALSRENGRDGAGTK |
| | | RVTNSVAGGVLFEHWARIFPGPDGKALSIREAIEKEPGLFALHMAVKD |
| | | CYARILKHHKKKAPGRRERENGDVSPIRKVLPRDMDELFARIISGRGNR |
| | | DLNALVRLGKVIHYTASDPNADHPESITENWPGDLAGSHYWTSAGQ |
| | | AEIKRNEAFVRVWRHVVVLAARTLTDWGDPHGEIGSDILGKANDAT |
| | | GAKFDEAAFNRKCALLFGKRASHFTAAPDLAFKKAVLKTAIKGMAALR |
| | | HKSFHFAGRGGFVKALEGIGGLNEIDRFPDVTRALRTLLVEDIEDQSRQ |
| | | VRATMVGAHFGVYLSKGQVEAIYRAVTGAEPGSLPLPRFSRVLRRAKG |
| | | AWEAEDVLPPPVNRLDLEQRGRLCQYTGLKLLYERPFRRWLEGRSAA |
| | | KLNGFIYRAVTRASDAARTLNTKESDDWRDIIVARAEKLGKVPDGGDI |
| | | HGFFFELSAETASEMRVQQAYESDGERARQQAEYIEDLKCDVVGLAY |
| | | RSFLETEGFDFLRTLDPEAAIAEAHRFDPAELPDPAVDTDAEDWEAVL |
| | | YFLVHLVPVDEIGRLLHQMRKWDLLAHDRTAPVADGGQARLVDKVQ |
| | | RVFTLYLDLHDAKFEGGEALTGIEPFRKLFEESDGFDTIFPPQQGYEEDR |
| | | RVPLRGLREIMRFGDLPPLLSIYGRRPATKSNIERYRRAEVADAGGRSEI |
| | | ARLQARREELHAKWVEAKKEGLGPEDRRAYVEALAEIVRHRHLAAHV |
| | | TLTNHVRLHRLMMAVLGRLADFSGLWERDLYFATLALLHRAGKTPRE |
| | | VFENEGIDLLRNGQIVYALRKLNGSSNASALRSGLFPHFGSAFKRGDPI |
| | | GGIRNAFAHFNMLRAAQPPNLTECINRARQLMKHDRKLKNAVSKSVI |
| | | DLLAREGLNIAWAVHTRAGAHDLAEAVLSSRQAQHLGKLRLFPVSGD |
| | | GRDGKGFFIMEDLHGADFVEMAAELFGGRVSDRWRGKGCVSELRLD |
| | | SIDWSRQREQKKHGGGKKPTGRARKANRGHKNRHRRA |
| 51 | cas12f | MSARNIKVKIDTKGNPELRLGLWKTHQVTNEGVKYYTEWLIKLRQQD |
| | | IYRQSREDASPRVIISASDLKADLLCHARQLQKERLPRITGSDAEILGTLR |
| | | QVYELIVPSSVGKSGDSKTLARKFLSPLTDPGSAGGRDQSASGRKPTW |
| | | MKMKSEGNPRWEETFRKWKDRKDNDPTPLVLNQIADYGLLPLIPLFT |
| | | DVGENIFDPKSKSQFVRTWDRSMFQQAIERLMSWESWNQRVRRE |
| | | WEALNQKHSAFYREQFTADPDAALYRVAQSLEEEMRKEHQGFASDA |
| | | PEAFRIRRVALKGFDRLLERWQKTLGKNGQSATLLDDIRRVQSDLGDK |
| | | FGSAPLYQKLLDERWQRLWAVDPTFLQRYAAFNDLTQRLQRAKRVA |
| | | NLTLPDAVAHPIWSRYEGANASSGNRYHIHLPTKGQPGSVTFDRILW |
| | | PDGNGGWYERKRVTVFLRPSHQVDRIHEAPTDSVVDNFPLVVEDQS |
| | | ARTILRASWGGAKLEYDRNRLPRQLKKGVPDSIYLSLTLNLDTNKPSGL |
| | | FHTQQNGRVWIRKDVLMQYYNETPGDNVQFKPLYVMSVDLGIRSAA |
| | | AVSIFSVQLKAGIEEHRLTYPVADCPGLVAVHERSVLLTMPGERREQW |
| | | DRRYEQQRQGLRELRTDMRGMNDLLRGAYMDGDRREEFLARLSKLE |
| | | ETSPELWGPVYRSLNDSKVASATEWERLVVYCHRQVEQSLSSRIQNLR |
| | | SGRSAYRMSGGLSLDHVQDLERIRGIIASWTNHPRIPGSVVRWQQGR |
| | | SHTVALGRHILELKRDRVKKVANYLIMTTLGYAYDSKRARGEKWVRRY |
| | | PACHLMVFEDLTRYRFRTDRPRSENRQLMRWTHQELIAVTGIQAEPH |
| | | GISVGTMYAGFSSRFDAVTKAPGVRGATVRQILRTRGMVRLKEIAAD |
| | | VGIDINTLRPHDVLPTGDGEYLLSVVRHGESYRLKQVHADINAAHNLQ |
| | | RRLWTQDEVFRVSCRLALNSGRWAMPPPSYNKRYGKGFFEKGDNG |
| | | VYIWKTGGKIKISDTLEEDMDIPEDTAELLRGNSVTLFRDPSGTIAGGN |
| | | WLEAKEFWGRVNSLVNKGVRDKILGGIPVDNSSAHAE |
| 52 | cas12f | MPMIKITECVTWGTTCDGLWDARPHLEVRRSWSPPVQGGRTNRLD |
| | | APPASVTLNIHGRVEHPRDADALSVAPLRVRHMFERTTTKAAFLSPLD |
| | | LRPTQATDLERFAGTTRWAFNWANALLEAHHQAYEGRRQQAARHL |
| | | FGLGPEQLDELRVLANGTRDENGKKAKGDPVKRREYESIQKATKKAVS |
| | | EENKALGAEMKLWDEHRSLVVHKGRPLLTPGDEPALDAPPLAHRLYA |
| | | RRVELAGIQKTDPDYYAEQRKKEREAITPNVVAMKRDLMAKGAYFPS |
| | | EYDLQYIWRTVRDLPKEEGGSPWWPECPTILFYDGINRARTAWKNW |
| | | MDSASGARKGPPVGMPRFKSKYKAKDTFTITNPNRSVIKFETYRRIAIT |
| | | GIGSMRLHRGAKLLARRIAAGQAEITSATISRSGTAWYVSVLCTVHTTA |
| | | RTAPSKAQRSRGAVGVDWGVRALATTSKPIALTPGKPASRTVPAEKY |
| | | GAAMSQKIARAQRQLARMPKGSSRRRKAARHVADLQHLVAQRRAS |
| | | SVHQLSKALAQSFEIVAIEGLNVRGMTKSAKGTVENPGKNIRQKAGLN |
| | | RAILDATPGELKRQLEYKTKKYGSRLVELDTWYPSSKTCSRCGWVHPK |
| | | LKLSMRTFRCQQCGLVEDRDFNAAVNIERQGITHIVKENEGTDDREE |
| | | G |
| 53 | cas12f | MSTPMGWTAVNGGDATSPTTRVSSPPGEPRTGACPRAAAADATRA |
| | | ESSPRRTSSPARPGERHARARTSRYPIPNTYVVDRPSAEGDRHGQSSL |
| | | DCGPCPVRRSGALHQSSQAAHRRSMTGAKQKTPIRVVRFSIDHSALT |
| | | PAQVVAFARHAGAARQTWNWALGRWMDWRNNTKFYVDYKVFKA |
| | | AGMGPGLSTDDLIQVIERAVSIRQDDKWMDAAWDEARQIHGEWD |
| | | QFQKASTLQSLYLAGAQEPFDPSRDDGINPYHWWVTEGDKSGLPKA |
| | | ERHNVNSGATYTAPLRAFEEAVGRFYKLPGKKGTPKFKSKHDDEQGF |
| | | CIQRLTETGLSPWRAIEGGHRIKVPSIGSIRVVQSTKRLRQLIKRGGKTT |
| | | SARFTRRGGKWFVSVSVAFDLSAPRVQRPARLSRRQRAGGSTGVDLG |
| | | VNRLATLSSGDQFPNRRLLRKSMAEIKRLQRKFDRQHRAGSPECFNE |
| | | DGTHKKRCRWGREDGPAMSRSAQTTKRQLRRIHDLTARRRAGVLHE |
| | | ITKDLATRFELIGVEDLNVAGMTAKSKPKPDPDRPGHFLPNRRAAKAG |
| | | LNRAILDVGFYEFKRQLGYKTEWYGSTMQMVHRYAATSKTCSGCGW |
| | | VKPKLTLAERTFNCTQCGLAMDRDHNAAVNIRALALEGAAPMEREQ |
| | | PAPVGAAEKRHRDPVSHRRRPKSLAPCESTRPVRDLSPPATQEETA |
| 54 | cas12f | MAQAEAPRRLRAYKFALDPTEAQLREFEQHAGSARWAYNHANAILS |
| | | RYSDTLRNRWNAWIAQHHGLSREQLYALPDRERTAIQAAARAAVKA |
| | | ENAQLAAELRIIDDHRKRVTHKGKPSVEPGEQPAEDAPERAYQLWRE |
| | | RVELARLHAEDPQAYRAERKRILDEIRPLVNATKRKLIEQGAYRPTAM |
| | | DISTLWREIRDLPPDEGGSPWWPEVSIYAFTSGFAHAETAWKNYLESL |
| | | AGRRAGRPVGKPRFKKKRRSRRSFTLYGSVKLVTYRRIQVPSIGSVRLH |
| | | GSAKRLHRALERRGGIIKSITISQGGHRWYASVLVDELDITPGRETQRG |
| | | PSRRQRDRGAVGVDLGVHHLVALSDPNEKTLDNPRHLRKARKRLLKA |
| | | QRAMSRRRGPDKRTGQEPSRRWVKARNRVARLHHELAVRRAGHLH |
| | | EITKRLATSYELVAIEDLNVAGMTRSARGTIDQPGRGVRAKAGLNRSIL |
| | | DTSPAEFRRQLQYKASWYGATVAVIDRWAPTSRTCSSCGAVKAKLSL |
| | | AERTFFCEHCGMELDRDINAARNILAFAQSAYPGEGKALNACGGSVS |
| | | PGSQSVVQAGADEAGRPARKPRRSSRGSDPPATPTTRA |
| 55 | cas12a | MTSSSPTQRAYTLRLKSAAQGDKSWAEKLWDTHEIVNKGARAFGDW |
| | | LLTLRGGISHKLENLNDKETGEEGKKRRRILLALSWLSVESKDFAPEKYI |
| | | VEKDGEDKHRTKEALEAILKSRNLEDEEVESWVNDCKDSLTSSIRDDA |
| | | VWVNRSRAFDDAVRKIGDSLTREEIWDVLGRFFGKKEAYLAPRTIDEK |
| | | NGKTKKEEPKDLARKAGGWLSKRFGKGKGTDFSKLSKVYSEIVKWAEE |
| | | PRKSEPRTLANLASALKEDSLQGILNLIKNSGSKSGTRNFLEEIGEGEVS |
| | | KENLAILKAKAEGNRNYCKKEIGGKGRREWSDRILKSIEETLDGKFTYL |
| | | QEKGPARHWEFAVMLDHAARRISAGHTWIKLAEARRRNFEEDSQKI |
| | | NEVPENARQWLETYREDRSKSSGAIEGYLISKRAVTEWETVVKAWKN |
| | | CKTEEDRIAAAGALQDNLGIDQFGDINLFRALASEDVRCVWQVDGKP |
| | | DANILLNYVAATKAEFDKRRFKVPAYRHPDPLLHPVFCDYGNSRWEIR |
| | | FDVHEVNRTGKKAKQNKKTIETADVHGLKMDLWTGSKIENVSLRWQ |
| | | SKLLEKDLAVKQLDGKEDGKKEVSRASRLGRAAVGAGWETPVSASSV |
| | | FAQKHWNGRLQASRKELSRIARRVKTRGWDEKANSMKKNLKWFITF |
| | | SPKLKLQGPWISYVDNSEDKRPFTFTSKGEPILDEVFSIENKNRKGRAR |
| | | LILSRLPGLRVLSMDLGHRHAAACAVWETLSSRQLEDACAEGGYDKP |
| | | APDAMYHHIKSNRGKRVIYRRIGADELSDDSIHPTPWARLERQFLIKLQ |
| | | GEERKARMATADEIWEVHELERALGRKTPLVDRLTKSGWGSDSGTPR |
| | | QRQLLGELNQWGWEPDEAQENSEDDEITSRESLLVDKLMSRTVDTV |
| | | RKGLRRHGNRARIANFLVAREKTVPGGQMDTLNNEGRKEIIADALAF |
| | | WYELANGGEWKDTEALDWWKIHIEPELSVEELPDIAGTGIAPKERKR |
| | | KKKELKEKLKPVAERLLTSGAKKLSDQWCERWKQDDKEWQKTLRWL |
| | | RDWILPRGVRGKSELIRNVGGLSLDRLTTIQSLYQAQKAYFTRITPKGIQ |
| | | MDKDKPLTAVMNFGGHILNDLENMREQRVKQLASRIVEAALGVGRV |
| | | KIPKKSKDPKRHYERVDAPCHAVVIENLTNYRPEETRTRRENRQLMT |
| | | WCSGKVKKYLSESCSLHGLFLWEVPPSYTSRQDSRTGSPGIRCEEVSVE |
| | | KFFKTPFRQREVARAEEKDSKNKASAYEQYLIDLKERWKSRGEETALLR |
| | | IPRKGGEIFVSANSNSPASKGLQADLNAAANIGLKAITDPDWSGSWW |
| | | YVPCSSKDFVPIKDKIGGSRAFENITTPMPNPDDAKEATGKKRSGKKEII |
| | | NLWRNPACSPLERDEWERTAKYWNMVEYHVIKRLKRQMG |
| 56 | cas12a | MKNFQDFTNLYELSKTLRFELKPIWGTKKLIEEKNILKLDKKKRENYEKV |
| | | KPYFNKIHQEFINFALRNPNFDFSQFEEKYLNWLKDKKNKDLLKEKESI |
| | | DKIFLEKIWKLFENSVKDFLKENGFESIVKSEDQNLKFFRRKEIFEVLQEK |
| | | YGSELETQMVNKDWEIKSIFNGWEKWLWYFDKFFNTRDNFYKTDW |
| | | TSTAIATRIIKDNLKIFLENTIIFEKVKNKKIDFSEVEKNFSVSIDTFFEINNF |
| | | NNCFLQDWIDFYNKVIWGETLENWEKLKWLNEIINKYRQDTGEKIPY |
| | | FKKLQKQILSEKDWVFIDKIEDDGGFYEVLKNFYKNAAEKEWFLKNIFE |
| | | NFYTISDKNLEKIYFNKIAFNTISHKFWSALEFERILYEEMKKEKADWIKF |
| | | EKKENKYKFPDFIQIIFIKRSLENYDSENLFWKERYYKSEENVDWFLEKN |
| | | NNNIWEQFCKILNFEFLNILKRRIIDEAWEEYEVWFEISKNILWEKLENF |
| | | ELNQENKWIIKDFADYSLALYSFWKYFAVEKWRNWDLNIDISDDFYG |
| | | WEDWYIEKFYNTGYDEIVKPYNLMRNYISKKPWEDSKKWKINFETSSL |
| | | LSWWDKNLESNWSYIFQKWNKYYIWIINWSKPAKEVLEKLYSWNGE |
| | | KIKRFIYDFQKPDNKNTPRMFIRSKKDSFSPAVGKYNLPVEDILEIYDN |
| | | WLFKTENKDNSNYKESLSKLIDYFKLGFSKHESFKHFNFVWKDSKEYEN |
| | | IADFYRDVEKSCYQITSEFLDFEELKKLTFKKHLYLFQIYNKDFELDESLQ |
| | | KNWYNFRDEWQKNIHTKYFEALFLEENILRKSWAVFKLSWGWEVFF |
| | | RKESIKAEKEKRKNIEVTKNRRYTEEKYFLHFPIQVNFKNEISWNFNQEI |
| | | NKFLANNPDINVIWIDRWEKHLAYFSVINQKWEILESWSFNKIENYNK |
| | | NWEKLLFPEREIKEIHKDWSLIDLELVETWRKVDYVDYKLLLEYKERKRL |
| | | LQRQSWKEVEQIKDLKKWYISALVRKIADLIIKHNAIVIFEDLNFRFKQI |
| | | RGWIEKSIYQQLEKALIDKLNFLVNKNEINLEKAGSILKAYQLTVPVDSL |
| | | KEIWKQTWVIFYTEAAYTSKIDPIKWWRPNLYLKKQNAEINKENILKFD |
| | | NIIFNSKENRFEFTYDLKKFFWKDSKFPAKTVNTVCSCVERFKWNRNL |
| | | NNNKWGYIHYENLTDWKLANKEQKEDEFSNFKELFEKYFIDINWNILE |
| | | QIKNLDTKNNEKFFSSFIDLFTLVCQIRNTNQNAKWDENDFILSPVEPF |
| | | FDSRKSQNFWKSLPKNWDENWAFNIARKGLIILNRISENPEKPDLLIFN |
| | | ADWDNFARNI |
| 57 | cas13b | MTEQNEKPYNGTYYTLEDKHFWAAFFNLARHNAYITLAHIDRQLAYS |
| | | KADITNDEDILFFKGQWKNLDNDLERKARLRSLILKHFSFLEGAAYGKK |
| | | LFESQSSGNKSSKKKELTKKEKEELQANALSLDNLKSILFDFLQKLKDFR |
| | | NYYSHYRHPESSELPLFDGNMLQRLYNVFDVSVQRVKRDHEHNDKV |
| | | DPHRHFNHLVRKGKKDKYGNNDNPFFKHHFVDREEKVTEAGLLFFVS |
| | | LFLEKRDAIWMQKKIRGFKGGTEAYQQMTNEVFCRSRISLPKLKLESL |
| | | RTDDWMLLDMLNELVRCPKSLYDRLREEDRARFRVPVDILSDEDDTD |
| | | GTEEDPFKNTLVRHQDRFPYFALRYFDLKKVFTSLRFHIDLGTYHFAIYK |
| | | KNIGEQPEDRHLTRNLYGFGRIQDFAEEHRPEEWKRLVRDLDYFETGD |
| | | KPYITQTTPHYHIEKGKIGLRFVPEGQLLWPSPEVGATRTGRSKYAQDK |
| | | RFTAEAFLSVHELMPMMFYYFLLREKYSEEASAEKVQGRIKRVIEDVYA |
| | | VYDAFARDEINTRDELDACLADKGIRRGHLPRQMIAILSQEHKDMEEK |
| | | VRKKLQEMIADTDHRLDMLDRQTDRKIRIGRKNAGLPKSGVIADWLV |
| | | RDMMRFQPVAKDTSGKPLNNSKANSTEYRMLQRALALFGGEKERLT |
| | | PYFRQMNLTGGNNPHPFLHETRWESHTNILSFYRSYLKARKAFLQSIG |
| | | RSDREENHRFLLLKEPKTDRQTLVAGWKSEFHLPRGIFTEAVRDCLIEM |
| | | GYDEVGSYKEVGFMAKAVPLYFERACKDRVQPFYDYPFNVGNSLKPK |
| | | KGRFLSKEKRAEEWESGKERFRDLEAWSHSAARRIEDAFVGIEYASWE |
| | | NKKKIEQLLQDLSLWETFESKLKVKADKINIAKLKKEILEAKEHPYHDFKS |
| | | WQKFERELRLVKNQDIITWMMCRDLMEENKVEGLDTGTLYLKDIRT |
| | | DVQEQGSLNVLNHVKPMRLPVVVYRADSRGHVHKEEAPLATVYIEER |
| | | DTKLLKQGNFKSFVKDRRLNGLFSFVDTGALAMEQYPISKLRVEYELAK |
| | | YQTARVCAFEQTLELEESLLTRYPHLPDESFREMLESWSDPLLDKWPD |
| | | LQREVRLLIAVRNAFSHNQYPMYDETIFSSIRKYDPSSLDAIEERMGLNI |
| | | AHRLSEEVKLAKEMVERIIQA |
| 58 | cas13b | MESIKNSQKSTGKTLQKDPPYFGLYLNMALLNVRKVENHIRKWLGDV |
| | | ALLPEKSGFHSLLTTDNLSSAKWTRFYYKSRKFLPFLEMFDSDKKSYEN |
| | | RRETTECLDTIDRQKISSLLKEVYGKLQDIRNAFSHYHIDDQSVKHTALII |
| | | SSEMHRFIENAYSFALQKTRARFTGVFVETDFLQAEEKGDNKKFFAIG |
| | | GNEGIKLKDNALIFLICLFLDREEAFKFLSRATGFKSTKEKGFLAVRETFC |
| | | ALCCRQPHERLLSVNPREALLMDMLNELNRCPDILFEMLDEKDQKSFL |
| | | PLLGEEEQAHILENSLNDELCEAIDDPFEMIASLSKRVRYKNRFPYLML |
| | | RYIEEKNLLPFIRFRIDLGCLELASYPKKMGEENNYERSVTDHAMAFGR |
| | | LTDFHNEDAVLQQITKGITDEVRFSLYAPRYAIYNNKIGFVRTGGSDKIS |
| | | FPTLKKKGGEGHCVAYTLQNTKSFGFISIYDLRKILLLSFLDKDKAKNIVS |
| | | GLLEQCEKHWKDLSENLFDAIRTELQKEFPVPLIRYTLPRSKGGKLVSSK |
| | | LADKQEKYESEFERRKEKLTEILSEKDFDLSQIPRRMIDEWLNVLPTSRE |
| | | KKLKGYVETLKLDCRERLRVFEKREKGEHPVPPRIGEMATDLAKDIIRM |
| | | VIDQGVKQRITSAYYSEIQRCLAQYAGDDNRRHLDSIIRELRLKDTKNG |
| | | HPFLGKVLRPGLGHTEKLYQRYFEEKKEWLEATFYPAASPKRVPRFVN |
| | | PPTGKQKELPLIIRNLMKERPEWRDWKQRKNSHPIDLPSQLFENEICR |
| | | LLKDKIGKEPSGKLKWNEMFKLYWDKEFPNGMQRFYRCKRRVEVFD |
| | | KVVEYEYSEEGGNYKKYYEALIDEVVRQKISSSKEKSKLQVEDLTLSVRR |
| | | VFKRAINEKEYQLRLLCEDDRLLFMAVRDLYDWKEAQLDLDKIDNML |
| | | GEPVSVSQVIQLEGGQPDAVIKAECKLKDVSKLMRYCYDGRVKGLMP |
| | | YFANHEATQEQVEMELRHYEDHRRRVFNWVFALEKSVLKNEKLRRFY |
| | | EESQGGCEHRRCIDALRKASLVSEEEYEFLVHIRNKSAHNQFPDLEIGKL |
| | | PPNVTSGFCECIWSKYKAIICRIIPFIDPERRFFGKLLEQK |
| 59 | cas13b | MESIKNSQKSTGKTLQKDPPYFGLYLNMALLNVRKVENHIRKWLGDV |
| | | ALLPEKSGFHSLLTTDNLSSAKWTRFYYKSRKFLPFLEMFDSDKKSYEN |
| | | RRETAECLDTIDRQKISSLLKEVYGKLQDIRNAFSHYHIDDQSVKHTALII |
| | | SSEMHRFIENAYSFALQKTRARFTGVFVETDFLQAEEKGDNKKFFAIG |
| | | GNEGIKLKDNALIFLICLFLDREEAFKFLSRATGFKSTKEKGFLAVRETFC |
| | | ALCCRQPHERLLSVNPREALLMDMLNELNRCPDILFEMLDEKDQKSFL |
| | | PLLGEEEQAHILENSLNDELCEAIDDPFEMIASLSKRVRYKNRFPYLML |
| | | RYIEEKNLLPFIRFRIDLGCLELASYPKKMGEENNYERSVTDHAMAFGR |
| | | LTDFHNEDAVLQQITKGITDEVRFSLYAPRYAIYNNKIGFVRTSGSDKIS |
| | | FPTLKKKGGEGHCVAYTLQNTKSFGFISIYDLRKILLLSFLDKDKAKNIVS |
| | | GLLEQCEKHWKDLSENLFDAIRTELQKEFPVPLIRYTLPRSKGGKLVSSK |
| | | LADKQEKYESEFERRKEKLTEILSEKDFDLSQIPRRMIDEWLNVLPTSRE |
| | | KKLKGYVETLKLDCRERLRVFEKREKGEHPLPPRIGEMATDLAKDIIRM |
| | | VIDQGVKQRITSAYYSEIQRCLAQYAGDDNRRHLDSIIRELRLKDTKNG |
| | | HPFLGKVLRPGLGHTEKLYQRYFEEKKEWLEATFYPAASPKRVPRFVN |
| | | PPTGKQKELPLIIRNLMKERPEWRDWKQRKNSHPIDLPSQLFENEICR |
| | | LLKDKIGKEPSGKLKWNEMFKLYWDKEFPNGMQRFYRCKRRVEVFD |
| | | KVVEYEYSEEGGNYKKYYEALIDEVVRQKISSSKEKSKLQVEDLTLSVRR |
| | | VFKRAINEKEYQLRLLCEDDRLLFMAVRDLYDWKEAQLDLDKIDNML |
| | | GEPVSVSQVIQLEGGQPDAVIKAECKLKDVSKLMRYCYDGRVKGLMP |
| | | YFANHEATQEQVEMELRHYEDHRRRVFNWVFALEKSVLKNEKLRRFY |
| | | EESQGGCEHRRCIDALRKASLVSEEEYEFLVHIRNKSAHNQFPDLEIGKL |
| | | PPNVTSGFCECIWSKYKAIICRIIPFIDPERRFFGKLLEQK |
| 60 | cas13b | MDTPNFSERIPVSLQSHPYYFAHYLNMARHNAYVILEYVNRELIKPGK |
| | | NLDEDNLIQSTVLKDGYFDRKPDELSHRNRLLVQHFPFLREAENEGAR |
| | | TCNPVSYKLKTALAALNQWRNNASHYPLNQNHEKDFDLQPFFSFAIE |
| | | ACKKRMREVFQPDDFYLLETNEKQFYTLHNENGFTEKGLYCFICFFLEK |
| | | KYAFQFLAGIKGFKNTTDNKFRATLETFTEHCCRLPKPKLDSSDIKLDM |
| | | LGELSRCPAPLFDLLDIEERKKFIREPEEVKPDESGDREEVQQVLMKRY |
| | | DDRFPYFALRYFEEKNLLKGISFHIHIGRWIKSEHTKKIMGAERDRRLLK |
| | | DIRTFGELKEFSPEHAPDYWLRDGITPDDVDQFSPQYRIVGNRIGIKLN |
| | | YNGHNRWSVPDKEINVKPDAIISTYEFLNLFLYEHLYQKKLTGLSPAEFI |
| | | QDYLDRFNNFLSEFKAGHIRPVGDFSLEKRRGQGDEPDLTARRKSLQK |
| | | ELDRFVLKGKDLPDKIREYLLGYKQKSEKKQAKWILGGMIKETVYWRN |
| | | KAEQSPEKMRSGDMAQQLARDIIFLTPPHTVKEHKQKLNSLEYDVLQ |
| | | YALAYFSSNREKLYSFFKEHQLTVKGDRAHPFLYKIRLDECQGILDFFIVY |
| | | MQQKEKWLGWLDRNLKSPRLNEEEFFNTYSYFIKTDTKRAIEMDYES |
| | | CPNYLPRGIFNEPIAKALQKAGVKIKDEDNASYALSVYSNGKTQPFYNK |
| | | ERYYNKGIFRMEELPEKLQPKELLGKIQWTIKSSGKDTEEFRSLQNLKN |
| | | RILNTEKEIRYVQSTDRALWIMVADLFPETFELRPDDLECIGHDLSDDLL |
| | | SRPYQMKEKVYNYTITDYLPIKRYGEFRRFLKDRRLENLLTYFEEGVPLH |
| | | REALVAELEAYDLQRKNLLEIIYRFEKLVFDRHRHELTFSGEGENQYVN |
| | | HWDYLDFVARKYGLSAEVKELNSERFTELRNKMLHNQIPYQLWIKEAl |
| | | AAREENTVCGRIMGMIGEIYERMTTEIEKQMQV |
| 61 | cas13b | MFDNEQKNLEKEPYWGVFLNQARLNAYIALRDISERLEENAADEDSLS |
| | | EWPVLKYLDNDTDAVKSRRIFDLVEKHFSMLKIIYGGEKEGDLVKRSKE |
| | | YKIILKCLFRALNFYRNKFCHMYSGNRARKYNEKELIKYLEDCFDASVRK |
| | | IKELRRLDEKDVLHLRRKIAEGKDANKRVIDNPQFRYPFKNEKGELNEK |
| | | GLYFLASIFLDKKEAHEFLKKQEYFKNDSEPKYRATLESFYHYRIKLPRPV |
| | | lESDVDKNGLALDMLNELKKCPKELFDLLSKEQQEKFRVVDSEDADEE |
| | | GNEILMRRYSDRFPYLALRYCDENQVFERIRFQIDLGRYYFKFYPKETID |
| | | GKTQQRSLDKRLKIFGRIKDVKSKVEQEWSGIIKSPDTIEENPNEPYKLK |
| | | TTPRYNIVDNQIGFVITGDKNLPDVKRPDGRIELEKPDGWLSIYELPGM |
| | | LFHGLKYGFDKTERMIKIYIEKQRKICKEICEKGTITPDDGESMPEALKG |
| | | GAKAAKRNYSEKKLERMLQDTEQRIRAIQTTQKRMDEPGNKPGKKKF |
| | | FDIRAGKLADFLARDIMALQRFDPAKHGKDKLTAINFQVLQATLAFYG |
| | | AKKDVIEDMFKGIGLLEGDNPHPFLNQIDPAQYNSIAGFYQAYLQKKR |
| | | SYLEDYRKEEEYDEQFLRPKRQRYAQEKREIKTVARQLLDNPVNVPKN |
| | | FFKKEIEEFVFSQDPSLKKSKMNTAYMIQALFEKHYGRQQPFYSYNRT |
| | | YPVVSKAIEYGKKGKNKKIAKVLMAIEPKLNYMEIKKIVNEMPDGQYE |
| | | PENLKRNLYEGYKDYEKDERIIRRCKVQDVVSFMMVEETLKDQLDFN |
| | | GNVLTLEKITPWEASPFKKPVLCHTIISIPFNTKGGHTDKDYVDFIKNNF |
| | | EGSYDCEPNKIILKYKVTSKDTKLKDIGKYRMYSHDRRLPGLLIWKYRP |
| | | NDQNGNEIKFTEIEQEIKAFERRRIEIAQCLYTLEKKVIDSWFTQDELGE |
| | | EHIPFNKVIDVIKAKMPNFEDKCNVLLKIRNAINHNQFPVYEQAIQTAP |
| | | GKEIAGKMLRITESYIEQIMAKIDPDFGRTEDAESSR |
| 62 | cas13b | MDTPNFSERIPVSLQSHPYYFAHYLNMARHNAYVILEYVNRELIKPGK |
| | | NLDEDNLIQSTVLKDGYFDRKPDELSHRNRLLVQHFPFLREAENEGAR |
| | | TCNPVSYKLKTALAALNQWRNNASHYPLNQNHEKDFDLQPFFSFAIE |
| | | ACKKRMREVFQPDDFYLLETNEKQFYTLHNENGFTEKGLYCFICFFLEK |
| | | KYAFQFLAGIKGFKNTTDNKFRATLETFTEHCCRLPKPKLDSSDIKLDM |
| | | LGELSRCPAPLFDLLDIEERKKFIREPEEVKPDESGDREEVQQVLMKRY |
| | | DDRFPYFALRYFEEKNLLKGISFHIHIGRWIKSEHTKKIMGAERDRRLLK |
| | | DIRTFGELKEFSPEHAPDYWLRDGITPDDVDQFSXPQYRIVGNRIGIKL |
| | | NYNGHNRWSVPDKEINVKPDAIISTYEFLNLFLYEHLYQKKLTGLSPAE |
| | | FIQDYLDRFNNFLSEFKAGHIRPVGDFSLEKRRGQGDEPDLTARRKSL |
| | | QKELDRFVLKGKDLPDKIREYLLGYKQKSEKKQAKWILGGMIKETVYW |
| | | RNKAEQSPEKMRSGDMAQQLARDIIFLTPPHTVKEHKQKLNSLEYDV |
| | | LQYALAYFSSNREKLYSFFKEHQLTVKGDRAHPFLYKIRLDECQGILDFFI |
| | | VYMQQKEKWLGWLDRNLKSPRLNEEEFFNTYSYFIKTDTKRAIEMDY |
| | | ESCPNYLPRGIFNEPIAKAVQKAGVKIKDEDNASYALSVYSNGKTQPFY |
| | | NKERYYNKGIFRMEELPEKLQPKELLGKIQWTIKSSGKDTEEFRSLQNL |
| | | KNRILNTEKEIRYVQSTDRALWIMVADLFPETFELRPDDLECIGHDLSD |
| | | DLLSRPYQMKEKVYNYTITDYLPIKRYGEFRRFLKDRRLENLLTYFEEGV |
| | | PLHREALVAELEAYDLQRKNLLEIIYRFEKLVFDRHRHELTFSGEGENQY |
| | | VNHWDYLDFVARKYGLSAEVKELNSERFTELRNKMLHNQIPYQLWIK |
| | | EAIAAREENTVCGRIMGMIGEIYERMTTEIEKQMQV |
| 63 | cas13b | MKTLGALSSHNYNNKKYYFSGLLNTAQYNFNLALQEVNDRLGKKGKN |
| | | PGKTMIKNIFDQKDSFSTQERAMYYLEEFFPWIFLVMKQSGINIPTEE |
| | | QETKLHKEEIQLIQEHLISLYELLDDLRNEQTHYMHDPVIIPEEVSKMLD |
| | | ALLLQILKNTRKKCKDDEYRTFIVKKYQEEFQKEIKVQVKDRFGKEKEKI |
| | | VTGEVKENYVINRCFRKWIQKEGEEETLRYSTVQEEQGKYVWSSSGFV |
| | | FFLSLFLRRKELEDVMNHVPYFKDSRKLLFYLTRKTFSSYCFRDLRKSLRS |
| | | DYSNDSLLMQMIEELYKCPGELYEVLLKEQKQEFIEDINEYYKDNPEFE |
| | | GSANEAQVIHPVIRKRYQDKFPYFALRFIDEYFNFPTLRFQLVLGEYVT |
| | | DRRTKELQGTALFTDRVISQRISYVGKLSEAEMNKKREGYTETGWKEY |
| | | PNPYYKIENNRIPLYIEFSKNEELIFKEKKFKYNTLAKWENREIDKRTGEF |
| | | NQVNKQRRITQLEEFKIDNPKKMKTPNVFLSIYELPALLHALLIEKKTEA |
| | | EIEDIIKAKIKKQLTEIAEGRRNLSGLPKGIKKMRNCNSDFEKKKLISDID |
| | | NEIKKGEKILEEVQQWLNPVINKKGTGKQENNKPFFSNTYRGKYATW |
| | | LAYDIKRFTGKDHIQNWKGYQFSELQTLLSLYTLRKEELKNFLEKDLQLT |
| | | SHPFLKEALKAVNLEDFMGAYLRGRQFFLEKAKKQIGIKGVKKSIFQYF |
| | | EERKYKIYSSNLDYWEELWKHPVNLDRGLFDERGTVYNKNKELNDLQ |
| | | NRAAWFSFAETNPKQQFYHFPRIYSDEDITKPVTDRYGKTKEKLILFKLS |
| | | PQKGFMEQIPSDLKKKYQEDKGKVEHPEVQKEKKYEEKKHPGINAFIK |
| | | NAYKNEQKIRRISRNDIFLYEMVKYMLNKISPATEFSSLDKVWLTRIERE |
| | | KQATEAREQSFKEKGDTSENKIRQDYLLSFPITLTLFNDIIKEKVKIKDIG |
| | | RFRKLEKDERVQTMISYYTSGLWKNDQPSLTIKELEAELESYHKIRLQEI |
| | | FKEVHKLEKEIYEFTPEEDKSKLLARESFPKFKYYISFYFIPKEDQEVFNEI |
| | | QFDKYKNLEQIPGRKPEYDPYYLLIFIRNKFAHNQLPAEPIYKTALTFLP |
| | | NNFNTLAEYYHKLFILLNNKNYNN |
| 64 | cas13b | MNILPAAPEKEKIAYSTATAPWFFGAFLNQARHNLFLTVNDLAIRLGE |
| | | KVIDYDDQLLNSNVVRMLVNEKASPLQLEILMKYLDRHLPFLIPMQVA |
| | | LKGHQGDASDNPVIGSPADYGAILSKLIVCLNAARNHFSHYHSTSGWS |
| | | GYNEVIEWMEHVFTRNIETVVKRFTLTEEEVQHLKKPVDKSPKGTIPPY |
| | | YFSFCKGDIWTDTGLAFFICLFLTREEAYLFLKKLRGFKRGEERFHKATLE |
| | | AFCVGSLKVPRERLESNNSPQSAFLDMCNELVRCPKSLFDLLEPEKQEL |
| | | FRRDPEPEDAEDNGIEEEEDQPQALLVRKENRFSYFALRYLDIAKAFPR |
| | | LRFGVDLGTYFFSVYPKTFAGIEETRQLSKRLIGYGKLEEFAREKRPEHIA |
| | | ALFRSKEEANAAPTEPFIRETAPHYHLDGNNVYLYMSGDGEAQWPAV |
| | | ELEEVTGKSYPRKLVKKSTLLPFAVLTVNELPALLFYHLLHKEKGAGDAA |
| | | ERVIINHMERVKRFFKALQDDKVDQVAGQPIRKPDVDADESLHMEY |
| | | DRRWKLLKKKLSEYQLRASYIPEKIINYLLNIEAVDLGDKAMAQLKNLQ |
| | | RQAQDDIAAIERRMEHLMKKGADGRKTLKVGNLAQQLAEDMLQM |
| | | QPVQIGTDGEPVPASKANNLAFRLLQSHLAYFAENRHNLPAVFEACGL |
| | | IGASNKHPFLDNINIESCKGVVDFFILNFRNKLDFLDRCLQEGEWHRYH |
| | | FISAAKLKSGAKVTIKKYLNEAFESKGRNHIPFTLPPSLFLDASLDWLAKF |
| | | GDGKAKKVLAENEYVNSVFLIRRLFADGGLQPFYAWKREYRLFEKKAG |
| | | KAVFLDEAGRMRKADKIGIEVERHREFLARPVKKGKQYDIKKAAAEQF |
| | | LRSYRFYLQEEKYIRLLAAQDMLLFRCICDLLTYHVGDIGLEELAEAKAG |
| | | TFSLANITPEKTETAKSLLNYRPAGGVVLDRHFYATDEKGAFVKQEGKL |
| | | VPGGQVRIFDNTLKIKNAGNFRKLLKDRRMNNLFFYFKQHADEPVVL |
| | | HRMVLENELRAYDRMRLKVLPVIAEFEKKLYQHCTDVEKERLVVNGS |
| | | MHHRCYLDVYREKYQPDWGWEAAGNLLRIRNAFVHNQFPLMEGD |
| | | GFKLEVAHWKKINADFVPSEQGSSLGYGIIDRLGQLAVEGYEGLIKNIH |
| | | V |
| 65 | cas13b | MGEEEQAHILENSLNDELCEAIDDPFEMIASLSKRARYKDRFPYLMLRY |
| | | IEEKNLLPFIRFRIDLGCLELASYPKKMGEENNYERSVTDHAMAFGRLT |
| | | DFHNEDEVLQQITKGITDEVRFSLYAPRYAIYNNKIGFVWTSRSKKKSF |
| | | PTLKKKEGEGHRVAYTLQNEESFGFISIYDLRKILLLSFLDEGKNIVSGLF |
| | | KQSKANWENLSENLFDAIRTELQKEFPVPLIRYTLPRSKGGKFVDPKLA |
| | | DKQEKYESEFERRKEKLSEILSEKGFDLSQIPRRMIDEWLNVLPTSKEKK |
| | | LKGYVETLKLDCRERLRVFEKREKGEHPVPPRIGEMATDLAKDIIRMVI |
| | | DQGMKQRITSAYYSEIQRCLAQYAGDDNRRHLDSIIRELGLKDRKKGH |
| | | PFLGKVLRPDLDHTEKLYQRYFKEKKEWLEATFYPAANPKRVPRFVNP |
| | | PAEKQKELPLIIHNLMKERPEWRDWKQRKNSHPIDLPSQLFENEICRLL |
| | | KDKIGKESSGKLKWNEMFKLYWDKEFPNGMQRFYRCKRRVEVFDKV |
| | | VEYEYSEEGGNYKKYYEALINEVVRQKISSSKENSKLQVEDLTLSVRRAF |
| | | KRAINEKEYQLRLVCEDDRLLFMAVRDLYDWKEVQLDLNKIDNMLGE |
| | | PVSVSQVIQLENGQPDAVIKAECKLKDVSKLMRYCYDGRVKGLMPYF |
| | | ANHEATQEQVEVELRHYEDHRRRVFDWVFALEKSVLKNEKLRRLYEK |
| | | SQEGCEHRRCIDALRKATLVSEEEYKFLVHIRNKSAHNQFPDLEFGKLT |
| | | PNVTSGFCECIWSKYKAIICRIIPFIDPERRFFGKLLEQK |
| 66 | cas13b | MRIPKLIEEHKSVFGAYSTMALSNVETVLNHIAERAGLDGYERDRGPG |
| | | VEDYWEHPVMQCLCRKDKPRSIPSDVLLDVRNRLFRSFPFLKIMAEN |
| | | QRDYRNAKGKVECVEINESDIFVVLNNSFRVLKAYRDTCTHYLIENRIW |
| | | EDNSPMLMYNECPLAAMVNQYYTAALRVTKERYGYETRDLTFIQKRR |
| | | FKQEPEKEASGNVKKKAVPDLAFFLSLVALNGDGRKWLHLSGWGVVL |
| | | LICLFLEKKYVNVFLSKLPNPGNYPPSSKERRIIRRSMGVCSVVLPKERIH |
| | | SETGDLSVALDMLNELKRCPRELFDTLSPGDQERFRTISSDHNEVLQM |
| | | RSKDRFAQLVLQYIDHNRLFENLRFHVNMGKLRYLFNPKKYCIDGQTR |
| | | VRVLEHPLNGFGRLQEMEEKRLQENGPFARSGIKVRCFDEVRRDDAN |
| | | ESNYPYIVDTYTHYVLENDMVEMFFCPEGSGMKMPEVTSREGKWYV |
| | | DKKVPHCRMRMSVLELPAMLFHLLLCGAKNTEVHIGKVCDNYCHLFS |
| | | DMAQGNLTEENILSYGIKKEDIPQKVWDCVRGVHMGKDSRAYREKEI |
| | | RERYEDVTRRLERLEADRKAVLGGENKIGKRGFVQIVPGRLAAYLATDI |
| | | CRLQPSLRKGDGYGTDRLTGLNFRLLQSSIATYNCGESDILYGRFRDVF |
| | | CSAGLIGGDNPHPFLDKVLPEAYSVCCPRNTIEFYERYLEEYQRYLKPLV |
| | | IKLEKGKVPSLSFVNEGQRRWARRDDAYYHELGNLYLSQAIELPRQMF |
| | | DDEIKDKLREMPEMRDVDFDHANVTFLIGEYLKRVRHDESQEFYSWP |
| | | RHYKYVDMLKCILNPKNGSLQAVYIQMGEREGLWQERSELEEKYAKIR |
| | | LRDLGRKGLDKDEANERIKTGLGNRKKEYQKAEKVIRRYKVQDALLFM |
| | | LAKNTLFNSVEVDDERFKLKDIMPDGEKGILSEVVPMDFCFRSGNSAT |
| | | RKLMGTIHSDNTKIKNYGDFFALANDKRMVTLLPLVGEQCLVKEEVKE |
| | | EFDKYDDCRPEMISMVFDFEQWAYSAYPELKELVSNEAIKGRLFSNLL |
| | | QELLGRGELTYEEKYALVGIRNAFLHNSYPKDGGVVKVRTLPDIAKSLK |
| | | DVFKEYIRLE |
| 67 | cas13b | MKMFYKSVLTAFFTAVDSLRNKYTHYSHKDLNIREIKIECTLGGKDYCI |
| | | GLLNALDCIYDSAVNLLKLRFMAGEDEVAHLRRCKAVNKKVVVRTEKD |
| | | GFYYRLSDNGGVTEKGVIFIASMFLNRKYGFLFLKQLEGFKRSDEKRYR |
| | | LTLEAFLAFSNIKPVDRLKSDKLDRASLGLDMLNELTKIPKELSETLSVDC |
| | | LYKYLASDGEDDLRSRIRYQDRFVPLALEFISQSDEFKDFRFYTYVGNYV |
| | | YKGYIKRLIDGTDKERYLSDRLCGFYKSVNDASSDAIAQKYGVEIKDSNE |
| | | PDYMLPDSFRPHVLRATPHFVINTNNIGIKICGNDCLPIVNGKGVESPE |
| | | PDYWLSIYELPAMLFYAYLREKNGKRFKDYKSIRELIEGVEKKADEKND |
| | | RDKGALMARHIDKEIIWTQTKLDEVKRLEEKKVAAYGKKGRVVLKAGR |
| | | MADLLAHDMVRLQPATKGSDKITGANFQALQVSLAYFKRDILADVFS |
| | | RAMLTTGNHRHPFLYRIDVSHCSSLRDFYVAYLGERRKYFEDVAKKIAK |
| | | NKLNTPCHILRRLQREGSGEEAGKDVKPKFLPRGIFTDSIKNCLEQSKL |
| | | NIYIRNARNDVKPAINAAYLILMYYKEIEKGEFQGFYGEKRRYDILEEGK |
| | | PLDLAERKKALASIKPAKIDVSEANMPMSKEEHLMRKRYHAVCNNES |
| | | AIRMYQVQDILLLLMAKDIFKKALSEGVMSKKIGLENLNGIFDAPVNFV |
| | | KNFDNIKLTATGIKIKDYGKVCRLGTDFKFNSLIKAFHKVYSKSVEMDYS |
| | | DYLKEEEEFEKYRLNMVKLCREVERGITEDLHLSLDGKSHLSFNDDVIKP |
| | | YNDKYNVFNGGDLTFFINARNMFMHGDYKYECVKYVVSEHFKGSLN |
| | | DVSFAKETYGHFCNLLESMRKKTGLRIDI |
| 68 | cas12g | MLPTRYKPARTLVRPLGRLPHEPRKEFVEKCRRVRMHFEQFNIDVADL |
| | | CQWLMSLRPNTRIGDAQSTVFWDFFLNPSILTVEADEKERDRWRLAA |
| | | FDELLQIRFGHDPNAPPWSEEFRSAIRHVAQRPKSATAQRLFDRLRSL |
| | | TAPHRLVLLKSAAEWIIARYQRGMENWQRQFAEWQREKEEWEAAH |
| | | PNLTPEVRDAFTRVFKNLFENPDGDGKIGVRRKNPRICSWERLKLNKD |
| | | NCVYAGQKGHGPLCWEFSKFVKAQKNAGTIKTFFVDVANKYLHVRR |
| | | NLSKPGVKLKKSPRQEAFKRLYNQKGMEKARNWFTDAWSGYLTALN |
| | | LNEKTILDHGCLKHCGAIGAEFEKSLCQFNPHTHLCVQ.YRNALESLEPA |
| | | IRELEGDYREWRRLFLAPPRKPSFRYPSSRRLPMPKIFGEHFHQIDFDQ |
| | | SILRLRLEDMAEGEWIEFGFKPWPKDYRPGKDEVRVTSVHVNFHGNR |
| | | MRAGFHFEAPAKPSRFACTQDELDDLRSKQFPRQSQDRQLLEVARRR |
| | | LLESFDGMLESDLRILAVDLGEKGAAAAVYQGHGHEADVAIPIVKIDRL |
| | | YDHVPDVLDVESARVPPPKFDDSRDPRGVRKEHVGRHLGQLQRGAQ |
| | | TLAQHRQQDESAPAALRRHDFRSLTRHIRWMIRDWTRHNAAQITAA |
| | | AETHRCHLIVFESLRGFKPRGYDQMDFAQKARLAFFAYGRVRRKVVE |
| | | KAVERGLRVVTVPYGFTSQICSECGHRQRNKGRLRKNKYQRRFVCEC |
| | | GEPKKSANKTAAPDRSATVSPCTCRLQLGSDVNAARVLARVFWDEIV |
| | | LPTREEMREPAVDSAPPSK |
| 69 | cas12g | MCLCTLSGRTRQEEEIIGSTQYTEARSLVRRIRRPRGESRRQFKSNVLLL |
| | | RRHFEQFNVDASEICQWLMGIRPGGRHADESTGPFWEFFLDPGRFLR |
| | | ETGRGPEDADERIDAYRRIAFDVVAGIEDESRMSDPSIPRQIVESLHAV |
| | | SMATRTESARRLFERLAGLEPSHRQILLKAAAEWIVSRYWRSVQGWP |
| | | DRYKHWSDEKEEWEKAHPRLTESLREEFTGIFRDLGIRRKKPRVCPWE |
| | | RLEKGMDNCMYAGERIKVGYSRQSHSQLCAKYERFSYKQRQRTKSGK |
| | | NFKSYFVKNAELYLKLRRKNRSLIKKDVMKLFRKKVPQALWFEKAWDE |
| | | YLKALGVDEATLTKDGKLPHCTQFADDKECLFNRHTELCLQYRERLLRL |
| | | PHLQELEQLYREWRDKYLSGPRRPSLRYPSKRTLPMPKVFGRGYFCAD |
| | | FTNSLLDLRLEGMGEGDFVRFGFAPWPADYDAQPSDATVTSVHIHFV |
| | | GTRARAGFRFQAPHKTSRFASSQDEIDDLRSRKFPRAAQDGEFLDAA |
| | | RKLLLESFTGDAEREMKLLAVDLGDRGAGAAVFEGRCFKEAMPLKIIK |
| | | TDTLIDKPPPVTKTPRKGKPGKRESKRARGLDKYHVARHLDTWRKGA |
| | | RKIAERRAKGEADPVKLGAHDMRSLSLHVRWMIRDWVRLNASQIIKT |
| | | AESHKTDLIVLESLRGFSAPGYHKLDDEKKRTLAFFAYGRIRRKLTEKAV |
| | | ERGMRVVVAPYLRSSQVCAECGREQIDRNKLMKDKRKRRFICEYSDC |
| | | TWQCDSDQNAACVLGRVFWGEIELPSERKKD |
| 70 | cas12g | MHPSRYKTARTLVRRLCRLPGEDRSAFRSKVGLLRGHFEQFNVDVSEL |
| | | CQWLMSLRKRNKVPENPATFGALGDFLLQPGLPGEETDEKEADRLRL |
| | | AVFDAVAGFRMLEDRLAASIPASLSDAIRDEAVFLAGVRAAGKPSGLA |
| | | RVLARLEACAPAQRLVLLKSAAEWIVARFLRGTENWMRQRAEWEKE |
| | | KAAWEAAHPHLTPEVRAQFNKIFESLHDPENSGKPGVSRKNPRICPW |
| | | DRLKQNLDNCCYGEKGHSALCWRYQDFLKQRMGENRRDKKNFSAT |
| | | AMDLAQICREWKIQHSRNALNNPRVLDRLFAEHERRKQDKTKKESRS |
| | | PKPRQGGYKANPKADYLRSFKAHWKAYLEHMKLNDTTVLERGCLPH |
| | | CLSIKKNGKESTCKWNKHTELCLEYKRSLAPLPDSVLELEPEYREWRRL |
| | | YLHGPGRPHFRYPSAGELPLPKVFGEGFHQVDLDRSIVRLRLEGAAEG |
| | | EWLEFGFIPWPRGYQPSRREVLITSVQVHFVGTRPRAGFRFDVSHRTS |
| | | RFGCSQDELDELRSRRYPRQAQDKEFLAAARAQLIQTFEGGEGAARQ |
| | | QMRVMSVDLGEGGACASIYEGRTHQKDESLKVIKIDRRYDQHPEVLE |
| | | KDVGAAKPQKFEKSDPRGVRKEHVARHLNRIAAGASAIAEHRRKERS |
| | | DAECSVGELQEHDFRSLKRHIAWMIRDWVRLNAAQIIDVAKQHCCDL |
| | | IVFESQRGFRLPGYDELDRGKKQRFAILAFGRIRRKVVEKAVEHGMRV |
| | | VTVPYFASSQVCSACKRVQENRGSWRENKKKRVFACEFCKLKLNSDA |
| | | NASRVLARVFWGEIELPEPTRAHLPSKA |
| 71 | cas12g | MPVSRYSESRTLVRPLARLPHEERQDVTPKVARLRRHFERFNVDVAEL |
| | | CQWLMGLRNQFGPKESPASFGPLGDFLIEPALDNIDADETERDRWRL |
| | | AVFDAVAGFRPIRGLGDHPVPDTLRLAMQQAASLSPTPTTARLLERLR |
| | | PLSPAHRLVLLKSAAEWIVARYQRGMENWVIQHAAWHKEKEAWER |
| | | EHPALTPAVRERFTALYKQLSDSKPTDRPVSRRKNPRICEWERLRQNID |
| | | NCCYAGEKGHGPLCRKYANFVKARKAVDGKFNDLLFWDTATSFIALC |
| | | RKFNVTRARNALQSQLDALFAEDQRRKAERDQAKGRQPRPLHPQAA |
| | | ARAKSDFLRIFKDGWNAYLSAMGLNDSTAIEKGRLPHCQKIGGTFENS |
| | | KCEWNPHTDLCHQYRRLAGQLDDATLALEKDYREWRRLYLAGPRKPS |
| | | FQYPSSRDLPMPKIFGAGFFELDMDRSILRLRLDDMVEGEWLEFGFKP |
| | | WPREYTPSRAQVARPGRITSVHVNFIGSRCRVGFRFEAPHAGSRFGCS |
| | | QDEIDQLRRDHPRERDDQPFLEAARKRLVETFAGDARRDLRLLAVDV |
| | | GEKGCCAAVYQGTRYVADALLPIIKINQLYTEPPTELKPDSHNRPAPDR |
| | | RPFNDEKDPRDPRGVRKEHVARHLKRMADKAPEVAAYRLAQREKAA |
| | | PSPSASPPPVTLGVHDFRRLKRHVTWMIRDWARHNAARIVAEAQRH |
| | | GCDLIVFESHRGRRPPGYHEVGDDAERRKLDNATFAFGRIRRKVTEKA |
| | | VERGLRVVTVPYHCSSKVCSRCGRLQENDGLLRRNKKERKFICEQCKF |
| | | ETNSDGNAARVLARVFWGEIMLPSPEERRKKREGSGGRSPTPANPG |
| | | GLVDAPPSRRNLR |
| 72 | Cas12 | MEDYSGFVNIYSIQKTLRFELKPVGKTLEHIEKKGFLK |
| | | KDKIRAEDYKAVKKIIDKYHRAYIEEVFDSVLHQKKK |
| | | KDKTRFSTQFIKEIKEFSELYYKTEKNIPDKERLEALSE |
| | | KLRKMLVGAFKGEFSEEVAEKYKNLFSKELIRNEIEKF |
| | | CETDEERKQVSNFKSFTTYFTGFHSNRQNIYSDEKKST |
| | | AIGYRIIHQNLPKFLDNLKIIESIQRRFKDFPWSDLKKN |
| | | LKKIDKNIKLTEYFSIDGFVNVLNQKGIDAYNTILGGK |
| | | SEESGEKIQGLNEYINLYRQKNNIDRKNLPNVKILFKQI |
| | | LGDRETKSFIPEAFPDDQSVLNSITEFAKYLKLDKKKK |
| | | SIIAELKKFLSSFNRYELDGIYLANDNSLASISTFLFDD |
| | | WSFIKKSVSFKYDESVGDPKKKIKSPLKYEKEKEKWL |
| | | KQKYYTISFLNDAIESYSKSQDEKRVKIRLEAYFAEFK |
| | | SKDDAKKQFDLLERIEEAYAIVEPLLGAEYPRDRNLK |
| | | ADKKEVGKIKDFLDSIKSLQFFLKPLLSAEIFDEKDLGF |
| | | YNQLEGYYEEIDSIGHLYNKVRNYLTGKIYSKEKFKL |
| | | NFENSTLLKGWDENREVANLCVIFREDQKYYLGVMD |
| | | KENNTILSDIPKVKPNELFYEKMVYKLIPTPHMQLPRII |
| | | FSSDNLSIYNPSKSILKIREAKSFKEGKNFKLKDCHKFI |
| | | DFYKESISKNEDWSRFDFKFSKTSSYENISEFYREVERQ |
| | | GYNLDFKKVSKFYIDSLVEDGKLYLFQIYNKDFSIFSK |
| | | GKPNLHTIYFRSLFSKENLKDVCLKLNGEAEMFFRKK |
| | | SINYDEKKKREGHHPELFEKLKYPILKDKRYSEDKFQF |
| | | HLPISLNFKSKERLNFNLKVNEFLKRNKDINIIGIDRGE |
| | | RNLLYLVMINQKGEILKQTLLDSMQSGKGRPEINYKE |
| | | KLQEKEIERDKARKSWGTVENIKELKEGYLSIVIHQIS |
| | | KLMVENNAIVVLEDLNIGFKRGRQKVERQVYQKFEK |
| | | MLIDKLNFLVFKENKPTEPGGVLKAYQLTDEFQSFEK |
| | | LSKQTGFLFYVPSWNTSKIDPRTGFIDFLHPAYENIEKA |
| | | KQWINKFDSIRFNSKMDWFEFTADTRKFSENLMLGKN |
| | | RVWVICTTNVERYFTSKTANSSIQYNSIQITEKLKELFV |
| | | DIPFSNGQDLKPEILRKNDAVFFKSLLFYIKTTLSLRQN |
| | | NGKKGEEEKDFILSPVVDSKGRFFNSLEASDDEPKDA |
| | | DANGAYHIALKGLMNLLVLNETKEENLSRPKWKIKN |
| | | KDWLEFVWERNR |
| 73 | Cas12 | MKKFTNLYSLSKTLRFELIPQGKTLENIQKSGILEQDNSRAEKYEKIKKI |
| | | IDDYHKFFIEKSFTGKKIDDYFLNQYFELFKIKDKDEEQKKDFKSIQEN |
| | | LRKNIISFFDKNKLKRLFEKEIIKEDLPNFVKEEEDKKLISEFDKFTTYFV |
| | | GFHENRKSMYSEEEKSTSIAYRTINENLPKFINNIFVFEKISKTPISENF |
| | | RELYKDLEEYLNVNDIQDIFKLNYFSNVITQKQIDVYNLVIGGKTLEN |
| | | GTKIKGLNEYINLYNQNQTDKKNKLPLLTVLFKQILCDRDTISFLPEQF |
| | | ENDIDVLDNIKNTYSNMEKSIKDIKDLLSNLKDFDLSKIYITNDIALTDI |
| | | SQQVFNNYSIIINAIKENIKKENPKKKTENEEKYGERIDKIFKSNNSFSI |
| | | KYINDCIKEKNIEIYFMDFGKKENNKKVKNLFDELQNNYSMVKDLLE |
| | | YKKIQSLIQDEKSIELIKNFLDSIKNIQHFLKPLYVKDNDIVKDISFYRDF |
| | | EELYLNIDKITPLYNKVRNYVTQKPYSVKKIKLNFENSTLLAGWDLNK |
| | | ERDNTCAILRKDDLYYLAIMDVNNRNVFNEKGIDGIGYEKMEYKLLP |
| | | GANKMLPKVFFSKSRIKDFNPSEQIIRNYEKETHKKGSNFSLKDCHKL |
| | | IDFFKSSINKHEDWKNFNFKFSNTDKYEDLSGFYREVEQQGYKITFR |
| | | NISKEYVDKLVEEGKIYLFQIYNKDFSKYSKGTPNMHTLYWKALFDE |
| | | DNLKNVVYKLNGQAEIFYRKGSIEKENIVIHKANNAIENKNMDNKKK |
| | | QSKFEYDIIKDRRYTVDKFQFHVPITLNFKAIGNERINEQVNQYIKDN |
| | | NIKHIIGIDRGERHLLFLSLIDLKGNIIKQFSLNEIVNEYNGNSYKTNYH |
| | | MLLEKREEERDKARKSWKTIENIKELKEGYISQVIHKITQLMIEYNAIV |
| | | VLEDLNFGFMRGRQKVEKQVYQKFEKMLIDKLNYLVDKKKDKNEA |
| | | GGLLKAHQLTNKFESFQKMGKQNGFLFYIPAWNTSKLDPITGFVNL |
| | | FDTHYTNVDNAKKFFENFEDIRFNEKKNYFEFIVNDYTKFNTKAEGT |
| | | KLNWTICSNEDRIKTFRSSSKNNQWVSETVNLTDSLIELFKKYDIDYK |
| | | LELKEQIISKSEKNFFETLLYLFKLTLQMRNSITGTETDYLISPVADKTG |
| | | NFFDSRKGIENLPNNADANGAYNIARKGLWVIEQIKKAKDLKKVKLA |
| | | ISNKEWLQFVQGK |
| 74 | Cas12 | MAKNTIFSQFTGLYPVSKTLRFELKPMGKTLEKIKETGVIENDKKRHN |
| | | DYFDAKKIIDKYHKYFIDAALSKFPRIDWSPLKEAIERSLDRSDASKKKL |
| | | EKTQTEFRKKIAKALTTHDHYKELTASTPKDLFLKVFPDHFGKQPAID |
| | | TFDGFSSYFTGFQENRQNIYSDEAISTAIPYRLVHDNFPKFLSNIEVYK |
| | | TLKDNAPSVLSDAENELRDFLNGKSLANIFELNAYNEVLTQSGIDFFN |
| | | QVIGGISDEGGEKKTRGINEFSNLYRQQHPEFAQKRLATKMIPLYKQ |
| | | ILSDRETKSFILESYSNDSQVQNSVKEFFESQILNWDIAGRRVNVLNE |
| | | LTSLVKRISEFDLGNIYVNQEELSNISLKLFDNWNSINGLLFKHAENRI |
| | | GSAEKSANKKKIDAWMKNKEFSIATLNLAIAESNSEEISRVKIESYWN |
| | | NFEAKVQSILCGDNRRNLDEFISATFNENNALREDSKIIEKLKAFLDAL |
| | | lEIMHSIKPLISDAENRDLSFYNELIPLYDQLSLVVPLYNKIRNYATQKL |
| | | TESEKFKLNFDNPTLADGWDQNKEEANTAILLLKNGLYYLGIMNAK |
| | | NKPKIKDFKTSESEDCYDKMVYKLLPGPNKMLPKVFFSEKGLATFKP |
| | | PKDILDGYNAGKHKKGDLFDIGFCHQLIDFFKESIAKHPDWKKFDFN |
| | | FSDTSSYEDISGFYKEVTDQGYKITFSKIPTSQIDEWVKEGKLFLFQIY |
| | | NKDFAPGAKGSPNLHTLYWKSVFSPENLKDVVVKLNGEAELFYRPS |
| | | SVKKPYSHKVGEKLVNRIGKDGLPLPESVFGELFRYFNGKLEGELSDE |
| | | AKRYLDVAVVKDVKHEIVKDRRYTQDKFEFHVPLTLNFKADSKNEY |
| | | MNERVRHFLKDNPDVNIIGIDRGERHLLYMTLINQKGEILKQKSFNV |
| | | VESVNYQAKLVQREKERDAARRSWSSVGKIKDLKEGFLSQVIHEITT |
| | | TMIENNAIVVLEDLNFGFKRGRFCVERQVYQKFEKMLIDKLNYLVFK |
| | | NKPEGDVGGVLKGYQLAEKFDSFQKLGKQSGFLFYIPAAYTSKIDPT |
| | | TGFANLFNMTELTSAEKKKEFLSHFEDITYDGKNDRFLFSFDYKNFKC |
| | | FQTDYIKKWTVYSQGKRIVYDKESKSAKEISPVEIIKAALAKQNIALTD |
| | | QLDVLSAINSVEASPKSASFFGDICYAFEKTLQMRNSIPNTDEDYLAS |
| | | PVMNKRGEFYDSRSCDDALPQNADANGAYHIALKGLYLIKNVFDA |
| | | GGKELKISHEDWFKFAQSRNC |
| 75 | Cas12 | MSKGKIWENFINQYSVSKTLRFELKPVGKTLENINAKGLIEEDEQRAE |
| | | DYKKAKKIIDEYHKYFIEGALGSCSLDLNILNEFLQLYNKAQKTDADKK |
| | | EYEKIQTTLRKNIAESFGKNADKKTKEQYENLFKKELLRNDLPDWVE |
| | | DEEDAKIIERFKTFTTYFTGFHENRKNIYDNEEKSTAIGYRIVHENLPK |
| | | FIDNMNAFEKISKALDLSEIDRDFQSELGEIKAEEFFTIEFFNQCLNQF |
| | | GIDRYNTLLGGISEGENIKKKQGLNERINLYNQQLKGERKKERLPKLK |
| | | VLYKQILSDSSSHSFSIDEFENDNELLESLEIFYKNELIGFNHSGVDSNI |
| | | FDLVKDLLLKIDESEQSSIYLKNDKGLTEISQRIFGDWNIIKSALEEYYD |
| | | EHYPPKKDTFNKKELDERSRWLKENHSIGVIEKALANYENEIVREHLK |
| | | QNSAPIVSYFKSLEVDGENLIDKIYSAYGNISDLLNSSYPDEKKLVSDR |
| | | TSKDKIKVFLDSLMSLLHFLKPLDVKDLGNKDSAFYGDYDFIVEQLSK |
| | | LVRLYNKTRNYLTRKPYSIEKIKLNFENSTLLAGWDVNKERDNNCVIF |
| | | KRQDGDRELFYLGIMDKSHNKIFTKIEEAKSDDVYQKMNYKLLPGP |
| | | NKMLPKVFFSKKSIDFYAPGEELLKNYKNGTHKKGENFNLQHCHELI |
| | | DFFKRSINKHEDWSQFNFKFSDTSEYEDTSFFFKEVSQQGYSITFKNI |
| | | DRETIEKFVDEGKLYLFQIYNKDFSPKSKGRPNLHTLYWKMLFDERN |
| | | LANTVYQLNGEAEVFYRKKSISEKDRVVHRADEPIGLKNSENSAQKS |
| | | LFPYDIVKDRRFTVDKFQFHVPITLNFKSEGNERLNISVNKFLKDNPD |
| | | VNIIGLDRGERHLIYLTLINQKGEILHQESLNEVMGVNYQQKLHRVE |
| | | KDRTEERRNWDRIENIKELKSGYLSQVVHKISQLMVEYNAIVVMEDL |
| | | NFGFKRGRIKVEKQVYQKFEKTLIDKLNYLVFKDREPEEPAGVLNAL |
| | | QLTNKFESFKKLGKQCGFLFYVTSDYTSKIDPATGFVNLLYPKYESVEK |
| | | SQNFFRKFDNICFNSGAGYFEFDFDYSNFTDRADGTRTRWKVCTVG |
| | | NERFGYNPKTKASETVNVTESLKELLLQHEIAFENGESLVESISKNTTK |
| | | YFHKSLLNFLRLTLTLRHSKTGTDIDYILSPVANEEGVFFDSRNASDK |
| | | MPKDADANGAYNVALKGLMVLERINAAEDLSQFKFKDMSIKNKD |
| | | WLKFVQDRQG |
| 76 | Cas12 | MKNLANFTNLYSLQKTLRFELKPIGKTLDWIIKKDLLKQDEILAEDYKI |
| | | VKKIIDRYHKDFIDLAFESAYLQKKSSDSFTAIMEASIQSYSELYFIKEKS |
| | | DRDKKAMEEISGIMRKEIVECFTGKYSEVVKKKFGNLFKKELIKEDLL |
| | | NFCEPDELPIIQKFADFTTYFTGFHENRENMYSNEEKATAIANRLIRE |
| | | NLPRYLDNLRIIRSIQGRYKDFGWKDLESNLKRIDKNLQYSDFLTENG |
| | | FVYTFSQKGIDRYNLILGGQSVESGEKIQGLNELINLYRQKNQLDRR |
| | | QLPNLKELYKQILSDRTRHSFVPEKFSSDKALLRSLLDFHKEVIQNKNL |
| | | FEEKQVSLLQAIRETLTDLKSFDLDRIYLTNDTSLTQISNFVFGDWSKV |
| | | KTILAIYFDENIANPKDRQRQSNSYLKAKENWLKKNYYSIHELNEAIS |
| | | VYGKHSDEELPNTKIEDYFSGLQTKDETKKPIDVLDAIVSKYADLESLL |
| | | TKEYPEDKNLKSDKGSIEKIKNYLDSIKLLQNFLKPLKPKKVQDEKDLG |
| | | FYNDLELYLESLESANSLYNKVRNYLTGKEYSDEKIKLNFKNSTLLDG |
| | | WDENKETSNLSVIFRDTNNYYLGILDKQNNRIFESIPEIQSGEETIQK |
| | | MVYKLLPGANNMLPKVFFSEKGLLKFNPSDEITSLYSEGRFKKGDKF |
| | | SINSLHTLIDFYKKSLAVHEDWSVFNFKFDETSHYEDISQFYRQVESQ |
| | | GYKITFKPISKKYIDTLVEDGKLYLFQIYNKDFSQNKKGGGKPNLHTIY |
| | | FKSLFEKENLKDVIVKLNGQAEVFFRKKSIHYDENITRYGHHSELLKG |
| | | RFSYPILKDKRFTEDKFQFHFPITLNFKSGEIKQFNARVNSYLKHNKD |
| | | VKIIGIDRGERHLLYLSLIDQDGKILRQESLNLIKNDQNFKAINYQEKL |
| | | HKKEIERDQARKSWGSIENIKELKEGYLSQVVHTISKLMVEHNAIVVL |
| | | EDLNFGFKRGRQKVERQVYQKFEKMLIEKLNFLVFKDKEMDEPGGI |
| | | LKAYQLTDNFVSFEKMGKQTGFVFYVPAWNTSKIDPKTGFVNFLHL |
| | | NYENVNQAKELIGKFDQIRYNQDRDWFEFQVTTDQFFTKENAPDT |
| | | RTWIICSTPTKRFYSKRTVNGSVSTIEIDVNQKLKELFNDCNYQDGED |
| | | LVDRILEKDSKDFFSKLIAYLRILTSLRQNNGEQGFEERDFILSPVVGS |
| | | DGKFFNSLDASSQEPKDADANGAYHIALKGLMNLHVINETDDESLG |
| | | KPSWKISNKDWLNFVWQRPSLKA |
| 77 | Cas12 | MNLIENETKSEEIKSKLDSIMEIMHWTKMFIIEEEIEKDVNFYNEIEEI |
| | | YDELQPLVTIYNRIRNYVTQKPYSEEKIKLNFGIPTLANGWSKTKEYD |
| | | NNAIIMIRDGKYYLGIFNAKNKPDKKIMEGHQSEENGDYKKMIYRLL |
| | | PGPNKMLPKVFMSKTGIAEYKPSQYILECYEQNKHIKSDKNFDIKFCR |
| | | DLIDFFKTSINRHPEWSKFNFKFSETSEYEDISTFYREVEKQGYKIEWT |
| | | YISEKEIKELDENGQLYLFQIYNKDFSEKSKGKENLHTMYLKNLFSEEN |
| | | LKNIVLKLNGEAEVFFRKSSIKKPIIHKKGSVLVNKTYNENGERKSIPEE |
| | | QYTEIYKYLNSIGTNELSEKSKKLMEEGKVEYYKANYDIVKDYRYSVD |
| | | KFFIHLPMTINFKAAGFSPINNIALKSIALKEDMHIIGIDRGERNLIYVS |
| | | VIDTKGNIVEQRNFNIVNGIDYKEKLKQKELDRDNARKNWKEIGKIK |
| | | DLKEGYLSLVVHEIAKLVVKYNAIITMEDLNQGFKRGRFKVERQVYQ |
| | | KFETMLINKLNYLVDKDLAVDQEGGLLRGYQLTYIPESLKVLGRQCG |
| | | YIFYVPVAYTSKIDPTTGFVAIFNYKGMTDKDFVTSFDSIKYDDERGLF |
| | | AFEFDYENFVTHKVEMARNKWTVYTYGERIKRKFKNGLWDTAEKV |
| | | DLTYQMRSILEKYEIEYNKGQDILEQIEELDEKAQNGICKEIKYLVKDI |
| | | VQMRNSLPDNAVEDYDAIISPVINNNGEFFDSTRGDEDKPLDADAN |
| | | GAYCIALKGLYEVMQIKKNWNEETEFPRKELKIRHQDWFDFIQNKR |
| | | YL |
| 78 | Cas12 | MENRYQVLQGLTAAQKKAAAAAKKRSSFSIVELNAATRSRVPDEKY |
| | | VPVQNYFSAMGKVCSQGEPKRENFVTRICAAYQELEEYIPSIRKSLLQ |
| | | EKRATELIKNYLDAVNDLLRFIKPLLGRGNETDKDANFYGEFSFLTDC |
| | | LFAIVPLYNEVRNYLTQKPYSTEKFKLNFRGSTLLNGWDKNKERDNL |
| | | GVILRKEGKYFLAIMNKKHNTLFTEGKLQQHTGGECYQKMEYKLIPG |
| | | SKMLPKVFFSKKGISTFQPSEELLLNYRIGTYKKGEKFNLEHLHKLIDFY |
| | | KHSIAVHEDWSKFDFHFSDTSSYRDISGFYKEVEQQGYKLTFRNVSV |
| | | SYINRLVEEGKLYLFQIYNKDFSEYSKGTPNLHTLYWKMLFDPENLKD |
| | | VVYKLSGEAEVFFRKKSLDVSHPTHPKNEPIEKKNINNKGEKSLFSYD |
| | | LIKDRRFTVDKFQFHVPITMNFKGEQGDRVNQMVQSYVRNNKGL |
| | | NVIGIDRGERNLLYLVVINEHGEILEQFSLNEIRNAYNGKEHKIDYHTL |
| | | LEERSKKRQDARQSWQTIEGIKDLKTGYLSQVIHVITQLMVKYNAIV |
| | | VLEDLNFGFKSSRQKFEQSVYQQFERKLIDKLNFLVNKKAAPNEVGG |
| | | LLNAYQLTAPLGNSRKMGKQNGFLFYVPAWHTSKIDPRTGFVNLLD |
| | | TRYENVAKAKEFFAKFASITYNPEKKWFEFAFDYKAFGNRADGSRTK |
| | | WTICSYGERIETFRNPENNNQWDTKSVPLTERLTELFSKYGIDYTTNL |
| | | KEQILNQTDKAFFVELLGALRLTLQLRNSRKSTGEDFLFSPVADENGC |
| | | FFDSREANDNEPKDADANGAYHIALKGLWVLDTIRNTEEGKNPKLA |
| | | ITNKEWLSFAQAKPFAHE |
| 79 | Cas12 | MSDSYDELTKAQKEKQEKRKHVALTEVVAALEKYTIALDNGHEHKN |
| | | AVNTFKNYFQNYFFHFDTDKKKTAKTLDCQIKDEYNGLKGILNTPW |
| | | DKNKKLQQDKKLVQQIKSFLDSIQELLWFIKPLVLTDNTLEKDERFYG |
| | | EFMPLYDEISNIIKLYNKIRNYLTKKPYSIEKYKLNFENGSLLSGWDVN |
| | | KEKDNTSVLLCKDNQYYLAIMHIDHNKVFELDELIKHAGKGYQKINY |
| | | KLLPGANKMLPKVFFSGKNISYYDPSKEILKIRNYGTHTKNGDPQPG |
| | | FSKRDFSVDDCRKMIDFFKNSIAKHEDWKNFDFKFQPTKNYNSIDEF |
| | | YREVEEQGYKITYSNVSEDYIDSLVEYGKIYLFHIYNKDFSDKRDESKK |
| | | HTDNMHTLYWKALFDAKNLKDVVYKLNGEAEIFYRKKSIDIKKPTHE |
| | | KGKPIDNKNPNARKKTSVFKYDLIKDKRFTVDKFFFHVPITLNFKSKS |
| | | GYLSNDDVNAAIKKNNDIKIIGLDRGERNLIYLSLINSKGEIAYQESLN |
| | | VVSTDKGFDVNYHKLLDDKEGNRDEARKNWDKIENIKELKAGYLSQ |
| | | VIHKIAKLMIDNNAIVVMEDLNFGFKRGRFKVEKQIYQKFEKMLIDK |
| | | LNYLVFKNVHPEQAGGLYKAYQLTAQFESFKKLGKQSGFLFYIPAW |
| | | NTSKIDPTAGFVDFLKPRYESVTQAKSFLQRFDKINYNKTKDYFEFAF |
| | | DYKNFTDKANDTKTDWVVCTYGTERYYYDVRTKTTQKIDITAELKKL |
| | | LEKSEINYLNGKDIKELIIAVDSKEFHSALLKYLAIVLALRYSDSQSGRD |
| | | FILSPVANEQGHFFNSDKTDDTLPKDADANGAYHIALKGLWAINQI |
| | | RKTKNGDKLKLTISNKDWLNFVQKKEYRKGV |
| 80 | Cas12 | MQTLFENFTNQYPVSKTLRFELIPQGKTKDFIEQKGLLKKDEDRAEK |
| | | YKKVKNIIDEYHKDFIEKSLNGLKLDGLEKYKTLYLKQEKDDKDKKAFD |
| | | KEKENLRKQIANAFRNNEKFKTLFAKELIKNDLMSFACEEDKKNVKE |
| | | FEAFTTYFTGFHQNRANMYVADEKRTAIASRLIHENLPKFIDNIKIFEK |
| | | MKKEAPELLSPFNQTLKDMKDVIKGTTLEEIFSLDYFNKTLTQSGIDIY |
| | | NSVIGGRTPEEGKTKIKGLNEYINTDFNQKQTDKKKRQPKFKQLYKQ |
| | | ILSDRQSLSFIAEAFKNDAEILEAIEKFYVNELLHFSNEGKSTNVLDAIK |
| | | NAVSNLESFNLTKMYFRSGTSLTDVSRKVFGEWSIINRALDNYYATT |
| | | YPIKPREKSEKYEERKEKWLKQDFNVRLIQTAIDEYDNETVKGKNSG |
| | | KVIADYFAKFCDDKETDLIQKVNEGYIAVKDLLNTPYPENEKIGSNKD |
| | | QVKQIKAFMDSIMDIMHFVRPLSLKDTDKEKDETFYSLFTPLYDHLT |
| | | QTIALYNKVRNYLTQKPYSTEKIKLNFENSTLLGGWDLNKETDNTAII |
| | | LRKDNLYYLGIMDKRHNRIFRNVPKADKKDFCYEKMVYKLLPGANK |
| | | MLPKVFFSQSRIQEFTPSAKLLENYANETHKKGDNFNLNHCHKLIDF |
| | | FKDSINKHEDWKNFDFRFSATSTYADLSGFYHEVEHQGYKISFQSIA |
| | | DSFIDDLVNEGKLYLFQIYNKDFSPFSKGKPNLHTLYWKMLFDENNL |
| | | KDVVYKLNGEAEVFYRKKSIAEKNTTIHKANESIINKNPDNPKATSTF |
| | | NYDIVKDKRYTIDKFQFHIPITMNFKAEGIFNMNQRVNQFLKANPDI |
| | | NIIGIDRGERHLLYYALINQKGKILKQDTLNVIANEKQKVDYHNLLDK |
| | | KEGDRATARQEWGVIETIKELKEGYLSQVIHKLTDLMIENNAIIVME |
| | | DLNFGFKRGRQKVEKQVYQKFEKMLIDKLNYLVDKNKKANELGGLL |
| | | NAFQLANKFESFQKMGKQNGFIFYVPAWNTSKTDPATGFIDFLKPR |
| | | YENLNQAKDFFEKFDSIRLNSKADYFEFAFNFKNFTEKADGGRTKWT |
| | | VCTTNEDRYAWNRALNNNRGSQEKYDITAELKSLFDGKVDYKSGK |
| | | DLKQQIASQESADFFKALMKNLSITLSLRHNNGEKGDNEQDYILSPV |
| | | ADSKGRFFDSRKADDDMPKNADANGAYHIALKGLWCLEQISKTDD |
| | | LKKVKLAISNKEWLEFVQTLKG |
| 81 | Cas12 | MQLTDNLSDKYKEAAPLLNENYSNEKGLKNDDKSISLIKNFLDAIKEI |
| | | EKFIKPLSETNITGEKNDLFYSQFTPLLDNISRIDILYDKVRNYVTQKPF |
| | | STDKIKLNFGNSQLLNGWDRNKEKDCGAVWLCKDEKYYLAIIDKSN |
| | | NSILENIDFQDCDESDCYEKIIYKLLPGPNKMLPKVFFSEKCKKLLSPS |
| | | DEILKIRKNGTFKKGDKFSLDDCHKLIDFYKESFKKYPNWLIYNFKFKK |
| | | TNEYNDIREFYNDVASQGYNISKMKIPTSFIDKLVDEGKIYLFQLYNK |
| | | DFSPHSKGTPNLHTLYFKMLFDERNLEDVVYKLNGEAEMFYRPASIK |
| | | YDKPTHPKNTPIKNKNTLNDKKTSAFPYDLIKDKRYTKWQFSLHFPIT |
| | | MNFKAPDRAMINDDVRNLLKSCNNNFIIGIDRGERNLLYVSVIDSN |
| | | GTIIYQHSLNIIGNKFKGKTYKTNYREKLATREKDRTEQRRNWKAIESI |
| | | KELKEGYISQAVHVICQLVVKYDAIIVMEKLTEGFKRGRTKFEKQVYQ |
| | | KFEKMLIDKLNYYVDKKLDPDEEGGLLHAYQLTNKLESFDKLGTQSG |
| | | FIFYVRPDFTSKIDPVTGFVNLLYPRYENIDKAKDMISRFDEIRYNAGE |
| | | DFFEFDIDYDKFPKTASDYRKKWTICTNGERIEAFRNPANNNEWSY |
| | | RTIILAEKFKELFDNNSINYRDSDDLKAEILSQTKGKFFEDFFKLLRLTL |
| | | QMRNSNPETGEDRILSPVKDKNGNFYDSSKYDEKSKLPCDADANG |
| | | AYNIARKGLWIVEQFKKADNVSTVEPVIHNDKWLKFVQENDMTNN |
| 82 | Cas12 | MLPNEKERNEFKNSNAKQYIREISNIITDTETAHLEYDEHISLIESEEKA |
| | | DEMKKRLDMYMNMYHWAKAFIVDEVLDRDEMFYSDIDDIYNILE |
| | | NIVPLYNRVRNYVTQKPYNSKKIKLNFQSPTLANGWSQSKEFDNNA |
| | | IILIRDNKYYLAIFNAKNKPDKKIIQGNSDKKNDNDYKKMVYNLLPGA |
| | | NKMLPKVFLSKKGIETFKPSDYIISGYNAHKHIKTSENFDISFCRDLIDY |
| | | FKNSIEKHAEWRKYEFKFSATDSYNDISEFYREVEMQGYRIDWTYISE |
| | | ADINKLDEEGKIYLFQIYNKDFAENSTGKENLHTMYFKNIFSEENLKDI |
| | | IIKLNGQAELFYRRASVKNPVKHKKDSVLVNKTYKNQLDNGDVVRIP |
| | | IPDDIYNEIYKMYNGYIKENDLSEAAKEYLDKVEVRTAQKDIVKDYRY |
| | | TVDKYFIHTPITINYKVTARNNVNDMAVKYIAQNDDIHVIGIDRGER |
| | | NLIYISVIDSHGNIVKQKSYNILNNYDYKKKLVEKEKTREYARKNWKSI |
| | | GNIKELKEGYISGVVHEIAMLMVEYNAIIAMEDLNYGFKRGRFKVER |
| | | QVYQKFESMLINKLNYFASKGKSVDEPGGLLKGYQLTYVPDNIKNLG |
| | | KQCGVIFYVPAAFTSKIDPSTGFISAFNFKSISTNASRKQFFMQFDEIR |
| | | YCAEKDMFSFGFDYNNFDTYNITMSKTQWTVYTNGERLQSEFNNA |
| | | RRTGKTKSINLTETIKLLLEDNEINYADGHDVRIDMEKMDEDKNSEF |
| | | FAQLLSLYKLTVQMRNSYTEAEEQEKGISYDKIISPVINDEGEFFDSD |
| | | NYKESDDKECKMPKDADANGAYCIALKGLYEVLKIKSEWTEDGFDR |
| | | NCLKLPHAEWLDFIQNKRYE |
| 83 | Cas12 | MSNLYSNLHNLYPVQKTLRFELKPQGKTKENMEKAGILKADEHRAE |
| | | VYGKVKKYCDEYHKTFIDRCLSNIELNEIDKYYELYSINNRDDKQKEEL |
| | | DQLETGLRKQISDAFKKSAEYKGLFQKDMITSYLVTMYKENQEKMQ |
| | | DIGEFNRFTTYFTGYNKNRENMYSEEDKSTAISYRLINENLPTFIDNIK |
| | | IYKKIVSLMPENIEKIYKDLEEYIQVNSVDEIFNISYYNDVLTQRGIECY |
| | | NILISGRTKNDGDKIKGLNEYINEFNQTHNEKIPKLQELYKQILSDAES |
| | | ASFKVDIIENDKELLNLIEVYYANILPTLNKIEDLFTRISNYNLELILVNN |
| | | DGSLSTLSNMVFNEWSYIKGIISQKYDAEYSGKEKYGTEKYAQKKQE |
| | | YLKKQKIYSLKFLNDCIGNNAICEYLKNYIIQNKNIETIKEDYNEVQNIK |
| | | AEDDTKELIKDEKSIEKIKKFLDDVKSLQEFVKLVIPKDRTVEKDAKFYS |
| | | ELTPYYEKIKEIIPLYNKVRNYVTQKPYSTEKIKLNFECPTLLNGWDAN |
| | | KEEANLGVILLKEGKYYLGIMNPYCKKIFEVYEKDSNEQNNYKKMEY |
| | | KLLPGPNKMLPKVFFSNSRIEEFNPSKELQEKYNKGYHKKGKDFDIN |
| | | FCHELIDFYKQSLNKHEDWKKFNFKFKDTSEYNDISEFYREVEEQGY |
| | | KIEYTEYSEKYINELVDRGELYLFQIYNKDFSEYSKGKENLHTLYWKAV |
| | | FDPDNIMNPVYKLNGNAEIFYRKKSLEMKVTHPANQPIANKNISTIE |
| | | AGRSTSTFKYDLIKDKRYTMDKFQFHVPITVNFKSERLFNINQIVNKY |
| | | LKYNDDIHVIGIDRGERNLLYVCVIDKNEKIVYQKSLNEIVSEYNNNR |
| | | YTTDYHGLLDRKEKEREIAREDWKNIENIKELKEGYMSQIIHILVELM |
| | | KKYNAIIVIEDLNKGFKNSRIKVEKQVYQKFEKMFIDKLNYLVFKDED |
| | | KMDEGGVLNAYQLTNKFESFTKLGKQSGILYYIPAWCTSKIDPTTGFI |
| | | NRFYLKYENFDKSKEFVNRIDDIRYNEKENLFEFDIDYSKFTDRLNDTK |
| | | NKWTLCSYGERILTQKNANGEWFDRRIQLSIEFKNLFEKYVINLNNIK |
| | | DSILKLDKDNIEFYKGNGENLGFIQLFKLMVQMRNSLTGKEEDNLIS |
| | | PVKNQHGKFFNTSERVEGLPIDADANGAYNIARKGFMLVEQMKNV |
| | | EDEKLNKIKYNITEKEWLNYVQNRGMWWKRQYLYHI |
| 84 | Cas12 | MAKNTIFSQFTGLYPVSKTLRFELKPMGKTLEKIKETGVIENDKKRHN |
| | | DYFDAKKIIDKYHKYFIDAALSKFPRIDWSPLKEAIERSLDRSDASKKKL |
| | | EKTQTEFRKKIAKALTTHDHYKELTASTPKDLFLKVFPDHFGKQPAID |
| | | TFDGFSSYFTGFQENRQNIYSDEAISTAIPYRLVHDNFPKFLSNIEVYK |
| | | TLKDNAPSVLSDAENELRDFLNGKSLANIFELNAYNEVLTQSGIDFFN |
| | | QVIGGISDEGGEKKTRGINEFSNLYRQQHPEFAQKRLATKMIPLYKQ |
| | | ILSDRETKSFILESYSNDSQVQNSVKEFFESQILNWDIAGRRVNVLNE |
| | | LTSLVKRISEFDLGNIYVNQEELSNISLKLFDNWNSINGLLFKHAENRI |
| | | GSAEKSANKKKIDAWMKNKEFSIATLNLAIAESNSEEISRVKIESYWN |
| | | NFEAKVQSILCGDNRRNLDEFISATFNENNALREDSKIIEKLKAFLDAL |
| | | lEIMHSIKPLISDAENRDLSFYNELIPLYDQLSLVVPLYNKIRNYATQKL |
| | | TESEKFKLNFDNPTLADGWDQNKEEANTAILLLKNGLYYLGIMNAK |
| | | NKPKIKDFKTSESEDCYDKMVYKLLPGPNKMLPKVFFSEKGLATFKP |
| | | PKDILDGYNAGKHKKGDLFDIGFCHQLIDFFKESIAKHPDWKKFDFN |
| | | FSDTSSYEDISGFYKEVTDQGYKITFSKIPTSQIDEWVKEGKLFLFQIY |
| | | NKDFAPGAKGSPNLHTLYWKSVFSPENLKDVVVKLNGEAELFYRPS |
| | | SVKKPYSHKVGEKLVNRIGKDGLPLPESVFGELFRYFNGKLEGELSDE |
| | | AKRYLDVAVVKDVKHEIVKDRRYTQDKFEFHVPLTLNFKADSKNEY |
| | | MNERVRHFLKDNPDVNIIGIDRGERHLLYMTLINQKGEILKQKSFNV |
| | | VESVNYQAKLVQREKERDAARRSWSSVGKIKDLKEGFLSQVIHEITT |
| | | TMIENNAIVVLEDLNFGFKRGRFCVERQVYQKFEKMLIDKLNYLVFK |
| | | NKPEGDVGGVLKGYQLAEKFDSFQKLGKQSGFLFYIPAAYTSKIDPT |
| | | TGFANLFNMTELTSAEKKKEFLSHFEDITYDGKNDRFLFSFDYKNFKC |
| | | FQTDYIKKWTVYSQGKRIVYDKESKSAKEISPVEIIKAALAKQNIALTD |
| | | QLDVLSAINSVEASPKSASFFGDICYAFEKTLQMRNSIPNTDEDYLAS |
| | | PVMNKRGEFYDSRSCDDALPQNADANGAYHIALKGLYLIKNVFDA |
| | | GGKELKISHEDWFKFAQSRNC |
| 85 | Cas12 | MYKDKTDKTKIIDSDLIKFINIAESTQLDSMSQDEAKELVKEFWGFTT |
| | | YFVGFYDNRKNMYTAEEKSTGIAYRLVNENLPKFIDNMEAFKKAIAR |
| | | PEIQANMEELYSDFSEYLNVESVQEMFQLDYYNMLLTQKQIDVYNA |
| | | IIGGKTDDEHDVKIKGINEYINLYNQQHKDDKLPKLKALFKQILSDRN |
| | | AISWLPEEFNGDQEVLNAIKDCYERLSENVLGDKVLKSLLGSLSDYSL |
| | | DGIFIRNDLQLTDISQKMFGNWCVIQNAIMQNIKHVAPARKHKESE |
| | | EDYEKRIAGIFKKVDSFSISFINDCLNEADPNNAYFVENYFATFGAVN |
| | | TPTMQRENLFALVQNAYTEVAALLHSDYPTAKHLAQDKVNVAKIKA |
| | | LLDAIKSLQHFVKPLLGKGDESDKDERFYGELASLWAELDTVTPLYN |
| | | MIRNYMTRKPYSQKKIKLNFENPQLLGGWDANKEKDYATIILRRDG |
| | | LYYLAIMNKESKKLLGKAMPSDGECYEKMVYKLLPGANKMLPKVFF |
| | | AKSRMEDFKPSKELVEKYNNGTHKKGKNFNIQDCHNLIDYFKQSISK |
| | | HEDWGKFGFNFSDTSTYEDLSGFYREVEQQGYKLSFARVSVSYISQL |
| | | VEEGKMYLFQIYNKDFSEYSKGTPNMHTLYWKALFDERNLADVVYK |
| | | LNGQAEMFYRKKSIENTHPTHPANHPILNKNKDNKKKESLFDYDLIK |
| | | DRRYTVDKFMFHVPITMNFKSSGSENINQDVKAYLRHADDMHIIGI |
| | | DRGERHLLYLVVIDLQGNIKEQYSLNEIVNEYNGNTYHTNYHDLLDV |
| | | CEEERLKARQSWQTIENIKELKEGYLSQVIHKITQLMVKYHAIVVLED |
| | | LNMGFMRGRQKVEKQVYQKFEKMLIDKLNYLVDKKADASVSGGLL |
| | | NAYQLTSKFDSFQKLGKQSGFLFYIPAWNTSKIDPVTGFVNLLDTRY |
| | | QNVEKAKVFFSKFDAIRYNKDKDWFEFNLDYDKFGKKAEGTRTKW |
| | | ALCTRGMRIDTFRNKEKNSQWDNQEVDLTAEMKSLLEHYYIDIHG |
| | | NLKDAISAQTDKAFFTGLLHILKLTLQMRNSITGTETDYLVSPVADEN |
| | | GIFYDSRSCGDELPENADANGAYNIARKGLMMIEQIKDAKDLDNLK |
| | | FDISNKSWLNFAQQKPYKNE |
| 86 | Cas12 | MNEADPNNAYFVENYFATFGAVNTPTMQRENLFALVLNAYTEVAS |
| | | LLHSYYPAEKNLAQDKANVAKIKALLDAIKSLQHFVKPLLGKGDESDK |
| | | DERFYGELASLWAELETVTPLYNMIRNYMTRKPYSQKKIKLNFENPQ |
| | | LLGGWDANKEKDYATIILRRNGLYYLAIMDKDSRKLLGKAMPSDGE |
| | | CYEKMVYKLLPGANKMLPKVFFAKSRMEDFKPSKELVEKYNNGTHK |
| | | KGKNFNIQDCHNLIDYFKQSISKHEDWGKFGFNFSDTSTYEDLSGFY |
| | | REVEQQGYKLSFARVSVSYISQLVEEGKMYLFQIYNKDFSEYSKGTP |
| | | NMHTLYWKALFDERNLADVVYKLNGQAEMFYRKKSIENTHPTHPA |
| | | THPILNKNKDNKKKESLFEYDLIKDRRYTVDKFMFHVPITMNFKSVG |
| | | SENINQGVKEYLHHADDMHIIGIDRGERHLLYLVVIDLQGNIKEQYSL |
| | | NEIVNEYNGNTYHTNYHDLLDAREDERLKARQSWQTIENIKELKEGY |
| | | LSQVIHKITQLMVKYHAIVVLEDLNMGFMRGRQKVEKQVYQKFEK |
| | | MLIDKLNYLVDKKADASVSGGLLNAYQLTSKFDSFQKLGKQSGFLFYI |
| | | PAWNTSKIDPVTGFVNLLDARYQNVEKAKAFFSKFDAIRYNKDKD |
| | | WFEFNLDYDKFGKKAEGTRTKWTLCTRGMRIDTFRNKEKNSQWD |
| | | NQEVDLTAELKSLLEHYYIDIHGNLKEAISTQTDKAFFTGLLHILKLTLQ |
| | | MRNSITGTETDYLVSPVADENGIFYDSRTCGDELPENADANGAYNI |
| | | ARKGLMMIEQIKNAEDLGNLKFDISNKAWLNFAQQKPYKNG |
| 87 | Cas12 | MDNNNLHAVDGYFATLGAVNTPTMQRENLFALIQNAYTDISDLLD |
| | | TPYPENKNLAQDKTNVAKVKALLDAIKSLQHFVKPLLGMGDESDKD |
| | | ERFYGELASLWTELDTVTPLYNMIRNYMTRKPYSEKKIKLNFENPQL |
| | | LGGWDANKEKDYATIILRRNGMYYLAIMDKDSKKLLGKTMPSDGE |
| | | CYEKMVYKLLPGANKMLPKVFFAKSRINDFKPSKKIVENYNNGTHKK |
| | | GKNFNINDCHDLIDYFKQSIDKHEDWSKFGFNFSDTSTYEDLSGFYR |
| | | EVEQQGYKLSFTNISVSFIDKLVDEGKMYLFQIYNKDFSDYSKGTPN |
| | | MHTLYWKALFDERNLADVVYKLNGEAEMFYRKKSINNTHPTHHAN |
| | | HPIQNKNKDNKKKESVFEYDLVKDRRYTEDKFLFHVPITMNFNSVG |
| | | AENINQQVRKYLQQADDTHIIGIDRGERHLLYLVVIDMQGNIKEQFS |
| | | LNEIVNEYNGNTYRTNYHDLLDVRADKRLKASQSWQTIENIKELKEG |
| | | YLSQAIHKITQLMVKYHAVVVLEDLNKGFMRGRQKVEKQVYQKFEK |
| | | MLIDKLNYLVDKHKDANETGGLLHALQLTSEFKNFKKSEYQNGFLFY |
| | | IPAWNTSKIDPVTGFVNRFDTRYTNAVEAQKFFRKFDEIRYNEEKD |
| | | WFEFEFDYDKFTQKAHGTRTRWTLCTHGKRLRSFRNPAKQYNWDS |
| | | EVVALTDEFKRILGEAGIDIHENLKDAISNLEGKRRKYLEPLMQFMKL |
| | | LLQLRNSRKNPEEDYILSPVADENGVFYDSRSCGDKLPENADANGA |
| | | YNIARKGLMLIRQIKEAKELGKVKYDISNKAWLNFAQQKPYKNE |
| 88 | Cas12 | MQKKAFEENQQNLRSIIAKKLTEDKAYANLFGKNLLESYKDKTDKTKI |
| | | IDSDLIKFINTAESTQLDSMSQDEAKEIVKEFWGFTTYFVGFFDNRK |
| | | NMYTAEEKSTGIAYRLINENLPKFIDNMEAFKKAIARPEIQADMEELY |
| | | SNFSEYLNVESIQEMFLLDYYNMLLTQKQIDVYNAIIGGKTDDEHDV |
| | | KIKGINEYINLYNQQHKDDKLPKLKALFKQILSDRNAISWLPEEFNSD |
| | | QEVLNAIKDCYERLSENVLGDKVLKSMLGSLADYSLDGIFIRNDLQLT |
| | | DISQKMFGNWSVIQNAIMQNIKHVAPARKHKESEEEYENRIAGIFK |
| | | KADSFSISYIDACLNETDPNNAYFVENYFATLGAVDTPTMQRENLFA |
| | | LVQNAYTEITALLHSDYPTEKNLAQDKANVAKIKALLDAIKSLQHFVK |
| | | PLLGKGDESDKDERFYGELASLWAELDTMTPLYNMIRNYMTRKPYS |
| | | QKKIKLNFENPQLLGGWDANKEKDYATIILRRNGLYYLAIMNKDSKK |
| | | LLGKAMPSDGECYEKMVYKLLPGANKMLPKVFFAKSRMEDFKPSK |
| | | ELVEKYNNGTHKKGKNFNIQDCHNLIDYFKQSIDKHEDWSKFGFKF |
| | | SDTSTYEDLSGFYREVEQQGYKLSFARVSVSYINQLVEEGKMYLFQIY |
| | | NKDFSEYSKGTPNMHTLYWKALFDERNLADVVYKLNGQAEMFYRK |
| | | KSIENTHPTHPANHPILNKNKDNKKKESLFEYDLIKDRRYTVDKFMF |
| | | HVPITMNFKSVGSENINQDVKAYLRHADDMHIIGIDRGERHLLYLVV |
| | | IDLQGNIKEQFSLNEIVNDYNGNTYHTNYHDLLDVREDERLKARQS |
| | | WQTIENIKELKEGYLSQVIHKITQLMVKYHAIVVLEDLNMGFMRGR |
| | | QKVEKQVYQKFEKMLIEKLNYLVDKKADASVSGGLLNAYQLTSKFDS |
| | | FQKLRKQSGFLFYIPAWNTSKIDPITGFVNLLDTRYQNVEKAKAFFSK |
| | | FDAIRYNKDKEWFEFDLDYDKFGRKAEGTRTKWTLCTRGMRIDTFR |
| | | NKEKNSQWDNQEIDLTAEMKSLLEHYYIDIQGNLKEAICTQTDKAFF |
| | | TGLLHILKLTLQMRNSITGTQTDYLVSPVADENGIFYDSRSCGDSLPE |
| | | NADANGAYNIARKGLMLIEKIKNAEDLDTIKFDISNKAWLNFAQQK |
| | | PYKNG |
| 89 | Cas12 | MLENEQEAEKIKNKLDDIMNIYHWIKIFLVDEEIEKDMDFYSEIEDIY |
| | | EELSPLVSLYNRVRNYVTQKPYSQEKMKLNFGSPTLADGWSKSKEFS |
| | | NNAIIMLKDGKYYIGIFNIRNKPNKEVIEGRNNRINDSDYKKMVYRLL |
| | | PGANKMLPKVMFSKKGIEYYNPSQYILSGYNSKKHIKSNENFDINFC |
| | | HDLIDFFKESINKNEEWKNFDFKFSDTESYNDISEFYREVEQQGYKIE |
| | | WVYISEQDIEQLEKNGQLYIFQIYNKDFAKKSIGNKNLHTMYLENLFS |
| | | EENLKDVVLKLNGEAEIFFRKSSIKKPIVHKAGSILVNKCIEDETGNKV |
| | | SFPDDIYNEIYQYMNGMTDVLSERAQNYYEKVKHSVSKQDIVKDYR |
| | | YTVDKYFIHLPITINFKASSFMPINDIALKYIAKRDDIHIIGIDRGERNLI |
| | | YVSVIDLQGNIVYQKNYNVVNGYDYKAKLRETEIQRDNARKNWKEI |
| | | GKIKQLKEGYLSLVVHEIAQLIVKYNAIVVMEDLNMGFKRGRFKVER |
| | | QVYQKFENMLINKLNYLVDKNKKVDEDGGLLRGYQLTYVPGQKEH |
| | | VGKQCGFIFYVPAAYTSKIDPTTGFVSIFNNKVNAKEFVTKFDSIKYN |
| | | KNMKMFELKFDYNNFETYNITLAKSKWTIYTNGIRLKREYNNGRWN |
| | | KITEVDLTKEMANTLKKYDIEFENNEEILKSISQLDEKNQRNICNEIKEI |
| | | IKLIVQLRNSMPDNGSKDNEYDKIISPVLNENNYFYDSSEVCDNSAP |
| | | ENADANGAYCIAMKGLYQVIQIKENWSQDSNPKNILGIKHYEWFD |
| | | FMQNKRYL |
| 90 | Cas12 | MAKNTIFTQFTGLYPVSKTLRFELKPMGKTLEKIKETGVIENDKKRHN |
| | | DYFDAKKIIDKYHKYFIDAALSKFSRIDWNPLKEAIEGSLDRSDASKKK |
| | | LEKTQTEFRKKIAKALTTHDHYKELTASTPKDLFLKVFPDHFGKQPAI |
| | | DTFDGFSSYFTGFQENRQNIYSDEAISTAIPYRLVHDNFPKFLSNIEVY |
| | | KTLKDNAPSVLSDAENELKDFLNGKPLANIFELNAYNDVLTQSGIDFF |
| | | NQVIGGISGEGGEKKTRGINEFSNLYRQQHPEFAQKRLATKMIPLYK |
| | | QILSDRETKSFILESYSTDSQVQESVKEFFESQILNCDIAGRKVNVLNE |
| | | LTSLIKRIAEFDLGSIYINQEELSNISLELFKSWNTINAVLFKNAENRIGS |
| | | AEKAANKKKIDAWMKSNEFSIATLNLAIAESDSEEISRVKIESYWNDF |
| | | EAKVQSILCGDNRRNLDEFLSATFNENNALREDSEIIGKLKAFLDALIE |
| | | IMHSIKPLISDAENRDLSFYNELMPLYDQLSLVVPLYNKIRNYATQKL |
| | | TESEKFKLNFDNPTLADGWDQNKEDANTAILLLKNGLYYLGIMNAK |
| | | NKPKIKDFKTSESEDCYDKMVYKLLPGPNKMLPKVFFSEKGLATFKP |
| | | PKDILDGYNAGKHKKGDLFDIGFCHQLIDFFKESIAKHPDWKKFDFK |
| | | FSDTSSYEDISGFYKEVTDQGYKITFSKIPTSQIDEWVNEGKLFLFQIY |
| | | NKDFAPGAKGSPNLHTLYWKSVFSPENLKDVVVKLNGEAELFYRPS |
| | | SVKKPYSHKVGEKLVNRIGKDGLPLPESVFGELFRYFNGKLDGELSDE |
| | | AKRYLDVAVVKDVKHEIVKDRRYTQDKFEFHVPLTLNFKADSKNEY |
| | | MNERVRHFLKDNPDVNIIGIDRGERHLLYMTLINQKGEILKQKSFNI |
| | | VESVNYQAKLVQREKERDAARKSWSSVGKIKDLKEGFLSQVIHEITT |
| | | TMIENNAIVVLEDLNFGFKRGRFCVERQVYQKFEKMLIDKLNYLVFK |
| | | NKPEGDVGGVLKGYQLAEKFDSFQKLGKQSGFLFYIPAAYTSKIDPT |
| | | TGFANLFNMTELTSAEKKKDFLSHFDDITYDGKNDRFLFGFDYKNFK |
| | | CFQTDFIKKWTVYTQGKRIVYDKESKSAKEIFPVEIIKAALAKQNIALT |
| | | DQLDVLSAINSVEASPKSASFFGNICYAFEKTLQMRNSIPNTDEDYLV |
| | | SPVMNKRGEFYDSRSCDDALPQNADANGAYHIALKGLYLIKNVFDA |
| | | GGKDLKISHEDWFKFAQSRNC |
| 91 | Cas12 | MRPVLQLTDTEDKLSQNKPAVGKIKALLDAFKDLQHFIKPLLGSGEE |
| | | NEKDELFYGAFQLIWDELDTVTPLYNKVRNWLTRKPYSTEKIKLNFD |
| | | NAQLLGGWDVNKEPDCTGVLLRKDGFYYLGIMNKKSNRIFDADVT |
| | | PADGICYEKIDYKLLPGANKMLPKVFFSKSRIDEFAPSEAILSSYKRGT |
| | | HKKGADFSLSDCHRLIDFFKASINKHEDWSKFGFQFSDTKTYEDISGF |
| | | YREVEQQGYMLSSHQVSEAYINQMVEEGKLFLFRIWNKDFSEYSKG |
| | | TPNMHTLYWRMLFDERNLADVVYKLNGQAEVFYRKASIKAENQIM |
| | | HPAHHPIENKNTLNEKRSSTFDYDLVKDRRYTVDKFQFHVPITINFKA |
| | | IGQ.TNVNPIVHETIRRGGFTHVIGIDRGERHLLYLSLIDLKGHIVKQM |
| | | TLNEIINEYNGLAHKTNYYDLLVKREGERTTARRSWDTIENIKELKEG |
| | | YLSQVIHIISKMMVEYNAIVVLEDLNMGFMRGRQKIERQVYEKFEK |
| | | MLIDKLNCYIDKQADSQSEGGLLHPIQLANKFESFRKLGKQSGCLFYI |
| | | PAWNTSKIDPVTGFVNLFDTRYETREKAKLFFSHFQRICFNAEKDWF |
| | | EFSFDYNDFTTKAEGTRTQWTLCSYGTRIRNFRNPLQNHQWDDEEI |
| | | VLTEAFKALFDKYDIDIHANLKEAINAQTDAQFFKDLMGLMKLLLQ |
| | | MRNSKTNSEVDYLLSPVADEHGRFFDSRAGAGSLPDNADANGAYN |
| | | IARKGLWVIRKIQETPEGEKLSLAITNKEWLEFAQTKPYLND |
| 92 | Cas12 | MNLNTYFSQFTGLYPVSKTLRFELKPMGKTLEKIKETGIIENDKKRHN |
| | | DYFDAKKIIDKYHKYFIDAALSKFPCIDWNPLKEAIERSLDRSDASKKK |
| | | LEKTQTEFRKKIAKALTTHGHYKELTASTPKDLFLKVFPDHFGKQPAI |
| | | DTFDGFSSYFTGFQENRQNIYSDEAISTAIPYRLVHDNFPKFLSNIEVY |
| | | NILKDNAPSVLSDAENELKDFLNGKPLANIFELNAYNDVLTQSGIDFF |
| | | NQVIGGFSGEGGEKKTRGINEFSNLYRQQHPEFAQKRLATKMIPLYK |
| | | QILSDRETKSFILESYSTDSQVQESVKEFFESQILNCDIAGRKVNVLKEL |
| | | SSLIKRITEFDLGSIYVNQEELSSISLELFKSWNTINAILFKNAENRIGSA |
| | | EKAANKKKIDAWMKSNEFSIATLNLAIAESDSEEISRVKIESYWNNFE |
| | | AKVQSILCGDNRRNLDEFISATFNENNALREDSKVIEKLKAFLDALIEI |
| | | MHSIKPLISDAENRDLSFYNELMPLYDQLSLVVPLYNKIRNYATQKLT |
| | | ESEKFKLNFDNPTLADGWDQNKEEANTAILLLKNGLYYLGIMNAKN |
| | | KPKIKDFKTSESEDCYDKMVYKLLPGPNKMLPKVFFSEKGLATFKPPK |
| | | DILDGYNAGKHKKGDLFDIGFCHQLIDFFKESIAKHPDWKKFDFKFS |
| | | DTSSYEDISGFYKEVTDQGYKITFSKIPTPQIDEWVNEGKLFLFQIYNK |
| | | DFAPGAKGSPNLHTLYWKSVFSPENLKDVVVKLNGEAELFYRPSSVK |
| | | KPYSHKVGEKLVNRIGKDGLPLPESVFGELFRYFNGKLDGELSDEAKR |
| | | YLDVAVVKDVKHEIVKDRRYTQDKFEFHVPLTLNFKADSKNEYMNE |
| | | RVRHFLKDNPDVNIIGIDRGERHLLYMTLINQKGEILKQKSFNIVESV |
| | | NYQAKLVQREKERDTARRSWSSVGKIKDLKEGFLSQVIHEITTTMIE |
| | | NNAIVVLEDLNFGFKRGRFCVERQVYQKFEKMLIDKLNYLVFKNKPE |
| | | GDVGGVLKGYQLAEKFDSFQKLGKQSGFLFYIPAAYTSKIDPTTGFA |
| | | NLFNMTELTSAEKKKEFLSHFEDITYDGKNDRFLFSFDYKKFKCFQTD |
| | | YIKKWTVYSQGKRIVYDKESKSAKAISPVEIIKAALAKQNIALTDQLDV |
| | | LSAINSVEASRETASFFGDICYAFEKTLQMRNSIPNTDEDYLVSPVM |
| | | NKKGEFYDSRSCGDSLPKNADANGAYHIALKGLYLIKNVFDAGGKDL |
| | | KISHEDWFKFAQSRNR |
| 93 | Cas12 | MQTLFENFTNQYPVSKTLRFELIPQGKTKDFIEQKGLLKKDEDRAEK |
| | | YKKVKNIIDEYHKDFIEKSLNGLKLDGLEEYKTLYLKQEKDDKDKKAFD |
| | | KEKENLRKQIANAFRNNEKFKTLFAKELIKNDLMSFACEEDKKNVKE |
| | | FEAFTTYFTGFHQNRANMYVADEKRTAIASRLIHENLPKFIDNIKIFEK |
| | | MKKEAPELLSPFNQTLKDMKDVIKGTTLEEIFSLDYFNKTLTQSGIDIY |
| | | NSVIGGRTPEEGKTKIKGLNEYINTDFNQKQTDKKKRQPKFKQLYKQ |
| | | ILSDRQSLSFIAEAFKNDTEILEAIEKFYVNELLHFSNEGKSTNVLDAIK |
| | | NAVSNLESFNLTKIYFRSGTSLTDVSRKVFGEWSIINRALDNYYATTY |
| | | PIKPREKSEKYEERKEKWLKQDFNVSLIQTAIDEYDNETVKGKNSGK |
| | | VIVDYFAKFCDDKETDLIQKVNEGYIAVKDLLNTPYPENEKLGSNKD |
| | | QVKQIKAFMDSIMDIMHFVRPLSLKDTDKEKDETFYSLFTPLYDHLT |
| | | QTIALYNKVRNYLTQKPYSTEKIKLNFENSTLLGGWDLNKETDNTAII |
| | | LRKENLYYLGIMDKRHNRIFRNVPKADKKDSCYEKMVYKLLPGANK |
| | | MLPKVFFSQSRIQEFTPSAKLLENYENETHKKGDNFNLNHCHQLIDF |
| | | FKDSINKHEDWKNFDFRFSATSTYADLSGFYHEVEHQGYKISFQSIA |
| | | DSFIDDLVNEGKLYLFQIYNKDFSPFSKGKPNLHTLYWKMLFDENNL |
| | | KDVVYKLNGEAEVFYRKKSIAEKNTTIHKANESIINKNPDNPKATSTF |
| | | NYDIVKDKRYTIDKFQFHVPITMNFKAEGIFNMNQRVNQFLKANPD |
| | | INIIGIDRGERHLLYYTLINQKGKILKQDTLNVIANEKQKVDYHNLLDK |
| | | KEGDRATARQEWGVIETIKELKEGYLSQVIHKLTDLMIENNAIIVME |
| | | DLNFGFKRGRQKVEKQVYQKFEKMLIDKLNYLVDKNKKANELGGLL |
| | | NAFQLANKFESFQKMGKQNGFIFYVPAWNTSKTDPATGFIDFLKPR |
| | | YENLKQAKDFFEKFDSIRLNSKADYFEFAFDFKNFTGKADGGRTKWT |
| | | VCTTNEDRYAWNRALNNNRGSQEKYDITAELKSLFDGKVDYKSGK |
| | | DLKQQIASQELADFFRTLMKYLSVTLSLRHNNGEKGETEQDYILSPV |
| | | ADSMGKFFDSRKAGDDMPKNADANGAYHIALKGLWCLEQISKTD |
| | | DLKKVKLAISNKEWLEFMQTLKG |
| 94 | Cas12 | MIDNTKEKKEGSVFDGFTRKYQLSKTLRFELRPILNTPKMLDDEQVIK |
| | | NDETRRKKYEAVKPWFDQLHREFIEDALKSFKFKNLAIYQDTFQTW |
| | | QKDRKSKQKKDTLVKIEVGLREEIVRRFEEVANIWVRSEQYKLLGIKK |
| | | EGLGMLFEAGVFRLLKERFKNEKDTTVDGNNIFDEWTRWTGYFKKF |
| | | FETRKNFYKSDDTSTAIAYRVINQNLRRFCENIQIFEKISEKIEFSEVEK |
| | | SFDISCAGIFSLAYYNACLLQGGIDTYNKIIGGEVDEKDKKIPGINELIN |
| | | KYRQDNSGEKIPFLKQLDKQIHSAKEAFIESIETNKELVGKLKTFYENA |
| | | EVKIQSFRNLIADIVTDYSGYDIDKIYLTKEAVSHNASRWFASFESFER |
| | | DLFAVVAEKQNKLVYELLRTHKNDSKISDKDGKFSFPDFIKCSHIKRA |
| | | LEKQEGRIWKGEYYEDIVDFEKIKDVFTQFLCVFKFELEQQFFRKTTS |
| | | AQTGEQTKIGYEIFVTKINELITRENPVIDLEEKIAIKNFADATLLIYQIA |
| | | KYFAVEKRRGWLDNYDLDDRFYKSSDIGYLNFYRDAFEQIVRPYNLF |
| | | RNYLTKKPYNTNKWVLSFENPTLADGWDKNKEKTNAAVILRKDGR |
| | | YYLGIIKEDCKSLFADRYSKEMSEGIESGSFQKMAYKFFPEASKMIPK |
| | | CSTQTKNVKEHFRKSSSDYNLFHEKDYKISVAITKNIYELNNVFYRKD |
| | | NIEESFVPKNDFEKKLGVKKFQRQYLEISRDNNGYKQALAQWIEFCI |
| | | RFLKAYKSTTIFDYSRLREAKEYESLDAFYQDINALTYNISFVPISEQYIK |
| | | EKNDNGELFLFEIYNKDWSLGPMDKNRKRTKNLHTLYFEQLFSKEN |
| | | EQENFLFQLNGEAELFFRPKTEEKRLGYKVWDAGEKKWVKAKEKED |
| | | GAVIDRKRYAKDIILFHCPITLNRVSESKTKREMDVEIREVLSSTPGVH |
| | | IIGVDRGEKHLAYYSVIDQNGKIIETDTLNSIGKDGRGKPVEYASKLEK |
| | | RAQEREASRRDWEEVEAIKDLKKGYISQVIRNLADLIIKHNAIIVFEDL |
| | | NMRFKQIRGGIEKSAYQQLERALIDKLSFLVKKGEEDPKQTGHILRAY |
| | | QLAAPVIAFKDMGKQTGLIFYTQAGYTSKTCPECGYRRNIKCLFENIE |
| | | QAKTLIENLESINYNKKEDVFQISYSLEKLSSKDQKKEKKVSNELYAKT |
| | | LKKDIFILTTKNALRYKWYDRYSEKAKVAKRGIDEYKGEVNESETKKG |
| | | VVKEFNLTEYLKGLLKTYEIDYEHGGIREQILSVARGREFYKDFLYALFL |
| | | LTETRHSISGRNTDYIQCPECEFDSRKGFKDIKEFNGDANGAYNIARK |
| | | GIMILEKIKQFKKDNDGNLEKMGWGDLSISIEEWDKFTQKE |
| 95 | Cas12 | MKNFQDFTNLYELSKTLRFELKPIGGTKKLIEEKNILKLDKKKRENYEK |
| | | VKPYFNKIHQEFINFALRNPNFDFSQFEEKYLNWLKDKKNKDLLKEK |
| | | ESIDKIFLEKIGKLFENSVKDFLKENGFESIVKEEDQNLKFFRRKEIFEVL |
| | | QEKYGSELETQMVNKDGEIKSIFNGWEKWLGYFDKFFNTRDNFYKT |
| | | DGTSTAIATRIIKDNLKIFLENIVAFGKIKNKKIDFSEVEKNFSVSIDTFF |
| | | EINNFNNCFLQDGIDFYNKVIGGETLENGEKLKGLNEIINKYRQDTGE |
| | | KIPYFKKLQKQILSEKDGVFIDKIEDDGGFYEVLKNFYKNAAEKEGFLK |
| | | NIFENFYTISDKNLEKIYFNKIAFNTISHKFGSALEFERILYEEMKKEKA |
| | | DGIKFEKKENKYKFPDFIQIIFIKRSLENYDSENLFWKERYYKSEENVD |
| | | GFLEKNNNNLWGQFCKILNFEFLNILKRRIIDEAGEEYEVGFEISKNIL |
| | | GEKLENFELNQENKGIIKDFADYSLALYSFGKYFAVEKGRNWDLNIDI |
| | | SDDFYGGEDGYIEKFYNTGYDEIVKPYNLMRNYISKKPWEDNKKWK |
| | | INFETSSLLSGWDKNLESNGSYIFQKGNKYYLGIINGSKPAKEILEKLYS |
| | | GDGEKIKRFIYDFQKPDNKNTPRMFIRSKKDSFSPAVEKYNLPINDIL |
| | | EIYDNGLFKTENKGNPNYKESLRKLIDYFKLGFSRHESFKHFNFVWK |
| | | DSKSYENIADFYRDVEKSCYKIDFEFLNFEELKKLTFEKHLYLFQIYNKD |
| | | FELDESLQEKGYNFKGEGQKNIHTKYFEALFLEENISRKSGAVFKLSG |
| | | GGEVFFRKKSIKAKKEKRNSVEVIKNKRYTECKYFLHFPIQVNFKEEIS |
| | | GNFNQEINKFLANNPDINVIGIDRGEKHLAYFSVINQKGEILESGSFN |
| | | KIENYNKNGEKLLFPEREIKEIHKDGSLIDLELVETGRKVDYVDYKLLLE |
| | | YKERKRLLQRQSWKEVEQIKDLKKGYISALVRKIADLIIKHNAIVIFEDL |
| | | NFRFKQIRGGIEKSIYQQLEKALIDKLNFLVNKNEINLEKAGSILKAYQ |
| | | LTVPVDSLKEIGKQTGVIFYTEAAYTSKIDPITGWRPNLYLKKNNSKIN |
| | | KENILKFDNIVFNSKENRFEFTYDLKKFFGKDSKFPAKTVNTVCSCVE |
| | | RFKWNRNLNNNKGGYIHYENLTDGKLANKEQKEDEFSNFKELFEKY |
| | | FIDINGNILEQIKNLDTKNNEKFFSSFIDLFTLVCQIRNTNQNAKGDE |
| | | NDFILSPVEPFFDSRKSQNFGKSLPKNGDENGAFNIARKGLIILNRISE |
| | | NPEKPDLLIFNADWDNFARNI |
| 96 | Cas12 | MSNNFQEFTQKYALSKTLRFELKPVGKTKEILEKEMPMYQIINADKN |
| | | IKAKYIQTKPFFDQLHRDFIKEAFENVELSGLSDFFENWKIYKQDKKA |
| | | NEKIYKKSAENLRKEVVSFLNAKGKDWAEKYHSSGLKKADIEILFEEGI |
| | | FKVLEIRYGTDTNSFITNATTGEITSIFQGWKGFTGYFLKFWNTRENY |
| | | YKTDGTSTAIATRIVDQNLPGYLENLEIFEKMKGKIDFESVRGDFSDF |
| | | EKIGTVEYYSTCLLQEGIDGYNRIIGGYTYENGEKIKGINEIINLYRQTH |
| | | KDEKVPFLKTLDKQIGSEKIAFMETIDTPEEFRKIFEEFVLKSSEKVVLL |
| | | KQCLNHLFENELTDGVFLSKESLNTISHKWIDIGNMKLFHESLFTILKK |
| | | EGAKYDSKEDEYKFPDFIRISDIKTALVKITTESFFWKNRYLYEKDENP |
| | | TGFLTSDNSLWEGFIQIFSHEFSSLFERTEKDEEGKDIQWGYDISLLNI |
| | | QKLLENNEYNPNDEKNKIIIKSFADDILRIYQMGKYFALEKKRQWNP |
| | | DNLEIGEFYSHPEIGYDKFYFDSYKIIVQGYNDIRNYLTKNPWSEEKW |
| | | KLNFENPTLANGWDKNKETDNSCIFLKRDNKFFLALMSRGNNQVF |
| | | DERNIQKFAQNIEQGKYEKMVYKYMKDVALGIPKATTQLNAVQEH |
| | | FFQSDKDYIITKGGSSIGEFIKPLRVTKRIFELNNRIYPKDNLGISFLRN |
| | | QVNKKEQKNYIKIFQKEFITLGGDEVVYKKAVHDWIDFCKEYTKSYPS |
| | | CAYFDYSGLKDTKEYSSIDEFYNDLDSFGYQISWQDISSSYIDELVESG |
| | | KLYLFEIYNQDFSNGKTGAKNLHTLYFEHIFSKENQEVNFPLKLNGQ |
| | | AELFFRPKSIEAKGENRKFNREIIAKKRYTEDKIFFHVPLTLNRTEGDIY |
| | | GFNTEINNFLAHNPDINIIGIDRGEKHLAYYSVIDQKGNIIESDSLNTV |
| | | NEINYGEKLTDTAEKRKQARQDWQAVEGIKNLKKGYISAVVHKLTD |
| | | LIIKYNAIVIFEDLNMRFKQIRGGIEKSVYQQLEKALIEKLNYLVEKGEI |
| | | NPEKAGHLLNAYQLTAPFETFKDMGKQTGIVFYTQAAYTSKIDPVT |
| | | GWRPHLYLKYSSAEQVKKEIAKFSNIIWNNTEKRFDFMYDIRNFSTQ |
| | | KEYPKNNIWTVCSSVERYRWDKTLNQNKGDYVHYKSITPEFEKLFSD |
| | | FQIDGTKNILEQINRMETKGNEKFFKSFIFFFGLICQIRNTNKADSDE |
| | | NKQDFILSPVVPFFDSRDSENTKNGLPRNGDENGAYNIARKGLIILQ |
| | | KINEFSDENGNCDKLGWKELSISQVDWDNYIKT |
| 97 | Cas12 | MEYDEHISLIESEEKADEMKKRLDMYMNMYHWAKAFIVDEVLDRD |
| | | EMFYSDIDDIYNILENIVPLYNRVRNYVTQKPYTSKKIKLNFQSPTLAN |
| | | GWSQSKEFDNNAIILIRDNKYYLAIFNAKNKPDKKIIQGNSDKKNDN |
| | | DYKKMVYNLLPGANKMLPKVFLSKKGIETFKPSDYIISGYNAHKHIKT |
| | | GENFDISFCRDLIDYFKNSIEKHAEWRKYEFKFSATDSYNDISEFYREV |
| | | EMQGYRIDWTYISEADINKLDEEGKIYLFQIYNKDFAENSTGKENLH |
| | | TMYFKNIFSEENLKNIVIKLNGQAELFYRKASVKNPVKHKKDSVLVN |
| | | KTYKNQLDNGDVVRIPIPDDIYNEIYKMYNGYIKESDLSGAAKEYLDK |
| | | VEVRTAQKEIVKDYRYTVDKYFIHTPITINYKVTARNNVNDMAVKYI |
| | | AQNDDIHVIGIDRGERNLIYISVIDSHGNIVKQKSYNILNNYDYKKKLV |
| | | EKEKTREYARKNWKSIGNIKELKEGYISGVVHEIAMLMVEYNAIIAM |
| | | EDLNYGFKRGRFKVERQVYQKFESMLINKLNYFASKGKSVDEPGGLL |
| | | KGYQLTYVPDNIKNLGKQCGVIFYVPAAFTSKIDPSTGFISAFNFKSIS |
| | | TNASRKQFFMQFDEIRYCAEKDMFSFGFDYNNFDTYNITMGKTQ |
| | | WTVYTNGERLQSEFNNARRTGKTKSINLTETIKLLLKDNEINYADGH |
| | | DVRIDMEKMDEDKNSEFFAQLLSLYKLTVQMRNSYTEAEEQEKGIS |
| | | YDKIISPVINDEGEFFDSDNYKESDDKECKMPKDADANGAYCIALKG |
| | | LYEVLKIKSEWTEDGFDRNCLKLPHAEWLDFIQNKRYE |
| 98 | Cas12 | MFGNWGVIQNAVMQNIKRVAPARKHKESEEDYEKRIAGIFKKADS |
| | | FSISYINDCLNEADPNNAYFVENYFATFGAVNTPTMQRENLFALVQ |
| | | NAYTEITALLHSDYPTEKNLAQDKANVAKIKALLDAIKSLQHFVKPLL |
| | | GKGDESDKDERFYGELASLWAELDTMTPLYNMIRNYMTRKPYSQK |
| | | KIKLNFENPQLLGGWDANKEKDYATIILRRNGLYYLAIMNKDSKKLL |
| | | GKAMPSDGECYEKMVYKLLPGANKMLPKVFFAKSRMEDFKPSKEL |
| | | VEKYNNGTHKKGKNFNIQDCHNLIDYFKQSIDKHEDWSKFGFKFSD |
| | | TSTYEDLSGFYREVEQQGYKLSFARVSVSYINQLVEEGKMYLFQIYNK |
| | | DFSEYSKGTPNMHTLYWKALFDERNLADVVYKLNGQAEMFYRKKSI |
| | | ENTHPTHPANHPILNKNKDNNKKESLFEYDLIKDRRYTVDKFMFHV |
| | | PITMNFKSSGSENINQDVKAYLCHADDMHIIGIDRGERHLLYLVVIDL |
| | | QGNIKEQFSLNEIVNDYNGNTYHTNYHDLLDVREDERLKARQSWQ |
| | | TIENIKELKEGYLSQVIHKITQLMVKYHAIVVLEDLNMGFMRGRQKV |
| | | EKQVYQKFEKMLIEKLNYLVDKKADASVSGGLLNAYQLTSKFDSFQK |
| | | LGKQSGFLFYIPAWNTSKIDPVTGFVNLLDTRYQNVEKAKSFFSKFD |
| | | AIRYNKDKEWFEFNLDYDKFGKKAEGTRTKWTLCTRGMRIDTFRNK |
| | | EKNSQWDNQEVDLTAEMKSLLEHYYIDIHSNLKDAISAQTDKAFFT |
| | | GLLHILKLTLQMRNSITGTETDYLVSPVVDENGIFYDSRSCGDELPEN |
| | | ADANGAYNIARKGLMMIEQIKDAKDLDNLKFDISNKAWLNFAQQK |
| | | PYKNG |
| 99 | Cas12 | MSNLYSNLHNLYQVQKTLRFELKPQGKTKENMEKVGILKADEHRAE |
| | | IYSKVKKYCDEYHKLFIDKSLSNIELNGIDRYYELYSINNRDDKQKEELD |
| | | QLEASLRKQISDAFKKSAEYKGLFQKDIITSYLVTMYKENQEKMQDI |
| | | GEFNRFTTYFTGYNKNRENMYSEEDKSTAISYRLINENLPTFIDNIKIY |
| | | KKIVSLMPEDIEKIYKDLEEYIQVDSIDEIFNISYYNDVLTQRGIECYNIL |
| | | ISGRTKNDGDKIKGLNEYINEFNQTHNEKIPKLQELYKQILSDAESASF |
| | | KIDVIKNDKELMNLIEVYYANILPILNKIEDLFTRISNYNLELILVNNDG |
| | | TLSTLSNMVFNEWSYIKGAISEKYDEEYSGKEKYGTEKYAQKKQEYLK |
| | | KQKIYSLKFLNDCIGNNAICEYLKNYIIQNKNIETIKEDYNEVQNIKVE |
| | | DDTKELIKDEKSIEKIKKFLDDVKSLQEFVKLVIPKDRTVEKDAKFYSEL |
| | | TPYYEKIKEIIPLYNKVRNYVTQKPYSTEKIKLNFECPTLLKGWDANKE |
| | | EANLGVILLKEGKYYLGIINPYCKKIFEVDEKDSNEQNNYKKMEYKLL |
| | | PGPNKMLPKVFFSNSRIEEFNPSKELQEKYNKGYHKKGKDFDINFCH |
| | | ELIDFYKQSVNKHEDWKKFNFKFKDTSEYNDISEFYREVEKQGYKIEY |
| | | TEYSEKYINELVDRGELYLFQIYNKDFSEYSKGKENLHTLYWKAVFDP |
| | | DNIMNPVYKLNGNAEVFYRKKSLEMKVTHPANQPIANKNISTIEAG |
| | | RSTSTFKYDLIKDKRYTMDKFQFHVPITMNFKSERLFNINQIVNKYLK |
| | | YNDDIHVIGIDRGERNLLYVCVIDKNEKIVYQKSLNEIVSEYNNNRYT |
| | | TDYHGLLDRKEKEREIAREDWKNIENIKELKEGYMSQVIHILVELMKK |
| | | YNAIIVIEDLNKGFKNSRIKVEKQVYQKFEKMFIDKLNYLVFKDEDKM |
| | | NEGGVLNAYQLTNKFESFTKLGKQSGILYYIPAWCTSKIDPTTGFINR |
| | | FYLKYENFDKSKEFVNRIDDIRYNEKENLFEFDIDYSKFTDRLNDTKNK |
| | | WTLCSYGERILTQKNANGEWLDRRIQLSIEFKKLFEKYGINLNNIKDSI |
| | | LKLDKDNLEFYKGNGESLGFIQLFKLMVQMRNSLTGKEEDNLISPVK |
| | | NQHGKFFNTSEKIEGLPIDADANGAYNIARKGFMLVEQMKNVEDE |
| | | KLNKIKYNITEKEWLNYVQNRGM |
| 100 | Cas12 | MARIFEDFKRLYPLSKTLRFEAKPIGATLDNIVKSGLLDEDEHRAESYV |
| | | KVKKLIDEYHKVFIDRVLDNGCLPLKNEGHNNSLTEYYDSYVSKSQN |
| | | EDAKKAFEENQQNLRSIIAKKLTEDKAYANLFGKNLIESYKDKTDKTKI |
| | | IDSDLIQFINTAESTQLNSMSQDEAKELVKEFWGFTTYFVGFFDNCK |
| | | NMYTAEEKSTGIAYRLINENLPKFIDNMEAFKKAIARPEIQANMEEL |
| | | YSNFSEYLNVESIQEMFLLDYYNMLLTQKQIDVYNAIIGGKTDDEHD |
| | | VKIKGINEYINLYNQQHKDDKLPKLKALFKQILSDRNAISWLPEEFNS |
| | | DQEVLNAIKDCYERLSENVLGDKVLKSLLGSLADYSLDGIFIRNDLQLT |
| | | DISQKMFGNWGVIQNAIMQNIKRVAPARKHKESEEDYEKRIAGIFK |
| | | KADSFSISYINDCLNEADPNNAYFVENYFATFGAVNTPTMQRENLF |
| | | ALVQNAYTEVASLLHSYYPAEKKLAQDKANVAKIKALLDAIKSLQHFV |
| | | KPLLGKGDESDKDERFYGELASLWAELDTVTPLYNMIRNYITRKPYS |
| | | QKKIKLNFENPQLLGGWDANKEKDYATIILRRNGLYYLAIMDKDSRK |
| | | LLGKAMPSDGECYEKMVYKLLPGANKMLPKVFFAKSRMDDFKPSK |
| | | ELIEKYNNGTHKKGKNFDIQDCHNLIDYFKQSIDKHEDWSKFGFNFS |
| | | DTSTYEDLSGFYREVEQQGYKLSFARVSVSYINQLVEEGKMYLFQIY |
| | | NKDFSEYSKGTPNMHTLYWKALFDERNLADVVYKLNGQAEMFYRK |
| | | KSIENTHPTHPANHPILNKNKDNKKKESLFEYDLIKDRRYTVDKFMF |
| | | HVPITMNFKSVGSENINQGVKEYLHHADDMHIIGIDRGERHLLYLVV |
| | | IDLQGNIKEQYSLNEIVNEYNGNTYHTNYHDLLDAREDERLKARQS |
| | | WQTIENIKELKEGYLSQVIHKITQLMVKYHAIVVLEDLNMGFMRGR |
| | | QKVEKQVYQKFEKMLIDKLNYLVDKKADASVSGGLLNAYQLTSKFD |
| | | SFQKLGKQSGFLFYIPAWNTSKIDPVTGFVNLLDARYQNVEKAKAFF |
| | | SKFDAIRYKRIRTGLSLISTMTSLVKKQKVQGLSGFYAPEECVLILSEIK |
| | | KKTHNGIIRKLT |
| 101 | Cas12 | MISLNYFQNQYAVAKTLCLELRPIEKTMEYIISSGILKEDEHRNESYKL |
| | | VKKIIDDYHKAYIELSLSRFELKITSCSKNDALEDFYCQYLANSQEEKDK |
| | | NIFKKTQDNLRKQIAKHLTQGEAYKRIDKKELIQEDLLEFVAADPDAA |
| | | NKKILINEFRDFTTYFTGFYENRKNMYSEEAQSTAIAYRIIHENLPKFID |
| | | NMGTFKQLMQSSITDILPQIFDNFKKDLEVSSIQEIFDLNYFNKVLTQ |
| | | KQIDIYNAIIGGKSLNENSRIQGLNEYINLYNQQHKENKLPLLKLLFKQ |
| | | ILSDRNSLSWLPEAFETDKQVLHAVRKCYANLKESVLHEAGLVQLLSS |
| | | LPSYDSTRIYIRNDQALTTISQKLFGDWGIIPHAIKERLKKDISAKRKET |
| | | EEAYLERIEKAFKQADSYTIAYINDSLKEIGVDKKNIEDYFIHLGAICTE |
| | | GQEQENILQRIASAYSQAQPLLEEKVPVHKNLMQDKDSVELIKSLLD |
| | | ELKNLQHFIKPLLGKGSESDKDERFYGEFVGLWNELDQITTLYNKVR |
| | | NYVTRKPYSIEKFKINFQNATLLKGWDRNKERDNTSIILRKNGLYYLAI |
| | | MRKEYNKVFEKYPAGTEENCYEKMEYKLLPGANKMLPKVFFSKSRI |
| | | NEFNPSPQLLQNYQMGTHKKGDQFKKEDCHALIDFFKTSIEKHED |
| | | WKNFNFQFSPTSVYEDMSGFYREVEQQGYKLVFRSIDAEYIDKLVEE |
| | | GKIFLFQIYNKDFSPFSKGTPNLHTLYWKMLFDERNLNNVVYKLNGE |
| | | AEIFFRKKSITYTHPTHPAEIPIKNKNVQNKKKESVFQYDLIKNHRFTI |
| | | DSFQFHVPITMNFKNAGLSNLNEQ.VYTYLRENKDAHIIGIDRGERHL |
| | | LYLVVIDRYGRIVKQFSLNEIVNEYHGNTYTTNYHDLLDKREEARQQ |
| | | ARQSWQSIENIKELKEGYLSQVVHKIANLMVEYHAIVVLEDLNIGFM |
| | | RGRQKVEKQVYQKFEKMLIDKLNYLVDKKKAPEADGGLLKAFQLTN |
| | | QFESFQKLGKQSGFLFYVPAWNTSKIDPCTGFTNLLDTRYENIAKAQ |
| | | KFFRTFDAIRYNAVKDYFELELDYDKFHKRAEGTQ.TKWTLCTYGTRIK |
| | | TFRNPENNNQWDNVEINLTEEFKKLFKQFGINLSGDLQQAICAQTE |
| | | KSFFESLLRLLKLTLQMRNSITGTDVDYLLSPVQNAEGYFYDSRKGDK |
| | | SLPANADANGAYNIARKGLWVIQQIKQTPQGQKAKLSISNKEWLKF |
| | | AQEKPYLKD |
| 102 | Cas12 | MKTFENFTNLYSLPRTLRFELKPLYKTKELIDSKQELFPKDKRIDEIYQN |
| | | IIKPCLNELHSDFIEKSMENKDFQNIPDNILKIYSNEKNIDDFKNIEKDL |
| | | IKQINWFLKSNKTFFAENYSDLLGKNSIDIIIKVFWEKIYKKDDSWKIF |
| | | LYNDLLWKSYEELINIYFKWFSTYLSNFNKNRENLYDKKNEAKVWSV |
| | | SGRTIWENFPRFLQNCINFRDKLEKLNLSIEQKDIFITNNFWKCISQK |
| | | QIDYYNKIIWQINSKTNEFNQKNGLKWNKKLPKLLLLHKQILWKSEN |
| | | ENILNFINNIIQ.TDFELEQ.EIKIINKDIFQ.RIDFIKKSIVSNIEDFELEKIFIK |
| | | KNRLKDISSLLMDNYSVLEKLLPEFNEEWKIIKENELVNLSKIKKSFENI |
| | | DLKDLKNIFKKEYFDESKDWFKLFLNWIYNHFSDLENNIKHTHKLVQ |
| | | DKLISWNFSENIQKSEKNINLRDEIFVSSKWLLKAYLDSILALDRFVH |
| | | MFDYWEQKDFDSNFYNNIEEYSINFSPFKTYNAVRNYLTKKNYSTD |
| | | KIKLNFDYPDFLGSNSLWKYAFIYKDSKWFYYLWVLDHSNSQSKYKP |
| | | QILKNNTEFYQLEYKQIKFNTLAWKWYIRDFWVKYSEDENCIINLKTL |
| | | IKKQYLERYPVLKEIVDFQTDDKKIFDAKVKTILEQAYSINFVNIDKNYI |
| | | LEENNNWNLHFFQIYNKDFSENKKINSMENLHTMYFKALFEKENFN |
| | | WWACFKLNSQWAEIFFREKSINEKKVKDLKTRNENAIEKKRYTENK |
| | | VFLHLPITLNFINKWYSKYSFWYINDSVKKYIKENKISIIWIDRWEKNLI |
| | | YFSMINENLEIIELKSLNSLILKVSDLEEKEVNYFEKLSKKEWNRNKER |
| | | KDWDEIETIKELKEWYISQIVDNLVKLIVKHNAIVVMEDLNSGFKRW |
| | | RQKIEKQIYQKFELALAKKLNFTVDKNKKHDELWWIYKAYQLTPQIE |
| | | NFQDIYSQTWIIFYTQAAYTSVTCPNCSFRKNIYQKYENESKFKEFFK |
| | | KYILEIKFEDNCFIIKYKIDEKIDKKKNKLKKLEFQVNTKNQIRLKFEKSL |
| | | KWKWWETKEFNITEKFKEIFEKHKLDLWNLKEELLAWNWEIQLYKD |
| | | FMFYFNLLLQLRNSKENDNWWYISCPSCWFHSGNWFQWFSYNW |
| | | DANWAYNIARKWRIILDKIKKDEKNLWITNVEWDNYYQK |
| 103 | Cas12 | MAVRSVKLKLLVPRDGSAESVRKRKALWATHQFVNDAAAAYAELLL |
| | | EMRQEDVCRGTDDHGKDVIEPAAHWQAKLRARLAAKQLPPVAVA |
| | | EALPLLKAFYGSRLIKSFVANDKGVAGTGNATDLNTWLSGLVDPASV |
| | | AGEKTELRKQLLAELPLCEAADADFEGAARKMLAKSDAREALLEGP |
| | | GTGVGWPAAYNANPTDSVWLDMLHKAAAKARLELADTTVSELKK |
| | | LGVFPLLQAASSNRVFGSGVLNPFERMAAAQAAAALLPWETKRHE |
| | | MRKRRDKFADQLNQWDTEFGASHATALAAIRAFEAEESERARRESL |
| | | GNEGTGYRIGGRELRDAWTLLRDWLKGHSTATAAAREDKVRELQA |
| | | KQGRSFGSHRLLSWLAKPAQQWLADHSAGDVVTRIAVRNARQRK |
| | | LDTARTLPIWTGADAVKHPRFANFDPPNNTNQPGFDLRAGTQKGR |
| | | LTLRLSLLTERADGLLLAQDHDFQLVPSRQMAEIVLHKDGKERALSW |
| | | QSQDGIGRQVGDVGGSALLFSRDHAECLLERKQITRLERGAWPAAL |
| | | PVWFKLSLDIGAEHKALLKQRFKWGVWLNSALVTRNAKDAKGVPP |
| | | PVGTRVLAVDLGLRSAATVSVWQVVDAATPVVAGKWRVPLSDTLS |
| | | AVHERSAMLALPGEHVDAGVLAARRAANEKLAGLLAATSHLSTVFK |
| | | LGRAEQGDRRRELLERLGEGDDRRARAAVATTAAERDGLRAVLGA |
| | | TQDAWAGAVAAVWRRLETDLAGAIAAYRKQQREDVQLRREARHG |
| | | PGASQLPKQAAAERLLGGKSAWQIEYKERVRKLLTRWIMRQRPGD |
| | | TAVRRLARKDLGKYCGGLLDHLTALKEDRAKTTADLIVQAARGRVR |
| | | AHKDAHGRQQDRELWLAKYAPCDLIVMEDLGRYRFATDRPPSENR |
| | | QLMQWTHREVFRLVQMQAEVEGIQVLETGAEFSSKFDARTWAPG |
| | | VRCEPITKLWVERYRNGEMPWLADKADEWRREGIELAQLVPGQLL |
| | | PTGSGEQFVAVSATGGLRVRHADLNAAQCIALRALTGHGTAFRLTA |
| | | RRLGDVFVSAKGLGKRPQGALWREFGSALPPAVVVLRPAGEVRYAL |
| | | RPFASARDAAAALGLQLGALRNVDATDAESDAEDGDLAELLAGAD |
| | | PDRATFFRDPSGDVHGGAWVQAKVFWAEVRRHVRLGLQAQGLLP |
| | | AAARSSEPRQMQLPLAGALPGDDIPL |
| 104 | Cas12 | MLDKFASLYPVTKTLRFRLLPQGRTEENMovAKVLENDLERSEAAA |
| | | WKGLIKKYHLQFISDTLSGSTLSWQALTETLDKFKADHTATAELDSA |
| | | LAAYRCKLAELFTKSPKYKVMATPVSIIKEILKTETDPENIAALNKLNG |
| | | YTYIIFDYVSTRMLTYSADAKATSLAYRLVDENYLRFYQD1SAAAEISA |
| | | VLEEAGFDNAEVEAFIRTDYNTCLTSEGIASFNAAAGSINQFVNVLL |
| | | QQNPVLQSEPALRRHLQPLYKMLLDEAESKIIKFEDYGQLRDAVENF |
| | | RRNFQDLPQSLIDIFAGRYDYSKIYVGYKYLNEASSQIAGGYNWKLLE |
| | | NALEDFYSKPYLVNGKLPVKYKTVVNKKMNQLAYSFTELQEALDAG |
| | | DSGSSITDLFGKYAELHAAYAAADGNVFYKEYDRKSIASLKNYLDAV |
| | | NAIARFIKIFAAPEVYVKDEGFYGIVDGAADKLRDFDLLYNMVRNYIT |
| | | KKPYKKSKVALTFNSSSFGRGWDENKIYDELTTIFTYNGKYYLGVINK |
| | | NDKPDLAAAVSKDEGGYKRMVYKTFDIVKQLPRLSFTKAVKAHFAE |
| | | SDEDFIFDGPKFAKPLRVPKEIYLQSFTDNGDKLADSAKKYTKAYLD |
| | | MSGDYKGYYEAIIKRIDYTKEFLSAYKSTSIYDLAFLKPAGKAAGSLCW |
| | | TRHI |
| 105 | Cas12 | MKTFENFTNLYSLPRTLRFELKPLYKTKELIDSKQELFPKDKRIDEIYQN |
| | | IIKPCLNELHSDFIEKSMENKDFQNIPDNILKIYSNEKNIDDFKNIEKDL |
| | | IKQINWFLKSNKTFFAENYSDLLGKNSIDIIIKVFWEKIYKKDDSWKIF |
| | | LYNDLLWKSYEELINIYFKWFSTYLSNFNKNRENLYDKKNEAKVWSV |
| | | SGRTIWENFPRFLQNCINFRDKLEKLNLSIEQKDIFITNNFWKCISQK |
| | | QIDYYNKIIWQINSKTNEFNQKNGLKWNKKLPKLLLLHKQILWKSEN |
| | | ENILNFINNIIQ.TDFELEQ.EIKIINKDIFQ.RIDFIKKSIVSNIEDFELEKIFIK |
| | | KNRLKDISSLLMDNYSVLEKLLPEFNEEWKIIKENELVNLSKIKKSFENI |
| | | DLKDLKNIFKKEYFDESKDWFKLFLNWIYNHFSDLENNIKHTHKLVQ |
| | | DKLISWNFSENIQKSEKNINLRDEIFVSSKWLLKAYLDSILALDRFVH |
| | | MFDYWEQKDFDSNFYNNIEEYSINFSPFKTYNAVRNYLTKKNYSTD |
| | | KIKLNFDYPDFLGSNSLWKYAFIYKDSKWFYYLWVLDHSNSQSKYKP |
| | | QILKNNTEFYQLEYKQIKFNTLAWKWYIRDFWVKYSEDENCIINLKTL |
| | | IKKQYLERYPVLKEIVDFQTDDKKIFDAKVKTILEQAYSINFVNIDKNYI |
| | | LEENNNWNLHFFQIYNKDFSENKKINSMENLHTMYFKALFEKENFN |
| | | WWACFKLNSQWAEIFFREKSINEKKVKDLKTRNENAIEKKRYTENK |
| | | VFLHLPITLNFINKWYSKYSFWYINDSVKKYIKENKISIIWIDRWEKNLI |
| | | YFSMINENLEIIELKSLNSLILKVSDLEEKEVNYFEKLSKKEWNRNKER |
| | | KDWDEIETIKELKEWYISQIVDNLVKLIVKHNAIVVMEDLNSGFKRW |
| | | RQKIEKQIYQKFELALAKKLNFTVDKNKKHDELWWIYKAYQLTPQIE |
| | | NFQDIYSQTWIIFYTQAAYTSVTCPNCSFRKNIYQKYENESKFKEFFK |
| | | KYILEIKFEDNCFIIKYKIDEKIDKKKNKLKKLEFQVNTKNQIRLKFEKSL |
| | | KWKWWETKEFNITEKFKEIFEKHKLDLWNLKEELLAWNWEIQLYKD |
| | | FMFYFNLLLQLRNSKENDNWWYISCPSCWFHSGNWFQWFSYNW |
| | | DANWAYNIARKWRIILDKIKKDEKNLWITNVEWDNYYQK |
| 106 | Cas12 | MSFLVPHLPTVVSHRLGGYSSAMDQTPTRLTPAVKNTAAATPKPEV |
| | | PLTQRAYTLRLRGANDGDQSWREAVWATHEAVNMGAKVFGDWL |
| | | LTLRGGLDRELADAKVKAGNNNPDRNPTPEERRGRRVLLALSWLSV |
| | | ESAPKKGDAYEKFVIASGKKDSQSIRDEKVVRALREILAKRGVANQD |
| | | LEGWIVDCEPSLSAAIRDDAVWVNRSAAFDAAQCRVGTSLTREEIW |
| | | DLLKPFFGSCESYLASLTTDEDSDTEAAATDDKAKDLVQKAGQWLSS |
| | | RFGTGKGADFAAMSKVYSETAFWAGRASPFRSGAEALRLIAESLKSF |
| | | CPKSFDADGILGLISGPGYKSATRNIIKAWSKRAGPVTADDLANLSA |
| | | VAAEDANKCSANTGSKGHRPWSDAILCEVENACGFTYLQPDGPAL |
| | | HSEFAVMLDHAARRVSIGHSWIKRAEAERDRFTKDALRIKEVPDPIR |
| | | VCLDRFCADRAGTSGAIDGYRIRKRAVSAWKEVITRWGQAACKTAE |
| | | DRVIAVRETQADPDIDKFGDIQLFEALAVDDAECVWRVNGEVTPQP |
| | | LIDYAAATDAEAKQKRFKVPAYRHPDPLSHPVFCDFGNSRWKIRFA |
| | | AHDAVTKLANARTSLDRREADLAKAKERLDKATAPEEQTEGKESLEE |
| | | AERHLRDARDRVAWLSSMHAFSMRLWGAGRVGDGQRLFWSCKR |
| | | LTDDMALRQNSGQAPTIAVTRADRLGRAAAGADTADSVDILGVFT |
| | | EEHWNGRLQAPRAQLDVIAAHVAKNQWDAKAKKMRDRIRWLVS |
| | | FSAKLQPVGPWIEYSATFPEAALAKPFVSRKGEYAVRHVSNDDRAG |
| | | LGKLVLSRLPGLRVLSVDLGHRYAAACAVWETVSIAQTNAACDAAG |
| | | HELPTESDLFLQLSTTDLTGKNRTTIYRRIGADMITDPKTGEKTPHPA |
| | | PWARLERQFLVKLPGEDIPARKASPAEFDAIRRLEEAFGRTRTADDP |
| | | LLVRVDELLAATVDSARLALRRHGDAARIAYAFKPSAEKLTPGGGRE |
| | | VMSPEARKAMILDALLLWHGLWHGDRWADVWASQQWDAYIKP |
| | | ELGMDLPPWSETSGEPRCQYRSKVEGLLKRVAESLAARDGAGLHLL |
| | | WAEQWRTRNAKWLGNTGHLRTLRSLLLPRGLTTSTPAAWNVGGL |
| | | SLTRIATLKSLYQLHKAYHMRPEPEDPRKNVPAKGEEELRDFGRGML |
| | | DVMERLREQRVKQLASRLAEAALGIGRMKASEGKRDRKRPRAQID |
| | | QPCHAVVIENLKNYRPEETRTRRENRQLMSWSSSKVKKYLSEACQL |
| | | NGLHLREVQASYTSRQDSRTGAPGIRCADVPVQDFFTKPWWRRQ |
| | | VSIAVGRVNQGRGDARERFLADLDAKWSAAEKSERITAPPLRIPVN |
| | | GGELFVSADLHSPAALGLQADLNAAANIGLKALLDPDWPGKWWF |
| | | VPASLDADGWRVPAAKSCAGAEWVKNWKVGQLGDSYAPNGKPL |
| | | QPTDDEGVKKAEDGVKLAKASLDDAEQALKAAKKTKRKAEIDVASA |
| | | RAQEAKKNVDDMKKALVAAKKGASAKEIINLWRDPVGVDPADFAS |
| | | GEPWRAYTVYKQRVEYHVINDVLRGRAGPRSDRP |
| 107 | Cas12 | MSITRSIKVKLIVPRDASLEARQLREGLWATHLFVNDGCHYYERLLLE |
| | | FRQRDVCVGKDDAGKDVIVPAAEWADRLRARLGRNGMVPSHIEA |
| | | ALPIFRELYENMVPSALKAKSGTGQAGRSWHSKLVSPTSRGGEASA |
| | | ARIDVLRPLLPVSGDDPAFEPAARALIEEAGDELLTSTGRCPAWVTA |
| | | YRKGPEGSAWVEKLRIQLREAVEAGDFDPPSDPQILAAGAVPAAPP |
| | | LGAGIDALRPLLPLLGGDPAFEPAARALVEDIGDELFTSTGRPPTWV |
| | | TAHPTWVRAHRKDAECLEAADDFKWVERLRQRLRDDAKAGKFEQ |
| | | PLHERLGALGALPVAKPIGAGRVVSRADLTVFERGAMELAIEHLIGW |
| | | ESAGHRARAQYVERKKRHDDLLQWIEAEAPDALLAVRAYEAARTIH |
| | | LATLGELGAAPQYTLRLREIRPWRKLREWLLQNPDATIDERRRRLAT |
| | | MQTNDPRGYGGEALAWLAAPERRALVEHPAGDVVTRIAVLNIRKSI |
| | | LDRSRLFPTCTLADPVEHPRFAKFGKPGDKNSAGYALAVDGVRREAI |
| | | IKILVPRQDGLLVPTDLRVPFAPSGQMRDLRASGLDISYERQDGRGR |
| | | QAAKLQGGNLMFDRTHFARCGAPGPEALGSVWIKVALDLSSPAAS |
| | | LAMKTATPVRTYLSTAVRGRPESTKYEKAAPPEGFRVLSVHMGLRT |
| | | AATASMLRFGAPEEGGHEVPVSGLAGETLVAFHERTVTMKLPGED |
| | | PDTRTEANRGVAKRELRGLGRGIGCLKAIRRASASATPEDRAEALVII |
| | | ETHVGQGDRHGWAPAEAVGRLDPHGDPDDWKTACAALYAAVEA |
| | | DLGVAISSWRKAARAGGATGMLGGKSLWAVDHLERSFRFLRSWDL |
| | | RARPHDGDPRRPRPGYASKLLHHIDGVKDDRVKTTADRIVQAACG |
| | | RAWIGGPTVKRGTQDVRLPGRWEQRGPRADLILLPDLTHFRFRSDR |
| | | PRAENSRLMRWAHRQLAIYVRMQAEVEGILVADTGAAFTTRFDA |
| | | WTGAPGVRCEPVTADHLRGIAKREDYWLARLLREGALKHLRIDPAS |
| | | LRVDDLVPMDHGKILVALDGVDLPGLRILDTDVNASQGLGRRYIEG |
| | | HGLAYRLPGARVPRGEGEREAAVVHIKGKRLASAMGGTVVVLRASE |
| | | GPGDITWTAEVYDRPQGARKALGLSLAAFNSIATAAVDDEGPAPEN |
| | | DDEALEEEAEEALGIATGERIVFFRDPSGAVAGGGWLEASAFWGIA |
| | | NRMVTDRLRELGRLG |
| 108 | Cas12 | MATAVDTSTTRAYTLRLSGGNNWRELLWQ.THVAVNRGAWVWG |
| | | DWLLTLRGGLPASLADGDAERRVVLALSWLSVESPASLAPQAHIVA |
| | | YGSDARDERNRKVTERFRDILRRMGIKQQQEQEWLDACLPALMAS |
| | | IREDAVWVDRSACFAEAQQCYRGLSSEWARKTLFDFLGGEDDYFKP |
| | | SAKEGASSKAKDFVQKAGRWLSRHWGAGKKSDPRDISTRLGKLAG |
| | | VDPKAIDGHTGRAALEDLLRTLGSRPAQNADAEKLYRQLKRAVGW |
| | | KGRPSKGAVALKKIRDAERVPNDLWKEIASTLREEAAVQSSQTSDH |
| | | AAVPDWRSHWPAEITGLPMPYRVDRDYIWEHGVMLDHALRRVSS |
| | | AHTWIKRAEAERRRFQQDAAKMGSIPEEARNWLDAFRERRSSSSG |
| | | ATGDYLIRERAINGWDKVVQAWETLGPNSTRDQRIAAARDVQANL |
| | | DEDEKFGDIQLFAGFGDEHVDDPERCLADDRATCVWRNSSGRADG |
| | | RILKDYVAATVAEHNQRRFKVPAYRHPDPLRHPVFVDYGKSRWSIN |
| | | YSALTAAQQRRKTTQKLAQAKTDNTRAKLQQQLASTADLRSVTLGV |
| | | WDGNRIVKISQRWRSKRFWRDLDLDHFGSHPSAAVSRADRLGRVA |
| | | ARQDPGAAVYVAKVFEQQDWNGRLQVPRRELNRLADVVYGKGA |
| | | DPDFGKLERLDPRARRLWERLSWFLTTSATVQPQGPWLDYVAAGL |
| | | PSGIQYTKSRAGYYLNYDANHGRKGRARLCLARLPGLRVLSLDLGHR |
| | | YAAACAVWQTLTIEQMTNECRQAAHPAPSNDDLFIHLRHPTHKPQ |
| | | KSGRKKGRPVTKTTIYRRIGPDKLPDGTDHPAPWARLERQFLIKLQG |
| | | EDRPARYASQKEIDEVNQFRNFVGLEPIVDRPRVDDLHSDAVRVAR |
| | | LGLRRLADAARIAFAMTAAKKPISGGHEVELTTAQRIEFLQDALLLW |
| | | QSLAASRRYRDDWAEKLWQSWVVEKLGGPQPAEIADDLPRSQRA |
| | | ASLKTARQSLRKVAEKLSDGQSPSAAELHRLWAERWQQRQTEWR |
| | | RHLRWLRRLILPRRKDHQQEDRPLQRVGGLSVKRIQTIRQLYQVLKA |
| | | FRMRPEPSDLRKNIPAPGDRSLASFGRRILNHLERLREQRIKQLASRV |
| | | VEAALGAGRISKPPGRDRRRPQQPVDRPCHAVVIENLQHYKPEDSR |
| | | LRRENRQLMDW/QARNLRKYIVEGCELHGLLFVEVSPAYTSRQDSRT |
| | | GAPGLRCEDVSRTALQEAARRMHASHSRPSNSSPGGSQTQFEREV |
| | | CRWINEFKRVEGSSSSLSARQAVLKAFLHHQASIPTSLSTILLPRRGGE |
| | | LFVSADPDSPLACGLQADLNAAANIGLKALTDPDWMGAWWFVLV |
| | | DRASGQPVEEQVQGCPIWLSCGPLSNSNPATIDPSDSPTAARRSNG |
| | | TGAKGRARANEYWWSSLSATTLPDHKAWQPTQDYWRDIEQRVV |
| | | KRLLRLLDGSEWSED |
| 109 | Cas12 | MLDKFASLYPVTKTLRFRLLPQGRTEENMQVAKVLENDLERSEAAA |
| | | VVKGLIKKYHLQFISDTLSGSTLSWQALTETLDKFKADHTATAELDSA |
| | | LAAYRCKLAELFTKSPKYKVMATPVSIIKEILKTETDPENIAALNKLNG |
| | | YTYIIFDYVSTRMLTYSADAKATSLAYRLVDENYLRFYQD1SAAAEISA |
| | | VLEEAGFDNAEVEAFIRTDYNTCLTSEGIASFNAAAGSINQFVNVLL |
| | | QQNPVLQSEPALRRHLQPLYKMLLDEAESKIIKFEDYGQLRDAVENF |
| | | RRNFQDLPQSLIDIFAGRYDYSKIYVGYKYLNEASSQIAGGYNWKLLE |
| | | NALEDFYSKPYLVNGKLPVKYKTVVNKKMNQLAYSFTELQEALDAG |
| | | DSGSSITDLFGKYAELHAAYAAADGNVFYKEYDRKSIASLKNYLDAV |
| | | NAIARFIKIFAAPEVYVKDEGFYGIVDGAADKLRDFDLLYNMVRNYIT |
| | | KKPYKKSKVALTFNSSSFGRGWDENKIYDELTTIFTYNGKYYLGVINK |
| | | NDKPDLAAAVSKDEGGYKRMVYKTFDIVKQLPRLSFTKAVKAHFAE |
| | | SDEDFIFDGPKFAKPLRVPKEIYLQSFTDNGDKLADSAKKYTKAYLD |
| | | MSGDYKGYYEAIIKRIDYTKEFLSAYKSTSIYDLAFLKPAGKAAGSLCW |
| | | TRHI |
| 110 | Cas12 | MCIRDRDLAVAALNRGDAXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXGQLNAIRNFLASDGYGAHDLVPVNYNGSNDRGAEVQVGN |
| | | QKNWTVTSSAPVVVQYNNADTTLTVQGHTDRLIVTGSGNDTITLK |
| | | DSGDDKVLLGDGNNTVVAGSGADTIVGGAGNDVLIGTNGGYGTEL |
| | | LGGAGNDVLRNAGSHGIYMDGGAGNDTFYGGTGPDTMEGGDG |
| | | NDLMYANGRGSSIDGGAGNDTIYGGPGGDTLTGGDGNDLLRSDS |
| | | AFGTKGSGNLLVGGAGNDTLWAGAGYDTLKAGSGSDTLISGTGSS |
| | | QMIGGSSGNTTFEVAYHTGNDTITGSGSGNTVYLDGRDFSDATISN |
| | | HSGVTTVSFSDGQVLKISGVQDIVFSDHDYKV |
| 111 | Cas12 | MSITRSIKVKLIVPRDASLEARQLREGLWATHLFVNDGCHYYERLLLE |
| | | FRQRDVCVGKDDAGKDVIVPAAEWADRLRARLGRNGMVPSHIEA |
| | | ALPIFRELYENMVPSALKAKSGTGQAGRSWHSKLVSPTSRGGEASA |
| | | ARIDVLRPLLPVSGDDPAFEPAARALIEEAGDELLTSTGRCPAWVTA |
| | | YRKGPEGSAWVEKLRIQLREAVEAGDFDPPSDPQILAAGAVPAAPP |
| | | LGAGIDALRPLLPLLGGDPAFEPAARALVEDIGDELFTSTGRPPTWV |
| | | TAHPTWVRAHRKDAECLEAADDFKWVERLRQRLRDDAKAGKFEQ |
| | | PLHERLGALGALPVAKPIGAGRVVSRADLTVFERGAMELAIEHLIGW |
| | | ESAGHRARAQYVERKKRHDDLLQWIEAEAPDALLAVRAYEAARTIH |
| | | LATLGELGAAPQYTLRLREIRPWRKLREWLLQNPDATIDERRRRLAT |
| | | MQTNDPRGYGGEALAWLAAPERRALVEHPAGDVVTRIAVLNIRKSI |
| | | LDRSRLFPTCTLADPVEHPRFAKFGKPGDKNSAGYALAVDGVRREAI |
| | | IKILVPRQDGLLVPTDLRVPFAPSGQMRDLRASGLDISYERQDGRGR |
| | | QAAKLQGGNLMFDRTHFARCGAPGPEALGSVWIKVALDLSSPAAS |
| | | LAMKTATPVRTYLSTAVRGRPESTKYEKAAPPEGFRVLSVHMGLRT |
| | | AATASMLRFGAPEEGGHEVPVSGLAGETLVAFHERTVTMKLPGED |
| | | PDTRTEANRGVAKRELRGLGRGIGCLKAIRRASASATPEDRAEALVII |
| | | ETHVGQGDRHGWAPAEAVGRLDPHGDPDDWKTACAALYAAVEA |
| | | DLGVAISSWRKAARAGGATGMLGGKSLWAVDHLERSFRFLRSWDL |
| | | RARPHDGDPRRPRPGYASKLLHHIDGVKDDRVKTTADRIVQAACG |
| | | RAWIGGPTVKRGTQDVRLPGRWEQRGPRADLILLPDLTHFRFRSDR |
| | | PRAENSRLMRWAHRQLAIYVRMQAEVEGILVADTGAAFTTRFDA |
| | | WTGAPGVRCEPVTADHLRGIAKREDYWLARLLREGALKHLRIDPAS |
| | | LRVDDLVPMDHGKILVALDGVDLPGLRILDTDVNASQGLGRRYIEG |
| | | HGLAYRLPGARVPRGEGEREAAVVHIKGKRLASAMGGTVVVLRASE |
| | | GPGDITWTAEVYDRPQGARKALGLSLAAFNSIATAAVDDEGPAPEN |
| | | DDEALEEEAEEALGIATGERIVFFRDPSGAVAGGGWLEASAFWGIA |
| | | NRMVTDRLRELGRLG |
| 112 | Cas12 | MAQASSTPAVSPRPRPRYREERTLVRKLLPRPGQSKQEFRENVKKLR |
| | | KAFLQFNADVSGVCQWAIQFRPRYGKPAEPTETFWKFFLEPETSLP |
| | | PNDSRSPEFRRLQAFEAAAGINGAAALDDPAFTNELRDSILAVASRP |
| | | KTKEAQRLFSRLKDYQPAHRMILAKVAAEWIESRYRRAHQNWERN |
| | | YEEWKKEKQEWEQNHPELTPEIREAFNQIFQQLEVKEKRVRICPAA |
| | | RLLQNKDNCQYAGKNKHSVLCNQFNEFKKNHLQGKAIKFFYKDAEK |
| | | YLRCGLQSLKPNVQGPFREDWNKYLRYMNLKEETLRGKNGGRLPH |
| | | CKNLGQECEFNPHTALCKQYQQQLSSRPDLVQHDELYRKWRREYW |
| | | REPRKPVFRYPSVKRHSIAKIFGENYFQADFKNSVVGLRLDSMPAGQ |
| | | YLEFAFAPWPRNYRPQPGETEISSVHLHFVGTRPRIGFRFRVPHKRS |
| | | RFDCTQEELDELRSRTFPRKAQDQKFLEAARKRLLETFPGNAEQELR |
| | | LLAVDLGTDSARAAFFIGKTFQQAFPLKIVKIEKLYEQWPNQKQAGD |
| | | RRDASSKQPRPGLSRDHVGRHLQKMRAQASEIAQKRQELTGTPAP |
| | | EIIIDQAAKKATLQPFDLRGLTVHTARMIRDWARLNARQIIQLAEE |
| | | NQVDLIVLESLRGFRPPGYENLDQEKKRRVAFFAHGRIRRKVTEKAV |
| | | ERGMRVVTVPYLASSKVCAECRKKQKDNKQWEKNKKRGLFKCEGC |
| | | GSQAQVDENAARVLGRVFWGEIELPTAIP |
| 113 | Cas12 | MNIIMENFEKFVNLYELSKTLRFELIPFSQTKVELEKDWIIEKDREIEEK |
| | | YHIIKEKLDTLH1KFVWQALEWVDLSLLEEYAELYFACKKDTKNKKLKS |
| | | KFEKLEKKIRQEITSFFDAEWNKWKEKYGFLKKWWTSFLTEKEILDVL |
| | | IDIFPENKDDFEIFKWFFTYFSNFNESRKNFYKDEWKAGQIATRAIDE |
| | | NLTTFLENIIKYKNFKKENPDFFTENEEKVFELDFYNFCLTQKWIDNY |
| | | NEIIWAKSLEEWKNTQGVNQRINLLKQKNEKSNKKNLSYPKFDILYK |
| | | QILSEKSENDFIPNIENTEELFTVIQKSIKENDKKITEIDKLFKKFFLEEN |
| | | NIDIWKVYISKQAVNTISSKYFENWSSLWWYLWENSKKKYFSLWEI |
| | | KEALEDIKEKNIFKGEYYNNKIAFENKSNFENFLAIFYYEFQTNLSLLN |
| | | WNQNNLESLQEKEFKKEEKQVDIIKKYFDSVMDLYAMSKYFFVDLK |
| | | QAKNFPKDIEFYNDFDLYFSDYEPWKVYNLVRNFLTKKEVKTDKFKL |
| | | NFSNSQFLTGWDKDKEKERFWVILRKNEKYFLAILKKNNNKIFENYR |
| | | ENNPTDFYEKMEYKQLNNVYRQIPRLGFPLQKKLDSLKWKELEEYLE |
| | | KYKNNFWYNKEIAFIKEEFDIFQKNKEKWEKFDREKLKKLIDYYKKVV |
| | | LEKYSDLYDLKKLENTDYDELVNFYDDVEKSMYSLHFTKIETEFLENLE |
| | | KNWEIYLFQIYNKDFSDYKKENTKENIHTKYFKHLFSEENLENLKIKLS |
| | | GWAEIFFRDKTHNLKQKLDKNWKKMFYWENKDKKVLEHRRYAKD |
| | | SYGFHISITLWANNWDMYKFNQFFNKNFTPKHIIGIDRWEKHLAYY |
| | | SVIDLEGNLVETDTLNIVNGINYLEKLENIEKSRMQERKSWWEIENIK |
| | | NLKDGYISAVVSKLTELIEKYQAIIVFEDLNLGFKRWREKIERQVYQKL |
| | | ELALAKKLNYLTFKNKKDCEIWGVLNGIQLVPRVKDYQDIANYKQSG |
| | | IIFYTNPAYTSTTCPECGWRKTLKFPSKITKTSILEFFKEIQMSFYWEKF |
| | | SFTYENILWKSETLFSNVKRTQWNNKSRKIEHKENITQELKSVFEKYN |
| | | INLWENISEKLQQTDMLLEDLKVLFYNFKLINNIRNSDSKLEEDIISCP |
| | | CCLFNSENWFKGAYFNGDANGAYNTARKGIIMLENIKVNPERSNLF |
| | | VRNEEWDEFLREEF |
| 114 | Cas12 | MSKLFENMTNLYSLNKTLRFELKPVGNTRELIESKDFFKNDEEKAEN |
| | | YQFMKEKMDKIHRNYIQKSLETIKMLPILDQTEQKRLKKEDIKNELKS |
| | | LRSFISAAFVSVKDLLSNKIIEWLISEASIEEKEKIKKFDKFFGYFKTYVQ |
| | | NRGNLYKAEDKAGQIAFRLIDENLPRFFKAKQIIEEIIKKTPDFVVKER |
| | | NEKQEETEKKITEYLSIFYLNSYCHYLSQSGIDLFNEIVGHINLSINLYK |
| | | QKTGVKFSLIPLLYKLPLAPRKQISRLPKQIENPEELELIVKSVLDRIDQ |
| | | KINPFINNILQTVLEENSVYDLNWIYISIKTTEQWWSIGLSGRNSLREL |
| | | GYNQSKNWEEPVLKAEKNKFPFISLGEIKSFLDAKQLAWDDIKSVF |
| | | WEGKSQSLMNWSWSNIFFHLIGKEISSLIEKYLHSRKNYQISQSKEN |
| | | QKHVLDNSLSLWRLLGWFVLMHKKTHLTPEIKESFFYDWENGLDAI |
| | | VFDENSDPIHTIYDKVRNCLTKKPYSNKDKIKVNFDCPYLLWGWDQ |
| | | NYDAFGWLIFHDWKKYFLWLIKGSWLNMEEKNKLYEWINPMNSIT |
| | | KHYDYQKPDFKNVPRLFIRSKWDTFAPMVREYWLPVNDILYLYDNE |
| | | LYKPDKKNPWKHKSYLRRLIDYFKLWLSKHKSFKHYIFKRKESDQYEN |
| | | LAEFYSDVEFSCYAIKKEKVNFDQVKALCESERLYLFEIYNKDYNQFSK |
| | | WKNKNLHTQYFEALFEERDNNLFMLSGGWSIFWRESNWKIVEKIR |
| | | SYSPKYNIEIIDKRRYTKNKLMIHIPIVLNFCRNQEWRVNDMIKSLIQS |
| | | QSNNFTILGIDRWEKHLLYYSLIRQDWTIIKTGSRNTITNKIKIVDYHK |
| | | KLDDREKKRDEAQANWEQQEQIKDLKKWYISQVINEISKMIIEHNAI |
| | | IVLEDLNGWFKRWRQKVEKSIYQQFELALAKKLNHLVFKNRWDTES |
| | | WGTMKAYQLTPLVAQFQDLSFQTWVVFYTPAWYTSTTCPCCGWR |
| | | KNIYFKYENEKQAKIELEKLNIVRENNYFSITYTAEWWNKKWKITWS |
| | | VLNKTDRILTTKWQTRLQYDRASKNTKEYDITTDFNSVFTAKFLDYKI |
| | | RLENAGSKQCRNLINSINLLLKIRNAKSGTDIDTIQCPACEFHSQWW |
| | | FQWNEFNWDANWAYNIARKGKVIIDKIVKGEKNTTVSQIEFDNEIQ |
| | | K |
| 115 | Cas12 | PAYRHPDPLLHPVFCDFGCSRWQICFDVRKNVRTTSPRRLCLTLFTG |
| | | SGMELVPFSWQSKRLARDLALEQRHRNPEASEVTRADRLGRAAAG |
| | | VSAGGAVSIKNIFNEENWNGRLQAPRSQLAAIAARVDKHGWDAK |
| | | ALRMLDRLRWLITFSPRLEPTGPWIEYALRIPDDAAAKPFLSRKNGF |
| | | AVLHRSNDDRKGLAKLILSRLPGLRVLSVDLGHRYAAACAVWETLSA |
| | | DQVREACQAAGHDGPTECDLYLHLKKDGRTVIYRRIGADTLSDGTP |
| | | HPAPWARLDRQFLIKLQGEEHKAREASNAEIWAVHQMEAALGRSV |
| | | PLIDRLVASGWGQRTEGQRARLEALKQLGWRPVAEKLDSDDEPGV |
| | | MEESPAIKPSLSIDELMSSAVRTMRLALKRHGDRARIAHYLITDEKTK |
| | | PGNVKEQLDENGRIELLQDALVLWHDLFSSKGWQDNKAKELWYV |
| | | HIATLPEYKAFQSSGEGHAGPERRGKPEEDREKLGVVAQALAPNVT |
| | | LREALHNAWKKRWEEDDARWKALLRWCKDWILPRGEAANSPAIR |
| | | KVGGLSLIRLATLTEFRRKVQVGFFARLRPDGKKAETGEKFGRKTLDA |
| | | LEELREQRVKQLASRIAEAALGIGKEHVFPTAKEPQKKCWCGSVHRK |
| | | KDPKRPRERVHSPCHAVVIENLTHYRPEETRTRRENRQLMSWSSGK |
| | | VKKYLAEACQLNGLHLREVNAAYTSRQDSRTGAPGIRCQDVPVKEF |
| | | MRSPFWREQVAQAKKKQAEGKGDARERFLCDLDEQWKNRSEDE |
| | | WKKAGPVRVPLRGGEIFVSADGTSPTAKGLHADLNAAANIGLCALT |
| | | DPDWPGRWWYVPCDPASFKPIMDKVEGSAAVQPDQALRQPAQA |
| | | QRGDAARDRKKRGKVGGRSREVVNLWRDVSSRPISPNDSWQEFT |
| | | PYWNDVQARVVNILRQSAGLTRGQGEP |
| 116 | Cas12 | MPLRSINIKMRLKRAAEGRALRQSLWLTHSVVNCAVAEIERVLLLCR |
| | | GRGYWTGDDEPVSAETVQQQALAFARETQARNGQPGIGGDAEIL |
| | | AALRGLYEAIVPSVNRDEQGRPLEGNAQAAGGFAGPMMDAESEG |
| | | FQSVFDKILEPLPSWVAKMTGGTRGWEQESVQWLKSPESERLQHA |
| | | SGSPPAWVRRLRTGEPWQEAFVQDQENKRKEVKGVPSLIQRLKKE |
| | | LGLLPLMRPPITLQFEDKRSGLTPWDRLTLRLAVAHLLSWESWNHR |
| | | AADEHGRVTERLARLETEAAPLASLIEGLREYEKIRHEELKRVAQAND |
| | | ENPFRIGARGVRGWDRVREAWLGATDDTKEGRFTSVAALQ.TKLGG |
| | | RFGDPDLFRWLAEEGREHLWGEHDPLPILARLNALSRLLRRKKDHAI |
| | | YTAPDARLHPRWTAYEAPGGGNLRNYSFEISGNDLALRLPLLRRVES |
| | | GLEEDSQRAEIALAPSGQFQSASWKGDGKPNRHLTYYSAHEQFSAE |
| | | LGGAEILYRRRHLENRKVKELEQGDIGPVWLKLVLDVQPNAPEGWF |
| | | TPRGRVVTPPTVHHFNTALVNRSKHAPDLVPGLRVLAVDLGVRTFA |
| | | ACSVFELVQGEPGNGMAFLADQERDLWARHERSFLLPLPGEAVDS |
| | | QLLAARRAAYNQLGLLRRDLGRLKGILRLSVKETAENRCGSLEELLAS |
| | | LEDEWNRGNAPAIDVAALLSAKGCLEMPQPAWEAAIAGTYQAAER |
| | | ELGNRVRDWRRTTRPRTTGEDDRRQRRGYSGGKSAWAVEYLDQV |
| | | RRLLQGWSLHGRSYAQIRRLDREKMGTFAAGLLDHINALKQDRVKT |
| | | GSDLIVQAARGYLPTGKKGWIKQYEPCRLILFEDLARYRFRTDRPRRE |
| | | NARLMRWNHRQILTETELQAEIFGLLIGTTGAGFSSRFHARSGAPGC |
| | | RTRLLTAEDLRSPTLAKQLELLAETQGIDPKCFRPGMQIPWDSGADF |
| | | VTLDSEGKLVQIHADVNAAQNLQRRFWTRFRDAYRISAVEIKQDGR |
| | | TSWYPDREGVRLRGALSTIVGGDGYARLVAADDDDGFVLEKVTRP |
| | | HWRRAAGAQAESGDDVGLDEVALEMAEAMDTDLERGEGRLVFFR |
| | | DPSGSVIRADRWYQAKAFWGQVRSRVTRALGLRRPGASS |
| 117 | Cas12 | MLDKFASLYPVTKTLRFRLLPQGRTEENMQVAKVLENDLERSEAAA |
| | | VVKGLIKKYHLQFISDTLSGSTLSWQALTETLDKFKADHTATAELDSA |
| | | LAAYRCKLAELFTKSPKYKVMATPVSIIKEILKTETDPENIAALNKLNG |
| | | YTYIIFDYVSTRMLTYSADAKATSLAYRLVDENYLRFYQD1SAAAEISA |
| | | VLEEAGFDNAEVEAFIRTDYNTCLTSEGIASFNAAAGSINQFVNVLL |
| | | QQNPVLQSEPALRRHLQPLYKMLLDEAESKIIKFEDYGQLRDAVENF |
| | | RRNFQDLPQSLIDIFAGRYDYSKIYVGYKYLNEASSQIAGGYNWKLLE |
| | | NALEDFYSKPYLVNGKLPVKYKTVVNKKMNQLAYSFTELQEALDAG |
| | | DSGSSITDLFGKYAELHAAYAAADGNVFYKEYDRKSIASLKNYLDAV |
| | | NAIARFIKIFAAPEVYVKDEGFYGIVDGAADKLRDFDLLYNMVRNYIT |
| | | KKPYKKSKVALTFNSSSFGRGWDENKIYDELTTIFTYNGKYYLGVINK |
| | | NDKPDLAAAVSKDEGGYKRMVYKTFDIVKQLPRLSFTKAVKAHFAE |
| | | SDEDFIFDGPKFAKPLRVPKEIYLQSFTDNGDKLADSAKKYTKAYLD |
| | | MSGDYKGYYEAIIKRIDYTKEFLSAYKSTSIYDLAFLKPAGKAAGSLCW |
| | | TRHI |
| 118 | Cas12 | MSRSIFSPFTNLYPIQKTLRRELKPLNENFQHDPALSALRNSEIPQRD |
| | | EQREKDYQAIKPLLDEFHNQFITESLHSLEPQDRSDFILFYQTYQKKK |
| | | KNKAEISEKELKSLDEEFESRTKALRNAIGTSFSVTAELRKSNPDYVSE |
| | | KGKPFLTQKSYKILTEAGVLWLLEKKYTSDPEKLALIRRFGNFFTYFTG |
| | | FNQNRENYYATDEKSTAVAYRAINENLLTFANNCELFEKLSVLSLSEL |
| | | EKKTFNPDSYSEYLTQSGIVFYNEMLANIRSKANLYTQEHKAKLPQP |
| | | KLLYKQIWSPRGDTIPFDLIASEAEFQETLHTMIRETDQRIPEFNKLLE |
| | | QIFEEKVDLSQIFFSKTSLNIISNRYFSSWHTLLEKGVELKLFKFKKNDE |
| | | ESFKLPAYLSLAELKELLESAPFQMAEKADADEEKHHQASLFKLQRE |
| | | NLHLEKSHSNWELLLKSMKSDFESFWTWEGEFWSYTLAKKALQSLS |
| | | ALESTNQEHKNLIKMLLDNALYAYRMLKWFKVDTSKLGFVPEGEFY |
| | | PSLDQLLQDYPLPKWYDMIRNYLTRKTYSQAKLKLNFDCSTLLNGRD |
| | | KNKEIQNLSVILRKDGKFYLAIMKKDQNKFFENSALYEGNLGTMEK |
| | | MDYKLLPWANKMLPKCLMPGSDKKKYGASDQVLELYAKGSFKKSE |
| | | KSFNLADLHTLIDFYKLALPKYEDWKVFNFQFQATENYQDISQFYRE |
| | | VEQQGYLLNWRKVNEKLIKQGIKDWSLFLFQISSKDFEGKSKTPDLQ |
| | | TLYWQQLFEFSTNVKLNGEAEIFFRPWSMKKEKKKLKVDNYDVFKH |
| | | KRYTEDKILFHVPITLWFGNNEVSPSAPSKFNQKLNQELIIPHFDDLH |
| | | VIGVDRWEKHLAFYSVVSVKTGKIVKQGTLNLLNGTDYEAKLSQKAE |
| | | NRLHARQNRDTIEKIADLKNGYISQVVNKLVELVLEYNAVIVFEDLNA |
| | | GFKRGRQKIEQSVYQKLELALAKKLNFIVKKEKAVGEPWSVTSAYQL |
| | | APQINTFWDIKWKQRGIMLYTRANYTSVTDPLTGWRKQYYFKKGS |
| | | SEEMKAQFFKSFKNLTRDAGQEAYIFDDGTWLLYSNVERRRGKRGD |
| | | HRERTQIKYDPSLELDTLFSKYQJEKSDSLFDQLKNRELPQTFWTSFFR |
| | | IIDLIMQIRNTDDEGRDIILSPIGNPQERFDSRKRYNQLPRDEKGGIIE |
| | | ESAFEYPTSWDANGAYNIARKGVMMLERIKENPEKPDLLIRDAEW |
| | | DKKITRK |
| 119 | Cas12 | MKNFQDFTNLYELSKTLRFELKPIWGTKKLIEEKNILKLDKKKRENYEK |
| | | VKPYFNKIHQEFINFALRNPNFDFSQFEEKYLNWLKDKKNKDLLKEK |
| | | ESIDKIFLEKIWKLFENSVKDFLKENGFESIVKSEDQNLKFFRRKEIFEV |
| | | LQEKYGSELETQMVNKDWEIKSIFNGWEKWLWYFDKFFNTRDNFY |
| | | KTDWTSTAIATRIIKDNLKIFLENTIIFEKVKNKKIDFSEVEKNFSVSIDT |
| | | FFEINNFNNCFLQDWIDFYNKVIWGETLENWEKLKWLNEIINKYRQ |
| | | DTGEKIPYFKKLQKQILSEKDWVFIDKIEDDGGFYEVLKNFYKNAAEK |
| | | EWFLKNIFENFYTISDKNLEKIYFNKIAFNTISHKFWSALEFERILYEEM |
| | | KKEKADWIKFEKKENKYKFPDFIQIIFIKRSLENYDSENLFWKERYYKS |
| | | EENVDWFLEKNNNNIWEQFCKILNFEFLNILKRRIIDEAWEEYEVWF |
| | | EISKNILWEKLENFELNQENKWIIKDFADYSLALYSFWKYFAVEKWR |
| | | NWDLNIDISDDFYGWEDWYIEKFYNTGYDEIVKPYNLMRNYISKKP |
| | | WEDSKKWKINFETSSLLSWWDKNLESNWSYIFQKWNKYYIWIINW |
| | | SKPAKEVLEKLYSWNGEKIKRFIYDFQKPDNKNTPRMFIRSKKDSFSP |
| | | AVGKYNLPVEDILEIYDNWLFKTENKDNSNYKESLSKLIDYFKLGFSK |
| | | HESFKHFNFVWKDSKEYENIADFYRDVEKSCYQITSEFLDFEELKKLT |
| | | FKKHLYLFQIYNKDFELDESLQKNWYNFRDEWQKNIHTKYFEALFLE |
| | | ENILRKSWAVFKLSWGWEVFFRKESIKAEKEKRKNIEVTKNRRYTEE |
| | | KYFLHFPIQVNFKNEISWNFNQEINKFLANNPDINVIWIDRWEKHLA |
| | | YFSVINQKWEILESWSFNKIENYNKNWEKLLFPEREIKEIHKDWSLID |
| | | LELVETWRKVDYVDYKLLLEYKERKRLLQRQSWKEVEQIKDLKKWYI |
| | | SALVRKIADLIIKHNAIVIFEDLNFRFKQIRGWIEKSIYQQLEKALIDKL |
| | | NFLVNKNEINLEKAGSILKAYQLTVPVDSLKEIWKQTWVIFYTEAAYT |
| | | SKIDPIKWWRPNLYLKKQNAEINKENILKFDNIIFNSKENRFEFTYDLK |
| | | KFFWKDSKFPAKTVNTVCSCVERFKWNRNLNNNKWGYIHYENLTD |
| | | WKLANKEQKEDEFSNFKELFEKYFIDINWNILEQIKNLDTKNNEKFFS |
| | | SFIDLFTLVCQIRNTNQNAKWDENDFILSPVEPFFDSRKSQNFWKSL |
| | | PKNWDENWAFNIARKGLIILNRISENPEKPDLLIFNADWDNFARNI |
| 120 | Cas12 | RPGASAALSLTPLKTLFSENFSSFMCGNWQIINDSLKTYYNENIKSKG |
| | | KAKEEKVKKAIKAIEYKSLADINQLVERYNNDELNRKAEEYISAINEKIK |
| | | DLDVNEIEYDEKINLIENETKSEEIKSKLDSIMEIMHWTKMFIIEEEIEK |
| | | DVNFYNEIEEIYDELQPLVTIYNRIRNYVTQKPYSEEKIKLNFGIPTLAN |
| | | GWSKTKEYDNNAIIMIRDGKYYLGIFNAKNKPDKKIMEGHQSEENG |
| | | DYKKMIYRLLPGPNKMLPKVFMSKTGIAEYKPSQYILECYEQNKHIKS |
| | | DKNFDIKFCRDLIDFFKTSINRHPEWSKFNFKFSETSEYEDISTFYREV |
| | | EKQGYKIEWTYISEKEIKELDENGQLYLFQIYNKDFSEKSKGKENLHT |
| | | MYLKNLFSEENLKNIVLKLNGEAEVFFRKSSIKKPIIHKKGSVLVNKTY |
| | | NENGERKSIPEEQYTEIYKYLNSIGTNELSEKSKKLMEEGKVEYYKAN |
| | | YDIVKDYRYSVDKFFIHLPMTINFKAAGFSPINNIALKNIALKDDMHII |
| | | GIDRGERNLIYVSVIDTKGNIVEQRNFNIVNGIDYKEKLKQKELDRDN |
| | | ARKNWKEIGKIKDLKEGYLSLVVHEIAKLVVKYNAIITMEDLNQGFKR |
| | | GRFKVERQVYQKFETMLINKLNYLVDKDLAVDQEGGLLRGYQLTYIP |
| | | ESLKVLGRQCGYIFYVPAAYTSKIDPTTGFVAIFNYKGMTDKDFVTSF |
| | | DSIKYDDERGLFAFEFDYENFVTHKVEMARNKWTVYTYGERIKRKFK |
| | | NGSWDTAEKVDLTYQMRSILEKYEIEYNKGQDILEQIEELDEKAQNG |
| | | ICKEIKYLVKDIVQMRNSLPDNAAEDYDAIISPVINNNGEFFDSTRGD |
| | | EDKPLDADANGAYCIALKGLYEVMQIKKNWNEETEFPRKELKIRHQ |
| | | DWLDFIQNKRYL |
| 121 | Cas12 | MQCGSLRHNSKSLDSALAYPARLTTGGNEGKTSEAHIENDSLPEITR |
| | | RPSRLARDFAPSNPFPFMKRIYQGQITGMQFHGAAGEQAVPANQ |
| | | NWEQALWDHHGLFHDAVNYYLVCLLALARPGNPVYAIREKLDAHN |
| | | GAEPDELMVWRTFRRRGCNRPGLRDSVAKYLTPGNDEPTTEECFA |
| | | AVLAGNPLGATEEGRAILNEGLAQLLGKCTGESGCRNSAKEYLPRFT |
| | | KPDFKGNFEEDTARLNRAEASKRLPFYLHDPATTPNSPALDEFGVLSI |
| | | ALPNPKRAELTGAEARDKLLEFSREWSARLPDGSADWRRLEAKITAL |
| | | GDSLAIPGYTSGSAKGENRFQLYAMFLFCFVEKSDFTLGLLRQTTRR |
| | | PVEGEEPPPRSNHSATAGDPIRIARGGRGYVFRAFTSLKQWGGDTG |
| | | GDLKWPKFDMAAFCEALKALHQVEAKAKQRAEERSKKQALLDYQR |
| | | GRIRRFKPTANSEDATPPPVLAGDPRISRLEQLLNTDLQDEYEMSEG |
| | | VAVAYGLHPRTIRGFREIRKRWISAVGNAPFSEAARATLIACVRSFQS |
| | | ENPGTVGSARLFEALAEESNWIIWREPSAAEQQSWRENADLPESAE |
| | | FALDPLQALTDERELKDEIERLSGPIRFTPADAEHSRRQFYFSDVSQID |
| | | KRNRFRPRLNEVEVELAVRPNGHWTNVWVTLQYSAPRLLRDQLPT |
| | | ADEAGGGWQQAMMAALNLRAPLKKGGEAVSFADCAALSLMPDI |
| | | SPDGEKRLLLNFPVELDGEAIANQLGRSKRWETLQFGGADDESYWL |
| | | RWPKTWIDETKVRRKAAPPKWWLSKEPFSVLGVDLGQRDAAACA |
| | | LVQVSPGNCPTGVCRHVGSADGVDWWATVRSMNMLRLPGENA |
| | | KVMRDGRFQEELSGSRGRSASIDELKEAGDICARLGFVADTILGANG |
| | | RALSFPELNDRLLFSLRSAQSRLARLQSWSCVAHADVPPARREGILR |
| | | DISEAKDDPLGLKPLATAGNLEAIASTLREVILQERTSISAQLVRVADR |
| | | ILPLRGRRWEWVARPESPSNHMLRATAPDTDPRRKLVAGQRGLSL |
| | | ARVEQLESLRQRCQSLNRALMQVPGTPSKLGRRSRGVELPDPCPDL |
| | | LDRLDALKEQRVNQTAHLILAQALGVRLRIHQVSGPQRTRSDSHGE |
| | | YERIPGREPVDFLVLENLDRYLASQGRSRSENSRLMKWCHRAILLKL |
| | | KQLCEPYGLRVLETPAAYSSKFSSRDGTAGFRAVEVTPDDLGSHRW |
| | | RKHSERLADPGASLSRDEREESTRLMAFAERLKALNQDLIARQEAAR |
| | | SSNQPFRPKWRTLLAPQQLGPIFVPAVGKPLQADINAAINIALRAIAS |
| | | PDVDDIHLRIRAARSGDKFVVRAENTRERARWGSAEAEIGLPTGAG |
| | | AKEIEERRSLLTEARVNFFFDPASVAAFDHGKVRDAKLAVTSGRGLW |
| | | GTLRREEWAIVGKINNDRLAAHGLGRPFAERVDL |
| 122 | Cas12 | MSQRDPLPSPTPRAYTLRLSTPDADNGAWRERLWKTHEVVNSGAE |
| | | AFGDWLLSLRGGLDHRLVAVKVRVGRGASKTEREPTEDERRERRIVL |
| | | ALCWLSVESKKGAPQGRVVEDPVDALKRILGQRGLSEADAEQWVF |
| | | DCRDSLSATIRDDATWVDRSAAFDETVQRLGSSLTREHAEQVIIEFF |
| | | GDIHDYLALPKNMEEDGVTSPRGGSGKEFRTQARSWLSENWGAG |
| | | QKGDKAQIVDALRKCSRCIAQEKPSTGSDLLRVLVRSLGGSPEETLRF |
| | | DELRKLVGWRTGRRSTGAFALQRTVDMDRLSESDLESLEAKLTVEID |
| | | AKSPEASRSVPQWVESMRTAVEREVGMPFRSTRDLIGEFGVMLDH |
| | | AARRVSATHSWIKRAEAERRKFEGDAAKLAQIPEEARQWLDQFCE |
| | | DRSQELNALEPYRIRRRAVEGWESVVRTWAKADCKAEGDRVKAVR |
| | | ALQPEIEKFGDAALFEALAAEDALCVWLLDGKATPRPLLDYSAATDA |
| | | QYRKRRFKVPAYRHPDALLHPVFCDFGESRWTIEFSAHRALGRRRK |
| | | AQQLVDNKTVALQKAQERLRKAKAESSRSRAEEKVRAAEHALEAAR |
| | | KKLAFLQYRRALAVCLWTGKGVESTSLRWHSKRFAKDFAIGSSPED |
| | | GASTGVPVARADRLGRASANVPRGASVTISGVFEQKEWNGRLQAP |
| | | RDALNAIAKVRDDETLPAADRSRRVQQMLSRLPWFVTFSARLVPQ |
| | | GPWLDYASEHSALGLRVDPKYWPHAEENRQRKGMARLILSRLPSLR |
| | | VLSVDLGHRYAAACAVWETLSAQEMQRICREMGSAPPSPDDLYLH |
| | | LRRADGNGKQRTTVYRRIGPDTLPDGGQHPAPWARLDRQFLIKLQ |
| | | GEDESARKASDSEIAVVKKFEADLGRPAMQRRSLRVDDLMSAAVRT |
| | | ARLALRRHGDRARIAFNLIADRRFRPGGAEEPLTDETRVDLLADTLA |
| | | TWHGLFSGGQCHDERAEEQWNEHIAPLLASSGASLSVPSDGDAVA |
| | | ATPARRRRKEVRGKLVPAAMELARRDLSQLSVLWVARWHQDDES |
| | | WRARLRWLRDWILPRGAKADSGAIRHVGGLSVTRLATIRSLWQLQ |
| | | KAYRTQPEPEDPRKNVPKKGDTSLDDFGRTILDDLEHLRENRVKQLA |
| | | SRICEAALGVGVEQPNGGAKDPKRPQERRFSPCQAVVIENLTRYRP |
| | | EETRTRRENRQLLNWSAGKVKQYLSEACELHGLHLREVSAAYTSRQ |
| | | DSRTGAPGIRCQDIPVVDFVRENGPCWGRLRSAQQSSGGTAEDQL |
| | | LLALYERWSEKERTWRDQSGNVWAMDGEGRWVARNGTQLPQG |
| | | SGLTPHPIRVPQRGGDIFVSAAPRSPAANGIQADLNAAANIGLWAL |
| | | LDPDWEGRWWRLPCSAMDLKPVKGSVNGSAVIHLDAPLKAAATK |
| | | GAAKKDVVNLWRDISVRSLTEGVWRGYDAYWNLARYRVVQTLRP |
| | | QVGIGSDE |
| 123 | Cas12 | MFKGDAFTGLYEVQKTLRFELVPIGLTQSYLENDWVIQKDKEVEENY |
| | | GKIKAYFDLIHKEFVRQSLENAWLCQLDDFYEKYIELHNSLETRKDKN |
| | | LAKQFEKVMKSLKKEFVSFFDAKWNEWKQKFSFLKKWWIDVLNEK |
| | | EVLDLMAEFYPDEKELFDKFDKFFTYFSNFKESRKNFYADDGRAWAI |
| | | ATRAIDENLITFIKNIEDFKKLNSSFREFVNDNFSEEDKQIFEIDFYNNC |
| | | LLQPWIDKYNKIVWWYSLENWEKVQWLNEKINNFKQNQNKSNSK |
| | | DLKFPRMKLLYKQILGDKEKKVYIDEIRDDKNLIDLIDNSKRRNQIKID |
| | | NANDIINDFINNNAKFELDKIYLTRQSINTISSKYFSSWDYIRWYFWT |
| | | GELQEFVSFYDLKETFWKIEYETLENIFKDCYVKGINTESQNNIVFETQ |
| | | GIYENFLNIFKFEFNQNISQISLLEWELDKIQNEDIKKNEKQVEVIKNY |
| | | FDSVMSVYKMTKYFSLEKWKKRVELDTDNNFYNDFNEYLEGFEIWK |
| | | DYNLVRNYITKKQVNTDKIKLNFDNSQFLTWWDKDKENERLGIILRR |
| | | EWKYYLWILKKWNTLNFGDYLQKEWEIFYEKMNYKQLNNVYRQLP |
| | | RLLFPLTKKLNELKWDELKKYLSKYIQNFWYNEEIAQIKIEFDIFQESKE |
| | | KWEKFDIDKLRKLIEYYKKWVLALYSDLYDLEFIKYKNYDDLSIFYSDV |
| | | EKKMYNLNFTKIDKSLIDGKVKSWELYLFQIYNKDFSESKKEWSTENI |
| | | HTKYFKLLFNEKNLQNLVVKLSWWADIFFRDKTENLKFKKDKNGQEI |
| | | LDHRRFSQDKIMFHISITLNANCWDKYWFNQYVNEYMNKERDIKII |
| | | WIDRWEKHLAYYCVIDKSWKIFNNEIWTLNELNWVNYLEKLEKIESS |
| | | RKDSRISWWEIENIKELKNGYISQVINKLTELIVKYNAIIVFEDLNIWFK |
| | | RWRQKIEKQIYQKLELALAKKLNYLTQKDKKDDEILWNLKALQLVPK |
| | | VNDYQDIWNYKQSWIMFYVRANYTSVTCPNCWLRKNLYISNSATK |
| | | ENQKKSLNSIAIKYNDWKFSFSYEIDDKSWKQKQSLNKKKFIVYSDIE |
| | | RFVYSPLEKLTKVIDVNKKLLELFRDFNLSLDINKQIQEKDLDSVFFKSL |
| | | THLFNLILQLRNSDSKDNKDYISCPSCYYHSNNWLQWFEFNWDAN |
| | | WAYNIARKGIILLDRIRKNQEKPDLYVSDIDWDNFVQSNQFPNTIIPI |
| | | QNIEKQVPLNIKI |
| 124 | Cas12 | MLSFNWVLFFFFSKMSRSIFSPFTNLYPIQKTLRRELKPLNENFQHDP |
| | | ALSALRNSEIPQRDEQREKDYQAIKPLLDEFHNQFITESLHSLEPQDR |
| | | SDFILFYQTYQKKKKNKAEISEKELKSLDEEFESRTKALRNAIGTSFSVT |
| | | AELRKSNPDYVSEKGKPFLTQKSYKILTEAGVLWLLEKKYTSDPEKLAL |
| | | IRRFGNFFTYFTGFNQNRENYYATDEKSTAVAYRAlNENLLTFANNC |
| | | ELFEKLSVLSLSELEKKTFNPDSYSEYLTQSGIVFYNEMLANIRSKANLY |
| | | TQEHKAKLPQPKLLYKQIWSPRGDTIPFDLIASEAEFQETLHTMIRET |
| | | DQRIPEFNKLLEQIFEEKVDLSQIFFSKTSLNIISNRYFSSWHTLLEKGV |
| | | ELKLFKFKKNDEESFKLPAYLSLAELKELLESAPFQMAEKADADEEKH |
| | | HQASLFKLQRENLHLEKSHSNWELLLKSMKSDFESFWTWEGEFWS |
| | | YTLAKKALQSLSALESTNQEHKNLIKMLLDNALYAYRMLKWFKVDTS |
| | | KLGFVPEGEFYPSLDQLLQDYPLPKWYDMIRNYLTRKTYSQAKLKLN |
| | | FDCSTLLNGRDKNKEIQNLSVILRKDGKFYLAIMKKDQNKFFENSALY |
| | | EGNLGTMEKMDYKLLPWANKMLPKCLMPGSDKKKYGASDQVLEL |
| | | YAKGSFKKSEKSFNLADLHTLIDFYKLALPKYEDWKVFNFQFQATEN |
| | | YQDISQFYREVEQQGYLLNWRKVNEKLIKQGIKDWSLFLFQISSKDF |
| | | EGKSKTPDLQTLYWQQLFEFSTNVKLNGEAEIFFRPWSMKKEKKKL |
| | | KVDNYDVFKHKRYTEDKILFHVPITLWFGNNEVSPSAPSKFNQKLN |
| | | QELIIPHFDDLHVIGVDRWEKHLAFYSVVSVKTGKIVKQGTLNLLNG |
| | | TDYEAKLSQKAENRLYARQNRDTIEKIADLKNGYISQVVNKLVELVLE |
| | | YNAVIVFEDLNAGFKRGRQKIEQSVYQKLELALAKKLNFIVKKEKAVG |
| | | EPWSVTSAYQLAPQINTFWDIKWKQRGIMLYTRANYTSVTDPLTG |
| | | WRKQYYFKKGSSEEMKAQFFKSFKNLTRDAGQEAYIFDDGTWLLYS |
| | | NVERRRGKRGDHRERTQIKYDPSLELDTLFSKYQIEKSDSLFDQLKNR |
| | | ELPQTFWTSFFRIIDLIMQIRNTDDEGRDIILSPIGNPQERFDSRKRYN |
| | | QLPRDEKGGIIEESAFEYPTSWDANGAYNIARKGVMMLERIKENPE |
| | | KPDLLIRDAEWDKKITRK |
| 125 | Cas12 | MLDKFASLYPVTKTLRFRLLPQGRTEENMQVAKVLENDLERSEAAA |
| | | WKGLIKKYHLQFISDTLSGSTLSWQALTETLDKFKADHTATAELDSA |
| | | LAAYRCKLAELFTKSPKYKVMATPVSIIKEILKTETDPENIAALNKLNG |
| | | YTYIIFDYVSTRMLTYSADAKATSLAYRLVDENYLRFYQD1SAAAEISA |
| | | VLEEAGFDNAEVEAFIRTDYNTCLTSEGIASFNAAAGSINQFVNVLL |
| | | QQNPVLQSEPALRRHLQPLYKMLLDEAESKIIKFEDYGQLRDAVENF |
| | | RRNFQDLPQSLIDIFAGRYDYSKIYVGYKYLNEASSQIAGGYNWKLLE |
| | | NALEDFYSKPYLVNGKLPVKYKTVVNKKMNQLAYSFTELQEALDAG |
| | | DSGSSITDLFGKYAELHAAYAAADGNVFYKEYDRKSIASLKNYLDAV |
| | | NAIARFIKIFAAPEVYVKDEGFYGIVDGAADKLRDFDLLYNMVRNYIT |
| | | KKPYKKSKVALTFNSSSFGRGWDENKIYDELTTIFTYNGKYYLGVINK |
| | | NDKPDLAAAVSKDEGGYKRMVYKTFDIVKQLPRLSFTKAVKAHFAE |
| | | SDEDFIFDGPKFAKPLRVPKEIYLQSFTDNGDKLADSAKKYTKAYLD |
| | | MSGDYKGYYEAIIKRIDYTKEFLSAYKSTSIYDLAFLKPAGKAAGSLCW |
| | | TRHI |
| 126 | Cas12 | MNRIYQGRVTKVEILNGKNADGQPQELPNWQTALWQHHELFQD |
| | | AVNYYLFCLAALASSASSPMGKLRNQLGSVWEPFGRTGRRFKGLRD |
| | | SVGPYLLPDRPTPSIEEAFAAALAGNNSQPELLQLAVDALVEDLGGD |
| | | AAIQQEGRGYLPRLCSPAYNGQFPRGANSLQKEAAKIRLPTLLHNVA |
| | | TSRNLDALAAELEFGFFANPVPNAGPIAGQEARNKLLEALAWLKAK |
| | | DTRLTHSANELEKRIGVLPDSVSFPTYSGGSINKEALKHRFFAYLLFQ |
| | | HVERSLVTFEILRDSYPTPKPSARKSSKATPDTMAQLTQFGDDPVKL |
| | | ARGTRGYVFRAFTSLPCWGAKAPSDILWSEFDIAAFKEALKTANQFR |
| | | LKTKERLDKADELAGELAWMNGEKSKFKPSEHSEQEPPAVLKGDPR |
| | | FEVLKQLFEVELAEEHYLAEGESVIYGLHPRTLRCYRELVERWNKTVQ |
| | | PGEVFTEATSKKLVASTDKFQAENKERIGSVTLFKKLLERDYWCLWQ |
| | | TPDVQTVAARQKAGFSSNIIEDYQRYLELQTDIARLKEPIRFTPADAE |
| | | QSRRLFMFSDLAGKSKHKHLPNATPYGFAVDVALAASEGGVWKET |
| | | RVRLHYSAPRLRRDGLRKGVGEDLKRTAWLQPMVAALNLSEPAPQ |
| | | DFSKCAVSLMPDKSWKGLRHLLNFPVTLDPTVVQKAIGSQARWAN |
| | | QFVAFGKGATEQKFFLRWPEEVSASQKKAGPWWDALRAFSCLSVD |
| | | LGQRDAGAFALLDVRANADWGKKPSRFIGEADGRNWRAALAAAG |
| | | LLRLPGEDALVWRDGKWQEELYGEKGRLATKEEWLETNAIFSALEQ |
| | | NSEEWIGADPKRRSFPELNSKLLVVARRGQSWLARLHRWLWMLG |
| | | DENKRERALRELLEQERQEAWRKRAEGKDLLALKQSLTAEIQRLNGL |
| | | LPEQLVFLANRCLPLRNRKWAWNQHPDKAFAEKGCHLLEMVEAPS |
| | | HLPLLAGQRGISFERIGQLEELRRRFQSLNRVLQRELGAPPKSGRELR |
| | | DDLVPDCCPDILAKLDRVKEQRVNQTAHLIVAQVLGVQLRKHQTPE |
| | | SERTERDLHGEYEIMPGRKPVDFIVLEDLSKYLSSQARGRGENVRLM |
| | | KWCHRQVTAKVKELCEPFGLPVLETPAAYSSKFCSRTGVAGFRGVE |
| | | VTLKDRQSFPWSKRLEEDSAEVAELFGWLETASAGHKTKQPRCLLA |
| | | PMSLGPLFIPVTSQAPIMQADINAAINLGLRAMAAPSVHEIHVRIRS |
| | | EAKDGQFRVRAQSKREKARWGNEPPPIKMLVETQRVALAREGGH |
| | | PNFFVDPLKAATFDRAEVEGLGLPVASGRGLWKSVRDAEWKRCREI |
| | | NLERMRRWGFIMEAPRTLQVSRKEDEDDLTF |
| 127 | Cas12 | MAVRSVKLKLLVPRDGSAESVRKRKALWATHQFVNDAAAAYAELLL |
| | | EMRQEDVCRGTDDHGKDVIEPAAHWQAKLRARLAAKQLPPVAVA |
| | | EALPLLKAFYGSRLIKSFVANDKGVAGTGNATDLNTWLSGLVDPASV |
| | | AGEKTELRKQLLAELPLCEAADADFEGAARKMLAKSDAREALLEGP |
| | | GTGVGWPAAYNANPTDSVWLDMLHKAAAKARLELADTTVSELKK |
| | | LGVFPLLQAASSNRVFGSGVLNPFERMAAAQAAAALLPWETKRHE |
| | | MRKRRDKFADQLNQWDTEFGASHATALAAIRAFEAEESERARRESL |
| | | GNEGTGYRIGGRELRDAWTLLRDWLKGHSTATAAAREDKVRELQA |
| | | KQGRSFGSHRLLSWLAKPAQQWLADHSAGDVVTRIAVRNARQRK |
| | | LDTARTLPIWTGADAVKHPRFANFDPPNNTNQPGFDLRAGTQKGR |
| | | LTLRLSLLTERADGLLLAQDHDFQLVPSRQMAEIVLHKDGKERALSW |
| | | QSQDGIGRQVGDVGGSALLFSRDHAECLLERKQITRLERGAWPAAL |
| | | PVWFKLSLDIGAEHKALLKQRFKWGVWLNSALVTRNAKDAKGVPP |
| | | PVGTRVLAVDLGLRSAATVSVWQVVDAATPVVAGKWRVPLSDTLS |
| | | AVHERSAMLALPGEHVDAGVLAARRAANEKLAGLLAATSHLSTVFK |
| | | LGRAEQGDRRRELLERLGEGDDRRARAAVATTAAERDGLRAVLGA |
| | | TQDAWAGAVAAVWRRLETDLAGAIAAYRKQQREDVQLRREARHG |
| | | PGASQLPKQAAAERLLGGKSAWQIEYKERVRKLLTRWIMRQRPGD |
| | | TAVRRLARKDLGKYCGGLLDHLTALKEDRAKTTADLIVQAARGRVR |
| | | AHKDAHGRQQDRELWLAKYAPCDLIVMEDLGRYRFATDRPPSENR |
| | | QLMQWTHREVFRLVQMQAEVEGIQVLETGAEFSSKFDARTWAPG |
| | | VRCEPITKLWVERYRNGEMPWLADKADEWRREGIELAQLVPGQLL |
| | | PTGSGEQFVAVSATGGLRVRHADLNAAQCIALRALTGHGTAFRLTA |
| | | RRLGDVFVSAKGLGKRPQGALWREFGSALPPAVVVLRPAGEVRYAL |
| | | RPFASARDAAAALGLQLGALRNVDATDAESDAEDGDLAELLAGAD |
| | | PDRATFFRDPSGDVHGGAWVQAKVFWAEVRRHVRLGLQAQGLLP |
| | | AAARSSEPRQMQLPLAGALPGDDIPL |
| 128 | Cas12 | MSTQKNPFNQFTNLYELQKTLRFELRPVPETKKLLEKGEGKNLIQMD |
| | | LEIDRLYEKEMKPMFNILHEKFINESLGLVKLDCKKLKKLENLLAEADK |
| | | LRKQIKEGRKNKNNISEVEKRLKIIIGDNSQGKNKNGEIAVLQDELRV |
| | | LIVKAFNLTADKWKKELNNKETLLPEKKGKRKIKIKKSGPKILQEENVL |
| | | AILAYFNPDKADIIKKFAGFFTYFSGFNQNRANYYTVKALATGVANR |
| | | AINRNFLIFLANRKDFARFKERLPRLAEFDNYFELENYEKYLSQ.TGIEE |
| | | YNDQIGKIKQIVNLEHNQQQKDNKFQLKGLATLEKQIGCRTKKQRE |
| | | EGGDKSAPKFLEKVGLGFQVSQDDDGEYLIWECLNYINKELAGKLKS |
| | | IKDNYQKFFADWRTGAYDLEKIWFRKEALNTISGRWFGGNNWFIIG |
| | | KALALTGVGKFDKRENTYKIPEFVSLAEIKTAFEMLENGVNYDFKKSK |
| | | KKKEGDDTDVVKYSADNLFKEEYKKKGLIKNSLFETMLAVWQSEIKR |
| | | KFEQIFDGYKLEKDDVFGRKKGEWVEPFIENFQKVSQEKFDRGVKD |
| | | ENGRSIHTEVVKNLIEEGYLRLFQLTKYHNLDKKGERDPRPFDGNFY |
| | | ATLDEFWKDNIVVVYHKALQSTLTKKPYSEDKIKLNFENGSLLGGFS |
| | | DGQERSKAGVVLKNKNKFYLGILIDRGFFRTDKANPVYDNAQNNE |
| | | WERLILTNLKFQTLAGKGFLGKHGVSYGEMGKDNPMMAVEYLQKF |
| | | IKLKYLDKYPALNEVAHKKYTIKKEFDADVKNALKDCFTMNFKPVDF |
| | | GMIRQGLTESLFYLFEIVNKDISSQAKNGKNVHTLYWEALFGDQNLK |
| | | KPILALNGGAEIFYRESQREKLEKKLDKSGKEVLDHKRYGQDKYFLHA |
| | | SITINYGQPKNIKFKEVINEKISQNADRVNIIGIDRGEKHLLYYSVVSPE |
| | | GVLLEQGSFNQIETKNKVDIKAVKAEYGERGELKKVELVPTGKKVKY |
| | | VDYQILLDYYEKKRNLARRDWQTIGKIKDLKDGYLSQTVHRIYQLILK |
| | | YNAVVAMEDLNVEFKAKRAAKVEKSVYKNFEMALAKKLNHLILKDR |
| | | RADEIGGALRAYQLTPAIPANDVGKFDKAKQWGIMFYVRANYTSTT |
| | | DPLTGWRKHKYISNSEKIDNIQKFFSPGDGIQIDYDTEKQCFKFSYDH |
| | | ELEGGAKKHWELFACDGLERFYWDNRERQIKKYNLYEEFEKLLGGL |
| | | RKEENINIQIDGVSEFRWKDLVFFWNLLNQIRNTDRSAQGDENDFL |
| | | QSPVWSEKYNCFYDSRKAPNNMPNNGDANGAFNIARKGQLILERI |
| | | KKCSDIPKFGNDNNGKNPENNYFISDADWDKFAQK |
| 129 | Cas12 | MAVKSIKVKLRLDDMPEIRAGLWKLHKEVNAGVRYYTEWLSLLRQE |
| | | NLYRRSPNGDGEQECDKTAEECKAELLERLRARQVENGHRGPAGS |
| | | DDELLQLARQLYELLVPQAIGAKGDAQQIARKFLSPLADKDAVGGL |
| | | GIAKAGNKPRWVRMREAGEPGWEEEKEKAETRKSADRTADVLRAL |
| | | ADFGLKPLMRVYTDSEMSSVEWKPLRKGQAVRTWDRDMFQQAIE |
| | | RMMSWESWNQRVGQEYAKLVEQKNRFEQKNFVGQEHLVHLVN |
| | | QLQQDMKEASPGLESKEQTAHYVTGRALRGSDKVFEKWGKLAPDA |
| | | PFDLYDAEIKNVQRRNTRRFGSHDLFAKLAEPEYQALWREDASFLTR |
| | | YAVYNSILRKLNHAKMFATFTLPDATAHPIWTRFDKLGGNLHQYTFL |
| | | FNEFGERRHAIRFHKLLKVENGVAREVDDVTVPISMSEQLDNLLPRD |
| | | PNEPIALYFRDYGAEQHFTGEFGGAKIQCRRDQLAHMHRRRGARD |
| | | VYLNVSVRVQSQSEARGERRPPYAAVFRLVGDNHRAFVHFDKLSDY |
| | | LAEHPDDGKLGSEGLLSGLRVMSVDLGLRTSASISVFRVARKDELKP |
| | | NSKGRVPFFFPIKGNDNLVAVHERSQLLKLPGETESKDLRAIREERQR |
| | | TLRQLRTQLAYLRLLVRCGSEDVGRRERSWAKLIEQPVDAANHMTP |
| | | DWREAFENELQKLKSLHGICSDKEWMDAVYESVRRVWRHMGKQ |
| | | VRDWRKDVRSGERPKIRGYAKDVVGGNSIEQIEYLERQYKFLKSWS |
| | | FFGKVSGQVIRAEKGSRFAITLREHIDHAKEDRLKKLADRIIMEALGY |
| | | VYALDERGKGKWVAKYPPCQLILLEELSEYQFNNDRPPSENNQLMQ |
| | | WSHRGVFQELINQAQVHDLLVGTMYAAFSSRFDARTGAPGIRCRR |
| | | VPARCTQEHNPEPFPWWLNKFVVEHTLDACPLRADDLIPTGEGEIF |
| | | VSPFSAEEGDFHQIHADLNAAQNLQQRLWSDFDISQIRLRCDWGE |
| | | VDGELVLIPRLTGKRTADSYSNKVFYTNTGVTYYERERGKKRRKVFA |
| | | QEKLSEEEAELLVEADEAREKSVVLMRDPSGIINRGNWTRQKEFWS |
| | | MVNQRIEGYLVKQIRSRVPLQDSACENTGDI |
| 130 | Cas12 | MSTQKNPFNQFTNLYELQKTLRFELRPVPETKKLLEKGEGKNLIQMD |
| | | LEIDRLYEKEMKPMFNILHEKFINESLGLVKLDCKKLKKLENLLAEADK |
| | | LRKQIKEGRKNKNNISEVEKRLKIIIGDNSQGKNKNGEIAVLQDELRV |
| | | LIVKAFNLTADKWKKELNNKETLLPEKKGKRKIKIKKSGPKILQEENVL |
| | | AILAYFNPDKADIIKKFAGFFTYFSGFNQNRANYYTVKALATGVANR |
| | | AINRNFLIFLANRKDFARFKERLPRLAEFDNYFELENYEKYLSQTGIEE |
| | | YNDQIGKIKQIVNLEHNQQQKDNKFQLKGLATLEKQIGCRTKKQRE |
| | | EGGDKSAPKFLEKVGLGFQVSQDDDGEYLIWECLNYINKELAGKLKS |
| | | IKDNYQKFFADWRTGAYDLEKIWFRKEALNTISGRWFGGNNWFIIG |
| | | KALALTGVGKFDKRENTYKIPEFVSLAEIKTAFEMLENGVNYDFKKSK |
| | | KKKEGDDTDVVKYSADNLFKEEYKKKGLIKNSLFETMLAVWQSEIKR |
| | | KFEQIFDGYKLEKDDVFGRKKGEWVEPFIENFQKVSQEKFDRGVKD |
| | | ENGRSIHTEVVKNLIEEGYLRLFQLTKYHNLDKKGERDPRPFDGNFY |
| | | ATLDEFWKDNIVVVYHKALQSTLTKKPYSEDKIKLNFENGSLLGGFS |
| | | DGQERSKAGVVLKNKNKFYLGILIDRGFFRTDKANPVYDNAQNNE |
| | | WERLILTNLKFQTLAGKGFLGKHGVSYGEMGKDNPMMAVEYLQKF |
| | | IKLKYLDKYPALNEVAHKKYTIKKEFDADVKNALKDCFTMNFKPVDF |
| | | GMIRQGLTESLFYLFEIVNKDISSQAKNGKNVHTLYWEALFGDQNLK |
| | | KPILALNGGAEIFYRESQREKLEKKLDKSGKEVLDHKRYGQDKYFLHA |
| | | SITINYGQPKNIKFKEVINEKISQNADRVNIIGIDRGEKHLLYYSVVSPE |
| | | GVLLEQGSFNQIETKNKVDIKAVKAEYGERGELKKVELVPTGKKVKY |
| | | VDYQILLDYYEKKRNLARRDWQTIGKIKDLKDGYLSQTVHRIYQLILK |
| | | YNAVVAMEDLNVEFKAKRAAKVEKSVYKNFEMALAKKLNHLILKDR |
| | | RADEIGGALRAYQLTPAIPANDVGKFDKAKQWGIMFYVRANYTSTT |
| | | DPLTGWRKHKYISNSEKIDNIQKFFSPGDGIQIDYDTEKQCFKFSYDH |
| | | ELEGGAKKHWELFACDGLERFYWDNRERQIKKYNLYEEFEKLLGGL |
| | | RKEENINIQIDGVSEFRWKDLVFFWNLLNQIRNTDRSAQGDENDFL |
| | | QSPVWSEKYNCFYDSRKAPNNMPNNGDANGAFNIARKGQLILERI |
| | | KKCSDIPKFGNDNNGKNPENNYFISDADWDKFAQK |
| 131 | Cas12 | MAIRSIKLKLKTHTGPEAQNLRKGIWRTHRLLNEGVAYYMKMLLLF |
| | | RQESTGERPKEELQEELICHIREQQQRNQADKNTQALPLDKALEALR |
| | | QLYELLVPSSVGQSGDAQIISRKFLSPLVDPNSEGGKGTSKAGAKPT |
| | | WQKKKEANDPTWEQDYEKWKKRREEDPTASVITTLEEYGIRPIFPLY |
| | | TNTVTDIAWLPLQSNQFVRTWDRDMLQQAIERLLSWESWNKRVQ |
| | | EEYAKLKEKMAQLNEQLEGGQEWISLLEQYEENRERELRENMTAA |
| | | NDKYRITKRQMKGWNELYELWSTFPASASHEQYKEALKRVQQRLR |
| | | GRFGDAHFFQYLMEEKNRLIWKGNPQRIHYFVARNELTKRLEEAKQ |
| | | SATMTLPNARKHPLWVRFDARGGNLQDYYLTAEADKPRSRRFVTF |
| | | SQLIWPSESGWMEKKDVEVELALSRQFYQQVKLLKNDKGKQKIEFK |
| | | DKGSGSTFNGHLGGAKLQLERGDLEKEEKNFEDGEIGSVYLNVVIDF |
| | | EPLQEVKNGRVQAPYGQVLQLIRRPNEFPKVTTYKSEQLVEWIKASP |
| | | QHSAGVESLASGFRVMSIDLGLRAAAATSIFSVEESSDKNAADFSYW |
| | | IEGTPLVAVHQRSYMLRLPGEQVEKQVMEKRDERFQLHQRVKFQI |
| | | RVLAQIMRMANKQYGDRWDELDSLKQAVEQKKSPLDQTDRTFWE |
| | | GIVCDLTKVLPRNEADWEQAVVQIHRKAEEYVGKAVQAWRKRFAA |
| | | DERKGIAGLSMWNIEELEGLRKLLISWSRRTRNPQEVNRFERGHTS |
| | | HQRLLTHIQNVKEDRLKQLSHAIVMTALGYVYDERKQEWCAEYPAC |
| | | QVILFENLSQYRSNLDRSTKENSTLMKWAHRSIPKYVHMQAEPYGI |
| | | QIGDVRAEYSSRFYAKTGTPGIRCKKVRGQDLQGRRFENLQKRLVN |
| | | EQFLTEEQVKQLRPGDIVPDDSGELFMTLTDGSGSKEVVFLQADINA |
| | | AHNLQKRFWQRYNELFKVSCRVIVRDEEEYLVPKTKSVQAKLGKGLF |
| | | VKKSDTAWKDVYVWDSQAKLKGKTTFTEESESPEQLEDFQEIIEEAE |
| | | EAKGTYRTLFRDPSGVFFPESVWYPQKDFWGEVKRKLYGKLRERFL |
| | | TKAR |
| 132 | Cas12 | MRKKLFKGYILHNKRLVYTGKAAIRSIKYPLVAPNKTALNNLSEKIIYD |
| | | YEHLFGPLNVASYARNSNRYSLVDFWIDSLRAGVIWQSKSTSLIDLIS |
| | | KLEGSKSPSEKIFEQIDFELKNKLDKEQFKDIILLNTGIRSSSNVRSLRG |
| | | RFLKCFKEEFRDTEEVIACVDKWSKDLIVEGKSILVSKQFLYWEEEFGI |
| | | KIFPHFKDNHDLPKLTFFVEPSLEFSPHLPLANCLERLKKFDISRESLLG |
| | | LDNNFSAFSNYFNELFNLLSRGEIKKIVTAVLAVSKSWENEPELEKRL |
| | | HFLSEKAKLLGYPKLTSSWADYRMIIGGKIKSWHSNYTEQLIKVREDL |
| | | KKHQIALDKLQEDLKKVVDSSLREQIEAQREALLPLLDTMLKEKDFSD |
| | | DLELYRFILSDFKSLLNGSYQRYIQTEEERKEDRDVTKKYKDLYSNLRN |
| | | IPRFFGESKKEQFNKFINKSLPTIDVGLKILEDIRNALETVSVRKPPSITE |
| | | EYVTKQLEKLSRKYKINAFNSNRFKQITEQVLRKYNNGELPKISEVFYR |
| | | YPRESHVAIRILPVKISNPRKDISYLLDKYQISPDWKNSNPGEVVDLIEI |
| | | YKLTLGWLLSCNKDFSMDFSSYDLKLFPEAASLIKNFGSCLSGYYLSK |
| | | MIFNCITSEIKGMITLYTRDKFVVRYVTQMIGSNQKFPLLCLVGEKQT |
| | | KNFSRNWGVLIEEKGDLGEEKNQEKCLIFKDKTDFAKAKEVEIFKNNI |
| | | WRIRTSKYQIQFLNRLFKKTKEWDLMNLVLSEPSLVLEEEWGVSWD |
| | | KDKLLPLLKKEKSCEERLYYSLPLNLVPATDYKEQSAEIEQRNTYLGLD |
| | | VGEFGVAYAVVRIVRDRIELLSWGFLKDPALRKIRERVQDMKKKQV |
| | | MAVFSSSSTAVARVREMAIHSLRNQIHSIALAYKAKIIYEISISNFETG |
| | | GNRMAKIYRSIKVSDVYRESGADTLVSEMIWGKKNKQMGNHISSY |
| | | ATSYTCCNCARTPFELVIDNDKEYEKGGDEFIFNVGDEKKVRGFLQK |
| | | SLLGKTIKGKEVLKSIKEYARPPIREVLLEGEDVEQLLKRRGNSYIYRCP |
| | | FCGYKTDADIQAALNIACRGYISDNAKDAVKEGERKLDYILEVRKLW |
| | | EKNGAVLRSAKFL |
| 133 | Cas12 | MSTQKNPFNQFTNLYELQKTLRFELRPVPETKKLLEKGEGKNLIQMD |
| | | LEIDRLYEKEMKPMFNILHEKFINESLGLVKLDCKKLKKLENLLAEADK |
| | | LRKQIKEGRKNKNNISEVEKRLKIIIGDNSQGKNKNGEIAVLQDELRV |
| | | LIVKAFNLTADKWKKELNNKETLLPEKKGKRKIKIKKSGPKILQEENVL |
| | | AILAYFNPDKADIIKKFAGFFTYFSGFNQNRANYYTVKALATGVANR |
| | | AINRNFLIFLANRKDFARFKERLPRLAEFDNYFELENYEKYLSQ.TGIEE |
| | | YNDQIGKIKQIVNLEHNQQQKDNKFQLKGLATLEKQIGCRTKKQRE |
| | | EGGDKSAPKFLEKVGLGFQVSQDDDGEYLIWECLNYINKELAGKLKS |
| | | IKDNYQKFFADWRTGAYDLEKIWFRKEALNTISGRWFGGNNWFIIG |
| | | KALALTGVGKFDKRENTYKIPEFVSLAEIKTAFEMLENGVNYDFKKSK |
| | | KKKEGDDTDVVKYSADNLFKEEYKKKGLIKNSLFETMLAVWQSEIKR |
| | | KFEQIFDGYKLEKDDVFGRKKGEWVEPFIENFQKVSQEKFDRGVKD |
| | | ENGRSIHTEVVKNLIEEGYLRLFQLTKYHNLDKKGERDPRPFDGNFY |
| | | ATLDEFWKDNIVVVYHKALQSTLTKKPYSEDKIKLNFENGSLLGGFS |
| | | DGQERSKAGVVLKNKNKFYLGILIDRGFFRTDKANPVYDNAQNNE |
| | | WERLILTNLKFQTLAGKGFLGKHGVSYGEMGKDNPMMAVEYLQKF |
| | | IKLKYLDKYPALNEVAHKKYTIKKEFDADVKNALKDCFTMNFKPVDF |
| | | GMIRQGLTESLFYLFEIVNKDISSQAKNGKNVHTLYWEALFGDQNLK |
| | | KPILALNGGAEIFYRESQREKLEKKLDKSGKEVLDHKRYGQDKYFLHA |
| | | SITINYGQPKNIKFKEVINEKISQNADRVNIIGIDRGEKHLLYYSVVSPE |
| | | GVLLEQGSFNQIETKNKVDIKAVKAEYGERGELKKVELVPTGKKVKY |
| | | VDYQILLDYYEKKRNLARRDWQTIGKIKDLKDGYLSQTVHRIYQLILK |
| | | YNAVVAMEDLNVEFKAKRAAKVEKSVYKNFEMALAKKLNHLILKDR |
| | | RADEIGGALRAYQLTPAIPANDVGKFDKAKQWGIMFYVRANYTSTT |
| | | DPLTGWRKHKYISNSEKIDNIQKFFSPGDGIQIDYDTEKQCFKFSYDH |
| | | ELEGGAKKHWELFACDGLERFYWDNRERQIKKYNLYEEFEKLLGGL |
| | | RKEENINIQIDGVSEFRWKDLVFFWNLLNQIRNTDRSAQGDENDFL |
| | | QSPVWSEKYNCFYDSRKAPNNMPNNGDANGAFNIARKGQLILERI |
| | | KKCSDIPKFGNDNNGKNPENNYFISDADWDKFAQK |
| 134 | Cas12 | MAQASSTPAVSPRPRPRYREERTLVRKLLPRPGQSKQEFRENVKKLR |
| | | KAFLQFNADVSGVCQWAIQFRPRYGKPAEPTETFWKFFLEPETSLP |
| | | PNDSRSPEFRRLQAFEAAAGINGAAALDDPAFTNELRDSILAVASRP |
| | | KTKEAQRLFSRLKDYQPAHRMILAKVAAEWIESRYRRAHQNWERN |
| | | YEEWKKEKQEWEQNHPELTPEIREAFNQIFQQLEVKEKRVRICPAA |
| | | RLLQNKDNCQYAGKNKHSVLCNQFNEFKKNHLQGKAIKFFYKDAEK |
| | | YLRCGLQSLKPNVQGPFREDWNKYLRYMNLKEETLRGKNGGRLPH |
| | | CKNLGQECEFNPHTALCKQYQQQLSSRPDLVQHDELYRKWRREYW |
| | | REPRKPVFRYPSVKRHSIAKIFGENYFQADFKNSVVGLRLDSMPAGQ |
| | | YLEFAFAPWPRNYRPQPGETEISSVHLHFVGTRPRIGFRFRVPHKRS |
| | | RFDCTQEELDELRSRTFPRKAQDQKFLEAARKRLLETFPGNAEQELR |
| | | LLAVDLGTDSARAAFFIGKTFQQAFPLKIVKIEKLYEQWPNQKQAGD |
| | | RRDASSKQPRPGLSRDHVGRHLQKMRAQASEIAQKRQELTGTPAP |
| | | ElIIDQAAKKATLQPFDLRGLTVHTARMIRDWARLNARQIIQLAEE |
| | | NQVDLIVLESLRGFRPPGYENLDQEKKRRVAFFAHGRIRRKVTEKAV |
| | | ERGMRVVTVPYLASSKVCAECRKKQKDNKQWEKNKKRGLFKCEGC |
| | | GSQAQVDENAARVLGRVFWGEIELPTAIP |
| 135 | Cas12 | MAVRSVKLKLLVPRDGSAESVRKRKALWATHQFVNDAAAAYAELLL |
| | | EMRQEDVCRGTDDHGKDVIEPAAHWQAKLRARLAAKQLPPVAVA |
| | | EALPLLKAFYGSRLIKSFVANDKGVAGTGNATDLNTWLSGLVDPASV |
| | | AGEKTELRKQLLAELPLCEAADADFEGAARKMLAKSDAREALLEGP |
| | | GTGVGWPAAYNANPTDSVWLDMLHKAAAKARLELADTTVSELKK |
| | | LGVFPLLQAASSNRVFGSGVLNPFERMAAAQAAAALLPWETKRHE |
| | | MRKRRDKFADQLNQWDTEFGASHATALAAIRAFEAEESERARRESL |
| | | GNEGTGYRIGGRELRDAWTLLRDWLKGHSTATAAAREDKVRELQA |
| | | KQGRSFGSHRLLSWLAKPAQQWLADHSAGDVVTRIAVRNARQRK |
| | | LDTARTLPIWTGADAVKHPRFANFDPPNNTNQPGFDLRAGTQKGR |
| | | LTLRLSLLTERADGLLLAQDHDFQLVPSRQMAEIVLHKDGKERALSW |
| | | QSQDGIGRQVGDVGGSALLFSRDHAECLLERKQITRLERGAWPAAL |
| | | PVWFKLSLDIGAEHKALLKQRFKWGVWLNSALVTRNAKDAKGVPP |
| | | PVGTRVLAVDLGLRSAATVSVWQVVDAATPVVAGKWRVPLSDTLS |
| | | AVHERSAMLALPGEHVDAGVLAARRAANEKLAGLLAATSHLSTVFK |
| | | LGRAEQGDRRRELLERLGEGDDRRARAAVATTAAERDGLRAVLGA |
| | | TQDAWAGAVAAVWRRLETDLAGAIAAYRKQQREDVQLRREARHG |
| | | PGASQLPKQAAAERLLGGKSAWQIEYKERVRKLLTRWIMRQRPGD |
| | | TAVRRLARKDLGKYCGGLLDHLTALKEDRAKTTADLIVQAARGRVR |
| | | AHKDAHGRQQDRELWLAKYAPCDLIVMEDLGRYRFATDRPPSENR |
| | | QLMQWTHREVFRLVQMQAEVEGIQVLETGAEFSSKFDARTWAPG |
| | | VRCEPITKLWVERYRNGEMPWLADKADEWRREGIELAQLVPGQLL |
| | | PTGSGEQFVAVSATGGLRVRHADLNAAQCIALRALTGHGTAFRLTA |
| | | RRLGDVFVSAKGLGKRPQGALWREFGSALPPAVVVLRPAGEVRYAL |
| | | RPFASARDAAAALGLQLGALRNVDATDAESDAEDGDLAELLAGAD |
| | | PDRATFFRDPSGDVHGGAWVQAKVFWAEVRRHVRLGLQAQGLLP |
| | | AAARSSEPRQMQLPLAGALPGDDIPL |
| 136 | Cas12 | MNRIYQGRVSKIEIKDSEGNFRNVPVGSPDTCPLWRHHRIFQDAVN |
| | | YYLVALGALAGTGSENAFVGLGSKDRVIHDLYSRLFDSWERFPRDM |
| | | HGASSLRDSLRRTLPGLSERASLQDAFDAILSGNEANARERVLSLLSLI |
| | | QDLGGDIQKGSKRYFPFFCEPATKATFPRARVGLLKVEGKDFVPRLL |
| | | WSSDLEIAPDQVVEQLKFEYFANPNESVQPIEGNEARVRLIEALDNP |
| | | QLGIELPIEILSDLRKRVHLIETDIRIPRYFFGGAGAELRKFRLDLFLIAA |
| | | YVTPDPSILRALRNSFKEPSASKSSKKKDETEEVENLLRSLGDDPLILA |
| | | RGERGFVFPSFTSLPTWVGANAQKPIWRDFDIAAFAEALKSLNQFT |
| | | AKTEEREEKLKKAEETLHYMLGISDAIPRSSDSETEEQAPSRPGKDPR |
| | | WPLVAQLEKELGENLSEGTWQLSRSAMRGLRDIIGLWRKHPGASV |
| | | VTLQKDVKTYQADEKHKREIGSVQLFLLLCEERYHALWQTETDDER |
| | | GDESEENDDPARILSDAIEVHQIRREVERFREPIRLTPAEPVFSRRLF |
| | | MFSDLTDKLAKVKFGETTEENSEVKSQFVEAAIALKEGENLKEARVRI |
| | | TFSAPRLHRDELLGGAESRWLQPITAALGFSNPAPSVKFDSAVALM |
| | | PDHMDDGRIRHLLNFPVNFDSAWLHQSIGKADLWKSQFNGTKDK |
| | | NLHLHWAGTARDTTRKNTWWENRTIIENGFTVLSNDLGQRSAGA |
| | | WALLKVTCSRPDTKHPVRSIGHDGTREWFATVLATGIHRLPGEDQR |
| | | ILKNGKWATEQSGKKGRNATFSEYEAACVLAKNLGCESVENWLGM |
| | | SGEKSYPALNDQLVKIANRRITRLGTYHRWSCFSPEKFEDPARRANV |
| | | IGGQLAELSAYQDENVTVSADILKSGDFEGFRHRAGAAFEALRTELE |
| | | VHLVNLANLTAPLRQKVWSWQKRPDSSGYGDLLMVDLDDCHPKIR |
| | | GQRGLSMARLEQLEGLRRLFLRYNRSLDRSPGIPAKFGREDVGRTSG |
| | | EPCQALLVKIDRMKEQRVNQTAHLILAQALGVRLCPHRIEENERKSR |
| | | DLHGEYEKIPGREPVDFIVIEDLSRYLSSQGRAPSENSRLMKWAHRA |
| | | VRDKLKMLAEEPFGIPVVETVPAYSSRFHALNGQAGSRLHELHELEA |
| | | YQQQSLINLAAKTDFQNRDRSKAAGELFEQFQALAKLNERRRAEGK |
| | | KVPRTLYYPKSGGPLFLASRDGDTIHADVNAAINLGLRAIAAPACIDI |
| | | HRRLRATKEKEVYRPRVGNAREKSAFSKDDIIQPSGAPSKKFASSSSP |
| | | NFFYEPEDLKQANGEPLFDRAMFGEYSLVSGVSLWSMVNNAIYIRC |
| | | VELNRTRLHGKDPDDQIPM |
| 137 | Cas12 | MADDLSTQRAYTLRLQGTDPEDQSWRDALWMTHEAVNAGGRAF |
| | | GDWLLTLRGGIAHELADTPVKGKKDITDELRKKRRILLALSWLSVESR |
| | | RGAPDKFIVAGGEEPAGSRNEKVLQALKEILKRRGLSAEESESWMS |
| | | DCRASLSAAIRDDAVWVNRSAAFDDAQVRIGASLTREDIWDMLDP |
| | | FFGSREAYLTPAKKKKEDEDSSEGTGEEKAKDLVQKAGQWLSSRFG |
| | | TGKGANFDAMAEVYSKISEWAGTAQEGVSGKEGIKNLADALAAFS |
| | | PVSQNLEGVLKLISGPGYKSATRNLLGELDSLPVVSRDHLSALHEKAA |
| | | EDTVKCKESTGTKGRRPYADAILNDVEKRCGFTYLTDSDNRSVSILDT |
| | | SEFPSDYKWGTARHSEFAVILDHAARRISVAHSWIKLAEAERDRCEE |
| | | DAAKVYDLPDKVKEWLDTFCSNRSDISGAQGEGYRIRRKAIEGWKE |
| | | WASWGRSSCITAEDRVAAARALQDDPEIDKFGDIQLFEILAQDEAL |
| | | CVWHKDGDVAKSPDAQMLIDYVLASDAESKKRRFKVPAYRHPDAL |
| | | LHPIFCDFGNSRWDITYDIHGARGKKKAKRGSKKEEAMPRGVAMK |
| | | LWTGSDVLSVSLRWQSKKLAADLALDQEAEEVTDTAAVSRADRLG |
| | | RAAAGIDRGAGVTIAGLFEEAHWNGRLQAPRQQLEAIAAVRDNQK |
| | | LSSEERERRIAFMKDRIRWLVTFSAKLRPQGPWHSYAPTQGLQSDP |
| | | KYWPHSEINKKRKGQAKLILSRLPGLRILSVDLGHRFAAACAVWET |
| | | MSSEAIQEACRLANHQLPAPADLYLHLKRTVQKNLIDGEKTVEESTV |
| | | YRRIGADRLPDGTAHPAPWARLDRQFLIKLQGEEKVREASNEEVW |
| | | QVHLMESALGLSFPLIDRLVYAGWGGTEKQAARLEALREKGWKPT |
| | | GTPADQDEEGGGYKPSLAVDELMFSAVRTLRLALKYHGDRARIAFA |
| | | LTADYKPMPGDTRYYFSEAKDRSSGADAAEREAKHKDYLLDMLLL |
| | | WHDLAFSRKWRDEEAKELWNLHIAALPGYQAPAAPIQEEAGQGRK |
| | | KAREEARAKMTPAAEALLADGTLREKLHGLWKERWEKDDAQWKK |
| | | HLRWMKDGILPRGGRAATPSIRYVGGLSLTRLATLTEFRRKVQVGFY |
| | | TRLFPSGEKREIKEAFGQTALDALERLREQRVKQLASRIAEAALGAGR |
| | | VSRTALKQDPKRPEARVDAACHAVIIENLEHYRPEETRTRRENRGLM |
| | | NWASSKVKKYLSEACQLHGLFLREVPAGYTSRQDSRTGAPGMRCQ |
| | | DVTVKTFLNSPFWQKQCVQAQKNKSTARDRFLCALKEAVAQGGM |
| | | EEEKKMGPIRVPVPGGEVFVSADAASPAAKGLQADLNAAANIGLRA |
| | | LLDPDWPGKWWYVPCDRKTAYPAKEKVEGSAAVDVKQALPFVLPE |
| | | EKENKGKTKGGKKGKGEVMNLWRDVSAEPLMTGQWLDYTAYRK |
| | | EVENRVIQVLTAQLKARNPLRFGNLGDEEEIPY |
| 138 | Cas12 | MAIRSIKLKLKTHTGPEAQNLRKGIWRTHRLLNEGVAYYMKMLLLF |
| | | RQESTGERPKEELQEELICHIREQQQRNQADKNTQALPLDKALEALR |
| | | QLYELLVPSSVGQSGDAQIISRKFLSPLVDPNSEGGKGTSKAGAKPT |
| | | WQKKKEANDPTWEQDYEKWKKRREEDPTASVITTLEEYGIRPIFPLY |
| | | TNTVTDIAWLPLQSNQFVRTWDRDMLQQAIERLLSWESWNKRVQ |
| | | EEYAKLKEKMAQLNEQLEGGQEWISLLEQYEENRERELRENMTAA |
| | | NDKYRITKRQMKGWNELYELWSTFPASASHEQYKEALKRVQQRLR |
| | | GRFGDAHFFQYLMEEKNRLIWKGNPQRIHYFVARNELTKRLEEAKQ |
| | | SATMTLPNARKHPLWVRFDARGGNLQDYYLTAEADKPRSRRFVTF |
| | | SQLIWPSESGWMEKKDVEVELALSRQFYQQVKLLKNDKGKQKIEFK |
| | | DKGSGSTFNGHLGGAKLQLERGDLEKEEKNFEDGEIGSVYLNVVIDF |
| | | EPLQEVKNGRVQAPYGQVLQLIRRPNEFPKVTTYKSEQLVEWIKASP |
| | | QHSAGVESLASGFRVMSIDLGLRAAAATSIFSVEESSDKNAADFSYW |
| | | lEGTPLVAVHQRSYMLRLPGEQVEKQVMEKRDERFQLHQRVKFQI |
| | | RVLAQIMRMANKQYGDRWDELDSLKQAVEQKKSPLDQTDRTFWE |
| | | GIVCDLTKVLPRNEADWEQAVVQIHRKAEEYVGKAVQAWRKRFAA |
| | | DERKGIAGLSMWNIEELEGLRKLLISWSRRTRNPQEVNRFERGHTS |
| | | HQRLLTHIQNVKEDRLKQLSHAIVMTALGYVYDERKQEWCAEYPAC |
| | | QVILFENLSQYRSNLDRSTKENSTLMKWAHRSIPKYVHMQAEPYGI |
| | | QIGDVRAEYSSRFYAKTGTPGIRCKKVRGQDLQGRRFENLQKRLVN |
| | | EQFLTEEQVKQLRPGDIVPDDSGELFMTLTDGSGSKEVVFLQADINA |
| | | AHNLQKRFWQRYNELFKVSCRVIVRDEEEYLVPKTKSVQAKLGKGLF |
| | | VKKSDTAWKDVYVWDSQAKLKGKTTFTEESESPEQLEDFQEIIEEAE |
| | | EAKGTYRTLFRDPSGVFFPESVWYPQKDFWGEVKRKLYGKLRERFL |
| | | TKAR |
| 139 | Cas12 | MPVRSINLKIVISRNTQGEKSRQSIWTTHAAVNDAVRYYEEQLLIMR |
| | | GLGYHISDKDVVSKESIQQERLSRIRRAQLENGLPEPLGTDAELNSLV |
| | | RKFYEFIVPSSVKEDGNAQQANGFLSPLTDPISIGYLSIFEKLGTIPDW |
| | | VGQLKAGDPQAVENAKKWSATSAGIKRLSETGAPPKWKKLFLTGD |
| | | PSWPQSFSEDIDKKIKEIEGAPKVICQLMEMGVLPLFPAYFADKLEGS |
| | | DGSLSRWDRLAFRLAVGHMLSWESWCIKSAEDHFERKRRVESFSEK |
| | | HTTPSLIICFETLEKYQKERQEKELGQNRSLPMQRPFRITRRQIRGWE |
| | | DLRDKWLKNTTRTYDSLKSIASKEQTKKGGRFGDPHLFLWLAKPEN |
| | | HAVWDADEDALSIFAKMNAMRGLLERSRETAYMTLPDPIEHPRSIQ |
| | | WEAEGGSNFKNYVITHSPVEGLHVQLPLLCKSESGKLIDQTFEFPLAP |
| | | SDQFKVAQISKTKSEVTITHQSVLDEEYRSKVGAADLLMDWPYLKN |
| | | RRFESVEHGDIGPVFLKLSLDIERILPDGWTPKRPQAISHFSSASGNS |
| | | KHKLSVVSGLRVLSVDLGIRSFGACSVFELSEHKPTSGMSFEIEGLNL |
| | | WANHERSFMLNLPDEDVGNKGRQLQKTKDAELRAMRRVLGRYRK |
| | | IYALAGIDPEDRKDILELLCQDQDIFEFERTIYKGLVTSTSVSQPLWEG |
| | | KIKESLKALRNAFGRKVREWRRANRLNSNLKYAGKTMWAIQHLED |
| | | TRRFLHSWSHLGRFSGEIRRADRVKRGVFATRLLQHLDSVKRDRLKT |
| | | GADLLVQSARGFLRDNQGNWKKSYAPCQVILFEDLSRYLMQTDRP |
| | | RRENSQLMKWSHRSIPLEVAMQGELYGIHVCDTSAAFSSRYHARLA |
| | | TPGIRCHALRKEDLSNQFLIESLQKENPDIDFGICKAGDLIPRGGGEIF |
| | | VSCDGNGGISRIHADINAAQNLQRRFWLRHGEAIRIPARKITLKGDEI |
| | | WVPRSIGKRLQGAMSGCGYLIPTGHESGSCRWERITASKWESISRSS |
| | | VAQKEEVNEDLLDIALLEEEALELSNEYTTFFRDPSGITLPSDLWFPM |
| | | KTFWGMTRAKIKSAIKQ |
| 140 | Cas12 | MTVKSIKVKLRLDNMPEIRAGLWKLHTEVNAGVRYYTEWLSLLRQE |
| | | NLYRRSPNGDGEQECYKTAEECKVELLERLRARQVENGHRDPAGSD |
| | | DELLQLARQLYELLVPQAIGAKGDAQQIARKFLSPLADKDAVGGLGI |
| | | AKAGNKPRWVRMRDAGEPGWEEEKAKAEARKSTDRTADVLRALA |
| | | DFGLKPLMRVYTDSDMSSVQWKPLRKGQAVRTWDRDMFQQAIE |
| | | RMMSWESWNQRVGEAYAKLVEQKSRFEQKNFVGQEHLVQLVNQ |
| | | LQQDMKEASHGLESKEQTAHYLTGRALRGSDKVFEKWEKLDPDAP |
| | | FDLYDTEIKNVQRRNTRRFGSHDLFAKLAEPKYQALWREDASFLTRY |
| | | AAYNSILRKLNHAKMFATFTLPDATAHPIWTRFDKLGGNLHQYTFLF |
| | | NEFGEGRHAIRFQKLLTIEHGVAKEVDDVTVPISMSAQLDDLLPGES |
| | | NEPTELSFRDHGTDQHFTGEFGGAKIQYRRDQLDHVHRRRGARDV |
| | | YLNLSVRVQSQSEARGERRPPYAAVFRLVGDTHRAFAHFDKLSNYL |
| | | AEHPDDGKLGSEGLLSGLRVMSVDLGLRTSASISIFRVARKDELKPNS |
| | | EGRVPFFFPIKGNDNLVAVHERSQLLKLPGETESKDLRAIREERQRIL |
| | | RQLRTQLAYLRLLVRCGSEDVGRRERSWAKLIEQSVDAANHMTPD |
| | | WREAFEGELQKLKSLYGICGDREWTEAVYESVRRVWRHMGKQVR |
| | | DWRKDVRSGERPKIRGYQKDVVGGNSIEQIEYLERQYKFLKSWSFF |
| | | GKVSGQVIRAEKGSRFATTLREHIDHAKEDRLKKLADRIIMEALGYVY |
| | | ALDAERGKGTWVAKYPPCQLILLEELSEYRFNNDRPPSENNQLMQ |
| | | WSHRGVFQELLNQAQVHDLLVGTMYAAFSSRFDARTGAPGIRCRR |
| | | VPARCAREQNPEPFPWWLNKFVAEHKLDGCPLRADDLIPTGEGEFF |
| | | VSPFSAEEGDFHQIHADLNAAQNLQRRLWSDFDISQIRLRCDWGE |
| | | VDGEPVLIPRLTGKRTADSYGNKVFYTNTGVTYYERERGKKRRKAFA |
| | | QEELSEEEAELLVEADEAREKSVVLMRDPSGIINRGDWTRQKEFWS |
| | | MVNQRIEGYLVKQIRSRVCLPESACENTGDI |
| 141 | Cas12 | MKNFQDFTNLYELSKTLRFELKPIGGTKKLIEEKNILKLDKKKRENYEK |
| | | VKPYFNKIHQEFINFALRNPNFDFSQFEEKYLNWLKDKKNKDLLKEK |
| | | ESIDKIFLEKIGKLFENSVKDFLKENGFESIVKEEDQNLKFFRRKEIFEVL |
| | | QEKYGSELETQMVNKDGEIKSIFNGWEKWLGYFDKFFNTRDNFYKT |
| | | DGTSTAIATRIIKDNLKIFLENIVAFGKIKNKKIDFSEVEKNFSVSIDTFF |
| | | EINNFNNCFLQDGIDFYNKVIGGETLENGEKLKGLNEIINKYRQDTGE |
| | | KIPYFKKLQKQILSEKDGVFIDKIEDDGGFYEVLKNFYKNAAEKEGFLK |
| | | NIFENFYTISDKNLEKIYFNKIAFNTISHKFGSALEFERILYEEMKKEKA |
| | | DGIKFEKKENKYKFPDFIQIIFIKRSLENYDSENLFWKERYYKSEENVD |
| | | GFLEKNNNNLWGQFCKILNFEFLNILKRRIIDEAGEEYEVGFEISKNIL |
| | | GEKLENFELNQENKGIIKDFADYSLALYSFGKYFAVEKGRNWDLNIDI |
| | | SDDFYGGEDGYIEKFYNTGYDEIVKPYNLMRNYISKKPWEDNKKWK |
| | | INFETSSLLSGWDKNLESNGSYIFQKGNKYYLGIINGSKPAKEILEKLYS |
| | | GDGEKIKRFIYDFQKPDNKNTPRMFIRSKKDSFSPAVEKYNLPINDIL |
| | | EIYDNGLFKTENKGNPNYKESLRKLIDYFKLGFSRHESFKHFNFVWK |
| | | DSKSYENIADFYRDVEKSCYKIDFEFLNFEELKKLTFEKHLYLFQIYNKD |
| | | FELDESLQEKGYNFKGEGQKNIHTKYFEALFLEENISRKSGAVFKLSG |
| | | GGEVFFRKKSIKAKKEKRNSVEVIKNKRYTECKYFLHFPIQVNFKEEIS |
| | | GNFNQEINKFLANNPDINVIGIDRGEKHLAYFSVINQKGEILESGSFN |
| | | KIENYNKNGEKLLFPEREIKEIHKDGSLIDLELVETGRKVDYVDYKLLLE |
| | | YKERKRLLQRQSWKEVEQIKDLKKGYISALVRKIADLIIKHNAIVIFEDL |
| | | NFRFKQIRGGIEKSIYQQLEKALIDKLNFLVNKNEINLEKAGSILKAYQ |
| | | LTVPVDSLKEIGKQTGVIFYTEAAYTSKIDPITGWRPNLYLKKNNSKIN |
| | | KENILKFDNIVFNSKENRFEFTYDLKKFFGKDSKFPAKTVNTVCSCVE |
| | | RFKWNRNLNNNKGGYIHYENLTDGKLANKEQKEDEFSNFKELFEKY |
| | | FIDINGNILEQIKNLDTKNNEKFFSSFIDLFTLVCQIRNTNQNAKGDE |
| | | NDFILSPVEPFFDSRKSQNFGKSLPKNGDENGAFNIARKGLIILNRISE |
| | | NPEKPDLLIFNADWDNFARNI |
| 142 | Cas12 | MAQAEAPRRLRAYKFALDPTEAQLREFEQHAGSARWAYNHANAIL |
| | | SRYSDTLRNRWNAWIAQHHGLSREQLYALPDRERTAIQAAARAAV |
| | | KAENAQLAAELRIIDDHRKRVTHKGKPSVEPGEQPAEDAPERAYQL |
| | | WRERVELARLHAEDPQAYRAERKRILDEIRPLVNATKRKLIEQGAYR |
| | | PTAMDISTLWREIRDLPPDEGGSPWWPEVSIYAFTSGFAHAETAW |
| | | KNYLESLAGRRAGRPVGKPRFKKKRRSRRSFTLYGSVKLVTYRRIQVP |
| | | SIGSVRLHGSAKRLHRALERRGGIIKSITISQGGHRWYASVLVDELDIT |
| | | PGRETQRGPSRRQRDRGAVGVDLGVHHLVALSDPNEKTLDNPRHL |
| | | RKARKRLLKAQRAMSRRRGPDKRTGQEPSRRWVKARNRVARLHH |
| | | ELAVRRAGHLHEITKRLATSYELVAIEDLNVAGMTRSARGTIDQPGR |
| | | GVRAKAGLNRSILDTSPAEFRRQLQYKASWYGATVAVIDRWAPTSR |
| | | TCSSCGAVKAKLSLAERTFFCEHCGMELDRDINAARNILAFAQSAYP |
| | | GEGKALNACGGSVSPGSQSVVQAGADEAGRPARKPRRSSRGSDPP |
| | | ATPTTRA |
| 143 | Cas12 | MKRIAKFRHDKPVKREAWSKGYRVHKNRIINKVTRSIKYPLVVKDE |
| | | WKKRLIDDAAHDYRWLVGPINYSDWCRDPNQYSILEFWIDFLCVG |
| | | GVFQSSHSNICRLAIQLSGGSVFEQEWKDLSPFVRANLIQGIKPAEFI |
| | | GFLTAEFRSSSNPKNFISKFFEGSNEDLESLTNEFASIVDFIKAKDISLLR |
| | | KSLPSCKKIAPNLWEKAVGSHSTNELLKLLTKYTRVMLVAEPSHSDR |
| | | VFSQTVLQSNDQDDPELTGPLPSHKVGKASYLFIPEFIREVNLDKISKL |
| | | DLSAKSKLAVEQVKKLSELTSDFKQIENQSEAYFGLSTSFNELSNFLGI |
| | | LIRTLRNAPEAILKDQIALCAPLDKDILKITLDWLCDRAQALPENPRFE |
| | | TNWAEYRSYLGGKIKSWFSNYENFFEIPQAASSQQNNNREKKLGNR |
| | | SAIRALNLKKEAFEKARETFKGDKGTLEKIDLAYRLLGSISPEVLQCDE |
| | | GLKLYQQFNDELLVLNETINQKFQDAKRDIKAKKEKESFEKLQRNLSS |
| | | PLPRIPEFFGERAKKGYQKARVSPKLARHLLECLNDWLARFAKVEES |
| | | AFSEKEFQRILDWLRTSDFLPVFIRKSKDPPSWLRYIARVATGKYYFW |
| | | VSEYSRKRVQIIDKPIAQNPLKELISWFLLNKDAFSRDNELFKGLSSK |
| | | MVTLARIMAGILRDRGEGLKELQAMTSKLDNIGLLHPSFSVPVTDSL |
| | | KDAAFYRAFFSELEGLLNIGRSRLIIERITLQSQQSKNKKTRRPLMPEP |
| | | FINEDKEVFLAFPKFETKNKVKGTRVVYNSPDEVNWLLSPIRSSKGQL |
| | | SFMFRCLSEDAKIMTTSGGCSYIVEFKKLLEAQEEVLSIHDCDIIPRAF |
| | | VSIPFTLERESEETKPDWKPNRFMGVDIGEYAVAYCVIEKGTDSIEIL |
| | | DCGIVRNGAHRVLKEKVDRLKRRQRSMTFGAMDTSIAAARESLVG |
| | | NYRNRLHAIALKHGAKLVYEYEVSAFESGGNRIKKVYETLKKSDCTGE |
| | | TEADKNARKHIWGETNAVGDQIGAGWTSQTCAKCGRSFGADLKA |
| | | GNFGVAVPVPEKVEDSKGHYAYHEFPFEDGLKVRGFLKPNKIISDQK |
| | | ELAKAVHAYMRPPLVALGKRKLPKNARYRRGNSSLFRCPFSDCGFT |
| | | ADADIQAAYNIAVKQLYKPKKGYPKERKWQDFVILKPKEPSKLFDKQ |
| | | FYRPN |
| 144 | Cas12 | MAQAEAPRRLRAYKFALDPTEAQLREFEQHAGSARWAYNHANAIL |
| | | SRYSDTLRNRWNAWIAQHHGLSREQLYALPDRERTAIQAAARAAV |
| | | KAENAQLAAELRIIDDHRKRVTHKGKPSVEPGEQPAEDAPERAYQL |
| | | WRERVELARLHAEDPQAYRAERKRILDEIRPLVNATKRKLIEQGAYR |
| | | PTAMDISTLWREIRDLPPDEGGSPWWPEVSIYAFTSGFAHAETAW |
| | | KNYLESLAGRRAGRPVGKPRFKKKRRSRRSFTLYGSVKLVTYRRIQVP |
| | | SIGSVRLHGSAKRLHRALERRGGIIKSITISQGGHRWYASVLVDELDIT |
| | | PGRETQRGPSRRQRDRGAVGVDLGVHHLVALSDPNEKTLDNPRHL |
| | | RKARKRLLKAQRAMSRRRGPDKRTGQEPSRRWVKARNRVARLHH |
| | | ELAVRRAGHLHEITKRLATSYELVAIEDLNVAGMTRSARGTIDQPGR |
| | | GVRAKAGLNRSILDTSPAEFRRQLQYKASWYGATVAVIDRWAPTSR |
| | | TCSSCGAVKAKLSLAERTFFCEHCGMELDRDINAARNILAFAQSAYP |
| | | GEGKALNACGGSVSPGSQSVVQAGADEAGRPARKPRRSSRGSDPP |
| | | ATPTTRA |
| 145 | Cas12 | MAIRSIKLKLKTHTGPEAQNLRKGIWRTHRLLNEGVAYYMKMLLLF |
| | | RQESTGERPKEELQEELICHIREQQQRNQADKNTQALPLDKALEALR |
| | | QLYELLVPSSVGQSGDAQIISRKFLSPLVDPNSEGGKGTSKAGAKPT |
| | | WQKKKEANDPTWEQDYEKWKKRREEDPTASVITTLEEYGIRPIFPLY |
| | | TNTVTDIAWLPLQSNQFVRTWDRDMLQQAIERLLSWESWNKRVQ |
| | | EEYAKLKEKMAQLNEQLEGGQEWISLLEQYEENRERELRENMTAA |
| | | NDKYRITKRQMKGWNELYELWSTFPASASHEQYKEALKRVQQRLR |
| | | GRFGDAHFFQYLMEEKNRLIWKGNPQRIHYFVARNELTKRLEEAKQ |
| | | SATMTLPNARKHPLWVRFDARGGNLQDYYLTAEADKPRSRRFVTF |
| | | SQLIWPSESGWMEKKDVEVELALSRQFYQQVKLLKNDKGKQKIEFK |
| | | DKGSGSTFNGHLGGAKLQLERGDLEKEEKNFEDGEIGSVYLNVVIDF |
| | | EPLQEVKNGRVQAPYGQVLQLIRRPNEFPKVTTYKSEQLVEWIKASP |
| | | QHSAGVESLASGFRVMSIDLGLRAAAATSIFSVEESSDKNAADFSYW |
| | | IEGTPLVAVHQRSYMLRLPGEQVEKQVMEKRDERFQLHQRVKFQI |
| | | RVLAQIMRMANKQYGDRWDELDSLKQAVEQKKSPLDQTDRTFWE |
| | | GIVCDLTKVLPRNEADWEQAVVQIHRKAEEYVGKAVQAWRKRFAA |
| | | DERKGIAGLSMWNIEELEGLRKLLISWSRRTRNPQEVNRFERGHTS |
| | | HQRLLTHIQNVKEDRLKQLSHAIVMTALGYVYDERKQEWCAEYPAC |
| | | QVILFENLSQYRSNLDRSTKENSTLMKWAHRSIPKYVHMQAEPYGI |
| | | QIGDVRAEYSSRFYAKTGTPGIRCKKVRGQDLQGRRFENLQKRLVN |
| | | EQFLTEEQVKQLRPGDIVPDDSGELFMTLTDGSGSKEVVFLQADINA |
| | | AHNLQKRFWQRYNELFKVSCRVIVRDEEEYLVPKTKSVQAKLGKGLF |
| | | VKKSDTAWKDVYVWDSQAKLKGKTTFTEESESPEQLEDFQEIIEEAE |
| | | EAKGTYRTLFRDPSGVFFPESVWYPQKDFWGEVKRKLYGKLRERFL |
| | | TKAR |
| 146 | Cas12 | MVVRSIKLKMKTNSGTDSIYLRKALWRTHRLINEGIVYYMSLLTLYR |
| | | QDTLGDRTKEEIQSELIKKIREQQRNNGLSEELGSDQEILSLLRHLYELI |
| | | IPSINGESGDANQLGNKFLYPLVDPNSQSGKGTSNAGRKSKWKRM |
| | | KEEGNPDWEVEFKKDEERKANDPTIKVFDHLKKYSLLPLFPLFTNNQ |
| | | KDIEWLPMGKRQSVRKWDKDMFIQAIERLLSWESWNRRLGAERE |
| | | KLEEKIENFYKEHLSGGQIWIEKIREFERVRDRELGETSFSSNDGYLITS |
| | | RQIRGWDRVYEKWSKISESASKEELWRVVAEQQSKMREGFGDPKV |
| | | FSFLAELENMDIWRKHPERIYHIATYNGLLKKLSHTKAQATFTLPDAV |
| | | KHPLWIRYEAQGGTNLNLFKLEETTKKNCKVILSKIIWPTEDGWFEK |
| | | ENVEVDLAPSKQFYRQIKLQDHIKGKQEISFSDYSSGISLKGVLGGSRI |
| | | QFDRKYIENHQELLPSGDIGPVFFNLVIDLLPIQETRNGRLKSPIGKTL |
| | | KVVSSEFPKVIDYKPKELTEWINVSSVTGKVGVESITEGMRVMSIDL |
| | | GQRTSASVSIFEVVKELPKDKEKMLYYNIKDTELFALHKRSFLLNLPGE |
| | | EVTKRNRQKRKDRRKKLLFIRSQIRMLASVLKLETKNTPDERKKAINK |
| | | LVEIVDSYEWTESEKEIWNSEFEYLTNKAVFKQEIWRESLIKSHHRM |
| | | EPHVGQLVSEWRKSLNEGRRNLAGITMWNIEELEDTRRLLISWSKR |
| | | SRTPGEANRINNDEPFGAKLLEHIQNVKDDRLKQLANLIVMTALGYK |
| | | YDKEEKSRDKRWKEKYPACQVILFENLNRYLFSLDRSKRENSKLMK |
| | | WAHRSIPRTVWMQGEMFGLQVGDVRSEYSSRFHAKSGAPGIRCH |
| | | SLNDEDLKEESFKLKHLIETGFISEEEISSLKKGDIVPWPGGELFVTLSK |
| | | PYKKGKDIELTVIHADINAAQNLQKRFWQQNSEVYRIPCQLEKAGD |
| | | DEFFIPKSQTEIVKKYFGKGRFVKINDKKEVYNWEESEKMKIKTESTIT |
| | | LQDLEGFEDIFQTLELAQEQQKKYSTLFRDPSGYFFNEKNWRPQKEF |
| | | WSIVNNIIRSSLKKKILKNKVEV |
| 147 | Cas12 | MSNPNIPNISPNITLTRDDVVNLLMSSIAMEELGLAHIINAEGEKIQF |
| | | ALGTLQGASGPPATLQQVLEVNQSTQAMLDTIFRQEMMLDSKLKT |
| | | ATNIPTLRGPTGPVGPTGAPGGVISINGQTGVVTLDASNGVMPFM |
| | | REQSTSSLDDYKDPGIYXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXGATGATGATGATGPQEPRALREPRAPQARREPRGLLEPQVL |
| | | RGPQEPRALREPRAPRALREPRALRALRALQELRAPRALRGLQEPRV |
| | | LQGPRERQVRPEPRVLQGPRERQVRQGLQGLLEPRAKRERQVRPE |
| | | PREPQERQAPLVQQALLEPQALLGQAL |
| 148 | Cas12 | MNRIYQGRVSKVEIPNPGDKGKPWQPLPNWQDILWQHHELFQD |
| | | AVNYYLLALLSLARDSANPATPIRKRMDDPASEHQIWTSFRRRGQM |
| | | RSGMRDSVAKYLCPVKTEPTLDECFAAVLEGNKENPDVLDLALQELL |
| | | DECDGDGAIQQEGRSMLPRFCSPSYKGDFPQSAAAKDKKSAKEWL |
| | | PTFLHSAENASNLKLVRQRLKFELFANRDLTGRILDAVESRKRLTEML |
| | | GWLTERNPSLAAERERLQKIIAALPDTFNLPAWRGGSVNKEALKQR |
| | | FYAWLIFEHVESSRITFDILRDSFPTPKEKRKSVSETIQSEKPSLELSDD |
| | | PIKLARGTRGYVFRAFTSLPCWGADNANLLAWKEFDVAAFKEALKA |
| | | LHQVESKSEERNKERERLLQRHSVMRGSIKWKPSPESEEKEPDVLAG |
| | | DPRIERLEKLLKTELASEYEMSEGQTVEYGLQPRTIRGFRDLRKEWN |
| | | KIVKPGEPFTEQKKGNLVTALRSYQIENPNVIGSVRLYEALLEKGNWL |
| | | VWQEADSATVEKWAKQKFASDPLEALTEERQLLRDIERLKQPIRFTP |
| | | ADAVHSRRQFYLAEKGDLSVKNRFDPQNQ.TLQVPIAIKNDDHWKQ |
| | | QFVKIHFSAPRAVRDQLIDETMEESKEARWQQPMMEALGLSLKLT |
| | | KNNAEVSLSECTAVSLMPEELASGDRRILLNFPITLETAPMVKALGKA |
| | | SLWDGQFAAYGDDNFYLRWPKDKWPAEKESNAWYRRLTEFSLLSV |
| | | DLGQRDAGAFAVIGATTGKPTRPASRFIGEANGQSWHASVRATGV |
| | | LRLPGEDAHVFRSGQWQEELYGERGRSADATEWTEAKRICETLGLK |
| | | FDEILGGDPQWFSFPELNDRLLFAVRRAQTRLARLQSWSWMIADE |
| | | TRRENIREAILAADDDELSLKAAAEKSLWPVLAEKLASEVNRLRNLIP |
| | | QQLVLLANRILPLRGRRWEWIKRDDTSGCHVLRETNPGTDTTSKKV |
| | | CGQRGLSMKRLEQLDELRRRFQSLNRALMQTPGQTAGLGKSKRGI |
| | | ELPDPCPDLLDKTEQLREQRVNQTAHLILAQALGVRLRTPQKGDAL |
| | | REQNNVHGEYETFRLPVDFIVLEDLSRYLSSQGRGRNENSRLMKWC |
| | | HRAILLKLKQLCESYGLKILETNAAYSSRFCSRTGVAGFRAVELTPDAR |
| | | KEFRWRKHLNRLEKAASGEIKLDREARAESEHVKRLFDMLDQLNAG |
| | | RKEAGKPLRTLLAPIAGGQIFIPMQGHATQADINAAINLGLRAIAAP |
| | | DCHAIHVRIRTERKDKTLRVRTGSNREKVRWEGKNPEIQMNKAADL |
| | | ASLTGDRQPNFFPDPSAIACYDRAKIEGVQLPFASGRGLWGTMNIE |
| | | LQWTRVNQLNNDRAEKKWNLTRSLPEQSSEEDYIPM |
| 149 | Cas12 | MAVKSIKVKLMLGHLPEIREGLWHLHEAVNLGVRYYTEWLALLRQG |
| | | NLYRRGKDGAQECYMTAEQCRQELLVRLRDRQKRNGHTGDPGTD |
| | | EELLGVARRLYELLVPQSVGKKGQAQMLASGFLSPLADPKSEGGKG |
| | | TSKSGRKPAWMGMKEAGDSRWVEAKARYEANKAKDPTKQVIASL |
| | | EMYGLRPLFDVFTETYKTIRWMPLGKHQGVRAWDRDMFQQSLER |
| | | LMSWESWNERVGAEFARLVDRRDRFREKHFTGQEHLVALAQRLE |
| | | QEMKEASPGFESKSSQAHRITKRALRGADGIIDDWLKLSEGEPVDRF |
| | | DEILRKRQAQNPRRFGSHDLFLKLAEPVFQPLWREDPSFLSRWASY |
| | | NEVLNKLEDAKQFATFTLPSPCSNPVWARFENAEGTNIFKYDFLFDH |
| | | FGKGRHGVRFQRMIVMRDGVPTEVEGIVVPIAPSRQLDALAPNDA |
| | | ASPIDVFVGDPAAPGAFRGQFGGAKIQYRRSALVRKGRREEKAYLC |
| | | GFRLPSQRRTGTPADDAGEVFLNLSLRVESQSEQAGRRNPPYAAVF |
| | | HISDQTRRVIVRYGEIERYLAEHPDTGIPGSRGLTSGLRVMSVDLGLR |
| | | TSAAISVFRVAHRDELTPDAHGRQPFFFPIHGMDHLVALHERSHLIR |
| | | LPGETESKKVRSIREQRLDRLNRLRSQMASLRLLVRTGVLDEQKRDR |
| | | NWERLQSSMERGGERMPSDWWDLFQAQVRYLAQHRDASGEAW |
| | | GRMVQAAVRTLWRQLAKQVRDWRKEVRRNADKVKIRGIARDVP |
| | | GGHSLAQLDYLERQYRFLRSWSAFSVQAGQVVRAERDSRFAVALRE |
| | | HIDNGKKDRLKKLADRILMEALGYVYVTDGRRAGQWQAVYPPCQL |
| | | VLLEELSEYRFSNDRPPSENSQLMVWSHRGVLEELIHQAQVHDVLV |
| | | GTIPAAFSSRFDARTGAPGIRCRRVPSIPLKDAPSIPIWLSHYLKQTER |
| | | DAAALRPGELIPTGDGEFLVTPAGRGASGVRVVHADINAAHNLQRR |
| | | LWENFDLSDIRVRCDRREGKDGTVVLIPRLTNQRVKERYSGVIFTSE |
| | | DGVSFTVGDAKTRRRSSASQGEGDDLSDEEQELLAEADDARERSVV |
| | | LFRDPSGFVNGGRWTAQRAFWGMVHNRIETLLAERFSVSGAAEKV |
| | | RG |
| 150 | Cas12 | MGLVDTAGGFLIPAALDPAlLLSGDGSTNPIRQVXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXPSRCRCLAFGRSSSAIRRPAASSSEGVKSL |
| | | RSVFSERSALARSTTSAIVWHTCTGMRSHPSSRAASSRRWPNTSRLS |
| | | GVTPIGCSKRQGDSPCGCCCGAHLVVAQRCVAFCCHDRGVTEKLLY |
| | | SSQVVGAAVGAGGVSVPQGVHGRVTG |
| 151 | Cas12 | MRRQHHGGQNARDWRRKVAAAALRQKESVFTYKFGLSVNDGDF |
| | | DFDAAARTYDITEGIERGSLIGLVCAVHLSGFRLFSKVAETRQFLNRS |
| | | RYPENEFAQALAAHTEIENPSVTVQSIESVFVTPPRKQDGVARLWSA |
| | | DELAKRLFQTWNNRSPREGERNHPELLLAQGIARAVTKAFSGWKEL |
| | | ADNAVHALTCADNYLATLGNRFPKLSDLPPLTAGSTQTGTLAFDPES |
| | | PFLNMTGNEDIWLHQVVAVCAGRLKRYMPEIDPSSRKFASRLTDSI |
| | | VSSQNNGLSWLFGNGLRFLRQSSIAQIAETLSVSQNEHRRVEQLKEF |
| | | ADAIPVNPFFATDGYAEFRGSVGGKISSWVSNYWKRICELTVLHSQ |
| | | PPDITIPEGLLASENATLFSGQHTAAAGLVALSARLPSQVRDAGKALF |
| | | VLSGDGVPRADDIATVEDVAGELAELTGQLAMLDNRIQQEIERAQD |
| | | ANDEGRVGSLASLRPNPTKELKEPPKLNRISGGTADAAGELARLETSL |
| | | NDLIRARREHFYRLAEWTGNTASLDPLPALAERERKALTDRGMDPT |
| | | LAEADEYALRRLLHRIAGMARRLSPNEAKRVRETMTPLFLKKREANL |
| | | YFHNRAGALYRHPFSNSRHQPYSIDLNRARATDWLAWLEERAREM |
| | | LGLLGSGAPANHEYLRDLLSIETFVFTTRLSGLPAQVPGYLAKPKSDLT |
| | | NIPPLLAAQLDVDEVSRDVALRAFNLFNSAINGLSFRAFRDSFIVRTK |
| | | FLRLGHDELFYVPKARAWKPPADYRSAKGKISKGLALPAVKRNEAGS |
| | | ILPRETTQGLSRAKFPEGSHALLSQAPHDWFVELDLRHDKMPQLAG |
| | | LPVKMNADGLKGWRARRRPTFRLAGPPSFKTWLDRALTSTAVKLG |
| | | DYTLILDQSFKQSLRVEDGEVRLSAEPAGIKAEIAVPVIDARPFPETEA |
| | | EALFDNIIGIDLGERRIGYAVFSLPALLKSGNPTRVKPTVVGSVAIPAF |
| | | RRLMAAVRRHRGSRQPNQKVSQTYSTALQQFRENVVGDVCNRID |
| | | TLCERYRAFPVLESSVANFETGANQLKLIYGTVLRRYTFSNVDAHKSA |
| | | RSAYWYSANRWQHPYLFVREWNKAQRTFTGSAKPLAIYPGVTIHP |
| | | AGTSQICHRCGRNALRALRNMPDRTIRVGKDGLIVLADSTIRLLERA |
| | | DYSDRELKTFKRRKQRPPLNMPVPEGARPRDQLERVLRRNMRQQP |
| | | QSEMSPDTTQARFTCVYTDCGFEGHADENAAVNIGRRFLERIDIEAS |
| | | SRT |
| 152 | Cas12 | MQEIKRINKIRRRLVKDSNTKKAGKTGPMKTLLVRVMTPDLRERLE |
| | | NLRKKPENIPQPISNTSRANLNKLLTDYTEMKKAILHVYWEEFQKDP |
| | | VGLMSRVAQPAPKNIDQRKLIPVKDGNERLTSSGFACSQCCQPLYV |
| | | YKLEQVNDKGKPHTNYFGRCNVSEHERLILLSPHKPEANDELVTYSL |
| | | GKFGQRALDFYSIHVTRESNHPVKPLEQIGGNSCASGPVGKALSDA |
| | | CMGAVASFLTKYQDIILEHQKVIKKNEKRLANLKDIASANGLAFPKIT |
| | | LPPQPHTKEGIEAYNNVVAQIVIWVNLNLWQKLKIGRDEAKPLQRL |
| | | KGFPSFPLVERQANEVDWWDMVCNVKKLINEKKEDGKVFWQNLA |
| | | GYKRQEALLPYLSSEEDRKKGKKFARYQFGDLLLHLEKKHGEDWGK |
| | | VYDEAWERIDKKVEGLSKHIKLEEERRSEDAQSKAALTDWLRAKASF |
| | | VIEGLKEADKDEFCRCELKLQKWYGDLRGKPFAIEAENSILDISGFSK |
| | | QYNCAFIWQKDGVKKLNLYLIINYFKGGKLRFKKIKPEAFEANRFYTV |
| | | INKKSGEIVPMEVNFNFDDPNLIILPLAFGKRQGREFIWNDLLSLETG |
| | | SLKLANGRVIEKTLYNRRTRQDEPALFVALTFERREVLDSSNIKPMNL |
| | | IGIDRGENIPAVIALTDPEGCPLSRFKDSLGNPTHILRIGESYKEKQRTI |
| | | QAAKEVEQRRAGGYSRKYASKAKNLADDMVRNTARDLLYYAVTQD |
| | | AMLIFENLSRGFGRQGKRTFMAERQYTRMEDWLTAKLAYEGLPSK |
| | | TYLSKTLAQYTSKTCSNCGFTITSADYDRVLEKLKKTATGWMTTING |
| | | KELKVEGQITYYNRYKRQNVVKDLSVELDRLSEESVNNDISSWTKGR |
| | | SGEALSLLKKRFSHRPVQEKFVCLNCGFETHADEQAALNIARSWLFL |
| | | RSQEYKKYQTNKTTGNTDKRAFVETWQSFYRKKLKEVWKPAV |
| 153 | Cas12 | MGKQSTNSSGLKATSGAPSIEKPVTTQRAYTLRLRGIEGDKTWRDSL |
| | | WATHEAINLGAKAFGDWLLTLRGGLEHTLADATVAGGKGKPARPP |
| | | SADERRDRRVLLALSWLTVEDERGAPKAPGLIVAYGDDCKSAKDSQ |
| | | DGRDRKVEDALRDILIKRSVAPGAVESWVNDCVVSLKARIRDDAVW |
| | | VNRSAAFDALRASWRGLTCPNARTVLEQFFGPVADWITLPARAAD |
| | | DGEGDTGGPTAAGSSNDTEFKLVARSFLSENFGTGLKSSKSDISDAL |
| | | MQATRSLAALRGGSPGSAVWSHLCTEFGLLAADDEARAKALRVRIG |
| | | WTSGRRSKGRLALATAASKASLAQVDIELLIKKLSEEAQEKLAGSTKL |
| | | PLPWVNDLMAALERSIGFGFVTERNLIDEFGVMLDHAARRVSIACS |
| | | WIKLAELERRQFEEDAKKLDQVRTQHADAAAFLDTLGCRRGSESGS |
| | | AGGASVVIRKRAVLGWNEVVGDWARQGCKSVQERIDAARLLQGE |
| | | LEKFGDIRLFEELAANDAQVVWQNAEGETDPTILARYSAGSVAKAN |
| | | QQRFKVPAYRHPDPLRHPVFGDFGKSRWKIDFAVHESDRAQSGGK |
| | | RVRSLDAAWQSDVRNMRMALWTGKRVEMVPLRWSSKRLSKDLG |
| | | ISDLSLKDPTVVTRGDRLGRAAVGPGDPVRVASVFAEDNWNGRLQ |
| | | APRVELNRVARLTQSGNLVQARRLRDRLSWLVSFSPKLAPSGPFIDY |
| | | AAAHGIEPHKKSGEYWPNSAMNKERKGHHSKLIYSRLPSLRVLSVDL |
| | | GHRFAAACAVWESMSAAQMRLAASKGKVVRGGIAEGALFIHIEST |
| | | VADGTVRTTVYRRIGEDALPDGSPHPAPWAKLERQFLIKLQGEETPS |
| | | RMAALDELVMIADWERALGYQPVTASSTKQSNGVAGLMGRTVRL |
| | | FMHASRRHFDRARIAHNLTAEHRTKPGGVPESLTEETRIELLIDTLAL |
| | | WHGLFAGDRWRDPRAQKEWETSGLPSLKLPGRGDDDETAFGGPG |
| | | RKAAMVVYRNELKPHAERMAHSDLSNLSKCWTDRWAQDDREWT |
| | | AKSGRLRSLRRWITPRGLRPVSGDSAQLIERKAAAALRARHVGGLS |
| | | MQRINTLTDLYRLLKSFKNRPEPSNLRKNIPEKGDARLIGFNQRLLDV |
| | | RDRLREQRVKQLASRIVEAALGIGRIKIRRVAAGSPRPTLRVDAACHA |
| | | VVTENLSNYQPAELQTRRENRQLMAWASSKVHKYLAESCQLHGLH |
| | | LREVQPNYTSRQCSRTGAAGLRGvAVSATDLLTNPWWMRDVAQA |
| | | KARIEKALKNGRNGAVADQLLVSAEAWALRIPERDRHDRKADESVK |
| | | AFHAERVILPRKGGDLFVAESARCNGKGHVPALQADLNAAANVGIR |
| | | ALLDPDWPGKWWWVPCVGGTYSPSPEKTSGAKVLEHIDQLPHEM |
| | | VDPPQVAPKSSAPQPKSSRAAKVKAKEFRAIENRWRTCSSELLSQGK |
| | | WIPSGEYWSKVEAAVCLVLNDQIT |
| 154 | Cas12 | MAAFQRSYTMNLKPATSEQDKFILWNRLFLTHWSVNEGAKIFGELF |
| | | LNLRGGLSPELDIFDLDKVKDDKKKKAFVMGRRRLLALGWLSVEDN |
| | | LSAGEHPFRIREIPVGRNMGKSQASTLLTEILKNKGIKDEAVIKEWID |
| | | DCTPSLIANIREDAVWINRASSFNSITPCPTKDEVWIVLSGLLGLRFLD |
| | | LSLEEVKGKETEYLYDLGEKETQSKSDPSKKARELFGNLFTQNPVLMK |
| | | NSRDKKDTFAKEFYLAFKEFKDYEKLKEKIESWRKEKEFPLIENPVAEK |
| | | YPPEVTFTGSPCTVSKRYRKLLVSLELWPSSQDENGNIPKTEKTEDKT |
| | | HNQVLLDYLLKACNEGNKGTQKHTPVWANNLKAELELKMNEIIRIG |
| | | ESSSTELQRLMIKMAARRISQTLSWIKINEQTKHDAYQKKNKAFKLL |
| | | SEIDKNGEACKWLENYELFRTDDSGGEEYHISLRAISCWKQILEEWQ |
| | | KNDSPKALREKVKEVQAEEEKFGDARLFEDLADDNARSVWLLPDG |
| | | NKTPDILNWWCEYRTAEIDESRFKIPCYCHPHPFKHPVYVEYGKSNP |
| | | KVIFAMKNNKVKKGHIEHGWNPQNPRSIALSLFNNGNRESSLVPFI |
| | | WESKRLWKDLGGEATQIGDIPRSDRMGLSGKRESVKPKAPFQKEV |
| | | WNARLQSDRRTLEKLEKYWNPESMKWIDDGKFLIQSKWFITFGPD |
| | | METAEGPWKLYLKEKYVDNKILGNRSKENQKRGYRAKKLLSGYPAG |
| | | MRILSVDLGHRYAASCAVWETITKKQITEELAYQPDNNSLFEHSCKTI |
| | | DKKIKNTVYRRIGEDSIDAPWAKLEKQFTIKLQGEDKSCYLLRSDEKE |
| | | LFRSILSKLSCLNNDTGHNILEMIENLLRIVKAKIYRQGILARISYSMTA |
| | | QYKPGKGGQKSPLSDEDKIHYLSENLAAWSAIMGNQEWNEDVISD |
| | | WYKTYISHLVSGPKPKEGNRKSDRDKIIEYFLPAARKLYDDNETRIKIH |
| | | DLFKELWDENNKQLSAVLKEIKKIILPKGIRYFDKNTDNPSKWKNNQ |
| | | SKLKQITHRGGLSMQRIVAIEEYYKLAKAYKNHPEPDDLTKNIPLPGD |
| | | NSSAGFNQRIRDTLERMKEQRVKQIASRIVESALGLGIEGYKKRPLTP |
| | | ENKPCQAIVIEDLSHYRPDELQTRRENRRLMQWSSSKVKKYLKEACE |
| | | MHDVRLVEISPEYTSRQDSRTGAAGLRCIDINIREFLKDSSRWQNKI |
| | | NTIQKKPANKKSNLDQYLIELNESLGNKYKDKVIPSDNFVRIPRKGGD |
| | | VFVSSSKESPVSKGIQADLNAAANIGLKALLDPDWAGAWWYILIEV |
| | | KSNHVIPYGEKYKGSECLRAWKFSGLENQVMKNNMNLWRDLQSQ |
| | | FSGEDKWMSYKEYNELTEKRVINILRERAGLELIKE |
| 155 | Cas12 | MEDYSGFVNIYSIQKTLRFELKPVGKTLEHIEKKGFLKKDKIRAEDYKA |
| | | VKKIIDKYHRAYIEEVFDSVLHQKKKKDKTRFSTQFIKEIKEFSELYYKT |
| | | EKNIPDKERLEALSEKLRKMLVGAFKGEFSEEVAEKYKNLFSKELIRNE |
| | | IEKFCETDEERKQVSNFKSFTTYFTGFHSNRQNIYSDEKKSTAIGYRII |
| | | HQNLPKFLDNLKIIESIQRRFKDFPWSDLKKNLKKIDKNIKLTEYFSID |
| | | GFVNVLNQKGIDAYNTILGGKSEESGEKIQGLNEYINLYRQKNNIDR |
| | | KNLPNVKILFKQILGDRETKSFIPEAFPDDQSVLNSITEFAKYLKLDKKK |
| | | KSIIAELKKFLSSFNRYELDGIYLANDNSLASISTFLFDDWSFIKKSVSFK |
| | | YDESVGDPKKKIKSPLKYEKEKEKWLKQKYYTISFLNDAIESYSKSQDE |
| | | KRVKIRLEAYFAEFKSKDDAKKQFDLLERIEEAYAIVEPLLGAEYPRDR |
| | | NLKADKKEVGKIKDFLDSIKSLQFFLKPLLSAEIFDEKDLGFYNQLEGY |
| | | YEEIDSIGHLYNKVRNYLTGKIYSKEKFKLNFENSTLLKGWDENREVA |
| | | NLCVIFREDQKYYLGVMDKENNTILSDIPKVKPNELFYEKMVYKLIPT |
| | | PHMQLPRIIFSSDNLSIYNPSKSILKIREAKSFKEGKNFKLKDCHKFIDF |
| | | YKESISKNEDWSRFDFKFSKTSSYENISEFYREVERQGYNLDFKKVSKF |
| | | YIDSLVEDGKLYLFQIYNKDFSIFSKGKPNLHTIYFRSLFSKENLKDVCL |
| | | KLNGEAEMFFRKKSINYDEKKKREGHHPELFEKLKYPILKDKRYSEDK |
| | | FQFHLPISLNFKSKERLNFNLKVNEFLKRNKDINIIGIDRGERNLLYLV |
| | | MINQKGEILKQTLLDSMQSGKGRPEINYKEKLQEKEIERDKARKSW |
| | | GTVENIKELKEGYLSIVIHQISKLMVENNAIVVLEDLNIGFKRGRQKVE |
| | | RQVYQKFEKMLIDKLNFLVFKENKPTEPGGVLKAYQLTDEFQSFEKL |
| | | SKQTGFLFYVPSWNTSKIDPRTGFIDFLHPAYENIEKAKQWINKFDSI |
| | | RFNSKMDWFEFTADTRKFSENLMLGKNRVWVICTTNVERYFTSKT |
| | | ANSSIQYNSIQITEKLKELFVDIPFSNGQDLKPEILRKNDAVFFKSLLFYI |
| | | KTTLSLRQNNGKKGEEEKDFILSPVVDSKGRFFNSLEASDDEPKDAD |
| | | ANGAYHIALKGLMNLLVLNETKEENLSRPKWKIKNKDWLEFVWER |
| | | NR |
| 156 | Cas12 | MKRIAKFRHDKPVKREAWSKGYRVHKNRIINKVTRSIKYPLVVKDE |
| | | WKKRLIDDAAHDYRWLVGPINYSDWCRDPNQYSILEFWIDFLCVG |
| | | GVFQSSHSNICRLAIQLSGGSVFEQEWKDLSPFVRANLIQGIKPAEFI |
| | | GFLTAEFRSSSNPKNFISKFFEGSNEDLESLTNEFASIVDFIKAKDISLLR |
| | | KSLPSCKKIAPNLWEKAVGSHSTNELLKLLTKYTRVMLVAEPSHSDR |
| | | VFSQTVLQSNDQDDPELTGPLPSHKVGKASYLFIPEFIREVNLDKISKL |
| | | DLSAKSKLAVEQVKKLSELTSDFKQIENQSEAYFGLSTSFNELSNFLGI |
| | | LIRTLRNAPEAILKDQIALCAPLDKDILKITLDWLCDRAQALPENPRFE |
| | | TNWAEYRSYLGGKIKSWFSNYENFFEIPQAASSQQNNNREKKLGNR |
| | | SAIRALNLKKEAFEKARETFKGDKGTLEKIDLAYRLLGSISPEVLQCDE |
| | | GLKLYQQFNDELLVLNETINQKFQDAKRDIKAKKEKESFEKLQRNLSS |
| | | PLPRIPEFFGERAKKGYQKARVSPKLARHLLECLNDWLARFAKVEES |
| | | AFSEKEFQRILDWLRTSDFLPVFIRKSKDPPSWLRYIARVATGKYYFW |
| | | VSEYSRKRVQJIDKPIAQNPLKELISWFLLNKDAFSRDNELFKGLSSK |
| | | MVTLARIMAGILRDRGEGLKELQAMTSKLDNIGLLHPSFSVPVTDSL |
| | | KDAAFYRAFFSELEGLLNIGRSRLIIERITLQSQQSKNKKTRRPLMPEP |
| | | FINEDKEVFLAFPKFETKNKVKGTRVVYNSPDEVNWLLSPIRSSKGQL |
| | | SFMFRCLSEDAKIMTTSGGCSYIVEFKKLLEAQEEVLSIHDCDIIPRAF |
| | | VSIPFTLERESEETKPDWKPNRFMGVDIGEYAVAYCVIEKGTDSIEIL |
| | | DCGIVRNGAHRVLKEKVDRLKRRQRSMTFGAMDTSIAAARESLVG |
| | | NYRNRLHAIALKHGAKLVYEYEVSAFESGGNRIKKVYETLKKSDCTGE |
| | | TEADKNARKHIWGETNAVGDQIGAGWTSQTCAKCGRSFGADLKA |
| | | GNFGVAVPVPEKVEDSKGHYAYHEFPFEDGLKVRGFLKPNKIISDQK |
| | | ELAKAVHAYMRPPLVALGKRKLPKNARYRRGNSSLFRCPFSDCGFT |
| | | ADADIQAAYNIAVKQLYKPKKGYPKERKWQDFVILKPKEPSKLFDKQ |
| | | FYRPN |
| 157 | Cas12 | MAIRSIKLKLKTHTGPEAQNLRKGIWRTHRLLNEGVAYYMKMLLLF |
| | | RQESTGERPKEELQEELICHIREQQQRNQADKNTQALPLDKALEALR |
| | | QLYELLVPSSVGQSGDAQIISRKFLSPLVDPNSEGGKGTSKAGAKPT |
| | | WQKKKEANDPTWEQDYEKWKKRREEDPTASVITTLEEYGIRPIFPLY |
| | | TNTVTDIAWLPLQSNQFVRTWDRDMLQQAIERLLSWESWNKRVQ |
| | | EEYAKLKEKMAQLNEQLEGGQEWISLLEQYEENRERELRENMTAA |
| | | NDKYRITKRQMKGWNELYELWSTFPASASHEQYKEALKRVQQRLR |
| | | GRFGDAHFFQYLMEEKNRLIWKGNPQRIHYFVARNELTKRLEEAKQ |
| | | SATMTLPNARKHPLWVRFDARGGNLQDYYLTAEADKPRSRRFVTF |
| | | SQLIWPSESGWMEKKDVEVELALSRQFYQQVKLLKNDKGKQKIEFK |
| | | DKGSGSTFNGHLGGAKLQLERGDLEKEEKNFEDGEIGSVYLNVVIDF |
| | | EPLQEVKNGRVQAPYGQVLQLIRRPNEFPKVTTYKSEQLVEWIKASP |
| | | QHSAGVESLASGFRVMSIDLGLRAAAATSIFSVEESSDKNAADFSYW |
| | | IEGTPLVAVHQRSYMLRLPGEQVEKQVMEKRDERFQLHQRVKFQI |
| | | RVLAQIMRMANKQYGDRWDELDSLKQAVEQKKSPLDQ.TDRTFWE |
| | | GIVCDLTKVLPRNEADWEQAVVQIHRKAEEYVGKAVQAWRKRFAA |
| | | DERKGIAGLSMWNIEELEGLRKLLISWSRRTRNPQEVNRFERGHTS |
| | | HQRLLTHIQNVKEDRLKQLSHAIVMTALGYVYDERKQEWCAEYPAC |
| | | QVILFENLSQYRSNLDRSTKENSTLMKWAHRSIPKYVHMQAEPYGI |
| | | QIGDVRAEYSSRFYAKTGTPGIRCKKVRGQDLQGRRFENLQKRLVN |
| | | EQFLTEEQVKQLRPGDIVPDDSGELFMTLTDGSGSKEVVFLQADINA |
| | | AHNLQKRFWQRYNELFKVSCRVIVRDEEEYLVPKTKSVQAKLGKGLF |
| | | VKKSDTAWKDVYVWDSQAKLKGKTTFTEESESPEQLEDFQEIIEEAE |
| | | EAKGTYRTLFRDPSGVFFPESVWYPQKDFWGEVKRKLYGKLRERFL |
| | | TKAR |
| 158 | Cas12 | MTHAKKIPFPVLKRSTLRKARQRIAAGSITAGERPFNSTVTRVVPVK |
| | | DPVSDQVWAVAREAAMTLRGFGQGSLFDMLIHLHADGFRLFPSG |
| | | REREAFFLKDLFDPTEFDDGARRAFGDVMPGFTANSLREILGAPARK |
| | | CGKVTSVEILLPRLSKGLGVKKSAAPPEVLSSLAAALCEAFPTWSLLTA |
| | | VDGGVGKVIDDVLRTHGSRLPSLEKAWSTNLPEVPKGLGVPTLAFD |
| | | DQAPAQSEQTPTGRFAGVVARYLAETFASNPEATAGDASKAVQAK |
| | | VTTPNGNALSWLFAVGRRAMCSTTLDELAIGLNITSPRGRHALSSLK |
| | | ERMMALPALSVLGERAYPDSRATLQGTVDSLIANYVNRLFELSSSAT |
| | | SIAQTKLILPAAIQGDTAVFDGMPFSAEDVGALFEQLPSEIAKLEHAV |
| | | KVLVGKERTSTLGYQKAVDDVDEFGVWASSVDAVIGQINARLKTLE |
| | | RAQEPLGKLMGDGKLKRLVNIHEPEGPAVEIIPVLDQELQDVLTSCR |
| | | TAFADLEARYPMTVAKAQRHAEAEVRNALELASRKEGGLSLASADV |
| | | PALAKRKILEPIISIARRSSPAMATAVLTECLRQKLIVKGTGSERSLRGY |
| | | VLSGEQVIYAHPLSRRRSIVRLDREGLQNFDALEFLDALQKDATQRT |
| | | NVRESLIVEMARQSLLLSALPDRIEIGAISWQTPSQNQHAPWANLR |
| | | PVNGTVGRSETIKSFTAVFHSRISGLLYRLNRQKFMEKYDLRCFIGSTL |
| | | LFSPKNADWAPPPQYRHGRFSALLARSDFPWEGAEGTHANAVRLA |
| | | KFLIDETRNATDLQQAIAAKALLAQLPHDWVVCCDFDGAPSYEGAF |
| | | VSAGEVSAWAKRSGYLLTPPRHFAGAFLEGFKSTKISPHGLTFERML |
| | | ERDGDSVIETGRRVTAAFPITQEVAPAAQPWKPRHLAGLDLGEAGL |
| | | GVCLKNLDNGHEQTLLLKTRKTRLLAHSAEHYRRKDQPRQVFRKQY |
| | | NQSSENAIKAAIGEVCGLIDNLIARYDAVPVFESQAAAARGSNRMV |
| | | ARVYAGVLQRYTYVVGNGAADATRTSHWLGANRWSYSFGADVIP |
| | | KVRDLSPEVLRSIKKPENVFRDALGFPGVLANAWRTSMICSVCGTDP |
| | | IGALEEAIAANQISFVTDNEGEGSLDLGDGRKVTLRVEVPTSSALTKR |
| | | EASRRKRRAPWEAKVGTVWTLTRKSHRDDLLTTIRRSLRRPSSTFQG |
| | | STTKQWEFHCPCCGQIQQADVNAASNLVRRYFVRASDNARARQH |
| | | WADDSKRLAFIASMGPDRSAREEKVS |
| 159 | Cas12 | MNRIYQGRVTKVEKLKNGKSPDDREELKDWQTALWRHHELFQDA |
| | | VSYYTLALAAMAEGLPDKHPINVLRKRMEEAWEEFPRKTVTPAKNL |
| | | RDSVRPWLGLSESASFGDALKKILPPAPENKEVRALAVALLAEKARTL |
| | | KPQKTSASYWGRFCDDLKKKPNWDYSEEELARKTGSGDWVAGLW |
| | | SEDALNKIDELAKSLKLSSLVKCVPDGQINPEGARNLVKEALDHLEGV |
| | | SNGTKKEKNDPGPAKKTNNWLRQHASDVRNFIHKNKNQFSSLPNG |
| | | RLITERARGGGININKTYAGVLFKAFPCPFTFDYVRAAVPEPKVKKVD |
| | | QEKKSEQSATWTELEKRILRIGDDPIELARKNNKPIFKAFTALEKWSD |
| | | QNSKSCWSDFDKCAFEEALKTLNQFNQKTEEREKRRSEAEAELKYM |
| | | MDENPEWKPKKETEGDDVREVPILKGDPRYEKLVKLFGDLDEEGSE |
| | | HATGKIYGPSRASLRGFGKLRNEWVDLFTKANDNPREQDLQKAVT |
| | | GFQREHKLDMGYTAFFLKLCERDYWDIWRDDTEVEVKKIREKRWV |
| | | KSVVYAAADTRELAEELERLQEPVRYTPAEPQFSRRLFMFSDIKGKQ |
| | | GAKHIREGLVEVSLAVKDQSGKYGTCRVRLHYSAPRLIRDHLSDGSS |
| | | SMWLQPMMAALGLSSDARGCFTRDSKGNVKEPAVALMSDFVGR |
| | | KRELRMLLNFPVDLDISKLEENIGKKARWEKQMNTAYEKNKLKQRF |
| | | HLIWPGMELKETQEPGQFWWDNPTIQKEGMYCLAIDLSQRRAAD |
| | | YALLHAGVNRDSKTFVELGQAGGQSWFTKLCAAGSLRLPGEDTEVI |
| | | REGKRQIELSGKKGRNATQSEYDQAIALAKQLLHNENSAELESAARD |
| | | WLGDNAKRFSFPEQNDKLIDLYYGALSRYKTWLRWSWRLTEQHKE |
| | | LWDKTLDEIRKVPYFASWGELAGNGTNEATVQQLQKLIADAAVDL |
| | | RNFLEKALLHIAYRALPLRENTWRWIENGKDGKGKPLHLLVSDGQS |
| | | PAEIPWLRGQRGLSIARIEQLENFRRAVLSLNRLLRHEIGTKPEFGSST |
| | | CGESLPDPCPDLTDKIVRLKEERVNQTAHLIIAQSLGVRLKGHSLFTEE |
| | | REKADMHGEHEVIPGRSPVDFVVLEDLSRYTTDKSRSRSENSRLMK |
| | | WCHRKINEKVKLLAEPFGIPVIEVFASYSSKFDARTGAPGFRAVEVTS |
| | | EDRPFWRKTIEKQSVAREVFDCLDNLVGKGLNGIHLVLPQNGGPLFI |
| | | AAVKEDQPLPAIRQADINAAVNIGLRAIAGPSCYHAHPKVRLIKGES |
| | | GTDKGKWLPRKGKEANKRENAQFGNVDLDLEVKFNRLDIDSDVLK |
| | | GDNTNLFHDPLNIACYGFATIQNLQHPFLAHASAVFSRQKGAVARL |
| | | QWEVCRAINSRRLEAWQKKAEKAAVKR |
| 160 | Cas12 | MSHELAEQPSPPKNKEPTCEESDAIRKNRRILLALSWLSVEDDCSAP |
| | | TGTFRVASGKDSEAERKNKVLTAFRSILTARRMRSQDVESWIADCA |
| | | ASLSAKIREDAVWINRSACFDQRALDLKVLSREYAKAAVMSFFGPLD |
| | | EYFKLPDEADDTKPAVGGDGPDFRTLARQWVSTNFGTGKKSDSEAI |
| | | AQNLRKLADANI.APFSGKPKAALIAHLSVELDGSTADIDGLCRAIGW |
| | | NTGRPSKGRVAIERLPDPPTETSIQTMQQKFREEAEAKASSKGLRQ.V |
| | | PEWMPAFQKSIERDCGMPFKLGEGRDHIGEFSVMLDHAARRVSIG |
| | | HSWIKRAEAERRRFEADAQRLNHIPAAAKDWLDQFVQFRSGSSGA |
| | | AAAGGEYRIRRRAIEGWDEIIKRWKRAACKSPEDRVAAAREVQADP |
| | | EIEKFGDIQLFEALAADDAECVWRGDGNGTPDPLKDYVAATDALDK |
| | | MRRFKVPAYRHPDPLAHPVFGDFGNSRGDIRFAVHEAAKATRGTK |
| | | RIAKDQKEWIRERHGLRMGLWDGQSVRTADLRWSSKRLVDDLALR |
| | | NHVTTRRTGPVSRADRLGRAAAGLGADEAACVAGLFELPDWNGRL |
| | | QAPRAQLDAIAACVAANGGKWDDKARKLRDRIEWLVSFSAKLECC |
| | | GPFMEYASQNGIQPNGKGEYYPHAERNKGRTGHAKLILSRLPGLRV |
| | | LAVDLGHRFAAACAVWEALSKIAFDAETKGREVVSGGRAADDLYC |
| | | HTRHLDCAGKARTTIYRRIGPDKLPDGSDHPAPWARLDRQFLIKLQ |
| | | GEERPARAAGPAETAAVQQIETDLGRARGQEDLPPRPVDSLMREA |
| | | VRTIRIALRRHGDAARIAYAFKPGAKRLKPGGGAQDHTPETHADAIL |
| | | EALLRWHELATGARWRDPWAETQWKDWVQPHISATLPELANDA |
| | | DRWERKRHRAALEQVLRPVAQMLIQRPTDALHQVWSKHWADED |
| | | LKWPSRLRWLRNWLLPRGPRARSGAARNVGGLSLLRIATLRELYQT |
| | | QKAYAMRPEPDDPRKRIAGRNDDRYDELGRSVLQVIERLREQRVK |
| | | QLASRIVEAALGVGRAKPTRGRQRPQSRVDVPCHAVIIESLRNYRPD |
| | | ELQTRRENRAIMNWSAGKVRKYLEEACQLHGLHLREVMPNYTSRE |
| | | DSRTGLPGVRCVDVPVDPKLGKPKAYWWNSVLSTARKKSIGDAAS |
| | | HDKQGDATSRFIVELAGCLDRLKADGKPLPKTVRVPRIGGDLFVAAP |
| | | PTSCTAPAHQPHPACDGARALQADLNAAANIGLRALLDPDFPAKW |
| | | WYVPCIDDQRGLALPRADKVLGSACFPGDPATFGSLLKTRTAAGPA |
| | | VDGQAAPDRKPRTGTHRPGSAKSRSLGDGKATTNYWSDRSARDLR |
| | | PADEGGHWQPTNVYWNWVRKRALLGLYSFNGLSPPSDDRP |
| 161 | Cas12 | MAIRSIKLKLKTNTGPEAQNLRKGIWRTHRLLNEGVAYYMNMLLLF |
| | | RQESTDKKTKQEIHEELIRHIRAQQQRNHADEKTQALPLEKALEALR |
| | | KLYELLVPSSVGQSGDSQIISRKFLSPLVDPNSEGGKGTSKAGAKPA |
| | | WQKKKEANDPTWKQDYEKWKKRREEDPTASVITTLEEYGIRPLFPL |
| | | YTNTVAEIAWLPLKSGQFVRTWDRDMFQQAIEGMLSWESWNRR |
| | | VQEEYAKLEGKMAQLNEQLEGGEEWIRLLEQYEEKREQELRENMT |
| | | AANDKFRITKRQMKGWKELYEVWSTFLPSASQEQYKEAIKRVQQR |
| | | LRGKFGDFHFFQYLSEEENRLIWKGNPQRIHYFVARNELTKKLEKAK |
| | | QSARRTLPDANKHPLWVRYDARGGNLQDYYLTAESDKPRSRRFVTF |
| | | SQLIWPSESGWLEKKDVQAELALSRQFYQQVTFLKNDKGKQEIEFK |
| | | DKGSGTTFSGHLGGAKLQLERSVLENKERKFEEGEIGKAYLNVAIDFK |
| | | PLQEVKNGRVQAPYGQVLQLIRLPNAFPKVRTYKSEELVEWIKASPQ |
| | | HLSGVESLASGFRVMSIDLGLRAAAATSIFSVEESSDKNATKLAYWIE |
| | | GTPLVAVHQRSYMLRLPGEQVEQHVWEKRDERGDQHKRVRFQIR |
| | | RLAEIIRLANKQYGDRWDELNRLDEAVSKEKSPLDQADRTFWEGIVS |
| | | DLTTALPLNDADWTEAVVQIHRKAELYVGKWQAWRKRFNADERK |
| | | GIAGLSMWSIEELDGLRKLLISWSRRTRNPQEVNRFEPEHTGHKRLL |
| | | THIQNVKKDRLKQVSHAIVMTALGYIYDEKKQKWCAKYPACQVILF |
| | | ENLSQYRSNLDRSAKENSTLMKWAHRSIPKYVHMQAEPYGIQIGD |
| | | VRAEYSSRYYAKTGTPGIRCKKLREHDVKGWRLDHLKKRLVNEQFLT |
| | | EAQVEQLKAGDIIPDDSGELFMTMTDGSGGKEIVFLQADINAAQNL |
| | | QKRFWQRNSELFKVSCRVIVRDEAEYLVPQAKKVQEKLGKGVFVKK |
| | | SDTAWKEVYVWDSQAKLKGKTTFTEESESPEQLEDLQEMIEEAEEA |
| | | KGTYRTLFRDPSGVFFPDFVWNTPKDFWGEVKRKLYGKLRERLLTK |
| | | VR |
| 162 | Cas12 | MNRIYQGRVTKVEVPDGKDEKGNIKWKKLENWSDILWQHHMLF |
| | | QDAVNYYTLALAAISGSAVGSDEKSIILREWAVQVQNIWEKAKKKA |
| | | TVFEGPQKRLTSILGLEQNASFDIAAKHILRTSEAKPEQRASALIRLLEE |
| | | IDKKNHNVVCGERLPFFCPRNIQSKRSPTSKAVSSVQEQKRQEEVRR |
| | | FHNMQPEEVVKNAVTLDISLFKSSPKIVFLEDPKKARAELLKQFDNA |
| | | CKKHKELVGIKKAFTESIDKHGSSLKVPAPGSKPSGLYPSAIVFKYFPV |
| | | DITKTVFLKATEKLAMGKDREVTNDPIADARVNDKPHFDYFTNIALI |
| | | REKEKNRAAWFEFDLAAFIEAIMSPHRFYQDTQKRKEAARKLEEKIK |
| | | AIEGKGGQFKESDSEDDDVDSLPGFEGDTRIDLLRKLVTDTLGWLGE |
| | | SETPDNNEGKKTEYSISERTLRIFPDIQKQWSELAEKGETTEGKLLEVL |
| | | KHEQTEHQSDFGSATLYQHLAKPEFHPIWLKSGTEEWHAENPLKA |
| | | WLNYKELQYELTDKKRPIHFTPAHPVYSPRYFDFPKKSETEEKEVSKN |
| | | THSLTTSLASEHIKNSLQFTAGLIRKTNVGKKAIKARFSYSAPRLRRDC |
| | | LRSENNENLYKAPWLQPMMRALGIDEEKADRQNFANTRITLMAK |
| | | GLDDIQLGFPVEANSQELQKEVSNGISWKGQFNWGGIASLSALRW |
| | | PHEKKPKNPPEQPWWGIDSFSCLAVDLGQRYAGAFARLDVSTIEKK |
| | | GKSRFIGEACDKKWYAKVSRMGLLRLPGEDVKVWRDASKIDKENG |
| | | FAFRKELFGEKGRSATPLEAEETAELIKLFGANEKDVMPDNWSKELS |
| | | FPEQNDKLLIVARRAQAAVSRLHRWAWFFDEAKRSDDAIREILESD |
| | | DTDLKQKVNKNEIEKVKETIISLLKVKQELLPTLLTRLANRVLPLRGRS |
| | | WEWKKHHQKNDGFILDQTGKAMPNVLIRGQRGLSMDRIEQITELR |
| | | KRFQALNQSLRRQIGKKAPAKRDDSIPDCCPDLLEKLDHMKEQRVN |
| | | QTAHMILAEALGLKLAEPPKDKKELNETCDMHGAYAKVDNPVSFIVI |
| | | EDLSRYRSSQGRSPRENSRLMKWCHRAVRDKLKEMCEVFFPLCERR |
| | | KAGSAWVSLPPLLETPAAYSSRFCSRSGVAGFRAVEVIPGFELKYPW |
| | | SWLKDKKDKAGNLAKEALNIRTVSEQLKAFNQDKPEKPRTLLVPIAG |
| | | GPIFVPISEVGLSSFGLKPQVVQADINAAINLGLRAISDPRIWEIHPRL |
| | | RTEKRDGRLFAREKRKYGEEKVEVQPSKNEKAKKVKDDRKPNYFAD |
| | | FSGKVDWGFGNIKNESGLTLVSGKALWWTINQLQWERCFDINKRH |
| | | IEDWSNKQKQ |
| 163 | Cas12 | MGEENNFSQFTGLYELSKTLRFELKPVWETEKLLIENQVFPKDKIVYE |
| | | SYKKIRPYLDKLHLQFIEESLSSVKLDFNEIEKKFLEWDKEKDKTTKNKL |
| | | KEEIFWKNKKWWLNSNLRKDMVSYFNLNWKKWINNFENKTIVNE |
| | | KWKEEKIKIKWEWYKILLSDSNLNILSDFFTQEKEWDNVLIEKYIENP |
| | | NFKENDWEKKFILEKQNLFKSFKWFTTYFTNFNHSRENFYKDDWKS |
| | | WRIATRIIDENLIFFLKNKKAFDEKYKNNSEITVKFNEKLLNFWEKLED |
| | | FFSLDFYNRCYTWKQIEYYNQLIWELNSVVNHEKQAKFSEYTQNKK |
| | | VSENNKFNKYDFPIFKELYKQILSEKATEQKFIEINDFKELKQNLQELIE |
| | | KNKQKNKFAINLTKSLIEKIDEYDFEKIYISKLSLNTISSKFFWSNKWFFI |
| | | QENLEKNIWKKNKNWKIDLPDFIKLSDIKTALENFNKILFEDKENKEN |
| | | IFKEEFDNITENNIFIKFMKIYENEFINLIEWKKNNKWDYEIIWYNKSL |
| | | LDLEKNILNISDFSDKKEEKEKQIEYIKKYLDSSLNIYRVMKYFALEKGK |
| | | ESVEWEFETDDIFYNEFKKFYIDNEIISYYNEFRNYLTKKPYSSNKLKLN |
| | | FENWTLIDGWDKNKEPDNYWTILRKNWKYFLALQIKWKNNIFYKK |
| | | KWSWVIDIEEAYKIDLWDEFYEKMDYKFLPDPKKMLPKVIFAKSNV |
| | | KLFNPSKEILQIKENETFKTWDKFKVNDFYKIVDFYKENIIKYPDWKIF |
| | | NFKFSDTKTYNNLSDFYKEIELWSYDLNFRKVSKKYILKSIEEKNIYLFEI |
| | | YNKDFADWKTWSENLHTMYFKWLFEENNLNNIVLKLNWQAEIFR |
| | | REASLKEKEVNRAKENKEKSHNIIENARYTKDKLFFHCPIKLNFAKHN |
| | | EKINQEILKYISDNKEINIIWIDRWEKHLAYYSVINRDWNIIKDKNWN |
| | | LVKWSLNIVWNNQNYHDKLETREKERQDARWSWKTIWNIKDLKQ |
| | | GYISQVVHKLAELVIEHNAIIVFEDLNSWFKRWRQKIERQVYQKLEK |
| | | ALIEKLNYLTFKDKNFWENWHYLKAYQLTAPFETFEKVWKQTWVIF |
| | | YTDPSYTSSTCPACWFRKDLYLKYSNLKNALWDIEKIDSIIFDWKRFIF |
| | | EYNNKKVFTDRDRKRNISSEESWIKWWKTIDNNITEFLEILFKKSNID |
| | | YTSWNNLILDIQEINEKELYKWVFDNFNAILNIRNSTIKDNSRTWDFI |
| | | CCPACDFDSRKENKIWIENWDDNWAFNIARKWIIILNKIDSYLNEK |
| | | WSLDKILWWDMIVKQIDWDDFTHKK |
| 164 | Cas12 | MAQASSTPAVSPRPRPRYREERTLVRKLLPRPGQSKQEFRENVKKLR |
| | | KAFLQFNADVSGVCQWAIQFRPRYGKPAEPTETFWKFFLEPETSLP |
| | | PNDSRSPEFRRLQAFEAAAGINGAAALDDPAFTNELRDSILAVASRP |
| | | KTKEAQRLFSRLKDYQPAHRMILAKVAAEWIESRYRRAHQNWERN |
| | | YEEWKKEKQEWEQNHPELTPEIREAFNQIFQQLEVKEKRVRICPAA |
| | | RLLQNKDNCQYAGKNKHSVLCNQFNEFKKNHLQGKAIKFFYKDAEK |
| | | YLRCGLQSLKPNVQGPFREDWNKYLRYMNLKEETLRGKNGGRLPH |
| | | CKNLGQECEFNPHTALCKQYQQQLSSRPDLVQHDELYRKWRREYW |
| | | REPRKPVFRYPSVKRHSIAKIFGENYFQADFKNSVVGLRLDSMPAGQ |
| | | YLEFAFAPWPRNYRPQPGETEISSVHLHFVGTRPRIGFRFRVPHKRS |
| | | RFDCTQEELDELRSRTFPRKAQDQKFLEAARKRLLETFPGNAEQELR |
| | | LLAVDLGTDSARAAFFIGKTFQQAFPLKIVKIEKLYEQWPNQKQAGD |
| | | RRDASSKQPRPGLSRDHVGRHLQKMRAQASEIAQKRQELTGTPAP |
| | | EIIIDQAAKKATLQPFDLRGLTVHTARMIRDWARLNARQIIQLAEE |
| | | NQVDLIVLESLRGFRPPGYENLDQEKKRRVAFFAHGRIRRKVTEKAV |
| | | ERGMRVVTVPYLASSKVCAECRKKQKDNKQWEKNKKRGLFKCEGC |
| | | GSQAQVDENAARVLGRVFWGEIELPTAIP |
| 165 | Cas12 | MGEENNFSQFTGLYELSKTLRFELKPVWETEKLLIENQVFPKDKIVYE |
| | | SYKKIRPYLDKLHLQFIEESLSSVKLDFNEIEKKFLEWDKEKDKTTKNKL |
| | | KEEIFWKNKKWWLNSNLRKDMVSYFNLNWKKWINNFENKTIVNE |
| | | KWKEEKIKIKWEWYKILLSDSNLNILSDFFTQEKEWDNVLIEKYIENP |
| | | NFKENDWEKKFILEKQNLFKSFKWFTTYFTNFNHSRENFYKDDWKS |
| | | WRIATRIIDENLIFFLKNKKAFDEKYKNNSEITVKFNEKLLNFWEKLED |
| | | FFSLDFYNRCYTWKQJEYYNQLIWELNSVVNHEKQAKFSEYTQNKK |
| | | VSENNKFNKYDFPIFKELYKQILSEKATEQKFIEINDFKELKQNLQELIE |
| | | KNKQKNKFAINLTKSLIEKIDEYDFEKIYISKLSLNTISSKFFWSNKWFFI |
| | | QENLEKNIWKKNKNWKIDLPDFIKLSDIKTALENFNKILFEDKENKEN |
| | | IFKEEFDNITENNIFIKFMKIYENEFINLIEWKKNNKWDYEIIWYNKSL |
| | | LDLEKNILNISDFSDKKEEKEKQIEYIKKYLDSSLNIYRVMKYFALEKGK |
| | | ESVEWEFETDDIFYNEFKKFYIDNEIISYYNEFRNYLTKKPYSSNKLKLN |
| | | FENWTLIDGWDKNKEPDNYWTILRKNWKYFLALQIKWKNNIFYKK |
| | | KWSWVIDIEEAYKIDLWDEFYEKMDYKFLPDPKKMLPKVIFAKSNV |
| | | KLFNPSKEILQIKENETFKTWDKFKVNDFYKIVDFYKENIIKYPDWKIF |
| | | NFKFSDTKTYNNLSDFYKEIELWSYDLNFRKVSKKYILKSIEEKNIYLFEI |
| | | YNKDFADWKTWSENLHTMYFKWLFEENNLNNIVLKLNWQAEIFR |
| | | REASLKEKEVNRAKENKEKSHNIIENARYTKDKLFFHCPIKLNFAKHN |
| | | EKINQEILKYISDNKEINIIWIDRWEKHLAYYSVINRDWNIIKDKNWN |
| | | LVKWSLNIVWNNQNYHDKLETREKERQDARWSWKTIWNIKDLKQ |
| | | GYISQVVHKLAELVIEHNAIIVFEDLNSWFKRWRQKIERQVYQKLEK |
| | | ALIEKLNYLTFKDKNFWENWHYLKAYQLTAPFETFEKVWKQTWVIF |
| | | YTDPSYTSSTCPACWFRKDLYLKYSNLKNALWDIEKIDSIIFDWKRFIF |
| | | EYNNKKVFTDRDRKRNISSEESWIKWWKTIDNNITEFLEILFKKSNID |
| | | YTSWNNLILDIQEINEKELYKWVFDNFNAILNIRNSTIKDNSRTWDFI |
| | | CCPACDFDSRKENKIWIENWDDNWAFNIARKWIIILNKIDSYLNEK |
| | | WSLDKILWWDMIVKQIDWDDFTHKK |
| 166 | Cas12 | MATAINYPTTQRAYTLRLRGIDPQDQSWRDALWATHEAVNRGAK |
| | | VFGEWLLTLRGGLDHQLADAPVKVRGGTTRLPSDEERRDRRVLLAL |
| | | SWLSVEDAHGAPPDASLIVAKGTDSADCRARKLADALIAILQARSVA |
| | | ASEIGDPSKPPEDQPGTWLGDCMGSLSAAIRDDAVWVNRSKAFDA |
| | | ATQSCPSLTRDEIWDFLEPFFASPDAYLKPERAESDEGDSTSAATEDK |
| | | AKDLVQKAGGWLSKRMGAGGGANFQDLARAYQAIAQWASSAQP |
| | | GQSAQQAVGSLAGYLSQHGFSPTANDATGVLAVISGPGYKSATRN |
| | | HITAIATSPEITPQDLSKLQELATKDKAGCSSKIGGKGPRPYATMILQ |
| | | QVEAACGFTYLQSDGPARHREFSVMLDHAARRVNVAHSWIKNAE |
| | | AERRQFESDARRIKKVPQDALNWLRGYCEERGGASGSLEGYRIRRR |
| | | AIDGWDQVVIRWSRSDCQSADDRIAAARQLQDDPEIDKFGDIQLFE |
| | | ALAAEEALCVWKPDGNPTAQPLKDFVAATEADAKKKRFKVPAYRH |
| | | PDPLRHPVFTDFGNSRWGIEYSAHRAPAKCDELGQQVDRLTQAVA |
| | | EAQRNLDGATAAQRASRESKLAEAQSKLVAAQTEFAAINDPHRVEL |
| | | KLWNGQAVAAIPMRWSSKRLIADLSLRRATQPSSDQRIGVTRADRL |
| | | GRAAGNADDGRPVTITGLFQQDHWNGRLQAPRAQLDAIAKHVDK |
| | | HGWDAKARRQIARIRWVVSFSAELSQQGPWFEFCHRFGEDAPAR |
| | | PFVSRHGEYAVKHRDNDQRKGHAKLILSRLPGLRVLAVDLGHRYAA |
| | | ACAVWEAISSDQMRQACAAANAPAPHPLAMYIHLKSTTAKGKPTT |
| | | TIYRRIGPDKLPDGTPHPAPWARLDRQFLIKLPGEDRPARAASPDEIK |
| | | AVEDFEDSVGRVRTAVDPPRKRGVDLLMHDAVRTARLALARHGRR |
| | | ARIAFQLISQVRILPGGRPQTLDDAGRRDLLNDTLADWYALATDSR |
| | | WTDAAARQLWNERLAALNGGFTIDPPADASQPEAERTRAQRRQA |
| | | EQELRHRLASLVEALFCNPTLCQQLHQAWTDRWNADDQQWRSRL |
| | | KWLSRWLLPRGGSRRDGSRRHVGGLSLTRISTLIDFRRKVQVGYFTR |
| | | LRPDGSRAEIGPQFGQSTLDAIQRLKDQRIKQLTSRIVEAALGIGVEQ |
| | | DRIWDAAKRKWRTVKRPREPRYHVDDQGVQQRDPRFQACHAVVI |
| | | EDLSHYRPEETRTRRENRATMDWKSAETRKRLADHCQLYGLHLRD |
| | | VNPQYTSRQDSRTGAPGCRCVDVSVADFLTKPAWRKQVAQARGK |
| | | VASNRGDARDRLLVELDHQLTTANSLRGEMDSLRIPVNGGEVFVSA |
| | | DPRSPLAAGIQADLNAAANIGLRALMDPDFLGTWWYVPCDPSTKK |
| | | PHIEKVKGSILATVGALQATSEEAAAPPRRGRGGTRSAAPREVINLW |
| | | RDPSAVRIQDATAGEVWDVTPVYWSIVKDRVVDVLRQRNTKSGD |
| 167 | Cas12 | MAVRSVKLKLLVPRDGSAESVRKRKALWATHQFVNDAAAAYAELLL |
| | | EMRQEDVCRGTDDHGKDVIEPAAHWQAKLRARLAAKQLPPVAVA |
| | | EALPLLKAFYGSRLIKSFVANDKGVAGTGNATDLNTWLSGLVDPASV |
| | | AGEKTELRKQLLAELPLCEAADADFEGAARKMLAKSDAREALLEGP |
| | | GTGVGWPAAYNANPTDSVWLDMLHKAAAKARLELADTTVSELKK |
| | | LGVFPLLQAASSNRVFGSGVLNPFERMAAAQAAAALLPWETKRHE |
| | | MRKRRDKFADQLNQWDTEFGASHATALAAIRAFEAEESERARRESL |
| | | GNEGTGYRIGGRELRDAWTLLRDWLKGHSTATAAAREDKVRELQA |
| | | KQGRSFGSHRLLSWLAKPAQQWLADHSAGDVVTRIAVRNARQRK |
| | | LDTARTLPIWTGADAVKHPRFANFDPPNNTNQPGFDLRAGTQKGR |
| | | LTLRLSLLTERADGLLLAQDHDFQLVPSRQMAEIVLHKDGKERALSW |
| | | QSQDGIGRQVGDVGGSALLFSRDHAECLLERKQITRLERGAWPAAL |
| | | PVWFKLSLDIGAEHKALLKQRFKWGVWLNSALVTRNAKDAKGVPP |
| | | PVGTRVLAVDLGLRSAATVSVWQVVDAATPVVAGKWRVPLSDTLS |
| | | AVHERSAMLALPGEHVDAGVLAARRAANEKLAGLLAATSHLSTVFK |
| | | LGRAEQGDRRRELLERLGEGDDRRARAAVATTAAERDGLRAVLGA |
| | | TQDAWAGAVAAVWRRLETDLAGAIAAYRKQQREDVQLRREARHG |
| | | PGASQLPKQAAAERLLGGKSAWQIEYKERVRKLLTRWIMRQRPGD |
| | | TAVRRLARKDLGKYCGGLLDHLTALKEDRAKTTADLIVQAARGRVR |
| | | AHKDAHGRQQDRELWLAKYAPCDLIVMEDLGRYRFATDRPPSENR |
| | | QLMQWTHREVFRLVQMQAEVEGIQVLETGAEFSSKFDARTWAPG |
| | | VRCEPITKLWVERYRNGEMPWLADKADEWRREGIELAQLVPGQLL |
| | | PTGSGEQFVAVSATGGLRVRHADLNAAQCIALRALTGHGTAFRLTA |
| | | RRLGDVFVSAKGLGKRPQGALWREFGSALPPAVVVLRPAGEVRYAL |
| | | RPFASARDAAAALGLQLGALRNVDATDAESDAEDGDLAELLAGAD |
| | | PDRATFFRDPSGDVHGGAWVQAKVFWAEVRRHVRLGLQAQGLLP |
| | | AAARSSEPRQMQLPLAGALPGDDIPL |
| 168 | Cas12 | MAAFQRSYTMNLKPATSEQDKFILWNRLFLTHWSVNEGAKIFGELF |
| | | LNLRGGLSPELGIFDLNKDKDDRKKKALVMGRRRLLALGWLSVEDN |
| | | LSAGDHPFRIREIPVGRNMEIKQATTLLTEILKNKGVKDEAVIKEWID |
| | | DCTPSLIANIREDAVWINRAKSFYSMNPCPTKDEVWKILSYVLNTSFL |
| | | DLSLNDSSERDNTKNKKGTKENEKDVSNKSKELYGWLFTKNPNKM |
| | | REAGENKDKFINNFRENFNTFTDYANLKVEIELWRKNNISNTLLITQK |
| | | AKYPPEVKEANHPSKFSVGYRKLLVHLELWPSSKDENGDIPKGIEGK |
| | | DKSHNQILLDYLLEVCNEGNKTTKKVIVPAWADGIKTELESKASIKVG |
| | | DSTSSVLQRLM1KMAARRISQTLSWIKINEQVRHDAYQKKNKAFKLL |
| | | CEIDKNGEACKWLENYELFRRDDSGGEEYHISARAISCWKQILEEW |
| | | QKNDSSKALREKVKVVQAAEDKFGDARLFEDLADDNARSVWLLPD |
| | | GNKTPDILNWWCEYRTADIDESRFKIPCYCHPHPFKHPVYVEYGKS |
| | | NPQVIFSLKHDKARKNRIDNGWNPKNPRILALLLLDIVRQKSTLAPF |
| | | VWESKRLWKDLGGDATVTYKIPRSDRMGLSSIGNIDYARPEVPFLKE |
| | | KWNARLQSDRRTLEKLEKYWNPESMKWIDDGKFLIQSKWFITFGP |
| | | DMETAEGPWKLYLKENINDNNYLGNRSKENQKRGYRAKKLLSGYP |
| | | AGMRILSVDLGHRYAASCAIWETITKKQITEELAYQPDKNSVFEHSC |
| | | KTIDKKIKNTVYRRIGDDSIDAPWAKLEKQFTIKLQGEDKSCYLLRSDE |
| | | KELFRSILSKLSCLNNDTGHNILEMIENLLRIVKAKIYRQGILARISYSM |
| | | TAQYKPGKGGQKSPLSDEDKIHYLSENLAAWSALMGNQEWNEDGI |
| | | SDWYKKYISHLVSGPKPKEGNRKSDRDKIIEYFLPAARKLYDDNETRI |
| | | NIHDLFKELWDENNKQLSAVLKEIKKIILPKGIRYFDKNNDSSSRWKN |
| | | NQSKLKQITHRGGLSLRRIVAIEGYYKLAKAYKNHPEPDNLTKNIPLP |
| | | GDNSSAGFNQRIRDTLERMKEQRVKQIASRIVESALGLGIEGYKKRP |
| | | LTPESKPCQAIVIEDLSHYRPDELQTRRENRRLMQWSSSKVKKYLSE |
| | | ACEMHDVLLVEISPEYTSRQDSRTGVAGLRCIDINIREFLKDSSSWQ |
| | | NKIKTIQMKPTNKKSNLDQYLIELNESLGERYKDKVIPSDKFVRIPRKG |
| | | GDIFVSSSKESPVSKGIQADLNAAANIGLKALLDPDWAGAWWYILIE |
| | | AKSNHVIPYGKKYKGAECLRDFKFSGLENQVMKNNMNLWRDLQS |
| | | QFSSEDKWMSYKEYNELTEKRVINILRERAGLELIEE |
| '169 | Cas12 | MPTRTINLKMVLGRKDDTAELRRALWTTHEHVNLAVAEVERVLLRC |
| | | RGRSYWTLDRRGDPVHVPESQVAEDALAMAREAQRRNGWPVVG |
| | | EDEEILLALRYLYEQIVPSCLLDDLGKPLKGDAQKIGTNYAGPLFDSDT |
| | | CRRDEGKDVACCGPFHEVAGKYLGALPEWATPISKQEFDGKDASHL |
| | | RFKATGGDDAFFRVSIEKANAWYEDPANQDALKNKAYNKDDWKK |
| | | EKDKGISSWAVKYIQKQLQLGQDPRTEVRRKLWLELGLLPLFIPVFD |
| | | KTMVGNLWNRLAVRLALAHLLSWESWNHRAVQDQALARAKRDE |
| | | LAALFLGMEDGFAGLREYELRRNESIKQHAFEPVDRPYVVSGRALRS |
| | | WTRVREEWLRHGDTQESRKNICNRLQDRLRGKFGDPDVFHWLAE |
| | | DGQEALWKERDCVTSFSLLNDADGLLEKRKGYALMTFADARLHPR |
| | | WAMYEAPGGSNLRTYQIRKTENGLWADVVLLSPRNESAAVEEKTF |
| | | NVRLAPSGQLSNVSFDQIQKGSKMVGRCRYQSANQQFEGLLGGAE |
| | | ILFDRKRIANEQHGATDLASKPGHVWFKLTLDVRPQAPQGWLDGK |
| | | GRPALPPEAKHFKTALSNKSKFADQVRPGLRVLSVDLGVRSFAACSV |
| | | FELVRGGPDQGTYFPAADGRTVDDPEKLWAKHERSFKITLPGENPS |
| | | RKEEIARRAAMEELRSLNGDIRRLKAILRLSVLQEDDPRTEHLRLFME |
| | | AIVDDPAKSALNAELFKGFGDDRFRSTPDLWKQHCHFFHDKAEKVV |
| | | AERFSRWRTETRPKSSSWQDWRERRGYAGGKSYWAVTYLEAVRG |
| | | LILRWNMRGRTYGEVNRQDKKQFGTVASALLHHINQLKEDRIKTGA |
| | | DMIIQAARGFVPRKNGAGWVQVHEPCRLILFEDLARYRFRTDRSRR |
| | | ENSRLMRWSHREIVNEVGMQGELYGLHVDTTEAGFSSRYLASSGA |
| | | PGVRCRHLVEEDFHDGLPGMHLVGELDWLLPKDKDRTANEARRLL |
| | | GGMVRPGMLVPWDGGELFATLNAASQLHVIHADINAAQNLQRRF |
| | | WGRCGEAIRIVCNQLSVDGSTRYEMAKAPKARLLGALQQLKNGDA |
| | | PFHLTSIPNSQKPENSYVMTPTNAGKKYRAGPGEKSSGEEDELALDI |
| | | VEQAEELAQGRKTFFRDPSGVFFAPDRWLPSEIYWSRIRRRIWQVT |
| | | LERNSSGRQERAEMDEMPY |
| 170 | Cas12 | MVTRALNLKLVVPRRPGELTKAEALWSTHDIVNRATSYYESQLLLCR |
| | | QQDYQTRELTVSAGDQAPDLDALIANARDRNRYRGLEKPQVVREK |
| | | LRNLYEAIVPPAIGKTGTAQAVGAFVSPLLDADSRGFTEIFDKIEALPN |
| | | WVDGVRAEEPDALEAAADWLKSPQGKERLRPTGAPPTWIKLAKKK |
| | | DAGWAAAFVADIDKKLKEVEGTPTLMQELRALGVMPLFPSFFASRI |
| | | AGHKGAVSTWDRLALRLAVAHLLSWESWVELAAKEHAARVAKLEK |
| | | FRDDNILGEIADAVEALRLYEKERTEELQQKAQLDAEEVRTTSRTIRG |
| | | WVDLREKWLKTDASPDALISLVAAEQKRKSGKFGDPQLFRWLAKPE |
| | | NHFVWNKPDFDPPSLFASLRMIEGLVERSKETAWMTLPDARLHPR |
| | | SSQWEPHGGGNLKTFRLEQGEGGSLSVTLPLLRKSGDDSYVEEEHA |
| | | FSLAGSKQIPNASLDVRRNKYCLSYRTPTGEEAEAVVGSADLLLDWY |
| | | FLQQRSEHRPEEGDIGPAFLKLALDITPIDPVWGEREKTPAIHHFKTA |
| | | SGKNTRHADGVAPGFRMLAVDLGIRTLATCSVFELKATAPAGRLSFP |
| | | IAHLDLHAVHERSFTLTLDGEDPDRDAERWRENKSAELRRLRMGLT |
| | | RYRNIRNMREDAPDEREVLLEDLQEKVQEHGWAFEEPLLRELAKHK |
| | | DTPEPIWEAELTKALAQFRSDFGVIVGEWRRSNRARSTDSHAGKSM |
| | | WAIDHLTNSRRFLMSWSLLSKPGQIRRLDRDKQGVFAKHLLDHLEG |
| | | LKADRLKTGSDLIVQAARGFRRDKRGNWHKAYKPCHGILFEDLSRY |
| | | RMRTDRPRRENSQLMKWAHRAVPKEVGMQAEVYGIRVEDTGAA |
| | | FSSRFHAASHTPGIRMHPICQKDLENEWLLDEIEKQNSGVKRRELKL |
| | | GQLVQLNGGELFACVTASGVKTLHADINAAQNLQRRFFTRHGDAF |
| | | RIVARKVLVDEEEVWVPRSLGKRLLGALGSHGKLVPTGHESGSCRFE |
| | | EITTRAWSKLSGEKLSDDRVGNEEDQIIASIEEEALERTGEVVVFFRD |
| | | PSGQVLPRDLWYPSKTFWSIVKSTTLSKLKAAP |
| 171 | Cas12 | MEDKQFLERYKEFIGLNSLSKTLRNSLIPVGSTLKHIQEYGILEEDSLRA |
| | | QKREELKGIMDDYYRNYIEMHLRDVHDIDWNELFEALTEVKKNQT |
| | | DDAKKCLEKIQEKKRKEIYQYLSDDAVFSEMFKEKMISGILPDFIRCN |
| | | EEYSEEEKEEKLKTVALFHRFTSSFNDFFLNRKNVFTKEAIATAIGYRV |
| | | VHENAEIFLENMVAFQNIQKSAESQISIIERKNEHYFMEWKLSHIFTA |
| | | DYYMMLMTQKAIEHYNEMCGVVNQHMKEYCQKEKKNWNLYRM |
| | | KRLHKQILSNASTSFKIPEKYENDAEVYESVNSFLQNVMEKTVMERI |
| | | AVLKNNTDNFDLSKIYITAPYYEKISNYLCGSWNTIADCLTHYYEQQI |
| | | AGKGARKDQKVKAAVKADKWKSLSEIEQLLKEYARAEEVKRKPEEYI |
| | | AEIENIVSLKEVHLLEYHPEVNLIENEKYATEIKDVLDNYMELFHWMK |
| | | WFYIEEAVEKEVNFYGELDDLYEEIRDIVPLYNKVRNYVTQKPYSDTK |
| | | IKLNFGTPTLANGWSKSKEYDYNAILLQKDGKYYMGIFNPVQKPEKE |
| | | IIEGHSHPLEGNEYKKMVYYYLPSANKMLPKVLLSKKGMEIYQPSEYI |
| | | INGYKERRHIKSEEKFDLQFCHDLIDYFKSGIERNPDWKVFGFHFSDT |
| | | DTYQDISGFYREVEDQGYKIDWTYIKEADIDRLNEEGKLYLFQIYNKD |
| | | FSEKSTGRENLHTMYLKNLFSEENIREQVLKLNGEAEIFFRKSSVKKPII |
| | | HKKGTMLVNRTYMEEMHGESVKKNIPEKEYQEIYNYMNHRWKGE |
| | | LSAEAKEYLKKAVCHETKKDIVKDYRYSVDKFFIHLPITINYRASGKEA |
| | | LNSVAQRYIAHQNDMHVIGIDRGERNLIYVSVINMQGEIIEQKSFNV |
| | | VNKYNYKEKLKEREQNRDEARKNWKEIGQIKDLKEGYLSGVIHEIAK |
| | | MMIKYHAIVAMEDLNYGFKRGRFKVERQVYQKFENMLIQKLNYLV |
| | | FKDRSADEDGGVLRGYQLAYIPDSVKKLGRQCGMIFYVPAAFTSKID |
| | | PATGFVDIFNHKAYTTDQAKREFILSFDEICYDVERQLFRFTFDYANF |
| | | ATHNVTLARNNWTIYTNGTRTQKEFVNRRVRDKKEVFDPTEKMLK |
| | | LLELEGVEYQSGANLLPKLEKISDPHLFHELQRIVRFTVQLRNSKNEE |
| | | NDVDYDHVISPVLNEEGKFFDSSKYENKEEKKESLLPVDADANGAYC |
| | | IALKGLYIMQAIQKNWSEEKALSPDVLRLNNNDWFDYIQNKRYR |
| 172 | Cas12 | MAQASSTPAVSPRPRPRYREERTLVRKLLPRPGQSKQEFRENVKKLR |
| | | KAFLQFNADVSGVCQWAIQFRPRYGKPAEPTETFWKFFLEPETSLP |
| | | PNDSRSPEFRRLQAFEAAAGINGAAALDDPAFTNELRDSILAVASRP |
| | | KTKEAQRLFSRLKDYQPAHRMILAKVAAEWIESRYRRAHQNWERN |
| | | YEEWKKEKQEWEQNHPELTPEIREAFNQIFQQLEVKEKRVRICPAA |
| | | RLLQNKDNCQYAGKNKHSVLCNQFNEFKKNHLQGKAIKFFYKDAEK |
| | | YLRCGLQSLKPNVQGPFREDWNKYLRYMNLKEETLRGKNGGRLPH |
| | | CKNLGQECEFNPHTALCKQYQQQLSSRPDLVQHDELYRKWRREYW |
| | | REPRKPVFRYPSVKRHSIAKIFGENYFQADFKNSVVGLRLDSMPAGQ |
| | | YLEFAFAPWPRNYRPQPGETEISSVHLHFVGTRPRIGFRFRVPHKRS |
| | | RFDCTQEELDELRSRTFPRKAQDQKFLEAARKRLLETFPGNAEQELR |
| | | LLAVDLGTDSARAAFFIGKTFQQAFPLKIVKIEKLYEQWPNQKQAGD |
| | | RRDASSKQPRPGLSRDHVGRHLQKMRAQASEIAQKRQELTGTPAP |
| | | ElIIDQAAKKATLQPFDLRGLTVHTARMIRDWARLNARQIIQLAEE |
| | | NQVDLIVLESLRGFRPPGYENLDQEKKRRVAFFAHGRIRRKVTEKAV |
| | | ERGMRVVTVPYLASSKVCAECRKKQKDNKQWEKNKKRGLFKCEGC |
| | | GSQAQVDENAARVLGRVFWGEIELPTAIP |
| 173 | Cas12 | MAVKSIKVKLRLSECPDILAGMWQLHRATNAGVRYYTEWVSLMR |
| | | QEILYSRGPDGGQQCYMTAEDCQRELLRRLRNRQLHNGRQDQPG |
| | | TDADLLAISRRLYEILVLQSIGKRGDAQQIASSFLSPLVDPNSKGGRGE |
| | | AKSGRKPAWQKMRDQGDPRWVAAREKYEQRKAVDPSKEILNSLD |
| | | ALGLRPLFAVFTETYRSGVDWKPLGKSQGVRTWDRDMFQQALERL |
| | | MSWESWNRRVGEEYARLFQQKMKFEQEHFAEQSHLVKLARALEA |
| | | DMRAASQGFEAKRGTAHQITRRALRGADRVFEIWKSIPEEALFSQY |
| | | DEVIRQVQAEKRRDFGSHDLFAKLAEPKYQPLWRADETFLTRYALY |
| | | NGVLRDLEKARQFATFTLPDACVNPIWTRFESSQGSNLHKYEFLFDH |
| | | LGPGRHAVRFQRLLVVESEGAKERDSVVVPVAPSGQLDKLVLREEE |
| | | KSSVALHLHDTARPDGFMAEWAGAKLQYERSTLARKARRDKQGM |
| | | RSWRRQPSMLMSAAQMLEDAKQAGDVYLNISVRVKSPSEVRGQR |
| | | RPPYAALFRIDDKQRRVTVNYNKLSAYLEEHPDKQIPGAPGLLSGLR |
| | | VMSVDLGLRTSASISVFRVAKKEEVEALGDGRPPHYYPIHGTDDLVA |
| | | VHERSHLIQMPGETETKQLRKLREERQAVLRPLFAQLALLRLLVRCG |
| | | AADERIRTRSWQRLTKQGREFTKRLTPSWREALELELTRLEAYCGRV |
| | | PDDEWSRIVDRTVIALWRRMGKQVRDWRKQVKSGAKVKVKGYQL |
| | | DVVGGNSLAQIDYLEQQYKFLRRWSFFARASGLVVRADRESHFAVA |
| | | LRQHIENAKRDRLKKLADRILMEALGYVYEASGPREGQWTAQHPPC |
| | | QLIILEELSAYRFSDDRPPSENSKLMAWGHRGILEELVNQAQVHDVL |
| | | VGTVYAAFSSRFDARTGAPGVRCRRVPARFVGATVDDSLPLWLTEF |
| | | LDKHRLDKNLLRPDDVIPTGEGEFLVSPCGEEAARVRQVHADINAA |
| | | QNLQRRLWQNFDITELRLRCDVKMGGEGTVLVPRVNNARAKQLF |
| | | GKKVLVSQDGVTFFERSQTGGKPHSEKQTDLTDKELELIAEADEARA |
| | | KSVVLFRDPSGHIGKGHWIRQREFWSLVKQRIESHTAERIRVRGVGS |
| | | SLD |
| 174 | Cas12 | MSNPNIPNISPNITLTRDDVVNLLMSSIAMEELGLAHIINAEGEKIQF |
| | | ALGTLQGASGPPATLQQVLEVNQSTQAMLDTIFRQEMMLDSKLKT |
| | | ATNIPTLRGPTGPVGPTGAPGGVISINGQTGVVTLDASNGVMPFM |
| | | REQSTSSLDDYKDPGIYXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXGATGATGATGATGPQEPRALREPRAPQARREPRGLLEPQVL |
| | | RGPQEPRALREPRAPRALREPRALRALRALQELRAPRALRGLQEPRV |
| | | LQGPRERQVRPEPRVLQGPRERQVRQGLQGLLEPRAKRERQVRPE |
| | | PREPQERQAPLVQQALLEPQALLGQAL |
| 175 | Cas12 | MIKKSNFWQFTGLYELSKTLRFELKPVWETLNILEKDWVSQKDKEVE |
| | | ENYNKIKVFFDSLHREFVKQSLENWYLELLENFYNSYIELNKNIENKK |
| | | NKSLQKLFEKSSKELKKELVSFFEWKWNDWKQKYSFLKKWWIDVL |
| | | NEKEVLDLMWEFYPKEKELFKKFDKFFTYFSNFKESRKNFYADDWR |
| | | AWAIATRVIDENLITFIKNIEDFKKFKNNFSDFIENGFSDWKIKINWFT |
| | | LEEKQVFDLDFYNNCLLQDWIDNYNKILWWFSEENWNKIQWINEK |
| | | INLFKQNQNKTNSKDVKFPRFKLLYKQILSEKEKLIFVDEIENDEKLINF |
| | | IKESKNNNLIKVEKAIEITENFIKNNETFELDKIYLSKISINTISNKFFSSW |
| | | DYILKEGFDKWEIKEFISFEDLKNAFGKIKYENLEDIFKSNYITDYIAIN |
| | | WEDLYKNFLNIFLYEFKQNINEINFYNSELEKLFLEKFEKTETQVQIIKN |
| | | YFDSVLSLYKMTKYFALEKGKKKIEDLETDNNFYNDFFVYYEDFEIWK |
| | | DYNLVRNFITKKQVKTDKFKLNFENSQFLTGWDKDKEKERLWIILKK |
| | | DEKYYLWILKNNKIFNSYNYESWDFYEKMSYKQLNNVYRQLPRFAF |
| | | SKAKREVYWITPELEQIKEEFDIFQKNKEKWEKFDIEKLKKLINCYKK |
| | | WFIKTYENEFDLEKIKNTDYLDLATFYDEIEQKTYKIDFNKISENFINAK |
| | | VNSWELYLFQIYNKDFSETKKAWSKENIHTKYFKLLFDEKNLEKLVIKL |
| | | SWWAEMFFREKTEKLKTKLDKSWKEVLEHRRYSKDKIMLHLSITLN |
| | | ANKWDSFWFNKMVNEYLNKNEDIKIIWIDRGEKHLAYYSVIDKNW |
| | | KIEEIDTLNIIKSSDWKITNYLEKLEKIESSRKDSRVSWWEIENIKELKN |
| | | GYISQVVNKLAELIIKYNAIIVFEDLNIWFKRWRQKIEKQIYQKLELAL |
| | | AKKLNYLTQKDKNDNEVLWNLKALQLVPKVNDYQDIANYKQSGIM |
| | | FYTRANYTSTTCPCCWFRKNIYISNSDTKEKQKKDFEKIDIKFDGEKFI |
| | | FSYEIIQDKKAKQKSNKTNFSVNSNFSRFKYNSKKMLVEEVNLNLEL |
| | | QNLFKDIDLKWDINKQILEKDSYFYKSLTYYFNLILQLRNSDSKNDIDY |
| | | ITCPSCNYHSKDWFQGLEYNADANWAYNIARKWIIMLDRIEKNFEK |
| | | PDLYVSDIDWDNFTQK |
| 176 | Cas12 | MIKKSNFWQFTGLYELSKTLRFELKPVWETLNILEKDWVSQKDKEVE |
| | | ENYNKIKVFFDSLHREFVKQSLENWYLELLENFYNSYIELNKNIENKK |
| | | NKSLQKLFEKSSKELKKELVSFFEWKWNDWKQKYSFLKKWWIDVL |
| | | NEKEVLDLMWEFYPKEKELFKKFDKFFTYFSNFKESRKNFYADDWR |
| | | AWAIATRVIDENLITFIKNIEDFKKFKNNFSDFIENGFSDWKIKINWFT |
| | | LEEKQVFDLDFYNNCLLQDWIDNYNKILWWFSEENWNKIQWINEK |
| | | INLFKQNQNKTNSKDVKFPRFKLLYKQILSEKEKLIFVDEIENDEKLINF |
| | | IKESKNNNLIKVEKAIEITENFIKNNETFELDKIYLSKISINTISNKFFSSW |
| | | DYILKEGFDKWEIKEFISFEDLKNAFGKIKYENLEDIFKSNYITDYIAIN |
| | | WEDLYKNFLNIFLYEFKQNINEINFYNSELEKLFLEKFEKTETQVQIIKN |
| | | YFDSVLSLYKMTKYFALEKGKKKIEDLETDNNFYNDFFVYYEDFEIWK |
| | | DYNLVRNFITKKQVKTDKFKLNFENSQFLTGWDKDKEKERLWIILKK |
| | | DEKYYLWILKNNKIFNSYNYESWDFYEKMSYKQLNNVYRQLPRFAF |
| | | SKAKREVYWITPELEQIKEEFDIFQKNKEKWEKFDIEKLKKLINCYKK |
| | | WFIKTYENEFDLEKIKNTDYLDLATFYDEIEQKTYKIDFNKISENFINAK |
| | | VNSWELYLFQIYNKDFSETKKAWSKENIHTKYFKLLFDEKNLEKLVIKL |
| | | SWWAEMFFREKTEKLKTKLDKSWKEVLEHRRYSKDKIMLHLSITLN |
| | | ANKWDSFWFNKMVNEYLNKNEDIKIIWIDRGEKHLAYYSVIDKNW |
| | | KIEEIDTLNIIKSSDWKITNYLEKLEKIESSRKDSRVSWWEIENIKELKN |
| | | GYISQVVNKLAELIIKYNAIIVFEDLNIWFKRWRQKIEKQIYQKLELAL |
| | | AKKLNYLTQKDKNDNEVLWNLKALQLVPKVNDYQDIANYKQSGIM |
| | | FYTRANYTSTTCPCCWFRKNIYISNSDTKEKQKKDFEKIDIKFDGEKFI |
| | | FSYEIIQDKKAKQKSNKTNFSVNSNFSRFKYNSKKMLVEEVNLNLEL |
| | | QNLFKDIDLKWDINKQILEKDSYFYKSLTYYFNLILQLRNSDSKNDIDY |
| | | ITCPSCNYHSKDWFQGLEYNADANWAYNIARKWIIMLDRIEKNFEK |
| | | PDLYVSDIDWDNFTQK |
| 177 | Cas12 | MATRSFILKIEPNEEVKKGLWKTHEVLNHGIAYYMNILKLIRQEAIYE |
| | | HHEQDPKNPKKVSKAEIQAELWDFVLKMQKCNSFTHEVDKDVVFN |
| | | ILRELYEELVPSSVEKKGEANQLSNKFLYPLVDPNSQSGKGTASSGRK |
| | | PRWYNLKIAGDPSWEEEKKKWEEDKKKDPLAKILGKLAEYGLIPLFIP |
| | | FTDSNEPIVKEIKWMEKSRNQSVRRLDKDMFIQALERFLSWESWNL |
| | | KVKEEYEKVEKEHKTLEERIKEDIQAFKSLEQYEKERQEQLLRDTLNT |
| | | NEYRLSKRGLRGWREIIQKWLKMDENEPSEKYLEVFKDYQRKHPRE |
| | | AGDYSVYEFLSKKENHFIWRNHPEYPYLYATFCEIDKKKKDAKQQAT |
| | | FTLADPINHPLWVRFEERSGSNLNKYRILTEQLHTEKLKKKLTVQLDR |
| | | LIYPTESGGWEEKGKVDIVLLPSRQFYNQIFLDIEEKGKHAFTYKDESI |
| | | KFPLKGTLGGARVQFDRDHLRRYPHKVESGNVGRIYFNMTVNIEPT |
| | | ESPVSKSLKIHRDDFPKFVNFKPKELTEWIKDSKGKKLKSGIESLEIGLR |
| | | VMSIDLGQRQAAAASIFEVVDQKPDIEGKLFFPIKGTELYAVHRASE |
| | | NIKLPGETLVKSREVLRKAREDNLKLMNQKLNFLRNVLHFQQFEDIT |
| | | EREKRVTKWISRQENSDVPLVYQDELIQIRELMYKPYKDWVAFLKQL |
| | | HKRLEVEIGKEVKHWRKSLSDGRKGLYGISLKNIDEIDRTRKFLLRWS |
| | | LRPTEPGEVRRLEPGQRFAIDQLNHLNALKEDRLKKMANTIIMHAL |
| | | GYCYDVRKKKWQAKNPACQIILFEDLSNYNPYEERSRFENSKLMKW |
| | | SRREIPRQVALQGEIYGLQVGEVGAQFSSRFHAKTGSPGIRCSVVTK |
| | | EKLQDNRFFKNLQREGRLTLDKIAVLKEGDLYPDKGGEKFISLSKDRK |
| | | LVTTHADINAAQNLQKRFWTRTHGFYKVYCKAYQVDGQTVYIPESK |
| | | DQKQKIIEEFGEGYFILKDGVYEWGNAGKLKIKKGSSKQSSSELVDSD |
| | | ILKDSFDLASELKGEKLMLYRDPSGNVFPSDKWMAAGVFFGKLERIL |
| | | ISKLTNQYSISTIEDDSSKQSM |
| 178 | Cas12 | MAESMYNQQNINQKSTETPTEEDALGIKNPLLTPTTLGQNFLSLQSI |
| | | SPLGSRSINLINWSLISPNQTLLQNFQDNENWQDSSISEFRPFSKNSL |
| | | TENSPIIEPQSDKTLPSSVPIQLSSELPIQQPPETSFIDSESPIPQPPETP |
| | | SSDSESPIGQTENIFPFQIDQKTRRNSVIQNSKSSISHFFQKKSEFPRE |
| | | KISQENVNKLTKKSAENQDISTTEESVINSIETNDLQTINPTDIPNPEIP |
| | | VSTLQKQPDSTIDNIPQTESVSTINPTDIPXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | xXXXXxxxxxxxxxxXXXXXXXXXXXXXXXSKNNRIPQLITFLKLNQY |
| | | QQLILQIFLTQKFSFQLSKNNRIPPLITFLKLNQYQQLIPRVLLHQKFLF |
| | | QLFKNNRIPPLITFPKLNQYQQLIPRIFLTQKFPFQISRNNRIPQLITFP |
| | | KLNQYQ |
| 179 | Cas13 | MGAIKNKHIFAAYANLAIDGLIKTLNFIAKKLDTQKQLSSWDIKHVIT |
| | | LIDSIFDQNPQNNLEQVVEGYLPWIKPIIEMKTPKKGERQSDKLCIEY |
| | | KTIITAFASLLNDVRNYYTHYYHDPICIYPGGYDIPSSLNCIYDSAINIIK |
| | | ERFQAEEKEMEHLRRYTRKKGRVVLKTEDDHFYYTLANNNDLSEKG |
| | | YAFFISMFLERKYSYLFLKKLSGFKRGDSLQYRLTLEVFTALSTKPPVER |
| | | LRTTKDTKQDRALDILNELSKIPIELYQTLEPKYREMYNETLQPTDAE |
| | | DPYGLPDRSRIRFRSRFEAFALHFLDKQADFKEIGFYTYLGNYFHNGY |
| | | QKTRVDRETKDRYINFQLAGFCKNIQDISAKKLSEALNVKSIDISTDSI |
| | | PDINSFEPYLVQSTPHYIVNGNNIGIKVLPEGKDTYPTIDEKGAKMPI |
| | | ADFWLSKYELPAMLFYTYLRNNNIHKSHCPLSVKDIIERSIHKSTKQK |
| | | HPEERSELMLRRVMKAIFWTDSKLNEVERIKSQKSAFGKRQHEILKA |
| | | GRIAETLVRDMLWLQPSKNNGRDKVTEPNFQAIQVSLAYFGIRRND |
| | | LTEIFTRAGLINSSNPHPFLAQIGTNYTSLIEFYIAYLKERKVYFSRIQKK |
| | | ILQGKLNIQCHPLRDLQREPNKPQDKEEAIFLPRGLFNEAIINCLKKSK |
| | | LKQLIESPTREKSPALNVSYLIQNYFRTYFEDQSQEFYAQPRNYRLFD |
| | | KLSPNKGKSKSYLSLEQRIKKMEELRPSKIPVAEANKLLEKEDRLYRKN |
| | | YNEICDNESIIRLYQIQDILLFMMTKEYLPSDLYNRINKYKLENVKGIL |
| | | NERVSYLIDLNPLKIQGEDIKIKDYGKLFYIHHDTRISSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIGSSFLIKTRNMFLHDKYEAECIKEIS |
| | | DDFVYAKKIIAEFKMKIENIKLEDLSNDSSA |
| 180 | Cas13 | MARDNFLNTIKLLADKMKLGAISGFGKDGNEVNDINKLFGDKNTYA |
| | | NIENVVEFYFPWIKALEGRFSLDKGDRNLNDMKMFYKSVLTAFFTA |
| | | VDSLRNKYTHYSHKDLNIREIKIECTLGGKDYCIGLLNALDCIYDSAVN |
| | | LLKLRFMVGEDEVAHLRRCKAVNKKVVVRTEKDGFYYRLSDNGGVT |
| | | EKGVIFIASMFLNRKYGFLFLKQLEGFKRSDEKRYRLTLEAFLAFSNIK |
| | | PVDRLKSDKLDRASLGLDMLNELTKIPKELSETLSVDCLYKYLASDGE |
| | | DDLRSRIRYQDRFVPLALEFISQSDEFKDFRFYTYVGNYVYKGYIKRLI |
| | | DGTDKERYLSDRLCGFYKSVNDASSDAIAQKYGVEIKDSNEPDYMLP |
| | | DSFRPHVLRATPHFVINNNNIGIKICGNDCLPIVNGKGVESPEPDYW |
| | | LSIYELPAMLFYAYLREKNGKRFKDYKSIRELIEGVEKKADEKNDRDK |
| | | GALMARHIDKEIIWTQTKLDEVKRLEEKKVAAYGKKGRVVLKSGRM |
| | | ADLLAHDMVRLQPATKGSDKITGVNFQALQVSLAYFKRDILADVFS |
| | | RAMLTTGNHRHPFLYRIDVSHCSSLRDFYVAYLGERRKYFEDVAKKI |
| | | AKNKLNTPCHILRRLQREGSGEEAGKDVKPKFLPRGIFTDSIKNCLEQ |
| | | SKLNIYIRNARNDVKPAINAAYLILMYYKEIEKGEFQGFYGEKRRYDIL |
| | | EEGKPLDLAERKKALASIKPAKIDVSEANMPMSKEEHLMRKRYHAV |
| | | CNNESAIRMYQVQDILLLLMAKDIFKKALSEGVMSKKIGLENLNGIF |
| | | DAPVNFVKNFDNIKLTATGIKIKNYGKVCRLGTDFKFNSLIKAFHKVY |
| | | SKSVEMDYSDYLKEEEEFEKYRLNMVKLCREVERGITEDLHLSLDGKS |
| | | HLSFNDDVIKPYNDKYNVFNGDDLTFFINARNMFMHGDYKYECVK |
| | | YVVSEHFKGSLNDVSFAKETYGHFCNLLESMRKKTGLRIDI |
| 181 | Cas13 | MKKKISLKEQRNTKKAENKLKYQKAQAERAAAAQQ.TAAGAESEEN |
| | | PCFDVVKDTKRKALNPLHVEIEAPSAKKSSVKANGLKSLLLTDGKTV |
| | | MTSFGRGSEANVEKRFDETGTKTFDRDPELFSAKPLETGYRIQRFNA |
| | | SPKDAGLAYRPAGVRPDQIGAKAALEKRYFGKETPGDNIHVQIAYQI |
| | | QDIEKLLAVYISNIIYAVNNVTGVSAMKDSKGRPVDLLGDYGILGEEG |
| | | LTKRLQRIPEQADEEAKALQAFLCSERLSYFGKEFCLVRNSPKQPDKE |
| | | EKRQYKLMRVLCLLGELRQFLVHGKKKEKEFAWLYRLDRQLSQEYR |
| | | KLLGEFYDAQVDKVNKSFLTNSTVNLEVLFRALKTGTDPERKTVTQE |
| | | YYQFTVRKEDGNLGFSLKTLREILLSAYKHEVRDKEYDSIRHKLYQLFS |
| | | FALYHYYKTGVGAERREAFVAKLRAVMTAEAKQRAYADEAAEIWN |
| | | DEGSGIRAAFLEILEAVDFGSAVKGIKARSSVAGDKRFAEWLEEVRIR |
| | | PEGVSCFTKLMYLLTRFLDGKEINELLTGLINKLENIQSFLDVMQQEH |
| | | AETGLSDAFSFFEYSGEIAAELRMTRSFARMAAADPEAKRFMVVDG |
| | | AKLLGFNPKDTESEDEGIIRAIYGDACAEYLQFSEEEKEAFYVQEGLY |
| | | GKEREKFSPYAYFHTDTSLRNFIAKNVVESARFRYVIRYVSPEIARKYA |
| | | RQEALVRFALHRVPLLQLRRYYQSCCGPKKDPDAAECVDFLAGVVN |
| | | RVDFANFTDVRTGDSSKSEQEKKQKYQAIVGLYLTVVYWIVKNLVN |
| | | VNSRYVMAFHILERDTVLLEGKRLFVGGMKAEDPFLLTDGYVSRQD |
| | | AYVRKRIGENKRANRHGLNCVLENRNALGSDPASTDAAASLIWSYR |
| | | NAAAHLTAVAAAQEYVSELREIHSYFEVYHYAMQRYLKSGAEFAELV |
| | | SKNGPASGKIAAWANAVDRCHSFCKDWLWLLNVPFAYNPARYKN |
| | | LSIANLFDKNEAAPVTEDASEQKEDE |
| 182 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KHLIESPTREKSPALNVSYLIQNYFRAYFEDQSQEFYAQPRNYRLFDN |
| | | LSPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDFSNDSSA |
| 183 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPRGYDIPSSLNCIYDSAINIIKE |
| | | RFQAEEKEMEHLRNYTLVNNNGLSEKGYAFFISKFLERKYSYLFLKKL |
| | | SGFKRGDSLQYRLTLEVFTALSTKPPVERLRTTKDTKQDRALDILNELS |
| | | RIPIELYQTLEPKYREMYNETLQPTDAEDPYGLPDRSRIRFRSRFEAFA |
| | | LHFLDKQADFKEIGFYTYLGNYFHNGYQKTRVDRETKDRYINFQLAG |
| | | FCKNIQDISAKKLSEALNVKSIDISTDSIPDINSFEPYLVQSTPHYIVNG |
| | | NNIGIKVLPEGKDTYPTIDEKGAKMPIADFWLSKYELPAMLFYTYLR |
| | | NNNIHKSHCPLSVKDIIERSIHKSTKQKHPEERSELMLRRVMKAIFWT |
| | | DSKLNEVERIKSQKSAFGKRQHEILKAGRIAETLVRDMLWLQPSKNN |
| | | GRDKVTEPNFQAIQVSLAYFGIRRNDLTEIFTRAGLINSSNPHPFLAQ |
| | | IGTNYTSLIEFYIAYLKERKVYFSRIQKKILQGKLNIQCHPLRDLQREPN |
| | | KPQEKEEAIFLPRGLFNEAIINCLKKSKLKHLIESPTREKSPALNVSYLIH |
| | | NYFRAYFEDQSQEFYAQPRNYRLFDKLSPNKGKSKSYLSLEQRIKKM |
| | | EELRPSKIPVAEANKLLEKEDRLYRKNYNEICDNESIIRLYQIQDILLFM |
| | | MTKEYLPSDLYNRINKYKLENVKGILNERVSYLIDLNPLKIQGEDIKIK |
| | | DYGKLFYIHHDTRISSLNKVLSKVKRNNSISSSVKIQPYENYKRECLDF |
| | | EEAQIQIIPIIHSFEIAMVSMFPDLKKATPGNYYDFNELITEYEKRTKQ |
| | | KIDSSFLIKTRNMFLHDKYEAECIKEISDDFVYAKKIIAEFKMKIENIKLE |
| | | DLSNDSSA |
| 184 | Cas13 | CILDFFTQDKAIAEYQLGVEFLQKNLPVIRYLYLPTSHKRFENVPKNQ |
| | | LISEQRNYFKNSLKVLKNLIRDYRNFYTHHFHKPIPVFPETYKLLDDLF |
| | | LAVANDVKKHRMKTDASKQLLKKGLIEELAQLEKLKLEDLKKLKREG |
| | | KKVNLNDKEAITNAILNDSFSHLLPKENTISKYYSAVPTEDIDTENGVT |
| | | ISESGIIFLLGLFLTKKQSEDLRSRVKGFKAKLIVNPENPINKKNNSLKY |
| | | MATHWVFGYLGFKGLKNRFTTTFTKDTLLAQIVDELSKVPDELYQVL |
| | | PEELKNEFLEDMNEYLKEENSESLDKATVIHPVIRKRYENKFAYFALR |
| | | FLDEFVDFPTLRFQLHLGNYVHDKREKPIEGTKYVTERIVKEKIKAFAK |
| | | LSEAAQLKQKYFEEKENHQSIGLQLYPNPSYNFVGNNIPIHLNLNEH |
| | | FFPKEVKIVAGRLKKRNSSYKSDHPEEYKVRTDNKIKPDAILQDLGKP |
| | | EKLAPVAMLSLNELPALLHLVLTKKTPEEIEIIIAQKIAERYNVLTNYKA |
| | | GDDISKGQITKNLLKAKQKKEVNLDKLQLAIEKEIAVTNDKLQTIALHI |
| | | KERNDPKQKRKYVFTNKEIGLQVTWLANDLKRFMPKGSRQNWRG |
| | | QHHSQLQKSLAFYDIQPKEPLSLLEEVWDFKNEAYLWNNGIRRSFD |
| | | KRDFISFYTSYLNNRKETFQRFKDQLNGIRSNKKILDKFIKQQHLWNL |
| | | FHKRLYVIDTIEEQVEKLLVKPMQFPKGVFDHKPTYIKGKSIQENPEC |
| | | FADWYVAWNQHTDYQKFYSWDRDYKSAYLSGEQEKTEKRFIRVQ |
| | | GSKINKVKQQDVLLAKMASIIFNELYLPEDAEHLDLNLSDIYKTQTER |
| | | KAEIEAALIQSHKTTGDNSANIIKSTSAWTLTVPYCSKNIYEPQVKLKE |
| | | LGKFKKFIASQKVQTLFEYKPQKIWNKTELEEVLELKANSYEVIRRDYL |
| | | LKSIQEFEKYMIKKLPTLIDTNEHPNFNKYLTTFLKSLELVSEEDAKWLI |
| | | SKKDFDTTPIDELKKQSKIMEKAFLLVMIRNKFSHNQLPRKIYYDEIYK |
| | | NVPNAVSINFNELFLEYTNQTILEFK |
| 185 | Cas13 | NRRDMEASPGRLARYIVLKTLYERAFPRWLEAREAETLNGWIGRAA |
| | | DRATVAARHINKDENAAARMAGLVRLADGKGIADFTDRLAAETAS |
| | | EYRVQRGYDSDPAVARKQAKHIEDLRCDVVGQAFEAYLADVARKL |
| | | AWTMNDPPGGPLPDEKKASLETAAPPAEDAGAEAEEDWLARLYFL |
| | | LHMLPVEEVGGLRHQLRKWSVLEREPDSVLEREPDADVEAIERILGL |
| | | YLDMHDAKFEGGDGVAGAEALTDLFTSPAQFRRVCPEDGAGNGG |
| | | HVPWRGLREMLRFGGGEPRLMWAFRKWPIGDGMVDGLNALEAT |
| | | VAEAHKQREDLHAKWAGKKGFSRKDKDEYRAALETVVVHRHLAAH |
| | | VQLVNHARLHRLAMAVLARLADYAGLWERDLYFTTLALIRLENGKP |
| | | EDVFRSRELEWLREGRIVDALKELKKNADGSEPAVGTALQRLFGKGI |
| | | LTGGGTVSVRRDLLHFNMLQRKANEPFNLTTAVNDTRKLMAYDRK |
| | | LKNAVSRSIIELLAREGLDLAWEMKDHQLAGAVLKTRQAVHLGGAK |
| | | VGGGPITEDLHGPEFTAMAAALFGGEARAATDAAEAATGRPDRRE |
| | | RKSGGASPRPARPAKTLPAPTPRLLPAAGERVEGVLVEEKTRKGGW |
| | | KAAVEIGGPRIVGDIFNSGEVPSDAEPGLEAEFVVRVANPANASFM |
| | | WLSPDVEERLKKAAAPRRGGRPAGRQR |
| 186 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KHLIESPTREKSPALNVSYLIQNYFRAYFEDQSQEFYAQPRNYRLFDN |
| | | LSPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDFSNDSSA |
| 187 | Cas13 | MGAIENKHIFAAYANLAIDGLIKTLNFIAKKLDTQKQLSSWDIKFMTL |
| | | IDSIFDQNPQNNLEQVVEGYLPWIKPIIEMKTPKKGERQSDKLCIEYK |
| | | TIITAFASLLNDVRNYYTHYYHDPICIYPRGYDIPSSLNCIYDSAINIIKE |
| | | RFQAEEKEMEHLRNYTLVNNNGLSEKGYAFFISKFLERKYSYLFLKKL |
| | | SGFKRGDSLQYRLTLEVFTALSTKPPVERLRTTKDTKQDRALDILNELS |
| | | RIPIELYQTLEPKYREMYNETLQPTDAEDPYGLPDRSRIRFRSRFEAFA |
| | | LHFLDKQADFKEIGFYTYLGNYFHNGYQKTRVDRETKDRYINFQLAG |
| | | FCKNIQDISAKKLSEALNVKSIDISTDSIPDINSFEPYLVQSTPHYIVNG |
| | | NNIGIKVLPEGKDTYPTIDEKGAKMPIADFWLSKYELPAMLFYTYLR |
| | | NNNIHKSHCPLSVKDIIERSIHKSTKQKHPEERSELMLRRVMKAIFWT |
| | | DSKLNEVERIKSQKSAFGKRQHEILKAGRIAETLVRDMLWLQPSKNN |
| | | GRDKVTEPNFQAIQVSLAYFGIRRNDLTEIFTRAGLINSSNPHPFLAQ |
| | | IGTNYTSLIEFYIAYLKERKVYFSRIQKKILQGKLNIQCHPLRDLQREPN |
| | | KPQEKEEAIFLPRGLFNEAIINCLKKSKLKHLIESPTREKSPALNVSYLIH |
| | | NYFRAYFEDQSQEFYAQPRNYRLFDKLSPNKGKSKSYLSLEQRIKKM |
| | | EELRPSKIPVAEANKLLEKEDRLYRKNYNEICDNESIIRLYQIQDILLFM |
| | | MTKEYLPSDLYNRINKYKLENVKGILNERVSYLIDLNPLKIQGEDIKIK |
| | | DYGKLFYIHHDTRISSLNKVLSKVKRNNSISSSVKIQPYENYKRECLDF |
| | | EEAQIQIIPIIHSFEIAMVSMFPDLKKATPGNYYDFNELITEYEKRTKQ |
| | | KIDSSFLIKTRNMFLHDKYEAECIKEISDDFVYAKKIIAEFKMKIENIKLE |
| | | DLSNDSSA |
| 188 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KHLIESPTREKSPALNVSYLIQNYFRAYFEDQSQEFYAQPRNYRLFDN |
| | | LSPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDFSNDSSA |
| 189 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPRGYDIPSSLNCIYDSAINIIKE |
| | | RFQAEEKEMEHLRNYTLVNNNGLSEKGYAFFISKFLERKYSYLFLKKL |
| | | SGFKRGDSLQYRLTLEVFTALSTKPPVERLRTTKDTKQDRALDILNELS |
| | | RIPIELYQTLEPKYREMYNETLQPTDAEDPYGLPDRSRIRFRSRFEAFA |
| | | LHFLDKQADFKEIGFYTYLGNYFHNGYQKTRVDRETKDRYINFQLAG |
| | | FCKNIQDISAKKLSEALNVKSIDISTDSIPDINSFEPYLVQSTPHYIVNG |
| | | NNIGIKVLPEGKDTYPTIDEKGAKMPIADFWLSKYELPAMLFYTYLR |
| | | NNNIHKSHCPLSVKDIIERSIHKSTKQKHPEERSELMLRRVMKAIFWT |
| | | DSKLNEVERIKSQKSAFGKRQHEILKAGRIAETLVRDMLWLQPSKNN |
| | | GRDKVTEPNFQAIQVSLAYFGIRRNDLTEIFTRAGLINSSNPHPFLAQ |
| | | IGTNYTSLIEFYIAYLKERKVYFSRIQKKILQGKLNIQCHPLRDLQREPN |
| | | KPQEKEEAIFLPRGLFNEAIINCLKKSKLKHLIESPTREKSPALNVSYLIH |
| | | NYFRAYFEDQSQEFYAQPRNYRLFDKLSPNKGKSKSYLSLEQRIKKM |
| | | EELRPSKIPVAEANKLLEKEDRLYRKNYNEICDNESIIRLYQIQDILLFM |
| | | MTKEYLPSDLYNRINKYKLENVKGILNERVSYLIDLNPLKIQGEDIKIK |
| | | DYGKLFYIHHDTRISSLNKVLSKVKRNNSISSSVKIQPYENYKRECLDF |
| | | EEAQIQIIPIIHSFEIAMVSMFPDLKKATPGNYYDFNELITEYEKRTKQ |
| | | KIDSSFLIKTRNMFLHDKYEAECIKEISDDFVYAKKIIAEFKMKIENIKLE |
| | | DLSNDSSA |
| 190 | Cas13 | MEEANRYIYGAYFNMARDNFLNTIKLLADKMKLGATSGFGKDGNE |
| | | VNDINELFGDKNTYANIENVVEFYFPWIKALEGRFSLDKGDRNLND |
| | | MKMFYKSVLTAFFTAVDSLRNKYTHYSHKDLNIREIKIECTLGGKDYC |
| | | IGLLNALDCIYDSAVNLLKLRFMAGEYEVAHLRRCKAVNKKVVVRTE |
| | | KDGFYYRLSDNGGVTEKGVIFIASMFLNRKYGFLFLKQLEGFKRSDEK |
| | | RYRLTLETFLAFSNIKPVDRLKSDKLDRASLGLDMLNELTKIPRELSETL |
| | | SVDCLYKYLTSDGEDDLRSRIRYQDRFVPLALEFISQSDEFKDFRFYTY |
| | | VGNYVYKGYIKRLIDGTDKERYLSDRLCGFYKSVNDASSDAIAQKYG |
| | | VEIKDSNEPDYMLPDSFRPHVLRATPHFVINNNNIGIKICGNDCLPIV |
| | | NGKGVESPEPDYWLSIYELPAMLFYAYLREKNGKLLKDYKSIRELIED |
| | | VEKKADEKNDRDKGALMARHIDKEIIWTQTKLDEVKRLEEKKVAAY |
| | | GKKGRVVLKSGRMADLLAHDMVRLQPATKGSDKITGANFQALQVS |
| | | LAYFKRDILADVFSRAMLTTGNHRHPFLYRIDVSHCSSLRDFYVAYLG |
| | | ERRKYFEDVAKKIAKNKLNTPCHILRRLQREGSGEEAGKDVKPKFLPR |
| | | GIFTGSIKSCLEKSALNINIRNARNDVKPAINAAYLILMYYKEIEKGEFQ |
| | | GFYGEKRRYDILEEGKPLDLDERKKALASIKPAKIDVSEANMPMSKEE |
| | | HLMRKRYHAVCNNESAIRMYQVQDILLLLMAKDIFKKALSEGVMSK |
| | | KIGLENLNGIFDAPVNFVKNFDNIKLTATGIKIKDYGKVCRLGTDFKF |
| | | NSLVKAFHKVYSKSVEMDYSDYLKEEEEFEKYRLNMVKLCREVERGI |
| | | TEDLHLSLDGKSHLGFNDDVIKPYNDKYNVFNGGDLTFFINARNMF |
| | | MHGDYKYECVKYVVSEHFKGSLNDVSFAKETYGHFCNLLESMRKKT |
| | | GLRIDI |
| 191 | Cas13 | MGAIKNKHIFAAYANLAIDGLIKTLNFIAKKLDTQKQLSSWDIKHVIT |
| | | LIDSIFDQNPQNNLEQVVEGYLPWIKPIIEMKTPKKGERQSDKLCIEY |
| | | KTIITAFASLLNDVRNYYTHYYHDPICIYPGGYDIPSSLNCIYDSAINIIK |
| | | ERFQAEEKEMEHLRRYTRKKGRVVLKTEDDHFYYTLANNNDLSEKG |
| | | YAFFISMFLERKYSYLFLKKLSGFKRGDSLQYRLTLEVFTALSTKPPVER |
| | | LRTTKDTKQDRALDILNELSKIPIELYQTLEPKYREMYNETLQPTDAE |
| | | DPYGLPDRSRIRFRSRFEAFALHFLDKQADFKEIGFYTYLGNYFHNGY |
| | | QKTRVDRETKDRYINFQLAGFCKNIQDISAKKLSEALNVKSIDISTDSI |
| | | PDINSFEPYLVQSTPHYIVNGNNIGIKVLPEGKDTYPTIDEKGAKMPI |
| | | ADFWLSKYELPAMLFYTYLRNNNIHKSHCPLSVKDIIERSIHKSTKQK |
| | | HPEERSELMLRRVMKAIFWTDSKLNEVERIKSQKSAFGKRQHEILKA |
| | | GRIAETLVRDMLWLQPSKNNGRDKVTEPNFQAIQVSLAYFGIRRND |
| | | LTEIFTRAGLINSSNPHPFLAQIGTNYTSLIEFYIAYLKERKVYFSRIQKK |
| | | ILQGKLNIQCHPLRDLQREPNKPQDKEEAIFLPRGLFNEAIINCLKKSK |
| | | LKQLIESPTREKSPALNVSYLIQNYFRTYFEDQSQEFYAQPRNYRLFD |
| | | KLSPNKGKSKSYLSLEQRIKKMEELRPSKIPVAEANKLLEKEDRLYRKN |
| | | YNEICDNESIIRLYQIQDILLFMMTKEYLPSDLYNRINKYKLENVKGIL |
| | | NERVSYLIDLNPLKIQGEDIKIKDYGKLFYIHHDTRISSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIGSSFLIKTRNMFLHDKYEAECIKEIS |
| | | DDFVYAKKIIAEFKMKIENIKLEDLSNDSSA |
| 192 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KHLIESPTREKSPALNVSYLIQNYFRAYFEDQSQEFYAQPRNYRLFDN |
| | | LSPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDFSNDSSA |
| 193 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEIEHLRRYTRKKGRVVLKTEDDHFYYTLVNNNDLSEKGYA |
| | | FFISMFLERKYSYLFLKKLSGFKRGDSLQYRLTLEVFTALSTKPPVERLR |
| | | TTKDTKQDRALDILNELSRIPIELYQTLEPKYREMYNETLQPTDAEDP |
| | | YGLPDRSRIRFRSRFEAFALHFLDKQADFKEIGFYTYLGNYFHNGYQK |
| | | TRVDRETKDRYINFQLAGFCKNIQDISAKKLSEALNVKSIDISTDSIPDI |
| | | NSFEPYLVQSTPHYIVNGNNIGIKVLPEGKDTYPTIDEKGAKMPIADF |
| | | WLSKYELPAMLFYTYLRNNNIHKSHCPLSVKDIIERSIHKSTKQKHPE |
| | | ERSELMLRRVMKAIFWTDSKLNEVERIKSQKSAFGKRQHEILKAGRI |
| | | AETLVRDMLWLQPSKNNGRDKVTEPNFQAIQVSLAYFGIRRNDLTE |
| | | IFTRAGLINSSNPHPFLAQIGTNYTSLIEFYIAYLKERKVYFSRIQKKILQ |
| | | GKLNIQCHPLRDLQREPNKPQDKEEAIFLPRGLFNEAIINCLKKSKLK |
| | | QLIESPTREKSPALNVSYLIQNYFRTYFEDQSQEFYAQPRNYRLFDKL |
| | | SPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDLSNDSSA |
| 194 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KHLIESPTREKSPALNVSYLIQNYFRAYFEDQSQEFYAQPRNYRLFDN |
| | | LSPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDFSNDSSA |
| 195 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLQPTDAED |
| | | PYGLPDRSRIRFRSRFETFALHFLDKQPDFKEIGFYTYLGNYFHNGYQ |
| | | KTRVDRETKDRYINFQLAGFCKNIQDISAKKLSEALNVKSIDISTDSIP |
| | | DINSFEPYLVQSMPHYIVNGNNIGIKVLPEGKDTYPTIDEKGAKMPI |
| | | ADFWLSKYELPAMLFYTYLRNNNIHKSHCPLSVKDIIERSIHKSTKQK |
| | | HPEERSELMLRRVMKAIFWTDSKLNEVERIKSQKSAFGKRQHEILKA |
| | | GRIAEMLVRDMLWLQPSKNNGRDKVTEPNFQAIQVSLAYFGIRRN |
| | | DLTEIFTRAGLINSSNPHPFLAQIGTNYTSLIEFYIAYLKERKVYFSRIQK |
| | | KILQGKLNIQCHPLRDLQREPNKPQEKEEAIFLPRGLFNEAIINCLKKS |
| | | KLKQLIESPTREKSPALNVSYLIQNYFRAYFEDQSQEFYAQPRNYRLF |
| | | DKLSPNKGKSKSYLSLEQRIKKMEELRPSKIPVAEANKLLEKEDRLYRK |
| | | NYNEICDNESIIRLYQIQDILLFMMTKEYLPSDLYNRINKYKLENVKGI |
| | | LNERVSYLIDLNPLKIQGEDIKIKDYGKLFYIHHDTRISSLNKVLSKVKR |
| | | NNSISSSVKIQPYENYKRECLDFEEAQIQIIPIIHSFEIAMVSMFPDLKK |
| | | ATPGNYYDFNELITEYEKRTKQKIDSSFLIKTRNMFLHDKYEAECIKEIS |
| | | DDFVYAKKIIAEFKMKIENIKLEDLSNDSSA |
| 196 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPRGYDIPSSLNCIYDSAINIIKE |
| | | RFQAEEKEMEHLRNYTLVNNNGLSEKGYAFFISKFLERKYSYLFLKKL |
| | | SGFKRGDSLQYRLTLEVFTALSTKPPVERLRTTKDTKQDRALDILNELS |
| | | RIPIELYQTLEPKYREMYNETLQPTDAEDPYGLPDRSRIRFRSRFEAFA |
| | | LHFLDKQADFKEIGFYTYLGNYFHNGYQKTRVDRETKDRYINFQLAG |
| | | FCKNIQDISAKKLSEALNVKSIDISTDSIPDINSFEPYLVQSTPHYIVNG |
| | | NNIGIKVLPEGKDTYPTIDEKGAKMPIADFWLSKYELPAMLFYTYLR |
| | | NNNIHKSHCPLSVKDIIERSIHKSTKQKHPEERSELMLRRVMKAIFWT |
| | | DSKLNEVERIKSQKSAFGKRQHEILKAGRIAETLVRDMLWLQPSKNN |
| | | GRDKVTEPNFQAIQVSLAYFGIRRNDLTEIFTRAGLINSSNPHPFLAQ |
| | | IGTNYTSLIEFYIAYLKERKVYFSRIQKKILQGKLNIQCHPLRDLQREPN |
| | | KPQEKEEAIFLPRGLFNEAIINCLKKSKLKHLIESPTREKSPALNVSYLIH |
| | | NYFRAYFEDQSQEFYAQPRNYRLFDKLSPNKGKSKSYLSLEQRIKKM |
| | | EELRPSKIPVAEANKLLEKEDRLYRKNYNEICDNESIIRLYQIQDILLFM |
| | | MTKEYLPSDLYNRINKYKLENVKGILNERVSYLIDLNPLKIQGEDIKIK |
| | | DYGKLFYIHHDTRISSLNKVLSKVKRNNSISSSVKIQPYENYKRECLDF |
| | | EEAQIQIIPIIHSFEIAMVSMFPDLKKATPGNYYDFNELITEYEKRTKQ |
| | | KIDSSFLIKTRNMFLHDKYEAECIKEISDDFVYAKKIIAEFKMKIENIKLE |
| | | DLSNDSSA |
| 197 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEIEHLRRYTRKKGRVVLKTEDDHFYYTLVNNNDLSEKGYA |
| | | FFISMFLERKYSYLFLKKLSGFKRGDSLQYRLTLEVFTALSTKPPVERLR |
| | | TTKDTKQDRALDILNELSRIPIELYQTLEPKYREMYNETLQPTDAEDP |
| | | YGLPDRSRIRFRSRFEAFALHFLDKQADFKEIGFYTYLGNYFHNGYQK |
| | | TRVDRETKDRYINFQLAGFCKNIQDISAKKLSEALNVKSIDISTDSIPDI |
| | | NSFEPYLVQSTPHYIVNGNNIGIKVLPEGKDTYPTIDEKGAKMPIADF |
| | | WLSKYELPAMLFYTYLRNNNIHKSHCPLSVKDIIERSIHKSTKQKHPE |
| | | ERSELMLRRVMKAIFWTDSKLNEVERIKSQKSAFGKRQHEILKAGRI |
| | | AETLVRDMLWLQPSKNNGRDKVTEPNFQAIQVSLAYFGIRRNDLTE |
| | | IFTRAGLINSSNPHPFLAQIGTNYTSLIEFYIAYLKERKVYFSRIQKKILQ |
| | | GKLNIQCHPLRDLQREPNKPQDKEEAIFLPRGLFNEAIINCLKKSKLK |
| | | QLIESPTREKSPALNVSYLIQNYFRTYFEDQSQEFYAQPRNYRLFDKL |
| | | SPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDLSNDSSA |
| 198 | Cas13 | MGAIENKHIFAAYANLAIDGLIKTLNFIAKKLDTQKQLSSWDIKFMTL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSRIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KQLIESPTREKSPALNVSYLILNYFRTYFEDQSQEFYAQPRNYRLFDKL |
| | | SPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRINSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIDSSFLIKTRNMFLHDKYEAECIKEIS |
| | | DDFVYAKKIIAEFKMKIENIKLEDFSNDSSA |
| 199 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KHLIESPTREKSPALNVSYLIQNYFRAYFEDQSQEFYAQPRNYRLFDK |
| | | LSPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDLSNDSSA |
| 200 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSKKGY |
| | | AFFISMFLERKYSYLFLKKLSGFKRGDSLQYRLTLEVFTALSTKPPVERL |
| | | RTTKDTKQDRALDILNELSRIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KQLIESPTREKSPALNVSYLILNYFRTYFEDQSQEFYAQPRNYRLFDKL |
| | | SPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRINSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIDSSFLIKTRNMFLHDKYEAECIKEIS |
| | | DDFVYAKKIIAEFKMKIENIKLEDFSNDSSA |
| 201 | Cas13 | MKHVFAAYFSDARLNIMASLNDVREKSGLKRYKNEAENVQNFEGIF |
| | | PKNIASDIRDKRITLLRIRFPFIETIVDPGRGKENPASKYGELSWLFQLV |
| | | NDMRNVFIHSTGSEEEIDYQHHKKIFNALRKVYDCGLRTVKSRFQLE |
| | | NDTTMPLLRCDNRGRPKPFNKFSLALCTSPERNGNKKQSDVLHDFG |
| | | RVLLCSLFLEKRQISGLVSHFWDKNGYGTDWNDSEQTIIRELLYVNRI |
| | | RLPSQRLRTDSTLTSVTLDTISELARCPRPLFELLDADGQENFRVGRS |
| | | PKNPQNRNDDPSYLLLRGHQSRFIPLAMRHLDFDSKCKLRFAVDLG |
| | | QYYHSVRLKPAESFIDGNPGIRRLGQKIIAFGRLKDFEDAEKPEIWKK |
| | | LEENGQKFAEEEEELLKRASITGSPEELKPYIIKTFPHYHLYRDKIGFCID |
| | | WEEKKDQKVKYPNLGVRGEDSKSTDSGGIKKRENRQLSRHQFWISP |
| | | NKIIDLAFYHYLQTEIKQEITASVKTGRKKCPYPSVENILKEYYEGMW |
| | | LIKELKEQGPLPPWTEIPQISERRAECHRWINKEIIKAENFTISLADLPK |
| | | AIRRHLKVLDSRETSVSDIIKRTKTMIEETCKKQKEIEYLLKYPKKRGKK |
| | | GFRPIKHGNIADFLTEDLLRFQPKDSSKKNGGKLTSKNYQILQKAIAY |
| | | YDKPYCIVDLLKKSGLLEGEFKHPFLCKIITEENHEQYSTLIDFYQKYLE |
| | | ERKAFLEGFIDTFVAGSSIPGWLRLRKPSTFESWLDQQLDEDEKLCQ |
| | | PLPVPKSLFYQMLLKMTAEKLDLTPEMLCKKGTQRFIRDSQEVEVKP |
| | | SVSWFIRQYMEHRDDTAQEMYMFKRRHELFDFFHDKKKTTKKLHE |
| | | EKTCCLSEKVRQEELESVHKGIEKLKKLLRTYKRSEKKIRHFSTMDMV |
| | | LYLLAKKNFEKLILCDETSGPDWSLKTLETKILSTKIKYELNVPGTDRTI |
| | | VHRACAIKKTGELRLLVRDRRLPSLLDYYPKTEKSIDQEEIKVELADYS |
| | | CKRIEAMKLISDLEQKIEESCNRGFCKPVPDKLKKIFGAKKHGTLLYAL |
| | | YHRFHEEKDKSVDEQGFNKDCFAHARQIRNAFAHNEYPVSEQFNQI |
| | | VRQVNETSQPHNHNSPVNHPKIADQLVKELDKLYKPWRNFLKRIAG |
| 202 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPRGYDIPSSLNCIYDSAINIIKE |
| | | RFQAEEKEMEHLRNYTLVNNNGLSEKGYAFFISKFLERKYSYLFLKKL |
| | | SGFKRGDSLQYRLTLEVFTALSTKPPVERLRTTKDTKQDRALDILNELS |
| | | RIPIELYQTLEPKYREMYNETLQPTDAEDPYGLPDRSRIRFRSRFEAFA |
| | | LHFLDKQADFKEIGFYTYLGNYFHNGYQKTRVDRETKDRYINFQLAG |
| | | FCKNIQDISAKKLSEALNVKSIDISTDSIPDINSFEPYLVQSTPHYIVNG |
| | | NNIGIKVLPEGKDTYPTIDEKGAKMPIADFWLSKYELPAMLFYTYLR |
| | | NNNIHKSHCPLSVKDIIERSIHKSTKQKHPEERSELMLRRVMKAIFWT |
| | | DSKLNEVERIKSQKSAFGKRQHEILKAGRIAETLVRDMLWLQPSKNN |
| | | GRDKVTEPNFQAIQVSLAYFGIRRNDLTEIFTRAGLINSSNPHPFLAQ |
| | | IGTNYTSLIEFYIAYLKERKVYFSRIQKKILQGKLNIQCHPLRDLQREPN |
| | | KPQEKEEAIFLPRGLFNEAIINCLKKSKLKHLIESPTREKSPALNVSYLIH |
| | | NYFRAYFEDQSQEFYAQPRNYRLFDKLSPNKGKSKSYLSLEQRIKKM |
| | | EELRPSKIPVAEANKLLEKEDRLYRKNYNEICDNESIIRLYQIQDILLFM |
| | | MTKEYLPSDLYNRINKYKLENVKGILNERVSYLIDLNPLKIQGEDIKIK |
| | | DYGKLFYIHHDTRISSLNKVLSKVKRNNSISSSVKIQPYENYKRECLDF |
| | | EEAQIQIIPIIHSFEIAMVSMFPDLKKATPGNYYDFNELITEYEKRTKQ |
| | | KIDSSFLIKTRNMFLHDKYEAECIKEISDDFVYAKKIIAEFKMKIENIKLE |
| | | DLSNDSSA |
| 203 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPRGYDIPSSLNCIYDSAINIIKE |
| | | RFQAEEKEMEHLRNYTLVNNNGLSEKGYAFFISKFLERKYSYLFLKKL |
| | | SGFKRGDSLQYRLTLEVFTALSTKPPVERLRTTKDTKQDRALDILNELS |
| | | RIPIELYQTLEPKYREMYNETLQPTDAEDPYGLPDRSRIRFRSRFEAFA |
| | | LHFLDKQADFKEIGFYTYLGNYFHNGYQKTRVDRETKDRYINFQLAG |
| | | FCKNIQDISAKKLSEALNVKSIDISTDSIPDINSFEPYLVQSTPHYIVNG |
| | | NNIGIKVLPEGKDTYPTIDEKGAKMPIADFWLSKYELPAMLFYTYLR |
| | | NNNIHKSHCPLSVKDIIERSIHKSTKQKHPEERSELMLRRVMKAIFWT |
| | | DSKLNEVERIKSQKSAFGKRQHEILKAGRIAETLVRDMLWLQPSKNN |
| | | GRDKVTEPNFQAIQVSLAYFGIRRNDLTEIFTRAGLINSSNPHPFLAQ |
| | | IGTNYTSLIEFYIAYLKERKVYFSRIQKKILQGKLNIQCHPLRDLQREPN |
| | | KPQEKEEAIFLPRGLFNEAIINCLKKSKLKHLIESPTREKSPALNVSYLIH |
| | | NYFRAYFEDQSQEFYAQPRNYRLFDKLSPNKGKSKSYLSLEQRIKKM |
| | | EELRPSKIPVAEANKLLEKEDRLYRKNYNEICDNESIIRLYQIQDILLFM |
| | | MTKEYLPSDLYNRINKYKLENVKGILNERVSYLIDLNPLKIQGEDIKIK |
| | | DYGKLFYIHHDTRISSLNKVLSKVKRNNSISSSVKIQPYENYKRECLDF |
| | | EEAQIQIIPIIHSFEIAMVSMFPDLKKATPGNYYDFNELITEYEKRTKQ |
| | | KIDSSFLIKTRNMFLHDKYEAECIKEISDDFVYAKKIIAEFKMKIENIKLE |
| | | DLSNDSSA |
| 204 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KHLIESPTREKSPALNVSYLIQNYFRAYFEDQSQEFYAQPRNYRLFDN |
| | | LSPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDFSNDSSA |
| 205 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPRGYDIPSSLNCIYDSAINIIKE |
| | | RFQAEEKEMEHLRNYTLVNNNGLSEKGYAFFISKFLERKYSYLFLKKL |
| | | SGFKRGDSLQYRLTLEVFTALSTKPPVERLRTTKDTKQDRALDILNELS |
| | | RIPIELYQTLEPKYREMYNETLQPTDAEDPYGLPDRSRIRFRSRFEAFA |
| | | LHFLDKQADFKEIGFYTYLGNYFHNGYQKTRVDRETKDRYINFQLAG |
| | | FCKNIQDISAKKLSEALNVKSIDISTDSIPDINSFEPYLVQSTPHYIVNG |
| | | NNIGIKVLPEGKDTYPTIDEKGAKMPIADFWLSKYELPAMLFYTYLR |
| | | NNNIHKSHCPLSVKDIIERSIHKSTKQKHPEERSELMLRRVMKAIFWT |
| | | DSKLNEVERIKSQKSAFGKRQHEILKAGRIAETLVRDMLWLQPSKNN |
| | | GRDKVTEPNFQAIQVSLAYFGIRRNDLTEIFTRAGLINSSNPHPFLAQ |
| | | IGTNYTSLIEFYIAYLKERKVYFSRIQKKILQGKLNIQCHPLRDLQREPN |
| | | KPQEKEEAIFLPRGLFNEAIINCLKKSKLKHLIESPTREKSPALNVSYLIH |
| | | NYFRAYFEDQSQEFYAQPRNYRLFDKLSPNKGKSKSYLSLEQRIKKM |
| | | EELRPSKIPVAEANKLLEKEDRLYRKNYNEICDNESIIRLYQIQDILLFM |
| | | MTKEYLPSDLYNRINKYKLENVKGILNERVSYLIDLNPLKIQGEDIKIK |
| | | DYGKLFYIHHDTRISSLNKVLSKVKRNNSISSSVKIQPYENYKRECLDF |
| | | EEAQIQIIPIIHSFEIAMVSMFPDLKKATPGNYYDFNELITEYEKRTKQ |
| | | KIDSSFLIKTRNMFLHDKYEAECIKEISDDFVYAKKIIAEFKMKIENIKLE |
| | | DLSNDSSA |
| 206 | Cas13 | MGAIKNKHIFAAYANLAIDGLIKTLNFIAKKLDTQKQLSSWDIKHVIT |
| | | LIDSIFDQNPQNNLEQVVEGYLPWIKPIIEMKTPKKGERQSDKLCIEY |
| | | KTIITAFASLLNDVRNYYTHYYHDPICIYPGGYDIPSSLNCIYDSAINIIK |
| | | ERFQAEEKEMEHLRRYTRKKGRVVLKTEDDHFYYTLANNNDLSEKG |
| | | YAFFISMFLERKYSYLFLKKLSGFKRGDSLQYRLTLEVFTALSTKPPVER |
| | | LRTTKDTKQDRALDILNELSKIPIELYQTLEPKYREMYNETLQPTDAE |
| | | DPYGLPDRSRIRFRSRFEAFALHFLDKQADFKEIGFYTYLGNYFHNGY |
| | | QKTRVDRETKDRYINFQLAGFCKNIQDISAKKLSEALNVKSIDISTDSI |
| | | PDINSFEPYLVQSTPHYIVNGNNIGIKVLPEGKDTYPTIDEKGAKMPI |
| | | ADFWLSKYELPAMLFYTYLRNNNIHKSHCPLSVKDIIERSIHKSTKQK |
| | | HPEERSELMLRRVMKAIFWTDSKLNEVERIKSQKSAFGKRQHEILKA |
| | | GRIAETLVRDMLWLQPSKNNGRDKVTEPNFQAIQVSLAYFGIRRND |
| | | LTEIFTRAGLINSSNPHPFLAQIGTNYTSLIEFYIAYLKERKVYFSRIQKK |
| | | ILQGKLNIQCHPLRDLQREPNKPQDKEEAIFLPRGLFNEAIINCLKKSK |
| | | LKQLIESPTREKSPALNVSYLIQNYFRTYFEDQSQEFYAQPRNYRLFD |
| | | KLSPNKGKSKSYLSLEQRIKKMEELRPSKIPVAEANKLLEKEDRLYRKN |
| | | YNEICDNESIIRLYQIQDILLFMMTKEYLPSDLYNRINKYKLENVKGIL |
| | | NERVSYLIDLNPLKIQGEDIKIKDYGKLFYIHHDTRISSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIGSSFLIKTRNMFLHDKYEAECIKEIS |
| | | DDFVYAKKIIAEFKMKIENIKLEDLSNDSSA |
| 207 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSKKGY |
| | | AFFISMFLERKYSYLFLKKLSGFKRGDSLQYRLTLEVFTALSTKPPVERL |
| | | RTTKDTKQDRALDILNELSRIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KQLIESPTREKSPALNVSYLILNYFRTYFEDQSQEFYAQPRNYRLFDKL |
| | | SPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRINSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIDSSFLIKTRNMFLHDKYEAECIKEIS |
| | | DDFVYAKKIIAEFKMKIENIKLEDFSNDSSA |
| 208 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMKHLRNYTLVNNNGLSEKGYAFFISKFLERKYSYLFLKKL |
| | | SGFKRGDSLQYRLTLEVFTALSTKPPVERLRTTKDTKQDRALDILNELS |
| | | KIPIELYQTLEPKYREMYNETLQPTDAEDPYGLPDRSRIRFRSRFEAFA |
| | | LHFLDKQADFKEIGFYTYLGNYFHNGYQKTRVDRETKDRYINFQLAG |
| | | FCKNIQDISAKKLSEALNVKSIDISTDSIPDINSFEPYLVQSTPHYIVNG |
| | | NNIGIKVLPEGKDTYPTIDEKGAKMPIADFWLSKYELPAMLFYTYLR |
| | | NNNIHKSHCPLSVKDIIERSIHKSTKQKHPEERSELMLRRVMKAIFWT |
| | | DSKLNEVERIKSQKSAFGKRQHEILKAGRIAETLVRDMLWLQPSKNN |
| | | GRDKVTEPNFQAIQVSLAYFGIRRNDLTEIFTRAGLINSSNPHPFLAQ |
| | | IGTNYTSLIEFYIAYLKERKVYFSRIQKKILQGKLNIQCHPLRDLQREPN |
| | | KPQEKEEAIFLPRGLFNEAIINCLKKSKLKQLIESPTREKSPALNVSYLIQ |
| | | NYFRTYFEDQSQEFYAQPRNYRLFDKLSPNKGKSKSYLSLEQRIKKM |
| | | EELRPSKIPVAEANKLLEKEDRLYRKNYNEICDNESIIRLYQIQDILLFM |
| | | MTKEYLPSDLYNRINKYKLENVKGILNERVSYLIDLNPLKIQGEDIKIK |
| | | DYGKLFYIHHDTRINSLNKVLSKVKRNNSISSSVKIQPYENYKRECLDF |
| | | EEAQIQIIPIIHSFEIAMVSMFPDLKKATPGNYYDFNELITEYEKRTKQ |
| | | KIDSSFLIKTRNMFLHDKYEAECIKEISDDLVYAKKIIAEFKMKIENIKLE |
| | | DLSNDSSA |
| 209 | Cas13 | MGAIKNKHIFAAYANLAIDGLIKTLNFIAKKLDTQKQLSSWDIKHVIT |
| | | LIDSIFDQNPQNNLEQVVEGYLPWIKPIIEMKTPKKGERQSDKLCIEY |
| | | KTIITAFASLLNDVRNYYTHYYHDPICIYPGGYDIPSSLNCIYDSAINIIK |
| | | ERFQAEEKEMEHLRRYTRKKGRVVLKTEDDHFYYTLANNNDLSEKG |
| | | YAFFISMFLERKYSYLFLKKLSGFKRGDSLQYRLTLEVFTALSTKPPVER |
| | | LRTTKDTKQDRALDILNELSKIPIELYQTLEPKYREMYNETLQPTDAE |
| | | DPYGLPDRSRIRFRSRFEAFALHFLDKQADFKEIGFYTYLGNYFHNGY |
| | | QKTRVDRETKDRYINFQLAGFCKNIQDISAKKLSEALNVKSIDISTDSI |
| | | PDINSFEPYLVQSTPHYIVNGNNIGIKVLPEGKDTYPTIDEKGAKMPI |
| | | ADFWLSKYELPAMLFYTYLRNNNIHKSHCPLSVKDIIERSIHKSTKQK |
| | | HPEERSELMLRRVMKAIFWTDSKLNEVERIKSQKSAFGKRQHEILKA |
| | | GRIAETLVRDMLWLQPSKNNGRDKVTEPNFQAIQVSLAYFGIRRND |
| | | LTEIFTRAGLINSSNPHPFLAQIGTNYTSLIEFYIAYLKERKVYFSRIQKK |
| | | ILQGKLNIQCHPLRDLQREPNKPQDKEEAIFLPRGLFNEAIINCLKKSK |
| | | LKQLIESPTREKSPALNVSYLIQNYFRTYFEDQSQEFYAQPRNYRLFD |
| | | KLSPNKGKSKSYLSLEQRIKKMEELRPSKIPVAEANKLLEKEDRLYRKN |
| | | YNEICDNESIIRLYQIQDILLFMMTKEYLPSDLYNRINKYKLENVKGIL |
| | | NERVSYLIDLNPLKIQGEDIKIKDYGKLFYIHHDTRISSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIGSSFLIKTRNMFLHDKYEAECIKEIS |
| | | DDFVYAKKIIAEFKMKIENIKLEDLSNDSSA |
| 210 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSRIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KQLIESPTREKSPALNVSYLILNYFRTYFEDQSQEFYAQPRNYRLFDKL |
| | | SPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRINSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIDSSFLIKTRNMFLHDKYEAECIKEIS |
| | | DDFVYAKKIIAEFKMKIENIKLEDFSNDSSA |
| 211 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KHLIESPTREKSPALNVSYLIQNYFRAYFEDQSQEFYAQPRNYRLFDN |
| | | LSPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDFSNDSSA |
| 212 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPRGYDIPSSLNCIYDSAINIIKE |
| | | RFQAEEKEMEHLRNYTLVNNNGLSEKGYAFFISKFLERKYSYLFLKKL |
| | | SGFKRGDSLQYRLTLEVFTALSTKPPVERLRTTKDTKQDRALDILNELS |
| | | RIPIELYQTLEPKYREMYNETLQPTDAEDPYGLPDRSRIRFRSRFEAFA |
| | | LHFLDKQADFKEIGFYTYLGNYFHNGYQKTRVDRETKDRYINFQLAG |
| | | FCKNIQDISAKKLSEALNVKSIDISTDSIPDINSFEPYLVQSTPHYIVNG |
| | | NNIGIKVLPEGKDTYPTIDEKGAKMPIADFWLSKYELPAMLFYTYLR |
| | | NNNIHKSHCPLSVKDIIERSIHKSTKQKHPEERSELMLRRVMKAIFWT |
| | | DSKLNEVERIKSQKSAFGKRQHEILKAGRIAETLVRDMLWLQPSKNN |
| | | GRDKVTEPNFQAIQVSLAYFGIRRNDLTEIFTRAGLINSSNPHPFLAQ |
| | | IGTNYTSLIEFYIAYLKERKVYFSRIQKKILQGKLNIQCHPLRDLQREPN |
| | | KPQEKEEAIFLPRGLFNEAIINCLKKSKLKHLIESPTREKSPALNVSYLIH |
| | | NYFRAYFEDQSQEFYAQPRNYRLFDKLSPNKGKSKSYLSLEQRIKKM |
| | | EELRPSKIPVAEANKLLEKEDRLYRKNYNEICDNESIIRLYQIQDILLFM |
| | | MTKEYLPSDLYNRINKYKLENVKGILNERVSYLIDLNPLKIQGEDIKIK |
| | | DYGKLFYIHHDTRISSLNKVLSKVKRNNSISSSVKIQPYENYKRECLDF |
| | | EEAQIQIIPIIHSFEIAMVSMFPDLKKATPGNYYDFNELITEYEKRTKQ |
| | | KIDSSFLIKTRNMFLHDKYEAECIKEISDDFVYAKKIIAEFKMKIENIKLE |
| | | DLSNDSSA |
| 213 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSRIPIELYQTLEPKYREMYNATLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KQLIESPTREKSPALNVSYLILNYFRTYFEDQSQEFYAQPRNYRLFDKL |
| | | SPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRINSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIDSSFLIKTRNMFLHDKYEAECIKEIS |
| | | DDFVYAKKIIAEFKMKIENIKLEDFSNDSSA |
| 214 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLQPTDAED |
| | | PYGLPDRSRIRFRSRFETFALHFLDKQPDFKEIGFYTYLGNYFHNGYQ |
| | | KTRVDRETKDRYINFQLAGFCKNIQDISAKKLSEALNVKSIDISTDSIP |
| | | DINSFEPYLVQSMPHYIVNGNNIGIKVLPEGKDTYPTIDEKGAKMPI |
| | | ADFWLSKYELPAMLFYTYLRNNNIHKSHCPLSVKDIIERSIHKSTKQK |
| | | HPEERSELMLRRVMKAIFWTDSKLNEVERIKSQKSAFGKRQHEILKA |
| | | GRIAEMLVRDMLWLQPSKNNGRDKVTEPNFQAIQVSLAYFGIRRN |
| | | DLTEIFTRAGLINSSNPHPFLAQIGTNYTSLIEFYIAYLKERKVYFSRIQK |
| | | KILQGKLNIQCHPLRDLQREPNKPQEKEEAIFLPRGLFNEAIINCLKKS |
| | | KLKQLIESPTREKSPALNVSYLIQNYFRAYFEDQSQEFYAQPRNYRLF |
| | | DKLSPNKGKSKSYLSLEQRIKKMEELRPSKIPVAEANKLLEKEDRLYRK |
| | | NYNEICDNESIIRLYQIQDILLFMMTKEYLPSDLYNRINKYKLENVKGI |
| | | LNERVSYLIDLNPLKIQGEDIKIKDYGKLFYIHHDTRISSLNKVLSKVKR |
| | | NNSISSSVKIQPYENYKRECLDFEEAQIQIIPIIHSFEIAMVSMFPDLKK |
| | | ATPGNYYDFNELITEYEKRTKQKIDSSFLIKTRNMFLHDKYEAECIKEIS |
| | | DDFVYAKKIIAEFKMKIENIKLEDLSNDSSA |
| 215 | Cas13 | MGAIKNKHIFAAYANLAIDGLIKTLNFIAKKLDTQKQLSSWDIKHVIT |
| | | LIDSIFDQNPQNNLEQVVEGYLPWIKPIIEMKTPKKGERQSDKLCIEY |
| | | KTIITAFASLLNDVRNYYTHYYHDPICIYPGGYDIPSSLNCIYDSAINIIK |
| | | ERFQAEEKEMEHLRRYTRKKGRVVLKTEDDHFYYTLANNNDLSEKG |
| | | YAFFISMFLERKYSYLFLKKLSGFKRGDSLQYRLTLEVFTALSTKPPVER |
| | | LRTTKDTKQDRALDILNELSKIPIELYQTLEPKYREMYNETLQPTDAE |
| | | DPYGLPDRSRIRFRSRFEAFALHFLDKQADFKEIGFYTYLGNYFHNGY |
| | | QKTRVDRETKDRYINFQLAGFCKNIQDISAKKLSEALNVKSIDISTDSI |
| | | PDINSFEPYLVQSTPHYIVNGNNIGIKVLPEGKDTYPTIDEKGAKMPI |
| | | ADFWLSKYELPAMLFYTYLRNNNIHKSHCPLSVKDIIERSIHKSTKQK |
| | | HPEERSELMLRRVMKAIFWTDSKLNEVERIKSQKSAFGKRQHEILKA |
| | | GRIAETLVRDMLWLQPSKNNGRDKVTEPNFQAIQVSLAYFGIRRND |
| | | LTEIFTRAGLINSSNPHPFLAQIGTNYTSLIEFYIAYLKERKVYFSRIQKK |
| | | ILQGKLNIQCHPLRDLQREPNKPQDKEEAIFLPRGLFNEAIINCLKKSK |
| | | LKQLIESPTREKSPALNVSYLIQNYFRTYFEDQSQEFYAQPRNYRLFD |
| | | KLSPNKGKSKSYLSLEQRIKKMEELRPSKIPVAEANKLLEKEDRLYRKN |
| | | YNEICDNESIIRLYQIQDILLFMMTKEYLPSDLYNRINKYKLENVKGIL |
| | | NERVSYLIDLNPLKIQGEDIKIKDYGKLFYIHHDTRISSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIGSSFLIKTRNMFLHDKYEAECIKEIS |
| | | DDFVYAKKIIAEFKMKIENIKLEDLSNDSSA |
| 216 | Cas13 | MGKLYGYKRWYEIEQNNGEKGSFKRKIRVKRVYDEESKSYVVRRDE |
| | | VEDTELIKNKNFIINLKDYKTRNSNIQKFYDKFHVGNILFKLKASVKKG |
| | | KKTFYKDLEKAETVLTNVEILEQ.TIKLANNSKINRKKLEKELTQLNINEK |
| | | SEEVTIDNIKIYLRQGEKFDKKENINAIKWRKLTSEELTIKVEIYRECQSI |
| | | NSNLYSLLDYVLSNEEYDDRYYLEEVENKLLIFGTGNKNKNGRNYYYF |
| | | DYVLKSLSKIKGLIKKDDRNLNYLMFLFNIQKTSENKEHFINKIFNYFK |
| | | YEVRIEKEDIVEFLVGELEYYDLIKRIEKKPSENQNQTNLEKTYILLDKH |
| | | EKLKETIDTKNEIVQKLTIELKNNNLRNRIEIILHKYKILELVDKLNKNIK |
| | | NGKINTELYGIYKEHYGQCIEYINFNELALEEKELYKIIYRYLKGRIEKLL |
| | | QNRNKIKIGELRIEDIFIFQKLLEKIEIRVKQYLLEHILYLGKLKHHNIDE |
| | | VNTIRFVEEHANEELSLELITLFSATNVELNRLLKVKGENGSYEKDYDF |
| | | FSAKPDKGKIKIKDTNSVMKFELLKKLKFINSEATETNQEAIDFLKEAY |
| | | NLRNNILHGKNEEIIEDNKKNLSKSYKNINELIEELRPSDNEICKSLNLD |
| | | IIFKGNRKINDINAKLFGNNREKIYLPSFSKLVPEIKNIIESYDKNGTFN |
| | | DERIKKIVLNGAIYVNKILYLKECSNKDGEFIKNLKEELNKDSKEKSKYV |
| | | SIEELYKKSQISASKGNKKAIYKYQRKIIEIYLKYLKENYVEILDFSKLNLN |
| | | IEQIENDIKNRKNSENKVLIESIKQKVFPENDFEYIISIFALLNDNIFINKI |
| | | RNRFFSTDTWLKNNRYSNIIKILDEVISVNLLRTELLNTSIDIEEIKDDV |
| | | LTEDIENIIPEIREEILQRTKKDFKTLLGNNAISKEGLSNEDIDKIRNIED |
| | | AKINLDFVNNEIKVSLKEYGSLPPNGVLSRNTSKYYNNEIAKKIDQISIL |
| | | TFTKKSIGTISDEKFRNIYWQERKESDESKKIFVYNKNILYLVTKHSFEK |
| | | LYKNFLEEELNNLKLEDTKYLRDLDLRREKNLKVDNTLKEINEKVRGY |
| | | SKEYKKKFIENLKNNDEYFGKVVSGRFKNYQEFKEIYDEVSEYKKIRD |
| | | VVNFNPLNKVYNYLIEINWKLAIQMARAERDLHYIVNGLNELKLIEL |
| | | NQGQNDGISRAYPKYKLNKEKNKKELRLEECHYNFDIDNYKKFEKIC |
| | | EKLGIDLSENGELQQENETNIRNYISHFYILRKPFVDISISEAIKRVSKLL |
| | | SYRTRYNNSTYSSVFEVFKKDVELNYDFLKKKIELNGKTYDEVVQRKK |
| | | ISCLELESYLDYKPIIKKILF |
| 217 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KHLIESPTREKSPALNVSYLIQNYFRAYFEDQSQEFYAQPRNYRLFDN |
| | | LSPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDFSNDSSA |
| 218 | Cas13 | MGAIKNKHIFAAYANLAIDGLIKTLNFIAKKLDTQKQLSSWDIKHVIT |
| | | LIDSIFDQNPQNNLEQVVEGYLPWIKPIIEMKTPKKGERQSDKLCIEY |
| | | KTIITAFASLLNDVRNYYTHYYHDPICIYPGGYDIPSSLNCIYDSAINIIK |
| | | ERFQAEEKEMEHLRRYTRKKGRVVLKTEDDHFYYTLANNNDLSEKG |
| | | YAFFISMFLERKYSYLFLKKLSGFKRGDSLQYRLTLEVFTALSTKPPVER |
| | | LRTTKDTKQDRALDILNELSKIPIELYQTLEPKYREMYNETLQPTDAE |
| | | DPYGLPDRSRIRFRSRFEAFALHFLDKQADFKEIGFYTYLGNYFHNGY |
| | | QKTRVDRETKDRYINFQLAGFCKNIQDISAKKLSEALNVKSIDISTDSI |
| | | PDINSFEPYLVQSTPHYIVNGNNIGIKVLPEGKDTYPTIDEKGAKMPI |
| | | ADFWLSKYELPAMLFYTYLRNNNIHKSHCPLSVKDIIERSIHKSTKQK |
| | | HPEERSELMLRRVMKAIFWTDSKLNEVERIKSQKSAFGKRQHEILKA |
| | | GRIAETLVRDMLWLQPSKNNGRDKVTEPNFQAIQVSLAYFGIRRND |
| | | LTEIFTRAGLINSSNPHPFLAQIGTNYTSLIEFYIAYLKERKVYFSRIQKK |
| | | ILQGKLNIQCHPLRDLQREPNKPQDKEEAIFLPRGLFNEAIINCLKKSK |
| | | LKQLIESPTREKSPALNVSYLIQNYFRTYFEDQSQEFYAQPRNYRLFD |
| | | KLSPNKGKSKSYLSLEQRIKKMEELRPSKIPVAEANKLLEKEDRLYRKN |
| | | YNEICDNESIIRLYQIQDILLFMMTKEYLPSDLYNRINKYKLENVKGIL |
| | | NERVSYLIDLNPLKIQGEDIKIKDYGKLFYIHHDTRISSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIGSSFLIKTRNMFLHDKYEAECIKEIS |
| | | DDFVYAKKIIAEFKMKIENIKLEDLSNDSSA |
| 219 | Cas13 | MGAIKNKHIFAAYANLAIDGLIKTLNFIAKKLDTQKQLSSWDIKHVIT |
| | | LIDSIFDQNPQNNLEQVVEGYLPWIKPIIEMKTPKKGERQSDKLCIEY |
| | | KTHTAFASLLNDVRNYYTHYYHDPICIYPGGYDIPSSLNCIYDSAINIIK |
| | | ERFQAEEKEMEHLRRYTRKKGRVVLKTEDDHFYYTLANNNDLSEKG |
| | | YAFFISMFLERKYSYLFLKKLSGFKRGDSLQYRLTLEVFTALSTKPPVER |
| | | LRTTKDTKQDRALDILNELSKIPIELYQTLEPKYREMYNETLQPTDAE |
| | | DPYGLPDRSRIRFRSRFEAFALHFLDKQADFKEIGFYTYLGNYFHNGY |
| | | QKTRVDRETKDRYINFQLAGFCKNIQDISAKKLSEALNVKSIDISTDSI |
| | | PDINSFEPYLVQSTPHYIVNGNNIGIKVLPEGKDTYPTIDEKGAKMPI |
| | | ADFWLSKYELPAMLFYTYLRNNNIHKSHCPLSVKDIIERSIHKSTKQK |
| | | HPEERSELMLRRVMKAIFWTDSKLNEVERIKSQKSAFGKRQHEILKA |
| | | GRIAETLVRDMLWLQPSKNNGRDKVTEPNFQAIQVSLAYFGIRRND |
| | | LTEIFTRAGLINSSNPHPFLAQIGTNYTSLIEFYIAYLKERKVYFSRIQKK |
| | | ILQGKLNIQCHPLRDLQREPNKPQDKEEAIFLPRGLFNEAIINCLKKSK |
| | | LKQLIESPTREKSPALNVSYLIQNYFRTYFEDQSQEFYAQPRNYRLFD |
| | | KLSPNKGKSKSYLSLEQRIKKMEELRPSKIPVAEANKLLEKEDRLYRKN |
| | | YNEICDNESIIRLYQIQDILLFMMTKEYLPSDLYNRINKYKLENVKGIL |
| | | NERVSYLIDLNPLKIQGEDIKIKDYGKLFYIHHDTRISSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIGSSFLIKTRNMFLHDKYEAECIKEIS |
| | | DDFVYAKKIIAEFKMKIENIKLEDLSNDSSA |
| 220 | Cas13 | MGKLYGYKRWYEIEQNNGEKGSFKRKIRVKRVYDEESKSYVVRRDE |
| | | VEDTELIKNKNFIINLKDYKTRNSNIQKFYDKFHVGNILFKLKASVKKG |
| | | KKTFYKDLEKAETVLTNVEILEQTIKLANNSKINRKKLEKELTQLNINEK |
| | | SEEVTIDNIKIYLRQGEKFDKKENINAIKWRKLTSEELTIKVEIYRECQSI |
| | | NSNLYSLLDYVLSNEEYDDRYYLEEVENKLLIFGTGNKNKNGRNYYYF |
| | | DYVLKSLSKIKGLIKKDDRNLNYLMFLFNIQKTSENKEHFINKIFNYFK |
| | | YEVRIEKEDIVEFLVGELEYYDLIKRIEKKPSENQNQTNLEKTYILLDKH |
| | | EKLKETIDTKNEIVQKLTIELKNNNLRNRIEIILHKYKILELVDKLNKNIK |
| | | NGKINTELYGIYKEHYGQCIEYINFNELALEEKELYKIIYRYLKGRIEKLL |
| | | QNRNKIKIGELRIEDIFIFQKLLEKIEIRVKQYLLEHILYLGKLKHHNIDE |
| | | VNTIRFVEEHANEELSLELITLFSATNVELNRLLKVKGENGSYEKDYDF |
| | | FSAKPDKGKIKIKDTNSVMKFELLKKLKFINSEATETNQEAIDFLKEAY |
| | | NLRNNILHGKNEEIIEDNKKNLSKSYKNINELIEELRPSDNEICKSLNLD |
| | | IIFKGNRKINDINAKLFGNNREKIYLPSFSKLVPEIKNIIESYDKNGTFN |
| | | DERIKKIVLNGAIYVNKILYLKECSNKDGEFIKNLKEELNKDSKEKSKYV |
| | | SIEELYKKSQISASKGNKKAIYKYQRKIIEIYLKYLKENYVEILDFSKLNLN |
| | | IEQIENDIKNRKNSENKVLIESIKQKVFPENDFEYIISIFALLNDNIFINKI |
| | | RNRFFSTDTWLKNNRYSNIIKILDEVISVNLLRTELLNTSIDIEEIKDDV |
| | | LTEDIENIIPEIREEILQRTKKDFKTLLGNNAISKEGLSNEDIDKIRNIED |
| | | AKINLDFVNNEIKVSLKEYGSLPPNGVLSRNTSKYYNNEIAKKIDQISIL |
| | | TFTKKSIGTISDEKFRNIYWQERKESDESKKIFVYNKNILYLVTKHSFEK |
| | | LYKNFLEEELNNLKLEDTKYLRDLDLRREKNLKVDNTLKEINEKVRGY |
| | | SKEYKKKFIENLKNNDEYFGKVVSGRFKNYQEFKEIYDEVSEYKKIRD |
| | | VVNFNPLNKVYNYLIEINWKLAIQMARAERDLHYIVNGLNELKLIEL |
| | | NQGQNDGISRAYPKYKLNKEKNKKELRLEECHYNFDIDNYKKFEKIC |
| | | EKLGIDLSENGELQQENETNIRNYISHFYILRKPFVDISISEAIKRVSKLL |
| | | SYRTRYNNSTYSSVFEVFKKDVELNYDFLKKKIELNGKTYDEVVQRKK |
| | | ISCLELESYLDYKPIIKKILF |
| 221 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEIEHLRRYTRKKGRVVLKTEDDHFYYTLVNNNDLSEKGYA |
| | | FFISMFLERKYSYLFLKKLSGFKRGDSLQYRLTLEVFTALSTKPPVERLR |
| | | TTKDTKQDRALDILNELSRIPIELYQTLEPKYREMYNETLQPTDAEDP |
| | | YGLPDRSRIRFRSRFEAFALHFLDKQADFKEIGFYTYLGNYFHNGYQK |
| | | TRVDRETKDRYINFQLAGFCKNIQDISAKKLSEALNVKSIDISTDSIPDI |
| | | NSFEPYLVQSTPHYIVNGNNIGIKVLPEGKDTYPTIDEKGAKMPIADF |
| | | WLSKYELPAMLFYTYLRNNNIHKSHCPLSVKDIIERSIHKSTKQKHPE |
| | | ERSELMLRRVMKAIFWTDSKLNEVERIKSQKSAFGKRQHEILKAGRI |
| | | AETLVRDMLWLQPSKNNGRDKVTEPNFQAIQVSLAYFGIRRNDLTE |
| | | IFTRAGLINSSNPHPFLAQIGTNYTSLIEFYIAYLKERKVYFSRIQKKILQ |
| | | GKLNIQCHPLRDLQREPNKPQDKEEAIFLPRGLFNEAIINCLKKSKLK |
| | | QLIESPTREKSPALNVSYLIQNYFRTYFEDQSQEFYAQPRNYRLFDKL |
| | | SPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFRIKTRNMFLHDKYEAECIKEISD |
| | | DFVYAKKIIAEFKMKIENIKLEDLSNDSSA |
| 222 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KHLIESPTREKSPALNVSYLIQNYFRAYFEDQSQEFYAQPRNYRLFDN |
| | | LSPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDFSNDSSA |
| 223 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYIRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSRIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KQLIESPTREKSPVLNVSYLIQNYFRAYFEDQSQEFYAQPRNYRLFDK |
| | | LSPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDFSNDSSA |
| 224 | Cas13 | MGAIENKHIFAAYANLAIDGLIKTLNFIAKKLDTQKQLSSWDIKFMTL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMKHLRNYTLVNNNGLSEKGYAFFISKFLERKYSYLFLKKL |
| | | SGFKRGDSLQYRLTLEVFTALSTKPPVERLRTTKDTKQDRALDILNELS |
| | | KIPIELYQTLEPKYREMYNETLQPTDAEDPYGLPDRSRIRFRSRFEAFA |
| | | LHFLDKQADFKEIGFYTYLGNYFHNGYQKTRVDRETKDRYINFQLAG |
| | | FCKNIQDISAKKLSEALNVKSIDISTDSIPDINSFEPYLVQSTPHYIVNG |
| | | NNIGIKVLPEGKDTYPTIDEKGAKMPIADFWLSKYELPAMLFYTYLR |
| | | NNNIHKSHCPLSVKDIIERSIHKSTKQKHPEERSELMLRRVMKAIFWT |
| | | DSKLNEVERIKSQKSAFGKRQHEILKAGRIAETLVRDMLWLQPSKNN |
| | | GRDKVTEPNFQAIQVSLAYFGIRRNDLTEIFTRAGLINSSNPHPFLAQ |
| | | IGTNYTSLIEFYIAYLKERKVYFSRIQKKILQGKLNIQCHPLRDLQREPN |
| | | KPQEKEEAIFLPRGLFNEAIINCLKKSKLKQLIESPTREKSPALNVSYLIQ |
| | | NYFRTYFEDQSQEFYAQPRNYRLFDKLSPNKGKSKSYLSLEQRIKKM |
| | | EELRPSKIPVAEANKLLEKEDRLYRKNYNEICDNESIIRLYQIQDILLFM |
| | | MTKEYLPSDLYNRINKYKLENVKGILNERVSYLIDLNPLKIQGEDIKIK |
| | | DYGKLFYIHHDTRINSLNKVLSKVKRNNSISSSVKIQPYENYKRECLDF |
| | | EEAQIQIIPIIHSFEIAMVSMFPDLKKATPGNYYDFNELITEYEKRTKQ |
| | | KIDSSFLIKTRNMFLHDKYEAECIKEISDDLVYAKKIIAEFKMKIENIKLE |
| | | DLSNDSSA |
| 225 | Cas13 | MGAIKNKHIFAAYANLAIDGLIKTLNFIAKKLDTQKQLSSWDIKHVIT |
| | | LIDSIFDQNPQNNLEQVVEGYLPWIKPIIEMKTPKKGERQSDKLCIEY |
| | | KTIITAFASLLNDVRNYYTHYYHDPICIYPGGYDIPSSLNCIYDSAINIIK |
| | | ERFQAEEKEMEHLRRYTRKKGRVVLKTEDDHFYYTLANNNDLSEKG |
| | | YAFFISMFLERKYSYLFLKKLSGFKRGDSLQYRLTLEVFTALSTKPPVER |
| | | LRTTKDTKQDRALDILNELSKIPIELYQTLEPKYREMYNETLQPTDAE |
| | | DPYGLPDRSRIRFRSRFEAFALHFLDKQADFKEIGFYTYLGNYFHNGY |
| | | QKTRVDRETKDRYINFQLAGFCKNIQDISAKKLSEALNVKSIDISTDSI |
| | | PDINSFEPYLVQSTPHYIVNGNNIGIKVLPEGKDTYPTIDEKGAKMPI |
| | | ADFWLSKYELPAMLFYTYLRNNNIHKSHCPLSVKDIIERSIHKSTKQK |
| | | HPEERSELMLRRVMKAIFWTDSKLNEVERIKSQKSAFGKRQHEILKA |
| | | GRIAETLVRDMLWLQPSKNNGRDKVTEPNFQAIQVSLAYFGIRRND |
| | | LTEIFTRAGLINSSNPHPFLAQIGTNYTSLIEFYIAYLKERKVYFSRIQKK |
| | | ILQGKLNIQCHPLRDLQREPNKPQDKEEAIFLPRGLFNEAIINCLKKSK |
| | | LKQLIESPTREKSPALNVSYLIQNYFRTYFEDQSQEFYAQPRNYRLFD |
| | | KLSPNKGKSKSYLSLEQRIKKMEELRPSKIPVAEANKLLEKEDRLYRKN |
| | | YNEICDNESIIRLYQIQDILLFMMTKEYLPSDLYNRINKYKLENVKGIL |
| | | NERVSYLIDLNPLKIQGEDIKIKDYGKLFYIHHDTRISSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIGSSFLIKTRNMFLHDKYEAECIKEIS |
| | | DDFVYAKKIIAEFKMKIENIKLEDLSNDSSA |
| 226 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSRIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KQLIESPTREKSPALNVSYLILNYFRTYFEDQSQEFYAQPRNYRLFDKL |
| | | SPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRINSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIDSSFLIKTRNMFLHDKYEAECIKEIS |
| | | DDFVYAKKIIAEFKMKIENIKLEDFSNDSSA |
| 227 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KHLIESPTREKSPALNVSYLIQNYFRAYFEDQSQEFYAQPRNYRLFDN |
| | | LSPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDFSNDSSA |
| 228 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KHLIESPTREKSPALNVSYLIQNYFRAYFEDQSQEFYAQPRNYRLFDN |
| | | LSPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDFSNDSSA |
| 229 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 230 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQ.TNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 231 | Cas13 | MDTPNFSERIPVSLQSHPYYFAHYLNMARHNAYVILEYVNRELIKPG |
| | | KNLDEDNLIQSTVLKDGYFDRKPDELSHRNRLLVQHFPFLREAENEG |
| | | ARTCNPVSYKLKTALAALNQWRNNASHYPLNQNHEKDFDLQPFFS |
| | | FAIEACKKRMREVFQPDDFYLLETNEKQFYTLHNENGFTEKGLYCFIC |
| | | FFLEKKYAFQFLAGIKGFKNTTDNKFRATLETFTEHCCRLPKPKLDSS |
| | | DIKLDMLGELSRCPAPLFDLLDIEERKKFIREPEEVKPDESGDREEVQ |
| | | QVLMKRYDDRFPYFALRYFEEKNLLKGISFHIHIGRWIKSEHTKKIMG |
| | | AERDRRLLKDIRTFGELKEFSPEHAPDYWLRDGITPDDVDQFSPQYR |
| | | IVGNRIGIKLNYNGHNRWSVPDKEINVKPDAIISTYEFLNLFLYEHLY |
| | | QKKLTGLSPAEFIQDYLDRFNNFLSEFKAGHIRPVGDFSLEKRRGQG |
| | | DEPDLTARRKSLQKELDRFVLKGKDLPDKIREYLLGYKQKSEKKQAK |
| | | WILGGMIKETVYWRNKAEQSPEKMRSGDMAQQLARDIIFLTPPHT |
| | | VKEHKQKLNSLEYDVLQYALAYFSSNREKLYSFFKEHQLTVKGDRAH |
| | | PFLYKIRLDECQGILDFFIVYMQQKEKWLGWLDRNLKSPRLNEEEFF |
| | | NTYSYFIKTDTKRAIEMDYESCPNYLPRGIFNEPIAKALQKAGVKIKDE |
| | | DNASYALSVYSNGKTQPFYNKERYYNKGIFRMEELPEKLQPKELLGKI |
| | | QWTIKSSGKDTEEFRSLQNLKNRILNTEKEIRYVQSTDRALWIMVAD |
| | | LFPETFELRPDDLECIGHDLSDDLLSRPYQMKEKVYNYTITDYLPIKRY |
| | | GEFRRFLKDRRLENLLTYFEEGVPLHREALVAELEAYDLQRKNLLEIIY |
| | | RFEKLVFDRHRHELTFSGEGENQYVNHWDYLDFVARKYGLSAEVKE |
| | | LNSERFTELRNKMLHNQIPYQLWIKEAIAAREENTVCGRIMGMIGEI |
| | | YERMTTEIEKQMQV |
| 232 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 233 | Cas13 | MENKTSLGNNIYYNPFKPQDKSYFAGYFNAAMENTDSVFRELGKRL |
| | | KGKEYTSENFFDAIFKENISLVEYERYVKLLSDYFPMARLLDKKEVPIK |
| | | ERKENFKKNFKGIIKAVRDLRNFYTHKEHGEVEITDEIFGVLDEMLKS |
| | | TVLTVKKKKVKTDKTKEILKKSIEKQLDILCQKKLEYLRDTARKIEEKRR |
| | | NQRERGEKELVAPFKYSDKRDDLIAAIYNDAFDVYIDKKKDSLKESSK |
| | | AKYNTKSDPQQEEGDLKIPISKNGVVFLLSLFLTKQEIHAFKSKIAGFK |
| | | ATVIDEATVSEATVSHGKNSICFMATHEIFSHLAYKKLKRKVRTAEIN |
| | | YGEAENAEQLSVYAKETLMMQMLDELSKVPDVVYQNLSEDVQKTF |
| | | IEDWNEYLKENNGDVGTMEEEQVIHPVIRKRYEDKFNYFAIRFLDEF |
| | | AQFPTLRFQVHLGNYLHDSRPKENLISDRRIKEKITVFGRLSELEHKKA |
| | | LFIKNTETNEDREHYWEIFPNPNYDFPKENISVNDKDFPIAGSILDRE |
| | | KQPVAGKIGIKVKLLNQQYVSEVDKAVKAHQLKQRKASKPSIQNIIEE |
| | | IVPINESNPKEAIVFGGQPTAYLSMNDIHSILYEFFDKWEKKKEKLEK |
| | | KGEKELRKEIGKELEKKIVGKIQAQIQQIIDKDTNAKILKPYQDGNSTA |
| | | IDKEKLIKDLKQEQNILQKLKDEQTVREKEYNDFIAYQDKNREINKVR |
| | | DRNHKQYLKDNLKRKYPEAPARKEVLYYREKGKVAVWLANDIKRF |
| | | MPTDFKNEWKGEQHSLLQKSLAYYEQCKEELKNLLPEKVFQHLPFK |
| | | LGGYFQQKYLYQFYTCYLDKRLEYISGLVQQAENFKSENKVFKKVEN |
| | | ECFKFLKKQNYTHKELDARVQSILGYPIFLERGFMDEKPTIIKGKTFKG |
| | | NEALFADWFRYYKEYQNFQTFYDTENYPLVELEKKQADRKRKTKIYQ |
| | | QKKNDVFTLLMAKHIFKSVFKQDSIDQFSLEDLYQSREERLGNQERA |
| | | RQTGERNTNYIWNKTVDLKLCDGKITVENVKLKNVGDFIKYEYDQR |
| | | VQAFLKYEENIEWQAFLIKESKEEENYPYVVEREIEQYEKVRREELLKE |
| | | VHLIEEYILEKVKDKEILKKGDNQNFKYYILNGLLKQLKNEDVESYKVF |
| | | NLNTEPEDVNINQLKQEATDLEQKAFVLTYIRNKFAHNQLPKKEFW |
| | | DYCQEKYGKIEKEKTYAEYFAEVFKKEKEALIK |
| 234 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQ.TNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 235 | Cas13 | MTEQSERPYNGTYYTLEDKHFWAAFLNLARHNAYITLTHIDRQLAYS |
| | | KADITNDQDVLSFKALWKNLDNDLERKSRLRSLILKHFSFLEGAAYG |
| | | KKLFESKSSGNKSSKNKELTKKEKEELQANALSLDNLKSILFDFLQKLK |
| | | DFRNYYSHYRHSGSSELPLFDGNMLQRLYNVFDVSVQRVKRDHEH |
| | | NDKVDPHYHFNHLVRKGKKDRYGHNDNPSFKHHFVDSEGMVTEA |
| | | GLLFFVSLFLEKRDAIWMQKKIRGFKGGTGPYEQMTNEVFCRSRISL |
| | | PKLKLESLRTDDWMLLDMLNELVRCPKPLYDRLREKDRACFRVPVDI |
| | | LPDEDDTDGGGEDPFKNTLVRHQDRFPYFALRYFDLKKVFTSLRFHI |
| | | DLGTYHFAIYKKMIGEQPEDRHLTRNLYGFGRIQDFAEEHRPEEWK |
| | | RLVRDLDYLETGDKPYISQTTPHYHIEKGKIGLRFVPEGQHLWPSPEV |
| | | GTTRTGRSKCAQDKRLTAEAFLSVHELMPMMFYYFLLREKYSEEVS |
| | | AEKVQGRIKRVIEDVYAIYDAFARDEINTLKELDTCLADKGIRRGHLP |
| | | KQMITILSQERKDMKEKIRKKLQEMIADTDHRLDMLDRQTDRKIRI |
| | | GRKNAGLPKSGVIADWLVRDMMRFQPVAKDASGKPLNNSKANST |
| | | EYRMLQRALALFGGEKERLTPYFRQMNLTGGNNPHPFLHETRWES |
| | | HTNILSFYRSYLRARKAFLERIGRSDRVENCPFLLLKEPKTDRQTLVAG |
| | | WKDEFHLPRGIFTEAVRDCLIEMGYDEVGSYREVGFMAKAVPLYFE |
| | | RACEDRVQPFYDSPFNVGNSLKPKKGRFLSKEDRAEEWERGMERF |
| | | RDLEAWSHSAARRIKDAFAGIEYASPGNKKKIEQLLRDLSLWEAFES |
| | | KLKVRADKINLAKLKKEILEAQEHPYHDFKSWQKFERELRLVKNQDII |
| | | TWMMCRDLMEENKVEGLDTGTLYLKDIRPNVQEQGSLNVLNRVK |
| | | PMRLPVVVYRADSRGHVHKEAPLATVYIEERNTKLLKQGNFKSFVK |
| | | DRRLNGLFSFVDTGGLAMEQYPISKLRVEYELAKYQTARVCVFELTL |
| | | RLEESLLSRYPHLPDESFREMLESWSDPLLAKWPELHGKVRLLIAVR |
| | | NAFSHNQYPMYDEAVFSSIRKYDPSSPDAIEERMGLNIAHRLSEEVK |
| | | QAKETVERIIQA |
| 236 | Cas13 | MEKPLPPNVYTLKHKFFWGAFLNIARHNAFITICHINEQLGLTTPPN |
| | | DDKIADVVCGTWNNILNNDHDLLKKSQLTELILKHFPFLAAMCYHP |
| | | PKKEGKKKGSQKEQQKEKENEAQSQAEALNPSELIKALKTLVKQLRT |
| | | LRNYYSHYKHKKPDAEKDIFKHLYKAFDASLRMVKEDYKAHFTVNLT |
| | | QDFAHLNRKGKNKQDNPKFDRYRFEKDGFFTESGLLFFTNLFLDKR |
| | | DAYWMLKKVSGFKASHKQSEKMTTEVFCRSRILLPKLRLESRYDHN |
| | | QMLLDMLSELSRCPKLLYEKLSEKDKKHFQVEADGFLDEIEEEQNPF |
| | | KDALIRHQDRFPYFALRYLDLNESFKSIRFQVDLGTYHYCIYDKKIGDE |
| | | QEKRHLTRTLLSFGRLQDFTEINRPQEWKALTKDLDYKETSKQPFISK |
| | | TTPHYHITDNKIGFRLGTSKELYPSLEVKDGANRIAKYPYNSDFVAHA |
| | | FISVHELLPLMFYQHLTGKSEDLLKETVRHIQRIYKDFEEERINTIEDLE |
| | | KANQGRLPLGAFPKQMLGLLQNKQPDLSEKAKIKIEKLIAETKLLSHR |
| | | LNTKLKSSPKLGKRREKLIKTGDWLVKDFMRFQPVAYDVQNQPIESS |
| | | KANSTEFQLIQRALALYGGEKNRLEGYFKQTNLIGNTNPHPFLNKFN |
| | | WKACRNLVDFYQQYLEQREKFLEAIKNQPWEPYQYCLLLKIPKENR |
| | | KNLVKGWEQGGISLPRGLFTEAIRETLSEDLTLSKPIRKEIKKHGRVGF |
| | | ISRAITLYFRERYQDDHQSFYNLPYELEAKASTPKPPLPKKREYVLRAE |
| | | HYEYWQQNKPQSPTELQRLELHTSDRWKDYLLYKRWQHLEKKLRL |
| | | YRNQDVMLWLMTLELTKNHFKELKLNYHQLKLENLAVNVQEADAK |
| | | LNPLNQTLPMVLPVKVYPATAFGEVQYQETPIRTVYIREEQTKALKM |
| | | GNFKALVKDRRLNGLFSFIKEENDTQKHPISQLRLRRELEIYQSLRVD |
| | | AFKETLSLEEKLLNKHASLSSLENEFRTLLEEWKKKYAASSMVTDEHI |
| | | AFIASVRNAFCHNQYPFYKETLHAPILLFTVAQPTTEEKDGLGIAEALL |
| | | RVLREYCEIVKSQI |
| 237 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQ.TNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 238 | Cas13 | MENKTSLGNNIYYNPFKPQDKSYFAGYFNAAMENTDSVFRELGKRL |
| | | KGKEYTSENFFDAIFKENISLVEYERYVKLLSDYFPMARLLDKKEVPIK |
| | | ERKENFKKNFKGIIKAVRDLRNFYTHKEHGEVEITDEIFGVLDEMLKS |
| | | TVLTVKKKKVKTDKTKEILKKSIEKQLDILCQKKLEYLRDTARKIEEKRR |
| | | NQRERGEKELVAPFKYSDKRDDLIAAIYNDAFDVYIDKKKDSLKESSK |
| | | AKYNTKSDPQQEEGDLKIPISKNGVVFLLSLFLTKQEIHAFKSKIAGFK |
| | | ATVIDEATVSEATVSHGKNSICFMATHEIFSHLAYKKLKRKVRTAEIN |
| | | YGEAENAEQLSVYAKETLMMQMLDELSKVPDVVYQNLSEDVQKTF |
| | | IEDW/NEYLKENNGDVGTMEEEQVIHPVIRKRYEDKFNYFAIRFLDEF |
| | | AQFPTLRFQVHLGNYLHDSRPKENLISDRRIKEKITVFGRLSELEHKKA |
| | | LFIKNTETNEDREHYWEIFPNPNYDFPKENISVNDKDFPIAGSILDRE |
| | | KQPVAGKIGIKVKLLNQQYVSEVDKAVKAHQLKQRKASKPSIQNIIEE |
| | | IVPINESNPKEAIVFGGQPTAYLSMNDIHSILYEFFDKWEKKKEKLEK |
| | | KGEKELRKEIGKELEKKIVGKIQAQIQQIIDKDTNAKILKPYQDGNSTA |
| | | IDKEKLIKDLKQEQNILQKLKDEQTVREKEYNDFIAYQDKNREINKVR |
| | | DRNHKQYLKDNLKRKYPEAPARKEVLYYREKGKVAVWLANDIKRF |
| | | MPTDFKNEWKGEQHSLLQKSLAYYEQCKEELKNLLPEKVFQHLPFK |
| | | LGGYFQQKYLYQFYTCYLDKRLEYISGLVQQAENFKSENKVFKKVEN |
| | | ECFKFLKKQNYTHKELDARVQSILGYPIFLERGFMDEKPTIIKGKTFKG |
| | | NEALFADWFRYYKEYQNFQTFYDTENYPLVELEKKQADRKRKTKIYQ |
| | | QKKNDVFTLLMAKHIFKSVFKQDSIDQFSLEDLYQSREERLGNQERA |
| | | RQTGERNTNYIWNKTVDLKLCDGKITVENVKLKNVGDFIKYEYDQR |
| | | VQAFLKYEENIEWQAFLIKESKEEENYPYVVEREIEQYEKVRREELLKE |
| | | VHLIEEYILEKVKDKEILKKGDNQNFKYYILNGLLKQLKNEDVESYKVF |
| | | NLNTEPEDVNINQLKQEATDLEQKAFVLTYIRNKFAHNQLPKKEFW |
| | | DYCQEKYGKIEKEKTYAEYFAEVFKKEKEALIK |
| 239 | Cas13 | MFFSFHNAQRVIFKHLYKAFDASLRMVKEDYKAHFTVNLTRDFAHL |
| | | NRKGKNKQDNPDFNRYRFEKDGFFTESGLLFFTNLFLDKRDAYWM |
| | | LKKVSGFKASHKQREKMTTEVFCRSRILLPKLRLESRYDHNQMLLD |
| | | MLSELSRCPKLLYEKLSEENKKHFQVEADGFLDEIEEEQNPFKDTLIR |
| | | HQDRFPYFALRYLDLNESFKSIRFQVDLGTYHYCIYDKKIGDEQEKRH |
| | | LTRTLLSFGRLQDFTEINRPQEWKALTKDLDYKETSNQPFISKTTPHY |
| | | HITDNKIGFRLGTSKELYPSLEIKDGANRIAKYPYNSGFVAHAFISVHE |
| | | LLPLMFYQHLTGKSEDLLKETVRHIQRIYKDFEEERINTIEDLEKANQG |
| | | RLPLGAFPKQMLGLLQNKQPDLSEKAKIKIEKLIAETKLLSHRLNTKLK |
| | | SSPKLGKRREKLIKTGVLADWLVKDFMRFQPVAYDAQNQPIKSSKA |
| | | NSTEFWFIRRALALYGGEKNRLEGYFKQTNLIGNTNPHPFLNKFNW |
| | | KACRNLVDFYQQYLEQREKFLEAIKNQPWEPYQYCLLLKIPKENRKN |
| | | LVKGWEQGGISLPRGLFTEAIRETLSEDLMLSKPIRKEIKKHGRVGFIS |
| | | RAITLYFKEKYQDKHQSFYNLSYKLEAKAPLLKREEHYEYWQQNKPQ |
| | | SPTESQRLELHTSDRWKDYLLYKRWQHLEKKLRLYRNQDVMLWLM |
| | | TLELTKNHFKELNLNYHQLKLENLAVNVQEADAKLNPLNQTLPMVL |
| | | PVKVYPATAFGEVQYHKTPIRTVYIREEHTKALKMGNFKALVKDRRL |
| | | NGLFSFIKEENDTQKHPISQLRLRRELEIYQSLRVDAFKETLSLEEKLLN |
| | | KHTSLSSLENEFRALLEEWKKEYAASSMVTDEHIAFIASVRNAFCHN |
| | | QYPFYKEALHAPIPLFTVAQPTTEEKDGLGIAEALLKVLREYCEIVKSQI |
| 240 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQ.TNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 241 | Cas13 | MEKPLPPNVYTLKHKFFWGAFLNIARHNAFITICHINEQLGLTTPPN |
| | | DDKIADVVCGTWNNILNNDHDLLKKSQLTELILKHFPFLAAMCYHP |
| | | PKKEGKKKGSQKEQQKEKENEAQSQAEALNPSELIKALKTLVKQLRT |
| | | LRNYYSHYKHKKPDAEKDIFKHLYKAFDASLRMVKEDYKAHFTVNLT |
| | | QDFAHLNRKGKNKQDNPKFDRYRFEKDGFFTESGLLFFTNLFLDKR |
| | | DAYWMLKKVSGFKASHKQSEKMTTEVFCRSRILLPKLRLESRYDHN |
| | | QMLLDMLSELSRCPKLLYEKLSEKDKKHFQVEADGFLDEIEEEQNPF |
| | | KDALIRHQDRFPYFALRYLDLNESFKSIRFQVDLGTYHYCIYDKKIGDE |
| | | QEKRHLTRTLLSFGRLQDFTEINRPQEWKALTKDLDYKETSKQPFISK |
| | | TTPHYHITDNKIGFRLGTSKELYPSLEVKDGANRIAKYPYNSDFVAHA |
| | | FISVHELLPLMFYQHLTGKSEDLLKETVRHIQRIYKDFEEERINTIEDLE |
| | | KANQGRLPLGAFPKQMLGLLQNKQPDLSEKAKIKIEKLIAETKLLSHR |
| | | LNTKLKSSPKLGKRREKLIKTGVLADWLVKDFMRFQPVAYDVQNQP |
| | | IESSKANSTEFQLIQRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLTLSKPIRKEIKKHGRV |
| | | GFISRAITLYFRERYQDDHQSFYNLPYELEAKASTPKPPLPKKREYVLR |
| | | AEHYEYWQQNKPQSPTELQRLELHTSDRWKDYLLYKRWQHLEKKL |
| | | RLYRNQDVMLWLMTLELTKNHFKELKLNYHQLKLENLAVNVQEAD |
| | | AKLNPLNQTLPMVLPVKVYPATAFGEVQYQETPIRTVYIREEQTKAL |
| | | KMGNFKALVKDRRLNGLFSFIKEENDTQKHPISQLRLRRELEIYQSLR |
| | | VDAFKETLSLEEKLLNKHASLSSLENEFRTLLEEWKKKYAASSMVTDE |
| | | HIAFIASVRNAFCHNQYPFYKETLHAPILLFTVAQPTTEEKDGLGIAEA |
| | | LLRVLREYCEIVKSQI |
| 242 | Cas13 | MEKPLLPNVYTLKHKFFWGAFLNIARHNAFITICHINEQLGLKTPSN |
| | | DDKIVDWCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 243 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 244 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 245 | Cas13 | MFFSFHNAQRVIFKHLYKAFDASLRMVKEDYKAHFTVNLTRDFAHL |
| | | NRKGKNKQDNPDFNRYRFEKDGFFTESGLLFFTNLFLDKRDAYWM |
| | | LKKVSGFKASHKQREKMTTEVFCRSRILLPKLRLESRYDHNQMLLD |
| | | MLSELSRCPKLLYEKLSEENKKHFQVEADGFLDEIEEEQNPFKDTLIR |
| | | HQDRFPYFALRYLDLNESFKSIRFQVDLGTYHYCIYDKKIGDEQEKRH |
| | | LTRTLLSFGRLQDFTEINRPQEWKALTKDLDYKETSNQPFISKTTPHY |
| | | HITDNKIGFRLGTSKELYPSLEIKDGANRIAKYPYNSGFVAHAFISVHE |
| | | LLPLMFYQHLTGKSEDLLKETVRHIQRIYKDFEEERINTIEDLEKANQG |
| | | RLPLGAFPKQMLGLLQNKQPDLSEKAKIKIEKLIAETKLLSHRLNTKLK |
| | | SSPKLGKRREKLIKTGVLADWLVKDFMRFQPVAYDAQNQPIKSSKA |
| | | NSTEFWFIRRALALYGGEKNRLEGYFKQ.TNLIGNTNPHPFLNKFNW |
| | | KACRNLVDFYQQYLEQREKFLEAIKNQPWEPYQYCLLLKIPKENRKN |
| | | LVKGWEQGGISLPRGLFTEAIRETLSEDLMLSKPIRKEIKKHGRVGFIS |
| | | RAITLYFKEKYQDKHQSFYNLSYKLEAKAPLLKREEHYEYWQQNKPQ |
| | | SPTESQRLELHTSDRWKDYLLYKRWQHLEKKLRLYRNQDVMLWLM |
| | | TLELTKNHFKELNLNYHQLKLENLAVNVQEADAKLNPLNQTLPMVL |
| | | PVKVYPATAFGEVQYHKTPIRTVYIREEHTKALKMGNFKALVKDRRL |
| | | NGLFSFIKEENDTQKHPISQLRLRRELEIYQSLRVDAFKETLSLEEKLLN |
| | | KHTSLSSLENEFRALLEEWKKEYAASSMVTDEHIAFIASVRNAFCHN |
| | | QYPFYKEALHAPIPLFTVAQPTTEEKDGLGIAEALLKVLREYCEIVKSQI |
| 246 | Cas13 | MDTPNFSERIPVSLQSHPYYFAHYLNMARHNAYVILEYVNRELIKPG |
| | | KNLDEDNLIQSTVLKDGYFDRKPDELSHRNRLLVQHFPFLREAENEG |
| | | ARTCNPVSYKLKTALAALNQWRNNASHYPLNQNHEKDFDLQPFFS |
| | | FAIEACKKRMREVFQPDDFYLLETNEKQFYTLHNENGFTEKGLYCFIC |
| | | FFLEKKYAFQFLAGIKGFKNTTDNKFRATLETFTEHCCRLPKPKLDSS |
| | | DIKLDMLGELSRCPAPLFDLLDIEERKKFIREPEEVKPDESGDREEVQ |
| | | QVLMKRYDDRFPYFALRYFEEKNLLKGISFHIHIGRWIKSEHTKKIMG |
| | | AERDRRLLKDIRTFGELKEFSPEHAPDYWLRDGITPDDVDQFSPQYR |
| | | IVGNRIGIKLNYNGHNRWSVPDKEINVKPDAIISTYEFLNLFLYEHLY |
| | | QKKLTGLSPAEFIQDYLDRFNNFLSEFKAGHIRPVGDFSLEKRRGQG |
| | | DEPDLTARRKSLQKELDRFVLKGKDLPDKIREYLLGYKQKSEKKQAK |
| | | WILGGMIKETVYWRNKAEQSPEKMRSGDMAQQLARDIIFLTPPHT |
| | | VKEHKQKLNSLEYDVLQYALAYFSSNREKLYSFFKEHQLTVKGDRAH |
| | | PFLYKIRLDECQGILDFFIVYMQQKEKWLGWLDRNLKSPRLNEEEFF |
| | | NTYSYFIKTDTKRAIEMDYESCPNYLPRGIFNEPIAKALQKAGVKIKDE |
| | | DNASYALSVYSNGKTQPFYNKERYYNKGIFRMEELPEKLQPKELLGKI |
| | | QWTIKSSGKDTEEFRSLQNLKNRILNTEKEIRYVQSTDRALWIMVAD |
| | | LFPETFELRPDDLECIGHDLSDDLLSRPYQMKEKVYNYTITDYLPIKRY |
| | | GEFRRFLKDRRLENLLTYFEEGVPLHREALVAELEAYDLQRKNLLEIIY |
| | | RFEKLVFDRHRHELTFSGEGENQYVNHWDYLDFVARKYGLSAEVKE |
| | | LNSERFTELRNKMLHNQIPYQLWIKEAIAAREENTVCGRIMGMIGEI |
| | | YERMTTEIEKQMQV |
| 247 | Cas13 | MTEQNEKPYNGTYYTLEDKHFWAAFLNLARHNAYITLTHIDRQLAY |
| | | SKADITNDEDILFFKGQWKNLDNDLERKARLRSLILKHFFFLEGAAYG |
| | | KKLFESQSSGNKSSKKKELTKKEKEELQANALSLDNLKSILFDFLQKLK |
| | | DFRNYYSHYRHPESSELPLFDGNMLQRLYNVFDVSVQRVKRDHEH |
| | | NDKVDPHRHFNHLVRKGKKDKYGNNDNPFFKHHFVDREGKVTEA |
| | | GLLFFVSLFLEKRDAIWMQKKIRGFKGGTETYQQMTNEVFCRSRISL |
| | | PKLKLESLRTDDWMLLDMLNELVRCPKSLYDRLREEDRARFRVPVDI |
| | | LSDEDDTDGAEEDPFKNTLVRHQDRFPYFALRYFDLKKVFTSLRFHID |
| | | LGTYHFAIYKKNIGEQPEDRHLTRNLYGFGRIQDFAEEHRPEEWKRL |
| | | VRDLDYFETGDKPYITQTTPHYHIEKGKIGLRFVPEGQHLWPSPEVG |
| | | ATRTGRSKYAQDKRFTAEAFLSVHELMPMMFYYFLLREKYSEEASAE |
| | | RVQGRIKRVIEDVYAVYDAFARDEINTRDELDACLADKGIRRGHLPR |
| | | QMIAILSQKHKDMEEKVRKKLQEMIADTDHRLDMLDRQTDRKIRIG |
| | | RKNAGLPKSGVIADWLVRDMMRFQPVAKDTSGKPLNNSKANSTE |
| | | YRMLQRALALFGGEKERLTPYFRQMNLTGGNNPHPFLHETRWESH |
| | | TNILSFYRSYLKARKAFLQSIGRSDRVENHRFLLLKEPKTDRQTLVAG |
| | | WKGEFHLPRGIFTEAVRDCLIEMGLDEVRSYKEVGFMAKAVPLYFE |
| | | RASKDRVQPFYDYPFNVGNSLKPKKGRFLSKEKRAEEWESGKERFR |
| | | DLEAWSHSAARRIEDAFAGIENASRENKKKIEQLLQDLSLWETFESKL |
| | | KVKADKINIAKLKKEILEAKEHPYLDFKSWQKFERELRLVKNQDIITW |
| | | MMCRDLMEENKVEGLDTGTLYLKDIRTDVQEQGSLNVLNHVKPM |
| | | RLPVVVYRADSRGHVHKEQAPLATVYIEERDTKLLKQGNFKSFVKDR |
| | | RLNGLFSFVDTGGLAMEQYPISKLRVEYELAKYQTARVCAFEQTLELE |
| | | ESLLTRYPHLPDKNFRKMLESWSDPLLAKWPELHEKVRLLIAVRNAF |
| | | SHNQYPMYDEAVFSPIRKYDPSSPDAIEERMRLNIAHRLSEEVKQAK |
| | | ETVERIIQA |
| 248 | Cas13 | MDTPNFSERIPVSLQSHPYYFAHYLNMARHNAYVILEYVNRELIKPG |
| | | KNLDEDNLIQSTVLKDGYFDRKPDELSHRNRLLVQHFPFLREAENEG |
| | | ARTCNPVSYKLKTALAALNQWRNNASHYPLNQNHEKDFDLQPFFS |
| | | FAIEACKKRMREVFQPDDFYLLETNEKQFYTLHNENGFTEKGLYCFIC |
| | | FFLEKKYAFQFLAGIKGFKNTTDNKFRATLETFTEHCCRLPKPKLDSS |
| | | DIKLDMLGELSRCPAPLFDLLDIEERKKFIREPEEVKPDESGDREEVQ |
| | | QVLMKRYDDRFPYFALRYFEEKNLLKGISFHIHIGRWIKSEHTKKIMG |
| | | AERDRRLLKDIRTFGELKEFSPEHAPDYWLRDGITPDDVDQFSPQYR |
| | | IVGNRIGIKLNYNGHNRWSVPDKEINVKPDAIISTYEFLNLFLYEHLY |
| | | QKKLTGLSPAEFIQDYLDRFNNFLSEFKAGHIRPVGDFSLEKRRGQG |
| | | DEPDLTARRKSLQKELDRFVLKGKDLPDKIREYLLGYKQKSEKKQAK |
| | | WILGGMIKETVYWRNKAEQSPEKMRSGDMAQQLARDIIFLTPPHT |
| | | VKEHKQKLNSLEYDVLQYALAYFSSNREKLYSFFKEHQLTVKGDRAH |
| | | PFLYKIRLDECQGILDFFIVYMQQKEKWLGWLDRNLKSPRLNEEEFF |
| | | NTYSYFIKTDTKRAIEMDYESCPNYLPRGIFNEPIAKALQKAGVKIKDE |
| | | DNASYALSVYSNGKTQPFYNKERYYNKGIFRMEELPEKLQPKELLGKI |
| | | QWTIKSSGKDTEEFRSLQNLKNRILNTEKEIRYVQSTDRALWIMVAD |
| | | LFPETFELRPDDLECIGHDLSDDLLSRPYQMKEKVYNYTITDYLPIKRY |
| | | GEFRRFLKDRRLENLLTYFEEGVPLHREALVAELEAYDLQRKNLLEIIY |
| | | RFEKLVFDRHRHELTFSGEGENQYVNHWDYLDFVARKYGLSAEVKE |
| | | LNSERFTELRNKMLHNQIPYQLWIKEAIAAREENTVCGRIMGMIGEI |
| | | YERMTTEIEKQMQV |
| 249 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSRIPIELYQTLEPKYREMYNATLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KQLIESPTREKSPALNVSYLILNYFRTYFEDQSQEFYAQPRNYRLFDKL |
| | | SPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRINSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIDSSFLIKTRNMFLHDKYEAECIKEIS |
| | | DDFVYAKKIIAEFKMKIENIKLEDFSNDSSA |
| 250 | Cas13 | MEKPLPPNVYTLKHKFFWGAFLNIARHNAFITICHINEQLGLTTPPN |
| | | DDKIADVVCGTWNNILNNDHDLLKKSQLTELILKHFPFLAAMCYHP |
| | | PKKEGKKKGSQKEQQKEKENEAQSQAEALNPSELIKALKTLVKQLRT |
| | | LRNYYSHYKHKKPDAEKDIFKHLYKAFDASLRMVKEDYKAHFTVNLT |
| | | QDFAHLNRKGKNKQDNPKFDRYRFEKDGFFTESGLLFFTNLFLDKR |
| | | DAYWMLKKVSGFKASHKQSEKMTTEVFCRSRILLPKLRLESRYDHN |
| | | QMLLDMLSELSRCPKLLYEKLSEKDKKHFQVEADGFLDEIEEEQNPF |
| | | KDALIRHQDRFPYFALRYLDLNESFKSIRFQVDLGTYHYCIYDKKIGDE |
| | | QEKRHLTRTLLSFGRLQDFTEINRPQEWKALTKDLDYKETSKQPFISK |
| | | TTPHYHITDNKIGFRLGTSKELYPSLEVKDGANRIAKYPYNSDFVAHA |
| | | FISVHELLPLMFYQHLTGKSEDLLKETVRHIQRIYKDFEEERINTIEDLE |
| | | KANQGRLPLGAFPKQMLGLLQNKQPDLSEKAKIKIEKLIAETKLLSHR |
| | | LNTKLKSSPKLGKRREKLIKTGVLADWLVKDFMRFQPVAYDVQNQP |
| | | IESSKANSTEFQLIQRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLTLSKPIRKEIKKHGRV |
| | | GFISRAITLYFRERYQDDHQSFYNLPYELEAKASTPKPPLPKKREYVLR |
| | | AEHYEYWQQNKPQSPTELQRLELHTSDRWKDYLLYKRWQHLEKKL |
| | | RLYRNQDVMLWLMTLELTKNHFKELKLNYHQLKLENLAVNVQEAD |
| | | AKLNPLNQTLPMVLPVKVYPATAFGEVQYQETPIRTVYIREEQTKAL |
| | | KMGNFKALVKDRRLNGLFSFIKEENDTQKHPISQLRLRRELEIYQSLR |
| | | VDAFKETLSLEEKLLNKHASLSSLENEFRTLLEEWKKKYAASSMVTDE |
| | | HIAFIASVRNAFCHNQYPFYKETLHAPILLFTVAQPTTEEKDGLGIAEA |
| | | LLRVLREYCEIVKSQI |
| 251 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 252 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQ.TNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 253 | Cas13 | MFFSFHNAQRVIFKHLYKAFDASLRMVKEDYKAHFTVNLTRDFAHL |
| | | NRKGKNKQDNPDFNRYRFEKDGFFTESGLLFFTNLFLDKRDAYWM |
| | | LKKVSGFKASHKQREKMTTEVFCRSRILLPKLRLESRYDHNQMLLD |
| | | MLSELSRCPKLLYEKLSEENKKHFQVEADGFLDEIEEEQNPFKDTLIR |
| | | HQDRFPYFALRYLDLNESFKSIRFQVDLGTYHYCIYDKKIGDEQEKRH |
| | | LTRTLLSFGRLQDFTEINRPQEWKALTKDLDYKETSNQPFISKTTPHY |
| | | HITDNKIGFRLGTSKELYPSLEIKDGANRIAKYPYNSGFVAHAFISVHE |
| | | LLPLMFYQHLTGKSEDLLKETVRHIQRIYKDFEEERINTIEDLEKANQG |
| | | RLPLGAFPKQMLGLLQNKQPDLSEKAKIKIEKLIAETKLLSHRLNTKLK |
| | | SSPKLGKRREKLIKTGVLADWLVKDFMRFQPVAYDAQNQPIKSSKA |
| | | NSTEFWFIRRALALYGGEKNRLEGYFKQ.TNLIGNTNPHPFLNKFNW |
| | | KACRNLVDFYQQYLEQREKFLEAIKNQPWEPYQYCLLLKIPKENRKN |
| | | LVKGWEQGGISLPRGLFTEAIRETLSEDLMLSKPIRKEIKKHGRVGFIS |
| | | RAITLYFKEKYQDKHQSFYNLSYKLEAKAPLLKREEHYEYWQQNKPQ |
| | | SPTESQRLELHTSDRWKDYLLYKRWQHLEKKLRLYRNQDVMLWLM |
| | | TLELTKNHFKELNLNYHQLKLENLAVNVQEADAKLNPLNQTLPMVL |
| | | PVKVYPATAFGEVQYHKTPIRTVYIREEHTKALKMGNFKALVKDRRL |
| | | NGLFSFIKEENDTQKHPISQLRLRRELEIYQSLRVDAFKETLSLEEKLLN |
| | | KHTSLSSLENEFRALLEEWKKEYAASSMVTDEHIAFIASVRNAFCHN |
| | | QYPFYKEALHAPIPLFTVAQPTTEEKDGLGIAEALLKVLREYCEIVKSQI |
| 254 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 255 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQ.TNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 256 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 257 | Cas13 | MDTPNFSERIPVSLQSHPYYFAHYLNMARHNAYVILEYVNRELIKPG |
| | | KNLDEDNLIQSTVLKDGYFDRKPDELSHRNRLLVQHFPFLREAENEG |
| | | ARTCNPVSYKLKTALAALNQWRNNASHYPLNQNHEKDFDLQPFFS |
| | | FAIEACKKRMREVFQPDDFYLLETNEKQFYTLHNENGFTEKGLYCFIC |
| | | FFLEKKYAFQFLAGIKGFKNTTDNKFRATLETFTEHCCRLPKPKLDSS |
| | | DIKLDMLGELSRCPAPLFDLLDIEERKKFIREPEEVKPDESGDREEVQ |
| | | QVLMKRYDDRFPYFALRYFEEKNLLKGISFHIHIGRWIKSEHTKKIMG |
| | | AERDRRLLKDIRTFGELKEFSPEHAPDYWLRDGITPDDVDQFSPQYR |
| | | IVGNRIGIKLNYNGHNRWSVPDKEINVKPDAIISTYEFLNLFLYEHLY |
| | | QKKLTGLSPAEFIQDYLDRFNNFLSEFKAGHIRPVGDFSLEKRRGQG |
| | | DEPDLTARRKSLQKELDRFVLKGKDLPDKIREYLLGYKQKSEKKQAK |
| | | WILGGMIKETVYWRNKAEQSPEKMRSGDMAQQLARDIIFLTPPHT |
| | | VKEHKQKLNSLEYDVLQYALAYFSSNREKLYSFFKEHQLTVKGDRAH |
| | | PFLYKIRLDECQGILDFFIVYMQQKEKWLGWLDRNLKSPRLNEEEFF |
| | | NTYSYFIKTDTKRAIEMDYESCPNYLPRGIFNEPIAKALQKAGVKIKDE |
| | | DNASYALSVYSNGKTQPFYNKERYYNKGIFRMEELPEKLQPKELLGKI |
| | | QWTIKSSGKDTEEFRSLQNLKNRILNTEKEIRYVQSTDRALWIMVAD |
| | | LFPETFELRPDDLECIGHDLSDDLLSRPYQMKEKVYNYTITDYLPIKRY |
| | | GEFRRFLKDRRLENLLTYFEEGVPLHREALVAELEAYDLQRKNLLEIIY |
| | | RFEKLVFDRHRHELTFSGEGENQYVNHWDYLDFVARKYGLSAEVKE |
| | | LNSERFTELRNKMLHNQIPYQLWIKEAIAAREENTVCGRIMGMIGEI |
| | | YERMTTEIEKQMQV |
| 258 | Cas13 | MFFSFHNAQRVIFKHLYKAFDASLRMVKEDYKAHFTVNLTRDFAHL |
| | | NRKGKNKQDNPDFNRYRFEKDGFFTESGLLFFTNLFLDKRDAYWM |
| | | LKKVSGFKASHKQREKMTTEVFCRSRILLPKLRLESRYDHNQMLLD |
| | | MLSELSRCPKLLYEKLSEENKKHFQVEADGFLDEIEEEQNPFKDTLIR |
| | | HQDRFPYFALRYLDLNESFKSIRFQVDLGTYHYCIYDKKIGDEQEKRH |
| | | LTRTLLSFGRLQDFTEINRPQEWKALTKDLDYKETSNQPFISKTTPHY |
| | | HITDNKIGFRLGTSKELYPSLEIKDGANRIAKYPYNSGFVAHAFISVHE |
| | | LLPLMFYQHLTGKSEDLLKETVRHIQRIYKDFEEERINTIEDLEKANQG |
| | | RLPLGAFPKQMLGLLQNKQPDLSEKAKIKIEKLIAETKLLSHRLNTKLK |
| | | SSPKLGKRREKLIKTGVLADWLVKDFMRFQPVAYDAQNQPIKSSKA |
| | | NSTEFWFIRRALALYGGEKNRLEGYFKQTNLIGNTNPHPFLNKFNW |
| | | KACRNLVDFYQQYLEQREKFLEAIKNQPWEPYQYCLLLKIPKENRKN |
| | | LVKGWEQGGISLPRGLFTEAIRETLSEDLMLSKPIRKEIKKHGRVGFIS |
| | | RAITLYFKEKYQDKHQSFYNLSYKLEAKAPLLKREEHYEYWQQNKPQ |
| | | SPTESQRLELHTSDRWKDYLLYKRWQHLEKKLRLYRNQDVMLWLM |
| | | TLELTKNHFKELNLNYHQLKLENLAVNVQEADAKLNPLNQTLPMVL |
| | | PVKVYPATAFGEVQYHKTPIRTVYIREEHTKALKMGNFKALVKDRRL |
| | | NGLFSFIKEENDTQKHPISQLRLRRELEIYQSLRVDAFKETLSLEEKLLN |
| | | KHTSLSSLENEFRALLEEWKKEYAASSMVTDEHIAFIASVRNAFCHN |
| | | QYPFYKEALHAPIPLFTVAQPTTEEKDGLGIAEALLKVLREYCEIVKSQI |
| 259 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ. |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 260 | Cas13 | MTNTPKRRTLHRHPSYFGAFLNIARHNAFMIMEHLSTKYDMEDKN |
| | | TLDEAQLPNAKLFGCLKKRYGKPDVTEGVSRDLRRYFPFLNYPLFLHL |
| | | EKQQNAEQAATYDINPEDIEFTLKGFFRLLNQMRNNYSHYISNTDY |
| | | GKFDKLPVQDIYEAAIFRLLDRGKHTKRFDVFESKHTRHLESNNSEYR |
| | | PRSLANSPDHENTVAFVTCLFLERKYAFPFLSRLDCFRSTNDAAEGD |
| | | PLIRKASHECYTMFCCRLPQPKLESSDILLDMVNELGRCPSALYNLLS |
| | | EEDQARFHIKREEITGFEEDPDEELEQEIVLKRHSDRFPYFALRYFDDT |
| | | EAFQTLRFDVYLGRWRTKPVYKKRIYGQERDRVLTQSIRTFTRLSRLL |
| | | PIYENVKHDAVRQNEEDGKLVNPDVTSQFHKSWIQIESDDRAFLSD |
| | | RIEHFSPHYNFGDQVIGLKFINPDRYAAIQNVFPKLPGEEKKDKDAKL |
| | | VNETADAIISTHEIRSLFLYHYLSKKPISAGDERRFIQVDTETFIKQYIDT |
| | | IKLFFEDIKSGELQPIADPPNYQKNEPLPYVRGDKEKTQEERAQYRER |
| | | QKEIKERRKELNTLLQNRYGLSIQYIPSRLREYLLGYKKVPYEKLALQKL |
| | | RAQRKEVKKRIKDIEKMRTPRVGEQATWLAEDIVFLTPPKMHTPER |
| | | KTTKHPQKLNNDQFRIMQSSLAYFSVNKKAIKKFFQKETGIGLSNRE |
| | | TSHPFLYRIDVGRCRGILDFYTGYLKYKMDWLDDAIKKVDNRKHGK |
| | | KEAKKYEKYLPSSIQHKTPLELDYTRLPVYLPRGLFKKAIVKALAAHAD |
| | | FQVEPEEDNVIFCLDQLLDGDTQDFYNWQRYYRSALTEKETDNQLV |
| | | LAHPYAEQILGTIKTLEGKQKNNKLGNKAKQKIKDELIDLKRAKRRLL |
| | | DREQYLRAVQAEDRALWLMIQERQKQKAEHEEIAFDQLDLKNITKI |
| | | LTESIDARLRIPDTKVDITDKLPLRRYGDLRRVAKDRRLVNLASYYHV |
| | | AGLSEIPYDLVKKELEEYDRRRVAFFEHVYQFEKEVYDRYAAELRNEN |
| | | PKGESTYFSHWEYVAVAVKHSADTHFNELFKEKVMQLRNKFHHNE |
| | | FPYFDWLLPEVEKASAALYADRVFDVAEGYYQKMRKLMRQ |
| 261 | Cas13 | MTNTPKRRTLHRHPSYFGAFLNIARHNAFMIMEHLSTKYDMEDKN |
| | | TLDEAQLPNAKLFGCLKKRYGKPDVTEGVSRDLRRYFPFLNYPLFLHL |
| | | EKQQNAEQAATYDINPEDIEFTLKGFFRLLNQMRNNYSHYISNTDY |
| | | GKFDKLPVQDIYEAAIFRLLDRGKHTKRFDVFESKHTRHLESNNSEYR |
| | | PRSLANSPDHENTVAFVTCLFLERKYAFPFLSRLDCFRSTNDAAEGD |
| | | PLIRKASHECYTMFCCRLPQPKLESSDILLDMVNELGRCPSALYNLLS |
| | | EEDQARFHIKREEITGFEEDPDEELEQEIVLKRHSDRFPYFALRYFDDT |
| | | EAFQTLRFDVYLGRWRTKPVYKKRIYGQERDRVLTQSIRTFTRLSRLL |
| | | PIYENVKHDAVRQNEEDGKLVNPDVTSQFHKSWIQIESDDRAFLSD |
| | | RIEHFSPHYNFGDQVIGLKFINPDRYAAIQNVFPKLPGEEKKDKDAKL |
| | | VNETADAIISTHEIRSLFLYHYLSKKPISAGDERRFIQVDTETFIKQYIDT |
| | | IKLFFEDIKSGELQPIADPPNYQKNEPLPYVRGDKEKTQEERAQYRER |
| | | QKEIKERRKELNTLLQNRYGLSIQYIPSRLREYLLGYKKVPYEKLALQKL |
| | | RAQRKEVKKRIKDIEKMRTPRVGEQATWLAEDIVFLTPPKMHTPER |
| | | KTTKHPQKLNNDQFRIMQSSLAYFSVNKKAIKKFFQKETGIGLSNRE |
| | | TSHPFLYRIDVGRCRGILDFYTGYLKYKMDWLDDAIKKVDNRKHGK |
| | | KEAKKYEKYLPSSIQHKTPLELDYTRLPVYLPRGLFKKAIVKALAAHAD |
| | | FQVEPEEDNVIFCLDQLLDGDTQDFYNWQRYYRSALTEKETDNQLV |
| | | LAHPYAEQILGTIKTLEGKQKNNKLGNKAKQKIKDELIDLKRAKRRLL |
| | | DREQYLRAVQAEDRALWLMIQERQKQKAEHEEIAFDQLDLKNITKI |
| | | LTESIDARLRIPDTKVDITDKLPLRRYGDLRRVAKDRRLVNLASYYHV |
| | | AGLSEIPYDLVKKELEEYDRRRVAFFEHVYQFEKEVYDRYAAELRNEN |
| | | PKGESTYFSHWEYVAVAVKHSADTHFNELFKEKVMQLRNKFHHNE |
| | | FPYFDWLLPEVEKASAALYADRVFDVAEGYYQKMRKLMRQ |
| 262 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQ.TNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 263 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQ.TNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 264 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 265 | Cas13 | MESIKNSQKSTGKTLQKDPPYFGLYLNMALLNVRKVENHIRKWLGD |
| | | VALLPEKSGFHSLLTTDNLSSAKWTRFYYKSRKFLPFLEMFDSDKKSY |
| | | ENRRETTECLDTIDRQKISSLLKEVYGKLQDIRNAFSHYHIDDQSVKH |
| | | TALIISSEMHRFIENAYSFALQKTRARFTGVFVETDFLQAEEKGDNKK |
| | | FFAIGGNEGIKLKDNALIFLICLFLDREEAFKFLSRATGFKSTKEKGFLA |
| | | VRETFCALCCRQPHERLLSVNPREALLMDMLNELNRCPDILFEMLD |
| | | EKDQKSFLPLLGEEEQAHILENSLNDELCEAIDDPFEMIASLSKRVRYK |
| | | NRFPYLMLRYIEEKNLLPFIRFRIDLGCLELASYPKKMGEENNYERSVT |
| | | DHAMAFGRLTDFHNEDAVLQQITKGITDEVRFSLYAPRYAIYNNKIG |
| | | FVRTGGSDKISFPTLKKKGGEGHCVAYTLQNTKSFGFISIYDLRKILLLS |
| | | FLDKDKAKNIVSGLLEQCEKHWKDLSENLFDAIRTELQKEFPVPLIRY |
| | | TLPRSKGGKLVSSKLADKQEKYESEFERRKEKLTEILSEKDFDLSQIPRR |
| | | MIDEWLNVLPTSREKKLKGYVETLKLDCRERLRVFEKREKGEHPVPP |
| | | RIGEMATDLAKDIIRMVIDQGVKQRITSAYYSEIQRCLAQYAGDDNR |
| | | RHLDSIIRELRLKDTKNGHPFLGKVLRPGLGHTEKLYQRYFEEKKEWL |
| | | EATFYPAASPKRVPRFVNPPTGKQKELPLIIRNLMKERPEWRDWKQ. |
| | | RKNSHPIDLPSQLFENEICRLLKDKIGKEPSGKLKWNEMFKLYWDKE |
| | | FPNGMQRFYRCKRRVEVFDKVVEYEYSEEGGNYKKYYEALIDEVVR |
| | | QKISSSKEKSKLQVEDLTLSVRRVFKRAINEKEYQLRLLCEDDRLLFMA |
| | | VRDLYDWKEAQLDLDKIDNMLGEPVSVSQVIQLEGGQPDAVIKAE |
| | | CKLKDVSKLMRYCYDGRVKGLMPYFANHEATQEQVEMELRHYED |
| | | HRRRVFNWVFALEKSVLKNEKLRRFYEESQGGCEHRRCIDALRKASL |
| | | VSEEEYEFLVHIRNKSAHNQFPDLEIGKLPPNVTSGFCECIWSKYKAII |
| | | CRIIPFIDPERRFFGKLLEQK |
| 266 | Cas13 | MEKPLPPNVYTLKHKFFWGAFLNIARHNAFITICHINEQLGLTTPPN |
| | | DDKIADVVCGTWNNILNNDHDLLKKSQLTELILKHFPFLAAMCYHP |
| | | PKKEGKKKGSQKEQQKEKEKEKENEAQSQAEALNPSELIKVLKTIVK |
| | | QLRTLRNYYSHHSHKKPDTEKDIFKHLYKAFDASLRMVKEDYKAHFT |
| | | VNLTRDFAHLNRKGKNKQDNPDFNRYRFEKDGFFTESGLLFFTNLFL |
| | | DKRDAYWMLKKVSGFKASHKQREKMTTEVFCRSHILLPKLRLESRY |
| | | DHNQMLLDMLSELSRCPKLLYEKLSEENKKHFQVEADGFLDEIEEEQ |
| | | NPFKDTLIRHQDRFPYFALRYLDLNESFKSIRFQVDLGTYHYCIYDKKI |
| | | GDEQEKRHLTRTLLSFGRLQDFTEINRPQEWKALTKDLDYKETSNQP |
| | | FISKTTPHYHITDNKIGFRLGTSKELYPSLEIKDGANRIAKYPYNSGFVA |
| | | HAFISVHELLPLMFYQHLTGKSEDLLKETVRHIQRIYKDFEEERINTIE |
| | | DLEKANQGRLPLGAFPKQMLGLLQNKQPDLSEKAKIKIEKLIAETKLL |
| | | SHRLNTKLKSSPKLGKRREKLIKTGVLADWLVKDFMRFQPVAYDAQ |
| | | NQPIKSSKANSTEFWFIRRALALYGGEKNRLEGYFKQTNLIGNTNPH |
| | | PFLNKFNWKACRNLVDFYQQYLEQREKFLEAIKNQPWEPYQYCLLL |
| | | KIPKENRKNLVKGWEQGGISLPRGLFTEAIRETLSEDLMLSKPIRKEIK |
| | | KHGRVGFISRAITLYFKEKYQDKHQSFYNLSYKLEAKAPLLKREEHYEY |
| | | WQQNKPQSPTESQRLELHTSDRWKDYLLYKRWQHLEKKLRLYRNQ |
| | | DIMLWLMTLELTKNHFKELNLNYHQLKLENLAVNVQEADAKLNPL |
| | | NQTLPMVLPVKVYPATAFGEVQYQETPIRTVYIREEHTKALKMGNF |
| | | KALVKDRRLNGLFSFIKEENDTQKHPISQLRLRRELEIYQSLRVDAFKE |
| | | TLDLEEKLLKKHTSLSSLENKFRILLEEWKKEYAASSMVTDEHIAFIAS |
| | | VRNAFCHNQYPFYEEALHAPIPLFTVAQQTTEEKDGLGIAEALLRVL |
| | | REYCEIVKSQI |
| 267 | Cas13 | MFFSFHNAQRVIFKHLYKAFDASLRMVKEDYKAHFTVNLTRDFAHL |
| | | NRKGKNKQDNPDFNRYRFEKDGFFTESGLLFFTNLFLDKRDAYWM |
| | | LKKVSGFKASHKQREKMTTEVFCRSRILLPKLRLESRYDHNQMLLD |
| | | MLSELSRCPKLLYEKLSEENKKHFQVEADGFLDEIEEEQNPFKDTLIR |
| | | HQDRFPYFALRYLDLNESFKSIRFQVDLGTYHYCIYDKKIGDEQEKRH |
| | | LTRTLLSFGRLQDFTEINRPQEWKALTKDLDYKETSNQPFISKTTPHY |
| | | HITDNKIGFRLGTSKELYPSLEIKDGANRIAKYPYNSGFVAHAFISVHE |
| | | LLPLMFYQHLTGKSEDLLKETVRHIQRIYKDFEEERINTIEDLEKANQG |
| | | RLPLGAFPKQMLGLLQNKQPDLSEKAKIKIEKLIAETKLLSHRLNTKLK |
| | | SSPKLGKRREKLIKTGVLADWLVKDFMRFQPVAYDAQNQPIKSSKA |
| | | NSTEFWFIRRALALYGGEKNRLEGYFKQTNLIGNTNPHPFLNKFNW |
| | | KACRNLVDFYQQYLEQREKFLEAIKNQPWEPYQYCLLLKIPKENRKN |
| | | LVKGWEQGGISLPRGLFTEAIRETLSEDLMLSKPIRKEIKKHGRVGFIS |
| | | RAITLYFKEKYQDKHQSFYNLSYKLEAKAPLLKREEHYEYWQQNKPQ |
| | | SPTESQRLELHTSDRWKDYLLYKRWQHLEKKLRLYRNQDVMLWLM |
| | | TLELTKNHFKELNLNYHQLKLENLAVNVQEADAKLNPLNQTLPMVL |
| | | PVKVYPATAFGEVQYHKTPIRTVYIREEHTKALKMGNFKALVKDRRL |
| | | NGLFSFIKEENDTQKHPISQLRLRRELEIYQSLRVDAFKETLSLEEKLLN |
| | | KHTSLSSLENEFRALLEEWKKEYAASSMVTDEHIAFIASVRNAFCHN |
| | | QYPFYKEALHAPIPLFTVAQPTTEEKDGLGIAEALLKVLREYCEIVKSQI |
| 268 | Cas13 | MEKPLPPNVYTLKHKFFWGAFLNIARHNAFITICHINEQLGLTTPPN |
| | | DDKIADVVCGTWNNILNNDHDLLKKSQLTELILKHFPFLAAMCYHP |
| | | PKKEGKKKGSQKEQQKEKEKEKENEAQSQAEALNPSELIKVLKTIVK |
| | | QLRTLRNYYSHHSHKKPDTEKDIFKHLYKAFDASLRMVKEDYKAHFT |
| | | VNLTRDFAHLNRKGKNKQDNPDFNRYRFEKDGFFTESGLLFFTNLFL |
| | | DKRDAYWMLKKVSGFKASHKQREKMTTEVFCRSHILLPKLRLESRY |
| | | DHNQMLLDMLSELSRCPKLLYEKLSEENKKHFQVEADGFLDEIEEEQ |
| | | NPFKDTLIRHQDRFPYFALRYLDLNESFKSIRFQVDLGTYHYCIYDKKI |
| | | GDEQEKRHLTRTLLSFGRLQDFTEINRPQEWKALTKDLDYKETSNQP |
| | | FISKTTPHYHITDNKIGFRLGTSKELYPSLEIKDGANRIAKYPYNSGFVA |
| | | HAFISVHELLPLMFYQHLTGKSEDLLKETVRHIQRIYKDFEEERINTIE |
| | | DLEKANQGRLPLGAFPKQMLGLLQNKQPDLSEKAKIKIEKLIAETKLL |
| | | SHRLNTKLKSSPKLGKRREKLIKTGVLADWLVKDFMRFQPVAYDAQ |
| | | NQPIKSSKANSTEFWFIRRALALYGGEKNRLEGYFKQTNLIGNTNPH |
| | | PFLNKFNWKACRNLVDFYQQYLEQREKFLEAIKNQPWEPYQYCLLL |
| | | KIPKENRKNLVKGWEQGGISLPRGLFTEAIRETLSEDLMLSKPIRKEIK |
| | | KHGRVGFISRAITLYFKEKYQDKHQSFYNLSYKLEAKAPLLKREEHYEY |
| | | WQQNKPQSPTESQRLELHTSDRWKDYLLYKRWQHLEKKLRLYRNQ |
| | | DIMLWLMTLELTKNHFKELNLNYHQLKLENLAVNVQEADAKLNPL |
| | | NQTLPMVLPVKVYPATAFGEVQYQETPIRTVYIREEHTKALKMGNF |
| | | KALVKDRRLNGLFSFIKEENDTQKHPISQLRLRRELEIYQSLRVDAFKE |
| | | TLDLEEKLLKKHTSLSSLENKFRILLEEWKKEYAASSMVTDEHIAFIAS |
| | | VRNAFCHNQYPFYEEALHAPIPLFTVAQQTTEEKDGLGIAEALLRVL |
| | | REYCEIVKSQI |
| 269 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEIEHLRRYTRKKGRVVLKTEDDHFYYTLVNNNDLSEKGYA |
| | | FFISMFLERKYSYLFLKKLSGFKRGDSLQYRLTLEVFTALSTKPPVERLR |
| | | TTKDTKQDRALDILNELSRIPIELYQTLEPKYREMYNETLQPTDAEDP |
| | | YGLPDRSRIRFRSRFEAFALHFLDKQADFKEIGFYTYLGNYFHNGYQK |
| | | TRVDRETKDRYINFQLAGFCKNIQDISAKKLSEALNVKSIDISTDSIPDI |
| | | NSFEPYLVQSTPHYIVNGNNIGIKVLPEGKDTYPTIDEKGAKMPIADF |
| | | WLSKYELPAMLFYTYLRNNNIHKSHCPLSVKDIIERSIHKSTKQKHPE |
| | | ERSELMLRRVMKAIFWTDSKLNEVERIKSQKSAFGKRQHEILKAGRI |
| | | AETLVRDMLWLQPSKNNGRDKVTEPNFQAIQVSLAYFGIRRNDLTE |
| | | IFTRAGLINSSNPHPFLAQIGTNYTSLIEFYIAYLKERKVYFSRIQKKILQ |
| | | GKLNIQCHPLRDLQREPNKPQDKEEAIFLPRGLFNEAIINCLKKSKLK |
| | | QLIESPTREKSPALNVSYLIQNYFRTYFEDQSQEFYAQPRNYRLFDKL |
| | | SPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDLSNDSSA |
| 270 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKDWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 271 | Cas13 | MEKPLPPNVYTLKHKFFWGAFLNIARHNAFITICHINEQLGLTTPPN |
| | | DDKIADVVCGTWNNILNNDHDLLKKSQLTELILKHFPFLAAMCYHP |
| | | PKKEGKKKGSQKEQQKEKENEAQSQAEALNPSELIKALKTLVKQLRT |
| | | LRNYYSHYKHKKPDAEKDIFKHLYKAFDASLRMVKEDYKAHFTVNLT |
| | | QDFAHLNRKGKNKQDNPKFDRYRFEKDGFFTESGLLFFTNLFLDKR |
| | | DAYWMLKKVSGFKASHKQSEKMTTEVFCRSRILLPKLRLESRYDHN |
| | | QMLLDMLSELSRCPKLLYEKLSEKDKKHFQVEADGFLDEIEEEQNPF |
| | | KDALIRHQDRFPYFALRYLDLNESFKSIRFQVDLGTYHYCIYDKKIGDE |
| | | QEKRHLTRTLLSFGRLQDFTEINRPQEWKALTKDLDYKETSKQPFISK |
| | | TTPHYHITDNKIGFRLGTSKELYPSLEVKDGANRIAKYPYNSDFVAHA |
| | | FISVHELLPLMFYQHLTGKSEDLLKETVRHIQRIYKDFEEERINTIEDLE |
| | | KANQGRLPLGAFPKQMLGLLQNKQPDLSEKAKIKIEKLIAETKLLSHR |
| | | LNTKLKSSPKLGKRREKLIKTGVLADWLVKDFMRFQPVAYDVQNQP |
| | | IESSKANSTEFQLIQRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLTLSKPIRKEIKKHGRV |
| | | GFISRAITLYFRERYQDDHQSFYNLPYELEAKASTPKPPLPKKREYVLR |
| | | AEHYEYWQQNKPQSPTELQRLELHTSDRWKDYLLYKRWQHLEKKL |
| | | RLYRNQDVMLWLMTLELTKNHFKELKLNYHQLKLENLAVNVQEAD |
| | | AKLNPLNQTLPMVLPVKVYPATAFGEVQYQETPIRTVYIREEQTKAL |
| | | KMGNFKALVKDRRLNGLFSFIKEENDTQKHPISQLRLRRELEIYQSLR |
| | | VDAFKETLSLEEKLLNKHASLSSLENEFRTLLEEWKKKYAASSMVTDE |
| | | HIAFIASVRNAFCHNQYPFYKETLHAPILLFTVAQPTTEEKDGLGIAEA |
| | | LLRVLREYCEIVKSQI |
| 272 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 273 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQ.TNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 274 | Cas12a | MKSLAQFQNLYALQKTLRFELKPEGHTRETFNRWLEEIEKEQASENE |
| | | NIVYQDLLRAKKYEKIKIILDEYHKDFIEQALAYANLTELEKYEELYRKS |
| | | NRTSEEEEEFENTKESLRKQIANIFIKNPNKTVQERWKFLFSKKLIQNE |
| | | LIVWVKGNYELLSEKLKNEFPDESSIISTIEDFKYFTTYFRNYHENRKNL |
| | | YSNEDKFSTIAHRLIHENLPKFIDNIAIYQKAKAVLNINEVEKELGLPED |
| | | TLDKIFSLDFFSKALTQKGIDQYNYFLGGKTENEVKKIKGLNEFINLYN |
| | | QQQQDKNQRLPFLKVLYKLPLFERTSTSFRFEPIENDRDLIERIGKFYY |
| | | NDLKQYRDDSQGDTTDILSGINTLLRHVHDYRDGLYVNGGITLTQIS |
| | | QKIFGSWSYINNALAYFYDTYIDASGVDHQGERKPKKQKQIQEKTK |
| | | WLKQKQFPVILVEKALSEYKSIETNEDLKTRISDTTLCDFFKRCGNDD |
| | | NGQDLFDRIEARLREKNEEGYSLEDLLKKEFTTERKLMQDKTKTLLIK |
| | | NFLDVIQGDKDDITAGLLHFVKCLIPRTEISEKNELFYSGMEKYLNILS |
| | | EVTPLYNKARNYLTQKPYSIEKVKLNFENSTLLDGWDENEESDNSCV |
| | | LLRKRGYYYLGIMNKKHNMIFDRKIYPKATEGEAYYEKMIYKLLPGAY |
| | | KMLPKVFFSEKNIDYFKPSEEILRIRNTASYSKNGQPQEGYQKASFSIE |
| | | DCRKYIDFFKKCIANHWDWQKFNFNFSPTEYYQSIDEFYREIERQGY |
| | | KIDFVKIPESYINQLIKENKLYLFKIYNKDFSEKKKSKGKDNLHTLYWK |
| | | MLFDEKNLKDVVLKLNGEAEVFFRQKSILYNEEIWNKGHHYSELKDR |
| | | FSYPIISNKRYAEDKFFLHVPITLNFKADGINNVNNMVNEFIKDNRDI |
| | | HIIGIDRGERHLLYVSVINQKGDIVEQCSLNEIVTEYNGKIFKKNYHEE |
| | | LDNLEKERDRARKDWQTIANIKELKEGYLSHVIHKISKLILKYNAIVV |
| | | MEDLNSGFKRGRQKVEKQVYQNFEKQLIEKLNYLVLKESNVDEPGG |
| | | VLRAYQLANKFETFKKLGKQSGIIFYVPAAYTSAIDPVTGYIQYLYPLK |
| | | QADSVEKARKFYSQFKRISYNPHKQWFEFSFDYNDFNIIYHGKSSWT |
| | | ICTTNTERYMWNRLLNNGHGGEELVYVTNELELLFGEYNIIYGDGKD |
| | | IKQQITDVQDIDVDRTAKQFYKRINELLNLTLKLRHNNGKKGADEED |
| | | YILSPVEPYFDSRFESRKPSMQQTLPINADANGAFNIARKGLLLLERL |
| | | NQLGVEEFEKTKKSNNKKTQWLPHELWVEYAQNHTRK |
| 275 | Cas12a | MQDKTGWSSFTNKYSLSKTLRFELKPVGNTQKMLEDDGVFQKDRE |
| | | RQENYKKVKPFMDKLHREFIKEALNNLKLEGLTEYFEIFKKFRKDKNN |
| | | KELKNAEKKLRQIIGRCYTETAQIWVEKYKEFGFKKKNIGFLFEEGVFE |
| | | LMKLKYGNDEASQIEKNGEVLSIFDGWKGFLGYFKKFFETRNNFYKD |
| | | DGTSTAVSTRIINENLKIYLDNLIKYNKIKDKVDFKEADILQENKLNLS |
| | | DFFNVESYAKYSLQKGIDYYNEILGGKTLKNGTKLKGLNEVINEYKQK |
| | | NKSGELSKFKMLKKQILGEGEDRTLFEEIENEDELKDVLKDFFYNADP |
| | | KITLFKTLLEDFFSNTEKYKDELDKIYFNTVAINGILHRWVDDSGVFQ |
| | | KYLFEVLKSNKLVKSNHYDKKEDSYKFPDFISFEHIKVALENCERDGLK |
| | | DKFWKEKYYTKECLTENGLANLWQEFLEIYKCEFKKLYDYKTDDNDC |
| | | YLQYRDNYKKYILDANFNPKEKSAKDIIKDYLDSVLSIYQLAKYFALEK |
| | | KKVWTTDYETGDFYYEYIKFYEDTYEQJIKPYNLVRNYLTRKPINTAKK |
| | | WKLNFDNAYLASGWDKDKEVSNLTVILRRDEQYYLAIMKKGKNKIF |
| | | EKKFSCGEFEKMEYKQIAEASSDIHNLVLMNDGSCRRCIKMHDKRK |
| | | YWPLDISIIKEKKSYAKENFVRRDFERFVNYMKKCSLLYWKEYDLKFS |
| | | DTSTYKNINDFTNEIASQGYKLSFSAIPESYINEKNNNGELYLFQIYNK |
| | | DFGIKTEGNKNLHTMYWESIFSEENRFRNFIVKLNGKAEIFYRPKSEQ |
| | | VEKEQRNFTREIIKNRRYTENKIYFHCPITLNRISRENVKKFNNGINNY |
| | | lATNPNINILGVDRGEKHLVYYAlVDQDGKLIDAEDATGSFNTIGSTD |
| | | YHRLLEEKAKDREKERKDWDLIRGIKDLKKGYISLVVRKIADLAIKYNA |
| | | IIIFEDLNTRFKQIRGGMEKSVYQQLEKALINKLSFLVNKGEKDPEQA |
| | | GHLLKAYQLAAPFQTFDKMGRQTGIIFYTQASYTSKIDPITGWRPNL |
| | | YLKYRNIDDSKESIKKFKSILFNKEKNRFEFTYDLKDFVDFEEDKIPEKT |
| | | EWTLCSSVERHKWNRHMNNNKGGYEVYKDLTENFYKLFDENNIS |
| | | MNKDIVDQVESISNGNFFRQFIYLFNLVCQIRNTDEKAEDVDKRDFI |
| | | LSPVEPFFDSRRAKDFKAYGDNLPKNGDENGAYNIARKGVLIIKKIKE |
| | | YYNQNGSCDKLGWGDLSISHKEWDDFATNN |
| 276 | Cas12a | MDSYEQFTKLYPIQKTIRFELKPQGRTKEHFDNSNFLEKDRERDDNY |
| | | KILKEVIDDYHREFIDECLSNIQLNWDDLKKFSEEYRRSKEKKNNRDS |
| | | ESEQKRMSTTSETRAINKKNLEAEQKRMRGEIVSAFKKDDRFKHLFS |
| | | EKLFSILLKNQIYEKGTLEEIEAFDCFNKFSGYFKSFHENRKNMYSDED |
| | | KETAISYRIINENFPKLLDNFEKYQYVCREYPEQIREAESTLAEAGCYIK |
| | | MDEIFSIDNFNNVMMQGGKESGISRYNLAIGGIVQGTGEKPKGLNE |
| | | FLNLAYQNEPNGRKKIRMEPLYKQILSKEESFSYRLEAFTDDSQLLSAI |
| | | RSFFDIVEKDKNGNIFDRAVNLMSSFSNYDTSKIYIRKAYLNQVSKEIF |
| | | GYRGKSDSKPAKTADESLNKSGGWEKLGQMLRDYKADSIGDRNLE |
| | | KTCKKVDKWLDSDEFTLSDILGAISLAGSNETFEAYVSEICVARRNIDK |
| | | EKEKEKNINVEKISGDTESIQIIKALLDSVQEFFHLLSPFQLHPNTPHD |
| | | WTFYAEFNDIYDKLSAITPLYNQARNHLTKKNLDTSKIKLNFNNPTLA |
| | | NGWDVNKEYENTAVILIRDGKYYLGIMNPKNKRKIKFDEGSGAGPF |
| | | YQKMVYKLLPGPYRMLPKVFFAKKNIDYYNPSQEIREGYKAGKHKK |
| | | GKEFDKGFCHKLIDFFKESIQKNENWKVFDFKFSPTESYDDISEFYQE |
| | | VEKQGYRMYFVNIPSDTIDRYVEGGDMFLFQIYNKDFAKGAKGNK |
| | | DMHTLYWNAVFSEENLQKGVMKLSGEAELFYRKKSDIKDPPHREG |
| | | EILVNRTYIDRTHVSGVMGEQNTVKESRIPVPDEIHKNLFDYYNHGR |
| | | ELTKEEKEYCDKVGSFKAYYGIVKDRRYLENKMYFHVPLTLNFKAIGE |
| | | KRINKMAIEKFLTDENACIIGIDRGERNLLYYSIIDRNGKIIDQKSLNVI |
| | | DGFDYHEKLSQRQTEREVARQSWNSIGKIKDLKEGYLAKAVHEISK |
| | | MAIKYNAIVVLEDLHFGFKKGRLKVEKQIYQKFEEMLINKLNYLVFKD |
| | | VSDSSDAGGVLNAYQLTAPLESFSKLGKQSGILFYVPAAFTSVIDPTT |
| | | GFVDLFNSSSITSTQKKKEFLQRFESIVYSARDGGIFAFTFDYRNFSKIA |
| | | TDHRNMWTVYTHGERIRYVRDEKCYKTTDPTKRIKEALSGIEYDDGS |
| | | DIRDKITQSGDNNLINTVYHSFMDTIKMRNKDGRIDYIISPVKNRNG |
| | | EFFRSDYKHRDFPVDADANGAYHIALKGELLMRMIGKTYDSNSDK |
| | | MPKLEHKDWFEFMQTRGDQ |
| 277 | Cas12b | MCVSRLPWFNITLTGKLNRQRLNQMCVSRLPWFCTPKGQLAATPK |
| | | TVVAQQENAMLAIIRDVHEAAPADLKTVAQRLEPGYFVTQFPKQQ |
| | | MTGDEARAEAERLFAACQKKFKELAEYEDGYRQCLDALGPNLSLPR |
| | | LGRKPKGAYPYAVVFKLMPTNATWECFKRVTASLYKRAQKGVVSP |
| | | VSADSIADVRTNDEPLFEYFTNLALVRPPGNKDRAVWFEFDLAAFIE |
| | | AIKSPHQFFQDTIKREQAVAQIKAKLDAMDGQGRAASGEEDALPGF |
| | | EGDDRITLLRELVTDTLGYLAEADASTSPGGKIEYSIQERTVRGFAEVK |
| | | RRWRDLVEKGKATEDALLKVLAEEQTEHRDDFGSATLYRELAKPKF |
| | | QPIWRDPGTQPWHADDPLRAWLEYRELGRELEDKQRPIRFTPVHP |
| | | VHSPRFFIFPKKKGGGRFGTVHEPGQLRVMAGIVAQTQHGWEPVP |
| | | VRITYAAPRLRRDQLRDDVETDLESRPWLQPMMQALGLPEPDTAD |
| | | FSNCRVTLQPSAPDDIQLTFPVDVSADKLTTAIGKAARWAKQFNLFP |
| | | DGDNFYNASLRWPHEKKPSKPPVPWHEALDNFSVLAADLGQRCA |
| | | GAFARLEVRANDDFAGKPSRFIGETPGKKWRAALVAAGMLRLPGE |
| | | EQTVWRPGATGPNFHTELSGSRGRMARPHEADDTADLLRAFDCPE |
| | | ESLMPADWRTSLSFPEQNDKLLVAARRYQSRLARLHRWCWFLTDE |
| | | KKRQTALDEIREAEDMPAADDPQLTDKLRALLLQKQAALPGLLVRL |
| | | ANRILPLRGRSWQWETHPDKADCHLLTQTGPALPDVWIRGQRGLS |
| | | MQRIEQIEELRRRFQSLNQMQRREIGGKPPIRRDDSIPDCCPDLLDK |
| | | LDQIKEQRANQAAHMILAEALGLRLAPPPADKRQLRASRDVHGQY |
| | | VKSREPVDFIVIEDLSRYRSSQGRAPRENSRLMKWCHRAVRDKLREL |
| | | CEPFGIPVVETPAAYSSRFCSRSGVAGFRAVEVGPGFDREFPWMML |
| | | KDREDEGEPVRQLILQVATLNQGRDGKPPRTLLAPLAGGPIFVPIVD |
| | | KLNGADIQPALAQADINAAINLGLRAIADPRLWSIHPRCRTQRQGD |
| | | QMLTREKRKFGETGQPLAVHRADGVKPDDTRNPNFFADISGSLPA |
| | | WESATLDGQHLLSGRCLRSEIKKRQWQRCAEINDRRMNRWMKGE |
| 278 | Cas12b | MTELQTQRAYTLRLKGIDEKDQSWRDALWKTHEAVNKGAKVFGD |
| | | WLLTLRGGLDHTLADAEIPGEKGKPDRAPTQEERKHRRILLALSWLS |
| | | VESERGAPEEFIVATGKEPAATRNDKVIAALKDILRGRNLTEEKISEW |
| | | TEVCTPSLSAAIREDAVWVNRSRAFDEAVKRIGSSLTREEVWDMLE |
| | | CFFGSRNAYLAPVKISEDESSDGEQEEKAKDLVQKAGQWLSSRFGT |
| | | GEGADFAKMAAVYAKIAAWAGNAQAGTTGNEVINNLATALREFT |
| | | PKSNDLKGVLDLISGPGYKSATRNLLKQIANTKTVTREDISKLQETAG |
| | | EDSEECATKTGSKGKRAYADAILKDVESVCGFTYRIDKDGQPVSVAD |
| | | YSKYDEDYKWGSSRHKEFAVMLDHAARRVSLAHTWIKRAEAERRK |
| | | FEEDSKKIMQVPQAAKDWLDAYCAQRSEASGALEPYRIRKRAIQG |
| | | WKEIIASWNKPDCKTAEDRIAAARQLQDDPEIEKFGDIQLFEALAED |
| | | DAQCVWKKEDGTLDPEILINYTLASEAMFKKQHFKVPSYRHPDAFL |
| | | YPVFCDFGNSRWELDFSIREAATKLKEIEAKIEKQRQEVHKVQQALE |
| | | KCENDEKRPKMEERLKEAQKKLQESQNYGEYLHSNNKITMVLFDGT |
| | | FVKKHIFAWQSKRLTKDLALYQEPSADPKNVVSRADRLGRAVASVG |
| | | INDAVKVAGLFEQENWNGRLQAPRQQLEAIAQYVEKHGWDNKAE |
| | | KMRASIKWFITFSAKLQSKGPWNEFARKHGLKEDPHYWPHAEKNE |
| | | NRTAHSRLILSRLPGLRVLSVDLGHRYAAACAVWEALGSEAFKKDIE |
| | | GKRIIRGDTDENALYCHTEHEANGKKHITIYRRIGADTLPDGAHHPA |
| | | PWARLDRQFLIKLQGEDEQAREASNEEIWKVHQLENTLGRRTPLID |
| | | RLIAGGWGYTEKQKARLEVLTNLGWCPTNKTDNQEEGDEEETAILS |
| | | KPSLLVDDLMFSAVRTLRLALKRHGDRARIAHYLITDEKTKPGGVKEK |
| | | LDKNGRVELLLDALGLWHDLFSSPGWHDEKAKQLWNAYIAGLLPE |
| | | GELQQAKSVTTSAALGGQQKKEKKEKLRAVAEALYLNSDLCHSLNE |
| | | VWRKRWEEDDKQWRIYIRWFKDWIMPRGANAKSPAIRHVGGLSL |
| | | TRLATLTEFRRKVQVGFFTRLHPDGTKTETREDFGQKTLDTLEHLRE |
| | | QRVKQLASRIVEAALGIGSEDKRHWDGKKRPRQRIADPRFVPCHAV |
| | | VIENLTHYRPEETRTRRENRQIMEWASSKVKKYLSEICQLHGLHLRE |
| | | VSAAYTSHQDSRTGAPGIRCQDVSLIEFMKSPFWRKQVAQAEKKQ |
| | | KEGKGDAVERYLCELNQKWKGASEEEWRKAGFVRIPLRGGEIFVSA |
| | | AGHDSPAAKGIHADLNAAANIGLRALLDPDWSGKWWYVPCNSST |
| | | MCPARDKVTGSAAVNPGQPLQVSAQLESDDAAKDTKKRKKKGDG |
| | | KSKEIINLWRDISSYPLEDTRGGTWSNKTVYWNRVQSNVVHILQNQ |
| | | MKG |
| 279 | Cas12b | MPETTQRAYTLRLQGHDPKDASWREALWKTHEAVNRGAKAFGD |
| | | WLLTLRGGLDHSLADEGAPGQTPTEEQRKQRRILLALSWLSVESEN |
| | | GAPQEYIVPHDRDNESGARQNWKTREALREILKNRGCRDDEIESW |
| | | CHDCEPSLTSAIRKDAVWVNRSKAFDNAVQSIPNFSREEIWDLLGCF |
| | | FVSSQAYLAPLESPKDDKPDASKKDSSKDLIQSAGQWLSRRFGRGK |
| | | GLNFARLAETYEAIARWASVANPGDTNDLIADLAKTLNAETPELDGI |
| | | LKVVSGPGHKSKTRNLLRSLSAVNHITKDTLQRLKDTANEDAKKAKL |
| | | KKGEKGHRAYAYKVLEAVEDACGFTYLQEGDRAKHCEFAVMLDHA |
| | | ARRVSSLHTWIKRAEAERRRFEIDTKKKDQLPPSVKEWLDTYCQKRS |
| | | KETGAVEPYRIRRGAIEGWKEIVEAWSKAGTTTAEDRKHEARRLPD |
| | | NPHIDKSGDIKLFEDLALEDALPVWHANGDPNNPPDPQLLIDYVEG |
| | | SEAEFKKRAFKVPTYCHPDPLVHPVFCDYGCSRWNVSFAIQPVKKQ |
| | | KLSSEEKLPAKGLLLDLLHGTAIRPVALRWQSKRFARDLALNTTDSSD |
| | | KPNEVTRADRFGCALAKCPSSQKIRIRGLFEEKYWNGRLQAPRPELT |
| | | ALAKRVAKYGWDKKARKLRNSLNWFITFSANLRPSGPWEEYTKYAE |
| | | KAFSSNASAKPSVSRGGFWVVHASPNKRGKMAQLRLCRLPELRVLS |
| | | VDLGHRYAAACAVWETLSKSAFEQEIHERKILRGGTGPNDLFCHTQ. |
| | | HDTNGQSKVTIYRRIGADTLPNGTPHPAPWARLDRQFLIKLPGEERE |
| | | ARKASPTELANVEKLEKELGLKTSENRVKRIDDLMSDTLRTVRQALR |
| | | RHSLRARIAFNLATLRDQSDGDEESQSKQKRDTRWNNTVKIWHSLL |
| | | ESNEWTDDWAKALWDELGPLSDPQKADDAEWLKLAAEKFYTRW |
| | | QEDEQTWRERLRWLRRWILPRGSQAASQKGSIRHVGGLSLTRLATI |
| | | KTLYQVLKAYHMRLKPDNSRKNIPAEGDEALQNFGQKILDDLEHMR |
| | | EQRVKQLASRIVEAALGLGRMKQVTIGKDPKRPREPVDQSCHAVVI |
| | | ENLTHYRPEKRQTRRENRQLMDWSAAKVKKYLKECCQLHGLHLVE |
| | | VSASYTSRQDSRTGAPGIRCQEVPLTDFLKKNFWREQVKQAKQRLS |
| | | EGKANARDRYLCQLNERWGNAPAPVTQTAIRLRIPLNGGELFVSAD |
| | | QNSPASKGIQADLNAAANIGLRAITDPDWPGAWWYVPCEANTFK |
| | | PVKDKVAGSAAIDSNVSLKKDSPNSEKPASDRKSRTSKSMINLWCD |
| | | TSSKSLSEKDQWQESAPYWEDVAARTINILQASLACSTTNSQ |
| 280 | Cas12a | MKSLAQFQNLYALQKTLRFELKPEGHTRETFNRWLEEIEKEQASENE |
| | | NIVYQDLLRAKKYEKIKIILDEYHKDFIEQALAYANLTELEKYEELYRKS |
| | | NRTSEEEEEFENTKESLRKQIANIFIKNPNKTVQERWKFLFSKKLIQNE |
| | | LIVWVKGNYELLSEKLKNEFPDESSIISTIEDFKYFTTYFRNYHENRKNL |
| | | YSNEDKFSTIAHRLIHENLPKFIDNIAIYQKAKAVLNINEVEKELGLPED |
| | | TLDKIFSLDFFSKALTQKGIDQYNYFLGGKTENEVKKIKGLNEFINLYN |
| | | QQQQDKNQRLPFLKVLYKLPLFERTSTSFRFEPIENDRDLIERIGKFYY |
| | | NDLKQYRDDSQGDTTDILSGINTLLRHVHDYRDGLYVNGGITLTQIS |
| | | QKIFGSWSYINNALAYFYDTYIDASGVDHQGERKPKKQKQIQEKTK |
| | | WLKQKQFPVILVEKALSEYKSIETNEDLKTRISDTTLCDFFKRCGNDD |
| | | NGQDLFDRIEARLREKNEEGYSLEDLLKKEFTTERKLMQDKTKTLLIK |
| | | NFLDVIQGDKDDITAGLLHFVKCLIPRTEISEKNELFYSGMEKYLNILS |
| | | EVTPLYNKARNYLTQKPYSIEKVKLNFENSTLLDGWDENEESDNSCV |
| | | LLRKRGYYYLGIMNKKHNMIFDRKIYPKATEGEAYYEKMIYKLLPGAY |
| | | KMLPKVFFSEKNIDYFKPSEEILRIRNTASYSKNGQPQEGYQKASFSIE |
| | | DCRKYIDFFKKCIANHWDWQKFNFNFSPTEYYQSIDEFYREIERQGY |
| | | KIDFVKIPESYINQLIKENKLYLFKIYNKDFSEKKKSKGKDNLHTLYWK |
| | | MLFDEKNLKDVVLKLNGEAEVFFRQKSILYNEEIWNKGHHYSELKDR |
| | | FSYPIISNKRYAEDKFFLHVPITLNFKADGINNVNNMVNEFIKDNRDI |
| | | HIIGIDRGERHLLYVSVINQKGDIVEQCSLNEIVTEYNGKIFKKNYHEE |
| | | LDNLEKERDRARKDWQTIANIKELKEGYLSHVIHKISKLILKYNAIVV |
| | | MEDLNSGFKRGRQKVEKQVYQNFEKQLIEKLNYLVLKESNVDEPGG |
| | | VLRAYQLANKFETFKKLGKQSGIIFYVPAAYTSAIDPVTGYIQYLYPLK |
| | | QADSVEKARKFYSQFKRISYNPHKQWFEFSFDYNDFNIIYHGKSSWT |
| | | ICTTNTERYMWNRLLNNGHGGEELVYVTNELELLFGEYNIIYGDGKD |
| | | IKQQITDVQDIDVDRTAKQFYKRINELLNLTLKLRHNNGKKGADEED |
| | | YILSPVEPYFDSRFESRKPSMQQTLPINADANGAFNIARKGLLLLERL |
| | | NQLGVEEFEKTKKSNNKKTQWLPHELWVEYAQNHTRK |
| 281 | Cas12b | MAYQNGKEQPTVTNQRAYTLRLSGTNDQDSIWRNRLWHTHEAV |
| | | NKGAKTFGDWLLTMRGGLCHTLAEADVPGKGNKPARHPTPQEIRS |
| | | RRVVLALSWLSVESQHGAPERHLVSHDLDIATGERKNWKTVEALRE |
| | | ILHGRCLCKELIDEWANDCRDSLSATIREDAVWVNRSKAFDLAAKKI |
| | | GASLTREELWDFLQPFFANKHGYLQMDTVAGVTNGDSETDAEEAK |
| | | EDSSEEKAKDLSQKAGQWLSSRFGTGTGADFSRFSKVYEVLAARCG |
| | | SVAVGVSGVEAIRILAGTLADFSPGSNDIEGMLGLMSGPGYKSATR |
| | | NILQKINTLQTVSQQDLDRLREASEKDALQSKQKVGGKGSRPYANAI |
| | | LQDVEAACGICYAGTGESPARHWQYAVILDHAARRVSMAHSWIKR |
| | | AEEQRSKFEIEKDKLDHVPKDALAWLDAFCARRSSESGASDAYRIRR |
| | | SAVDGWKQVVAAWAALPPKPENQGSELLSDAESARIQAARELQDT |
| | | VEKFGDIQLFEALSLTGAKCVWQPDGRPDAQPLLDYVAGTDAISKK |
| | | QRFKVPAYRHPDALLHPVFCDFGNSRWNINYAIHRAPEKLTPAQQL |
| | | LEKKKAEIDKAELTLAKAGDAAKQANISEKINGLRAAFIQQQEKVAW |
| | | LNSRHAMTMSLWDGTHIEDTPLIWQSKRFGSDIGQPVEAQPLPVS |
| | | RADRFGRAVALAQDNVPVIPSGLFDLSDWNGRLQAPRRQLEAIAAl |
| | | RDSAKLSVNEKQQLVAKRIQSIRWLLTFSAKLQSHGPFIAYAAQHGF |
| | | DWRYGAHGPENKSRQGLAKLILCRLPGLRILSVDLGHRYAAACAVW |
| | | ETLNAGQIQKACLDAGKEAPGPCTLYLHLKQIANGKEKKTIFRRIAAD |
| | | TLPDGSPHPAPWARLDRQFLIKLQGEDRDARLATSEEIAAVEQMEN |
| | | ELGVVRQLKRKGRELLVDELMSDALRTLRLGLRRHGVRARIAFNLTA |
| | | NKRIRPGGKEEVLDQEGRVLLLTETLLAWYELYTAERWTDEPAREL |
| | | WNRHIQPLLGATILQNTVNQEDTPSAAKRRKLREETSGKLKHVAEEl |
| | | AKNDSLCRQLHVLWSAQWQTEDVIWRTRLRMMRRWLLPRGVKR |
| | | NAQLRISIRDVGGLSLTRIASFKSLYQVQKAYQMRPHPEDPRLNIPER |
| | | GDSRLENFGQRVLDAMERMRENRVKQLASRIAEAALGIGGETGISS |
| | | KDGSQKKRPTERSSDPRFAPCHAVVIEDLTHYRPDETQTRRENRQL |
| | | MSWSSSKVKKYLGEACELNGLYLREVSPAYTSRQDSRTGAPGLRCN |
| | | DVTVVEFNNSPFWRKQVGAAEKNQKEGNKGDARERYLLSIEEGIRG |
| | | AANDRDIFRIPVKGGEIFVSACITDGGNNAKKNAPPGLQADLNAAA |
| | | NIGLRAIFDPDWEGRWWYIPCDAATLCPDAKKFIGCKAVDPTKPLR |
| | | VVAEEGAISASGIGSKKSGRKKNAATDGTRIVNLWRDPSGAPIHRD |
| | | VLRSPEWQDYAGYWNEVQHRVIRNLKTCYEQTSQQEDPFVSQDA |
| | | DKPF |
| 282 | Cas12b | MKRLAETALADKVKCETNSRPKGERAYANSILHDVESACGFTYRVD |
| | | KGEQPVPVSDYSHYANDYRWGPANHSEFAVMLDHAARRVSLAHT |
| | | WIKRAEAERRQFEENAKKIDKVPKVAREWLDSLCAERSIVLGALEPY |
| | | RIRRRAVDGWKHVVAAWSKSDCKTAQDRITAARLLQEDPEIDKFG |
| | | DIQLFEALAEDHAVCVWQRDGEAGKTSDPQLLIDYALAAEAEFKKR |
| | | HFKVPAYRHPEAFWHPVFCDFGQSRWKICFDVHKNRQSRRQRAC |
| | | ANRISRKICFDVHKKRQTLRLSLEVWTGSKMLDMPLCWQCKRLAR |
| | | DLALGQDHKKDRSCQVTRADRLGRAVSNVARNQEVQILGLFEQEY |
| | | WNGRLQAPRPQLEALGRYIEKHGWDAKAQKSCRAIRWMISFSPRL |
| | | QPAGPWGKFAEKLQLNPNPKYWPHAEDNKDRGSRSKLILCRLPGL |
| | | RVLSVDLGHRYAAACAVWEAVDAEQVKEACQAAGHREPNENDLY |
| | | LHLKKRTTKQKKGSQGVVEETTIYRRIGADTLPDCTPHPAPWARLDR |
| | | QFLIRLQGEEDEARAASNEEVWAVHKLEAELGRTIPLIDRLLGAGW |
| | | GQTEKQKARLKALRELGWTPANKCQAFNSTDETELRRPSLAVDEL |
| | | MLDAVGTLRLALKRHGDRARIARYLITDERTKPGGVKEKLDENGRIE |
| | | LLQDALIIWHGLFSSPRWRDDAAKQLWNEHIAKLVGEQNLVEVSED |
| | | ASGSERRTKQKQNREKLREAAKALVDDVALRQALHDMWKRRWEE |
| | | EDREWRRRLRWFKDWVLPRREQARKAYSRPAETGSSSHPKRRARY |
| | | AAIRRVGGLSLTRLATLTEFRRKVQVGFFTRLKPDGTKAEAKEGFGQ |
| | | STLDALEHLRAQRVKQLASRIVEAALGVGRIRRFPGVKNPKRPDTPV |
| | | DKPCHAIVIENLTHYRPEETRTRRENRQLMTWSSSKIKKYLAEACQLY |
| | | GLHLREVTAAYTSRQDSRTGAPGLRCQDVPVKEFMRSLFWRKEVA |
| | | QAEKKLTAGKGSSYERLLCELNQRWKDNSPGDGKRAELLRLPHKGG |
| | | EIFVSAAPDSPAARGLQADLNAAANIGLRALTDPDWPGKWWHVP |
| | | CNAVTFRPVEDKVKGSAAVKLDQSLRQVAHPQSKDPGAKKSKEIVN |
| | | LWCDISSLPLEHREWKLDWEPYPAYWNNVQCRVIRVLQGKV |
| 283 | Cas12b | MANAKVKTTTRSYTLSLNAPSDTTDRSPLWHRIFRTHYAICCGAREF |
| | | GKLLLDLRGGLPTSLAQLGEGIAENDRRQTQRGTRRILALGWLSVED |
| | | LDHARNDPHRVQDTAPGSPLDQDLAEKILRKILITKGIKSEEEQSNWI |
| | | SDCLPALTANIRPDAVWVNRAESFAQWQRGTQPGAQPPTPEEAQ |
| | | QILFSLCGESLVTLTLPEQPAAAGQKQPDQETSPDPEEQTDRPPAAP |
| | | SADDEMDPSNASRGIFGDLFGENAEGKRSRSQGKDNFACAVRDFL |
| | | CANPTPSADAITEFREKQKPREPNPPGPEKYPPEVSTSGAPTAVAKR |
| | | YRKLLVCAGLWPKSADEDGSSRNSAKTKFADPKEPQKTEIQINALDLI |
| | | DACNQAAPADDSGTSPKAGRVFAPAWASNIAEKVASATQMPANA |
| | | KSLNEFKRLMFALAARRFSQTQSWTRRNEAERHMAAARQDAAVA |
| | | RLREIDPDHKAQDWLRGYEQRRADQSGSNGEFRITRRMIGEAEAV |
| | | FKAWAGTNSAAERELKTVAVQTTAEKFGDAALYSEIARNTAAEAV |
| | | WRSGSAPEILDQWVKLRKAQSDQQRTRVPRFCHPNAFRHPTWCE |
| | | FGESSKPGVWYAWNPKSKPRKPEVGGEGDGTRRLWVLLPDFNSGI |
| | | GQAVPLRWRSKRLSKDLGEALQPSDAPIPRADRVSIAAAGLNLEGA |
| | | NGVPARYRPSLPFSENTKGWNARLQANRTALLHLESKWDAEAAT |
| | | WRDGGRSLLALKWFTTFSPELAMSEGPGRAIHPKLGWNSEPHSDL |
| | | NRAQKRGGNAKLILSRLPGLRVLSVDLGHRYAAACAVWETLTTEQ |
| | | MNAACQAKNHTQPAESDMYVHLAHPTERVVKSGRKKGQNLIQTT |
| | | VYRRIAADTLPDGTPHPAPWGRLDRQFLIKLQGEQRPTRAASKNEA |
| | | DLANALFHRLGLRSDADSENKSRAVDKLMARTVRVATLGLKRHARR |
| | | AKIAYALDPNTKAIPGMGGSSAAFTPGDEPHIHLLTDALFDWQSLAT |
| | | DAKWDDAHARSLWNHHIATLPGGFHLENPTPRDESAHEPSRQRQ |
| | | RSGDDALRATLKPIAEKLSKADRQEVHAAWKKYWGDSDGQSAIVP |
| | | KVLQGQRGPEKTTPSASASGWHGKIRWITDWIMGKYLEGCTGHA |
| | | WKHDVGGLSVSRITTMKSLYQLHKAFAMRATPEKPRGAPEKGESN |
| | | LGAAQGILTAMESMRQQRVKQLASRIAEAALGAGIERRSDNGREL |
| | | QRPRERVDDPRFAACHAVVVEDLTNYRPDEMQTRRENRQLMQW |
| | | ASSKVKKYLSEACQLHGLYLRGVPAGYTSRQDSRTGAPGVRCGDIPV |
| | | EELMAAPRWRRQILTAEKTRRENNTGTARDRYILTLDEKYRLLTAEQ |
| | | RKKTPPARIPVKGGDLFVSADPDSPAASGIQADLNAAANIGLKALID |
| | | PDWPGRWWYIPCDATTHKPSPERTRGSAAVDCDVPLGPDSTGTPE |
| | | DRDAKPKKNQRNSKIAGRGQSAIINLWRDPTHLPIKENPSAWCESK |
| | | KYWNQVEHNVVKVIESKGQKLTQTAEAATGESASSPPIAPTDVPW |

Those skilled in the art will appreciate that, given that their amino acid sequences are known, these enzymes can readily be produced (e.g., through culturing of source organisms and/or by recombinant expression/purification, as, for example, may be contracted from any of a variety of commercial sources). Produced enzymes can then be assessed for direct and/or collateral cleavage at varying temperature(s) and/or for other evidence of stability and/or functionality at relevant temperature(s).

### Example 4: Thermostability of Cas13

This example confirms and further demonstrates the thermostability of Cas13 enzymes. This example provides certain thermostable Cas13 candidates for use in improved collateral activity assays as described herein. The thermostability of TccCas13a and ThpCas13a was tested. Briefly, varying ranges of labeled RNA target was incubated with TccCas13a or ThpCas13a; Rnase Inhibitor; T7 RNA Polymerase, MgCl2 and a cRNA (either in forward or reverse complement orientation). FIG. 3A demonstrates the significant activity of TccCas13a at 65° C. The data of FIG. 3A also suggests the structure of the cRNA can influence the activity of thermostable enzymes. Further, FIG. 3B demonstrates that TccCas13a is active across a wide range of temperatures including the range 40° C. to 65° C.

Additional assays were performed with TccCas13a with or without some components to identify parameters contributing to background within the assays. FIG. 3C demonstrates that presence of the complex of the Cas enzyme and a cRNA contributes to background in the assay.

### Example 5: Exemplary Discovery and Screening for Thermostable Cas Enzymes

The present example demonstrates an exemplary method of discovery and screening thermostable Cas enzyme candidates (e.g., Cas12 and Cas13 enzymes) (FIG. 4). Novel Cas12 and Cas13 enzymes were discovered using a custom-built in silico pipeline. In brief, publicly available microbial genomes and metagenome databases were first filtered on the basis of environmental metadata such as sample collection temperature and sequencing read quality. CRISPR repeats were subsequently identified in the filtered genomic datasets using published repeat annotation methods. Next, all coding sequences were annotated in genomes that had CRISPR repeats using published open-reading-frame (ORF) discovery methods, and these ORFs were subsequently classified using a Hidden Markov Model (HMM) that was pre-trained on known Cas12 and Cas13 enzymes. An enzyme was annotated as a putative candidate if and only if its direct-repeats were conserved (>95%) and the predicted enzyme had a domain topology consistent with previously discovered enzymes.

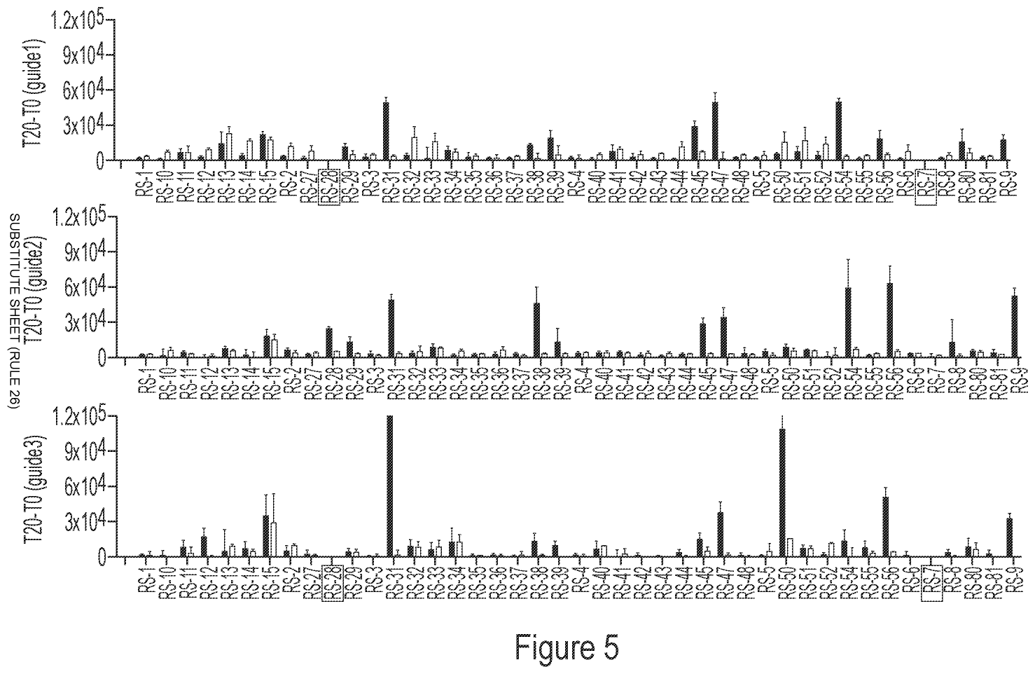

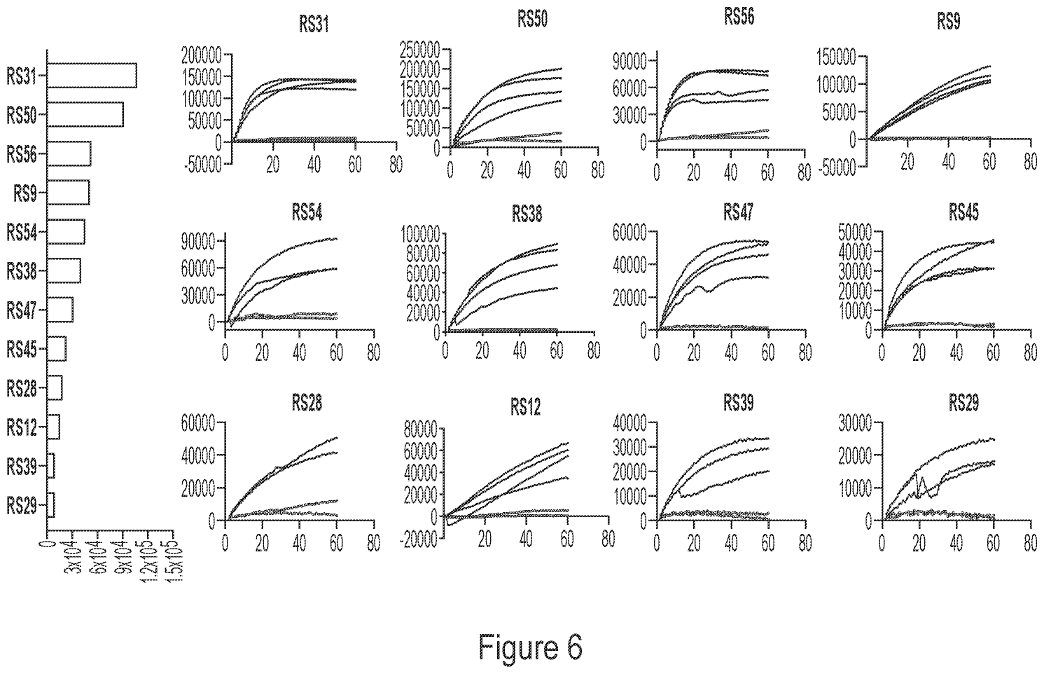

Candidate enzymes were expressed by in vitro protein synthesis (e.g. PURExpress in vitro Protein Synthesis Kit by New England BioLabs) according to manufacturer's instructions. An initial pool of Cas12a candidate enzymes were assessed by endpoint (FIG. 5) and kinetic analyses (FIG. 6) using no template as a negative control for activity at 52° C. Each candidate was tested with three different guide/target pairs at 52° C. (FIG. 5).

A subset of candidates (e.g., 12 candidates) that demonstrated highest activity among the 44 initial candidates at 52° C. were selected in combination with their most efficient guide and target for further assessment at higher temperatures (e.g., 58° C., 60° C., 62° C.).

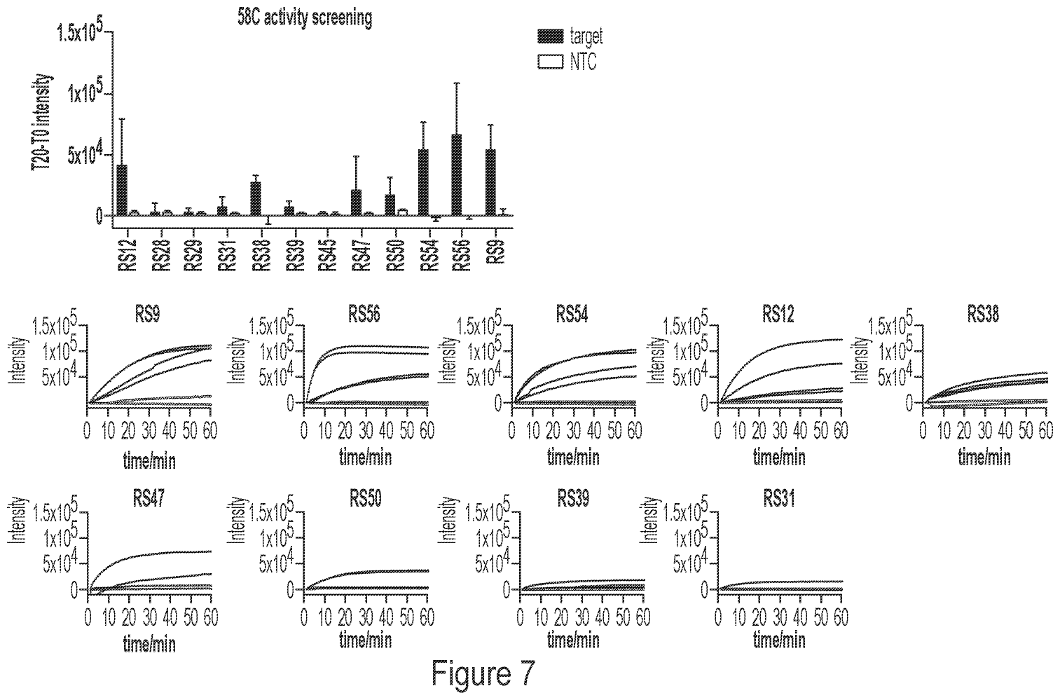

Both endpoint and kinetic analyses indicated a subset of candidate enzymes (e.g., 9 of 12 candidate enzymes) that displayed some activity at 58° C. Of the nine with some activity, five were classified as high activity (RS9, RS12, RS38, RS54, and RS56), and the remaining four were classified as lower activity (RS31, RS39, RS47 and RS50) (FIG. 7).

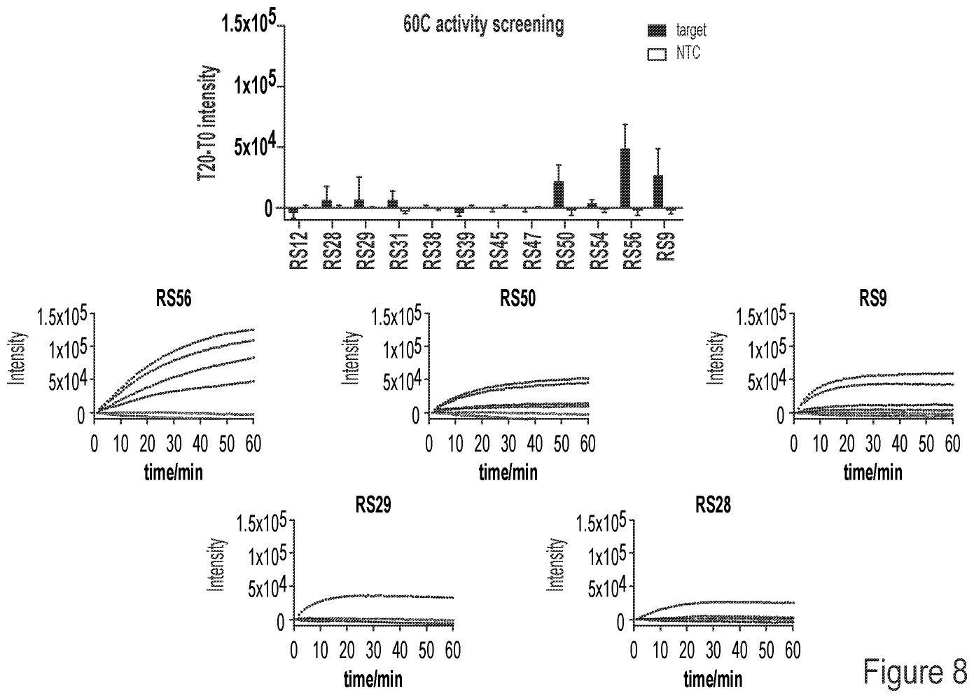

Both end point and kinetic analyses indicated a subset (e.g., 5 of 12 candidate enzymes) that displayed some activity at 60° C. Of the five with some activity, three were classified as high activity (RS50, RS56, and RS9), and the remaining two were classified as lower activity (RS28 and RS29) (FIG. 8).

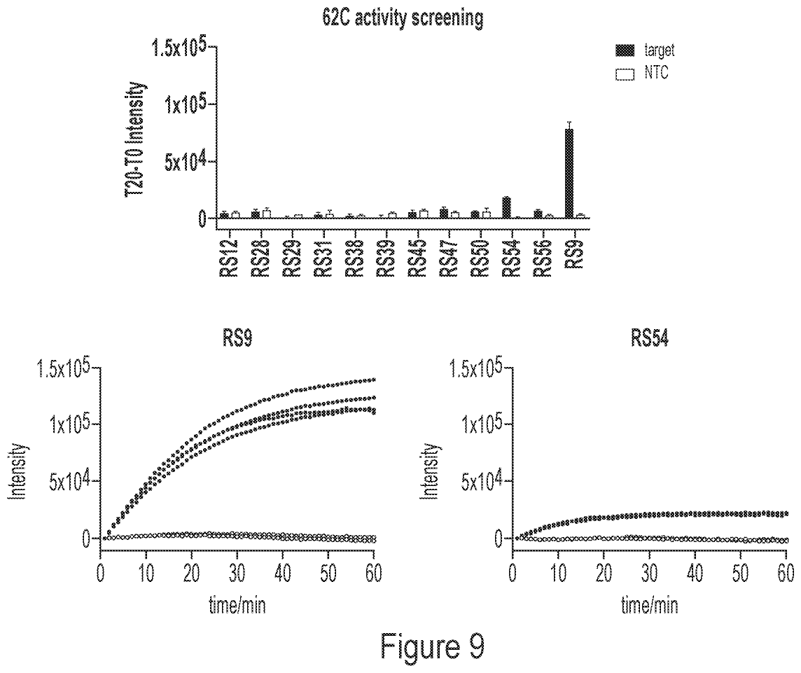

Both end point and kinetic analyses indicated a subset (e.g., 2 of 12 candidate enzymes) that displayed some activity at 62° C. (RS9 and RS54) (FIG. 9).

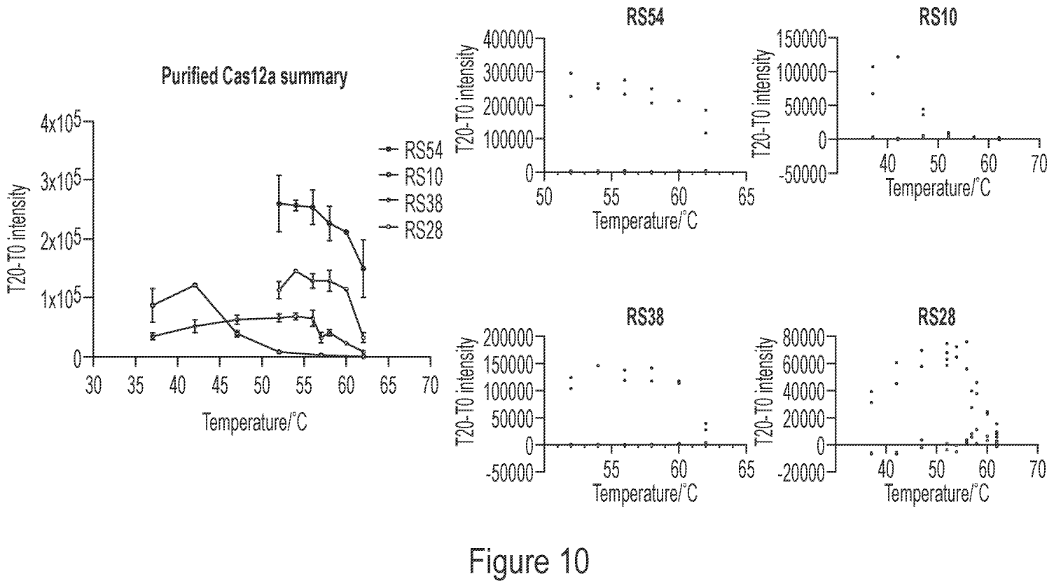

While the refined list of 12 candidate enzymes were assessed at varying temperature, four priority candidate enzymes (RS10, RS28, RS38, and RS54) were purified and assessed for activity at varying temperatures (e.g., approximately 35° C. to approximately 65° C.). RS54 showed activity at LAMP temperatures (e.g., 61° C.) (FIG. 10).

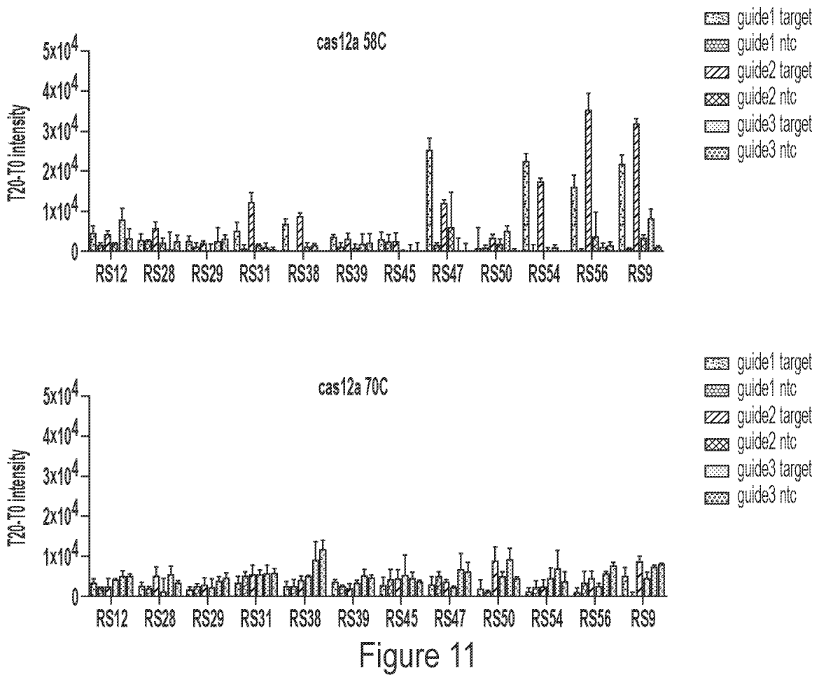

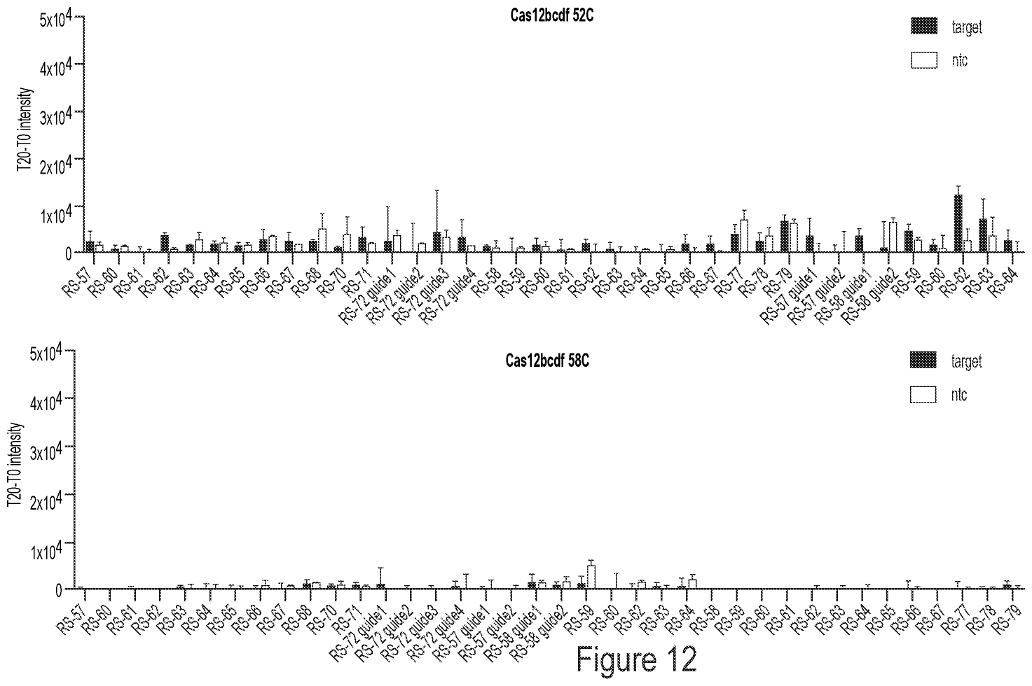

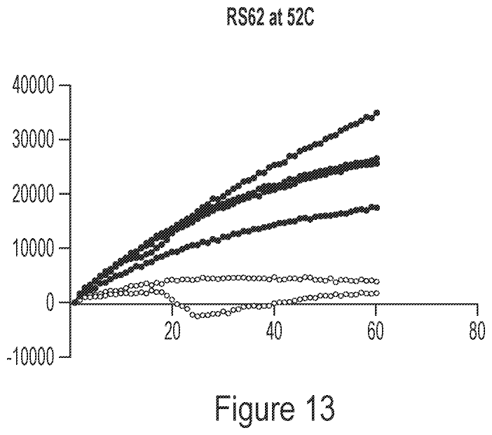

A subset of Cas12a candidates were further examined using three different guide and target sets compared to no template control at both 58° C. and 70° C. (FIG. 11). Cas12bcdf was also assessed with all guide/target pairs at both 52 and 58° C. (FIG. 12). Kinetic analysis were also performed for Cas12a candidate, RS62, which showed activity at 52° C. (FIG. 13).

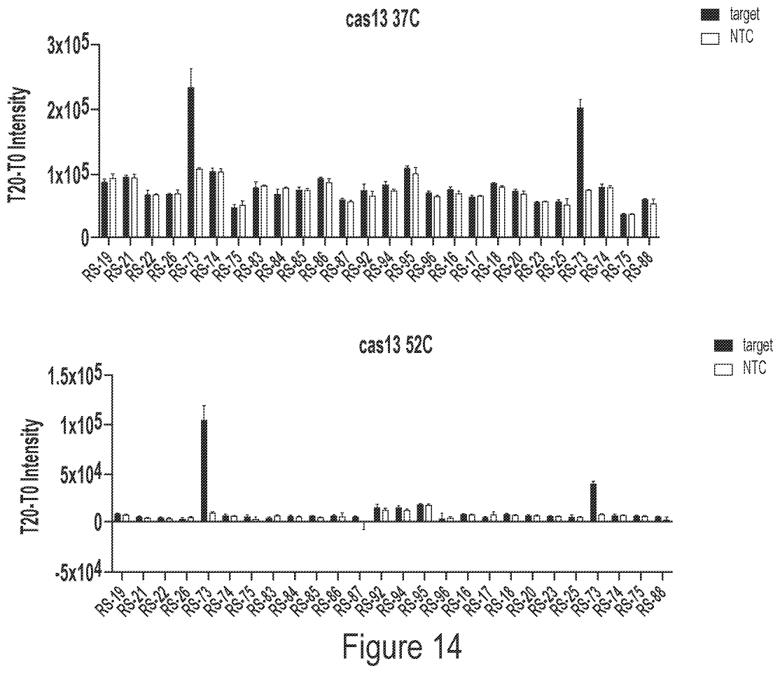

An initial pool of Cas13 candidate enzymes were assessed by endpoint analysis (FIG. 14) using no template as a negative control for activity at both 37° C. and 52° C. A single Cas13 candidate enzyme (RS73) was identified that displayed thermostability.

### Example 6: Exemplary Characterization of a Thermostable Cas12a Enzyme

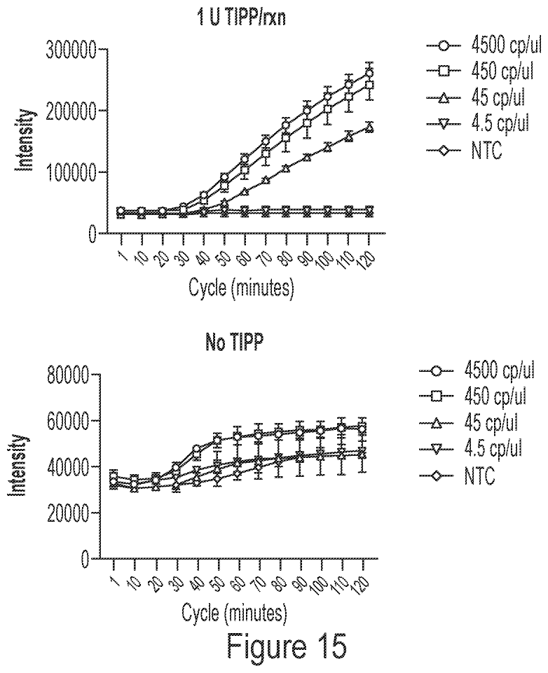

The present example demonstrates characterization of an exemplary thermostable Cas12a enzyme, RS9. To determine whether RS9 required use of Thermostable Inorganic Pyrophosphatase (TIPP) for amplification of a target nucleic acid, real time detection of ORF1ab amplification was completed over a range of starting concentrations of ORF1ab (4.5 copies/μL to 4,500 copies/μL) in the presence or absence of TIPP. An exemplary reaction included 30 ng/μl RS9, 112.5 XL-213 (ORF1ab guide), 1×HKFB (ORF1ab) primer set, 1×wsLAMP mix, 125 nM DNase Alert, with or without 1 U Thermostable Inorganic Pyrophosphatase (TIPP) with indicated viral RNA template concentration present. The exemplary reaction was incubated at 58° C. for 120 minutes on QS5 with detection in VIC channel. Real-time reactions that did not contain TIPP resulted in no statistically significant amplification of ORF1ab compared to the no template control regardless of the starting concentration of template. Real-time reactions that did contain TIPP, with the exception of that with a starting concentration of template at 4.5 copies/μL, demonstrated significant amplification of ORF1ab compared to the no template control, suggesting RS9 requires use of TIPP for amplification (FIG. 15).

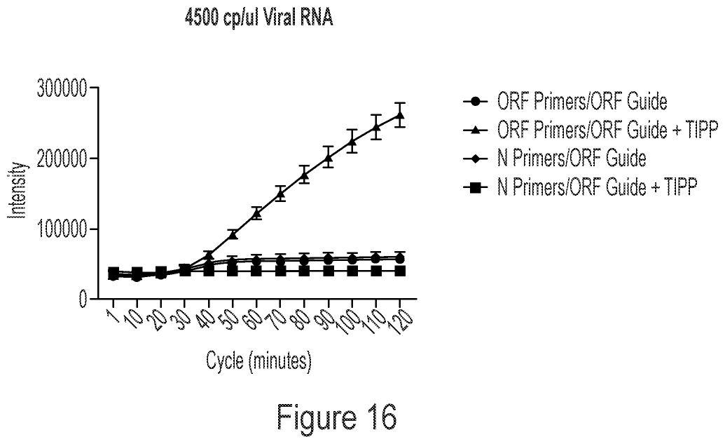

Specificity of RS9 was also assessed using real-time analysis. Amplification was conducted with a starting concentration of 4,500 copies/p L ORF1ab template and either primers and guides specific to ORF1ab or non-targeting primers with an ORF1ab guide. Each reaction condition was also conducted in the presence or absence of TIPP. An exemplary reaction included 30 ng/μl RS9, 112.5 XL-213(ORF1ab guide), 1×HKFB (ORF1ab) or CFB (N) primer set, 1×wsLAMP mix, 125 nM DNase Alert, with or without 1 U Thermostable Inorganic Pyrophosphatase (TIPP), with 4,500 copies/μl viral RNA present. An exemplary reaction was incubated at 58° C. for 120 minutes on QS5 and detected in VIC channel. No amplification was detected for reactions containing non-targeting primers and/or no TIPP. Robust amplification was detected in reactions containing ORF1ab primers, an ORF1ab guide, and TIPP, indicating RS9 is specific for its target and requires TIPP for amplification (FIG. 16).

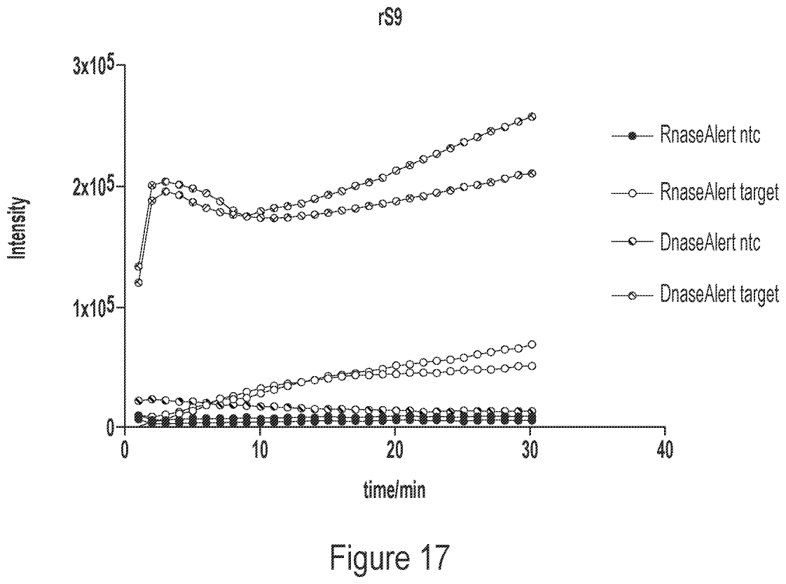

RS9 collateral cleavage activity was assessed using 100 nM of single stranded DNA target and DNaseAlert or RNaseAlert as reporters. A no target condition was utilized as a negative control. RS9 was unable to cleave DNaseAlert, resulting in intensity measurements significantly above no target control conditions. RS9 was able to cleave RNaseAlert, resulting in measured intensity similar to that of the no target control conditions, indicating RS9 has RNA-specific collateral cleavage activity (FIG. 17).

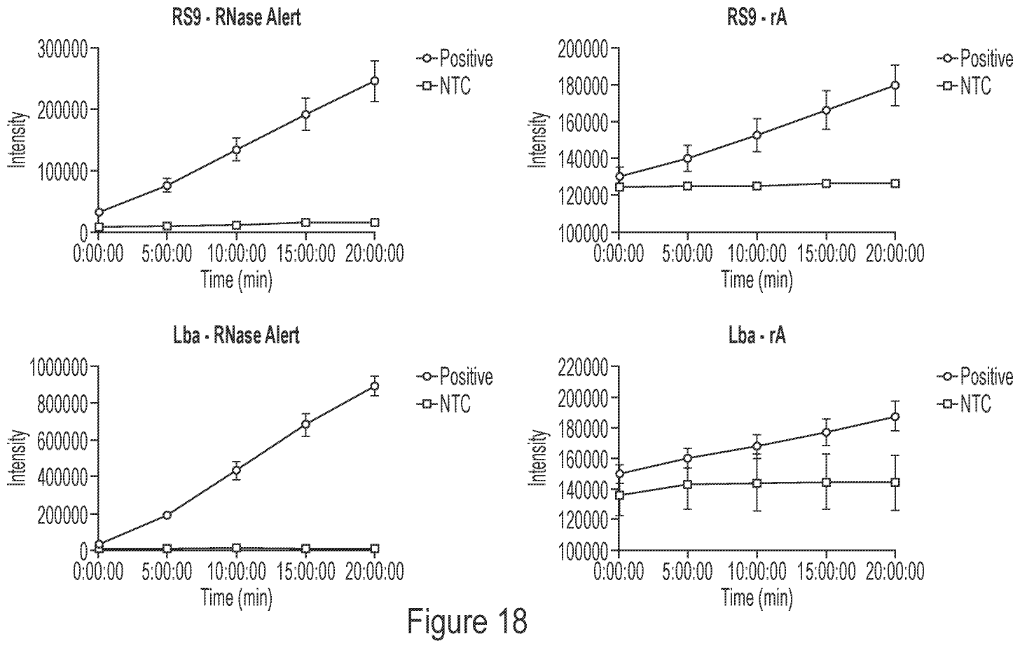

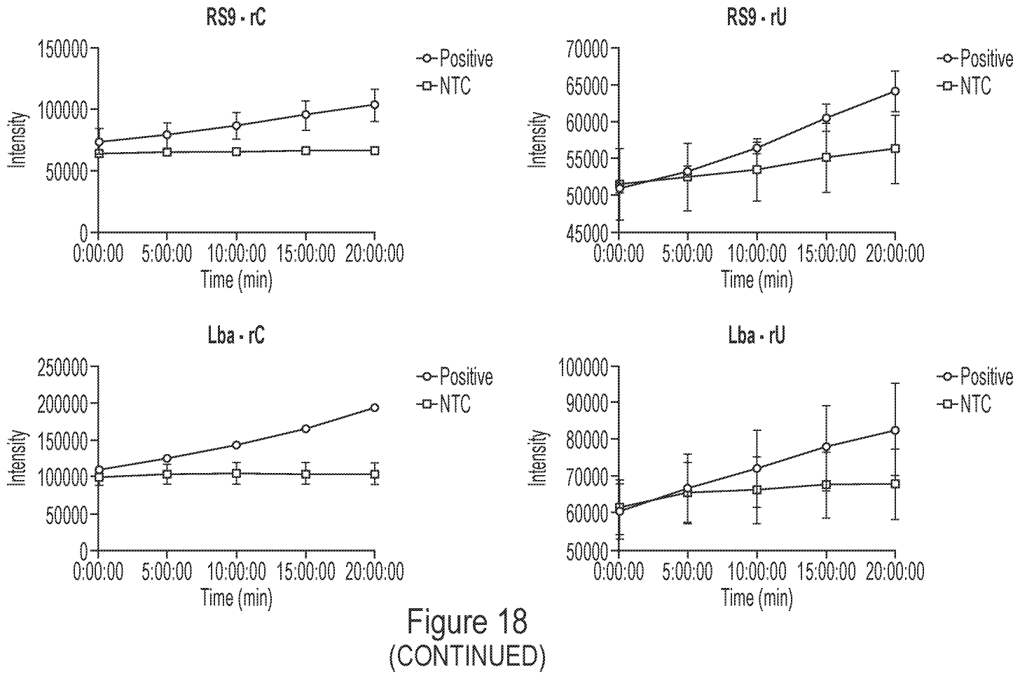

RS9 collateral cleavage activity was further evaluated alongside known Cas12a, LbaCas12a, using ORF LAMP product as target and either RNaseAlert, PolyrA, PolyrC, or PolyrU reporters. A no target condition was utilized as a negative control. Both RS9 and LbaCas12a were able to cleave RNaseAlert more efficiently than either PolyrA, PolyrC, or PolyrU (FIG. 18).

### Example 7: Exemplary Characterization of a Thermostable Cas13a

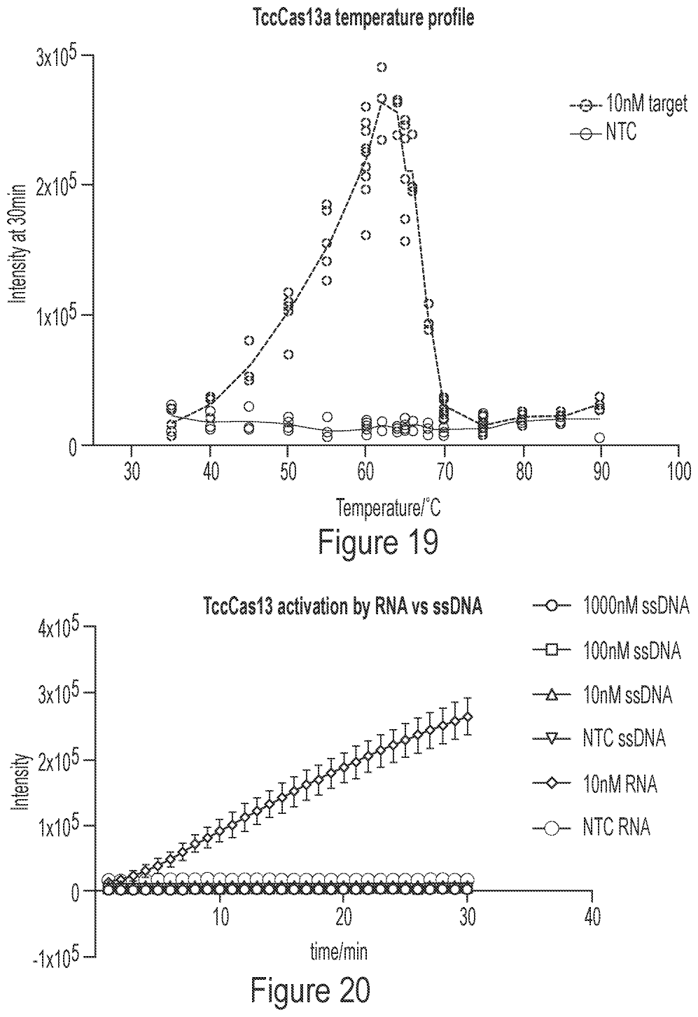

The present example demonstrates characterization of an exemplary thermostable Cas13a enzyme, TccCas13a. To determine optimal temperature for TccCas13a activity, a Cas reaction was conducted over a range of temperatures using 10 nM target and RNaseAlert as reporter. A no target condition was utilized as a negative control. Temperature profiles suggest TccCas13a shows highest activity at approximately 62° C. (FIG. 19).

To determine whether TccCas13a could be activated by ssDNA in addition to RNA, a Cas reaction was completed at 62° C. with RNaseAlert as a reporter. Different targets were utilized at different concentrations (e.g., 10 nM, 100 nM, or 1,000 nM ssDNA or 10 nM RNA). A no target condition was utilized as a negative control. Results indicated that TccCas13a can be activated by RNA, but cannot be activated by ssDNA even at the highest concentrations of ssDNA template at 62° C. (FIG. 20).

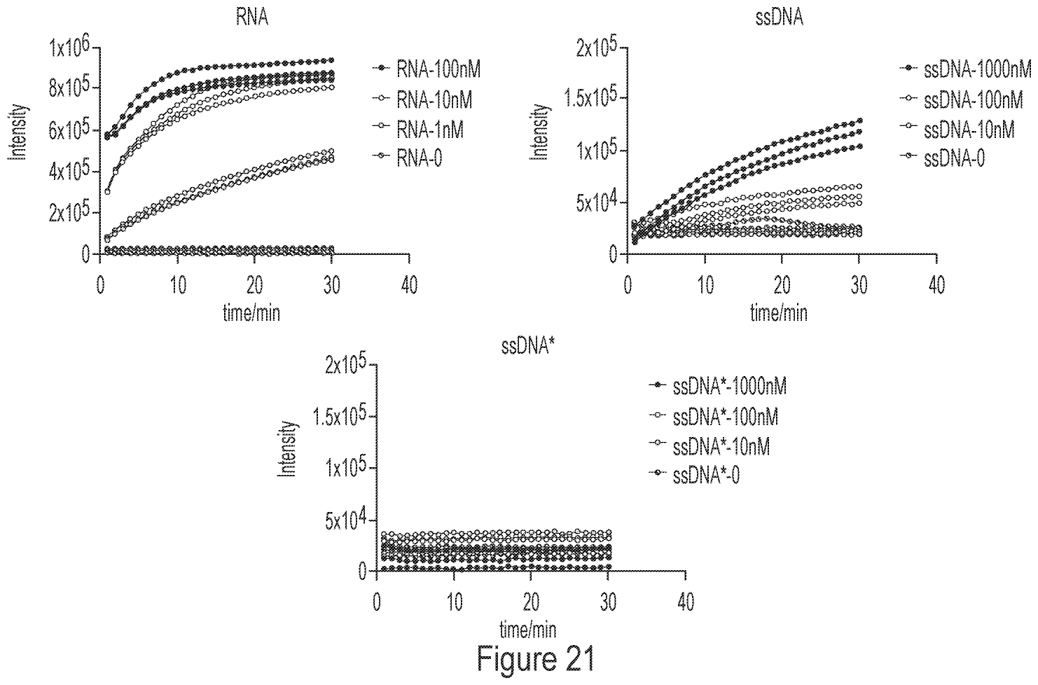

TccCas13a activation by ssDNA was also assessed at 58° C. TccCas13a was activated at 58° C. in the presence of 1 nM, 10 nM, and 100 nM RNA target compared to no target control, while TccCas13a activation by ssDNA at 58° C. was only detected when 100 nM or 1,000 nM of ssDNA target was utilized. 10 nM of ssDNA target at 58° C. showed no difference compared to no target control, suggesting TccCas13a activation requires a higher concentration of ssDNA than RNA, similar to that observed for LwaCas13 ssDNA activation. Interestingly, TccCas13a was not activated by ssDNA* at any concentration (10 nM, 100 nM, or 1,000 nM) compared to control (no target) (FIG. 21).

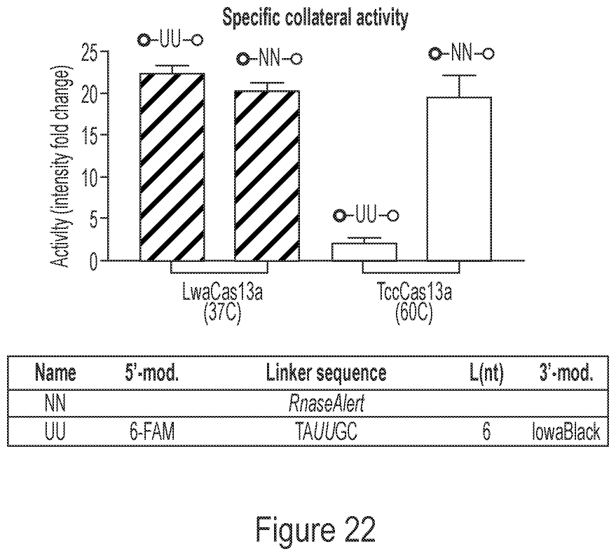

To determine whether TccCas13a showed different specific collateral activity to that of LwaCas13, a Cas reaction was done with two different reporters, RNaseAlert which contains multiple different bases (“NN”) and a “UU”-specific reporter with only two “UU” bases and a DNA backbone. Reactions were conducted at 60° C. LwaCas13a showed no preference for collateral activity of either reporter over the other, while TccCas13a showed increased collateral activity at “NN” sites compared to “UU” sites (FIG. 22).

### Example 8: Exemplary Characterization of Additional Candidate Thermostable Cas Enzymes

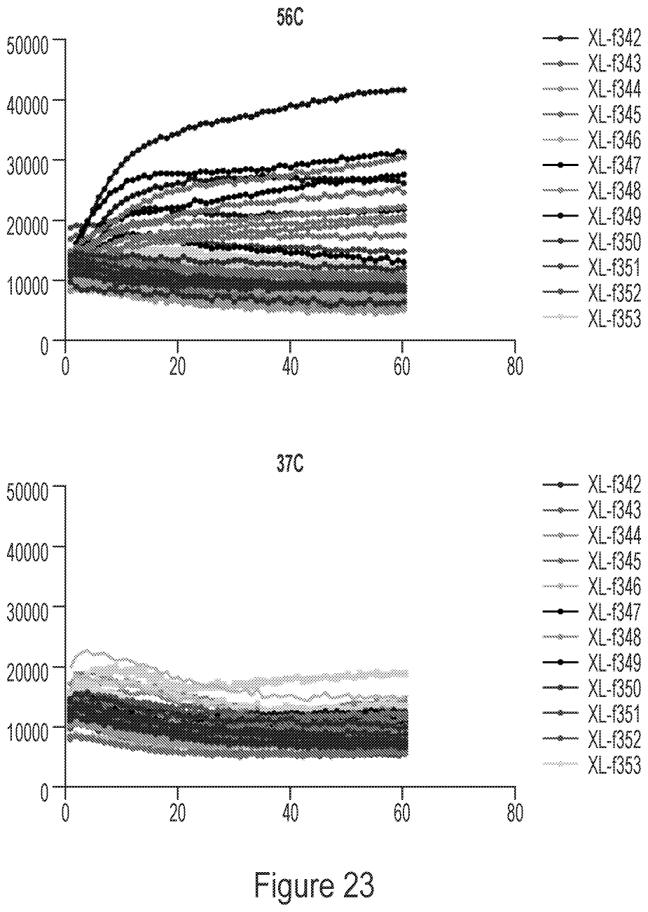

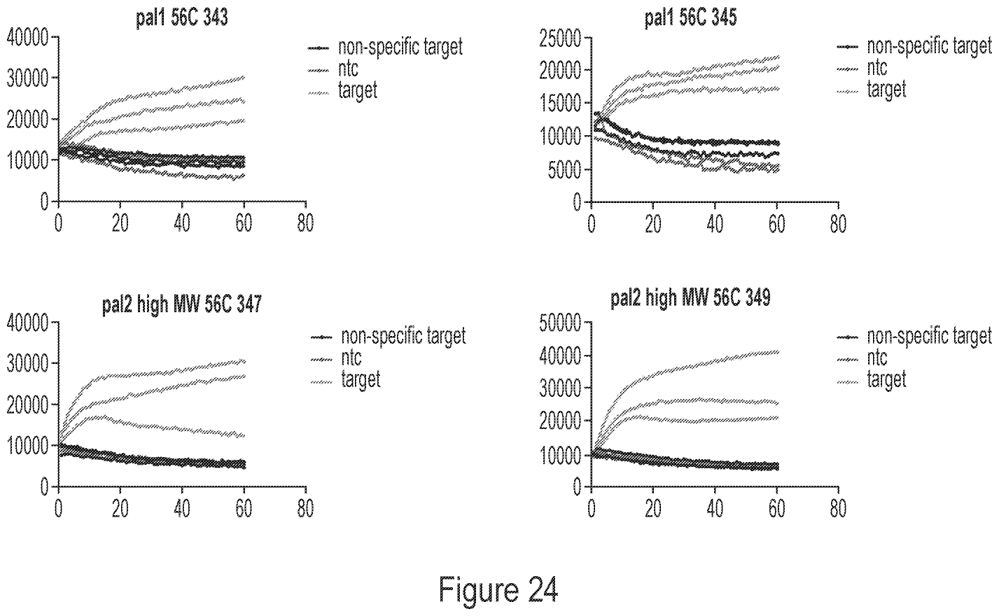

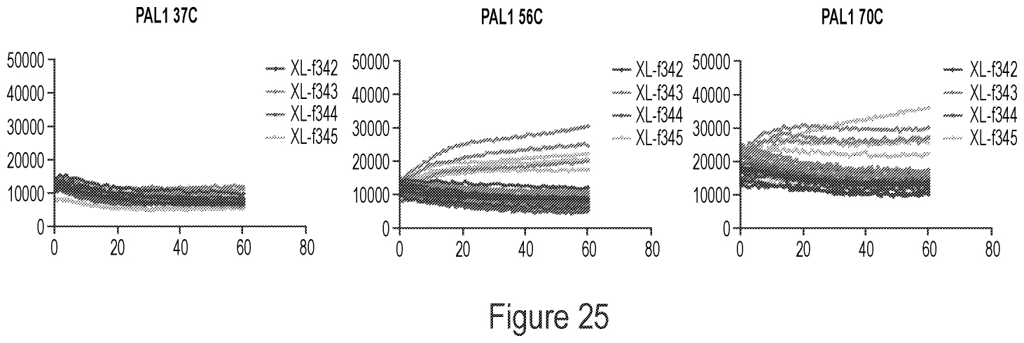

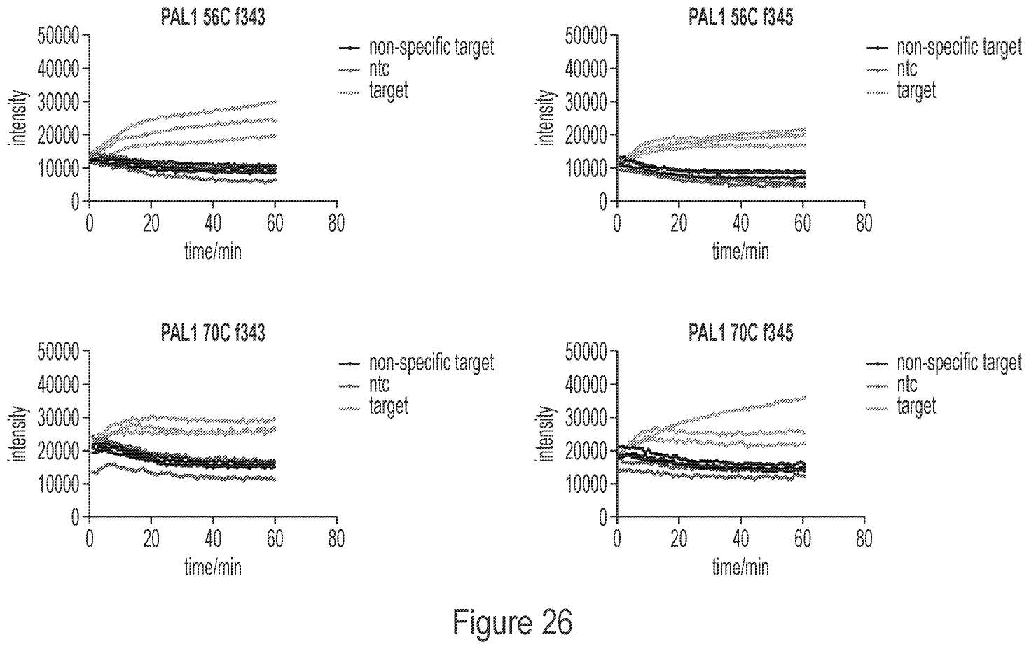

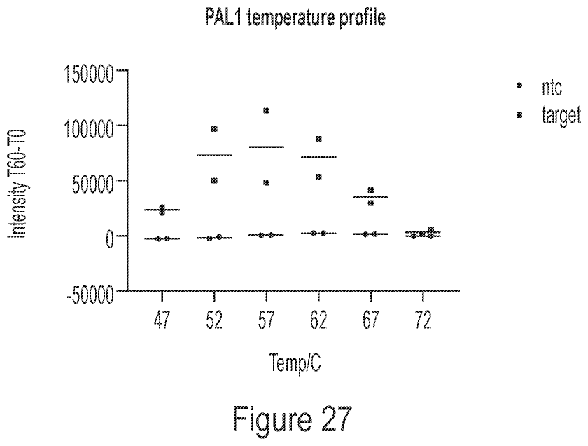

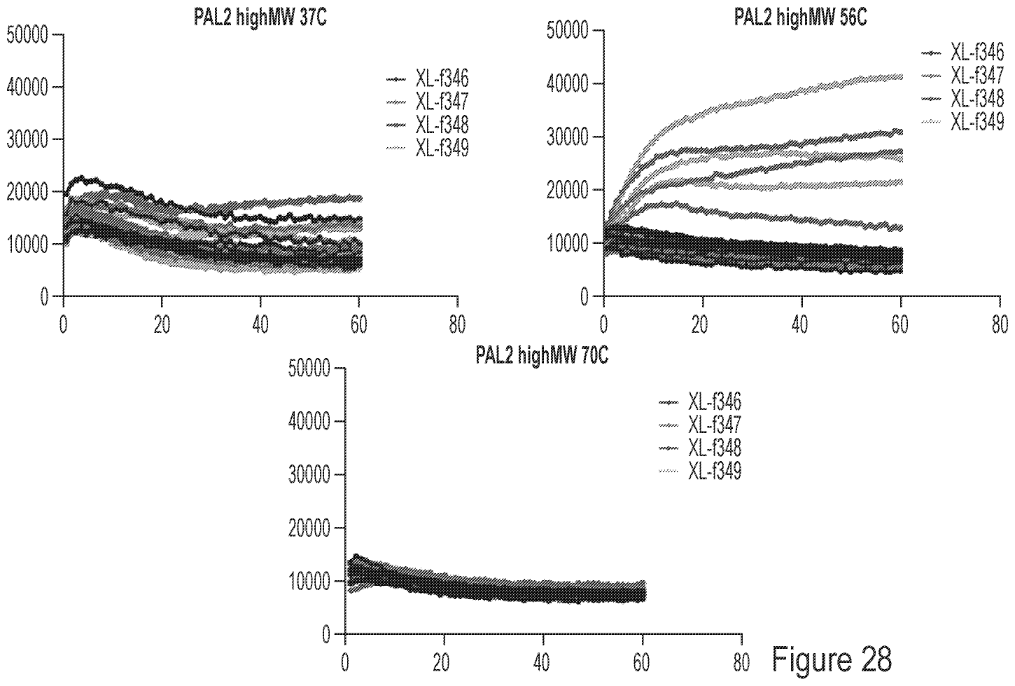

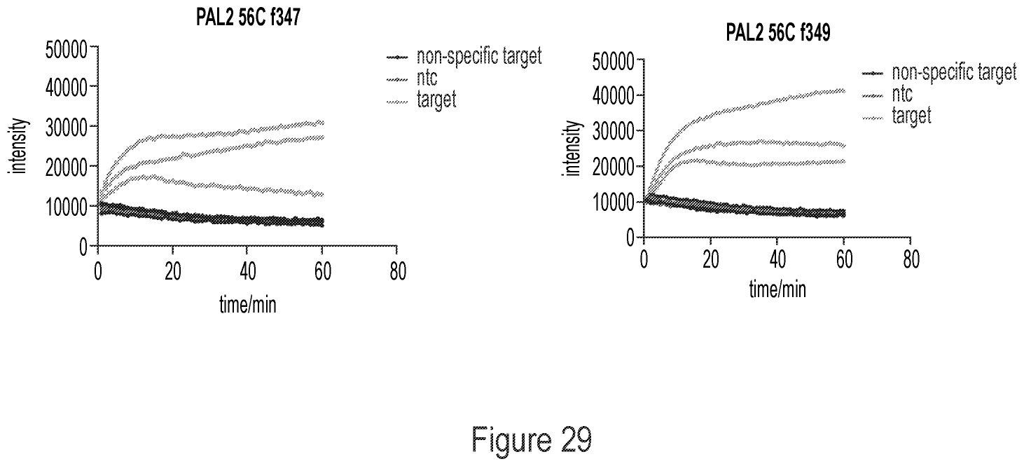

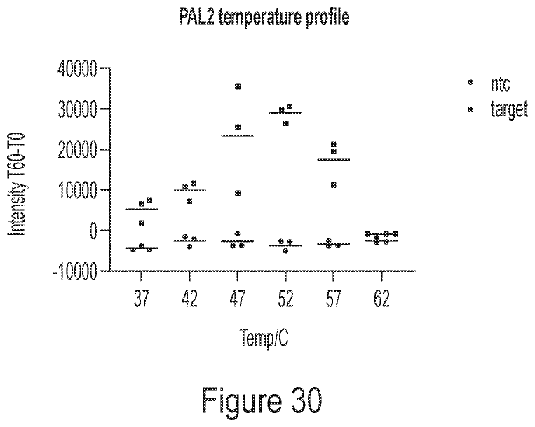

The present example demonstrates characterization of exemplary thermostable Cas enzymes, Pal1 (SEQ ID NO. 274), Pal2 low MW, Pal2 high MW (SEQ ID NO. 275), and Pal3 (SEQ ID NO. 276). Each enzyme was tested with four guides (designated as 342-353) at both 37° C. and 56° C. in a Cas-only reaction with DnaseAlert as a reporter. Fluorescence signal was plotted vs. time for each reaction (FIG. 23). Pal1 showed low activity for two guides at 56° C. No activity was observed for Pal2 low MW or Pal3, while activity was observed for two guides at 56° C. for Pal2 high MW. Pal1 and Pal2 activity at 56° C. is shown in FIG. 24. Additional results of studies with these enzymes are shown in FIGS. 25-30. As can be seen, Pal1 showed activity for 2 guides at 56° C. and 70° C.; Pal1 showed maximum activity at 57° C. and significant activity to at least 67° C. Pal2 high MW showed activity for 2 guides at 56° C.; Pal2 high MW also showed maximum activity at 47-52° C. and significant activity up to at least 57° C. No significant activity was observed for Pal2 low MW, or any of Pal3-6 at 37° C., 56° C., or 70° C. Those skilled in the art thus understand that these enzymes are thermostable at least at about 56° C. and/or within a range of 56° C. and 70° C. These particular exemplified enzymes may also be described as thermoactive as their relevant activity(ies) are dramatically reduced and/or undetected at lower temperatures such as at 37° C. Without wishing to be bound by any particular theory, it is noted that enzymes of thermophilic organisms often may show reduced (or undetectable) activity at such temperatures.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A detection method comprising steps of:
contacting a CRISPR-Cas complex comprising:
a Cas protein with collateral cleavage activity that is thermostable above a temperature of about 55° C.; and
a guide RNA selected or engineered to be complementary to a target sequence; with
a sample potentially comprising a nucleic acid of the target sequence.

2. The method of claim 1, wherein the step of contacting comprises contacting the CRISRP-Cas complex and sample with a reporter susceptible to cleavage by the Cas protein collateral activity.

3. The method of claim 1, wherein the step of contacting comprises incubating for a period of time above the temperature.

4. The method of claim 1, further comprising a step of amplifying nucleic acid present in the sample.

5. The method of claim 4, wherein the step of amplifying utilizes a thermostable nucleic acid polymerase.

6. The method of claim 4, wherein the steps of amplifying and contacting are performed in a single vessel.

7. The method of claim 1, wherein the Cas protein is a Cas12 protein.

8. The method of claim 7, wherein the Cas protein has an amino acid sequence that is at least 80% identical to that of SEQ ID NO: 15.

9. The method of claim 7, wherein the Cas protein has an amino acid sequence having at least 80%, sequence identity to any one of SEQ ID Nos. 3-21, 33-47, 51-56, 68-178, and 274-283.

10. The method of claim 1, wherein the Cas protein has an amino acid sequence having 80%, sequence identity to any one of SEQ ID Nos. 1-283.

11. In a method of performing a detection assay utilizing a Cas protein with collateral cleavage activity, the improvement that comprises utilizing a Cas protein with thermostable collateral cleavage activity.

12. The improvement of claim 11, wherein the Cas protein is a Cas12 protein.

13. The improvement of claim 12, wherein the Cas protein has an amino acid sequence that is at least 80% identical to that of SEQ ID NO: 15.

14. The improvement of claim 12, wherein the Cas protein has an amino acid sequence having at least 80%, sequence identity to any one of SEQ ID Nos. 3-21, 33-47, 51-56, 68-178, and 274-283.

15. The improvement of claim 11, wherein a method of performing a detection assay is conducted in a single reaction vessel.

16. The improvement of claim 11, wherein the thermostable collateral cleavage activity is thermostable above a temperature of about 55° C.

17. The improvement of claim 11, wherein the thermostable collateral cleavage activity is thermostable above a temperature of about 5Q° C.

18. The improvement of claim 11, wherein the Cas protein has an amino acid sequence having at least 80% sequence identity to any one of SEQ ID Nos. 1-283.


SEQUENCE LISTING (294 sequences)

SEQ ID NO: 1 - protein, 1225 aa, Thermoclostridium caenicola
MKITKRKWGEHHPPLYFYRDEDSGRLLAQNDRKQDYTDTLFNDIAQDTFERSLRNRLLKTPEKGDKRFYSNEIVKLVEKLCQGADVAEIMKSMERNEKLRPKNEKEIKNLKKQLDGTLSEYGKRYTAPEGAMTLNDALFYLVEGNPLKQAMAKAELGKIREALIKEKENRINRVRYSIKNNKIPLRIQEDGGITPNNDRAAWLLGLMKPADPAKGITDCYPLLGELEEVFDFDKLSKTLHEKISRCQGRPRSIAMAVDEALKQYLRELWEKSPSRQQDLKYYFQAVQEYFKDNFPIRTKRMGARLRQELLKDKTSLSRLLEPKHMANAVRRRLINQSTQMHILYGKLYAYCCGEDGRLLVNSETLQRIQVHEAVKKQAMTAVLWSISRLRYFYQFEDGDILSNKNPIKDFRDKFLRDTNKYTHEDVEACKEKLQDFFPLKELQEKIKEDAKGLQETDNKQADTTDFKAIGHIVRDDRKLCNQLLAECVSCIGELRHHIFHYKNVTLIQALKRIADKVKPEDLSVLRAIYLLDRRNLKKAFAKRISSMNLPLYYREDLLSRIFKKEGTAFFLYSAKIQMTPSFQRVYERGKNLRREFECERMKAEASNGQNGQDGDRLKWFRQLAAGDSADTHFNWAVEAYAESAADVENNVEFDTDVDAQRALRNLLLLIYRHHFLPEVQKDETLVTGKIHKVLERNRQLSEGQGPNQGKAHGYSVIEELYHEGMPLSDLMKQLQRRISETERESRELAQEKTDYAQRFILDIFAEAFNDFLEAHYGEEYLEIMSPRKDAEAAKKWVKESKTVDLKTSIDEKEPEGHLLVLYPVLRLLDERELGELQQQMIRYRTSLASWQGESNFSEEIRIAGQIEELTELVKLTEPEPQFAEEVWGKRAKEAFEDFIEGNMKNYEAFYLQSDNNTPVYRRNMSRLLRSGLMGVYQKVLASHKQALKRDYLLWSEKHWNVKDENGADISSAEQAQCLLQRLHRKYAESPSRFTEEDCKLYEKVLRRLEDYNQAVKNLSFSSLYEICVLNLEILSRWVGFVQDWERDMYFLLLAWVRQGKLDGIKEEDVRDIFSEGNIIRNLVDTLKGENMNAFESVYFPENKGSKYLGVRNDVAHLDLMRKNGWRLEAGKTCSVMEDYINRLRFLLSYDQKRMNAVTKTLQQIFDRHKVKIRFTVEKGGMLKIEDVTADKIVHLKGSRLSGIEIPSHGERFIDTLKALMVYPRG

SEQ ID NO: 2 - protein, 1217 aa, Thalassospira profundimaris
MRIIKPYGRSHVEGVATEQPRRKLRLNTRPDISRDIPGFAQSHDALIIAQWISAIDKIATKPKPDQKPTQRQMNLRTTLGDAAWQHLMAKNLLPAAKDPAIREKLHLIWQSKIAPWGASRPQEEKRGKPTPKGGWYERFCGALSPEAITQNVARQIAKDIYDHLYVAAKRKGREPVKQGESSNKPGKFKPDRKLSLIEERAESIAKNALRPGTHAPCPWGQDDQAIYEQAGDVATKIYDDARDYLEDKKRRSGNRNTSSVQYLPRDLAVKILYAQYGRVFGPDTTIKAALDEQQSLFALHTAIKDCYHRLVNDARKRHILRILPRNMAALFRLVRAQYDNRDINALIRLGKVIHYHAGEQGKDEHHGIRDYWPSQQDIQNSRFWGSDGQADIKRHEAFSRIWRHIIALASRTLHDWADPDSQKFTGDDDDILMRAGAIESNVWDAGRYERKCDVLFGAQASLFCGAEDFEKATLKQAITGTGNLRNATFHFKGKARFENELQRLADDVPVDVQSAIAALWQKDAEGRTRQIAETLQAVLAGHFLSERQNRHILATLMAAMAQPGDVPLPRLRRVLARHDSICQRGRILPLPPCPDRAKLEESPALTCQYTVLKMLYDGPFRAWLAQQNSTILNHYIDSTIARTNKAAQDMNGRKLAPAEKDLITARAADIPRLSVDEKMVDFLGRLTAATATEMRVQRGYQSDGEKAQKQAGYIGEFECDVIARAFSDFLGQSGFDFVLKLKADTPKPDAAQCDVAALIAPGDVPALTPQAWQQVLYFILHLVPVDDASRLLHQTRKWQALEKKGKDKEVKKEKDKEVKKEDEKPDIADLQSVLMLYLDMHDAKFTGGAALHGIEKFAEFFVEKADFRAVFPPQSLQDQDRSIPRRGLREIVRFGHLPLLQHMSGTVKITHDNVVAWQTARTPDATGTSPIARRQKQREELHALAVERPARFRNADLHNYMHALVDVIKHRQLSAQVTLSDQVRLHRLMMGVLGRLVDYAGLWERDLYFVLLALLYHHGVTPDDVLKGQGKRKLADGQVVEALKPKNRKAAAPVGVFDDLDHYGIYQDDRQSIRNGLSHFNMLRGGTAPDLSHWVNQTRRLVAHDRKLKNAVAKSVIEMLAREGFDLDWTIEPDSGKHILRHGKIRTRQAQHFQKSRIRIEKKSAKPDKNDTVKIRENLHGDAMVERVARLFAARAQKYRDITTEKRLDHLFLKPKG

SEQ ID NO: 3 - protein, 1129 aa, Alicyclobacillus acidoterrestris
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 4 - protein, 1147 aa, Alicyclobacillus kakegawensis
MAVKSIKVKLRLSECPDILAGMWQLHRATNAGVRYYTEWVSLMRQEILYSRGPDGGQQCYMTAEDCQRELLRRLRNRQLHNGRQDQPGTDADLLAISRRLYEILVLQSIGKRGDAQQIASSFLSPLVDPNSKGGRGEAKSGRKPAWQKMRDQGDPRWVAAREKYEQRKAVDPSKEILNSLDALGLRPLFAVFTETYRSGVDWKPLGKSQGVRTWDRDMFQQALERLMSWESWNRRVGEEYARLFQQKMKFEQEHFAEQSHLVKLARALEADMRAASQGFEAKRGTAHQITRRALRGADRVFEIWKSIPEEALFSQYDEVIRQVQAEKRRDFGSHDLFAKLAEPKYQPLWRADETFLTRYALYNGVLRDLEKARQFATFTLPDACVNPIWTRFESSQGSNLHKYEFLFDHLGPGRHAVRFQRLLVVESEGAKERDSVVVPVAPSGQLDKLVLREEEKSSVALHLHDTARPDGFMAEWAGAKLQYERSTLARKARRDKQGMRSWRRQPSMLMSAAQMLEDAKQAGDVYLNISVRVKSPSEVRGQRRPPYAALFRIDDKQRRVTVNYNKLSAYLEEHPDKQIPGAPGLLSGLRVMSVDLGLRTSASISVFRVAKKEEVEALGDGRPPHYYPIHGTDDLVAVHERSHLIQMPGETETKQLRKLREERQAVLRPLFAQLALLRLLVRCGAADERIRTRSWQRLTKQGREFTKRLTPSWREALELELTRLEAYCGRVPDDEWSRIVDRTVIALWRRMGKQVRDWRKQVKSGAKVKVKGYQLDVVGGNSLAQIDYLEQQYKFLRRWSFFARASGLVVRADRESHFAVALRQHIENAKRDRLKKLADRILMEALGYVYEASGPREGQWTAQHPPCQLIILEELSAYRFSDDRPPSENSKLMAWGHRGILEELVNQAQVHDVLVGTVYAAFSSRFDARTGAPGVRCRRVPARFVGATVDDSLPLWLTEFLDKHRLDKNLLRPDDVIPTGEGEFLVSPCGEEAARVRQVHADINAAQNLQRRLWQNFDITELRLRCDVKMGGEGTVLVPRVNNARAKQLFGKKVLVSQDGVTFFERSQTGGKPHSEKQTDLTDKELELIAEADEARAKSVVLFRDPSGHIGKGHWIRQREFWSLVKQRIESHTAERIRVRGVGSSLD

SEQ ID NO: 5 - protein, 1108 aa, Caldibacillus hisashii
  source 1..1108: note = Bacillus hisashii
MATRSFILKIEPNEEVKKGLWKTHEVLNHGIAYYMNILKLIRQEAIYEHHEQDPKNPKKVSKAEIQAELWDFVLKMQKCNSFTHEVDKDEVFNILRELYEELVPSSVEKKGEANQLSNKFLYPLVDPNSQSGKGTASSGRKPRWYNLKIAGDPSWEEEKKKWEEDKKKDPLAKILGKLAEYGLIPLFIPYTDSNEPIVKEIKWMEKSRNQSVRRLDKDMFIQALERFLSWESWNLKVKEEYEKVEKEYKTLEERIKEDIQALKALEQYEKERQEQLLRDTLNTNEYRLSKRGLRGWREIIQKWLKMDENEPSEKYLEVFKDYQRKHPREAGDYSVYEFLSKKENHFIWRNHPEYPYLYATFCEIDKKKKDAKQQATFTLADPINHPLWVRFEERSGSNLNKYRILTEQLHTEKLKKKLTVQLDRLIYPTESGGWEEKGKVDIVLLPSRQFYNQIFLDIEEKGKHAFTYKDESIKFPLKGTLGGARVQFDRDHLRRYPHKVESGNVGRIYFNMTVNIEPTESPVSKSLKIHRDDFPKVVNFKPKELTEWIKDSKGKKLKSGIESLEIGLRVMSIDLGQRQAAAASIFEVVDQKPDIEGKLFFPIKGTELYAVHRASFNIKLPGETLVKSREVLRKAREDNLKLMNQKLNFLRNVLHFQQFEDITEREKRVTKWISRQENSDVPLVYQDELIQIRELMYKPYKDWVAFLKQLHKRLEVEIGKEVKHWRKSLSDGRKGLYGISLKNIDEIDRTRKFLLRWSLRPTEPGEVRRLEPGQRFAIDQLNHLNALKEDRLKKMANTIIMHALGYCYDVRKKKWQAKNPACQIILFEDLSNYNPYEERSRFENSKLMKWSRREIPRQVALQGEIYGLQVGEVGAQFSSRFHAKTGSPGIRCSVVTKEKLQDNRFFKNLQREGRLTLDKIAVLKEGDLYPDKGGEKFISLSKDRKCVTTHADINAAQNLQKRFWTRTHGFYKVYCKAYQVDGQTVYIPESKDQKQKIIEEFGEGYFILKDGVYEWVNAGKLKIKKGSSKQSSSELVDSDILKDSFDLASELKGEKLMLYRDPSGNVFPSDKWMAAGVFFGKLERILISKLTNQYSISTIEDDSSKQSM

SEQ ID NO: 6 - protein, 1090 aa, Laceyella sediminis
MSIRSFKLKIKTKSGVNAEELRRGLWRTHQLINDGIAYYMNWLVLLRQEDLFIRNEETNEIEKRSKEEIQGELLERVHKQQQRNQWSGEVDDQTLLQTLRHLYEEIVPSVIGKSGNASLKARFFLGPLVDPNNKTTKDVSKSGPTPKWKKMKDAGDPNWVQEYEKYMAERQTLVRLEEMGLIPLFPMYTDEVGDIHWLPQASGYTRTWDRDMFQQAIERLLSWESWNRRVRERRAQFEKKTHDFASRFSESDVQWMNKLREYEAQQEKSLEENAFAPNEPYALTKKALRGWERVYHSWMRLDSAASEEAYWQEVATCQTAMRGEFGDPAIYQFLAQKENHDIWRGYPERVIDFAELNHLQRELRRAKEDATFTLPDSVDHPLWVRYEAPGGTNIHGYDLVQDTKRNLTLILDKFILPDENGSWHEVKKVPFSLAKSKQFHRQVWLQEEQKQKKREVVFYDYSTNLPHLGTLAGAKLQWDRNFLNKRTQQQIEETGEIGKVFFNISVDVRPAVEVKNGRLQNGLGKALTVLTHPDGTKIVTGWKAEQLEKWVGESGRVSSLGLDSLSEGLRVMSIDLGQRTSATVSVFEITKEAPDNPYKFFYQLEGTELFAVHQRSFLLALPGENPPQKIKQMREIRWKERNRIKQQVDQLSAILRLHKKVNEDERIQAIDKLLQKVASWQLNEEIATAWNQALSQLYSKAKENDLQWNQAIKNAHHQLEPVVGKQISLWRKDLSTGRQGIAGLSLWSIEELEATKKLLTRWSKRSREPGVVKRIERFETFAKQIQHHINQVKENRLKQLANLIVMTALGYKYDQEQKKWIEVYPACQVVLFENLRSYRFSYERSRRENKKLMEWSHRSIPKLVQMQGELFGLQVADVYAAYSSRYHGRTGAPGIRCHALTEADLRNETNIIHELIEAGFIKEEHRPYLQQGDLVPWSGGELFATLQKPYDNPRILTLHADINAAQNIQKRFWHPSMWFRVNCESVMEGEIVTYVPKNKTVHKKQGKTFRFVKVEGSDVYEWAKWSKNRNKNTFSSITERKPPSSMILFRDPSGTFFKEQEWVEQKTFWGKVQSMIQAYMKKTIVQRMEE

SEQ ID NO: 7 - protein, 1133 aa, unidentified
  source 1..1133: note = Description of Unknown: Cas12 sequence
MFKKKLFDDEEFISLAQNQEESNALNAFKGFTTHFKDFQENRKNMYSEDKESTAIAYRIIHENLPVFITNNIRFEKIINELDRSNIHSIEKELKEELANNKLKDIFNIEYFQNTLTQNDITRYNTIIGGKVKADGKKVQGLNEYINLFNQHNKDKKLPLLKPLYKQILSEENSASFIVPAFEKDNEVLQSIFDFWNKCIIDAKGPISGKKYNLLSKIQSLLQNLDKLKNNQLEEMYFENENLSTISNDVYGQWNLIRDALGNFYNSIDAKKNKKDYYSWKEIQDALVYYKQTNDEYKDIDQKAFLIYFKEMKVNDGEENTNNNIINLINERYKRIEPLLKEDRDNRKDLHQDKGKVAIIKEFLDSLKLLQNTIKLLYVDDSLDNMNYDFYNQLTDYYETLRPLNTLYNRVRNYMTRKPFSEEKFVLTFNSPTLLDGWDLNKEEANLGVILRKDNKYYLGIMNKGDNKIFKKYDEEPGDDYYEKMVYKLLPGPNRMLRKVFFSNKNIEYYKPNQDIQNLYNKGEFKKGESLNKESLHKLIDFYKNSISKNGDWSVFNFKFKKTTAYDDISQFYKDVENQGYKLFFKTIKTSYIDQLVNEGKLYLFQIYNKDFSENKKRKDESNPNLHTIYFKNLFSEDNLKNVVYKLNGKAEVFYRKKSIEYPEEIRRKGHHYNELKDKFDYPIIKDKRYSEDKFLFHVPITLNFLAKSDEKVNEMVKNYIAATNEKIHIIGIDRGERNLLYLSLIDSNGNIVKQQSLNIIELPKYQKQIDYHAKLNEKEKQRLAARQNWDVIENIKELKEGYLSQVIHQIARLMVDYKAILVMEDLNFGFKRGRFKVEKQVYQKFEKMLIDKLSYLVFKEKNLCEPGGSLRAYQLSAPFKSFKALGKQSGMIFYVPAQYTSKIDPTTGFYNFLNIDVSNLARSKETFSKFDKIVYNKKEDYFEFYCKMINFESANQLTKKSQNKANAELKEFQWILCSTHHDRFKVERKNNQINYCKINVNEELKKLLNSKGINYEKSNDLKSEILNIDESKFFKELGYLLKILVSLRYNNGKKGSEEQDFILSPVKNASGKFFCTLDNNNTLPLDADANGAYNIALKGLMIVQRVKAGGKLDLSISKDDWINFLIMNKKLPK

SEQ ID NO: 8 - protein, 1352 aa, unidentified
  source 1..1352: note = Description of Unknown: Cas12 sequence
MSNQSVFKDFTNLYELSKTLRFELKPVGKTLRMLEDAKVFKTDELIQKKYEQTKPFINKLHQEFVKESLEGRSLEGLESYQDILKEWQKDKKDKIAQKNLGIKEKELYKQVTQLFNAKAKEWSEPYAHLGLKKKDIGILFEEGVFKILKEKYNNDKDAKITNKVTGEIFFEDFWKGFVGYFQKFFETRKNFYKDDGTSTAIATRIVAQNLKRFCDNIGLFEKIKDQIDSSEVEQSFGISMEKVFSLDFYNQCLLQGGIDKYNEILGGKTLENGEKFKGINELINKYRQDNKGDKSSFLKILDKQILSEKESFIDEIKNDKELEETLKNLHETAKVKTKIFGTLFEDFIGNNTKYDLAKIYISKEAFNTISHKWTGGTDLFAENLFNALKDEQILKSSAKKKDGSYVFPDFIEFLHIKTALENVPKDINFWKERYYVNKEGENKEFFLGNGEIWQQFLQIFNFEFNELFQKEIIDNQTGKKMHIGYKVYKEEISKLLEDFKVDKDSTVIIKHFADSVLWIYQMAKYFALEKKRTWRDEYDLDTFYTDPKNGYLAFYENAYEEIVQIYNKLRNYLTKKPYSTEKWKLNFQNSTLASGWDKNKEADNFTVILRKDGKYFLGLMRKGANKLFDKRYGSEFSQGLEKGKYEKMNYKYFPSPSKMIPKTSTQVHEVKKHFKNSSEPFFLEESSSLGKFIKQLKITKEVFDLNNFEYKKSYLSTLNGESPDESQRVKADSKKTGQVKLFQKEFLNLSQNELLYKKSLFAWVDFCKEYLDCFPSTGDGFLQFKKYIQDTEKYESIDQFYKDIERGGYKISFQNISEEYISCKNQNSELYLFKIHNKDWNLKDGKPKTGMKNLHTMYFESLFSSENIAQNFPMKLNGQAEIFYRPKTDINKLEMKKDSKGKNVVDHKRYEEDKIFFHLPMTLNRGKSLFNFNVQLNNFLADNPEINIIGVDRGEKHLAYYSVINQNQEILDGGTLNVVKGGNGKDIDYHKKLEDKAEKREQARKDWQDVEGIKDLKKGYISQVVRKLADLAIEHNAIIVFEDLNMRFKQIRGGIEKSVYQQLEKALIEKLSFLVRKNEKNPEEAGYLLKAYQLSAPFETFQRIGKQTGIIFYTQASYTSKIDPLTGWRPNLYLKYSNAKKAKADISKFSEIEFINNRFEFTYDLQEFRSQKDKKKEYPKKTLWTLCSSVERYRWNRKLNDNKGGYEHYSDLTSDFKKLFKKYNININEDILGQIENMDTDDRKNNARFFSGFMFFWNLICQIRNTNSDVISGESDNDFILSPVEPFFDSRKASQFGSDLPENGDDNGAFNIARKGIMILKKISQYVEENENCDKLKWGDLYISHTDWDNFI

SEQ ID NO: 9 - protein, 921 aa, unidentified
  source 1..921: note = Description of Unknown: Cas12 sequence
MTNYTDFIGLYPVQKTLRFELRPQGKTAEKMRESGLLEQDREKAKNYIVMKALIDDYHRRFINELLEKASFDWQPLFEALNNVKVNKDDKSKKELEKEQLHMRKELIGLFEKDERFKYLFSEKLFSELLNKEISERNDPDEMEAMRSFDRFSGYFIGFHENRRNIYSNEDKHNSLAYRVVAENFPKFADNCRKYSLIKENMQEAVVEFKKEIASVVDIDVDQMFDISYFNKVLTQKGIDDYNTMLGGVSEEGSVKIRGLNEFLNLYYQKVTDNKRIKMAPLYKQILCESKTKSFIPYMFENDEEVISSINQYYDSVKYDILQRSVYLLSNYKEYDASKIFIDQKSISSISIVLFGSWETLGGLMQIYKADQIGDPGLEKTRKKVDKWLSSSYFTLKEVFEAIGEQDPFRVYVEKLSLVLKNIEEFDKSCLLEGTHFSGDELLTQDIKGFLDLLMEVQHLMKPFNAKEDLDKDAAFYSEYNEIYEALSEIIPLYNKVRNYATKKKYSTYKIKMNFGNPTLAAGWDLNKERDNTAVILLRGNNYYLGIMNPKKKTKFEELPSGEDNDCYRKMVYKLLPGPNKMLPKVFFSKKGIGTFNPSKEILEGYETGKHKLGDSFDIDYCHSLIDFFKENIPKYGDWGTYEFKFSPTEEYSDISQFYKEVSEQGYKITFQNISRKAIDDLVNNGALFLYQIYNKDFSEHSKGKNNLHTMYWKAAFSEENLRNVVIKINGEAELFYRDKSDISKTEHSAGTILVNRTDRKDNPIPNSIYYELFKYKTGQIKSVSDEAKQYLDDLVTHEAKYPITKDRRYTEDRMFFHIPITLNFGSSGNTNINKAVIDHVLNSKDVHIIGIDRGERNLLYVSVIDRKGNIIKQRSLNVIDGIDYHEKLDQREKENISARKSWSNVEKIKDLKEGYLSYVIH

SEQ ID NO: 10 - protein, 1238 aa, unidentified
  source 1..1238: note = Description of Unknown: Cas12 sequence
MKDFYQFTNLYALSKTLRFSLIPTPATKQMLEDAKVFEKDETIQKKYEATKPYFDRLHREFALEALQDQKLDFKNYLELYRKYKADKKASGKLLINIEKDLRKEVVKLFDKQGEKWAKQYPGLKNKNIGVLFKEAVFTVILKERYGNEKETQILDESSGQLVSIFDSWKGFIGYFKKFHETRKNFYKDDGTSTALATRIIDQNLKRFCDNILIFESTKEKVDFSEVEISFGKPLSEVFTLEFYNTCFLQNGIDFYTKILGGETLQNGEKVKGLNECINLHKQKTGEKLPFFKSLDKQILSEKDKFFIDEISNETQLLEVLKSFVASAESKTDTIKTLVDDFVKDQDKYDLNYIYFSNDGLNTITRKWTTETQVFEEALYTALKAAKVVSSSAKKNEGGYSFPDFIPFAHLKTALESIKIDGTIWRDNFNAIENFEEKSIWAQFLAIYNFELSNLFETEIKNPEIGNCPTIGYNVYKQDFEELLKSFVYDPNAKVTIKNFADNVLSIYQMAKYFAVEKKRGWNTDYELDVFYTDPQNGYLQYYENAYEEIVQVYNKLRNYLTKKPYSEEKWKLNFDSGTPIKYTTRAIIFNNTTNERYYLGLLKKGVAKPREFEPINNNIISSGEFRRMIIQQLKFQTLAGKGYVRDFGVKYSEDKDGVKHLQQLIKKQYLSKYPCLKKIADGVYNDKKAFDADIKDVLLETYNLDFQPISEEFILNKNRLGEIYLFEIHNKDWNLKDGKNKSGSKNLHTMYFESLFVDKTTFKLNNEGAEVFYRPATNEGKLGTKKDRNGKIIINHKRYATDKILFHCPIGLNKDAGKSYTFNAKINNMLANNPDINIIGVDRGEKHLAYYSVITQKGKILDRGSLNKVEGGDKQEIDYAKKLEETAKNREQARKDWQAVEGIKDLKRGYISQVVRKLADLAIEHNAIIVFEDLNMRFKQIRGGIEKSVYQQLEKALIDKLSFLVMKGEADPEKAGHLLKAYQLVAPFESFQSMGKQTGIIFYTQANYTSKIDPITGWRPNLYLKYTSAEKAKADILKFSKIEFVNNRFELTYDIKNFVLDKKVVLSNKTKWTVCSSVERFRWNRRLESNQGNYEHYENLTENLSSLFKDFGFEIEQNIIRQVEQLATKGNEQFFRSFIFYVNLIFQIRNTDAKAKDQNKEDFILSPVEPFFDSRTPEKFGENLPENGDDNGAFNIARKGIIMLNKISAYKQEVGNVDKIIWKDLFISAAEWDNFTQE

SEQ ID NO: 11 - protein, 1301 aa, unidentified
  source 1..1301: note = Description of Unknown: Cas12 sequence
MDSYEQFTKLYPIQKTIRFELKPQGRTKEHFDNSNFLEKDRERDDNYKILKEVIDDYHREFIDECLSNIQLNWDDLKKFSEEYRRSKEKKNNRDSESEQKRMSTTSETRAINKKNLEAEQKRMRGEIVSAFKKDDRFKHLFSEKLFSILLKNQIYEKGTLEEIEAFDCFNKFSGYFKSFHENRKNMYSDEDKETAISYRIINENFPKLLDNFEKYQYVCREYPEQIREAESTLAEAGCYIKMDEIFSIDNFNNVMMQGGKESGISRYNLAIGGIVQGTGEKPKGLNEFLNLAYQNEPNGRKKIRMEPLYKQILSKEESFSYRLEAFTDDSQLLSAIRSFFDIVEKDKNGNIFDRAVNLMSSFSNYDTSKIYIRKAYLNQVSKEIFGYRGKSDSKPAKTADESLNKSGGWEKLGQMLRDYKADSIGDRNLEKTCKKVDKWLDSDEFTLSDILGAISLAGSNETFEAYVSEICVARRNIDKEKEKEKNINVEKISGDTESIQIIKALLDSVQEFFHLLSPFQLHPNTPHDWTFYAEFNDIYDKLSAITPLYNQARNHLTKKNLDTSKIKLNFNNPTLANGWDVNKEYENTAVILIRDGKYYLGIMNPKNKRKIKFDEGSGAGPFYQKMVYKLLPGPYRMLPKVFFAKKNIDYYNPSQEIREGYKAGKHKKGKEFDKGFCHKLIDFFKESIQKNENWKVFDFKFSPTESYDDISEFYQEVEKQGYRMYFVNIPSDTIDRYVEGGDMFLFQIYNKDFAKGAKGNKDMHTLYWNAVFSEENLQKGVMKLSGEAELFYRKKSDIKDPPHREGEILVNRTYIDRTHVSGVMGEQNTVKESRIPVPDEIHKNLFDYYNHGRELTKEEKEYCDKVGSFKAYYGIVKDRRYLENKMYFHVPLTLNFKAIGEKRINKMAIEKFLTDENACIIGIDRGERNLLYYSIIDRNGKIIDQKSLNVIDGFDYHEKLSQRQTEREVARQSWNSIGKIKDLKEGYLAKAVHEISKMAIKYNAIVVLEDLHFGFKKGRLKVEKQIYQKFEEMLINKLNYLVFKDVSDSSDAGGVLNAYQLTAPLESFSKLGKQSGILFYVPAAFTSVIDPTTGFVDLFNSSSITSTQKKKEFLQRFESIVYSARDGGIFAFTFDYRNFSKIATDHRNMWTVYTHGERIRYVRDEKCYKTTDPTKRIKEALSGIEYDDGSDIRDKITQSGDNNLINTVYHSFMDTIKMRNKDGRIDYIISPVKNRNGEFFRSDYKHRDFPVDADANGAYHIALKGELLMRMIGKTYDSNSDKMPKLEHKDWFEFMQTRGDQ

SEQ ID NO: 12 - protein, 1368 aa, unidentified
  source 1..1368: note = Description of Unknown: Cas12 sequence
MKKEKEFKSFGDFTNLYEISKTLRFELKPVENTQTMLDEADVFGKDKVIKDKYTKTKPFIDKLHREFVDESLKDVSLSGLKKYSEVLENWKKNKKDKDIVKELKKEEERLRKEVVEFFDNTAKKWANEKYKELGLKKKDIGILFEESVFDLLKEKYGEEQDSFLKEEKGDFLKNEKGEKVSIFDEWKGFVGYFTKFQETRKNFYKNDGTETALATRIIDQNLKRFCDNIDDFKKIKNKIDFSEVEKNFNKTADVFSLDFYNQCLLQKGIDSYNEFIGGKTLENGKKLKGVNELVNEYRQKNKNEKVSFLKLLDKQILSEKEKLSFGIENDEQLLVVLNSFYETAEEKTKILRTLFGDFVEHNENYDLDKTYISKVAFNTISHKWTNETHKFEELLYGAMKEDKPIGLNYDKKEDSYKFPDFIALGYLKKCLNNLDCDTKFWKEKYYENNADKKDKDKGFLTGGQNAWDQFLQIFIFEFNQLFNSEAFDNKGKEIKIGYDNFRKDFEEIINQKDFKNDENLKIAIKNFADSVLWIYQMAKYFAIEKKRGWDDDFELSEFYTNPSNGYSLFYDRAYEEIVQKYNDLRNYLTKKPYKEDKWKLNFENPTLANGFDKNKESDNSTVILRKKRKYYLGLMKKGNNKIFEDRNKAEFIRNIESGAYEKMAYKYLPDVAKMIPKCSTQLNEAKNHFRNSADDLEIKKSFSNPLKITKRIFDLNNIQYDKTNVSKKISGDNKGIKIFQKEYYKISGDFDVYKSALNDWIDFCKDFLSKYDSTKDFDFSILRKTKDYKSLDEFYVDVAKITYKISFTPVSESYIDQKNKNGELYLFEIYNQDFAKGKMGAKNLHTLYFENVFSPENISKNFPIKLNGNAELFFRPKSIESKKEKRNFVREIVNKKRYSEDKIFFHCPITLNRETGSIYRFNNYVNNFLSENNINIIGVDRGEKHLAYYSVIDKNGVKIGGGSFNEINKVDYAKKLEERAGEREQSRKDWQVVEGIKDLKKGYISQVVRELADLAIKHNAIIVLEDLNMRFKQIRGGIEKSIYQQLEKALIDKLSFLVEKGEKDPNQAGHILKAYQLAAPFTSFKDMGKQTGIVFYTQASYTSKTCPNCGFRKNNNKFYFENNIGKAQDALKKLKTFEYDSENKCFGLSYCLSDFANKEEVEKNKNKKRNNAPYSDIEKKDCFELSTKDAVRYRWHDKNTERGKTFFEGESVYEEKEEKEIGQTKRGLVKEYDISKCLIGLFEKTGLDYKQNLLDKINSGKFDGTFYKNLFNYLNLLFEIRNSISGTEIDYISCPECQFHTDKSKTIKNGDDNGSYNIARKGMIILDKIKQFKKENGSLDKMGWGELFIDLEEWDKFAQKKNNNIIDK

SEQ ID NO: 13 - protein, 1285 aa, unidentified
  source 1..1285: note = Description of Unknown: Cas12 sequence
MKSFDSFTNLYSLSKTLKFEMRPVGNTQKMLDNAGVFEKDKLIQKKYGKTKPYFDRLHREFIEEALTGVELIGLDENFRTLVDWQKDKKNNVAMKAYENSLQRLRTEIGKIFNLKAEDWVKNKYPILGLKNKNTDILFEEAVFGILKARYGEEKDTFIEVEEIDKTGKSKINQISIFDSWKGFTGYFKKFFETRKNFYKNDGTSTAIATRIIDQNLKRFIDNLSIVESVRQKVDLAETEKSFSISLSQFFSIDFYNKCLLQDGIDYYNKIIGGETLKNGEKLIGLNELINQYRQNNKDQKIPFFKLLDKQILSEKILFLDEIKNDTELIEALSQFAKTAEEKTKIVKKLFADFVENNSKYDLAQIYISQEAFNTISNKWTSETETFAKYLFEAMKSGKLAKYEKKDNSYKFPDFIALSQMKSALLSISLEGHFWKEKYYKISKFQEKTNWEQFLAIFLYEFNSLFSDKINTKDGETKQVGYYLFAKDLHNLILSEQIDIPKDSKVTIKDFADSVLTIYQMAKYFAVEKKRAWLAEYELDSFYTQPDTGYLQFYDNAYEDIVQVYNKLRNYLTKKPYSEEKWKLNFENSTLANGWDKNKESDNSAVILQKGGKYYLGLITKGHNKIFDDRFQEKFIVGIEGGKYEKIVYKFFPDQAKMFPKVCFSAKGLEFFRPSEEILRIYNNAEFKKGETYSIDSMQKLIDFYKDCLTKYEGWACYTFRHLKPTEEYQNNIGEFFRDVAEDGYRIDFQGISDQYIHEKNEKGELHLFEIHNKDWNLDKARDGKSKTTQKNLHTLYFESLFSNDNVVQNFPIKLNGQAEIFYRPKTEKDKLESKKDKKGNKVIDHKRYSENKIFFHVPLTLNRTKNDSYRFNAQINNFLANNKDINIIGVDRGEKHLVYYSVITQASDILESGSLNELNGVNYAEKLGKKAENREQARRDWQDVQGIKDLKKGYISQVVRKLADLAIKHNAIIILEDLNMRFKQVRGGIEKSIYQQLEKALIDKLSFLVDKGEKNPEQAGHLLKAYQLSAPFETFQKMGKQTGIIFYTQASYTSKSDPVTGWRPHLYLKYFSAKKAKDDIAKFTKIEFVNDRFELTYDIKDFQQAKEYPNKTVWKVCSNVERFRWDKNLNQNKGGYTHYTNITENIQELFTKYGIDITKDLLTQISTIDEKQNTSFFRDFIFYFNLICQIRNTDDSEIAKKNGKDDFILSPVEPFFDSRKDNGNKLPENGDDNGAYNIARKGIVILNKISQYSEKNENCEKMKWGDLYVSNIDWDNFVTQANARH

SEQ ID NO: 14 - protein, 1366 aa, unidentified
  source 1..1366: note = Description of Unknown: Cas12 sequence
MNTQKKEFNPKSFKDFTNLYSLNKTLRFSLTPNKKTAEILEFNKQKEVKCFSNDRKIAGAYQEIKKYLNKLHQEFIQEAMKFFAFSEEELKGFEKEYLNLLNFTDKDNFKKKNKIRNEYEQERKILTIKIATYFSKFKSEKYQSFNLANITGKKVFSILEQKYKEDKKTLKIIHIFKYKPTKDEKKEGEAVNFSTYLTGFNENRKNFYKSEDKAGQFATRTIDNLAQFIKNKKLFEDKYQKNYSKIGILDEQIKIFNLDYFNNLFLQEGLDEYNGILGNNKGEENKSNEGINQKINIFKQKEKARLKKEKENFNKSDFPLFKELYKQIGSIRKENDVYVEIKTDKELVEELNNFPKNVENYLKDIQSFYKTFFEKLQNEEYELDKIYLPKSVGTYFSYIAFSDWNKLAFIYNKRYKNEKIKIVEGGDVNVQYRSLEVLKNRIDELKDEDNLNFNKFFIDKLKFNEAKKENNWQNFWFCIEYYINSQFIGGEKNILNKEKNEYEILPFGSLKELKEKYFEAVKKYKEKMVDTESGLTDDEEKEIKETLKNYLDRIKEIERIAKYFDLKKSFEEIKQEDLDSNFYGEYQKVVDKTNELKIYQYYSEFRNYLTQNNSVEEKIKLNFNSGLLLDGWDLNKEKVKFSIIFQENGKYYLGIINKEKDKTILDKDKHPEIFTKNSDFRKMEYKLFPSPSKMLPKISFSETAKKGDEDVGWSEEIQKIKDEFAEFQEYKKKSKDNWKDEFNRGKLNKLIDYYKQVLEKHSEGYMNTYNFELKDSSKYKNLGEFNDDIARQNYKVKFVGIDKNYIDEKVANGELFLFQIYNKDFSEDKKEGSTNNLETIYFKELFSKENLENPVFKLSGGAEMFFRNKIEKKKEKKKLDKDGKPMISKKGEKVVDKRRFSENKILFHLPIEINYGKGKMPNFNKKINEYISKNPENIKIIGIDRGEKHLLYYSIIDQNGNNIESMSLNAVDEFGNFVNPEKLEEYEIDNNGKKERRWKYIVNDKEIKVTNYQRKLDELEKERQKSRQSWQNINKIKNLKKGYISFVVKKIVDLAIENNAIIILEDLNFGFKSFRQKIEKNVYQQFEKALIDKLGFVVDKQKQNQRFAPQLSAPFESFQKIGKQTGIVYYVLANNTSKVCPSCQWIKNFYLKYEKKNTIFNLQKNQKLKVFFEQEKNRFRFEYQMSKEYISVYSDVDRQRYDKTKNQNKGGYLEYKNSNQKEIIDKDGVIQKQSITLQLKELFKENHIDLEKEILKQLDNKKEKNSGYTGVYNKFIYLFNLILQIRNAISFREKDYIQCPSCHFDTRKENYLKINDGDGNGAYNIALRGLYLLKGKNGIINNLEKIKLIFSNNDYFQWAKKLKNKK

SEQ ID NO: 15 - protein, 1285 aa, unidentified
  source 1..1285: note = Description of Unknown: Cas12 sequence
MEEKMLKSYDYFTKLYSLQKTLRFELKPIGKTLEHIKNSGIIESDETLEEQYAIVKNIIDKLHRKHIDEALSLVDFTKHLDTLKTFQELYLKRGKTDKEKEELEKLSADLRKLIVSYLKGNVKEKTQHNLNPIKERFEILFGKELFTNEEFFLLAENEKEKKAIQAFKGFTTYFKGFQENRKNMYSEEGNSTSIAYRIINENLPLFIENIARFQKVMSTIEKTTIKKLEQNLKTELKKHNLPGIFTIEYFNNVLTQEGISRYNTIIGGKTTHEGVKIQGLNEIINLYNQQSKDVKLPILKPLHKQILSEEYSTSFKIKAFENDNEVLKAIDTFWNEHIEKSIHPVTGNKFNILSKIENLCDQLQKYKDKDLEKLFIERKNLSTVSHQVYGQWNIIRDALRMHLEMNNKNIKEKDIDKYLDNDAFSWKEIKDSIKIYKEHVEDAKELNENGIIKYFSAMSINEEDDEKEYSISLIKNINEKYNNVKSILQEDRTGKSDLHQDKEKVGIIKEFLDSLKQLQWFLRLLYVTVPLDEKDYEFYNELEVYYEALLPLNSLYNKVRNYMTRKPYSVEKFKLNFNSPTLLDGWDKNKETANLSIILRKNGKYYLGIMNKENNTIFEYYPGTKSNDYYEKMIYKLLPGPNKMLPKVFFSKKGLEYYNPPKEILNIYEKGEFKKDKSGNFKKESLHTLIDFYKEAIAKNEDWEVFNFKFKNTKEYEDISQFYRDVEEQGYLITFEKVDANYVDKLVKEGKLYLFQIYNKDFSENKKSKGNPNLHTIYWKGLYDSENLKNVVYKLNGEAEVFYRKKSIDYPEEIYNHGHHKEELLGKFNYPIIKDRRYTQDKFLFHVPITMNFISKEEKRVNQLACEYLSATKEDVHIIGIDRGERHLLYLSLIDKEGNIKKQLSLNTIKNENYDKEIDYRVKLDEKEKKRDEARKNWDVIENIKELKEGYMSQVIHIIAKMMVEEKAILIMEDLNIGFKRGRFKVEKQVYQKFEKMLIDKLNYLVFKNKNPLEPGGSLNAYQLTSKFDSFKKLGKQSGFIFYVPSAYTSKIDPTTGFYNFIQVDVPNLEKGKEFFSKFEKIIYNTKEDYFEFHCKYGKFVSEPKNKDNDRKTKESLTYYNAIKDTVWVVCSTNHERYKIVRNKAGYYESHPVDVTKNLKDIFSQANINYNEGKDIKPIIIESNNAKLLKSIAEQLKLILAMRYNNGKHGDDEKDYILSPVKNKQGKFFCTLDGNQTLPINADANGAYNIALKGLLLIEKIKKQQGKIKDLYISNLEWFMFMMSR

SEQ ID NO: 16 - protein, 1238 aa, unidentified
  source 1..1238: note = Description of Unknown: Cas12 sequence
MNNYDEFTKLYPIQKTIRFELKPQGRTMEHLETFNFFEEDRDRAEKYKILKEAIDEYHKKFIDEHLTNMSLDWNSLKQISEKYYKSREEKDKKVFLSEQKRMRQEIVSEFKKDDRFKDLFSKKLFSELLKEEIYKKGNHQEIDALKSFDKFSGYFIGLHENRKNMYSDGDEITAISNRIVNENFPKFLDNLQKYQEARKKYPEWIIKAESALVAHNIKMDEVFSLEYFNKVLNQEGIQRYNLALGGYVTKSGEKMMGLNDALNLAHQSEKSSKGRIHMTPLFKQILSEKESFSYIPDVFTEDSQLLPSIGGFFAQIENDKDGNIFDRALELISSYAEYDTERIYIRQADINRVSNVIFGEWGTLGGLMREYKADSINDINLERTCKKVDKWLDSKEFALSDVLEAIKRTGNNDAFNEYISKMRTAREKIDAARKEMKFISEKISGDEESIHIIKTLLDSVQQFLHFFNLFKARQDIPLDGAFYAEFDEVHSKLFAIVPLYNKVRNYLTKNNLNTKKIKLNFKNPTLANGWDQNKVYDYASLIFLRDGNYYLGIINPKRKKNIKFEQGSGNGPFYRKMVYKQIPGPNKNLPRVFLTSTKGKKEYKPSKEIIEGYEADKHIRGDKFDLDFCHKLIDFFKESIEKHKDWSKFNFYFSPTESYGDISEFYLDVEKQGYRMHFENISAETIDEYVEKGDLFLFQIYNKDFVKAATGKKDMHTIYWNAAFSPENLQDVVVKLNGEAELFYRDKSDIKEIVHREGEILVNRTYNGRTPVPDKIHKKLTDYHNGRTKDLGEAKEYLDKVRYFKAHYDITKDRRYLNDKIYFHVPLTLNFKANGKKNLNKMVIEKFLSDEKAHIIGIDRGERNLLYYSIIDRSGKIIDQQSLNVIDGFDYREKLNQREIEMKDARQSWNAIGKIKDLKEGYLSKAVHEITKMAIQYNAIVVMEELNYGFKRGRFKVEKQIYQKFENMLIDKMNYLVFKDAPDESPGGVLNAYQLTNPLESFAKLGKQTGILFYVPAAYTSKIDPTTGFVNLFNTSSKTNAQERKEFLQKFESISYSAKDGGIFAFAFDYRKFGTSKTDHKNVWTAYTNGERMRYIKEKKRNELFDPSKEIKEALTSSGIKYDGGQNILPDILRSNNNGLIYTMYSSFIAAIQMRVYDGKEDYIISPIKNSKGEFFRTDPKRRELPIDADANGAYNIALRGELTMRAIAEKFDPDSEKMAKLELKHKDWFEFMQTRGD

SEQ ID NO: 17 - protein, 1347 aa, unidentified
  source 1..1347: note = Description of Unknown: Cas12 sequence
MASSHFISLDNSFSKFTNLYSLSKTLRFELVPTENTTVMLENNNVFKKDQIIQVKYEKTKPFIDRLHREFIKEALSNYAVSGLQEYFEILRAGGKKANLDSAKKQLRKHVVDQFNATASLWVSRHKDVGFKGEGIELLFKEAVFKLLKEKYGTDMNALIEDNHGKQISIFDSWKGFTGYFDKFQQTRRNLYKDDGKEGRVATRIIDQNLTRFCDNIFVYEKIKDKVSFIDVEKSFGKTCSEVFIPDYYNTCLLQDGIDSYNEFIGGKPLENGEKVQGLNELINLYRQTTGDKVPYFKKLEKQILGEKDEVFIDEITDEDFVPRVLAFYRTVDAKYKLFLKLLDDFVTNQDVYELSQIYISKKGLQEKLYRWLTPSAREVYDEELFEVLKKAKKVNNKDKQKVSGYVPDFVEVLYIKQALENIDAKLIWSDRYYSDGENEGIIDKGFSSWKQFLVILNHEYRQLLSFEDHVIIDKELDFDKEVKQLTDTVEIVSQDKNARTVTYRGGYDVYKAKLAELGQSFEKDTCTKKVIKNFADSVLSMYHFAMMFAVWDDTYPLDVFYTNNEFGYLLYYEDAYKNIVQEYNKLRNYLTKKPYSTEKWKLNFENPTLAAGFDKNKESDNSTVILRQGDKYFLGVMKKGFNKIFDNSQISQTGNSPEAYFEKMVYKYTKDVVTGIPKSSTQVKEVQEHFRNSDEDFFLEECSSVGNFIVPLKITKEIFDLNNKVYAKEDISQAMYRWALNTDEEKNYVKSFQKSYLSLGGSPELYCKSVTLWIGFCLNFLKSYPSAAYFDYSQLRQASDYESVDECYQELNNAGYTILFQNVSEKYVRVKNKNGELYLFQIKNKDWNEGSTGKKNLHTLYFESLFSKENAKQGFPFKLSGNAELFFRPGSIEQTYERRNFPREIPLKRRYSKDGIFFHIPVQVNRTKVGSPNQFNKEVNDFLAGNPNINIIGVDRGEKHLVYYSVISQNGEKIDGGSFNEINGQDYHDKLEKRAKEREQQRRDWETVEGIKDLKKGYISQVVKKLADLAIEHNAIIVMEDLNMRFKQIRGGIEKSVYQQLEKALIDKLSFLVNKGEVDPQKAGHLLKAYQLTAPIDAFKDMGKQTGIMFYTQAAYTSKIDPVTGWRPHLYLKYSSVEKAKDDISRFTKIAYKNDRFEFTYNITDFRTQKEWPLKTEWTVCSCVERFRWNKKLANGKGDYEHYPNVTDDFKKLFDSVGINYLQENIKSQVVNLDENTNVEFFREFIKLFALVCQIRNTNSEEAGNLNDFILSPVEPFFDSRSAEDFGKGLPSNGDENGAYNIARKGMIILNTLSTFKNDHGSCEGLSWGDLYISDTQWDDFAQSFHG

SEQ ID NO: 18 - protein, 1227 aa, unidentified
  source 1..1227: note = Description of Unknown: Cas12 sequence
MDAKEFTGQYPLSKTLRFELRPIGRTWDNLEASGYLAEDRHRAECYPRAKELLDDNHRAFLNRVLPQIDMDWHPIAEAFCKVHKNPGNKELAQDYNLQLSKRRKEISAYLQDADGYKGLFAKPALDEAMKIAKENGNESDIEVLEAFNGFSVYFTGYHESRENIYSDEDMVSVAYRITEDNFPRFVSNALIFDKLNESHPDIISEVSGNLGVDDIGKYFDVSNYNNFLSQAGIDDYNHIIGGHTTEDGLIQAFNVVLNLRHQKDPGFEKIQFKQLYKQILSVRTSKSYIPKQFDNSKEMVDCICDYVSKIEKSETVERALKLVRNISSFDLRGIFVNKKNLRILSNKLIGDWDAIETALMHSSSSENDKKSVYDSAEAFTLDDIFSSVKKFSDASAEDIGNRAEDICRVISETAPFINDLRAVDLDSLNDDGYEAAVSKIRESLEPYMDLFHELEIFSVGDEFPKCAAFYSELEEVSEQLIEIIPLFNKARSFCTRKRYSTDKIKVNLKFPTLADGWDLNKERDNKAAILRKDGKYYLAILDMKKDLSSIRTSDEDESSFEKMEYKLLPSPVKMLPKIFVKSKAAKEKYGLTDRMLECYDKGMHKSGSAFDLGFCHELIDYYKRCIAEYPGWDVFDFKFRETSDYGSMKEFNEDVAGAGYYMSLRKIPCSEVYRLLDEKSIYLFQIYNKDYSENAHGNKNMHTMYWEGLFSPQNLESPVFKLSGGAELFFRKSSIPNDAKTVHPKGSVLVPRNDVNGRRIPDSIYRELTRYFNRGDCRISDEAKSYLDKVKTKKADHDIVKDRRFTVDKMMFHVPIAMNFKAISKPNLNKKVIDGIIDDQDLKIIGIDRGERNLIYVTMVDRKGNILYQDSLNILNGYDYRKALDVREYDNKEARRNWTKVEGIRKMKEGYLSLAVSKLADMIIENNAIIVMEDLNHGFKAGRSKIEKQVYQKFESMLINKLGYMVLKDKSIDQSGGALHGYQLANHVTTLASVGKQCGVIFYIPAAFTSKIDPTTGFADLFALSNVKNVASMREFFSKMKSVIYDKAEGKFAFTFDYLDYNVKSECGRTLWTVYTVGERFTYSRVNREYVRKVPTDIIYDALQKAGISVEGDLRDRIAESDGDTLKSIFYAFKYALDMRVENREEDYIQSPVKNASGEFFCSKNAGKSLPQDSDANGAYNIALKGILQLRMLSEQYDPNAESIRLPLITNKAWLTFMQSGMKTWKN

SEQ ID NO: 19 - protein, 1331 aa, unidentified
  source 1..1331: note = Description of Unknown: Cas12 sequence
MVNKQNERGDFDDLTNLYEISKTLRFELVPVGETDRMLKEENVFKVDENIKRKYQQTKLFFDRIHREFAKEALSVEGILSELEEYLAIFIEWRKDKKIHEKTLNQKEKELRKQVVSAFNAMANKWIERYGDVNLKKKNVEFLFEEGIFRVLKERYGEEDGSTITASDTGEVFSIFDSWKGFTGYFAKFFETRKNFYKDDGTATAIATRIVDENLRRFCDNLIVAQRLTENIDFSEVENNFQIKIKEVLFMEFYNKCLLQDDIDFYNKVIGGETLKTGEKLKGINELVNLHRHKTGEKLPFLKTLDKQILGRKEQFLDEIESEEELLEKLKDFQNVATKKIKVIKSLFGDFVENNENYDLEKIYISKKAFNTISRKWTGETEQFEKLLFESMKSDKPAGLKYDKKENNYKFPDFIAVSYIKDALENFSGEQKFWKDRYYIELELDNQVVWKQFLDIFYWEFSSLFKRSFVNKETGEISEVGCDIFEKKFINLIDDFEYNQKSKILIKDFADSVLSVYQMANYFSLEKKRKWSTEFETDSKFYDDSEIGFRNCFYEDVFEGIVQVYNKLRNYLTKKPFSEEKWKLNFENPTLAAGWDKNKEKDNSTVILRKDEKYFLAIMKKGNNVIFDDRNKALFSQNLEHGKYEKVVYKFAKDVTLGIPKSTTQTKSVIAHFKNSDEDYQITNGSAVGDFLEPLVVTKRIFELNNKIYSKNNLGKVLYRSEVSKDKQKEYIKLFQKKYLVLGGNKNLYRDAVKEWIDFCKSFIKVYPSYKYFDFSLLKEAVEYNSVDEFYKELNSYGYAISFQDISCDYIEEKNKNGELYLFQIKNKDWNKGSTGMKNLHTLYFESLFSEENIKNNFVTKLNGGAEIFYRPKTSKEKLGRKKIVRNGQEVFVVNHKRYSEDKIFFHCSIALNRGKGKLLKFNARINDLLANNPDINVIGVDRGEKHLAYYSIIDQKCKILDSGTLNEVGAKVDYHEKLSNRAKKREDGRRDWGWGQIEDIKNLKKGYVSQVVHKLAELIIKYNAILVFEDLNMRFKQIRGGIEKSIYQQLEKALIDKLNFLVKKGEKDSKSAGHLLKAYQLAAPFETFDKMGKQTGVIFYTQASYTSKIDPITGWRPNLYLKHSNANDSQKKIAKFSRIEFINDRFEFEYDLKKFIEMKEVPENTKWTLCSCVQRYRWNRKLNANKGGYDSYNDLTKNFKALFESVGIDIKKNIKEQIVKMEIKGNEKFFKSFIFYWQLLCQIRNTDELKKGDDNDFILSPVEPFFDSRKKNGDDLPKNGDDNGAYNIARKGVIVLNKISEFSKQNGNCEKCGWKELYVSAKDWDDFVQAK

SEQ ID NO: 20 - protein, 1275 aa, unidentified
  source 1..1275: note = Description of Unknown: Cas12 sequence
MQNKQSFADFTNLYSLSKTLRFELKPIGQTQAMLDENKIFEVDENRKKAYDKTKPYFDRLHREFINESLSNAQLKGISEYFETFKQFRSNQNNKDLKELINKQQKFLRHQIVTLFDENGKHWATTKYAHLKIKKKNLDILFDEQVFYILKERYGSEKETQLVDKETGAVTSIFDNWKGFTGYFTKFFETRKNFYKSDGTSTALATRIIDQNLNRFFDNLETFHKIKDKIDVKEVEIFFKLKADNVFSIDFYNQCLLQNGIDKYNDFLGGQTLENGEKQKGINEIINKYRQDNKDQKLPFLKKLDKQILSEKDRFINEIESKEEFFQVLTEFYQSATVKVTIIKTLLNDFVHNTDKYKLEKIYLTKEAFNTIANKWTDETQIFEDNLDLVLKNKKITAKQDFIPLAYIKEALEVIEKDRKFFKDRYYNDPQIGFFPDQSYWEQFLAILNFEFMTHFQRVAKDKITGKKIELGYFVFEKRIKELLDSDPSLNSQSKIIIKEFADEVLHIFQMAKYFALEKKREWKGDYYQLDDQFYNHIDYGFKDQFYENAYEKIVQPYNKIRNYLTKKPYSDVKWKLNFGNPTLANGWDKNKEADNTAVILKKDGNYYLGVMKKGKNKIFSDQNKEKYKAYNSAYYEKLVYKLFPDPSKMFPKVCFSKKGLNFFQPSEEILRIYKNNEFKKGNTFSISSMQKLIAFYIDCLGLYEGWKHYEFKNIKDVRQYKENIGEFYADVAESGYKLWFEKISEEYITQKNQLGELFLFQIYNKDFAKKTTGRKNLHTIYFEELFSQTNIDNNFPFKLNGQAELFYRPKSLEKIEEKRNFKRSIVNKKRYTQNKIFFHVPITLNRTSENIGRFNVRVNNFLANNSNVNIVGVDRGEKNLAYYSIIKQNGEVLKSGSLNIINGVDYHALLTDRAQRREQERRNWQDVESIKDLKRGYISQVVHELVSLAIKYNAIIVMEDLNMRFKQIRGGIEKSTYQQLEKALIEKLNFLVNKEETDSNQAGNLLNAYQLTAPFKTFKDMGKQTGIIFYTQASYTSKIDPLTGWRPNIYLRYSNAKQAKADILMFTNIYFSEKKDRFEFTYDLEKIDDKRKDLPIKTEWTVCSNVERFSWEKSLNNNKGGYVHYPIQDSNGEESITSKLKKLFMDFGIDLTDIKTQIESLDTNKKDNANFFRKFIFYFQLICQIRNTQVNKSDDGNDFIFSPVEPFFDSRFADKFRKNLPKNGDENGAYNIARKGLIILHKISDYFVKEGSTDKISWKDLSISQTEWDNFTTDK

SEQ ID NO: 21 - protein, 1313 aa, unidentified
  source 1..1313: note = Description of Unknown: Cas12 sequence
MDKQKNKLQNFTNLYELSKTLRFELKPVGETQHLLEENKVFGIDGNIKKKYEATKPFFDRLHRKFVKEALVNIALGGLDNYLEVYKKFTNDRKDKENQKELEKQEKLLRKQIKIFFDSQANQWKEKYNKINFKKSGLNILFEESIFQLLKEIYGKEDDAFLKNDDNEFIFDKDGNKISIFDSWKGFTGYFKKFFETRKNFYKDDGTSTAIATRIIDQNLRRFCDNIFIYNKIKNKLDFSSLEKEQDVVLEEIFTTAYYMDCILQDDIDLYNGVLGGETLDDGTKIKGLNEIINKYRQDNKGDKIPFFKKLDKQILSEKDRKFLDEIESEEELAELLKIFINNTEAKVKVFDELVNQLCVNDSDFELDKIYISKEAFNTISHKWTNQTHEFERVLFEEMKPDKITGLDYKKAEDKYKFPDFIALKYIIKSLNTLDKDSEFWKSHYYKTEENQNAILSLEEKVGEQFLQIYKYELQRLHSRNVNVENKDGKMKEKEIGLDYSLTTVKELLKNFKLTDKSKIIIKDFADNVLQYYQLAKYFSVEKNREWNYTKLELADFYINPDFGYEIFYGNAYEEIIQIYNKLRNYLTKKPFSEEKWKLNFENPTLAGGWDKNKERGNATVILRKNEKYYLGIMAKGYNDIFTDKNKDKFDGEGYEKMVYKLFPGPNKMMPKVCFSKKGLDFFEPSEKIIDIYKDGKFKQGDTFSIDSMQQLIDFYKRALREYNGWKMYDFSKLKDTNDYTTNIGEFYNDVACAGYKVWFDNISEEYIQEKNENGELYLFEIHNKDWNLKDEKKKTGTKNLHTLYFESLFSDENALRDFVMKLSGEAELFFRPKTNADKLGYRKDKKGNKVVKNKRYSEDKMFLHLSINLNRGKGQAFWFNRNINNFLANNSDINVIGIDRGEKHLAYYSVISQQGEILDNGSLNEIAGVDYYAKLSKRAKEREGQRKDWQAVSDIKNLKKGYISQVVRKLADLAIEHNAIIVLEDLNMRFKQIRGGIEKSIYQQLEKALIEKLNFLVNKKEIDSDKAGNLLRAYQLTAPFETFQKMGKQTGIIFYTQASYTSKIDPLTGWRPNLYLKKGNAKINKEQIEKFSKIEFTNNRFEITYDLKNFGDKKKKYPQKTKWTLCSSVERWRWDRKLNNNKGGYIHYEDLTTEFKSLFEKFEIDIEGDILEQIKTIDENDRNNARLFSGFIYLWGLLSQIRNTDGELDEKIKKLEREDKNEEISEKEKFDVDFILSPVEPFFDSRTPEKFGENLPKNGDDNGAYNIARKGIITLERIKKFYELSDKEREKLKYPDLFITNAEWDDFATKRDS

SEQ ID NO: 22 - protein, 1155 aa, unidentified
  source 1..1155: note = Description of Unknown: Cas13 sequence
MDNNTTLEKTELGLGITYNHDKVEDKHYFGGFFNLAQNNIDLVAQEFKKRLLVQGKDSINIFSNYFSDQCSITNLERGIKVLSEYFPVIFYFDLDENNKSKSIRQHIILLLDTINNLRNYYTHYYHKKVIIDDALYPLLDTILLKVVLEIKKKKLKEDKTKQLLKKGLEKEMAILFNLMKKEQKEKKIKGWNIDKNIKGAVLNRAFSHLLYNDGISDYRKSKSNTEDENLKDTLSESGILFLLSFFLNKKEQEQLKANIKGYKGKIASIPDEEITLKNNSLRNMATHWTYSHLTYKGLKHRIKTDHEKETLLVNMVDYLSKVPNEIYQNLSEQNKSLFLEDINEYMRDNEENNDSSEASRVIHPVIRKRYENKFAYFAIRFLDEFAEFPTLRFMVNVGNYIHDNRKKDIGGTSLITNRTIKQQINVFGNLTEIHKKKNDYFEKEENKEKILEWELFPNPSYHFQKENIPIFIDLEKSKETNELAKEYAKEKKKIFGSSRKKQQNTAKKNREAIINLVFDKYKTSDRKTVTFEQPTALLSFNELNAFLYAFLVENKTGKELEKIIIEKIANQYQILKNCSSTVDKTNDSIPKSIKKIAHPTTDSFYSEGKKIDIEKLERDIKIEIEKTNEKLETIKENETSAKNYKRNERDIQKRKLYRKYVFFTNEIGIEATWITNDILRFLDNKENWKGYQHSELQKFISQYDNYKKEALGLLESEWNLESEAFFGQKLKRIFQSNFTFETFYKKYLDNRKDTLETYLSAIENLKTMTDVPPKILKKSWAELFRFFDKKIYLLSTIETKINELITKPINLSRGVFDEKPTFINGKSPNKENDQHLFANWFIHAKEQTIFQDFYNLALETPKEINNLKKQNYKLERSINNLKIEDIYIKQMVDFLYQKLFEQSFKGSLQDLYTSKEKREVEKSKAKNEQTPDESFIWKKQVEINALNGRIIAKTKIKDIGKFKNLLTDNKITHLISYDNRIWNFSLDNDGDTTKKLYSLNTELESYERIRREKLLKQIQEFEQFLLKQETEYSAERKHPEKFEKDGNPNFKKYIIEGMLNKITPVNEIEELEILKSKEDVFKIDFNEIVKLNNESIKKGYLLIMIRNKFAHNQLIDKNLFTFSLQLYSKNENENFSEYLDKVCQKIIQEFIEKLK

SEQ ID NO: 23 - protein, 1134 aa, unidentified
  source 1..1134: note = Description of Unknown: Cas13 sequence
MNETDYLAKRLEYNYASIEDKHYFGGYFNLAQNNINDLSKAFKEKFGMKPKSCILDFFTQDKAIAEYQLGVEFLQKNLPVIRYLYLPTSHKRFENVPKNQLISEQRNYFKNSLKVLKNLIRDYRNFYTHHFHKPIPVFPETYKLLDDLFLAVANDVKKHRMKTDASKQLLKKGLIEELAQLEKLKLEDLKKLKREGKKVNLNDKEAITNAILNDSFSHLLPKENTISKYYSAVPTEDIDTENGVTISESGIIFLLGLFLTKKQSEDLRSRVKGFKAKLIVNPENPINKKNNSLKYMATHWVFGYLGFKGLKNRFTTTFTKDTLLAQIVDELSKVPDELYQVLPEELKNEFLEDMNEYLKEENSESLDKATVIHPVIRKRYENKFAYFALRFLDEFVDFPTLRFQLHLGNYVHDKREKPIEGTKYVTERIVKEKIKAFAKLSEAAQLKQKYFEEKENHQSIGLQLYPNPSYNFVGNNIPIHLNLNEHFFPKEVKIVAGRLKKRNSSYKSDHPEEYKVRTDNKIKPDAILQDLGKPEKLAPVAMLSLNELPALLHLVLTKKTPEEIEIIIAQKIAERYNVLTNYKAGDDISKGQITKNLLKAKQKKEVNLDKLQLAIEKEIAVTNDKLQTIALHIKERNDPKQKRKYVFTNKEIGLQVTWLANDLKRFMPKGSRQNWRGQHHSQLQKSLAFYDIQPKEPLSLLEEVWDFKNEAYLWNNGIRRSFDKRDFISFYTSYLNNRKETFQRFKDQLNGIRSNKKILDKFIKQQHLWNLFHKRLYVIDTIEEQVEKLLVKPMQFPKGVFDHKPTYIKGKSIQENPECFADWYVAWNQHTDYQKFYSWDRDYKSAYLSGEQEKTEKRFIRVQGSKINKVKQQDVLLAKMASIIFNELYLPEDAEHLDLNLSDIYKTQTERKAEIEAALIQSHKTTGDNSANIIKSTSAWTLTVPYCSKNIYEPQVKLKELGKFKKFIASQKVQTLFEYKPQKIWNKTELEEVLELKANSYEVIRRDYLLKSIQEFEKYMIKKLPTLIDTNEHPNFNKYLTTFLKSLELVSEEDAKWLISKKDFDTTPIDELKKQSKIMEKAFLLVMIRNKFSHNQLPRKIYYDEIYKNVPNAVSINFNELFLEYTNQTILEFK

SEQ ID NO: 24 - protein, 1145 aa, unidentified
  source 1..1145: note = Description of Unknown: Cas13 sequence
MESIIGLGLSFNPYKTADKHYFGSFLNLVENNLNAVFAEFKERISYKAKDENISSLIEKHFIDNMSIVDYEKKISILNGYLPIIDFLDDELENNLNTRVKNFKKNFIILAEAIEKLRDYYTHFYHDPITFEDNKEPLLELLDEVLLKTILDVKKKYLKTDKTKEILKDSLREEMDLLVIRKTDELREKKKTNPKIQHTDSSQIKNSIFNDAFQGLLYEDKGNNKKTQVSHRAKTRLNPKDIHKQEERDFEIPLSTSGLVFLMSLFLSKKEIEDFKSNIKGFKGKVVKDENHNSLKYMATHRVYSILAFKGLKYRIKTDTFSKETLMMQMIDELSKVPDCVYQNLSETKQKDFIEDWNEYFKDNEENTENLENSRVVHPVIRKRYEDKFNYFAIRFLDEFANFKTLKFQVFMGYYIHDQRTKTIGTTNITTERTVKEKINVFGKLSKMDNLKKHFFSQLSDDENTDWEFFPNPSYNFLTQADNSPANNIPIYLELKNQQIIKEKDAIKAEVNQTQNRNPNKPSKRDLLNKILKTYEDFHQGDPTAILSLNEIPALLHLFLVKPNNKTGQQIENIIRIKIEKQFKAINHPSKNNKGIPKSLFADTNVRVNAIKLKKDLEAELDMLNKKHIAFKENQKASSNYDKLLKEHQFTPKNKRPELRKYVFYKSEKGEEATWLANDIKRFMPKDFKTKWKGCQHSELQRKLAFYDRHTKQDIKELLSGCEFDHSLLDINAYFQKDNFEDFFSKYLENRIETLEGVLKKLHDFKNEPTPLKGVFKNCFKFLKRQNYVTESPEIIKKRILAKPTFLPRGVFDERPTMKKGKNPLKDKNEFAEWFVEYLENKDYQKFYNAEEYRMRDADFKKNAVIKKQKLKDFYTLQMVNYLLKEVFGKDEMNLQLSELFQTRQERLKLQGIAKKQMNKETGDSSENTRNQTYIWNKDVPVSFFNGKVTIDKVKLKNIGKYKRYERDERVKTFIGYEVDEKWMMYLPHNWKDRYSVKPINVIDLQIQEYEEIRSHELLKEIQNLEQYIYDHTTDKNILLQDGNPNFKMYVLNGLLIGIKQVNIPDFIVLKQNTNFDKIDFTGIASCSELEKKTIILIAIRNKFAHNQLPNKMIYDLANEFLKIEKNETYANYYLKVLKKMISDLA

SEQ ID NO: 25 - protein, 1147 aa, unidentified
  source 1..1147: note = Description of Unknown: Cas13 sequence
MEDKTTGAGISYDHTLMEDKHFFGGFLNLAQNNIDALLKAFKERFNVRYQSKQFAEVCFSDKLPDQDYLDRTLFLETHLPFIKYIGGKEANNRGTFRKNITLFFESIEQLRNFYTHYYHKPILFPEELYENLDRIFVEVSKEVKTHKVKNDQTRHLLTKNLANELDIRYKKNVEKLKELKAQGKKVNIHDKEAIKNSVLNNAFNHLIYKKEEDVFATEAYKSKYNLEDPSKNGISLSQSGLLFLLSMFLNKKDIEALKSRVKGFKAKIIRDGEENISGLKFMATHWVFSSLSFKNVKHKLSTDFHKETLLIQIVDELSKVPDEVYKTFDKQTQEEFIEDINEYMKVGNKDLSLEESTVIHPVIRKRYDNKFNYFALRFLDEFAGFPTLRFQVHIGNYIHDRRIKNIDGTAFQTERSVKERIKVFGKLSQMSNLKAEYVSGLMDEPVDTGWEIFPNPSYNIIENNIPIYIEMGDHFNDEVLQSKMARKKQKPEELKDRNSAKASKESMIQTLQNDKGLMDVITVSPTAQLSLNELPAILYELLVKKTPAKTIEKKLVGKLNQRLKEIKNYNPEKPLPASQISKRLRLNREEGSINTKKIIALLQKELNYTQEKLDLLEKNRKEYGKKVDGKILRKYVFGLKEIGNLATDMAMDIKRFMPANVRKEWKGYQHSQLQQSLAFYDKRPEEAFNILQEVWDINREKSLWDTWILNAFQTSGNFERFFELYHEGRKKYIQQQLENIDRYTDNKKFLQKFINQQFPTNFLEKRLYTLESLEIEKLKILSKPFILPRGTFDEKPTFIMGEKVTENPELFADWYTYGYQQHEFQKFYSWPRDYKDLLQNEQKRDPDFAENKKGLSDLKQLELLQLKQDIIIKKIKTQDLYLKLIMDALFIEVFGQEADISLNDLYLTQEERLEKEKLALKQHQRVEGDDSPNVIKDNFIWSKTMPYKHDKIYEPQVRLKDFGKFKHFLLDDKVAKILSYDLQETWNKNELEIQINTGQDSYEVIRREELLKEIQLLEKQILETFSHTLDEHPKEFEDEKGNPNFKMYMANGVIRKGSSTTAKDEADWLEHEKDFDNLSLEIFNSKSEITQLTFLIVLIRNKFGHNQLPIKQFYEIIQNEYSITGETISRLYLNFIIYAKARLKDLM

SEQ ID NO: 26 - protein, 1133 aa, unidentified
  source 1..1133: note = Description of Unknown: Cas13 sequence
MEEKLGKGVEYNPFKKEDKYYFGGYFNLAENNINEVFKEVKKRLGETNSSSNIELLNNVFRKEMSLVDYEKWVNAFADYFPIVNYLDRETIKKGEKVVEVPREKRIECFRDMFKGLINTISQLRHYYTHYHHEPIEIDDKILSFLDEVLFNTIITTKNKYLKTDKTKELIKDSLQEELDILCKLKVKYLESKRKRFDRKDKGAIENAVYNDVFRRFIYKDEKGNESLKDIIRTKQIKVHQNSSYLELPISSSGIIFLLSLFLNKKEVESLKSNIRGYKGKSKSEETTPEKNGLLFMTTHRIYSVLAYKGLKKRIKTSVKGDKETLLMQMIDEVSKVPHCIYQNLDQTLQATFIEDWNEYFKDNEENEENLENSRVLHPVIRKRYEDKFNYFAIRFLDEYAEFPSLRFQVNLGNYVHHKATKKFGNSEVTTERVIKDKITVFGRLSEVNKAKADFFKNETELDPAWELFPNPSYEFPKEKGNNDKDAGKIGIQVKLLNKDIEAVLNESKNTLNNKTRKSDKISKKEIINKIVQINDDTKYNNKNIIYQGNAIAYLSLNDIHSLLYELLVIGTKGDKLERKVVEKIQQQVTEIRNKDTSAKILSKYKDSEESNTIDKKKLVIDLKYEYDKLQDLLKEHKNREEDYIQTKKKKKDSPKRKYILYHNEKGQVAVWLSNDIKRFMPQNFKEKWKGYQHSEFQKSLAYYETNKEMLKIILQDLDLEQFPFDIKSCFYKNTLEDFYNRYLSLRISYLENVIDRVECFSNEPKAFKSVLKECFVFLKKQNYTNHSLDEQVKKILANPIFIERGFLDTKPTMIQGVKFSENKGCFADWFVHYKEYEHYQKFYDTNLYPVESIEDKERQKLEATIKKQQKNDVFTLLMIKKIFNDLFNQDFEANLYEMYQSKEEREKNQLVAKETQNRNLNFIWNKPIAIDLFDGKVKIDEVKLKDVGSFRKYENDKRVQTFITYIPEIQWIPYLPNTWEGINLPVNVIERQIDRYEKVRSEELLKEVQAIEKYIYEQVNDKTELLQNGNQNFKNYLVNGLLKQIQGIDVSNFKFINQQKFETINVKDLDNEASALEQKVYVLINIRNQFSHNQFPKSAFYQFCQKILSIEEDELFADYYLRLFKLLRNELLD

SEQ ID NO: 27 - protein, 1156 aa, unidentified
  source 1..1156: note = Description of Unknown: Cas13 sequence
MNTRVTGMGVSYDHTKKEDKHFFGGFLNLAQDNITAVIKAFCIKFDKNPMSSVQFAESCFTDKDSDTDFQNKVRYVRTHLPVIGYLNYGGDRNTFRQKLSTLLKAVDSLRNFYTHYYHSPLALSTELFELLDTVFASVAVEVKQHKMKDDKTRQLLSKSLAEELDIRYKQQLERLKELKEQGKNIDLRDEAGIRNGVLNAAFNHLIYKEGEIAKPTLSYSSFYYGADSAENGITISQSGLLFLLSMFLGKKEIEDLKSRIRGFKAKIVRDGEENISGLKFMATHWIFSYLSFKGMKQRLSTDFHEETLLIQIIDELSKVPDEVYHDFDTATREKFVEDINEYIREGNEDFSLGDSTIIHPVIRKRYENKFNYFAVRFLDEFIKFPSLRFQVHLGNFVHDRRIKDIHGTGFQTERVVKDRIKVFGKLSEISSLKTEYIEKELDLDSDTGWEIFPNPSYVFIDNNIPIYISTNKTFKNGSSEFIKLRRKEKPEEMKMRGEDKKEKRDIASMIGNAGSLNSKTPLAMLSLNEMPALLYEILVKKTTPEEIELIIKEKLDSHFENIKNYDPEKPLPASQISKRLRNNTTDKGKKVINPEKLIHLINKEIDATEAKFALLAKNRKELKEKFRGKPLRQTIFSNMELGREATWLADDIKRFMPDILRKNWKGYQHNQLQQSLAFFNSRPKEAFTILQDGWDFADGSSFWNGWIINSFVKNRSFEYFYEAYFEGRKEYFSSLAENIKQHTSNHRNLRRFIDQQMPKGLFENRHYLLENLETEKNKILSKPLVFPRGLFDTKPTFIKGIKVDEQPELFAEWYQYGYSTEHVFQNFYGWERDYNDLLESELEKDNDFSKNSIHYSRTSQLELIKLKQDLKIKKIKIQDLFLKLIAGHIFENIFKYPASFSLDELYLTQEERLNKEQEALIQSQRKEGDHSDNIIKDNFIGSKTVTYESKQISEPNVKLKDIGKFNRFLLDDKVKTLLSYNEDKVWNKNDLDLELSIGENSYEVIRREKLFKKIQNFELQTLTDWPWNGTDHPEEFGTTDNKGVNHPNFKMYVVNGILRKHTDWFKEGEDNWLENLNETHFKNLSFQELETKSKSIQTAFLIIMIRNQFAHNQLPAVQFFEFIQKKYPEIQGSTTSELYLNFINLAVVELLELLEK

SEQ ID NO: 28 - protein, 1036 aa, unidentified
  source 1..1036: note = Description of Unknown: Cas13 sequence
METQILGNGISYDHTKTEDKHFFGGFLNTAQNNIDLLIKAYISKFESSPRKLNSVQFPDVCFKKNDSDADFQHKLQFIRKHLPVIQYLKYGGNREVLKEKFRLLLQAVDSLRNFYTHFYHKPIQLPNELLTLLDTIFGEIGNEVRQNKMKDDKTRHLLKKNLSEELDFRYQEQLERLRKLKSEGKKVDLRDTEAIRNGVLNAAFNHLIFKDAEDFKPTVSYSSYYYDSDTAENGISISQSGLLFLLSMFLGRREMEDLKSRVRGFKARIIKHEEQHVSGLKFMATHWVFSEFCFKGIKTRLNADYHEETLLIQLIDELSKVPDELYRSFDVATRERFIEDINEYIRDGKEDKSLIESKIVHPVIRKRYESKFNYFAIRFLDEFVNFPTLRFQVHAGNYVHDRRIKSIEGTGFKTERLVKDRIKVFGKLSTISSLKAEYLAKAVNITDDTGWELLPHPSYVFIDNNIPIHLTVDPSFKNGVKEYQEKRKLQKPEEMKNRQGGDKMHKPAISSKIGKSKDINPESPVALLSMNEIPALLYEILVKKASPEEVEAKIRQKLTAVFERIRDYDPKVPLPASQVSKRLRNNTDTLSYNKEKLVELANKEVEQTERKLALITKNRRECREKVKGKFKRQKVFKNAELGTEATWLANDIKRFMPEEQKKNWKGYQHSQLQQSLAFFESRPGEARSLLQAGWDFSDGSSFWNGWVMNSFARDNTFDGFYESYLNGRMKYFLRLADNIAQQSSTNKLISNFIKQQMPKGLFDRRLYMLEDLATEKNKILSKPLIFPRGIFDDKPTFKKGVQVSEEPEAFADWYSYGYDVKHKFQEFYAWDRDYEELLREELEKDTAFTKNSIHYSRESQIELLAKKQDLKVKKVRIQDLYLKLMAEFLFENVFGHELALPLDQFYLTQEERLKQEQEAIVQSQRPKGDDSPNIVKENFIWSKTIPFKSGRVFEPNVKLKDIGKFRNLLTDEKVDILLSYNNTEIGKQVIENELIIGAGSYEFIRREQLFKEIQQMKRLSLRSVRGMGVPIRLNLK

SEQ ID NO: 29 - protein, 1161 aa, unidentified
  source 1..1161: note = Description of Unknown: Cas13 sequence
MENQTQKGKGIYYYYTKNEDKHYFGSFLNLANNNIEQIIEEFRIRLSLKDEKNIKEIINNYFTDKKSYTDWERGINILKEYLPVIDYLDLAITDKEFEKIDLKQKETAKRKYFRTNFSLLIDTIIDLRNFYTHYFHKPISINPDVAKFLDKNLLNVCLDIKKQKMKTDKTKQALKDGLDKELKKLIELKKAELKEKKIKTWNITENVEGAVYNDAFNHMVYKNNAGVTILKDYHKSILPDDKIDSELKLNFSISGLVFLLSMFLSKKEIEQFKSNLEGFKGKVIGENGEYEISKFNNSLKYMATHWIFSYLTFKGLKQRVKNTFDKETLLMQMIDELNKVPHEVYQTLSKEQQNEFLEDINEYVQDNEENKKSMENSIVVHPVIRKRYDDKFNYFAIRFLDEFANFPTLKFFVTAGNFVHDKREKQIQGSMLTSDRMIKEKINVFGKLTEIAKYKSDYFSNENTLETSEWELFPNPSYLLIQNNIPVHIDLIHNTEEAKQCQIAIDRIKCTTNPAKKRNTRKSKEEIIKIIYQKNKNIKYGDPTALLSSNELPALIYELLVNKKSGKELENIIVEKIVNQYKTIAGFEKGQNLSNSLITKKLKKSEPNEDKINAEKIILAINRELEITENKLNIIKNNRAEFRTGAKRKHIFYSKELGQEATWIAYDLKRFMPEASRKEWKGFHHSELQKFLAFYDRNKNDAKALLNMFWNFDNDQLIGNDLNSAFREFHFDKFYEKYLIKRDEILEGFKSFISNFKDEPKLLKKGIKDIYRVFDKRYYIIKSTNAQKEQLLSKPICLPRGIFDNKPTYIEGVKVESNSALFADWYQYTYSDKHEFQSFYDMPRDYKEQFEKFELNNIKSIQNKKNLNKSDKFIYFRYKQDLKIKQIKSQDLFIKLMVDELFNVVFKNNIELNLKKLYQTSDERFKNQLIADVQKNREKGDTSDNKMNENFIWNMTIPLSLCNGQIEEPKVKLKDIGKFRKLETDDKVIQLLEYDKSKVWKKLEIEDELENMPNSYERIRREKLLKGIQEFEHFLLEKEKFDGINHPKHFEQDLNPNFKTYVINGVLRKNSKLNYTEIDKLLDLEHISIKDIETSAKEIHLAYFLIHVRNKFGHNQLPKLEAFELMKKYYKKNNEETYAEYFHKVSSQIVNEFKNSLEKHS

SEQ ID NO: 30 - protein, 848 aa, unidentified
  source 1..848: note = Description of Unknown: Cas13 sequence
  VARIANT 821..822: note = MOD_RES - Any amino acid
MEKTQTGLGIYYDHTKLQDKYFFGGFFNLAQNNIDNVIKTFILKFFPERKDKDVNAAQFLDICFKDNDADSDFLKKTKFLRMHFPVIGFLASNNDKAGFKRKFSLLLKAISELRNFYTHYYHQPIEFPSELFELLDDIFVETTSEIKKLKKKDDKTQQLLNKNLSEEYDIRYQQQIERLKELNAQGKKIPLNDETAIRNGVFNAAFNHLIYKDGGDLKPSRVYQSSYSEPDPAENGTSLSQSSILFLLSMFLERKETEDLKSRVKGFKAKFIKNGEEKISNLKLTATHWVFSYLCFKGIKQKLSTEFHEETLLIQIIDELSKVPDEVYSAFGAKTKQKFVEDINEYMKEGNADLSLEDSKVIHPVIRKRYENKFNYFAIRFLDEYLSSTSLKFQVHVGNYVHDRRIKNINGTDFQTERVVKDSIKVFGRLSKISNLKADYIKEQLSLPNDSNGWEIFPNPSYVFIDNNVPIHIQTDEATKNGIKLFKDTRRKEQPEELQKRKGKLSKHNIVEIIFKETKGKDKPRVDEPLALLSLNEIPALLYQILEKGATPEDIELIIKNKLAERFEKIKNYDPETPAPASQISKRLRNNTTAKGQETLNAEKLSILIEREIEDTETKLDAIEEKRRKAKKEYRRNSPQKSIFSNSELGRIAAWLADDIKRFMPAELRKNWKGYQHSQLQQSLAYFEKRPQEAFLLLKEGWDTSDGSSYWNIWVINSFSETEDFEKFYENYLRKRAKYFSELAGNIKQHTHNAKFLRKFIKQQMPADLFPKRHYILKDLETEKNKVLSKPLVFSRGLFDSNPTFIKGVKVTENPELFAEXXNGIATGTKRNIPSSISMAGKETIMSF

SEQ ID NO: 31 - protein, 1241 aa, unidentified
  source 1..1241: note = Description of Unknown: Cas13 sequence
  VARIANT 644..727: note = MOD_RES - Any amino acid
MEQNKLGKGIDYNPFKTVDKHYFGGFFNLADNNIQEVFDEINIRYKNGNLKPKVAIERYTTENTSLVEYEKFVAILTEYFPIVKEIDQKNKKDSNDKVIEKTRIERITDFRDAFILFIETIEKLRSYYTHYQHDDITIDNQLFIHLDKILLNTVLETKKKYLKTDKTKELLKNSLQAELKELYHLKINQLEQKKNEVDALIKEQKSKGKKTDKPFKYSKDRDQIINSIYNDAIRPFLYENANKVELSDKKKTAFNEKDASASERDFNLPISSSGIIFLLSCFLNRKEIEDLKANIKGYKGKVIKGETFDLEKNSIRFMATHRIYSVMCYKGLKNKIRTSESATKETLLMQMIDELSKIPDIVYKNISTDLQNTFTEDWNEYYKDNIENNENLENSKVIHPVIRKRYEDKFNYFAIRFLDEFVDFPSLRFQVHLGNYIKHSMPKNIGSVTTTREIKNKIFVFGKLNEINQSKNDFFNKNKEEEQETNWEIFPNPNYHFPMENSDELKNANKIGIYIDLKDKRKKDTLNEAIKKREKETSIYKKDLVHQIIDKNLDMHIGQPVAYLSMNDIHAIIFSILSQNVFTKDNKLNGGDIEKKIKDQINNQITEITEKDASIKILKNHSDNNSNYPNTHKLYDDISNEIEXXXXXXXXXXXXXXXXXXXXXXXXXXXXXXXXXXXXXXXXXXXXXXXXXXXXXXXXXXXXXXXXXXXXXXXXXXXXXXXXXXXXNEIEVLDKLMQKHEKRVKEYINTQEDKKYKPARKHILYNSEKGEIATWLANDIKRFFPKEFKENWKGHYHSEFQRNLAYYETNKKEVKTILNDLDYRKEIPFIDFSKNTLADFYFEYLKKRKIYHKNLWVEVNKLIKGENINKEKLFDNCFRIYKRKNYVSNVIDEKVNTILSNPIFIERGFIDEKPTIIPKMPLEGNEEHFAAWFVAFKSFKNNEFQNFYDTNKYPLETKDKTNSELKKIQTKTYNQKKNDWATWLIVQYIFKDIFSTDLQNVKLSELFQTREQRIQNQVKALDGERNQNFIWNRTIDLQLNEKIKIPNVKLKDIGNFRKYVNDSRVEAFLRYNDITQWMAYLPSNWQKEDESKPKPVNVIQLQLDDYEKIRREELLKEVQKLEKTIYNNTNVKTVLLQDGNPNFKNYVLNGLLEEIKGINISAFTVLHEKTNFDKIDFNVLENCSEIEQSATLIILIRNKFAHNQLPSSDCYQFCSKILTRDTEQTYANYYLKLFMILKDKL

SEQ ID NO: 32 - protein, 1147 aa, unidentified
  source 1..1147: note = Description of Unknown: Cas13 sequence
MEETTTMGKGVAYDHTLFKDKHYFAGYLNLAVNNIENVFKTVYKNRFDIKQHNLYKILDSLDGQISEPDYIERVSFLKQYFPVLHYLDLHPDNKRFTKEEDKVKARRRYLINNLRLLIETLSKLRDFYTHYYHKPLSIEQNTFSLIDNIFLNVVIDVKRQKKKNDHTRQLLKDSLKEEMDILYQKTKASLKEKQKENTRIKLDSETINNTIFNNSFSHLIYRRKKADNDILSASCKSEYKGEPTENGINVSVDGLLFFLGIFLSRKESNDLRGRIKGFKGTVIKDLPDFPNEKNNSLKFMATHWVFTYLNIKPIKQKLNTNFSRETLLLQIVDELTKIPNEIYRNLCFKKQQEFVEDINEYIKEGDDIDTLNSSTVIHPVIRKRYENKFNYFVLRYLDEFVSFNSLRFQIYLGNYVHHIQRKKLSGTEYETERVIKEKINVFGKLSEVSNIKGDYFIQNNPDNEALGWEIYPNPSYNFTGNNIPIYFDINDQDKEKINEYKSIRNFSEKRILRKKNKKNKQEIFDLINNTLTTRVFTAEPTAILSLNELPALLYTILCENKTASEIENLLRRTYLKRLNTIKNYQPGTLPQSKITKNLNKSTNQESLDVSKLIKAMKHEISISNEKLTLIKKNQNEVKDTSHRRKYVFNSKELGIEATWLANDLKRFMPKKVRENWKGYMHSQLQNSIAYYSQKPKEALSILSSVWNFNDDNYIWNEGIKKAFNEKEFEKFYCKYLASRNKTLEKLKENLDNLEYKTDKRKLDKFIKQQNLDCLFHIRTYTIDSTQEQINKLLAKPLVFPRGIFDSKPTFVKNESVTEKPELFADWYTYTYKEHPLQEFYSFTKDYECNFKKEKLTVKEFVKNQEQLNPEEQLNLFKLKEDLSIKCIKNQDLFLKLVVDNIYNKIFEYNIDISLKNLYISRKERIAIGLKAKELNQINDSYIWGKTILYQDKQIRETKVQLKDINKIKRFLEEDKVKQILSYDINKQWEIEELKYELYIKPNSYEVIRREKLFKAIQEFESYILTINNFDGSNHPSILEYNSNPRFKHYVVNGLLLKKGLATNEEIEWLLAKGQKEFNTFDKSIVEKPEIIQKAFLLVLIRNKFAHSQLPIKEYYEMIRSYTKNIENLNTTEIIFQFTTNTINELKR

SEQ ID NO: 33 - protein, 1108 aa, unidentified
  source 1..1108: note = Description of Unknown: cas12b sequence
MATRSFILKIEPNEEVKKGLWKTHEVLNHGIAYYMNILKLIRQEAIYEHHEQDPKNPKKVSKAEIQAELWDFVLKMQKCNSFTHEVDKDVVFNILRELYEELVPSSVEKKGEANQLSNKFLYPLVDPNSQSGKGTASSGRKPRWYNLKIAGDPSWEEEKKKWEEDKKKDPLAKILGKLAEYGLIPLFIPFTDSNEPIVKEIKWMEKSRNQSVRRLDKDMFIQALERFLSWESWNLKVKEEYEKVEKEHKTLEERIKEDIQAFKSLEQYEKERQEQLLRDTLNTNEYRLSKRGLRGWREIIQKWLKMDENEPSEKYLEVFKDYQRKHPREAGDYSVYEFLSKKENHFIWRNHPEYPYLYATFCEIDKKKKDAKQQATFTLADPINHPLWVRFEERSGSNLNKYRILTEQLHTEKLKKKLTVQLDRLIYPTESGGWEEKGKVDIVLLPSRQFYNQIFLDIEEKGKHAFTYKDESIKFPLKGTLGGARVQFDRDHLRRYPHKVESGNVGRIYFNMTVNIEPTESPVSKSLKIHRDDFPKFVNFKPKELTEWIKDSKGKKLKSGIESLEIGLRVMSIDLGQRQAAAASIFEVVDQKPDIEGKLFFPIKGTELYAVHRASFNIKLPGETLVKSREVLRKAREDNLKLMNQKLNFLRNVLHFQQFEDITEREKRVTKWISRQENSDVPLVYQDELIQIRELMYKPYKDWVAFLKQLHKRLEVEIGKEVKHWRKSLSDGRKGLYGISLKNIDEIDRTRKFLLRWSLRPTEPGEVRRLEPGQRFAIDQLNHLNALKEDRLKKMANTIIMHALGYCYDVRKKKWQAKNPACQIILFEDLSNYNPYEERSRFENSKLMKWSRREIPRQVALQGEIYGLQVGEVGAQFSSRFHAKTGSPGIRCSVVTKEKLQDNRFFKNLQREGRLTLDKIAVLKEGDLYPDKGGEKFISLSKDRKLVTTHADINAAQNLQKRFWTRTHGFYKVYCKAYQVDGQTVYIPESKDQKQKIIEEFGEGYFILKDGVYEWGNAGKLKIKKGSSKQSSSELVDSDILKDSFDLASELKGEKLMLYRDPSGNVFPSDKWMAAGVFFGKLERILISKLTNQYSISTIEDDSSKQSM

SEQ ID NO: 34 - protein, 1468 aa, unidentified
  source 1..1468: note = Description of Unknown: cas12b sequence
MATAVDTSTTRAYTLRLSGGNNWRELLWQTHVAVNRGAWVWGDWLLTLRGGLPASLADGDAERRVVLALSWLSVESPASLAPQAHIVAYGSDARDERNRKVTERFRDILRRMGIKQQQEQEWLDACLPALMASIREDAVWVDRSACFAEAQQCYRGLSSEWARKTLFDFLGGEDDYFKPSAKEGASSKAKDFVQKAGRWLSRHWGAGKKSDPRDISTRLGKLAGVDPKAIDGHTGRAALEDLLRTLGSRPAQNADAEKLYRQLKRAVGWKGRPSKGAVALKKIRDAERVPNDLWKEIASTLREEAAVQSSQTSDHAAVPDWRSHWPAEITGLPMPYRVDRDYIWEHGVMLDHALRRVSSAHTWIKRAEAERRRFQQDAAKMGSIPEEARNWLDAFRERRSSSSGATGDYLIRERAINGWDKVVQAWETLGPNSTRDQRIAAARDVQANLDEDEKFGDIQLFAGFGDEHVDDPERCLADDRATCVWRNSSGRADGRILKDYVAATVAEHNQRRFKVPAYRHPDPLRHPVFVDYGKSRWSINYSALTAAQQRRKTTQKLAQAKTDNTRAKLQQQLASTADLRSVTLGVWDGNRIVKISQRWRSKRFWRDLDLDHFGSHPSAAVSRADRLGRVAARQDPGAAVYVAKVFEQQDWNGRLQVPRRELNRLADVVYGKGADPDFGKLERLDPRARRLWERLSWFLTTSATVQPQGPWLDYVAAGLPSGIQYTKSRAGYYLNYDANHGRKGRARLCLARLPGLRVLSLDLGHRYAAACAVWQTLTIEQMTNECRQAAHPAPSNDDLFIHLRHPTHKPQKSGRKKGRPVTKTTIYRRIGPDKLPDGTDHPAPWARLERQFLIKLQGEDRPARYASQKEIDEVNQFRNFVGLEPIVDRPRVDDLHSDAVRVARLGLRRLADAARIAFAMTAAKKPISGGHEVELTTAQRIEFLQDALLLWQSLAASRRYRDDWAEKLWQSWVVEKLGGPQPAEIADDLPRSQRAASLKTARQSLRKVAEKLSDGQSPSAAELHRLWAERWQQRQTEWRRHLRWLRRLILPRRKDHQQEDRPLQRVGGLSVKRIQTIRQLYQVLKAFRMRPEPSDLRKNIPAPGDRSLASFGRRILNHLERLREQRIKQLASRVVEAALGAGRISKPPGRDRRRPQQPVDRPCHAVVIENLQHYKPEDSRLRRENRQLMDWQARNLRKYIVEGCELHGLLFVEVSPAYTSRQDSRTGAPGLRCEDVSRTALQEAARRMHASHSRPSNSSPGGSQTQFEREVCRWINEFKRVEGSSSSLSARQAVLKAFLHHQASIPTSLSTILLPRRGGELFVSADPDSPLACGLQADLNAAANIGLKALTDPDWMGAWWFVLVDRASGQPVEEQVQGCPIWLSCGPLSNSNPATIDPSDSPTAARRSNGTGAKGRARANEYWWSSLSATTLPDHKAWQPTQDYWRDIEQRVVKRLLRLLDGSEWSED

SEQ ID NO: 35 - protein, 1375 aa, unidentified
  source 1..1375: note = Description of Unknown: cas12b sequence
MNRIYQGRVSKIEIKDSEGNFRNVPVGSPDTCPLWRHHRIFQDAVNYYLVALGALAGTGSENAFVGLGSKDRVIHDLYSRLFDSWERFPRDMHGASSLRDSLRRTLPGLSERASLQDAFDAILSGNEANARERVLSLLSLIQDLGGDIQKGSKRYFPFFCEPATKATFPRARVGLLKVEGKDFVPRLLWSSDLEIAPDQVVEQLKFEYFANPNESVQPIEGNEARVRLIEALDNPQLGIELPIEILSDLRKRVHLIETDIRIPRYFFGGAGAELRKFRLDLFLIAAYVTPDPSILRALRNSFKEPSASKSSKKKDETEEVENLLRSLGDDPLILARGERGFVFPSFTSLPTWVGANAQKPIWRDFDIAAFAEALKSLNQFTAKTEEREEKLKKAEETLHYMLGISDAIPRSSDSETEEQAPSRPGKDPRWPLVAQLEKELGENLSEGTWQLSRSAMRGLRDIIGLWRKHPGASVVTLQKDVKTYQADEKHKREIGSVQLFLLLCEERYHALWQTETDDERGDESEENDDPARILSDAIEVHQIRREVERFREPIRLTPAEPVFSRRLFMFSDLTDKLAKVKFGETTEENSEVKSQFVEAAIALKEGENLKEARVRITFSAPRLHRDELLGGAESRWLQPITAALGFSNPAPSVKFDSAVALMPDHMDDGRIRHLLNFPVNFDSAWLHQSIGKADLWKSQFNGTKDKNLHLHWAGTARDTTRKNTWWENRTIIENGFTVLSNDLGQRSAGAWALLKVTCSRPDTKHPVRSIGHDGTREWFATVLATGIHRLPGEDQRILKNGKWATEQSGKKGRNATFSEYEAACVLAKNLGCESVENWLGMSGEKSYPALNDQLVKIANRRITRLGTYHRWSCFSPEKFEDPARRANVIGGQLAELSAYQDENVTVSADILKSGDFEGFRHRAGAAFEALRTELEVHLVNLANLTAPLRQKVWSWQKRPDSSGYGDLLMVDLDDCHPKIRGQRGLSMARLEQLEGLRRLFLRYNRSLDRSPGIPAKFGREDVGRTSGEPCQALLVKIDRMKEQRVNQTAHLILAQALGVRLCPHRIEENERKSRDLHGEYEKIPGREPVDFIVIEDLSRYLSSQGRAPSENSRLMKWAHRAVRDKLKMLAEEPFGIPVVETVPAYSSRFHALNGQAGSRLHELHELEAYQQQSLINLAAKTDFQNRDRSKAAGELFEQFQALAKLNERRRAEGKKVPRTLYYPKSGGPLFLASRDGDTIHADVNAAINLGLRAIAAPACIDIHRRLRATKEKEVYRPRVGNAREKSAFSKDDIIQPSGAPSKKFASSSSPNFFYEPEDLKQANGEPLFDRAMFGEYSLVSGVSLWSMVNNAIYIRCVELNRTRLHGKDPDDQIPM

SEQ ID NO: 36 - protein, 1254 aa, unidentified
  source 1..1254: note = Description of Unknown: cas12b sequence
MSITRSIKVKLIVPRDASLEARQLREGLWATHLFVNDGCHYYERLLLEFRQRDVCVGKDDAGKDVIVPAAEWADRLRARLGRNGMVPSHIEAALPIFRELYENMVPSALKAKSGTGQAGRSWHSKLVSPTSRGGEASAARIDVLRPLLPVSGDDPAFEPAARALIEEAGDELLTSTGRCPAWVTAYRKGPEGSAWVEKLRIQLREAVEAGDFDPPSDPQILAAGAVPAAPPLGAGIDALRPLLPLLGGDPAFEPAARALVEDIGDELFTSTGRPPTWVTAHPTWVRAHRKDAECLEAADDFKWVERLRQRLRDDAKAGKFEQPLHERLGALGALPVAKPIGAGRVVSRADLTVFERGAMELAIEHLIGWESAGHRARAQYVERKKRHDDLLQWIEAEAPDALLAVRAYEAARTIHLATLGELGAAPQYTLRLREIRPWRKLREWLLQNPDATIDERRRRLATMQTNDPRGYGGEALAWLAAPERRALVEHPAGDVVTRIAVLNIRKSILDRSRLFPTCTLADPVEHPRFAKFGKPGDKNSAGYALAVDGVRREAIIKILVPRQDGLLVPTDLRVPFAPSGQMRDLRASGLDISYERQDGRGRQAAKLQGGNLMFDRTHFARCGAPGPEALGSVWIKVALDLSSPAASLAMKTATPVRTYLSTAVRGRPESTKYEKAAPPEGFRVLSVHMGLRTAATASMLRFGAPEEGGHEVPVSGLAGETLVAFHERTVTMKLPGEDPDTRTEANRGVAKRELRGLGRGIGCLKAIRRASASATPEDRAEALVIIETHVGQGDRHGWAPAEAVGRLDPHGDPDDWKTACAALYAAVEADLGVAISSWRKAARAGGATGMLGGKSLWAVDHLERSFRFLRSWDLRARPHDGDPRRPRPGYASKLLHHIDGVKDDRVKTTADRIVQAACGRAWIGGPTVKRGTQDVRLPGRWEQRGPRADLILLPDLTHFRFRSDRPRAENSRLMRWAHRQLAIYVRMQAEVEGILVADTGAAFTTRFDAWTGAPGVRCEPVTADHLRGIAKREDYWLARLLREGALKHLRIDPASLRVDDLVPMDHGKILVALDGVDLPGLRILDTDVNASQGLGRRYIEGHGLAYRLPGARVPRGEGEREAAVVHIKGKRLASAMGGTVVVLRASEGPGDITWTAEVYDRPQGARKALGLSLAAFNSIATAAVDDEGPAPENDDEALEEEAEEALGIATGERIVFFRDPSGAVAGGGWLEASAFWGIANRMVTDRLRELGRLG

SEQ ID NO: 37 - protein, 1388 aa, unidentified
  source 1..1388: note = Description of Unknown: cas12b sequence
MSLNRIYQGRVAAVETGTALAKGNVEWMPAAGGDEVLWQHHELFQAAINYYLVALLALADKNNPVLGPLISQMDNPQSPYHVWGSFRRQGRQRTGLSQAVAPYITPGNNAPTLDEVFRSILAGNPTDRATLDAALMQLLKACDGAGAIQQEGRSYWPKFCDPDSTANFAGDPAMLRREQHRLLLPQVLHDPAITHDSPALGSFDTYSIATPDTRTPQLTGPKARARLEQAITLWRVRLPESAADFDRLASSLKKIPDDDSRLNLQGYVGSSAKGEVQARLFALLLFRHLERSSFTLGLLRSATPPPKNAETPPPAGVPLPAASAADPVRIARGKRSFVFRAFTSLPCWHGGDNIHPTWKSFDIAAFKYALTVINQIEEKTKERQKECAELETDFDYMHGRLAKIPVKYTTGEAEPPPILANDLRIPLLRELLQNIKVDTALTDGEAVSYGLQRRTIRGFRELRRIWRGHAPAGTVFSSELKEKLAGELRQFQTDNSTTIGSVQLFNELIQNPKYWPIWQAPDVETARQWADAGFADDPLAALVQEAELQEDIDALKAPVKLTPADPEYSRRQYDFNAVSKFGAGSRSANRHEPGQTERGHNTFTTEIAARNAADGNRWRATHVRIHYSAPRLLRDGLRRPDTDGNEALEAVPWLQPMMEALAPLPTLPQDLTGMPVFLMPDVTLSGERRILLNLPVTLEPAALVEQLGNAGRWQNQFFGSREDPFALRWPADGAVKTAKGKTHIPWHQDRDHFTVLGVDLGTRDAGALALLNVTAQKPAKPVHRIIGEADGRTWYASLADARMIRLPGEDARLFVRGKLVQEPYGERGRNASLLEWEDARNIILRLGQNPDELLGADPRRHSYPEINDKLLVALRRAQARLARLQNRSWRLRDLAESDKALDEIHAERAGEKPSPLPPLARDDAIKSTDEALLSQRDIIRRSFVQIANLILPLRGRRWEWRPHVEVPDCHILAQSDPGTDDTKRLVAGQRGISHERIEQIEELRRRCQSLNRALRHKPGERPVLGRPAKGEEIADPCPALLEKINRLRDQRVDQTAHAILAAALGVRLRAPSKDRAERRHRDIHGEYERFRAPADFVVIENLSRYLSSQDRARSENTRLMQWCHRQIVQKLRQLCETYGIPVLAVPAAYSSRFSSRDGSAGFRAVHLTPDHRHRMPWSRILARLKAHEEDGKRLEKTVLDEARAVRGLFDRLDRFNAGHVPGKPWRTLLAPLPGGPVFVPLGDATPMQADLNAAINIALRGIAAPDRHDIHHRLRAENKKRILSLRLGTQREKARWPGGAPAVTLSTPNNGASPEDSDALPERVSNLFVDIAGVANFERVTIEGVSQKFATGRGLWASVKQRAWNRVARLNETVTDNNRNEEEDDIPM

SEQ ID NO: 38 - protein, 1172 aa, unidentified
  source 1..1172: note = Description of Unknown: cas12b sequence
MPTRTINLKLQISPKTDEGRKIRSALWTTHSEINKAVAEIEKLLLLCRGEKYYTTNSKDEEVEVPEPQVKTDALEMARAVQAKNGKAGTGSDEEVLSALRMLYEATVPSSVLDDKGKPLSGDAQSIGGSYAGPICDPETCRIKDVDRLFESGPFAETASKKFTQLPAWFNEVTKKNFNKDEPEKFVKVGKDKDEKFYEIDLRQADAWYESPEVKDIVSKNKAFNKDKWWKNKRDGVDTWAAEFVKKQFDLRKDVRVSIREELWDRLGLLPLGSLYFKKPVGNKWNRMAFRLAIAHLLSWESWNHQTLAEYTKYTKYKDGLIELAGASRSLEVRFEPLRQYQKERHEELSRTSFVDDDRPFTIGARMIRAWGRVREAWRNKGDGIDERRQILADLQTELKGKFGDPHLFLWLAEAGRESLWRDEDVLTTFVEINIAQRDLERHRPYSLMTFADARLHPRWAMYEALGGTNLRNYELTPEGKVKIPLLICEKDKLSEKTFTIPLAPSGQLKSLEIKSLPKKKVKISYASAHQFYAGIPGGSEILFDRLFMENRASSALANGSCGPAWLKLTVDVESKAPPEWLDKKGRVQTPPTVHHFKTGLANKSKHTDKLEPSLRVLSVDLGLRTFASCSVFELVDEKPAKGLFFETDHPHLWAKHERSFKLTLPGEEAGDDPKVAQARREAMDEVYSLRRDMYRLKDILRLKIISAPNERREKLESKIAEMREKQDARAVVTSNFFERLSEKCDLNPPMWEHSCNEIHRDAEKAFSARIGEWRKRTRKRPGSWEEWRETRSYHGGKSYWMIEYLEAVRKLLIGWSTHGRDYGEINRQNKKRYGTVASKLLKHINKLKEDRTKAGTDLIIQAARGYIPLPGKGWMEKYRPCRVILFEDLARYRFKVDRPRRENSQLMKWGHREIINEATLQGEIYGMVVETAGAGFSSRFHAKTGAPGVRCRYLKEDDFENGAPKEFLVRQMKNLMKGDRLEPGLLVPWDGGELFATVDNGKPIVIHADINAAQNLQRRFWTRFADAYRVNAVEENDNWVVTDTGVRVLGALEMAVHGEADRKPRTGFTLHGTLQSGAELKAEGKKTDIKDVEEDKDDSISSEIIELQDEKERKGRETFFRDPSGGILDPGKWYGSKRFWGRAKGAVTEALLDNQGANNALEEKPGNDELPF

SEQ ID NO: 39 - protein, 1108 aa, unidentified; identical to SEQ ID NO: 5
  source 1..1108: note = Description of Unknown: cas12b sequence

SEQ ID NO: 40 - protein, 1450 aa, unidentified
  source 1..1450: note = Description of Unknown: cas12b sequence
MYRGFCTVTATSGGWQSTTFLAGAQMADTTTRAYTLKLQGDRLALWRNHVIFNNGVKAWGEWLLCLRGGLPASLADHRDSLDVSKGEISRTFKERTAAITPATIRQELKFKAATEKKVREEVASRRKKVTETAVAKELLAARRSELRRILALSWLCPETPVQLVPQAAIVAAADDSDREQKVLDGFRQILKRKGVSDVAGWVQDCDATLRATIRSDAVWVDRTACFCSMPRAVRPSEVDAAKHLFRLFGSMSDYFATASASSGPAEPKDFANTCRDWVSSFWGGGEKSNKASILAALSAIAQIKPTRVVGKRGPAALAVIAGVLEQKPVDDSVEALARAIGWLSGRPSAARLAINAIAASPRVSQKLWDRLVLACEKDCGRQKSKLAFEGSASTIASALEPRLAGLTGMPYASTGRELIGEYATMLAFAMRRVSQIHTKAKQAEAERRSFAPEQARLALVPSAARKWLEDYVEARTAASGAVDGYQLRKRALGGWADVVAAWSRCETSEDRIAAVRELQADWEKAGDVQLFEALAADDAICVWQSANGKTAASILTDYVRAAVADQNATRFKVPAYRHPDPLRSPTFVGFGNSQWSIAYSAQGEARERRKLLDRASGSAKDAERAREGLAREAVLQNVSLDLWAGDKMVPTQFRWQSRRLLSDLALHSVPAMKGAKVTRATRFGRARIAAGPVLLDGIADDTPWNGRLQAPRRQLEDLARILDAKGLPFDDESKWPPKVRSRLKHLGWFLTHSAKLTPSGPWLDYVAGGLANGWKWAEGREGACLFREDNKDRKGRAKLILSRLPGLRLLSVDLGLRTSAAAAVWQVVSKRQLTAAKDGAKSVSDTDLFCLVRTGDRTQVYRRIGLSAWARLERQFLIRLDGEKAAARPATTNEWESLQSFRAWLGCGIERRPEKLPPVDSLQQSAERLCRLGLRRLSDLARVAYLLTAKERPIMGGRTAPLDEEGTVQAAQDALSILHALGSSEDFSDARLQGIWRTAIGDTPPLAARLTKKQRQELREALRPAAEKLRGKAALGKELADLWKERSAAWAKHLRWLRDWVIPRFDKRKNGERVRSARGVGGLSLDRIATIRGVYQIMRAYASRAEPTNLRAGVERLEKAAAKKLRPEFGRRMLAKMERLRENRVKQIASRIVEAALGVGSEDRLHWERGRRRPTAAISDPRFAPCHAVVIENLENYRPDEKRTRRENRGLMSWAARAVGKYLAEGCQLHGLYLRQVSPAYTSRQDSRTGCPGLRCNDVRAQELLNPEGWIGRLVARAAEAVKEGKATPRQRLLVTLAESARAGIAESAAVRIIAPGGQLFIAADPQSPASNGIHADMNAAANIGLVALLDPDWPAAWWRLPCKAATGYVDESKVGGSEAVPLGRAILEVGAEAGKVYVNAWSDPQDSAVSRREWTDTKRYWRDVEERVVEILLASNRGGRRGKPGAVPF

SEQ ID NO: 41 - protein, 792 aa, unidentified
  source 1..792: note = Description of Unknown: cas12b sequence
MATKSFEAKIVCKPDEKYTAEQKKQFLWFTHQVFNDGVRKVIPYVFKMKRGELGPEFQAIYYAITSSQDAIGKLEAVINPDWTSGKIGKSDPNKWKELLKYQELEKGFRQRLKEEGIKSTKKFRKELEDEKKKLAKEIGQKDIWADAAAILRNKNLLLFNRDELLPNLPSEFRRKIYEMTIQLIHGHQELVANWEDEHAEWLIEKDKWEEEHPEYMNVRPIFEKFEKEQGKVKGSRIRWLAYLDFLSSKPELANWRGKAKETIPLTKEERAGFRKPGQHFAAFFNKNPELQELDRLHKEYQEKFARTQSKRTPHPDGFKHRPTFTLPDAMRHPVWYSFKGATDPTKGSTYRNLDLENCTLDLKVLTAMEGEGRNPGGMIQYAFEPDERIKGFRYVGTTEKGKRAKGYIYYDPILEKERPAKIQGIKLVFRPPRPDGTAYLIFSCQIEDEKPKIKIWKDKEEESPGEITKRKKTEVYPPELITLAIDFGQRHLGAITICKNNNGRPEPIRFIPAYPKRRKDRESKPVSAWLAKIPGLTFNAVGMHEKEISAGMSRRFQDPKSIRQAGEKEGRKSKGQHIPETETPWAHLREHIANMKEDHYKKAANLIIRTALQNGAQVILIENLRNYRPMLERTNLENRRRMQWAVRQTAKFLEDTARPLGLIVRQVSSAYTSRFCSSCGHPGARVSLPGQKNWEKFYAEKYGKERKMIAVAGGQFFCCPACKKIINADINASLNMHKVFYQNFIWPGKIDKKDTKNFIWQGKNYNWDQIADDVQSFLDQKAGIKKEDDIPY

SEQ ID NO: 42 - protein, 1382 aa, unidentified
  source 1..1382: note = Description of Unknown: cas12b sequence
MAYGSEAPDERNRKVTERFRIILSRMGINQQQEQEWLDACRPALTASIREDAVWIDRSACFAEAQQHYPGLSSEWARETLFDFLGGENDYFALPDPEAAPSSEAKDFVQKAGGWLSRHWGAGKKSDSTAISTNLNRLAGVESKAIVGRCGCDALAVLLTTLGGWPAKNADSGTLYHQLKQAVGWKGRPSRAAKALEKVRDAPEVTDALWRQTADTLRQEAVAQSSRAAGGSGVPAWMPAWREDMEARLGMPYRGARDYIWEHSVMLDHALRRVSSAHTWIKRAEAKRRRFQQDADKIGSIPAKAREWLDAFRERRFSASGALRGYLIRERAIDGWDRVVQAWASLGPNCTREQRIAAARDVQANLDEDEKFGDIQLFAGVGDEDEGDPQPCLADDDAICVWRDLNGRADSNILKDYVAATVAKHDQQRFKVPAYRHPDPLRHPVYVDYGNSRWSIEYSALKAAHQRRKTTEKLVQAKTDRARAKFQQKPADTPDLRGVTLGVWTGSSIEKVSLHWHGKRFWKDLDLDHFGRDPSATVSRADRLGRVAASQHPEAAVHVAKVFEQQDWNGRLQVPRHELQRLADLVYGKGGDPDFAKLGSLDERRTRRQWEHLSWFLTTSTTIQPRGPWLDYVAQGLPQGIQYKKGRNGYYLEYAANQGRKRRARLCLARLPGLRVLSLDLGDRYAAACAVWETLTREQITQECHQAGHPGPSQDDLFIHLRHRTGKPQKSGRNKGKPVTKTTIYRRIGPDLLPDGTPHPAPWARLQRQFLIRLQGEDRPARFASQHEIDGSNRFREFLGLPPLADRPRVDDLHRDMVRLARLGLRRLADAARIAFAMTATKKPISGGREETLATEQRIEFLQDALVRWQALAASSRYRDDWARQAWQEWIVEKLGGPQPAEIADELPRSQQATRVETARRSLREVAAKLSNPQSSSATELHGLWAARWQERQTKWRQYLRWLRRLILPRRKDYQQANRQVHRVGGLSVKRLQTIRQLYQVLKAFRMRPEPSDLRKNIPAPGDPSLASFGRRILHHRERLRQQRIKQLASRLVEAALGAGRISKRLGRDRRRPRQSVDAPCHAVVIENLERYKPEDSRLRRENRQLMNWQARNLRKYIVEGCELHGLLFVEVWPAYTSRQDTRTGAPGVRCEDVPRSVLEEATRRIRALGSAPSGSSRGRSETRFEREVCRWIHEFNRVVGSSSGLSPRQSVLKAFLDHQAAIPTWRSTVRLPRRGGELFVSADANSPLANGLQADLNAAANIGLKALTDPDWMGAWWFVLVKRDSGQPVPQQVQGSPIWESCTRLSSPATVDSSDSPAGARRSKGRGARGRARATEYRWSPLSAMTMPDNKTWWPTRDYWPEIERQIADRLLREQIDPENRF

SEQ ID NO: 43 - protein, 1272 aa, unidentified
  source 1..1272: note = Description of Unknown: cas12c sequence
MKKTSPLKRSALRTARRQIARGCLPIGNRDISTTRTRVLPLADSVADAVWNQARTAALTLRGFGSGSLFDLLLDLHASGLRLFSSNGEREGFLLKQKFDAGKFDRAAAKDVGEDMPKFTAANLRAALVAIPRGGGPDTDAKALATRLARAVGVKATKLDKPPKLLKDMAKELAMAFPTWKELSTANGEVGAVIDDVARMYGLRWPSLRRGWAFRLPEVTRELGSPTLAFDPDAPVIDETSATARFAAIVARYLPECGGLTDSAAAKGVQARITTTNANGLSWLFGVGLRGMRDLPVDTVADTLAIDVTRGRDALRALVNDIKALPRLGEFGDRVYVESRATLQGAVDSLIANYVGRLADLVASADALEHDQPRPPVLDDADWKPAIFDGMGFTPWEVEDMLDARPVEVARLRLALGVLAGTTPAVAGDFARALADVEAFGAWAARTEAVAALINARVKVLKAPESLRLRGVLGGGRWKAVVSIHPDEGEPAQVIPQLDTQLQALLDDGQRAFDVLVADYTPTFAAALEHARSDMRASLADKGREAPSAESIDLLARRKLLDMVARVTRRGSPSLGHAFLAACAVQGLTRPGTATERSLRGHILSGEQALFVHPYARARSIVRLEHAGLLRLDLDALLTAMERDAEQRADVREQIVLRFTRQSLLLGGLPGRIRLAKVPWTQEAAAASGVRGAPWLKLHPDDAGTVARSEVIKAFTARFHLSANGLLYRLNRMRFLERYDIRCFIGDTLLFAPKAGAWTPPEQYRHGKYAHWLSHPDLPRTEGGAVDVVPAARWLTEASRRADEDGRASAVALLAQFPHEWVAACEFEGAPVYEGVFPCEGKIGGWMKRRGYRLAPPRHFAGELLAAFKDASVSPHGLTFEREMLREGTTVRELSRRVVAAYPIAVPTHPDAERPWSPLHLMGLDLGEAGLGVCLRHIGTGAETTLLLPVRKTRLLAHREEHYRRKVQPRQAFRKGYGDAMELAVKAAIGEVCGIIDNLIVRYRAVPVFESAVAQARGSNKMIQRVFAGVVQHYTFVANNGAAQTVRQSHWFGAGRWSYTYGADLLPAARQMTEKQLLKAKAEAVFRPAMGFPGVMASGYRTSLVCACCGEDVLDAVDAAAEGGQVALTTDAEGSGVLDLGGRSLRIKLEAPSPNPIVQKAARRKRRRTPWEALADRTWTLTHKTDRADLVATLRRGLRRPPASVQGHATSGWEFHCAACGHIAQADVNAATNLVRRYDDRVRKMEQARAHWDDPSVRAKLASELAERAAARS

SEQ ID NO: 44 - protein, 1262 aa, unidentified
  source 1..1262: note = Description of Unknown: cas12c sequence
MLTTKFKLELPAGCPLREDAATFDECRKLYDVVEGCGNGTLTGFLFSVILSGFRIFPDGKTAEIFANRSVYDEDEFRSALVEAVGAPLPRFTVKALIKRLQMEVRARGNKDNRFVAEVMMKEYRQTLCGKTLPKGVDESYVDRLFEEMARELTSRYRSWNELKGDLLGACKAVDAALRGFGDFPSLATMVTRAAARRLPKDSTIVFDPQSPCIDVQTIGVDAMPYAAVSTILSYPESVGEKRRDFVQNHLTTPSAAGLSWLFNRGLELFSEESVEELCRLFHVPEDQRTRIVQIQNAARATPRQSFFLKKGGAPLGYHDFRSAFAGRINSWTANYLNRLEELQGLLHDLTDELRLPDLVRNGEDFLATTDCRREEVEILCRSFSRERDRAQTAVEHLIGADPLQVVSDVAAIEEYSRIVNRLCAIKEQIVNSLRQAEDDKASRWTALWSEVKDEFQPWEKLIRLPKLNGMSGGVPPAQDELETILARYSDVGRGASEHFDAVMEWAAKTGAEGDVLKKFAETEQQRADQRAPGKYDGRELALRLVLQRVARVVRDRSDACAENVRQWFLKENVFAERKDFNKFFFNRLGNLYVSPFSNRRHAGYKLSDGLVERSGAVWRELLALVKEMRGAYASFSEAGETFLRLESLLMGMRIGALTKNIPAEVAALRLDDETALESVSEGLKLQLQQAEVPPSVLAKAFNVYVSLLSGCLIALRRERFFLRTKFSFVGNTALVYVPKEKSWPMPSRYEASPSWTPIFENDVLVRLSTGEVEVAETFRRAVALWGRTTDPVLKKALRELFHQLPHDWCCQVSVRSSGDMTPAKRKEDDRDVLIVEKKGKYDSTIISKKIAATALVRLVGPSTHKERLNRLLLDVGEVACDMTLLADQEILQKVEDDRVHLSPGKLQFSLSVPISTPAEQCEDEVKSERKSTHFRRIVAIDQGERGFAFAVFRLEDAGKKGAQPIAQGFVNIPSIRRLIARVHSYRKGKQSVQKFSQRFDSTMFTLRENVAGDVCGAIAGLMSRYRAFPVLERQVSNLASGGKQLELVYKMVNARFLDDRIPMHSLERTSWWCGTSDWVIPDLWVEVPESYAVKAKKDEILEKDGKFYRTLRITPGAGVNAKWTSRICSQCGGNAMELIEKAREEKVKTVTLDANGEVTLFGRTLRLYKRPSEERSREARRRNERAPWTEPRADVRLSLDDFRRAVAENMRRQPKSLQSRDTSQSRYFCVFTDCRCHNKEQHADINAAVNIGRRFLESLLRE

SEQ ID NO: 45 - protein, 1262 aa, unidentified
  source 1..1262: note = Description of Unknown: cas12c sequence
MRRQHHGGQNARDWRRKVAAAALRQKESVFTYKFGLSVNDGDFDFDAAARTYDITEGIERGSLIGLVCAVHLSGFRLFSKVAETRQFLNRSRYPENEFAQALAAHTEIENPSVTVQSIESVFVTPPRKQDGVARLWSADELAKRLFQTWNNRSPREGERNHPELLLAQGIARAVTKAFSGWKELADNAVHALTCADNYLATLGNRFPKLSDLPPLTAGSTQTGTLAFDPESPFLNMTGNEDIWLHQVVAVCAGRLKRYMPEIDPSSRKFASRLTDSIVSSQNNGLSWLFGNGLRFLRQSSIAQIAETLSVSQNEHRRVEQLKEFADAIPVNPFFATDGYAEFRGSVGGKISSWVSNYWKRICELTVLHSQPPDITIPEGLLASENATLFSGQHTAAAGLVALSARLPSQVRDAGKALFVLSGDGVPRADDIATVEDVAGELAELTGQLAMLDNRIQQEIERAQDANDEGRVGSLASLRPNPTKELKEPPKLNRISGGTADAAGELARLETSLNDLIRARREHFYRLAEWTGNTASLDPLPALAERERKALTDRGMDPTLAEADEYALRRLLHRIAGMARRLSPNEAKRVRETMTPLFLKKREANLYFHNRAGALYRHPFSNSRHQPYSIDLNRARATDWLAWLEERAREMLGLLGSGAPANHEYLRDLLSIETFVFTTRLSGLPAQVPGYLAKPKSDLTNIPPLLAAQLDVDEVSRDVALRAFNLFNSAINGLSFRAFRDSFIVRTKFLRLGHDELFYVPKARAWKPPADYRSAKGKISKGLALPAVKRNEAGSILPRETTQGLSRAKFPEGSHALLSQAPHDWFVELDLRHDKMPQLAGLPVKMNADGLKGWRARRRPTFRLAGPPSFKTWLDRALTSTAVKLGDYTLILDQSFKQSLRVEDGEVRLSAEPAGIKAEIAVPVIDARPFPETEAEALFDNIIGIDLGERRIGYAVFSLPALLKSGNPTRVKPTVVGSVAIPAFRRLMAAVRRHRGSRQPNQKVSQTYSTALQQFRENVVGDVCNRIDTLCERYRAFPVLESSVANFETGANQLKLIYGTVLRRYTFSNVDAHKSARSAYWYSANRWQHPYLFVREWNKAQRTFTGSAKPLAIYPGVTIHPAGTSQICHRCGRNALRALRNMPDRTIRVGKDGLIVLADSTIRLLERADYSDRELKTFKRRKQRPPLNMPVPEGARPRDQLERVLRRNMRQQPQSEMSPDTTQARFTCVYTDCGFEGHADENAAVNIGRRFLERIDIEASSRT

SEQ ID NO: 46 - protein, 1283 aa, unidentified
  source 1..1283: note = Description of Unknown: cas12c sequence
MTHAKKIPFPVLKRSTLRKARQRIAAGSITAGERPFNSTVTRVVPVKDPVSDQVWAVAREAAMTLRGFGQGSLFDMLIHLHADGFRLFPSGREREAFFLKDLFDPTEFDDGARRAFGDVMPGFTANSLREILGAPARKCGKVTSVEILLPRLSKGLGVKKSAAPPEVLSSLAAALCEAFPTWSLLTAVDGGVGKVIDDVLRTHGSRLPSLEKAWSTNLPEVPKGLGVPTLAFDDQAPAQSEQTPTGRFAGVVARYLAETFASNPEATAGDASKAVQAKVTTPNGNALSWLFAVGRRAMCSTTLDELAIGLNITSPRGRHALSSLKERMMALPALSVLGERAYPDSRATLQGTVDSLIANYVNRLFELSSSATSIAQTKLILPAAIQGDTAVFDGMPFSAEDVGALFEQLPSEIAKLEHAVKVLVGKERTSTLGYQKAVDDVDEFGVWASSVDAVIGQINARLKTLERAQEPLGKLMGDGKLKRLVNIHEPEGPAVEIIPVLDQELQDVLTSCRTAFADLEARYPMTVAKAQRHAEAEVRNALELASRKEGGLSLASADVPALAKRKILEPIISIARRSSPAMATAVLTECLRQKLIVKGTGSERSLRGYVLSGEQVIYAHPLSRRRSIVRLDREGLQNFDALEFLDALQKDATQRTNVRESLIVEMARQSLLLSALPDRIEIGAISWQTPSQNQHAPWANLRPVNGTVGRSETIKSFTAVFHSRISGLLYRLNRQKFMEKYDLRCFIGSTLLFSPKNADWAPPPQYRHGRFSALLARSDFPWEGAEGTHANAVRLAKFLIDETRNATDLQQAIAAKALLAQLPHDWVVCCDFDGAPSYEGAFVSAGEVSAWAKRSGYLLTPPRHFAGAFLEGFKSTKISPHGLTFERMLERDGDSVIETGRRVTAAFPITQEVAPAAQPWKPRHLAGLDLGEAGLGVCLKNLDNGHEQTLLLKTRKTRLLAHSAEHYRRKDQPRQVFRKQYNQSSENAIKAAIGEVCGLIDNLIARYDAVPVFESQAAAARGSNRMVARVYAGVLQRYTYVVGNGAADATRTSHWLGANRWSYSFGADVIPKVRDLSPEVLRSIKKPENVFRDALGFPGVLANAWRTSMICSVCGTDPIGALEEAIAANQISFVTDNEGEGSLDLGDGRKVTLRVEVPTSSALTKREASRRKRRAPWEAKVGTVWTLTRKSHRDDLLTTIRRSLRRPSSTFQGSTTKQWEFHCPCCGQIQQADVNAASNLVRRYFVRASDNARARQHWADDSKRLAFIASMGPDRSAREEKVS

SEQ ID NO: 47 - protein, 1125 aa, unidentified
  source 1..1125: note = Description of Unknown: cas12d sequence
MRKKLFKGYILHNKRLVYTGKAAIRSIKYPLVAPNKTALNNLSEKIIYDYEHLFGPLNVASYARNSNRYSLVDFWIDSLRAGVIWQSKSTSLIDLISKLEGSKSPSEKIFEQIDFELKNKLDKEQFKDIILLNTGIRSSSNVRSLRGRFLKCFKEEFRDTEEVIACVDKWSKDLIVEGKSILVSKQFLYWEEEFGIKIFPHFKDNHDLPKLTFFVEPSLEFSPHLPLANCLERLKKFDISRESLLGLDNNFSAFSNYFNELFNLLSRGEIKKIVTAVLAVSKSWENEPELEKRLHFLSEKAKLLGYPKLTSSWADYRMIIGGKIKSWHSNYTEQLIKVREDLKKHQIALDKLQEDLKKVVDSSLREQIEAQREALLPLLDTMLKEKDFSDDLELYRFILSDFKSLLNGSYQRYIQTEEERKEDRDVTKKYKDLYSNLRNIPRFFGESKKEQFNKFINKSLPTIDVGLKILEDIRNALETVSVRKPPSITEEYVTKQLEKLSRKYKINAFNSNRFKQITEQVLRKYNNGELPKISEVFYRYPRESHVAIRILPVKISNPRKDISYLLDKYQISPDWKNSNPGEVVDLIEIYKLTLGWLLSCNKDFSMDFSSYDLKLFPEAASLIKNFGSCLSGYYLSKMIFNCITSEIKGMITLYTRDKFVVRYVTQMIGSNQKFPLLCLVGEKQTKNFSRNWGVLIEEKGDLGEEKNQEKCLIFKDKTDFAKAKEVEIFKNNIWRIRTSKYQIQFLNRLFKKTKEWDLMNLVLSEPSLVLEEEWGVSWDKDKLLPLLKKEKSCEERLYYSLPLNLVPATDYKEQSAEIEQRNTYLGLDVGEFGVAYAVVRIVRDRIELLSWGFLKDPALRKIRERVQDMKKKQVMAVFSSSSTAVARVREMAIHSLRNQIHSIALAYKAKIIYEISISNFETGGNRMAKIYRSIKVSDVYRESGADTLVSEMIWGKKNKQMGNHISSYATSYTCCNCARTPFELVIDNDKEYEKGGDEFIFNVGDEKKVRGFLQKSLLGKTIKGKEVLKSIKEYARPPIREVLLEGEDVEQLLKRRGNSYIYRCPFCGYKTDADIQAALNIACRGYISDNAKDAVKEGERKLDYILEVRKLWEKNGAVLRSAKFL

SEQ ID NO: 48 - protein, 1183 aa, unidentified
  source 1..1183: note = Description of Unknown: cas13a sequence
MPIVKKFGRSQTSLSDRKIVLKMETAARNIPDFLLSDPEAVIGQWASAMDKIAKKPKGKDKPSSYQRKFRERLGKAIWADLTGPEGPLRDVPAAELEDLRKRWDRRVHPYPDGTKDGPKPATPKGRLYTRFAGEVGYGKADAVAIARDIRIHLLETEFKTGGGTRDAGRAVRRASSIEKNVLKKARVPQRPKPPQEAAWSKEDEDRYFIPHDVARKIVLAAKAQEKEDHRVAWRTAAAVLFEHFGRIFQQDGRALSFAEAEKQMPGLLALHRAVEGYYRQALKRHRKDRREHEARPGREKGTGRRKVSAILPKDKTALLALIGHQHQNREIAALIRLGRILHYEAGRRGNSDMVANINRNWPADVSESHYWTSAGQIEIKRNEAFVRIWRSALSHANRTLGDWLSPDEVANDITMSWESKHEKSGKRKTGKLEENREEAEAHAPVIFGGSAERLGTGDDFQKTLEAICEVFSQLRHSSFHFRGLDGFKDALTKTVKTCDPGAVARLQDLHAEDQANREARLKEDLRGAHAELFLDEGRLAEIWALLHPKSTEKTLPPLPRYSRVVTRAENTCNGLKLPKSVNRESMKVPAIHCRYILTRLLYQSGFRTWIAEAPAAQLNRWIETATERAQKATVGITKNEADRARMVGQIKVPEGQGIRRFLDDLAGLTATEFRVQAGYESDREAARDQAAFLENLNCDVMALAFDKYLSDHKLGWLAGIDAESRPSETPLSNVDELPSSGSLGTPERWEAALYAVCHLIPVSEVGRLLHQLRRWSNGQKATPDGGRLERLFELYLDMHDAKFDGSTPLRDHDDLAVIFETTGIRDRVLPSSLQHGEHERLPLRGLREMLRFGNLRVLAPIFATAKVDQAMIGELEGLEARIGDAPSQVDRAQALRTEMHAALCKKRKLAHDDKKSVKDYLTSLQTVIRHRRLANHVRLTNHVRTNEILMSVMGRLADFSGIWERDLYFVTNALLYQAGLTPCDVFSKEPPKENRRSPLQEFENGQIVFALRKMQAQCDTHAGLVDQIKGETARLFHIAEGAPGNDPRIQNRNWFAHFNALKPKTGDRLDLTADMNRARDLMAYDRKLKNAVVSAIVTLLERENIVIAWTMKDHQLTDAVLAAKSIEHLKQNKIRENLRDERSLGYVAALFGGRVAEEAPDIMHDRTVFHLVGALTEEMEPAE

SEQ ID NO: 49 - protein, 1276 aa, unidentified
  source 1..1276: note = Description of Unknown: cas13a sequence
MRIVRPYGESRTDLGGERGQTRVLVDNTAARARHEIPDFAQSHDALVIAQWISVLDRIATKPQGTQGATRAQHAFRDRLGRAAWAQMCAADRISAAAQADPYVAALWRFKTHPYGDAKYRPRKGKDGKPLGEPKPQGRWYGRFAANAEPEQADVAAIAALMDHHLHVAELRIDPKRPEKRKGLIEARAKSIEGNVLVAEPRKRPVGSWSREAITRYFMRQDVAAEIFAAARDREQGLNDVPRGPVRLALAAKILHGHWTRLFHAPGTRTAYSIREAEEKEPELFALHMAVKDAYAKLLKRRTQPKTLKKGVKPPQQAPVTTVLPKNAGELLRLVQHRSRNRDLSALIRRGKLIHYTAFDIAAAAAEAESKTPDVPDADRLAYVLTHWPDDLSASRYLTSDGQSAIKRSEAFVRVWRHTIAMASLTLRDWASMNNDLGDVLGSANKVDQAIGRANFDPAWHDKKVRLLFGARAALFPSDDDGRKALLASVIRAGLALRNSSFHFTGRGGFLAALKKLGSEEVMVPSILAAAHALWREDATARAGRLRAALTGAHAAHYFEEDQNASILTLLDEAPPKESLPIPRFRRVLGRAENTWKGKEALVLPPTANRRQLEDPARRCRYTILKALYERPFRSWLIARAPEEVNAWIDRAIERTTRAAKDMNAKRGEDDKRSVIAAKAESLPRLSGERGIGDFFFDLSSATASEMRVQRGYGHDGEAAKEQAGYIDDLLCDVVALAFDAWLRNPQANGRPLTFICDLKPETPLPAAPKCTLQEIGSAAEPVRPEDWQAALYLLLHLVPVGEAGRLLHQLAKWTVTSRLADDLLNANVTDDPSKAERTADEEDLKRLVHTLIQHLDMHDAKFEGGDALTGCEPFAALFASRPGFARIFPAEADERLDRRVPKRGLREIMRFGHHGLVASFAEDTRITDKEVGDYLRLEIEERPDNVAALQARKEEAHERWVKAKEKRKTVDPKHLEDYVTALCGIARHRRLASRVTLTDQVQVHRLLMTVLGRLVDFSGMFERDLYFAMLGLLDEKGARPDEVFSGPIDEPKSRLALLANGRVLAALREQIPHSKDLAEELRKDLERLFGMDCSGIRLLEADERGDTCLRDIRNDLSHFNLLHDDSFALDLTTLVNRTRGLMSYDRKLKNAVSKSIKELLAREGLTLSWDMTDRHDLENARIGAKPAVHLGGRKLAFRGGDRRPEPVRENLHSPTHLEAVARLFGGKVVEEDDVTNLDLSSIDWAAEPHNSKETHRHRPAGPRKSPPKRRAYHAPR

SEQ ID NO: 50 - protein, 1225 aa, unidentified
  source 1..1225: note = Description of Unknown: cas13a sequence
MRIIKPYGRTLVEHDGAGERKRVLTLRPDHDSKLDIEAFARDHDELVVAQWVSTIDKIAAKPGPRKGATEEQRAFRDRIGKAAWALLVRNALLPGLADADRADRLAKIWRRKIAPYGDLRPNERPASAKGRWYGAFAGEADVADVDAGEIAAKIHEHLYDAEYRISGDGRKPDGCIAARARSIAVNVLRPADSSACGQPEWSDRDLQAYRVADVAKQIWDAALSRENGRDGAGTKRVTNSVAGGVLFEHWARIFPGPDGKALSIREAIEKEPGLFALHMAVKDCYARILKHHKKKAPGRRERENGDVSPIRKVLPRDMDELFARIISGRGNRDLNALVRLGKVIHYTASDPNADHPESITENWPGDLAGSHYWTSAGQAEIKRNEAFVRVWRHVVVLAARTLTDWGDPHGEIGSDILGKANDATGAKFDEAAFNRKCALLFGKRASHFTAAPDLAFKKAVLKTAIKGMAALRHKSFHFAGRGGFVKALEGIGGLNEIDRFPDVTRALRTLLVEDIEDQSRQVRATMVGAHFGVYLSKGQVEAIYRAVTGAEPGSLPLPRFSRVLRRAKGAWEAEDVLPPPVNRLDLEQRGRLCQYTGLKLLYERPFRRWLEGRSAAKLNGFIYRAVTRASDAARTLNTKESDDWRDIIVARAEKLGKVPDGGDIHGFFFELSAETASEMRVQQAYESDGERARQQAEYIEDLKCDVVGLAYRSFLETEGFDFLRTLDPEAAIAEAHRFDPAELPDPAVDTDAEDWEAVLYFLVHLVPVDEIGRLLHQMRKWDLLAHDRTAPVADGGQARLVDKVQRVFTLYLDLHDAKFEGGEALTGIEPFRKLFEESDGFDTIFPPQQGYEEDRRVPLRGLREIMRFGDLPPLLSIYGRRPATKSNIERYRRAEVADAGGRSEIARLQARREELHAKWVEAKKEGLGPEDRRAYVEALAEIVRHRHLAAHVTLTNHVRLHRLMMAVLGRLADFSGLWERDLYFATLALLHRAGKTPREVFENEGIDLLRNGQIVYALRKLNGSSNASALRSGLFPHFGSAFKRGDPIGGIRNAFAHFNMLRAAQPPNLTECINRARQLMKHDRKLKNAVSKSVIDLLAREGLNIAWAVHTRAGAHDLAEAVLSSRQAQHLGKLRLFPVSGDGRDGKGFFIMEDLHGADFVEMAAELFGGRVSDRWRGKGCVSELRLDSIDWSRQREQKKHGGGKKPTGRARKANRGHKNRHRRA

SEQ ID NO: 51 - protein, 1076 aa, unidentified
  source 1..1076: note = Description of Unknown: cas12f sequence
MSARNIKVKIDTKGNPELRLGLWKTHQVTNEGVKYYTEWLIKLRQQDIYRQSREDASPRVIISASDLKADLLCHARQLQKERLPRITGSDAEILGTLRQVYELIVPSSVGKSGDSKTLARKFLSPLTDPGSAGGRDQSASGRKPTWMKMKSEGNPRWEETFRKWKDRKDNDPTPLVLNQIADYGLLPLIPLFTDVGENIFDPKSKSQFVRTWDRSMFQQAIERLMSWESWNQRVRREWEALNQKHSAFYREQFTADPDAALYRVAQSLEEEMRKEHQGFASDAPEAFRIRRVALKGFDRLLERWQKTLGKNGQSATLLDDIRRVQSDLGDKFGSAPLYQKLLDERWQRLWAVDPTFLQRYAAFNDLTQRLQRAKRVANLTLPDAVAHPIWSRYEGANASSGNRYHIHLPTKGQPGSVTFDRILWPDGNGGWYERKRVTVFLRPSHQVDRIHEAPTDSVVDNFPLVVEDQSARTILRASWGGAKLEYDRNRLPRQLKKGVPDSIYLSLTLNLDTNKPSGLFHTQQNGRVWIRKDVLMQYYNETPGDNVQFKPLYVMSVDLGIRSAAAVSIFSVQLKAGIEEHRLTYPVADCPGLVAVHERSVLLTMPGERREQWDRRYEQQRQGLRELRTDMRGMNDLLRGAYMDGDRREEFLARLSKLEETSPELWGPVYRSLNDSKVASATEWERLVVYCHRQVEQSLSSRIQNLRSGRSAYRMSGGLSLDHVQDLERIRGIIASWTNHPRIPGSVVRWQQGRSHTVALGRHILELKRDRVKKVANYLIMTTLGYAYDSKRARGEKWVRRYPACHLMVFEDLTRYRFRTDRPRSENRQLMRWTHQELIAVTGIQAEPHGISVGTMYAGFSSRFDAVTKAPGVRGATVRQILRTRGMVRLKEIAADVGIDINTLRPHDVLPTGDGEYLLSVVRHGESYRLKQVHADINAAHNLQRRLWTQDEVFRVSCRLALNSGRVVAMPPPSYNKRYGKGFFEKGDNGVYIWKTGGKIKISDTLEEDMDIPEDTAELLRGNSVTLFRDPSGTIAGGNWLEAKEFWGRVNSLVNKGVRDKILGGIPVDNSSAHAE

SEQ ID NO: 52 - protein, 660 aa, unidentified
  source 1..660: note = Description of Unknown: cas12f sequence
MPMIKITECVTWGTTCDGLWDARPHLEVRRSWSPPVQGGRTNRLDAPPASVTLNIHGRVEHPRDADALSVAPLRVRHMFERTTTKAAFLSPLDLRPTQATDLERFAGTTRWAFNWANALLEAHHQAYEGRRQQAARHLFGLGPEQLDELRVLANGTRDENGKKAKGDPVKRREYESIQKATKKAVSEENKALGAEMKLWDEHRSLVVHKGRPLLTPGDEPALDAPPLAHRLYARRVELAGIQKTDPDYYAEQRKKEREAITPNVVAMKRDLMAKGAYFPSEYDLQYIWRTVRDLPKEEGGSPWWPECPTILFYDGINRARTAWKNWMDSASGARKGPPVGMPRFKSKYKAKDTFTITNPNRSVIKFETYRRIAITGIGSMRLHRGAKLLARRIAAGQAEITSATISRSGTAWYVSVLCTVHTTARTAPSKAQRSRGAVGVDWGVRALATTSKPIALTPGKPASRTVPAEKYGAAMSQKIARAQRQLARMPKGSSRRRKAARHVADLQHLVAQRRASSVHQLSKALAQSFEIVAIEGLNVRGMTKSAKGTVENPGKNIRQKAGLNRAILDATPGELKRQLEYKTKKYGSRLVELDTWYPSSKTCSRCGWVHPKLKLSMRTFRCQQCGLVEDRDFNAAVNIERQGITHIVKENEGTDDREEG

SEQ ID NO: 53 - protein, 696 aa, unidentified
  source 1..696: note = Description of Unknown: cas12f sequence
MSTPMGWTAVNGGDATSPTTRVSSPPGEPRTGACPRAAAADATRAESSPRRTSSPARPGERHARARTSRYPIPNTYVVDRPSAEGDRHGQSSLDCGPCPVRRSGALHQSSQAAHRRSMTGAKQKTPIRVVRFSIDHSALTPAQVVAFARHAGAARQTWNWALGRWMDWRNNTKFYVDYKVFKAAGMGPGLSTDDLIQVIERAVSIRQDDKWMDAAWDEARQIHGEWDQFQKASTLQSLYLAGAQEPFDPSRDDGINPYHWWVTEGDKSGLPKAERHNVNSGATYTAPLRAFEEAVGRFYKLPGKKGTPKFKSKHDDEQGFCIQRLTETGLSPWRAIEGGHRIKVPSIGSIRVVQSTKRLRQLIKRGGKTTSARFTRRGGKWFVSVSVAFDLSAPRVQRPARLSRRQRAGGSTGVDLGVNRLATLSSGDQFPNRRLLRKSMAEIKRLQRKFDRQHRAGSPECFNEDGTHKKRCRWGREDGPAMSRSAQTTKRQLRRIHDLTARRRAGVLHEITKDLATRFELIGVEDLNVAGMTAKSKPKPDPDRPGHFLPNRRAAKAGLNRAILDVGFYEFKRQLGYKTEWYGSTMQMVHRYAATSKTCSGCGWVKPKLTLAERTFNCTQCGLAMDRDHNAAVNIRALALEGAAPMEREQPAPVGAAEKRHRDPVSHRRRPKSLAPCESTRPVRDLSPPATQEETA

SEQ ID NO: 54 - protein, 606 aa, unidentified
  source 1..606: note = Description of Unknown: cas12f sequence
MAQAEAPRRLRAYKFALDPTEAQLREFEQHAGSARWAYNHANAILSRYSDTLRNRWNAWIAQHHGLSREQLYALPDRERTAIQAAARAAVKAENAQLAAELRIIDDHRKRVTHKGKPSVEPGEQPAEDAPERAYQLWRERVELARLHAEDPQAYRAERKRILDEIRPLVNATKRKLIEQGAYRPTAMDISTLWREIRDLPPDEGGSPWWPEVSIYAFTSGFAHAETAWKNYLESLAGRRAGRPVGKPRFKKKRRSRRSFTLYGSVKLVTYRRIQVPSIGSVRLHGSAKRLHRALERRGGIIKSITISQGGHRWYASVLVDELDITPGRETQRGPSRRQRDRGAVGVDLGVHHLVALSDPNEKTLDNPRHLRKARKRLLKAQRAMSRRRGPDKRTGQEPSRRWVKARNRVARLHHELAVRRAGHLHEITKRLATSYELVAIEDLNVAGMTRSARGTIDQPGRGVRAKAGLNRSILDTSPAEFRRQLQYKASWYGATVAVIDRWAPTSRTCSSCGAVKAKLSLAERTFFCEHCGMELDRDINAARNILAFAQSAYPGEGKALNACGGSVSPGSQSVVQAGADEAGRPARKPRRSSRGSDPPATPTTRA

SEQ ID NO: 55 - protein, 1421 aa, unidentified
  source 1..1421: note = Description of Unknown: cas12a sequence
MTSSSPTQRAYTLRLKSAAQGDKSWAEKLWDTHEIVNKGARAFGDWLLTLRGGISHKLENLNDKETGEEGKKRRRILLALSWLSVESKDFAPEKYIVEKDGEDKHRTKEALEAILKSRNLEDEEVESWVNDCKDSLTSSIRDDAVWVNRSRAFDDAVRKIGDSLTREEIWDVLGRFFGKKEAYLAPRTIDEKNGKTKKEEPKDLARKAGGWLSKRFGKGKGTDFSKLSKVYSEIVKWAEEPRKSEPRTLANLASALKEDSLQGILNLIKNSGSKSGTRNFLEEIGEGEVSKENLAILKAKAEGNRNYCKKEIGGKGRREWSDRILKSIEETLDGKFTYLQEKGPARHWEFAVMLDHAARRISAGHTWIKLAEARRRNFEEDSQKINEVPENARQWLETYREDRSKSSGAIEGYLISKRAVTEWETVVKAWKNCKTEEDRIAAAGALQDNLGIDQFGDINLFRALASEDVRCVWQVDGKPDANILLNYVAATKAEFDKRRFKVPAYRHPDPLLHPVFCDYGNSRWEIRFDVHEVNRTGKKAKQNKKTIETADVHGLKMDLWTGSKIENVSLRWQSKLLEKDLAVKQLDGKEDGKKEVSRASRLGRAAVGAGWETPVSASSVFAQKHWNGRLQASRKELSRIARRVKTRGWDEKANSMKKNLKWFITFSPKLKLQGPWISYVDNSEDKRPFTFTSKGEPILDEVFSIENKNRKGRARLILSRLPGLRVLSMDLGHRHAAACAVWETLSSRQLEDACAEGGYDKPAPDAMYHHIKSNRGKRVIYRRIGADELSDDSIHPTPWARLERQFLIKLQGEERKARMATADEIWEVHELERALGRKTPLVDRLTKSGWGSDSGTPRQRQLLGELNQWGWEPDEAQENSEDDEITSRESLLVDKLMSRTVDTVRKGLRRHGNRARIANFLVAREKTVPGGQMDTLNNEGRKEIIADALAFWYELANGGEWKDTEALDWWKIHIEPELSVEELPDIAGTGIAPKERKRKKKELKEKLKPVAERLLTSGAKKLSDQWCERWKQDDKEWQKTLRWLRDWILPRGVRGKSELIRNVGGLSLDRLTTIQSLYQAQKAYFTRITPKGIQMDKDKPLTAVMNFGGHILNDLENMREQRVKQLASRIVEAALGVGRVKIPKKSKDPKRHYERVDAPCHAVVIENLTNYRPEETRTRRENRQLMTWCSGKVKKYLSESCSLHGLFLWEVPPSYTSRQDSRTGSPGIRCEEVSVEKFFKTPFRQREVARAEEKDSKNKASAYEQYLIDLKERWKSRGEETALLRIPRKGGEIFVSANSNSPASKGLQADLNAAANIGLKAITDPDWSGSWWYVPCSSKDFVPIKDKIGGSRAFENITTPMPNPDDAKEATGKKRSGKKEIINLWRNPACSPLERDEWERTAKYWNMVEYHVIKRLKRQMG

SEQ ID NO: 56 - protein, 1333 aa, unidentified
  source 1..1333: note = Description of Unknown: cas12a sequence
MKNFQDFTNLYELSKTLRFELKPIWGTKKLIEEKNILKLDKKKRENYEKVKPYFNKIHQEFINFALRNPNFDFSQFEEKYLNWLKDKKNKDLLKEKESIDKIFLEKIWKLFENSVKDFLKENGFESIVKSEDQNLKFFRRKEIFEVLQEKYGSELETQMVNKDWEIKSIFNGWEKWLWYFDKFFNTRDNFYKTDWTSTAIATRIIKDNLKIFLENTIIFEKVKNKKIDFSEVEKNFSVSIDTFFEINNFNNCFLQDWIDFYNKVIWGETLENWEKLKWLNEIINKYRQDTGEKIPYFKKLQKQILSEKDWVFIDKIEDDGGFYEVLKNFYKNAAEKEWFLKNIFENFYTISDKNLEKIYFNKIAFNTISHKFWSALEFERILYEEMKKEKADWIKFEKKENKYKFPDFIQIIFIKRSLENYDSENLFWKERYYKSEENVDWFLEKNNNNIWEQFCKILNFEFLNILKRRIIDEAWEEYEVWFEISKNILWEKLENFELNQENKWIIKDFADYSLALYSFWKYFAVEKWRNWDLNIDISDDFYGWEDWYIEKFYNTGYDEIVKPYNLMRNYISKKPWEDSKKWKINFETSSLLSWWDKNLESNWSYIFQKWNKYYIWIINWSKPAKEVLEKLYSWNGEKIKRFIYDFQKPDNKNTPRMFIRSKKDSFSPAVGKYNLPVEDILEIYDNWLFKTENKDNSNYKESLSKLIDYFKLGFSKHESFKHFNFVWKDSKEYENIADFYRDVEKSCYQITSEFLDFEELKKLTFKKHLYLFQIYNKDFELDESLQKNWYNFRDEWQKNIHTKYFEALFLEENILRKSWAVFKLSWGWEVFFRKESIKAEKEKRKNIEVTKNRRYTEEKYFLHFPIQVNFKNEISWNFNQEINKFLANNPDINVIWIDRWEKHLAYFSVINQKWEILESWSFNKIENYNKNWEKLLFPEREIKEIHKDWSLIDLELVETWRKVDYVDYKLLLEYKERKRLLQRQSWKEVEQIKDLKKWYISALVRKIADLIIKHNAIVIFEDLNFRFKQIRGWIEKSIYQQLEKALIDKLNFLVNKNEINLEKAGSILKAYQLTVPVDSLKEIWKQTWVIFYTEAAYTSKIDPIKWWRPNLYLKKQNAEINKENILKFDNIIFNSKENRFEFTYDLKKFFWKDSKFPAKTVNTVCSCVERFKWNRNLNNNKWGYIHYENLTDWKLANKEQKEDEFSNFKELFEKYFIDINWNILEQIKNLDTKNNEKFFSSFIDLFTLVCQIRNTNQNAKWDENDFILSPVEPFFDSRKSQNFWKSLPKNWDENWAFNIARKGLIILNRISENPEKPDLLIFNADWDNFARNI

SEQ ID NO: 57 - protein, 1175 aa, unidentified
  source 1..1175: note = Description of Unknown: cas13b sequence
MTEQNEKPYNGTYYTLEDKHFWAAFFNLARHNAYITLAHIDRQLAYSKADITNDEDILFFKGQWKNLDNDLERKARLRSLILKHFSFLEGAAYGKKLFESQSSGNKSSKKKELTKKEKEELQANALSLDNLKSILFDFLQKLKDFRNYYSHYRHPESSELPLFDGNMLQRLYNVFDVSVQRVKRDHEHNDKVDPHRHFNHLVRKGKKDKYGNNDNPFFKHHFVDREEKVTEAGLLFFVSLFLEKRDAIWMQKKIRGFKGGTEAYQQMTNEVFCRSRISLPKLKLESLRTDDWMLLDMLNELVRCPKSLYDRLREEDRARFRVPVDILSDEDDTDGTEEDPFKNTLVRHQDRFPYFALRYFDLKKVFTSLRFHIDLGTYHFAIYKKNIGEQPEDRHLTRNLYGFGRIQDFAEEHRPEEWKRLVRDLDYFETGDKPYITQTTPHYHIEKGKIGLRFVPEGQLLWPSPEVGATRTGRSKYAQDKRFTAEAFLSVHELMPMMFYYFLLREKYSEEASAEKVQGRIKRVIEDVYAVYDAFARDEINTRDELDACLADKGIRRGHLPRQMIAILSQEHKDMEEKVRKKLQEMIADTDHRLDMLDRQTDRKIRIGRKNAGLPKSGVIADWLVRDMMRFQPVAKDTSGKPLNNSKANSTEYRMLQRALALFGGEKERLTPYFRQMNLTGGNNPHPFLHETRWESHTNILSFYRSYLKARKAFLQSIGRSDREENHRFLLLKEPKTDRQTLVAGWKSEFHLPRGIFTEAVRDCLIEMGYDEVGSYKEVGFMAKAVPLYFERACKDRVQPFYDYPFNVGNSLKPKKGRFLSKEKRAEEWESGKERFRDLEAWSHSAARRIEDAFVGIEYASWENKKKIEQLLQDLSLWETFESKLKVKADKINIAKLKKEILEAKEHPYHDFKSWQKFERELRLVKNQDIITWMMCRDLMEENKVEGLDTGTLYLKDIRTDVQEQGSLNVLNHVKPMRLPVVVYRADSRGHVHKEEAPLATVYIEERDTKLLKQGNFKSFVKDRRLNGLFSFVDTGALAMEQYPISKLRVEYELAKYQTARVCAFEQTLELEESLLTRYPHLPDESFREMLESWSDPLLDKWPDLQREVRLLIAVRNAFSHNQYPMYDETIFSSIRKYDPSSLDAIEERMGLNIAHRLSEEVKLAKEMVERIIQA

SEQ ID NO: 58 - protein, 1115 aa, unidentified
  source 1..1115: note = Description of Unknown: cas13b sequence
MESIKNSQKSTGKTLQKDPPYFGLYLNMALLNVRKVENHIRKWLGDVALLPEKSGFHSLLTTDNLSSAKWTRFYYKSRKFLPFLEMFDSDKKSYENRRETTECLDTIDRQKISSLLKEVYGKLQDIRNAFSHYHIDDQSVKHTALIISSEMHRFIENAYSFALQKTRARFTGVFVETDFLQAEEKGDNKKFFAIGGNEGIKLKDNALIFLICLFLDREEAFKFLSRATGFKSTKEKGFLAVRETFCALCCRQPHERLLSVNPREALLMDMLNELNRCPDILFEMLDEKDQKSFLPLLGEEEQAHILENSLNDELCEAIDDPFEMIASLSKRVRYKNRFPYLMLRYIEEKNLLPFIRFRIDLGCLELASYPKKMGEENNYERSVTDHAMAFGRLTDFHNEDAVLQQITKGITDEVRFSLYAPRYAIYNNKIGFVRTGGSDKISFPTLKKKGGEGHCVAYTLQNTKSFGFISIYDLRKILLLSFLDKDKAKNIVSGLLEQCEKHWKDLSENLFDAIRTELQKEFPVPLIRYTLPRSKGGKLVSSKLADKQEKYESEFERRKEKLTEILSEKDFDLSQIPRRMIDEWLNVLPTSREKKLKGYVETLKLDCRERLRVFEKREKGEHPVPPRIGEMATDLAKDIIRMVIDQGVKQRITSAYYSEIQRCLAQYAGDDNRRHLDSIIRELRLKDTKNGHPFLGKVLRPGLGHTEKLYQRYFEEKKEWLEATFYPAASPKRVPRFVNPPTGKQKELPLIIRNLMKERPEWRDWKQRKNSHPIDLPSQLFENEICRLLKDKIGKEPSGKLKWNEMFKLYWDKEFPNGMQRFYRCKRRVEVFDKVVEYEYSEEGGNYKKYYEALIDEVVRQKISSSKEKSKLQVEDLTLSVRRVFKRAINEKEYQLRLLCEDDRLLFMAVRDLYDWKEAQLDLDKIDNMLGEPVSVSQVIQLEGGQPDAVIKAECKLKDVSKLMRYCYDGRVKGLMPYFANHEATQEQVEMELRHYEDHRRRVFNWVFALEKSVLKNEKLRRFYEESQGGCEHRRCIDALRKASLVSEEEYEFLVHIRNKSAHNQFPDLEIGKLPPNVTSGFCECIWSKYKAIICRIIPFIDPERRFFGKLLEQK

SEQ ID NO: 59 - protein, 1115 aa, unidentified
  source 1..1115: note = Description of Unknown: cas13b sequence
MESIKNSQKSTGKTLQKDPPYFGLYLNMALLNVRKVENHIRKWLGDVALLPEKSGFHSLLTTDNLSSAKWTRFYYKSRKFLPFLEMFDSDKKSYENRRETAECLDTIDRQKISSLLKEVYGKLQDIRNAFSHYHIDDQSVKHTALIISSEMHRFIENAYSFALQKTRARFTGVFVETDFLQAEEKGDNKKFFAIGGNEGIKLKDNALIFLICLFLDREEAFKFLSRATGFKSTKEKGFLAVRETFCALCCRQPHERLLSVNPREALLMDMLNELNRCPDILFEMLDEKDQKSFLPLLGEEEQAHILENSLNDELCEAIDDPFEMIASLSKRVRYKNRFPYLMLRYIEEKNLLPFIRFRIDLGCLELASYPKKMGEENNYERSVTDHAMAFGRLTDFHNEDAVLQQITKGITDEVRFSLYAPRYAIYNNKIGFVRTSGSDKISFPTLKKKGGEGHCVAYTLQNTKSFGFISIYDLRKILLLSFLDKDKAKNIVSGLLEQCEKHWKDLSENLFDAIRTELQKEFPVPLIRYTLPRSKGGKLVSSKLADKQEKYESEFERRKEKLTEILSEKDFDLSQIPRRMIDEWLNVLPTSREKKLKGYVETLKLDCRERLRVFEKREKGEHPLPPRIGEMATDLAKDIIRMVIDQGVKQRITSAYYSEIQRCLAQYAGDDNRRHLDSIIRELRLKDTKNGHPFLGKVLRPGLGHTEKLYQRYFEEKKEWLEATFYPAASPKRVPRFVNPPTGKQKELPLIIRNLMKERPEWRDWKQRKNSHPIDLPSQLFENEICRLLKDKIGKEPSGKLKWNEMFKLYWDKEFPNGMQRFYRCKRRVEVFDKVVEYEYSEEGGNYKKYYEALIDEVVRQKISSSKEKSKLQVEDLTLSVRRVFKRAINEKEYQLRLLCEDDRLLFMAVRDLYDWKEAQLDLDKIDNMLGEPVSVSQVIQLEGGQPDAVIKAECKLKDVSKLMRYCYDGRVKGLMPYFANHEATQEQVEMELRHYEDHRRRVFNWVFALEKSVLKNEKLRRFYEESQGGCEHRRCIDALRKASLVSEEEYEFLVHIRNKSAHNQFPDLEIGKLPPNVTSGFCECIWSKYKAIICRIIPFIDPERRFFGKLLEQK

SEQ ID NO: 60 - protein, 1008 aa, unidentified
  source 1..1008: note = Description of Unknown: cas13b sequence
MDTPNFSERIPVSLQSHPYYFAHYLNMARHNAYVILEYVNRELIKPGKNLDEDNLIQSTVLKDGYFDRKPDELSHRNRLLVQHFPFLREAENEGARTCNPVSYKLKTALAALNQWRNNASHYPLNQNHEKDFDLQPFFSFAIEACKKRMREVFQPDDFYLLETNEKQFYTLHNENGFTEKGLYCFICFFLEKKYAFQFLAGIKGFKNTTDNKFRATLETFTEHCCRLPKPKLDSSDIKLDMLGELSRCPAPLFDLLDIEERKKFIREPEEVKPDESGDREEVQQVLMKRYDDRFPYFALRYFEEKNLLKGISFHIHIGRWIKSEHTKKIMGAERDRRLLKDIRTFGELKEFSPEHAPDYWLRDGITPDDVDQFSPQYRIVGNRIGIKLNYNGHNRWSVPDKEINVKPDAIISTYEFLNLFLYEHLYQKKLTGLSPAEFIQDYLDRFNNFLSEFKAGHIRPVGDFSLEKRRGQGDEPDLTARRKSLQKELDRFVLKGKDLPDKIREYLLGYKQKSEKKQAKWILGGMIKETVYWRNKAEQSPEKMRSGDMAQQLARDIIFLTPPHTVKEHKQKLNSLEYDVLQYALAYFSSNREKLYSFFKEHQLTVKGDRAHPFLYKIRLDECQGILDFFIVYMQQKEKWLGWLDRNLKSPRLNEEEFFNTYSYFIKTDTKRAIEMDYESCPNYLPRGIFNEPIAKALQKAGVKIKDEDNASYALSVYSNGKTQPFYNKERYYNKGIFRMEELPEKLQPKELLGKIQWTIKSSGKDTEEFRSLQNLKNRILNTEKEIRYVQSTDRALWIMVADLFPETFELRPDDLECIGHDLSDDLLSRPYQMKEKVYNYTITDYLPIKRYGEFRRFLKDRRLENLLTYFEEGVPLHREALVAELEAYDLQRKNLLEIIYRFEKLVFDRHRHELTFSGEGENQYVNHWDYLDFVARKYGLSAEVKELNSERFTELRNKMLHNQIPYQLWIKEAIAAREENTVCGRIMGMIGEIYERMTTEIEKQMQV

SEQ ID NO: 61 - protein, 1063 aa, unidentified
  source 1..1063: note = Description of Unknown: cas13b sequence
MFDNEQKNLEKEPYWGVFLNQARLNAYIALRDISERLEENAADEDSLSEWPVLKYLDNDTDAVKSRRIFDLVEKHFSMLKIIYGGEKEGDLVKRSKEYKIILKCLFRALNFYRNKFCHMYSGNRARKYNEKELIKYLEDCFDASVRKIKELRRLDEKDVLHLRRKIAEGKDANKRVIDNPQFRYPFKNEKGELNEKGLYFLASIFLDKKEAHEFLKKQEYFKNDSEPKYRATLESFYHYRIKLPRPVIESDVDKNGLALDMLNELKKCPKELFDLLSKEQQEKFRVVDSEDADEEGNEILMRRYSDRFPYLALRYCDENQVFERIRFQIDLGRYYFKFYPKETIDGKTQQRSLDKRLKIFGRIKDVKSKVEQEWSGIIKSPDTIEENPNEPYKLKTTPRYNIVDNQIGFVITGDKNLPDVKRPDGRIELEKPDGWLSIYELPGMLFHGLKYGFDKTERMIKIYIEKQRKICKEICEKGTITPDDGESMPEALKGGAKAAKRNYSEKKLERMLQDTEQRIRAIQTTQKRMDEPGNKPGKKKFFDIRAGKLADFLARDIMALQRFDPAKHGKDKLTAINFQVLQATLAFYGAKKDVIEDMFKGIGLLEGDNPHPFLNQIDPAQYNSIAGFYQAYLQKKRSYLEDYRKEEEYDEQFLRPKRQRYAQEKREIKTVARQLLDNPVNVPKNFFKKEIEEFVFSQDPSLKKSKMNTAYMIQALFEKHYGRQQPFYSYNRTYPVVSKAIEYGKKGKNKKIAKVLMAIEPKLNYMEIKKIVNEMPDGQYEPENLKRNLYEGYKDYEKDERIIRRCKVQDVVSFMMVEETLKDQLDFNGNVLTLEKITPWEASPFKKPVLCHTIISIPFNTKGGHTDKDYVDFIKNNFEGSYDCEPNKIILKYKVTSKDTKLKDIGKYRMYSHDRRLPGLLIWKYRPNDQNGNEIKFTEIEQEIKAFERRRIEIAQCLYTLEKKVIDSWFTQDELGEEHIPFNKVIDVIKAKMPNFEDKCNVLLKIRNAINHNQFPVYEQAIQTAPGKEIAGKMLRITESYIEQIMAKIDPDFGRTEDAESSR

SEQ ID NO: 62 - protein, 1009 aa, unidentified
  source 1..1009: note = Description of Unknown: cas13b sequence
  VARIANT 375: note = MOD_RES - Any amino acid
MDTPNFSERIPVSLQSHPYYFAHYLNMARHNAYVILEYVNRELIKPGKNLDEDNLIQSTVLKDGYFDRKPDELSHRNRLLVQHFPFLREAENEGARTCNPVSYKLKTALAALNQWRNNASHYPLNQNHEKDFDLQPFFSFAIEACKKRMREVFQPDDFYLLETNEKQFYTLHNENGFTEKGLYCFICFFLEKKYAFQFLAGIKGFKNTTDNKFRATLETFTEHCCRLPKPKLDSSDIKLDMLGELSRCPAPLFDLLDIEERKKFIREPEEVKPDESGDREEVQQVLMKRYDDRFPYFALRYFEEKNLLKGISFHIHIGRWIKSEHTKKIMGAERDRRLLKDIRTFGELKEFSPEHAPDYWLRDGITPDDVDQFSXPQYRIVGNRIGIKLNYNGHNRWSVPDKEINVKPDAIISTYEFLNLFLYEHLYQKKLTGLSPAEFIQDYLDRFNNFLSEFKAGHIRPVGDFSLEKRRGQGDEPDLTARRKSLQKELDRFVLKGKDLPDKIREYLLGYKQKSEKKQAKWILGGMIKETVYWRNKAEQSPEKMRSGDMAQQLARDIIFLTPPHTVKEHKQKLNSLEYDVLQYALAYFSSNREKLYSFFKEHQLTVKGDRAHPFLYKIRLDECQGILDFFIVYMQQKEKWLGWLDRNLKSPRLNEEEFFNTYSYFIKTDTKRAIEMDYESCPNYLPRGIFNEPIAKAVQKAGVKIKDEDNASYALSVYSNGKTQPFYNKERYYNKGIFRMEELPEKLQPKELLGKIQWTIKSSGKDTEEFRSLQNLKNRILNTEKEIRYVQSTDRALWIMVADLFPETFELRPDDLECIGHDLSDDLLSRPYQMKEKVYNYTITDYLPIKRYGEFRRFLKDRRLENLLTYFEEGVPLHREALVAELEAYDLQRKNLLEIIYRFEKLVFDRHRHELTFSGEGENQYVNHWDYLDFVARKYGLSAEVKELNSERFTELRNKMLHNQIPYQLWIKEAIAAREENTVCGRIMGMIGEIYERMTTEIEKQMQV

SEQ ID NO: 63 - protein, 1160 aa, unidentified
  source 1..1160: note = Description of Unknown: cas13b sequence
MKTLGALSSHNYNNKKYYFSGLLNTAQYNFNLALQEVNDRLGKKGKNPGKTMIKNIFDQKDSFSTQERAMYYLEEFFPWIFLVMKQSGINIPTEEQETKLHKEEIQLIQEHLISLYELLDDLRNEQTHYMHDPVIIPEEVSKMLDALLLQILKNTRKKCKDDEYRTFIVKKYQEEFQKEIKVQVKDRFGKEKEKIVTGEVKENYVINRCFRKWIQKEGEEETLRYSTVQEEQGKYVWSSSGFVFFLSLFLRRKELEDVMNHVPYFKDSRKLLFYLTRKTFSSYCFRDLRKSLRSDYSNDSLLMQMIEELYKCPGELYEVLLKEQKQEFIEDINEYYKDNPEFEGSANEAQVIHPVIRKRYQDKFPYFALRFIDEYFNFPTLRFQLVLGEYVTDRRTKELQGTALFTDRVISQRISYVGKLSEAEMNKKREGYTETGWKEYPNPYYKIENNRIPLYIEFSKNEELIFKEKKFKYNTLAKWENREIDKRTGEFNQVNKQRRITQLEEFKIDNPKKMKTPNVFLSIYELPALLHALLIEKKTEAEIEDIIKAKIKKQLTEIAEGRRNLSGLPKGIKKMRNCNSDFEKKKLISDIDNEIKKGEKILEEVQQWLNPVINKKGTGKQENNKPFFSNTYRGKYATWLAYDIKRFTGKDHIQNWKGYQFSELQTLLSLYTLRKEELKNFLEKDLQLTSHPFLKEALKAVNLEDFMGAYLRGRQFFLEKAKKQIGIKGVKKSIFQYFEERKYKIYSSNLDYWEELWKHPVNLDRGLFDERGTVYNKNKELNDLQNRAAWFSFAETNPKQQFYHFPRIYSDEDITKPVTDRYGKTKEKLILFKLSPQKGFMEQIPSDLKKKYQEDKGKVEHPEVQKEKKYEEKKHPGINAFIKNAYKNEQKIRRISRNDIFLYEMVKYMLNKISPATEFSSLDKVWLTRIEREKQATEAREQSFKEKGDTSENKIRQDYLLSFPITLTLFNDIIKEKVKIKDIGRFRKLEKDERVQTMISYYTSGLWKNDQPSLTIKELEAELESYHKIRLQEIFKEVHKLEKEIYEFTPEEDKSKLLARESFPKFKYYISFYFIPKEDQEVFNEIQFDKYKNLEQIPGRKPEYDPYYLLIFIRNKFAHNQLPAEPIYKTALTFLPNNFNTLAEYYHKLFILLNNKNYNN

SEQ ID NO: 64 - protein, 1160 aa, unidentified
  source 1..1160: note = Description of Unknown: cas13b sequence
MNILPAAPEKEKIAYSTATAPWFFGAFLNQARHNLFLTVNDLAIRLGEKVIDYDDQLLNSNVVRMLVNEKASPLQLEILMKYLDRHLPFLIPMQVALKGHQGDASDNPVIGSPADYGAILSKLIVCLNAARNHFSHYHSTSGWSGYNEVIEWMEHVFTRNIETVVKRFTLTEEEVQHLKKPVDKSPKGTIPPYYFSFCKGDIWTDTGLAFFICLFLTREEAYLFLKKLRGFKRGEERFHKATLEAFCVGSLKVPRERLESNNSPQSAFLDMCNELVRCPKSLFDLLEPEKQELFRRDPEPEDAEDNGIEEEEDQPQALLVRKENRFSYFALRYLDIAKAFPRLRFGVDLGTYFFSVYPKTFAGIEETRQLSKRLIGYGKLEEFAREKRPEHIAALFRSKEEANAAPTEPFIRETAPHYHLDGNNVYLYMSGDGEAQWPAVELEEVTGKSYPRKLVKKSTLLPFAVLTVNELPALLFYHLLHKEKGAGDAAERVIINHMERVKRFFKALQDDKVDQVAGQPIRKPDVDADESLHMEYDRRWKLLKKKLSEYQLRASYIPEKIINYLLNIEAVDLGDKAMAQLKNLQRQAQDDIAAIERRMEHLMKKGADGRKTLKVGNLAQQLAEDMLQMQPVQIGTDGEPVPASKANNLAFRLLQSHLAYFAENRHNLPAVFEACGLIGASNKHPFLDNINIESCKGVVDFFILNFRNKLDFLDRCLQEGEWHRYHFISAAKLKSGAKVTIKKYLNEAFESKGRNHIPFTLPPSLFLDASLDWLAKFGDGKAKKVLAENEYVNSVFLIRRLFADGGLQPFYAWKREYRLFEKKAGKAVFLDEAGRMRKADKIGIEVERHREFLARPVKKGKQYDIKKAAAEQFLRSYRFYLQEEKYIRLLAAQDMLLFRCICDLLTYHVGDIGLEELAEAKAGTFSLANITPEKTETAKSLLNYRPAGGVVLDRHFYATDEKGAFVKQEGKLVPGGQVRIFDNTLKIKNAGNFRKLLKDRRMNNLFFYFKQHADEPVVLHRMVLENELRAYDRMRLKVLPVIAEFEKKLYQHCTDVEKERLVVNGSMHHRCYLDVYREKYQPDWGWEAAGNLLRIRNAFVHNQFPLMEGDGFKLEVAHWKKINADFVPSEQGSSLGYGIIDRLGQLAVEGYEGLIKNIHV

SEQ ID NO: 65 - protein, 817 aa, unidentified
  source 1..817: note = Description of Unknown: cas13b sequence
MGEEEQAHILENSLNDELCEAIDDPFEMIASLSKRARYKDRFPYLMLRYIEEKNLLPFIRFRIDLGCLELASYPKKMGEENNYERSVTDHAMAFGRLTDFHNEDEVLQQITKGITDEVRFSLYAPRYAIYNNKIGFVWTSRSKKKSFPTLKKKEGEGHRVAYTLQNEESFGFISIYDLRKILLLSFLDEGKNIVSGLFKQSKANWENLSENLFDAIRTELQKEFPVPLIRYTLPRSKGGKFVDPKLADKQEKYESEFERRKEKLSEILSEKGFDLSQIPRRMIDEWLNVLPTSKEKKLKGYVETLKLDCRERLRVFEKREKGEHPVPPRIGEMATDLAKDIIRMVIDQGMKQRITSAYYSEIQRCLAQYAGDDNRRHLDSIIRELGLKDRKKGHPFLGKVLRPDLDHTEKLYQRYFKEKKEWLEATFYPAANPKRVPRFVNPPAEKQKELPLIIHNLMKERPEWRDWKQRKNSHPIDLPSQLFENEICRLLKDKIGKESSGKLKWNEMFKLYWDKEFPNGMQRFYRCKRRVEVFDKVVEYEYSEEGGNYKKYYEALINEVVRQKISSSKENSKLQVEDLTLSVRRAFKRAINEKEYQLRLVCEDDRLLFMAVRDLYDWKEVQLDLNKIDNMLGEPVSVSQVIQLENGQPDAVIKAECKLKDVSKLMRYCYDGRVKGLMPYFANHEATQEQVEVELRHYEDHRRRVFDWVFALEKSVLKNEKLRRLYEKSQEGCEHRRCIDALRKATLVSEEEYKFLVHIRNKSAHNQFPDLEFGKLTPNVTSGFCECIWSKYKAIICRIIPFIDPERRFFGKLLEQK

SEQ ID NO: 66 - protein, 1114 aa, unidentified
  source 1..1114: note = Description of Unknown: cas13b sequence
MRIPKLIEEHKSVFGAYSTMALSNVETVLNHIAERAGLDGYERDRGPGVEDYWEHPVMQCLCRKDKPRSIPSDVLLDVRNRLFRSFPFLKIMAENQRDYRNAKGKVECVEINESDIFVVLNNSFRVLKAYRDTCTHYLIENRIWEDNSPMLMYNECPLAAMVNQYYTAALRVTKERYGYETRDLTFIQKRRFKQEPEKEASGNVKKKAVPDLAFFLSLVALNGDGRKWLHLSGWGVVLLICLFLEKKYVNVFLSKLPNPGNYPPSSKERRIIRRSMGVCSVVLPKERIHSETGDLSVALDMLNELKRCPRELFDTLSPGDQERFRTISSDHNEVLQMRSKDRFAQLVLQYIDHNRLFENLRFHVNMGKLRYLFNPKKYCIDGQTRVRVLEHPLNGFGRLQEMEEKRLQENGPFARSGIKVRCFDEVRRDDANESNYPYIVDTYTHYVLENDMVEMFFCPEGSGMKMPEVTSREGKWYVDKKVPHCRMRMSVLELPAMLFHLLLCGAKNTEVHIGKVCDNYCHLFSDMAQGNLTEENILSYGIKKEDIPQKVWDCVRGVHMGKDSRAYREKEIRERYEDVTRRLERLEADRKAVLGGENKIGKRGFVQIVPGRLAAYLATDICRLQPSLRKGDGYGTDRLTGLNFRLLQSSIATYNCGESDILYGRFRDVFCSAGLIGGDNPHPFLDKVLPEAYSVCCPRNTIEFYERYLEEYQRYLKPLVIKLEKGKVPSLSFVNEGQRRWARRDDAYYHELGNLYLSQAIELPRQMFDDEIKDKLREMPEMRDVDFDHANVTFLIGEYLKRVRHDESQEFYSWPRHYKYVDMLKCILNPKNGSLQAVYIQMGEREGLWQERSELEEKYAKIRLRDLGRKGLDKDEANERIKTGLGNRKKEYQKAEKVIRRYKVQDALLFMLAKNTLFNSVEVDDERFKLKDIMPDGEKGILSEVVPMDFCFRSGNSATRKLMGTIHSDNTKIKNYGDFFALANDKRMVTLLPLVGEQCLVKEEVKEEFDKYDDCRPEMISMVFDFEQWAYSAYPELKELVSNEAIKGRLFSNLLQELLGRGELTYEEKYALVGIRNAFLHNSYPKDGGVVKVRTLPDIAKSLKDVFKEYIRLE

SEQ ID NO: 67 - protein, 909 aa, unidentified
  source 1..909: note = Description of Unknown: cas13b sequence
MKMFYKSVLTAFFTAVDSLRNKYTHYSHKDLNIREIKIECTLGGKDYCIGLLNALDCIYDSAVNLLKLRFMAGEDEVAHLRRCKAVNKKVVVRTEKDGFYYRLSDNGGVTEKGVIFIASMFLNRKYGFLFLKQLEGFKRSDEKRYRLTLEAFLAFSNIKPVDRLKSDKLDRASLGLDMLNELTKIPKELSETLSVDCLYKYLASDGEDDLRSRIRYQDRFVPLALEFISQSDEFKDFRFYTYVGNYVYKGYIKRLIDGTDKERYLSDRLCGFYKSVNDASSDAIAQKYGVEIKDSNEPDYMLPDSFRPHVLRATPHFVINTNNIGIKICGNDCLPIVNGKGVESPEPDYWLSIYELPAMLFYAYLREKNGKRFKDYKSIRELIEGVEKKADEKNDRDKGALMARHIDKEIIWTQTKLDEVKRLEEKKVAAYGKKGRVVLKAGRMADLLAHDMVRLQPATKGSDKITGANFQALQVSLAYFKRDILADVFSRAMLTTGNHRHPFLYRIDVSHCSSLRDFYVAYLGERRKYFEDVAKKIAKNKLNTPCHILRRLQREGSGEEAGKDVKPKFLPRGIFTDSIKNCLEQSKLNIYIRNARNDVKPAINAAYLILMYYKEIEKGEFQGFYGEKRRYDILEEGKPLDLAERKKALASIKPAKIDVSEANMPMSKEEHLMRKRYHAVCNNESAIRMYQVQDILLLLMAKDIFKKALSEGVMSKKIGLENLNGIFDAPVNFVKNFDNIKLTATGIKIKDYGKVCRLGTDFKFNSLIKAFHKVYSKSVEMDYSDYLKEEEEFEKYRLNMVKLCREVERGITEDLHLSLDGKSHLSFNDDVIKPYNDKYNVFNGGDLTFFINARNMFMHGDYKYECVKYVVSEHFKGSLNDVSFAKETYGHFCNLLESMRKKTGLRIDI

SEQ ID NO: 68 - protein, 821 aa, unidentified
  source 1..821: note = Description of Unknown: cas12g sequence
MLPTRYKPARTLVRPLGRLPHEPRKEFVEKCRRVRMHFEQFNIDVADLCQWLMSLRPNTRIGDAQSTVFWDFFLNPSILTVEADEKERDRWRLAAFDELLQIRFGHDPNAPPWSEEFRSAIRHVAQRPKSATAQRLFDRLRSLTAPHRLVLLKSAAEWIIARYQRGMENWQRQFAEWQREKEEWEAAHPNLTPEVRDAFTRVFKNLFENPDGDGKIGVRRKNPRICSWERLKLNKDNCVYAGQKGHGPLCWEFSKFVKAQKNAGTIKTFFVDVANKYLHVRRNLSKPGVKLKKSPRQEAFKRLYNQKGMEKARNWFTDAWSGYLTALNLNEKTILDHGCLKHCGAIGAEFEKSLCQFNPHTHLCVQYRNALESLEPAIRELEGDYREWRRLFLAPPRKPSFRYPSSRRLPMPKIFGEHFHQIDFDQSILRLRLEDMAEGEWIEFGFKPWPKDYRPGKDEVRVTSVHVNFHGNRMRAGFHFEAPAKPSRFACTQDELDDLRSKQFPRQSQDRQLLEVARRRLLESFDGMLESDLRILAVDLGEKGAAAAVYQGHGHEADVAIPIVKIDRLYDHVPDVLDVESARVPPPKFDDSRDPRGVRKEHVGRHLGQLQRGAQTLAQHRQQDESAPAALRRHDFRSLTRHIRWMIRDWTRHNAAQITAAAETHRCHLIVFESLRGFKPRGYDQMDFAQKARLAFFAYGRVRRKVVEKAVERGLRVVTVPYGFTSQICSECGHRQRNKGRLRKNKYQRRFVCECGEPKKSANKTAAPDRSATVSPCTCRLQLGSDVNAARVLARVFWDEIVLPTREEMREPAVDSAPPSK

SEQ ID NO: 69 - protein, 797 aa, unidentified
  source 1..797: note = Description of Unknown: cas12g sequence
MCLCTLSGRTRQEEEIIGSTQYTEARSLVRRIRRPRGESRRQFKSNVLLLRRHFEQFNVDASEICQWLMGIRPGGRHADESTGPFWEFFLDPGRFLRETGRGPEDADERIDAYRRIAFDVVAGIEDESRMSDPSIPRQIVESLHAVSMATRTESARRLFERLAGLEPSHRQILLKAAAEWIVSRYWRSVQGWPDRYKHWSDEKEEWEKAHPRLTESLREEFTGIFRDLGIRRKKPRVCPWERLEKGMDNCMYAGERIKVGYSRQSHSQLCAKYERFSYKQRQRTKSGKNFKSYFVKNAELYLKLRRKNRSLIKKDVMKLFRKKVPQALWFEKAWDEYLKALGVDEATLTKDGKLPHCTQFADDKECLFNRHTELCLQYRERLLRLPHLQELEQLYREWRDKYLSGPRRPSLRYPSKRTLPMPKVFGRGYFCADFTNSLLDLRLEGMGEGDFVRFGFAPWPADYDAQPSDATVTSVHIHFVGTRARAGFRFQAPHKTSRFASSQDEIDDLRSRKFPRAAQDGEFLDAARKLLLESFTGDAEREMKLLAVDLGDRGAGAAVFEGRCFKEAMPLKIIKTDTLIDKPPPVTKTPRKGKPGKRESKRARGLDKYHVARHLDTWRKGARKIAERRAKGEADPVKLGAHDMRSLSLHVRWMIRDWVRLNASQIIKTAESHKTDLIVLESLRGFSAPGYHKLDDEKKRTLAFFAYGRIRRKLTEKAVERGMRVVVAPYLRSSQVCAECGREQIDRNKLMKDKRKRRFICEYSDCTWQCDSDQNAACVLGRVFWGEIELPSERKKD

SEQ ID NO: 70 - protein, 830 aa, unidentified
  source 1..830: note = Description of Unknown: cas12g sequence
MHPSRYKTARTLVRRLCRLPGEDRSAFRSKVGLLRGHFEQFNVDVSELCQWLMSLRKRNKVPENPATFGALGDFLLQPGLPGEETDEKEADRLRLAVFDAVAGFRMLEDRLAASIPASLSDAIRDEAVFLAGVRAAGKPSGLARVLARLEACAPAQRLVLLKSAAEWIVARFLRGTENWMRQRAEWEKEKAAWEAAHPHLTPEVRAQFNKIFESLHDPENSGKPGVSRKNPRICPWDRLKQNLDNCCYGEKGHSALCWRYQDFLKQRMGENRRDKKNFSATAMDLAQICREWKIQHSRNALNNPRVLDRLFAEHERRKQDKTKKESRSPKPRQGGYKANPKADYLRSFKAHWKAYLEHMKLNDTTVLERGCLPHCLSIKKNGKESTCKWNKHTELCLEYKRSLAPLPDSVLELEPEYREWRRLYLHGPGRPHFRYPSAGELPLPKVFGEGFHQVDLDRSIVRLRLEGAAEGEWLEFGFIPWPRGYQPSRREVLITSVQVHFVGTRPRAGFRFDVSHRTSRFGCSQDELDELRSRRYPRQAQDKEFLAAARAQLIQTFEGGEGAARQQMRVMSVDLGEGGACASIYEGRTHQKDESLKVIKIDRRYDQHPEVLEKDVGAAKPQKFEKSDPRGVRKEHVARHLNRIAAGASAIAEHRRKERSDAECSVGELQEHDFRSLKRHIAWMIRDWVRLNAAQIIDVAKQHCCDLIVFESQRGFRLPGYDELDRGKKQRFAILAFGRIRRKVVEKAVEHGMRVVTVPYFASSQVCSACKRVQENRGSWRENKKKRVFACEFCKLKLNSDANASRVLARVFWGEIELPEPTRAHLPSKA

SEQ ID NO: 71 - protein, 864 aa, unidentified
  source 1..864: note = Description of Unknown: cas12g sequence
MPVSRYSESRTLVRPLARLPHEERQDVTPKVARLRRHFERFNVDVAELCQWLMGLRNQFGPKESPASFGPLGDFLIEPALDNIDADETERDRWRLAVFDAVAGFRPIRGLGDHPVPDTLRLAMQQAASLSPTPTTARLLERLRPLSPAHRLVLLKSAAEWIVARYQRGMENWVIQHAAWHKEKEAWEREHPALTPAVRERFTALYKQLSDSKPTDRPVSRRKNPRICEWERLRQNIDNCCYAGEKGHGPLCRKYANFVKARKAVDGKFNDLLFWDTATSFIALCRKFNVTRARNALQSQLDALFAEDQRRKAERDQAKGRQPRPLHPQAAARAKSDFLRIFKDGWNAYLSAMGLNDSTAIEKGRLPHCQKIGGTFENSKCEWNPHTDLCHQYRRLAGQLDDATLALEKDYREWRRLYLAGPRKPSFQYPSSRDLPMPKIFGAGFFELDMDRSILRLRLDDMVEGEWLEFGFKPWPREYTPSRAQVARPGRITSVHVNFIGSRCRVGFRFEAPHAGSRFGCSQDEIDQLRRDHPRERDDQPFLEAARKRLVETFAGDARRDLRLLAVDVGEKGCCAAVYQGTRYVADALLPIIKINQLYTEPPTELKPDSHNRPAPDRRPFNDEKDPRDPRGVRKEHVARHLKRMADKAPEVAAYRLAQREKAAPSPSASPPPVTLGVHDFRRLKRHVTWMIRDWARHNAARIVAEAQRHGCDLIVFESHRGRRPPGYHEVGDDAERRKLDNATFAFGRIRRKVTEKAVERGLRVVTVPYHCSSKVCSRCGRLQENDGLLRRNKKERKFICEQCKFETNSDGNAARVLARVFWGEIMLPSPEERRKKREGSGGRSPTPANPGGLVDAPPSRRNLR

SEQ ID NO: 72 - protein, 1263 aa, unidentified
  source 1..1263: note = Description of Unknown: Cas12 sequence
MEDYSGFVNIYSIQKTLRFELKPVGKTLEHIEKKGFLKKDKIRAEDYKAVKKIIDKYHRAYIEEVFDSVLHQKKKKDKTRFSTQFIKEIKEFSELYYKTEKNIPDKERLEALSEKLRKMLVGAFKGEFSEEVAEKYKNLFSKELIRNEIEKFCETDEERKQVSNFKSFTTYFTGFHSNRQNIYSDEKKSTAIGYRIIHQNLPKFLDNLKIIESIQRRFKDFPWSDLKKNLKKIDKNIKLTEYFSIDGFVNVLNQKGIDAYNTILGGKSEESGEKIQGLNEYINLYRQKNNIDRKNLPNVKILFKQILGDRETKSFIPEAFPDDQSVLNSITEFAKYLKLDKKKKSIIAELKKFLSSFNRYELDGIYLANDNSLASISTFLFDDWSFIKKSVSFKYDESVGDPKKKIKSPLKYEKEKEKWLKQKYYTISFLNDAIESYSKSQDEKRVKIRLEAYFAEFKSKDDAKKQFDLLERIEEAYAIVEPLLGAEYPRDRNLKADKKEVGKIKDFLDSIKSLQFFLKPLLSAEIFDEKDLGFYNQLEGYYEEIDSIGHLYNKVRNYLTGKIYSKEKFKLNFENSTLLKGWDENREVANLCVIFREDQKYYLGVMDKENNTILSDIPKVKPNELFYEKMVYKLIPTPHMQLPRIIFSSDNLSIYNPSKSILKIREAKSFKEGKNFKLKDCHKFIDFYKESISKNEDWSRFDFKFSKTSSYENISEFYREVERQGYNLDFKKVSKFYIDSLVEDGKLYLFQIYNKDFSIFSKGKPNLHTIYFRSLFSKENLKDVCLKLNGEAEMFFRKKSINYDEKKKREGHHPELFEKLKYPILKDKRYSEDKFQFHLPISLNFKSKERLNFNLKVNEFLKRNKDINIIGIDRGERNLLYLVMINQKGEILKQTLLDSMQSGKGRPEINYKEKLQEKEIERDKARKSWGTVENIKELKEGYLSIVIHQISKLMVENNAIVVLEDLNIGFKRGRQKVERQVYQKFEKMLIDKLNFLVFKENKPTEPGGVLKAYQLTDEFQSFEKLSKQTGFLFYVPSWNTSKIDPRTGFIDFLHPAYENIEKAKQWINKFDSIRFNSKMDWFEFTADTRKFSENLMLGKNRVWVICTTNVERYFTSKTANSSIQYNSIQITEKLKELFVDIPFSNGQDLKPEILRKNDAVFFKSLLFYIKTTLSLRQNNGKKGEEEKDFILSPVVDSKGRFFNSLEASDDEPKDADANGAYHIALKGLMNLLVLNETKEENLSRPKWKIKNKDWLEFVWERNR

SEQ ID NO: 73 - protein, 1222 aa, unidentified
  source 1..1222: note = Description of Unknown: Cas12 sequence
MKKFTNLYSLSKTLRFELIPQGKTLENIQKSGILEQDNSRAEKYEKIKKIIDDYHKFFIEKSFTGKKIDDYFLNQYFELFKIKDKDEEQKKDFKSIQENLRKNIISFFDKNKLKRLFEKEIIKEDLPNFVKEEEDKKLISEFDKFTTYFVGFHENRKSMYSEEEKSTSIAYRTINENLPKFINNIFVFEKISKTPISENFRELYKDLEEYLNVNDIQDIFKLNYFSNVITQKQIDVYNLVIGGKTLENGTKIKGLNEYINLYNQNQTDKKNKLPLLTVLFKQILCDRDTISFLPEQFENDIDVLDNIKNTYSNMEKSIKDIKDLLSNLKDFDLSKIYITNDIALTDISQQVFNNYSIIINAIKENIKKENPKKKTENEEKYGERIDKIFKSNNSFSIKYINDCIKEKNIEIYFMDFGKKENNKKVKNLFDELQNNYSMVKDLLEYKKIQSLIQDEKSIELIKNFLDSIKNIQHFLKPLYVKDNDIVKDISFYRDFEELYLNIDKITPLYNKVRNYVTQKPYSVKKIKLNFENSTLLAGWDLNKERDNTCAILRKDDLYYLAIMDVNNRNVFNEKGIDGIGYEKMEYKLLPGANKMLPKVFFSKSRIKDFNPSEQIIRNYEKETHKKGSNFSLKDCHKLIDFFKSSINKHEDWKNFNFKFSNTDKYEDLSGFYREVEQQGYKITFRNISKEYVDKLVEEGKIYLFQIYNKDFSKYSKGTPNMHTLYWKALFDEDNLKNVVYKLNGQAEIFYRKGSIEKENIVIHKANNAIENKNMDNKKKQSKFEYDIIKDRRYTVDKFQFHVPITLNFKAIGNERINEQVNQYIKDNNIKHIIGIDRGERHLLFLSLIDLKGNIIKQFSLNEIVNEYNGNSYKTNYHMLLEKREEERDKARKSWKTIENIKELKEGYISQVIHKITQLMIEYNAIVVLEDLNFGFMRGRQKVEKQVYQKFEKMLIDKLNYLVDKKKDKNEAGGLLKAHQLTNKFESFQKMGKQNGFLFYIPAWNTSKLDPITGFVNLFDTHYTNVDNAKKFFENFEDIRFNEKKNYFEFIVNDYTKFNTKAEGTKLNWTICSNEDRIKTFRSSSKNNQWVSETVNLTDSLIELFKKYDIDYKLELKEQIISKSEKNFFETLLYLFKLTLQMRNSITGTETDYLISPVADKTGNFFDSRKGIENLPNNADANGAYNIARKGLWVIEQIKKAKDLKKVKLAISNKEWLQFVQGK

SEQ ID NO: 74 - protein, 1262 aa, unidentified
  source 1..1262: note = Description of Unknown: Cas12 sequence
MAKNTIFSQFTGLYPVSKTLRFELKPMGKTLEKIKETGVIENDKKRHNDYFDAKKIIDKYHKYFIDAALSKFPRIDWSPLKEAIERSLDRSDASKKKLEKTQTEFRKKIAKALTTHDHYKELTASTPKDLFLKVFPDHFGKQPAIDTFDGFSSYFTGFQENRQNIYSDEAISTAIPYRLVHDNFPKFLSNIEVYKTLKDNAPSVLSDAENELRDFLNGKSLANIFELNAYNEVLTQSGIDFFNQVIGGISDEGGEKKTRGINEFSNLYRQQHPEFAQKRLATKMIPLYKQILSDRETKSFILESYSNDSQVQNSVKEFFESQILNWDIAGRRVNVLNELTSLVKRISEFDLGNIYVNQEELSNISLKLFDNWNSINGLLFKHAENRIGSAEKSANKKKIDAWMKNKEFSIATLNLAIAESNSEEISRVKIESYWNNFEAKVQSILCGDNRRNLDEFISATFNENNALREDSKIIEKLKAFLDALIEIMHSIKPLISDAENRDLSFYNELIPLYDQLSLVVPLYNKIRNYATQKLTESEKFKLNFDNPTLADGWDQNKEEANTAILLLKNGLYYLGIMNAKNKPKIKDFKTSESEDCYDKMVYKLLPGPNKMLPKVFFSEKGLATFKPPKDILDGYNAGKHKKGDLFDIGFCHQLIDFFKESIAKHPDWKKFDFNFSDTSSYEDISGFYKEVTDQGYKITFSKIPTSQIDEWVKEGKLFLFQIYNKDFAPGAKGSPNLHTLYWKSVFSPENLKDVVVKLNGEAELFYRPSSVKKPYSHKVGEKLVNRIGKDGLPLPESVFGELFRYFNGKLEGELSDEAKRYLDVAVVKDVKHEIVKDRRYTQDKFEFHVPLTLNFKADSKNEYMNERVRHFLKDNPDVNIIGIDRGERHLLYMTLINQKGEILKQKSFNVVESVNYQAKLVQREKERDAARRSWSSVGKIKDLKEGFLSQVIHEITTTMIENNAIVVLEDLNFGFKRGRFCVERQVYQKFEKMLIDKLNYLVFKNKPEGDVGGVLKGYQLAEKFDSFQKLGKQSGFLFYIPAAYTSKIDPTTGFANLFNMTELTSAEKKKEFLSHFEDITYDGKNDRFLFSFDYKNFKCFQTDYIKKWTVYSQGKRIVYDKESKSAKEISPVEIIKAALAKQNIALTDQLDVLSAINSVEASPKSASFFGDICYAFEKTLQMRNSIPNTDEDYLASPVMNKRGEFYDSRSCDDALPQNADANGAYHIALKGLYLIKNVFDAGGKELKISHEDWFKFAQSRNC

SEQ ID NO: 75 - protein, 1253 aa, unidentified
  source 1..1253: note = Description of Unknown: Cas12 sequence
MSKGKIWENFINQYSVSKTLRFELKPVGKTLENINAKGLIEEDEQRAEDYKKAKKIIDEYHKYFIEGALGSCSLDLNILNEFLQLYNKAQKTDADKKEYEKIQTTLRKNIAESFGKNADKKTKEQYENLFKKELLRNDLPDWVEDEEDAKIIERFKTFTTYFTGFHENRKNIYDNEEKSTAIGYRIVHENLPKFIDNMNAFEKISKALDLSEIDRDFQSELGEIKAEEFFTIEFFNQCLNQFGIDRYNTLLGGISEGENIKKKQGLNERINLYNQQLKGERKKERLPKLKVLYKQILSDSSSHSFSIDEFENDNELLESLEIFYKNELIGFNHSGVDSNIFDLVKDLLLKIDESEQSSIYLKNDKGLTEISQRIFGDWNIIKSALEEYYDEHYPPKKDTFNKKELDERSRWLKENHSIGVIEKALANYENEIVREHLKQNSAPIVSYFKSLEVDGENLIDKIYSAYGNISDLLNSSYPDEKKLVSDRTSKDKIKVFLDSLMSLLHFLKPLDVKDLGNKDSAFYGDYDFIVEQLSKLVRLYNKTRNYLTRKPYSIEKIKLNFENSTLLAGWDVNKERDNNCVIFKRQDGDRELFYLGIMDKSHNKIFTKIEEAKSDDVYQKMNYKLLPGPNKMLPKVFFSKKSIDFYAPGEELLKNYKNGTHKKGENFNLQHCHELIDFFKRSINKHEDWSQFNFKFSDTSEYEDTSFFFKEVSQQGYSITFKNIDRETIEKFVDEGKLYLFQIYNKDFSPKSKGRPNLHTLYWKMLFDERNLANTVYQLNGEAEVFYRKKSISEKDRVVHRADEPIGLKNSENSAQKSLFPYDIVKDRRFTVDKFQFHVPITLNFKSEGNERLNISVNKFLKDNPDVNIIGLDRGERHLIYLTLINQKGEILHQESLNEVMGVNYQQKLHRVEKDRTEERRNWDRIENIKELKSGYLSQVVHKISQLMVEYNAIVVMEDLNFGFKRGRIKVEKQVYQKFEKTLIDKLNYLVFKDREPEEPAGVLNALQLTNKFESFKKLGKQCGFLFYVTSDYTSKIDPATGFVNLLYPKYESVEKSQNFFRKFDNICFNSGAGYFEFDFDYSNFTDRADGTRTRWKVCTVGNERFGYNPKTKASETVNVTESLKELLLQHEIAFENGESLVESISKNTTKYFHKSLLNFLRLTLTLRHSKTGTDIDYILSPVANEEGVFFDSRNASDKMPKDADANGAYNVALKGLMVLERINAAEDLSQFKFKDMSIKNKDWLKFVQDRQG

SEQ ID NO: 76 - protein, 1271 aa, unidentified
  source 1..1271: note = Description of Unknown: Cas12 sequence
MKNLANFTNLYSLQKTLRFELKPIGKTLDWIIKKDLLKQDEILAEDYKIVKKIIDRYHKDFIDLAFESAYLQKKSSDSFTAIMEASIQSYSELYFIKEKSDRDKKAMEEISGIMRKEIVECFTGKYSEVVKKKFGNLFKKELIKEDLLNFCEPDELPIIQKFADFTTYFTGFHENRENMYSNEEKATAIANRLIRENLPRYLDNLRIIRSIQGRYKDFGWKDLESNLKRIDKNLQYSDFLTENGFVYTFSQKGIDRYNLILGGQSVESGEKIQGLNELINLYRQKNQLDRRQLPNLKELYKQILSDRTRHSFVPEKFSSDKALLRSLLDFHKEVIQNKNLFEEKQVSLLQAIRETLTDLKSFDLDRIYLTNDTSLTQISNFVFGDWSKVKTILAIYFDENIANPKDRQRQSNSYLKAKENWLKKNYYSIHELNEAISVYGKHSDEELPNTKIEDYFSGLQTKDETKKPIDVLDAIVSKYADLESLLTKEYPEDKNLKSDKGSIEKIKNYLDSIKLLQNFLKPLKPKKVQDEKDLGFYNDLELYLESLESANSLYNKVRNYLTGKEYSDEKIKLNFKNSTLLDGWDENKETSNLSVIFRDTNNYYLGILDKQNNRIFESIPEIQSGEETIQKMVYKLLPGANNMLPKVFFSEKGLLKFNPSDEITSLYSEGRFKKGDKFSINSLHTLIDFYKKSLAVHEDWSVFNFKFDETSHYEDISQFYRQVESQGYKITFKPISKKYIDTLVEDGKLYLFQIYNKDFSQNKKGGGKPNLHTIYFKSLFEKENLKDVIVKLNGQAEVFFRKKSIHYDENITRYGHHSELLKGRFSYPILKDKRFTEDKFQFHFPITLNFKSGEIKQFNARVNSYLKHNKDVKIIGIDRGERHLLYLSLIDQDGKILRQESLNLIKNDQNFKAINYQEKLHKKEIERDQARKSWGSIENIKELKEGYLSQVVHTISKLMVEHNAIVVLEDLNFGFKRGRQKVERQVYQKFEKMLIEKLNFLVFKDKEMDEPGGILKAYQLTDNFVSFEKMGKQTGFVFYVPAWNTSKIDPKTGFVNFLHLNYENVNQAKELIGKFDQIRYNQDRDWFEFQVTTDQFFTKENAPDTRTWIICSTPTKRFYSKRTVNGSVSTIEIDVNQKLKELFNDCNYQDGEDLVDRILEKDSKDFFSKLIAYLRILTSLRQNNGEQGFEERDFILSPVVGSDGKFFNSLDASSQEPKDADANGAYHIALKGLMNLHVINETDDESLGKPSWKISNKDWLNFVWQRPSLKA

SEQ ID NO: 77 - protein, 816 aa, unidentified
  source 1..816: note = Description of Unknown: Cas12 sequence
MNLIENETKSEEIKSKLDSIMEIMHWTKMFIIEEEIEKDVNFYNEIEEIYDELQPLVTIYNRIRNYVTQKPYSEEKIKLNFGIPTLANGWSKTKEYDNNAIIMIRDGKYYLGIFNAKNKPDKKIMEGHQSEENGDYKKMIYRLLPGPNKMLPKVFMSKTGIAEYKPSQYILECYEQNKHIKSDKNFDIKFCRDLIDFFKTSINRHPEWSKFNFKFSETSEYEDISTFYREVEKQGYKIEWTYISEKEIKELDENGQLYLFQIYNKDFSEKSKGKENLHTMYLKNLFSEENLKNIVLKLNGEAEVFFRKSSIKKPIIHKKGSVLVNKTYNENGERKSIPEEQYTEIYKYLNSIGTNELSEKSKKLMEEGKVEYYKANYDIVKDYRYSVDKFFIHLPMTINFKAAGFSPINNIALKSIALKEDMHIIGIDRGERNLIYVSVIDTKGNIVEQRNFNIVNGIDYKEKLKQKELDRDNARKNWKEIGKIKDLKEGYLSLVVHEIAKLVVKYNAIITMEDLNQGFKRGRFKVERQVYQKFETMLINKLNYLVDKDLAVDQEGGLLRGYQLTYIPESLKVLGRQCGYIFYVPVAYTSKIDPTTGFVAIFNYKGMTDKDFVTSFDSIKYDDERGLFAFEFDYENFVTHKVEMARNKWTVYTYGERIKRKFKNGLWDTAEKVDLTYQMRSILEKYEIEYNKGQDILEQIEELDEKAQNGICKEIKYLVKDIVQMRNSLPDNAVEDYDAIISPVINNNGEFFDSTRGDEDKPLDADANGAYCIALKGLYEVMQIKKNWNEETEFPRKELKIRHQDWFDFIQNKRYL

SEQ ID NO: 78 - protein, 869 aa, unidentified
  source 1..869: note = Description of Unknown: Cas12 sequence
MENRYQVLQGLTAAQKKAAAAAKKRSSFSIVELNAATRSRVPDEKYVPVQNYFSAMGKVCSQGEPKRENFVTRICAAYQELEEYIPSIRKSLLQEKRATELIKNYLDAVNDLLRFIKPLLGRGNETDKDANFYGEFSFLTDCLFAIVPLYNEVRNYLTQKPYSTEKFKLNFRGSTLLNGWDKNKERDNLGVILRKEGKYFLAIMNKKHNTLFTEGKLQQHTGGECYQKMEYKLIPGSKMLPKVFFSKKGISTFQPSEELLLNYRIGTYKKGEKFNLEHLHKLIDFYKHSIAVHEDWSKFDFHFSDTSSYRDISGFYKEVEQQGYKLTFRNVSVSYINRLVEEGKLYLFQIYNKDFSEYSKGTPNLHTLYWKMLFDPENLKDVVYKLSGEAEVFFRKKSLDVSHPTHPKNEPIEKKNINNKGEKSLFSYDLIKDRRFTVDKFQFHVPITMNFKGEQGDRVNQMVQSYVRNNKGLNVIGIDRGERNLLYLVVINEHGEILEQFSLNEIRNAYNGKEHKIDYHTLLEERSKKRQDARQSWQTIEGIKDLKTGYLSQVIHVITQLMVKYNAIVVLEDLNFGFKSSRQKFEQSVYQQFERKLIDKLNFLVNKKAAPNEVGGLLNAYQLTAPLGNSRKMGKQNGFLFYVPAWHTSKIDPRTGFVNLLDTRYENVAKAKEFFAKFASITYNPEKKWFEFAFDYKAFGNRADGSRTKWTICSYGERIETFRNPENNNQWDTKSVPLTERLTELFSKYGIDYTTNLKEQILNQTDKAFFVELLGALRLTLQLRNSRKSTGEDFLFSPVADENGCFFDSREANDNEPKDADANGAYHIALKGLWVLDTIRNTEEGKNPKLAITNKEWLSFAQAKPFAHE

SEQ ID NO: 79 - protein, 884 aa, unidentified
  source 1..884: note = Description of Unknown: Cas12 sequence
MSDSYDELTKAQKEKQEKRKHVALTEVVAALEKYTIALDNGHEHKNAVNTFKNYFQNYFFHFDTDKKKTAKTLDCQIKDEYNGLKGILNTPWDKNKKLQQDKKLVQQIKSFLDSIQELLWFIKPLVLTDNTLEKDERFYGEFMPLYDEISNIIKLYNKIRNYLTKKPYSIEKYKLNFENGSLLSGWDVNKEKDNTSVLLCKDNQYYLAIMHIDHNKVFELDELIKHAGKGYQKINYKLLPGANKMLPKVFFSGKNISYYDPSKEILKIRNYGTHTKNGDPQPGFSKRDFSVDDCRKMIDFFKNSIAKHEDWKNFDFKFQPTKNYNSIDEFYREVEEQGYKITYSNVSEDYIDSLVEYGKIYLFHIYNKDFSDKRDESKKHTDNMHTLYWKALFDAKNLKDVVYKLNGEAEIFYRKKSIDIKKPTHEKGKPIDNKNPNARKKTSVFKYDLIKDKRFTVDKFFFHVPITLNFKSKSGYLSNDDVNAAIKKNNDIKIIGLDRGERNLIYLSLINSKGEIAYQESLNVVSTDKGFDVNYHKLLDDKEGNRDEARKNWDKIENIKELKAGYLSQVIHKIAKLMIDNNAIVVMEDLNFGFKRGRFKVEKQIYQKFEKMLIDKLNYLVFKNVHPEQAGGLYKAYQLTAQFESFKKLGKQSGFLFYIPAWNTSKIDPTAGFVDFLKPRYESVTQAKSFLQRFDKINYNKTKDYFEFAFDYKNFTDKANDTKTDWVVCTYGTERYYYDVRTKTTQKIDITAELKKLLEKSEINYLNGKDIKELIIAVDSKEFHSALLKYLAIVLALRYSDSQSGRDFILSPVANEQGHFFNSDKTDDTLPKDADANGAYHIALKGLWAINQIRKTKNGDKLKLTISNKDWLNFVQKKEYRKGV

SEQ ID NO: 80 - protein, 1250 aa, unidentified
  source 1..1250: note = Description of Unknown: Cas12 sequence
MQTLFENFTNQYPVSKTLRFELIPQGKTKDFIEQKGLLKKDEDRAEKYKKVKNIIDEYHKDFIEKSLNGLKLDGLEKYKTLYLKQEKDDKDKKAFDKEKENLRKQIANAFRNNEKFKTLFAKELIKNDLMSFACEEDKKNVKEFEAFTTYFTGFHQNRANMYVADEKRTAIASRLIHENLPKFIDNIKIFEKMKKEAPELLSPFNQTLKDMKDVIKGTTLEEIFSLDYFNKTLTQSGIDIYNSVIGGRTPEEGKTKIKGLNEYINTDFNQKQTDKKKRQPKFKQLYKQILSDRQSLSFIAEAFKNDAEILEAIEKFYVNELLHFSNEGKSTNVLDAIKNAVSNLESFNLTKMYFRSGTSLTDVSRKVFGEWSIINRALDNYYATTYPIKPREKSEKYEERKEKWLKQDFNVRLIQTAIDEYDNETVKGKNSGKVIADYFAKFCDDKETDLIQKVNEGYIAVKDLLNTPYPENEKIGSNKDQVKQIKAFMDSIMDIMHFVRPLSLKDTDKEKDETFYSLFTPLYDHLTQTIALYNKVRNYLTQKPYSTEKIKLNFENSTLLGGWDLNKETDNTAIILRKDNLYYLGIMDKRHNRIFRNVPKADKKDFCYEKMVYKLLPGANKMLPKVFFSQSRIQEFTPSAKLLENYANETHKKGDNFNLNHCHKLIDFFKDSINKHEDWKNFDFRFSATSTYADLSGFYHEVEHQGYKISFQSIADSFIDDLVNEGKLYLFQIYNKDFSPFSKGKPNLHTLYWKMLFDENNLKDVVYKLNGEAEVFYRKKSIAEKNTTIHKANESIINKNPDNPKATSTFNYDIVKDKRYTIDKFQFHIPITMNFKAEGIFNMNQRVNQFLKANPDINIIGIDRGERHLLYYALINQKGKILKQDTLNVIANEKQKVDYHNLLDKKEGDRATARQEWGVIETIKELKEGYLSQVIHKLTDLMIENNAIIVMEDLNFGFKRGRQKVEKQVYQKFEKMLIDKLNYLVDKNKKANELGGLLNAFQLANKFESFQKMGKQNGFIFYVPAWNTSKTDPATGFIDFLKPRYENLNQAKDFFEKFDSIRLNSKADYFEFAFNFKNFTEKADGGRTKWTVCTTNEDRYAWNRALNNNRGSQEKYDITAELKSLFDGKVDYKSGKDLKQQIASQESADFFKALMKNLSITLSLRHNNGEKGDNEQDYILSPVADSKGRFFDSRKADDDMPKNADANGAYHIALKGLWCLEQISKTDDLKKVKLAISNKEWLEFVQTLKG

SEQ ID NO: 81 - protein, 810 aa, unidentified
  source 1..810: note = Description of Unknown: Cas12 sequence
MQLTDNLSDKYKEAAPLLNENYSNEKGLKNDDKSISLIKNFLDAIKEIEKFIKPLSETNITGEKNDLFYSQFTPLLDNISRIDILYDKVRNYVTQKPFSTDKIKLNFGNSQLLNGWDRNKEKDCGAVWLCKDEKYYLAIIDKSNNSILENIDFQDCDESDCYEKIIYKLLPGPNKMLPKVFFSEKCKKLLSPSDEILKIRKNGTFKKGDKFSLDDCHKLIDFYKESFKKYPNWLIYNFKFKKTNEYNDIREFYNDVASQGYNISKMKIPTSFIDKLVDEGKIYLFQLYNKDFSPHSKGTPNLHTLYFKMLFDERNLEDVVYKLNGEAEMFYRPASIKYDKPTHPKNTPIKNKNTLNDKKTSAFPYDLIKDKRYTKWQFSLHFPITMNFKAPDRAMINDDVRNLLKSCNNNFIIGIDRGERNLLYVSVIDSNGTIIYQHSLNIIGNKFKGKTYKTNYREKLATREKDRTEQRRNWKAIESIKELKEGYISQAVHVICQLVVKYDAIIVMEKLTEGFKRGRTKFEKQVYQKFEKMLIDKLNYYVDKKLDPDEEGGLLHAYQLTNKLESFDKLGTQSGFIFYVRPDFTSKIDPVTGFVNLLYPRYENIDKAKDMISRFDEIRYNAGEDFFEFDIDYDKFPKTASDYRKKWTICTNGERIEAFRNPANNNEWSYRTIILAEKFKELFDNNSINYRDSDDLKAEILSQTKGKFFEDFFKLLRLTLQMRNSNPETGEDRILSPVKDKNGNFYDSSKYDEKSKLPCDADANGAYNIARKGLWIVEQFKKADNVSTVEPVIHNDKWLKFVQENDMTNN

SEQ ID NO: 82 - protein, 875 aa, unidentified
  source 1..875: note = Description of Unknown: Cas12 sequence
MLPNEKERNEFKNSNAKQYIREISNIITDTETAHLEYDEHISLIESEEKADEMKKRLDMYMNMYHWAKAFIVDEVLDRDEMFYSDIDDIYNILENIVPLYNRVRNYVTQKPYNSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYNDISEFYREVEMQGYRIDWTYISEADINKLDEEGKIYLFQIYNKDFAENSTGKENLHTMYFKNIFSEENLKDIIIKLNGQAELFYRRASVKNPVKHKKDSVLVNKTYKNQLDNGDVVRIPIPDDIYNEIYKMYNGYIKENDLSEAAKEYLDKVEVRTAQKDIVKDYRYTVDKYFIHTPITINYKVTARNNVNDMAVKYIAQNDDIHVIGIDRGERNLIYISVIDSHGNIVKQKSYNILNNYDYKKKLVEKEKTREYARKNWKSIGNIKELKEGYISGVVHEIAMLMVEYNAIIAMEDLNYGFKRGRFKVERQVYQKFESMLINKLNYFASKGKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMSKTQWTVYTNGERLQSEFNNARRTGKTKSINLTETIKLLLEDNEINYADGHDVRIDMEKMDEDKNSEFFAQLLSLYKLTVQMRNSYTEAEEQEKGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 83 - protein, 1238 aa, unidentified
  source 1..1238: note = Description of Unknown: Cas12 sequence
MSNLYSNLHNLYPVQKTLRFELKPQGKTKENMEKAGILKADEHRAEVYGKVKKYCDEYHKTFIDRCLSNIELNEIDKYYELYSINNRDDKQKEELDQLETGLRKQISDAFKKSAEYKGLFQKDMITSYLVTMYKENQEKMQDIGEFNRFTTYFTGYNKNRENMYSEEDKSTAISYRLINENLPTFIDNIKIYKKIVSLMPENIEKIYKDLEEYIQVNSVDEIFNISYYNDVLTQRGIECYNILISGRTKNDGDKIKGLNEYINEFNQTHNEKIPKLQELYKQILSDAESASFKVDIIENDKELLNLIEVYYANILPTLNKIEDLFTRISNYNLELILVNNDGSLSTLSNMVFNEWSYIKGIISQKYDAEYSGKEKYGTEKYAQKKQEYLKKQKIYSLKFLNDCIGNNAICEYLKNYIIQNKNIETIKEDYNEVQNIKAEDDTKELIKDEKSIEKIKKFLDDVKSLQEFVKLVIPKDRTVEKDAKFYSELTPYYEKIKEIIPLYNKVRNYVTQKPYSTEKIKLNFECPTLLNGWDANKEEANLGVILLKEGKYYLGIMNPYCKKIFEVYEKDSNEQNNYKKMEYKLLPGPNKMLPKVFFSNSRIEEFNPSKELQEKYNKGYHKKGKDFDINFCHELIDFYKQSLNKHEDWKKFNFKFKDTSEYNDISEFYREVEEQGYKIEYTEYSEKYINELVDRGELYLFQIYNKDFSEYSKGKENLHTLYWKAVFDPDNIMNPVYKLNGNAEIFYRKKSLEMKVTHPANQPIANKNISTIEAGRSTSTFKYDLIKDKRYTMDKFQFHVPITVNFKSERLFNINQIVNKYLKYNDDIHVIGIDRGERNLLYVCVIDKNEKIVYQKSLNEIVSEYNNNRYTTDYHGLLDRKEKEREIAREDWKNIENIKELKEGYMSQIIHILVELMKKYNAIIVIEDLNKGFKNSRIKVEKQVYQKFEKMFIDKLNYLVFKDEDKMDEGGVLNAYQLTNKFESFTKLGKQSGILYYIPAWCTSKIDPTTGFINRFYLKYENFDKSKEFVNRIDDIRYNEKENLFEFDIDYSKFTDRLNDTKNKWTLCSYGERILTQKNANGEWFDRRIQLSIEFKNLFEKYVINLNNIKDSILKLDKDNIEFYKGNGENLGFIQLFKLMVQMRNSLTGKEEDNLISPVKNQHGKFFNTSERVEGLPIDADANGAYNIARKGFMLVEQMKNVEDEKLNKIKYNITEKEWLNYVQNRGMWWKRQYLYHI

SEQ ID NO: 84 - protein, 1262 aa, unidentified; identical to SEQ ID NO: 74
  source 1..1262: note = Description of Unknown: Cas12 sequence

SEQ ID NO: 85 - protein, 1140 aa, unidentified
  source 1..1140: note = Description of Unknown: Cas12 sequence
MYKDKTDKTKIIDSDLIKFINIAESTQLDSMSQDEAKELVKEFWGFTTYFVGFYDNRKNMYTAEEKSTGIAYRLVNENLPKFIDNMEAFKKAIARPEIQANMEELYSDFSEYLNVESVQEMFQLDYYNMLLTQKQIDVYNAIIGGKTDDEHDVKIKGINEYINLYNQQHKDDKLPKLKALFKQILSDRNAISWLPEEFNGDQEVLNAIKDCYERLSENVLGDKVLKSLLGSLSDYSLDGIFIRNDLQLTDISQKMFGNWCVIQNAIMQNIKHVAPARKHKESEEDYEKRIAGIFKKVDSFSISFINDCLNEADPNNAYFVENYFATFGAVNTPTMQRENLFALVQNAYTEVAALLHSDYPTAKHLAQDKVNVAKIKALLDAIKSLQHFVKPLLGKGDESDKDERFYGELASLWAELDTVTPLYNMIRNYMTRKPYSQKKIKLNFENPQLLGGWDANKEKDYATIILRRDGLYYLAIMNKESKKLLGKAMPSDGECYEKMVYKLLPGANKMLPKVFFAKSRMEDFKPSKELVEKYNNGTHKKGKNFNIQDCHNLIDYFKQSISKHEDWGKFGFNFSDTSTYEDLSGFYREVEQQGYKLSFARVSVSYISQLVEEGKMYLFQIYNKDFSEYSKGTPNMHTLYWKALFDERNLADVVYKLNGQAEMFYRKKSIENTHPTHPANHPILNKNKDNKKKESLFDYDLIKDRRYTVDKFMFHVPITMNFKSSGSENINQDVKAYLRHADDMHIIGIDRGERHLLYLVVIDLQGNIKEQYSLNEIVNEYNGNTYHTNYHDLLDVCEEERLKARQSWQTIENIKELKEGYLSQVIHKITQLMVKYHAIVVLEDLNMGFMRGRQKVEKQVYQKFEKMLIDKLNYLVDKKADASVSGGLLNAYQLTSKFDSFQKLGKQSGFLFYIPAWNTSKIDPVTGFVNLLDTRYQNVEKAKVFFSKFDAIRYNKDKDWFEFNLDYDKFGKKAEGTRTKWALCTRGMRIDTFRNKEKNSQWDNQEVDLTAEMKSLLEHYYIDIHGNLKDAISAQTDKAFFTGLLHILKLTLQMRNSITGTETDYLVSPVADENGIFYDSRSCGDELPENADANGAYNIARKGLMMIEQIKDAKDLDNLKFDISNKSWLNFAQQKPYKNE

SEQ ID NO: 86 - protein, 832 aa, unidentified
  source 1..832: note = Description of Unknown: Cas12 sequence
MNEADPNNAYFVENYFATFGAVNTPTMQRENLFALVLNAYTEVASLLHSYYPAEKNLAQDKANVAKIKALLDAIKSLQHFVKPLLGKGDESDKDERFYGELASLWAELETVTPLYNMIRNYMTRKPYSQKKIKLNFENPQLLGGWDANKEKDYATIILRRNGLYYLAIMDKDSRKLLGKAMPSDGECYEKMVYKLLPGANKMLPKVFFAKSRMEDFKPSKELVEKYNNGTHKKGKNFNIQDCHNLIDYFKQSISKHEDWGKFGFNFSDTSTYEDLSGFYREVEQQGYKLSFARVSVSYISQLVEEGKMYLFQIYNKDFSEYSKGTPNMHTLYWKALFDERNLADVVYKLNGQAEMFYRKKSIENTHPTHPATHPILNKNKDNKKKESLFEYDLIKDRRYTVDKFMFHVPITMNFKSVGSENINQGVKEYLHHADDMHIIGIDRGERHLLYLVVIDLQGNIKEQYSLNEIVNEYNGNTYHTNYHDLLDAREDERLKARQSWQTIENIKELKEGYLSQVIHKITQLMVKYHAIVVLEDLNMGFMRGRQKVEKQVYQKFEKMLIDKLNYLVDKKADASVSGGLLNAYQLTSKFDSFQKLGKQSGFLFYIPAWNTSKIDPVTGFVNLLDARYQNVEKAKAFFSKFDAIRYNKDKDWFEFNLDYDKFGKKAEGTRTKWTLCTRGMRIDTFRNKEKNSQWDNQEVDLTAELKSLLEHYYIDIHGNLKEAISTQTDKAFFTGLLHILKLTLQMRNSITGTETDYLVSPVADENGIFYDSRTCGDELPENADANGAYNIARKGLMMIEQIKNAEDLGNLKFDISNKAWLNFAQQKPYKNG

SEQ ID NO: 87 - protein, 831 aa, unidentified
  source 1..831: note = Description of Unknown: Cas12 sequence
MDNNNLHAVDGYFATLGAVNTPTMQRENLFALIQNAYTDISDLLDTPYPENKNLAQDKTNVAKVKALLDAIKSLQHFVKPLLGMGDESDKDERFYGELASLWTELDTVTPLYNMIRNYMTRKPYSEKKIKLNFENPQLLGGWDANKEKDYATIILRRNGMYYLAIMDKDSKKLLGKTMPSDGECYEKMVYKLLPGANKMLPKVFFAKSRINDFKPSKKIVENYNNGTHKKGKNFNINDCHDLIDYFKQSIDKHEDWSKFGFNFSDTSTYEDLSGFYREVEQQGYKLSFTNISVSFIDKLVDEGKMYLFQIYNKDFSDYSKGTPNMHTLYWKALFDERNLADVVYKLNGEAEMFYRKKSINNTHPTHHANHPIQNKNKDNKKKESVFEYDLVKDRRYTEDKFLFHVPITMNFNSVGAENINQQVRKYLQQADDTHIIGIDRGERHLLYLVVIDMQGNIKEQFSLNEIVNEYNGNTYRTNYHDLLDVRADKRLKASQSWQTIENIKELKEGYLSQAIHKITQLMVKYHAVVVLEDLNKGFMRGRQKVEKQVYQKFEKMLIDKLNYLVDKHKDANETGGLLHALQLTSEFKNFKKSEYQNGFLFYIPAWNTSKIDPVTGFVNRFDTRYTNAVEAQKFFRKFDEIRYNEEKDWFEFEFDYDKFTQKAHGTRTRWTLCTHGKRLRSFRNPAKQYNWDSEVVALTDEFKRILGEAGIDIHENLKDAISNLEGKRRKYLEPLMQFMKLLLQLRNSRKNPEEDYILSPVADENGVFYDSRSCGDKLPENADANGAYNIARKGLMLIRQIKEAKELGKVKYDISNKAWLNFAQQKPYKNE

SEQ ID NO: 88 - protein, 1177 aa, unidentified
  source 1..1177: note = Description of Unknown: Cas12 sequence
MQKKAFEENQQNLRSIIAKKLTEDKAYANLFGKNLLESYKDKTDKTKIIDSDLIKFINTAESTQLDSMSQDEAKEIVKEFWGFTTYFVGFFDNRKNMYTAEEKSTGIAYRLINENLPKFIDNMEAFKKAIARPEIQADMEELYSNFSEYLNVESIQEMFLLDYYNMLLTQKQIDVYNAIIGGKTDDEHDVKIKGINEYINLYNQQHKDDKLPKLKALFKQILSDRNAISWLPEEFNSDQEVLNAIKDCYERLSENVLGDKVLKSMLGSLADYSLDGIFIRNDLQLTDISQKMFGNWSVIQNAIMQNIKHVAPARKHKESEEEYENRIAGIFKKADSFSISYIDACLNETDPNNAYFVENYFATLGAVDTPTMQRENLFALVQNAYTEITALLHSDYPTEKNLAQDKANVAKIKALLDAIKSLQHFVKPLLGKGDESDKDERFYGELASLWAELDTMTPLYNMIRNYMTRKPYSQKKIKLNFENPQLLGGWDANKEKDYATIILRRNGLYYLAIMNKDSKKLLGKAMPSDGECYEKMVYKLLPGANKMLPKVFFAKSRMEDFKPSKELVEKYNNGTHKKGKNFNIQDCHNLIDYFKQSIDKHEDWSKFGFKFSDTSTYEDLSGFYREVEQQGYKLSFARVSVSYINQLVEEGKMYLFQIYNKDFSEYSKGTPNMHTLYWKALFDERNLADVVYKLNGQAEMFYRKKSIENTHPTHPANHPILNKNKDNKKKESLFEYDLIKDRRYTVDKFMFHVPITMNFKSVGSENINQDVKAYLRHADDMHIIGIDRGERHLLYLVVIDLQGNIKEQFSLNEIVNDYNGNTYHTNYHDLLDVREDERLKARQSWQTIENIKELKEGYLSQVIHKITQLMVKYHAIVVLEDLNMGFMRGRQKVEKQVYQKFEKMLIEKLNYLVDKKADASVSGGLLNAYQLTSKFDSFQKLRKQSGFLFYIPAWNTSKIDPITGFVNLLDTRYQNVEKAKAFFSKFDAIRYNKDKEWFEFDLDYDKFGRKAEGTRTKWTLCTRGMRIDTFRNKEKNSQWDNQEIDLTAEMKSLLEHYYIDIQGNLKEAICTQTDKAFFTGLLHILKLTLQMRNSITGTQTDYLVSPVADENGIFYDSRSCGDSLPENADANGAYNIARKGLMLIEKIKNAEDLDTIKFDISNKAWLNFAQQKPYKNG

SEQ ID NO: 89 - protein, 814 aa, unidentified
  source 1..814: note = Description of Unknown: Cas12 sequence
MLENEQEAEKIKNKLDDIMNIYHWIKIFLVDEEIEKDMDFYSEIEDIYEELSPLVSLYNRVRNYVTQKPYSQEKMKLNFGSPTLADGWSKSKEFSNNAIIMLKDGKYYIGIFNIRNKPNKEVIEGRNNRINDSDYKKMVYRLLPGANKMLPKVMFSKKGIEYYNPSQYILSGYNSKKHIKSNENFDINFCHDLIDFFKESINKNEEWKNFDFKFSDTESYNDISEFYREVEQQGYKIEWVYISEQDIEQLEKNGQLYIFQIYNKDFAKKSIGNKNLHTMYLENLFSEENLKDVVLKLNGEAEIFFRKSSIKKPIVHKAGSILVNKCIEDETGNKVSFPDDIYNEIYQYMNGMTDVLSERAQNYYEKVKHSVSKQDIVKDYRYTVDKYFIHLPITINFKASSFMPINDIALKYIAKRDDIHIIGIDRGERNLIYVSVIDLQGNIVYQKNYNVVNGYDYKAKLRETEIQRDNARKNWKEIGKIKQLKEGYLSLVVHEIAQLIVKYNAIVVMEDLNMGFKRGRFKVERQVYQKFENMLINKLNYLVDKNKKVDEDGGLLRGYQLTYVPGQKEHVGKQCGFIFYVPAAYTSKIDPTTGFVSIFNNKVNAKEFVTKFDSIKYNKNMKMFELKFDYNNFETYNITLAKSKWTIYTNGIRLKREYNNGRWNKITEVDLTKEMANTLKKYDIEFENNEEILKSISQLDEKNQRNICNEIKEIIKLIVQLRNSMPDNGSKDNEYDKIISPVLNENNYFYDSSEVCDNSAPENADANGAYCIAMKGLYQVIQIKENWSQDSNPKNILGIKHYEWFDFMQNKRYL

SEQ ID NO: 90 - protein, 1262 aa, unidentified
  source 1..1262: note = Description of Unknown: Cas12 sequence
MAKNTIFTQFTGLYPVSKTLRFELKPMGKTLEKIKETGVIENDKKRHNDYFDAKKIIDKYHKYFIDAALSKFSRIDWNPLKEAIEGSLDRSDASKKKLEKTQTEFRKKIAKALTTHDHYKELTASTPKDLFLKVFPDHFGKQPAIDTFDGFSSYFTGFQENRQNIYSDEAISTAIPYRLVHDNFPKFLSNIEVYKTLKDNAPSVLSDAENELKDFLNGKPLANIFELNAYNDVLTQSGIDFFNQVIGGISGEGGEKKTRGINEFSNLYRQQHPEFAQKRLATKMIPLYKQILSDRETKSFILESYSTDSQVQESVKEFFESQILNCDIAGRKVNVLNELTSLIKRIAEFDLGSIYINQEELSNISLELFKSWNTINAVLFKNAENRIGSAEKAANKKKIDAWMKSNEFSIATLNLAIAESDSEEISRVKIESYWNDFEAKVQSILCGDNRRNLDEFLSATFNENNALREDSEIIGKLKAFLDALIEIMHSIKPLISDAENRDLSFYNELMPLYDQLSLVVPLYNKIRNYATQKLTESEKFKLNFDNPTLADGWDQNKEDANTAILLLKNGLYYLGIMNAKNKPKIKDFKTSESEDCYDKMVYKLLPGPNKMLPKVFFSEKGLATFKPPKDILDGYNAGKHKKGDLFDIGFCHQLIDFFKESIAKHPDWKKFDFKFSDTSSYEDISGFYKEVTDQGYKITFSKIPTSQIDEWVNEGKLFLFQIYNKDFAPGAKGSPNLHTLYWKSVFSPENLKDVVVKLNGEAELFYRPSSVKKPYSHKVGEKLVNRIGKDGLPLPESVFGELFRYFNGKLDGELSDEAKRYLDVAVVKDVKHEIVKDRRYTQDKFEFHVPLTLNFKADSKNEYMNERVRHFLKDNPDVNIIGIDRGERHLLYMTLINQKGEILKQKSFNIVESVNYQAKLVQREKERDAARKSWSSVGKIKDLKEGFLSQVIHEITTTMIENNAIVVLEDLNFGFKRGRFCVERQVYQKFEKMLIDKLNYLVFKNKPEGDVGGVLKGYQLAEKFDSFQKLGKQSGFLFYIPAAYTSKIDPTTGFANLFNMTELTSAEKKKDFLSHFDDITYDGKNDRFLFGFDYKNFKCFQTDFIKKWTVYTQGKRIVYDKESKSAKEIFPVEIIKAALAKQNIALTDQLDVLSAINSVEASPKSASFFGNICYAFEKTLQMRNSIPNTDEDYLVSPVMNKRGEFYDSRSCDDALPQNADANGAYHIALKGLYLIKNVFDAGGKDLKISHEDWFKFAQSRNC

SEQ ID NO: 91 - protein, 791 aa, unidentified
  source 1..791: note = Description of Unknown: Cas12 sequence
MRPVLQLTDTEDKLSQNKPAVGKIKALLDAFKDLQHFIKPLLGSGEENEKDELFYGAFQLIWDELDTVTPLYNKVRNWLTRKPYSTEKIKLNFDNAQLLGGWDVNKEPDCTGVLLRKDGFYYLGIMNKKSNRIFDADVTPADGICYEKIDYKLLPGANKMLPKVFFSKSRIDEFAPSEAILSSYKRGTHKKGADFSLSDCHRLIDFFKASINKHEDWSKFGFQFSDTKTYEDISGFYREVEQQGYMLSSHQVSEAYINQMVEEGKLFLFRIWNKDFSEYSKGTPNMHTLYWRMLFDERNLADVVYKLNGQAEVFYRKASIKAENQIMHPAHHPIENKNTLNEKRSSTFDYDLVKDRRYTVDKFQFHVPITINFKAIGQTNVNPIVHETIRRGGFTHVIGIDRGERHLLYLSLIDLKGHIVKQMTLNEIINEYNGLAHKTNYYDLLVKREGERTTARRSWDTIENIKELKEGYLSQVIHIISKMMVEYNAIVVLEDLNMGFMRGRQKIERQVYEKFEKMLIDKLNCYIDKQADSQSEGGLLHPIQLANKFESFRKLGKQSGCLFYIPAWNTSKIDPVTGFVNLFDTRYETREKAKLFFSHFQRICFNAEKDWFEFSFDYNDFTTKAEGTRTQWTLCSYGTRIRNFRNPLQNHQWDDEEIVLTEAFKALFDKYDIDIHANLKEAINAQTDAQFFKDLMGLMKLLLQMRNSKTNSEVDYLLSPVADEHGRFFDSRAGAGSLPDNADANGAYNIARKGLWVIRKIQETPEGEKLSLAITNKEWLEFAQTKPYLND

SEQ ID NO: 92 - protein, 1262 aa, unidentified
  source 1..1262: note = Description of Unknown: Cas12 sequence
MNLNTYFSQFTGLYPVSKTLRFELKPMGKTLEKIKETGIIENDKKRHNDYFDAKKIIDKYHKYFIDAALSKFPCIDWNPLKEAIERSLDRSDASKKKLEKTQTEFRKKIAKALTTHGHYKELTASTPKDLFLKVFPDHFGKQPAIDTFDGFSSYFTGFQENRQNIYSDEAISTAIPYRLVHDNFPKFLSNIEVYNILKDNAPSVLSDAENELKDFLNGKPLANIFELNAYNDVLTQSGIDFFNQVIGGFSGEGGEKKTRGINEFSNLYRQQHPEFAQKRLATKMIPLYKQILSDRETKSFILESYSTDSQVQESVKEFFESQILNCDIAGRKVNVLKELSSLIKRITEFDLGSIYVNQEELSSISLELFKSWNTINAILFKNAENRIGSAEKAANKKKIDAWMKSNEFSIATLNLAIAESDSEEISRVKIESYWNNFEAKVQSILCGDNRRNLDEFISATFNENNALREDSKVIEKLKAFLDALIEIMHSIKPLISDAENRDLSFYNELMPLYDQLSLVVPLYNKIRNYATQKLTESEKFKLNFDNPTLADGWDQNKEEANTAILLLKNGLYYLGIMNAKNKPKIKDFKTSESEDCYDKMVYKLLPGPNKMLPKVFFSEKGLATFKPPKDILDGYNAGKHKKGDLFDIGFCHQLIDFFKESIAKHPDWKKFDFKFSDTSSYEDISGFYKEVTDQGYKITFSKIPTPQIDEWVNEGKLFLFQIYNKDFAPGAKGSPNLHTLYWKSVFSPENLKDVVVKLNGEAELFYRPSSVKKPYSHKVGEKLVNRIGKDGLPLPESVFGELFRYFNGKLDGELSDEAKRYLDVAVVKDVKHEIVKDRRYTQDKFEFHVPLTLNFKADSKNEYMNERVRHFLKDNPDVNIIGIDRGERHLLYMTLINQKGEILKQKSFNIVESVNYQAKLVQREKERDTARRSWSSVGKIKDLKEGFLSQVIHEITTTMIENNAIVVLEDLNFGFKRGRFCVERQVYQKFEKMLIDKLNYLVFKNKPEGDVGGVLKGYQLAEKFDSFQKLGKQSGFLFYIPAAYTSKIDPTTGFANLFNMTELTSAEKKKEFLSHFEDITYDGKNDRFLFSFDYKKFKCFQTDYIKKWTVYSQGKRIVYDKESKSAKAISPVEIIKAALAKQNIALTDQLDVLSAINSVEASRETASFFGDICYAFEKTLQMRNSIPNTDEDYLVSPVMNKKGEFYDSRSCGDSLPKNADANGAYHIALKGLYLIKNVFDAGGKDLKISHEDWFKFAQSRNR

SEQ ID NO: 93 - protein, 1250 aa, unidentified
  source 1..1250: note = Description of Unknown: Cas12 sequence
MQTLFENFTNQYPVSKTLRFELIPQGKTKDFIEQKGLLKKDEDRAEKYKKVKNIIDEYHKDFIEKSLNGLKLDGLEEYKTLYLKQEKDDKDKKAFDKEKENLRKQIANAFRNNEKFKTLFAKELIKNDLMSFACEEDKKNVKEFEAFTTYFTGFHQNRANMYVADEKRTAIASRLIHENLPKFIDNIKIFEKMKKEAPELLSPFNQTLKDMKDVIKGTTLEEIFSLDYFNKTLTQSGIDIYNSVIGGRTPEEGKTKIKGLNEYINTDFNQKQTDKKKRQPKFKQLYKQILSDRQSLSFIAEAFKNDTEILEAIEKFYVNELLHFSNEGKSTNVLDAIKNAVSNLESFNLTKIYFRSGTSLTDVSRKVFGEWSIINRALDNYYATTYPIKPREKSEKYEERKEKWLKQDFNVSLIQTAIDEYDNETVKGKNSGKVIVDYFAKFCDDKETDLIQKVNEGYIAVKDLLNTPYPENEKLGSNKDQVKQIKAFMDSIMDIMHFVRPLSLKDTDKEKDETFYSLFTPLYDHLTQTIALYNKVRNYLTQKPYSTEKIKLNFENSTLLGGWDLNKETDNTAIILRKENLYYLGIMDKRHNRIFRNVPKADKKDSCYEKMVYKLLPGANKMLPKVFFSQSRIQEFTPSAKLLENYENETHKKGDNFNLNHCHQLIDFFKDSINKHEDWKNFDFRFSATSTYADLSGFYHEVEHQGYKISFQSIADSFIDDLVNEGKLYLFQIYNKDFSPFSKGKPNLHTLYWKMLFDENNLKDVVYKLNGEAEVFYRKKSIAEKNTTIHKANESIINKNPDNPKATSTFNYDIVKDKRYTIDKFQFHVPITMNFKAEGIFNMNQRVNQFLKANPDINIIGIDRGERHLLYYTLINQKGKILKQDTLNVIANEKQKVDYHNLLDKKEGDRATARQEWGVIETIKELKEGYLSQVIHKLTDLMIENNAIIVMEDLNFGFKRGRQKVEKQVYQKFEKMLIDKLNYLVDKNKKANELGGLLNAFQLANKFESFQKMGKQNGFIFYVPAWNTSKTDPATGFIDFLKPRYENLKQAKDFFEKFDSIRLNSKADYFEFAFDFKNFTGKADGGRTKWTVCTTNEDRYAWNRALNNNRGSQEKYDITAELKSLFDGKVDYKSGKDLKQQIASQELADFFRTLMKYLSVTLSLRHNNGEKGETEQDYILSPVADSMGKFFDSRKAGDDMPKNADANGAYHIALKGLWCLEQISKTDDLKKVKLAISNKEWLEFMQTLKG

SEQ ID NO: 94 - protein, 1390 aa, unidentified
  source 1..1390: note = Description of Unknown: Cas12 sequence
MIDNTKEKKEGSVFDGFTRKYQLSKTLRFELRPILNTPKMLDDEQVIKNDETRRKKYEAVKPWFDQLHREFIEDALKSFKFKNLAIYQDTFQTWQKDRKSKQKKDTLVKIEVGLREEIVRRFEEVANIWVRSEQYKLLGIKKEGLGMLFEAGVFRLLKERFKNEKDTTVDGNNIFDEWTRWTGYFKKFFETRKNFYKSDDTSTAIAYRVINQNLRRFCENIQIFEKISEKIEFSEVEKSFDISCAGIFSLAYYNACLLQGGIDTYNKIIGGEVDEKDKKIPGINELINKYRQDNSGEKIPFLKQLDKQIHSAKEAFIESIETNKELVGKLKTFYENAEVKIQSFRNLIADIVTDYSGYDIDKIYLTKEAVSHNASRWFASFESFERDLFAVVAEKQNKLVYELLRTHKNDSKISDKDGKFSFPDFIKCSHIKRALEKQEGRIWKGEYYEDIVDFEKIKDVFTQFLCVFKFELEQQFFRKTTSAQTGEQTKIGYEIFVTKINELITRENPVIDLEEKIAIKNFADATLLIYQIAKYFAVEKRRGWLDNYDLDDRFYKSSDIGYLNFYRDAFEQIVRPYNLFRNYLTKKPYNTNKWVLSFENPTLADGWDKNKEKTNAAVILRKDGRYYLGIIKEDCKSLFADRYSKEMSEGIESGSFQKMAYKFFPEASKMIPKCSTQTKNVKEHFRKSSSDYNLFHEKDYKISVAITKNIYELNNVFYRKDNIEESFVPKNDFEKKLGVKKFQRQYLEISRDNNGYKQALAQWIEFCIRFLKAYKSTTIFDYSRLREAKEYESLDAFYQDINALTYNISFVPISEQYIKEKNDNGELFLFEIYNKDWSLGPMDKNRKRTKNLHTLYFEQLFSKENEQENFLFQLNGEAELFFRPKTEEKRLGYKVWDAGEKKWVKAKEKEDGAVIDRKRYAKDIILFHCPITLNRVSESKTKREMDVEIREVLSSTPGVHIIGVDRGEKHLAYYSVIDQNGKIIETDTLNSIGKDGRGKPVEYASKLEKRAQEREASRRDWEEVEAIKDLKKGYISQVIRNLADLIIKHNAIIVFEDLNMRFKQIRGGIEKSAYQQLERALIDKLSFLVKKGEEDPKQTGHILRAYQLAAPVIAFKDMGKQTGLIFYTQAGYTSKTCPECGYRRNIKCLFENIEQAKTLIENLESINYNKKEDVFQISYSLEKLSSKDQKKEKKVSNELYAKTLKKDIFILTTKNALRYKWYDRYSEKAKVAKRGIDEYKGEVNESETKKGVVKEFNLTEYLKGLLKTYEIDYEHGGIREQILSVARGREFYKDFLYALFLLTETRHSISGRNTDYIQCPECEFDSRKGFKDIKEFNGDANGAYNIARKGIMILEKIKQFKKDNDGNLEKMGWGDLSISIEEWDKFTQKE

SEQ ID NO: 95 - protein, 1333 aa, unidentified
  source 1..1333: note = Description of Unknown: Cas12 sequence
MKNFQDFTNLYELSKTLRFELKPIGGTKKLIEEKNILKLDKKKRENYEKVKPYFNKIHQEFINFALRNPNFDFSQFEEKYLNWLKDKKNKDLLKEKESIDKIFLEKIGKLFENSVKDFLKENGFESIVKEEDQNLKFFRRKEIFEVLQEKYGSELETQMVNKDGEIKSIFNGWEKWLGYFDKFFNTRDNFYKTDGTSTAIATRIIKDNLKIFLENIVAFGKIKNKKIDFSEVEKNFSVSIDTFFEINNFNNCFLQDGIDFYNKVIGGETLENGEKLKGLNEIINKYRQDTGEKIPYFKKLQKQILSEKDGVFIDKIEDDGGFYEVLKNFYKNAAEKEGFLKNIFENFYTISDKNLEKIYFNKIAFNTISHKFGSALEFERILYEEMKKEKADGIKFEKKENKYKFPDFIQIIFIKRSLENYDSENLFWKERYYKSEENVDGFLEKNNNNLWGQFCKILNFEFLNILKRRIIDEAGEEYEVGFEISKNILGEKLENFELNQENKGIIKDFADYSLALYSFGKYFAVEKGRNWDLNIDISDDFYGGEDGYIEKFYNTGYDEIVKPYNLMRNYISKKPWEDNKKWKINFETSSLLSGWDKNLESNGSYIFQKGNKYYLGIINGSKPAKEILEKLYSGDGEKIKRFIYDFQKPDNKNTPRMFIRSKKDSFSPAVEKYNLPINDILEIYDNGLFKTENKGNPNYKESLRKLIDYFKLGFSRHESFKHFNFVWKDSKSYENIADFYRDVEKSCYKIDFEFLNFEELKKLTFEKHLYLFQIYNKDFELDESLQEKGYNFKGEGQKNIHTKYFEALFLEENISRKSGAVFKLSGGGEVFFRKKSIKAKKEKRNSVEVIKNKRYTECKYFLHFPIQVNFKEEISGNFNQEINKFLANNPDINVIGIDRGEKHLAYFSVINQKGEILESGSFNKIENYNKNGEKLLFPEREIKEIHKDGSLIDLELVETGRKVDYVDYKLLLEYKERKRLLQRQSWKEVEQIKDLKKGYISALVRKIADLIIKHNAIVIFEDLNFRFKQIRGGIEKSIYQQLEKALIDKLNFLVNKNEINLEKAGSILKAYQLTVPVDSLKEIGKQTGVIFYTEAAYTSKIDPITGWRPNLYLKKNNSKINKENILKFDNIVFNSKENRFEFTYDLKKFFGKDSKFPAKTVNTVCSCVERFKWNRNLNNNKGGYIHYENLTDGKLANKEQKEDEFSNFKELFEKYFIDINGNILEQIKNLDTKNNEKFFSSFIDLFTLVCQIRNTNQNAKGDENDFILSPVEPFFDSRKSQNFGKSLPKNGDENGAFNIARKGLIILNRISENPEKPDLLIFNADWDNFARNI

SEQ ID NO: 96 - protein, 1329 aa, unidentified
  source 1..1329: note = Description of Unknown: Cas12 sequence
MSNNFQEFTQKYALSKTLRFELKPVGKTKEILEKEMPMYQIINADKNIKAKYIQTKPFFDQLHRDFIKEAFENVELSGLSDFFENWKIYKQDKKANEKIYKKSAENLRKEVVSFLNAKGKDWAEKYHSSGLKKADIEILFEEGIFKVLEIRYGTDTNSFITNATTGEITSIFQGWKGFTGYFLKFWNTRENYYKTDGTSTAIATRIVDQNLPGYLENLEIFEKMKGKIDFESVRGDFSDFEKIGTVEYYSTCLLQEGIDGYNRIIGGYTYENGEKIKGINEIINLYRQTHKDEKVPFLKTLDKQIGSEKIAFMETIDTPEEFRKIFEEFVLKSSEKVVLLKQCLNHLFENELTDGVFLSKESLNTISHKWIDIGNMKLFHESLFTILKKEGAKYDSKEDEYKFPDFIRISDIKTALVKITTESFFWKNRYLYEKDENPTGFLTSDNSLWEGFIQIFSHEFSSLFERTEKDEEGKDIQWGYDISLLNIQKLLENNEYNPNDEKNKIIIKSFADDILRIYQMGKYFALEKKRQWNPDNLEIGEFYSHPEIGYDKFYFDSYKIIVQGYNDIRNYLTKNPWSEEKWKLNFENPTLANGWDKNKETDNSCIFLKRDNKFFLALMSRGNNQVFDERNIQKFAQNIEQGKYEKMVYKYMKDVALGIPKATTQLNAVQEHFFQSDKDYIITKGGSSIGEFIKPLRVTKRIFELNNRIYPKDNLGISFLRNQVNKKEQKNYIKIFQKEFITLGGDEVVYKKAVHDWIDFCKEYTKSYPSCAYFDYSGLKDTKEYSSIDEFYNDLDSFGYQISWQDISSSYIDELVESGKLYLFEIYNQDFSNGKTGAKNLHTLYFEHIFSKENQEVNFPLKLNGQAELFFRPKSIEAKGENRKFNREIIAKKRYTEDKIFFHVPLTLNRTEGDIYGFNTEINNFLAHNPDINIIGIDRGEKHLAYYSVIDQKGNIIESDSLNTVNEINYGEKLTDTAEKRKQARQDWQAVEGIKNLKKGYISAVVHKLTDLIIKYNAIVIFEDLNMRFKQIRGGIEKSVYQQLEKALIEKLNYLVEKGEINPEKAGHLLNAYQLTAPFETFKDMGKQTGIVFYTQAAYTSKIDPVTGWRPHLYLKYSSAEQVKKEIAKFSNIIWNNTEKRFDFMYDIRNFSTQKEYPKNNIWTVCSSVERYRWDKTLNQNKGDYVHYKSITPEFEKLFSDFQIDGTKNILEQINRMETKGNEKFFKSFIFFFGLICQIRNTNKADSDENKQDFILSPVVPFFDSRDSENTKNGLPRNGDENGAYNIARKGLIILQKINEFSDENGNCDKLGWKELSISQVDWDNYIKT

SEQ ID NO: 97 - protein, 841 aa, unidentified
  source 1..841: note = Description of Unknown: Cas12 sequence
MEYDEHISLIESEEKADEMKKRLDMYMNMYHWAKAFIVDEVLDRDEMFYSDIDDIYNILENIVPLYNRVRNYVTQKPYTSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTGENFDISFCRDLIDYFKNSIEKHAEWRKYEFKFSATDSYNDISEFYREVEMQGYRIDWTYISEADINKLDEEGKIYLFQIYNKDFAENSTGKENLHTMYFKNIFSEENLKNIVIKLNGQAELFYRKASVKNPVKHKKDSVLVNKTYKNQLDNGDVVRIPIPDDIYNEIYKMYNGYIKESDLSGAAKEYLDKVEVRTAQKEIVKDYRYTVDKYFIHTPITINYKVTARNNVNDMAVKYIAQNDDIHVIGIDRGERNLIYISVIDSHGNIVKQKSYNILNNYDYKKKLVEKEKTREYARKNWKSIGNIKELKEGYISGVVHEIAMLMVEYNAIIAMEDLNYGFKRGRFKVERQVYQKFESMLINKLNYFASKGKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMGKTQWTVYTNGERLQSEFNNARRTGKTKSINLTETIKLLLKDNEINYADGHDVRIDMEKMDEDKNSEFFAQLLSLYKLTVQMRNSYTEAEEQEKGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 98 - protein, 886 aa, unidentified
  source 1..886: note = Description of Unknown: Cas12 sequence
MFGNWGVIQNAVMQNIKRVAPARKHKESEEDYEKRIAGIFKKADSFSISYINDCLNEADPNNAYFVENYFATFGAVNTPTMQRENLFALVQNAYTEITALLHSDYPTEKNLAQDKANVAKIKALLDAIKSLQHFVKPLLGKGDESDKDERFYGELASLWAELDTMTPLYNMIRNYMTRKPYSQKKIKLNFENPQLLGGWDANKEKDYATIILRRNGLYYLAIMNKDSKKLLGKAMPSDGECYEKMVYKLLPGANKMLPKVFFAKSRMEDFKPSKELVEKYNNGTHKKGKNFNIQDCHNLIDYFKQSIDKHEDWSKFGFKFSDTSTYEDLSGFYREVEQQGYKLSFARVSVSYINQLVEEGKMYLFQIYNKDFSEYSKGTPNMHTLYWKALFDERNLADVVYKLNGQAEMFYRKKSIENTHPTHPANHPILNKNKDNNKKESLFEYDLIKDRRYTVDKFMFHVPITMNFKSSGSENINQDVKAYLCHADDMHIIGIDRGERHLLYLVVIDLQGNIKEQFSLNEIVNDYNGNTYHTNYHDLLDVREDERLKARQSWQTIENIKELKEGYLSQVIHKITQLMVKYHAIVVLEDLNMGFMRGRQKVEKQVYQKFEKMLIEKLNYLVDKKADASVSGGLLNAYQLTSKFDSFQKLGKQSGFLFYIPAWNTSKIDPVTGFVNLLDTRYQNVEKAKSFFSKFDAIRYNKDKEWFEFNLDYDKFGKKAEGTRTKWTLCTRGMRIDTFRNKEKNSQWDNQEVDLTAEMKSLLEHYYIDIHSNLKDAISAQTDKAFFTGLLHILKLTLQMRNSITGTETDYLVSPVVDENGIFYDSRSCGDELPENADANGAYNIARKGLMMIEQIKDAKDLDNLKFDISNKAWLNFAQQKPYKNG

SEQ ID NO: 99 - protein, 1228 aa, unidentified
  source 1..1228: note = Description of Unknown: Cas12 sequence
MSNLYSNLHNLYQVQKTLRFELKPQGKTKENMEKVGILKADEHRAEIYSKVKKYCDEYHKLFIDKSLSNIELNGIDRYYELYSINNRDDKQKEELDQLEASLRKQISDAFKKSAEYKGLFQKDIITSYLVTMYKENQEKMQDIGEFNRFTTYFTGYNKNRENMYSEEDKSTAISYRLINENLPTFIDNIKIYKKIVSLMPEDIEKIYKDLEEYIQVDSIDEIFNISYYNDVLTQRGIECYNILISGRTKNDGDKIKGLNEYINEFNQTHNEKIPKLQELYKQILSDAESASFKIDVIKNDKELMNLIEVYYANILPILNKIEDLFTRISNYNLELILVNNDGTLSTLSNMVFNEWSYIKGAISEKYDEEYSGKEKYGTEKYAQKKQEYLKKQKIYSLKFLNDCIGNNAICEYLKNYIIQNKNIETIKEDYNEVQNIKVEDDTKELIKDEKSIEKIKKFLDDVKSLQEFVKLVIPKDRTVEKDAKFYSELTPYYEKIKEIIPLYNKVRNYVTQKPYSTEKIKLNFECPTLLKGWDANKEEANLGVILLKEGKYYLGIINPYCKKIFEVDEKDSNEQNNYKKMEYKLLPGPNKMLPKVFFSNSRIEEFNPSKELQEKYNKGYHKKGKDFDINFCHELIDFYKQSVNKHEDWKKFNFKFKDTSEYNDISEFYREVEKQGYKIEYTEYSEKYINELVDRGELYLFQIYNKDFSEYSKGKENLHTLYWKAVFDPDNIMNPVYKLNGNAEVFYRKKSLEMKVTHPANQPIANKNISTIEAGRSTSTFKYDLIKDKRYTMDKFQFHVPITMNFKSERLFNINQIVNKYLKYNDDIHVIGIDRGERNLLYVCVIDKNEKIVYQKSLNEIVSEYNNNRYTTDYHGLLDRKEKEREIAREDWKNIENIKELKEGYMSQVIHILVELMKKYNAIIVIEDLNKGFKNSRIKVEKQVYQKFEKMFIDKLNYLVFKDEDKMNEGGVLNAYQLTNKFESFTKLGKQSGILYYIPAWCTSKIDPTTGFINRFYLKYENFDKSKEFVNRIDDIRYNEKENLFEFDIDYSKFTDRLNDTKNKWTLCSYGERILTQKNANGEWLDRRIQLSIEFKKLFEKYGINLNNIKDSILKLDKDNLEFYKGNGESLGFIQLFKLMVQMRNSLTGKEEDNLISPVKNQHGKFFNTSEKIEGLPIDADANGAYNIARKGFMLVEQMKNVEDEKLNKIKYNITEKEWLNYVQNRGM

SEQ ID NO: 100 - protein, 1142 aa, unidentified
  source 1..1142: note = Description of Unknown: Cas12 sequence
MARIFEDFKRLYPLSKTLRFEAKPIGATLDNIVKSGLLDEDEHRAESYVKVKKLIDEYHKVFIDRVLDNGCLPLKNEGHNNSLTEYYDSYVSKSQNEDAKKAFEENQQNLRSIIAKKLTEDKAYANLFGKNLIESYKDKTDKTKIIDSDLIQFINTAESTQLNSMSQDEAKELVKEFWGFTTYFVGFFDNCKNMYTAEEKSTGIAYRLINENLPKFIDNMEAFKKAIARPEIQANMEELYSNFSEYLNVESIQEMFLLDYYNMLLTQKQIDVYNAIIGGKTDDEHDVKIKGINEYINLYNQQHKDDKLPKLKALFKQILSDRNAISWLPEEFNSDQEVLNAIKDCYERLSENVLGDKVLKSLLGSLADYSLDGIFIRNDLQLTDISQKMFGNWGVIQNAIMQNIKRVAPARKHKESEEDYEKRIAGIFKKADSFSISYINDCLNEADPNNAYFVENYFATFGAVNTPTMQRENLFALVQNAYTEVASLLHSYYPAEKKLAQDKANVAKIKALLDAIKSLQHFVKPLLGKGDESDKDERFYGELASLWAELDTVTPLYNMIRNYITRKPYSQKKIKLNFENPQLLGGWDANKEKDYATIILRRNGLYYLAIMDKDSRKLLGKAMPSDGECYEKMVYKLLPGANKMLPKVFFAKSRMDDFKPSKELIEKYNNGTHKKGKNFDIQDCHNLIDYFKQSIDKHEDWSKFGFNFSDTSTYEDLSGFYREVEQQGYKLSFARVSVSYINQLVEEGKMYLFQIYNKDFSEYSKGTPNMHTLYWKALFDERNLADVVYKLNGQAEMFYRKKSIENTHPTHPANHPILNKNKDNKKKESLFEYDLIKDRRYTVDKFMFHVPITMNFKSVGSENINQGVKEYLHHADDMHIIGIDRGERHLLYLVVIDLQGNIKEQYSLNEIVNEYNGNTYHTNYHDLLDAREDERLKARQSWQTIENIKELKEGYLSQVIHKITQLMVKYHAIVVLEDLNMGFMRGRQKVEKQVYQKFEKMLIDKLNYLVDKKADASVSGGLLNAYQLTSKFDSFQKLGKQSGFLFYIPAWNTSKIDPVTGFVNLLDARYQNVEKAKAFFSKFDAIRYKRIRTGLSLISTMTSLVKKQKVQGLSGFYAPEECVLILSEIKKKTHNGIIRKLT

SEQ ID NO: 101 - protein, 1250 aa, unidentified
  source 1..1250: note = Description of Unknown: Cas12 sequence
MISLNYFQNQYAVAKTLCLELRPIEKTMEYIISSGILKEDEHRNESYKLVKKIIDDYHKAYIELSLSRFELKITSCSKNDALEDFYCQYLANSQEEKDKNIFKKTQDNLRKQIAKHLTQGEAYKRIDKKELIQEDLLEFVAADPDAANKKILINEFRDFTTYFTGFYENRKNMYSEEAQSTAIAYRIIHENLPKFIDNMGTFKQLMQSSITDILPQIFDNFKKDLEVSSIQEIFDLNYFNKVLTQKQIDIYNAIIGGKSLNENSRIQGLNEYINLYNQQHKENKLPLLKLLFKQILSDRNSLSWLPEAFETDKQVLHAVRKCYANLKESVLHEAGLVQLLSSLPSYDSTRIYIRNDQALTTISQKLFGDWGIIPHAIKERLKKDISAKRKETEEAYLERIEKAFKQADSYTIAYINDSLKEIGVDKKNIEDYFIHLGAICTEGQEQENILQRIASAYSQAQPLLEEKVPVHKNLMQDKDSVELIKSLLDELKNLQHFIKPLLGKGSESDKDERFYGEFVGLWNELDQITTLYNKVRNYVTRKPYSIEKFKINFQNATLLKGWDRNKERDNTSIILRKNGLYYLAIMRKEYNKVFEKYPAGTEENCYEKMEYKLLPGANKMLPKVFFSKSRINEFNPSPQLLQNYQMGTHKKGDQFKKEDCHALIDFFKTSIEKHEDWKNFNFQFSPTSVYEDMSGFYREVEQQGYKLVFRSIDAEYIDKLVEEGKIFLFQIYNKDFSPFSKGTPNLHTLYWKMLFDERNLNNVVYKLNGEAEIFFRKKSITYTHPTHPAEIPIKNKNVQNKKKESVFQYDLIKNHRFTIDSFQFHVPITMNFKNAGLSNLNEQVYTYLRENKDAHIIGIDRGERHLLYLVVIDRYGRIVKQFSLNEIVNEYHGNTYTTNYHDLLDKREEARQQARQSWQSIENIKELKEGYLSQVVHKIANLMVEYHAIVVLEDLNIGFMRGRQKVEKQVYQKFEKMLIDKLNYLVDKKKAPEADGGLLKAFQLTNQFESFQKLGKQSGFLFYVPAWNTSKIDPCTGFTNLLDTRYENIAKAQKFFRTFDAIRYNAVKDYFELELDYDKFHKRAEGTQTKWTLCTYGTRIKTFRNPENNNQWDNVEINLTEEFKKLFKQFGINLSGDLQQAICAQTEKSFFESLLRLLKLTLQMRNSITGTDVDYLLSPVQNAEGYFYDSRKGDKSLPANADANGAYNIARKGLWVIQQIKQTPQGQKAKLSISNKEWLKFAQEKPYLKD

SEQ ID NO: 102 - protein, 1193 aa, unidentified
  source 1..1193: note = Description of Unknown: Cas12 sequence
MKTFENFTNLYSLPRTLRFELKPLYKTKELIDSKQELFPKDKRIDEIYQNIIKPCLNELHSDFIEKSMENKDFQNIPDNILKIYSNEKNIDDFKNIEKDLIKQINWFLKSNKTFFAENYSDLLGKNSIDIIIKVFWEKIYKKDDSWKIFLYNDLLWKSYEELINIYFKWFSTYLSNFNKNRENLYDKKNEAKVWSVSGRTIWENFPRFLQNCINFRDKLEKLNLSIEQKDIFITNNFWKCISQKQIDYYNKIIWQINSKTNEFNQKNGLKWNKKLPKLLLLHKQILWKSENENILNFINNIIQTDFELEQEIKIINKDIFQRIDFIKKSIVSNIEDFELEKIFIKKNRLKDISSLLMDNYSVLEKLLPEFNEEWKIIKENELVNLSKIKKSFENIDLKDLKNIFKKEYFDESKDWFKLFLNWIYNHFSDLENNIKHTHKLVQDKLISWNFSENIQKSEKNINLRDEIFVSSKWLLKAYLDSILALDRFVHMFDYWEQKDFDSNFYNNIEEYSINFSPFKTYNAVRNYLTKKNYSTDKIKLNFDYPDFLGSNSLWKYAFIYKDSKWFYYLWVLDHSNSQSKYKPQILKNNTEFYQLEYKQIKFNTLAWKWYIRDFWVKYSEDENCIINLKTLIKKQYLERYPVLKEIVDFQTDDKKIFDAKVKTILEQAYSINFVNIDKNYILEENNNWNLHFFQIYNKDFSENKKINSMENLHTMYFKALFEKENFNWWACFKLNSQWAEIFFREKSINEKKVKDLKTRNENAIEKKRYTENKVFLHLPITLNFINKWYSKYSFWYINDSVKKYIKENKISIIWIDRWEKNLIYFSMINENLEIIELKSLNSLILKVSDLEEKEVNYFEKLSKKEWNRNKERKDWDEIETIKELKEWYISQIVDNLVKLIVKHNAIVVMEDLNSGFKRWRQKIEKQIYQKFELALAKKLNFTVDKNKKHDELWWIYKAYQLTPQIENFQDIYSQTWIIFYTQAAYTSVTCPNCSFRKNIYQKYENESKFKEFFKKYILEIKFEDNCFIIKYKIDEKIDKKKNKLKKLEFQVNTKNQIRLKFEKSLKWKWWETKEFNITEKFKEIFEKHKLDLWNLKEELLAWNWEIQLYKDFMFYFNLLLQLRNSKENDNWWYISCPSCWFHSGNWFQWFSYNWDANWAYNIARKWRIILDKIKKDEKNLWITNVEWDNYYQK

SEQ ID NO: 103 - protein, 1202 aa, unidentified
  source 1..1202: note = Description of Unknown: Cas12 sequence
MAVRSVKLKLLVPRDGSAESVRKRKALWATHQFVNDAAAAYAELLLEMRQEDVCRGTDDHGKDVIEPAAHWQAKLRARLAAKQLPPVAVAEALPLLKAFYGSRLIKSFVANDKGVAGTGNATDLNTWLSGLVDPASVAGEKTELRKQLLAELPLCEAADADFEGAARKMLAKSDAREALLEGPGTGVGWPAAYNANPTDSVWLDMLHKAAAKARLELADTTVSELKKLGVFPLLQAASSNRVFGSGVLNPFERMAAAQAAAALLPWETKRHEMRKRRDKFADQLNQWDTEFGASHATALAAIRAFEAEESERARRESLGNEGTGYRIGGRELRDAWTLLRDWLKGHSTATAAAREDKVRELQAKQGRSFGSHRLLSWLAKPAQQWLADHSAGDVVTRIAVRNARQRKLDTARTLPIWTGADAVKHPRFANFDPPNNTNQPGFDLRAGTQKGRLTLRLSLLTERADGLLLAQDHDFQLVPSRQMAEIVLHKDGKERALSWQSQDGIGRQVGDVGGSALLFSRDHAECLLERKQITRLERGAWPAALPVWFKLSLDIGAEHKALLKQRFKWGVWLNSALVTRNAKDAKGVPPPVGTRVLAVDLGLRSAATVSVWQVVDAATPVVAGKWRVPLSDTLSAVHERSAMLALPGEHVDAGVLAARRAANEKLAGLLAATSHLSTVFKLGRAEQGDRRRELLERLGEGDDRRARAAVATTAAERDGLRAVLGATQDAWAGAVAAVWRRLETDLAGAIAAYRKQQREDVQLRREARHGPGASQLPKQAAAERLLGGKSAWQIEYKERVRKLLTRWIMRQRPGDTAVRRLARKDLGKYCGGLLDHLTALKEDRAKTTADLIVQAARGRVRAHKDAHGRQQDRELWLAKYAPCDLIVMEDLGRYRFATDRPPSENRQLMQWTHREVFRLVQMQAEVEGIQVLETGAEFSSKFDARTWAPGVRCEPITKLWVERYRNGEMPWLADKADEWRREGIELAQLVPGQLLPTGSGEQFVAVSATGGLRVRHADLNAAQCIALRALTGHGTAFRLTARRLGDVFVSAKGLGKRPQGALWREFGSALPPAVVVLRPAGEVRYALRPFASARDAAAALGLQLGALRNVDATDAESDAEDGDLAELLAGADPDRATFFRDPSGDVHGGAWVQAKVFWAEVRRHVRLGLQAQGLLPAAARSSEPRQMQLPLAGALPGDDIPL

SEQ ID NO: 104 - protein, 669 aa, unidentified
  source 1..669: note = Description of Unknown: Cas12 sequence
MLDKFASLYPVTKTLRFRLLPQGRTEENMQVAKVLENDLERSEAAAVVKGLIKKYHLQFISDTLSGSTLSWQALTETLDKFKADHTATAELDSALAAYRCKLAELFTKSPKYKVMATPVSIIKEILKTETDPENIAALNKLNGYTYIIFDYVSTRMLTYSADAKATSLAYRLVDENYLRFYQDISAAAEISAVLEEAGFDNAEVEAFIRTDYNTCLTSEGIASFNAAAGSINQFVNVLLQQNPVLQSEPALRRHLQPLYKMLLDEAESKIIKFEDYGQLRDAVENFRRNFQDLPQSLIDIFAGRYDYSKIYVGYKYLNEASSQIAGGYNWKLLENALEDFYSKPYLVNGKLPVKYKTVVNKKMNQLAYSFTELQEALDAGDSGSSITDLFGKYAELHAAYAAADGNVFYKEYDRKSIASLKNYLDAVNAIARFIKIFAAPEVYVKDEGFYGIVDGAADKLRDFDLLYNMVRNYITKKPYKKSKVALTFNSSSFGRGWDENKIYDELTTIFTYNGKYYLGVINKNDKPDLAAAVSKDEGGYKRMVYKTFDIVKQLPRLSFTKAVKAHFAESDEDFIFDGPKFAKPLRVPKEIYLQSFTDNGDKLADSAKKYTKAYLDMSGDYKGYYEAIIKRIDYTKEFLSAYKSTSIYDLAFLKPAGKAAGSLCWTRHI

SEQ ID NO: 105 - protein, 1193 aa, unidentified; identical to SEQ ID NO: 102
  source 1..1193: note = Description of Unknown: Cas12 sequence

SEQ ID NO: 106 - protein, 1583 aa, unidentified
  source 1..1583: note = Description of Unknown: Cas12 sequence
MSFLVPHLPTVVSHRLGGYSSAMDQTPTRLTPAVKNTAAATPKPEVPLTQRAYTLRLRGANDGDQSWREAVWATHEAVNMGAKVFGDWLLTLRGGLDRELADAKVKAGNNNPDRNPTPEERRGRRVLLALSWLSVESAPKKGDAYEKFVIASGKKDSQSIRDEKVVRALREILAKRGVANQDLEGWIVDCEPSLSAAIRDDAVWVNRSAAFDAAQCRVGTSLTREEIWDLLKPFFGSCESYLASLTTDEDSDTEAAATDDKAKDLVQKAGQWLSSRFGTGKGADFAAMSKVYSETAFWAGRASPFRSGAEALRLIAESLKSFCPKSFDADGILGLISGPGYKSATRNIIKAWSKRAGPVTADDLANLSAVAAEDANKCSANTGSKGHRPWSDAILCEVENACGFTYLQPDGPALHSEFAVMLDHAARRVSIGHSWIKRAEAERDRFTKDALRIKEVPDPIRVCLDRFCADRAGTSGAIDGYRIRKRAVSAWKEVITRWGQAACKTAEDRVIAVRETQADPDIDKFGDIQLFEALAVDDAECVWRVNGEVTPQPLIDYAAATDAEAKQKRFKVPAYRHPDPLSHPVFCDFGNSRWKIRFAAHDAVTKLANARTSLDRREADLAKAKERLDKATAPEEQTEGKESLEEAERHLRDARDRVAWLSSMHAFSMRLWGAGRVGDGQRLFWSCKRLTDDMALRQNSGQAPTIAVTRADRLGRAAAGADTADSVDILGVFTEEHWNGRLQAPRAQLDVIAAHVAKNQWDAKAKKMRDRIRWLVSFSAKLQPVGPWIEYSATFPEAALAKPFVSRKGEYAVRHVSNDDRAGLGKLVLSRLPGLRVLSVDLGHRYAAACAVWETVSIAQTNAACDAAGHELPTESDLFLQLSTTDLTGKNRTTIYRRIGADMITDPKTGEKTPHPAPWARLERQFLVKLPGEDIPARKASPAEFDAIRRLEEAFGRTRTADDPLLVRVDELLAATVDSARLALRRHGDAARIAYAFKPSAEKLTPGGGREVMSPEARKAMILDALLLWHGLWHGDRWADVWASQQWDAYIKPELGMDLPPWSETSGEPRCQYRSKVEGLLKRVAESLAARDGAGLHLLWAEQWRTRNAKWLGNTGHLRTLRSLLLPRGLTTSTPAAWNVGGLSLTRIATLKSLYQLHKAYHMRPEPEDPRKNVPAKGEEELRDFGRGMLDVMERLREQRVKQLASRLAEAALGIGRMKASEGKRDRKRPRAQIDQPCHAVVIENLKNYRPEETRTRRENRQLMSWSSSKVKKYLSEACQLNGLHLREVQASYTSRQDSRTGAPGIRCADVPVQDFFTKPWWRRQVSIAVGRVNQGRGDARERFLADLDAKWSAAEKSERITAPPLRIPVNGGELFVSADLHSPAALGLQADLNAAANIGLKALLDPDWPGKWWFVPASLDADGWRVPAAKSCAGAEWVKNWKVGQLGDSYAPNGKPLQPTDDEGVKKAEDGVKLAKASLDDAEQALKAAKKTKRKAEIDVASARAQEAKKNVDDMKKALVAAKKGASAKEIINLWRDPVGVDPADFASGEPWRAYTVYKQRVEYHVINDVLRGRAGPRSDRP

SEQ ID NO: 107 - protein, 1254 aa, unidentified; identical to SEQ ID NO: 36
  source 1..1254: note = Description of Unknown: Cas12 sequence

SEQ ID NO: 108 - protein, 1468 aa, unidentified; identical to SEQ ID NO: 34
  source 1..1468: note = Description of Unknown: Cas12 sequence

SEQ ID NO: 109 - protein, 669 aa, unidentified; identical to SEQ ID NO: 104
  source 1..669: note = Description of Unknown: Cas12 sequence

SEQ ID NO: 110 - protein, 677 aa, unidentified
  source 1..677: note = Description of Unknown: Cas12 sequence
  VARIANT 19..339: note = MOD_RES - Any amino acid
MCIRDRDLAVAALNRGDAXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXGQLNAIRNFLASDGYGAHDLVPVNYNGSNDRGAEVQVGNQKNWTVTSSAPVVVQYNNADTTLTVQGHTDRLIVTGSGNDTITLKDSGDDKVLLGDGNNTVVAGSGADTIVGGAGNDVLIGTNGGYGTELLGGAGNDVLRNAGSHGIYMDGGAGNDTFYGGTGPDTMEGGDGNDLMYANGRGSSIDGGAGNDTIYGGPGGDTLTGGDGNDLLRSDSAFGTKGSGNLLVGGAGNDTLWAGAGYDTLKAGSGSDTLISGTGSSQMIGGSSGNTTFEVAYHTGNDTITGSGSGNTVYLDGRDFSDATISNHSGVTTVSFSDGQVLKISGVQDIVFSDHDYKV

SEQ ID NO: 111 - protein, 1254 aa, unidentified; identical to SEQ ID NO: 36
  source 1..1254: note = Description of Unknown: Cas12 sequence

SEQ ID NO: 112 - protein, 767 aa, unidentified
  source 1..767: note = Description of Unknown: Cas12 sequence
MAQASSTPAVSPRPRPRYREERTLVRKLLPRPGQSKQEFRENVKKLRKAFLQFNADVSGVCQWAIQFRPRYGKPAEPTETFWKFFLEPETSLPPNDSRSPEFRRLQAFEAAAGINGAAALDDPAFTNELRDSILAVASRPKTKEAQRLFSRLKDYQPAHRMILAKVAAEWIESRYRRAHQNWERNYEEWKKEKQEWEQNHPELTPEIREAFNQIFQQLEVKEKRVRICPAARLLQNKDNCQYAGKNKHSVLCNQFNEFKKNHLQGKAIKFFYKDAEKYLRCGLQSLKPNVQGPFREDWNKYLRYMNLKEETLRGKNGGRLPHCKNLGQECEFNPHTALCKQYQQQLSSRPDLVQHDELYRKWRREYWREPRKPVFRYPSVKRHSIAKIFGENYFQADFKNSVVGLRLDSMPAGQYLEFAFAPWPRNYRPQPGETEISSVHLHFVGTRPRIGFRFRVPHKRSRFDCTQEELDELRSRTFPRKAQDQKFLEAARKRLLETFPGNAEQELRLLAVDLGTDSARAAFFIGKTFQQAFPLKIVKIEKLYEQWPNQKQAGDRRDASSKQPRPGLSRDHVGRHLQKMRAQASEIAQKRQELTGTPAPETTTDQAAKKATLQPFDLRGLTVHTARMIRDWARLNARQIIQLAEENQVDLIVLESLRGFRPPGYENLDQEKKRRVAFFAHGRIRRKVTEKAVERGMRVVTVPYLASSKVCAECRKKQKDNKQWEKNKKRGLFKCEGCGSQAQVDENAARVLGRVFWGEIELPTAIP

SEQ ID NO: 113 - protein, 1210 aa, unidentified
  source 1..1210: note = Description of Unknown: Cas12 sequence
MNIIMENFEKFVNLYELSKTLRFELIPFSQTKVELEKDWIIEKDREIEEKYHIIKEKLDTLHIKFVWQALEWVDLSLLEEYAELYFACKKDTKNKKLKSKFEKLEKKIRQEITSFFDAEWNKWKEKYGFLKKWWTSFLTEKEILDVLIDIFPENKDDFEIFKWFFTYFSNFNESRKNFYKDEWKAGQIATRAIDENLTTFLENIIKYKNFKKENPDFFTENEEKVFELDFYNFCLTQKWIDNYNEIIWAKSLEEWKNTQGVNQRINLLKQKNEKSNKKNLSYPKFDILYKQILSEKSENDFIPNIENTEELFTVIQKSIKENDKKITEIDKLFKKFFLEENNIDIWKVYISKQAVNTISSKYFENWSSLWWYLWENSKKKYFSLWEIKEALEDIKEKNIFKGEYYNNKIAFENKSNFENFLAIFYYEFQTNLSLLNWNQNNLESLQEKEFKKEEKQVDIIKKYFDSVMDLYAMSKYFFVDLKQAKNFPKDIEFYNDFDLYFSDYEPWKVYNLVRNFLTKKEVKTDKFKLNFSNSQFLTGWDKDKEKERFWVILRKNEKYFLAILKKNNNKIFENYRENNPTDFYEKMEYKQLNNVYRQIPRLGFPLQKKLDSLKWKELEEYLEKYKNNFWYNKEIAFIKEEFDIFQKNKEKWEKFDREKLKKLIDYYKKVVLEKYSDLYDLKKLENTDYDELVNFYDDVEKSMYSLHFTKIETEFLENLEKNWEIYLFQIYNKDFSDYKKENTKENIHTKYFKHLFSEENLENLKIKLSGWAEIFFRDKTHNLKQKLDKNWKKMFYWENKDKKVLEHRRYAKDSYGFHISITLWANNWDMYKFNQFFNKNFTPKHIIGIDRWEKHLAYYSVIDLEGNLVETDTLNIVNGINYLEKLENIEKSRMQERKSWWEIENIKNLKDGYISAVVSKLTELIEKYQAIIVFEDLNLGFKRWREKIERQVYQKLELALAKKLNYLTFKNKKDCEIWGVLNGIQLVPRVKDYQDIANYKQSGIIFYTNPAYTSTTCPECGWRKTLKFPSKITKTSILEFFKEIQMSFYWEKFSFTYENILWKSETLFSNVKRTQWNNKSRKIEHKENITQELKSVFEKYNINLWENISEKLQQTDMLLEDLKVLFYNFKLINNIRNSDSKLEEDIISCPCCLFNSENWFKGAYFNGDANGAYNTARKGIIMLENIKVNPERSNLFVRNEEWDEFLREEF

SEQ ID NO: 114 - protein, 1187 aa, unidentified
  source 1..1187: note = Description of Unknown: Cas12 sequence
MSKLFENMTNLYSLNKTLRFELKPVGNTRELIESKDFFKNDEEKAENYQFMKEKMDKIHRNYIQKSLETIKMLPILDQTEQKRLKKEDIKNELKSLRSFISAAFVSVKDLLSNKIIEWLISEASIEEKEKIKKFDKFFGYFKTYVQNRGNLYKAEDKAGQIAFRLIDENLPRFFKAKQIIEEIIKKTPDFVVKERNEKQEETEKKITEYLSIFYLNSYCHYLSQSGIDLFNEIVGHINLSINLYKQKTGVKFSLIPLLYKLPLAPRKQISRLPKQIENPEELELIVKSVLDRIDQKINPFINNILQTVLEENSVYDLNWIYISIKTTEQWWSIGLSGRNSLRELGYNQSKNWEEPVLKAEKNKFPFISLGEIKSFLDAKQLAWDDIKSVFWEGKSQSLMNWSWSNIFFHLIGKEISSLIEKYLHSRKNYQISQSKENQKHVLDNSLSLWRLLGWFVLMHKKTHLTPEIKESFFYDWENGLDAIVFDENSDPIHTIYDKVRNCLTKKPYSNKDKIKVNFDCPYLLWGWDQNYDAFGWLIFHDWKKYFLWLIKGSWLNMEEKNKLYEWINPMNSITKIIYDYQKPDFKNVPRLFIRSKWDTFAPMVREYWLPVNDILYLYDNELYKPDKKNPWKHKSYLRRLIDYFKLWLSKHKSFKHYIFKRKESDQYENLAEFYSDVEFSCYAIKKEKVNFDQVKALCESERLYLFEIYNKDYNQFSKWKNKNLHTQYFEALFEERDNNLFMLSGGWSIFWRESNWKIVEKIRSYSPKYNIEIIDKRRYTKNKLMIHIPIVLNFCRNQEWRVNDMIKSLIQSQSNNFTILGIDRWEKHLLYYSLIRQDWTIIKTGSRNTITNKIKIVDYHKKLDDREKKRDEAQANWEQQEQIKDLKKWYISQVINEISKMIIEHNAIIVLEDLNGWFKRWRQKVEKSIYQQFELALAKKLNHLVFKNRWDTESWGTMKAYQLTPLVAQFQDLSFQTWVVFYTPAWYTSTTCPCCGWRKNIYFKYENEKQAKIELEKLNIVRENNYFSITYTAEWWNKKWKITWSVLNKTDRILTTKWQTRLQYDRASKNTKEYDITTDFNSVFTAKFLDYKIRLENAGSKQCRNLINSINLLLKIRNAKSGTDIDTIQCPACEFHSQWWFQWNEFNWDANWAYNIARKGKVIIDKIVKGEKNTTVSQIEFDNEIQK

SEQ ID NO: 115 - protein, 938 aa, unidentified
  source 1..938: note = Description of Unknown: Cas12 sequence
PAYRHPDPLLHPVFCDFGCSRWQICFDVRKNVRTTSPRRLCLTLFTGSGMELVPFSWQSKRLARDLALEQRHRNPEASEVTRADRLGRAAAGVSAGGAVSIKNIFNEENWNGRLQAPRSQLAAIAARVDKHGWDAKALRMLDRLRWLITFSPRLEPTGPWIEYALRIPDDAAAKPFLSRKNGFAVLHRSNDDRKGLAKLILSRLPGLRVLSVDLGHRYAAACAVWETLSADQVREACQAAGHDGPTECDLYLHLKKDGRTVIYRRIGADTLSDGTPHPAPWARLDRQFLIKLQGEEHKAREASNAEIWAVHQMEAALGRSVPLIDRLVASGWGQRTEGQRARLEALKQLGWRPVAEKLDSDDEPGVMEESPAIKPSLSIDELMSSAVRTMRLALKRHGDRARIAHYLITDEKTKPGNVKEQLDENGRIELLQDALVLWHDLFSSKGWQDNKAKELWYVHIATLPEYKAFQSSGEGHAGPERRGKPEEDREKLGVVAQALAPNVTLREALHNAWKKRWEEDDARWKALLRWCKDWILPRGEAANSPAIRKVGGLSLIRLATLTEFRRKVQVGFFARLRPDGKKAETGEKFGRKTLDALEELREQRVKQLASRIAEAALGIGKEHVFPTAKEPQKKCWCGSVHRKKDPKRPRERVHSPCHAVVIENLTHYRPEETRTRRENRQLMSWSSGKVKKYLAEACQLNGLHLREVNAAYTSRQDSRTGAPGIRCQDVPVKEFMRSPFWREQVAQAKKKQAEGKGDARERFLCDLDEQWKNRSEDEWKKAGPVRVPLRGGEIFVSADGTSPTAKGLHADLNAAANIGLCALTDPDWPGRWWYVPCDPASFKPIMDKVEGSAAVQPDQALRQPAQAQRGDAARDRKKRGKVGGRSREVVNLWRDVSSRPISPNDSWQEFTPYWNDVQARVVNILRQSAGLTRGQGEP

SEQ ID NO: 116 - protein, 1140 aa, unidentified
  source 1..1140: note = Description of Unknown: Cas12 sequence
MPLRSINIKMRLKRAAEGRALRQSLWLTHSVVNCAVAEIERVLLLCRGRGYWTGDDEPVSAETVQQQALAFARETQARNGQPGIGGDAEILAALRGLYEAIVPSVNRDEQGRPLEGNAQAAGGFAGPMMDAESEGFQSVFDKILEPLPSWVAKMTGGTRGWEQESVQWLKSPESERLQHASGSPPAWVRRLRTGEPWQEAFVQDQENKRKEVKGVPSLIQRLKKELGLLPLMRPPITLQFEDKRSGLTPWDRLTLRLAVAHLLSWESWNHRAADEHGRVTERLARLETEAAPLASLIEGLREYEKIRHEELKRVAQANDENPFRIGARGVRGWDRVREAWLGATDDTKEGRFTSVAALQTKLGGRFGDPDLFRWLAEEGREHLWGEHDPLPILARLNALSRLLRRKKDHAIYTAPDARLHPRWTAYEAPGGGNLRNYSFEISGNDLALRLPLLRRVESGLEEDSQRAEIALAPSGQFQSASWKGDGKPNRHLTYYSAHEQFSAELGGAEILYRRRHLENRKVKELEQGDIGPVWLKLVLDVQPNAPEGWFTPRGRVVTPPTVHHFNTALVNRSKHAPDLVPGLRVLAVDLGVRTFAACSVFELVQGEPGNGMAFLADQERDLWARHERSFLLPLPGEAVDSQLLAARRAAYNQLGLLRRDLGRLKGILRLSVKETAENRCGSLEELLASLEDEWNRGNAPAIDVAALLSAKGCLEMPQPAWEAAIAGTYQAAERELGNRVRDWRRTTRPRTTGEDDRRQRRGYSGGKSAWAVEYLDQVRRLLQGWSLHGRSYAQIRRLDREKMGTFAAGLLDHINALKQDRVKTGSDLIVQAARGYLPTGKKGWIKQYEPCRLILFEDLARYRFRTDRPRRENARLMRWNHRQILTETELQAEIFGLLIGTTGAGFSSRFHARSGAPGCRTRLLTAEDLRSPTLAKQLELLAETQGIDPKCFRPGMQIPWDSGADFVTLDSEGKLVQIHADVNAAQNLQRRFWTRFRDAYRISAVEIKQDGRTSWYPDREGVRLRGALSTIVGGDGYARLVAADDDDGFVLEKVTRPHWRRAAGAQAESGDDVGLDEVALEMAEAMDTDLERGEGRLVFFRDPSGSVIRADRWYQAKAFWGQVRSRVTRALGLRRPGASS

SEQ ID NO: 117 - protein, 669 aa, unidentified; identical to SEQ ID NO: 104
  source 1..669: note = Description of Unknown: Cas12 sequence

SEQ ID NO: 118 - protein, 1236 aa, unidentified
  source 1..1236: note = Description of Unknown: Cas12 sequence
MSRSIFSPFTNLYPIQKTLRRELKPLNENFQHDPALSALRNSEIPQRDEQREKDYQAIKPLLDEFHNQFITESLHSLEPQDRSDFILFYQTYQKKKKNKAEISEKELKSLDEEFESRTKALRNAIGTSFSVTAELRKSNPDYVSEKGKPFLTQKSYKILTEAGVLWLLEKKYTSDPEKLALIRRFGNFFTYFTGFNQNRENYYATDEKSTAVAYRAINENLLTFANNCELFEKLSVLSLSELEKKTFNPDSYSEYLTQSGIVFYNEMLANIRSKANLYTQEHKAKLPQPKLLYKQIWSPRGDTIPFDLIASEAEFQETLHTMIRETDQRIPEFNKLLEQIFEEKVDLSQIFFSKTSLNIISNRYFSSWHTLLEKGVELKLFKFKKNDEESFKLPAYLSLAELKELLESAPFQMAEKADADEEKHHQASLFKLQRENLHLEKSHSNWELLLKSMKSDFESFWTWEGEFWSYTLAKKALQSLSALESTNQEHKNLIKMLLDNALYAYRMLKWFKVDTSKLGFVPEGEFYPSLDQLLQDYPLPKWYDMIRNYLTRKTYSQAKLKLNFDCSTLLNGRDKNKEIQNLSVILRKDGKFYLAIMKKDQNKFFENSALYEGNLGTMEKMDYKLLPWANKMLPKCLMPGSDKKKYGASDQVLELYAKGSFKKSEKSFNLADLHTLIDFYKLALPKYEDWKVFNFQFQATENYQDISQFYREVEQQGYLLNWRKVNEKLIKQGIKDWSLFLFQISSKDFEGKSKTPDLQTLYWQQLFEFSTNVKLNGEAEIFFRPWSMKKEKKKLKVDNYDVFKHKRYTEDKILFHVPITLWFGNNEVSPSAPSKFNQKLNQELIIPHFDDLHVIGVDRWEKHLAFYSVVSVKTGKIVKQGTLNLLNGTDYEAKLSQKAENRLHARQNRDTIEKIADLKNGYISQVVNKLVELVLEYNAVIVFEDLNAGFKRGRQKIEQSVYQKLELALAKKLNFIVKKEKAVGEPWSVTSAYQLAPQINTFWDIKWKQRGIMLYTRANYTSVTDPLTGWRKQYYFKKGSSEEMKAQFFKSFKNLTRDAGQEAYIFDDGTWLLYSNVERRRGKRGDHRERTQIKYDPSLELDTLFSKYQIEKSDSLFDQLKNRELPQTFWTSFFRIIDLIMQIRNTDDEGRDIILSPIGNPQERFDSRKRYNQLPRDEKGGIIEESAFEYPTSWDANGAYNIARKGVMMLERIKENPEKPDLLIRDAEWDKKITRK

SEQ ID NO: 119 - protein, 1333 aa, unidentified; identical to SEQ ID NO: 56
  source 1..1333: note = Description of Unknown: Cas12 sequence

SEQ ID NO: 120 - protein, 926 aa, unidentified
  source 1..926: note = Description of Unknown: Cas12 sequence
RPGASAALSLTPLKTLFSENFSSFMCGNWQIINDSLKTYYNENIKSKGKAKEEKVKKAIKAIEYKSLADINQLVERYNNDELNRKAEEYISAINEKIKDLDVNEIEYDEKINLIENETKSEEIKSKLDSIMEIMHWTKMFIIEEEIEKDVNFYNEIEEIYDELQPLVTIYNRIRNYVTQKPYSEEKIKLNFGIPTLANGWSKTKEYDNNAIIMIRDGKYYLGIFNAKNKPDKKIMEGHQSEENGDYKKMIYRLLPGPNKMLPKVFMSKTGIAEYKPSQYILECYEQNKHIKSDKNFDIKFCRDLIDFFKTSINRHPEWSKFNFKFSETSEYEDISTFYREVEKQGYKIEWTYISEKEIKELDENGQLYLFQIYNKDFSEKSKGKENLHTMYLKNLFSEENLKNIVLKLNGEAEVFFRKSSIKKPIIHKKGSVLVNKTYNENGERKSIPEEQYTEIYKYLNSIGTNELSEKSKKLMEEGKVEYYKANYDIVKDYRYSVDKFFIHLPMTINFKAAGFSPINNIALKNIALKDDMHIIGIDRGERNLIYVSVIDTKGNIVEQRNFNIVNGIDYKEKLKQKELDRDNARKNWKEIGKIKDLKEGYLSLVVHEIAKLVVKYNAIITMEDLNQGFKRGRFKVERQVYQKFETMLINKLNYLVDKDLAVDQEGGLLRGYQLTYIPESLKVLGRQCGYIFYVPAAYTSKIDPTTGFVAIFNYKGMTDKDFVTSFDSIKYDDERGLFAFEFDYENFVTHKVEMARNKWTVYTYGERIKRKFKNGSWDTAEKVDLTYQMRSILEKYEIEYNKGQDILEQIEELDEKAQNGICKEIKYLVKDIVQMRNSLPDNAAEDYDAIISPVINNNGEFFDSTRGDEDKPLDADANGAYCIALKGLYEVMQIKKNWNEETEFPRKELKIRHQDWLDFIQNKRYL

SEQ ID NO: 121 - protein, 1466 aa, unidentified
  source 1..1466: note = Description of Unknown: Cas12 sequence
MQCGSLRHNSKSLDSALAYPARLTTGGNEGKTSEAHIENDSLPEITRRPSRLARDFAPSNPFPFMKRIYQGQITGMQFHGAAGEQAVPANQNWEQALWDHHGLFHDAVNYYLVCLLALARPGNPVYAIREKLDAHNGAEPDELMVWRTFRRRGCNRPGLRDSVAKYLTPGNDEPTTEECFAAVLAGNPLGATEEGRAILNEGLAQLLGKCTGESGCRNSAKEYLPRFTKPDFKGNFEEDTARLNRAEASKRLPFYLHDPATTPNSPALDEFGVLSIALPNPKRAELTGAEARDKLLEFSREWSARLPDGSADWRRLEAKITALGDSLAIPGYTSGSAKGENRFQLYAMFLFCFVEKSDFTLGLLRQTTRRPVEGEEPPPRSNHSATAGDPIRIARGGRGYVFRAFTSLKQWGGDTGGDLKWPKFDMAAFCEALKALHQVEAKAKQRAEERSKKQALLDYQRGRIRRFKPTANSEDATPPPVLAGDPRISRLEQLLNTDLQDEYEMSEGVAVAYGLHPRTIRGFREIRKRWISAVGNAPFSEAARATLIACVRSFQSENPGTVGSARLFEALAEESNWIIWREPSAAEQQSWRENADLPESAEFALDPLQALTDERELKDEIERLSGPIRFTPADAEHSRRQFYFSDVSQIDKRNRFRPRLNEVEVELAVRPNGHWTNVWVTLQYSAPRLLRDQLPTADEAGGGWQQAMMAALNLRAPLKKGGEAVSFADCAALSLMPDISPDGEKRLLLNFPVELDGEAIANQLGRSKRWETLQFGGADDESYWLRWPKTWIDETKVRRKAAPPKWWLSKEPFSVLGVDLGQRDAAACALVQVSPGNCPTGVCRHVGSADGVDWWATVRSMNMLRLPGENAKVMRDGRFQEELSGSRGRSASIDELKEAGDICARLGFVADTILGANGRALSFPELNDRLLFSLRSAQSRLARLQSWSCVAHADVPPARREGILRDISEAKDDPLGLKPLATAGNLEAIASTLREVILQERTSISAQLVRVADRILPLRGRRWEWVARPESPSNHMLRATAPDTDPRRKLVAGQRGLSLARVEQLESLRQRCQSLNRALMQVPGTPSKLGRRSRGVELPDPCPDLLDRLDALKEQRVNQTAHLILAQALGVRLRIHQVSGPQRTRSDSHGEYERIPGREPVDFLVLENLDRYLASQGRSRSENSRLMKWCHRAILLKLKQLCEPYGLRVLETPAAYSSKFSSRDGTAGFRAVEVTPDDLGSHRWRKHSERLADPGASLSRDEREESTRLMAFAERLKALNQDLIARQEAARSSNQPFRPKWRTLLAPQQLGPIFVPAVGKPLQADINAAINIALRAIASPDVDDIHLRIRAARSGDKFVVRAENTRERARWGSAEAEIGLPTGAGAKEIEERRSLLTEARVNFFFDPASVAAFDHGKVRDAKLAVTSGRGLWGTLRREEWAIVGKINNDRLAAHGLGRPFAERVDL

SEQ ID NO: 122 - protein, 1464 aa, unidentified
  source 1..1464: note = Description of Unknown: Cas12 sequence
MSQRDPLPSPTPRAYTLRLSTPDADNGAWRERLWKTHEVVNSGAEAFGDWLLSLRGGLDHRLVAVKVRVGRGASKTEREPTEDERRERRIVLALCWLSVESKKGAPQGRVVEDPVDALKRILGQRGLSEADAEQWVFDCRDSLSATIRDDATWVDRSAAFDETVQRLGSSLTREHAEQVIIEFFGDIHDYLALPKNMEEDGVTSPRGGSGKEFRTQARSWLSENWGAGQKGDKAQIVDALRKCSRCIAQEKPSTGSDLLRVLVRSLGGSPEETLRFDELRKLVGWRTGRRSTGAFALQRTVDMDRLSESDLESLEAKLTVEIDAKSPEASRSVPQWVESMRTAVEREVGMPFRSTRDLIGEFGVMLDHAARRVSATHSWIKRAEAERRKFEGDAAKLAQIPEEARQWLDQFCEDRSQELNALEPYRIRRRAVEGWESVVRTWAKADCKAEGDRVKAVRALQPEIEKFGDAALFEALAAEDALCVWLLDGKATPRPLLDYSAATDAQYRKRRFKVPAYRHPDALLHPVFCDFGESRWTIEFSAHRALGRRRKAQQLVDNKTVALQKAQERLRKAKAESSRSRAEEKVRAAEHALEAARKKLAFLQYRRALAVCLWTGKGVESTSLRWHSKRFAKDFAIGSSPEDGASTGVPVARADRLGRASANVPRGASVTISGVFEQKEWNGRLQAPRDALNAIAKVRDDETLPAADRSRRVQQMLSRLPWFVTFSARLVPQGPWLDYASEHSALGLRVDPKYWPHAEENRQRKGMARLILSRLPSLRVLSVDLGHRYAAACAVWETLSAQEMQRICREMGSAPPSPDDLYLHLRRADGNGKQRTTVYRRIGPDTLPDGGQHPAPWARLDRQFLIKLQGEDESARKASDSEIAVVKKFEADLGRPAMQRRSLRVDDLMSAAVRTARLALRRHGDRARIAFNLIADRRFRPGGAEEPLTDETRVDLLADTLATWHGLFSGGQCHDERAEEQWNEHIAPLLASSGASLSVPSDGDAVAATPARRRRKEVRGKLVPAAMELARRDLSQLSVLWVARWHQDDESWRARLRWLRDWILPRGAKADSGAIRHVGGLSVTRLATIRSLWQLQKAYRTQPEPEDPRKNVPKKGDTSLDDFGRTILDDLEHLRENRVKQLASRICEAALGVGVEQPNGGAKDPKRPQERRFSPCQAVVIENLTRYRPEETRTRRENRQLLNWSAGKVKQYLSEACELHGLHLREVSAAYTSRQDSRTGAPGIRCQDIPVVDFVRENGPCWGRLRSAQQSSGGTAEDQLLLALYERWSEKERTWRDQSGNVWAMDGEGRWVARNGTQLPQGSGLTPHPIRVPQRGGDIFVSAAPRSPAANGIQADLNAAANIGLWALLDPDWEGRWWRLPCSAMDLKPVKGSVNGSAVIHLDAPLKAAATKGAAKKDVVNLWRDISVRSLTEGVWRGYDAYWNLARYRVVQTLRPQVGIGSDE

SEQ ID NO: 123 - protein, 1247 aa, unidentified
  source 1..1247: note = Description of Unknown: Cas12 sequence
MFKGDAFTGLYEVQKTLRFELVPIGLTQSYLENDWVIQKDKEVEENYGKIKAYFDLIHKEFVRQSLENAWLCQLDDFYEKYIELHNSLETRKDKNLAKQFEKVMKSLKKEFVSFFDAKWNEWKQKFSFLKKWWIDVLNEKEVLDLMAEFYPDEKELFDKFDKFFTYFSNFKESRKNFYADDGRAWAIATRAIDENLITFIKNIEDFKKLNSSFREFVNDNFSEEDKQIFEIDFYNNCLLQPWIDKYNKIVWWYSLENWEKVQWLNEKINNFKQNQNKSNSKDLKFPRMKLLYKQILGDKEKKVYIDEIRDDKNLIDLIDNSKRRNQIKIDNANDIINDFINNNAKFELDKIYLTRQSINTISSKYFSSWDYIRWYFWTGELQEFVSFYDLKETFWKIEYETLENIFKDCYVKGINTESQNNIVFETQGIYENFLNIFKFEFNQNISQISLLEWELDKIQNEDIKKNEKQVEVIKNYFDSVMSVYKMTKYFSLEKWKKRVELDTDNNFYNDFNEYLEGFEIWKDYNLVRNYITKKQVNTDKIKLNFDNSQFLTWWDKDKENERLGIILRREWKYYLWILKKWNTLNFGDYLQKEWEIFYEKMNYKQLNNVYRQLPRLLFPLTKKLNELKWDELKKYLSKYIQNFWYNEEIAQIKIEFDIFQESKEKWEKFDIDKLRKLIEYYKKWVLALYSDLYDLEFIKYKNYDDLSIFYSDVEKKMYNLNFTKIDKSLIDGKVKSWELYLFQIYNKDFSESKKEWSTENIHTKYFKLLFNEKNLQNLVVKLSWWADIFFRDKTENLKFKKDKNGQEILDHRRFSQDKIMFHISITLNANCWDKYWFNQYVNEYMNKERDIKIIWIDRWEKHLAYYCVIDKSWKIFNNEIWTLNELNWVNYLEKLEKIESSRKDSRISWWEIENIKELKNGYISQVINKLTELIVKYNAIIVFEDLNIWFKRWRQKIEKQIYQKLELALAKKLNYLTQKDKKDDEILWNLKALQLVPKVNDYQDIWNYKQSWIMFYVRANYTSVTCPNCWLRKNLYISNSATKENQKKSLNSIAIKYNDWKFSFSYEIDDKSWKQKQSLNKKKFIVYSDIERFVYSPLEKLTKVIDVNKKLLELFRDFNLSLDINKQIQEKDLDSVFFKSLTHLFNLILQLRNSDSKDNKDYISCPSCYYHSNNWLQWFEFNWDANWAYNIARKGIILLDRIRKNQEKPDLYVSDIDWDNFVQSNQFPNTIIPIQNIEKQVPLNIKI

SEQ ID NO: 124 - protein, 1250 aa, unidentified
  source 1..1250: note = Description of Unknown: Cas12 sequence
MLSFNWVLFFFFSKMSRSIFSPFTNLYPIQKTLRRELKPLNENFQHDPALSALRNSEIPQRDEQREKDYQAIKPLLDEFHNQFITESLHSLEPQDRSDFILFYQTYQKKKKNKAEISEKELKSLDEEFESRTKALRNAIGTSFSVTAELRKSNPDYVSEKGKPFLTQKSYKILTEAGVLWLLEKKYTSDPEKLALIRRFGNFFTYFTGFNQNRENYYATDEKSTAVAYRAINENLLTFANNCELFEKLSVLSLSELEKKTFNPDSYSEYLTQSGIVFYNEMLANIRSKANLYTQEHKAKLPQPKLLYKQIWSPRGDTIPFDLIASEAEFQETLHTMIRETDQRIPEFNKLLEQIFEEKVDLSQIFFSKTSLNIISNRYFSSWHTLLEKGVELKLFKFKKNDEESFKLPAYLSLAELKELLESAPFQMAEKADADEEKHHQASLFKLQRENLHLEKSHSNWELLLKSMKSDFESFWTWEGEFWSYTLAKKALQSLSALESTNQEHKNLIKMLLDNALYAYRMLKWFKVDTSKLGFVPEGEFYPSLDQLLQDYPLPKWYDMIRNYLTRKTYSQAKLKLNFDCSTLLNGRDKNKEIQNLSVILRKDGKFYLAIMKKDQNKFFENSALYEGNLGTMEKMDYKLLPWANKMLPKCLMPGSDKKKYGASDQVLELYAKGSFKKSEKSFNLADLHTLIDFYKLALPKYEDWKVFNFQFQATENYQDISQFYREVEQQGYLLNWRKVNEKLIKQGIKDWSLFLFQISSKDFEGKSKTPDLQTLYWQQLFEFSTNVKLNGEAEIFFRPWSMKKEKKKLKVDNYDVFKHKRYTEDKILFHVPITLWFGNNEVSPSAPSKFNQKLNQELIIPHFDDLHVIGVDRWEKHLAFYSVVSVKTGKIVKQGTLNLLNGTDYEAKLSQKAENRLYARQNRDTIEKIADLKNGYISQVVNKLVELVLEYNAVIVFEDLNAGFKRGRQKIEQSVYQKLELALAKKLNFIVKKEKAVGEPWSVTSAYQLAPQINTFWDIKWKQRGIMLYTRANYTSVTDPLTGWRKQYYFKKGSSEEMKAQFFKSFKNLTRDAGQEAYIFDDGTWLLYSNVERRRGKRGDHRERTQIKYDPSLELDTLFSKYQIEKSDSLFDQLKNRELPQTFWTSFFRIIDLIMQIRNTDDEGRDIILSPIGNPQERFDSRKRYNQLPRDEKGGIIEESAFEYPTSWDANGAYNIARKGVMMLERIKENPEKPDLLIRDAEWDKKITRK

SEQ ID NO: 125 - protein, 669 aa, unidentified; identical to SEQ ID NO: 104
  source 1..669: note = Description of Unknown: Cas12 sequence

SEQ ID NO: 126 - protein, 1367 aa, unidentified
  source 1..1367: note = Description of Unknown: Cas12 sequence
MNRIYQGRVTKVEILNGKNADGQPQELPNWQTALWQHHELFQDAVNYYLFCLAALASSASSPMGKLRNQLGSVWEPFGRTGRRFKGLRDSVGPYLLPDRPTPSIEEAFAAALAGNNSQPELLQLAVDALVEDLGGDAAIQQEGRGYLPRLCSPAYNGQFPRGANSLQKEAAKIRLPTLLHNVATSRNLDALAAELEFGFFANPVPNAGPIAGQEARNKLLEALAWLKAKDTRLTHSANELEKRIGVLPDSVSFPTYSGGSINKEALKHRFFAYLLFQHVERSLVTFEILRDSYPTPKPSARKSSKATPDTMAQLTQFGDDPVKLARGTRGYVFRAFTSLPCWGAKAPSDILWSEFDIAAFKEALKTANQFRLKTKERLDKADELAGELAWMNGEKSKFKPSEHSEQEPPAVLKGDPRFEVLKQLFEVELAEEHYLAEGESVIYGLHPRTLRCYRELVERWNKTVQPGEVFTEATSKKLVASTDKFQAENKERIGSVTLFKKLLERDYWCLWQTPDVQTVAARQKAGFSSNIIEDYQRYLELQTDIARLKEPIRFTPADAEQSRRLFMFSDLAGKSKHKHLPNATPYGFAVDVALAASEGGVWKETRVRLHYSAPRLRRDGLRKGVGEDLKRTAWLQPMVAALNLSEPAPQDFSKCAVSLMPDKSWKGLRHLLNFPVTLDPTVVQKAIGSQARWANQFVAFGKGATEQKFFLRWPEEVSASQKKAGPWWDALRAFSCLSVDLGQRDAGAFALLDVRANADWGKKPSRFIGEADGRNWRAALAAAGLLRLPGEDALVWRDGKWQEELYGEKGRLATKEEWLETNAIFSALEQNSEEWIGADPKRRSFPELNSKLLVVARRGQSWLARLHRWLWMLGDENKRERALRELLEQERQEAWRKRAEGKDLLALKQSLTAEIQRLNGLLPEQLVFLANRCLPLRNRKWAWNQHPDKAFAEKGCHLLEMVEAPSHLPLLAGQRGISFERIGQLEELRRRFQSLNRVLQRELGAPPKSGRELRDDLVPDCCPDILAKLDRVKEQRVNQTAHLIVAQVLGVQLRKHQTPESERTERDLHGEYEIMPGRKPVDFIVLEDLSKYLSSQARGRGENVRLMKWCHRQVTAKVKELCEPFGLPVLETPAAYSSKFCSRTGVAGFRGVEVTLKDRQSFPWSKRLEEDSAEVAELFGWLETASAGHKTKQPRCLLAPMSLGPLFIPVTSQAPIMQADINAAINLGLRAMAAPSVHEIHVRIRSEAKDGQFRVRAQSKREKARWGNEPPPIKMLVETQRVALAREGGHPNFFVDPLKAATFDRAEVEGLGLPVASGRGLWKSVRDAEWKRCREINLERMRRWGFIMEAPRTLQVSRKEDEDDLTF

SEQ ID NO: 127 - protein, 1202 aa, unidentified; identical to SEQ ID NO: 103
  source 1..1202: note = Description of Unknown: Cas12 sequence

SEQ ID NO: 128 - protein, 1352 aa, unidentified
  source 1..1352: note = Description of Unknown: Cas12 sequence
MSTQKNPFNQFTNLYELQKTLRFELRPVPETKKLLEKGEGKNLIQMDLEIDRLYEKEMKPMFNILHEKFINESLGLVKLDCKKLKKLENLLAEADKLRKQIKEGRKNKNNISEVEKRLKIIIGDNSQGKNKNGEIAVLQDELRVLIVKAFNLTADKWKKELNNKETLLPEKKGKRKIKIKKSGPKILQEENVLAILAYFNPDKADIIKKFAGFFTYFSGFNQNRANYYTVKALATGVANRAINRNFLIFLANRKDFARFKERLPRLAEFDNYFELENYEKYLSQTGIEEYNDQIGKIKQIVNLEHNQQQKDNKFQLKGLATLEKQIGCRTKKQREEGGDKSAPKFLEKVGLGFQVSQDDDGEYLIWECLNYINKELAGKLKSIKDNYQKFFADWRTGAYDLEKIWFRKEALNTISGRWFGGNNWFIIGKALALTGVGKFDKRENTYKIPEFVSLAEIKTAFEMLENGVNYDFKKSKKKKEGDDTDVVKYSADNLFKEEYKKKGLIKNSLFETMLAVWQSEIKRKFEQIFDGYKLEKDDVFGRKKGEWVEPFIENFQKVSQEKFDRGVKDENGRSIHTEVVKNLIEEGYLRLFQLTKYHNLDKKGERDPRPFDGNFYATLDEFWKDNIVVVYHKALQSTLTKKPYSEDKIKLNFENGSLLGGFSDGQERSKAGVVLKNKNKFYLGILIDRGFFRTDKANPVYDNAQNNEWERLILTNLKFQTLAGKGFLGKHGVSYGEMGKDNPMMAVEYLQKFIKLKYLDKYPALNEVAHKKYTIKKEFDADVKNALKDCFTMNFKPVDFGMIRQGLTESLFYLFEIVNKDISSQAKNGKNVHTLYWEALFGDQNLKKPILALNGGAEIFYRESQREKLEKKLDKSGKEVLDHKRYGQDKYFLHASITINYGQPKNIKFKEVINEKISQNADRVNIIGIDRGEKHLLYYSVVSPEGVLLEQGSFNQIETKNKVDIKAVKAEYGERGELKKVELVPTGKKVKYVDYQILLDYYEKKRNLARRDWQTIGKIKDLKDGYLSQTVHRIYQLILKYNAVVAMEDLNVEFKAKRAAKVEKSVYKNFEMALAKKLNHLILKDRRADEIGGALRAYQLTPAIPANDVGKFDKAKQWGIMFYVRANYTSTTDPLTGWRKHKYISNSEKIDNIQKFFSPGDGIQIDYDTEKQCFKFSYDHELEGGAKKHWELFACDGLERFYWDNRERQIKKYNLYEEFEKLLGGLRKEENINIQIDGVSEFRWKDLVFFWNLLNQIRNTDRSAQGDENDFLQSPVWSEKYNCFYDSRKAPNNMPNNGDANGAFNIARKGQLILERIKKCSDIPKFGNDNNGKNPENNYFISDADWDKFAQK

SEQ ID NO: 129 - protein, 1129 aa, unidentified; identical to SEQ ID NO: 3
  source 1..1129: note = Description of Unknown: Cas12 sequence

SEQ ID NO: 130 - protein, 1352 aa, unidentified; identical to SEQ ID NO: 128
  source 1..1352: note = Description of Unknown: Cas12 sequence

SEQ ID NO: 131 - protein, 1108 aa, unidentified
  source 1..1108: note = Description of Unknown: Cas12 sequence
MAIRSIKLKLKTHTGPEAQNLRKGIWRTHRLLNEGVAYYMKMLLLFRQESTGERPKEELQEELICHIREQQQRNQADKNTQALPLDKALEALRQLYELLVPSSVGQSGDAQIISRKFLSPLVDPNSEGGKGTSKAGAKPTWQKKKEANDPTWEQDYEKWKKRREEDPTASVITTLEEYGIRPIFPLYTNTVTDIAWLPLQSNQFVRTWDRDMLQQAIERLLSWESWNKRVQEEYAKLKEKMAQLNEQLEGGQEWISLLEQYEENRERELRENMTAANDKYRITKRQMKGWNELYELWSTFPASASHEQYKEALKRVQQRLRGRFGDAHFFQYLMEEKNRLIWKGNPQRIHYFVARNELTKRLEEAKQSATMTLPNARKHPLWVRFDARGGNLQDYYLTAEADKPRSRRFVTFSQLIWPSESGWMEKKDVEVELALSRQFYQQVKLLKNDKGKQKIEFKDKGSGSTFNGHLGGAKLQLERGDLEKEEKNFEDGEIGSVYLNVVIDFEPLQEVKNGRVQAPYGQVLQLIRRPNEFPKVTTYKSEQLVEWIKASPQHSAGVESLASGFRVMSIDLGLRAAAATSIFSVEESSDKNAADFSYWIEGTPLVAVHQRSYMLRLPGEQVEKQVMEKRDERFQLHQRVKFQIRVLAQIMRMANKQYGDRWDELDSLKQAVEQKKSPLDQTDRTFWEGIVCDLTKVLPRNEADWEQAVVQIHRKAEEYVGKAVQAWRKRFAADERKGIAGLSMWNIEELEGLRKLLISWSRRTRNPQEVNRFERGHTSHQRLLTHIQNVKEDRLKQLSHAIVMTALGYVYDERKQEWCAEYPACQVILFENLSQYRSNLDRSTKENSTLMKWAHRSIPKYVHMQAEPYGIQIGDVRAEYSSRFYAKTGTPGIRCKKVRGQDLQGRRFENLQKRLVNEQFLTEEQVKQLRPGDIVPDDSGELFMTLTDGSGSKEVVFLQADINAAHNLQKRFWQRYNELFKVSCRVIVRDEEEYLVPKTKSVQAKLGKGLFVKKSDTAWKDVYVWDSQAKLKGKTTFTEESESPEQLEDFQEIIEEAEEAKGTYRTLFRDPSGVFFPESVWYPQKDFWGEVKRKLYGKLRERFLTKAR

SEQ ID NO: 132 - protein, 1125 aa, unidentified; identical to SEQ ID NO: 47
  source 1..1125: note = Description of Unknown: Cas12 sequence

SEQ ID NO: 133 - protein, 1352 aa, unidentified; identical to SEQ ID NO: 128
  source 1..1352: note = Description of Unknown: Cas12 sequence

SEQ ID NO: 134 - protein, 767 aa, unidentified; identical to SEQ ID NO: 112
  source 1..767: note = Description of Unknown: Cas12 sequence

SEQ ID NO: 135 - protein, 1202 aa, unidentified; identical to SEQ ID NO: 103
  source 1..1202: note = Description of Unknown: Cas12 sequence

SEQ ID NO: 136 - protein, 1375 aa, unidentified; identical to SEQ ID NO: 35
  source 1..1375: note = Description of Unknown: Cas12 sequence

SEQ ID NO: 137 - protein, 1496 aa, unidentified
  source 1..1496: note = Description of Unknown: Cas12 sequence
MADDLSTQRAYTLRLQGTDPEDQSWRDALWMTHEAVNAGGRAFGDWLLTLRGGIAHELADTPVKGKKDITDELRKKRRILLALSWLSVESRRGAPDKFIVAGGEEPAGSRNEKVLQALKEILKRRGLSAEESESWMSDCRASLSAAIRDDAVWVNRSAAFDDAQVRIGASLTREDIWDMLDPFFGSREAYLTPAKKKKEDEDSSEGTGEEKAKDLVQKAGQWLSSRFGTGKGANFDAMAEVYSKISEWAGTAQEGVSGKEGIKNLADALAAFSPVSQNLEGVLKLISGPGYKSATRNLLGELDSLPVVSRDHLSALHEKAAEDTVKCKESTGTKGRRPYADAILNDVEKRCGFTYLTDSDNRSVSILDTSEFPSDYKWGTARHSEFAVILDHAARRISVAHSWIKLAEAERDRCEEDAAKVYDLPDKVKEWLDTFCSNRSDISGAQGEGYRIRRKAIEGWKEVVASWGRSSCITAEDRVAAARALQDDPEIDKFGDIQLFEILAQDEALCVWHKDGDVAKSPDAQMLIDYVLASDAESKKRRFKVPAYRHPDALLHPIFCDFGNSRWDITYDIHGARGKKKAKRGSKKEEAMPRGVAMKLWTGSDVLSVSLRWQSKKLAADLALDQEAEEVTDTAAVSRADRLGRAAAGIDRGAGVTIAGLFEEAHWNGRLQAPRQQLEAIAAVRDNQKLSSEERERRIAFMKDRIRWLVTFSAKLRPQGPWHSYAPTQGLQSDPKYWPHSEINKKRKGQAKLILSRLPGLRILSVDLGHRFAAACAVWETMSSEAIQEACRLANHQLPAPADLYLHLKRTVQKNLIDGEKTVEESTVYRRIGADRLPDGTAHPAPWARLDRQFLIKLQGEEKVREASNEEVWQVHLMESALGLSFPLIDRLVYAGWGGTEKQAARLEALREKGWKPTGTPADQDEEGGGYKPSLAVDELMFSAVRTLRLALKYHGDRARIAFALTADYKPMPGDTRYYFSEAKDRSSGADAAEREAKHKDYLLDMLLLWHDLAFSRKWRDEEAKELWNLHIAALPGYQAPAAPIQEEAGQGRKKAREEARAKMTPAAEALLADGTLREKLHGLWKERWEKDDAQWKKHLRWMKDGILPRGGRAATPSIRYVGGLSLTRLATLTEFRRKVQVGFYTRLFPSGEKREIKEAFGQTALDALERLREQRVKQLASRIAEAALGAGRVSRTALKQDPKRPEARVDAACHAVIIENLEHYRPEETRTRRENRGLMNWASSKVKKYLSEACQLHGLFLREVPAGYTSRQDSRTGAPGMRCQDVTVKTFLNSPFWQKQCVQAQKNKSTARDRFLCALKEAVAQGGMEEEKKMGPIRVPVPGGEVFVSADAASPAAKGLQADLNAAANIGLRALLDPDWPGKWWYVPCDRKTAYPAKEKVEGSAAVDVKQALPFVLPEEKENKGKTKGGKKGKGEVMNLWRDVSAEPLMTGQWLDYTAYRKEVENRVIQVLTAQLKARNPLRFGNLGDEEEIPY

SEQ ID NO: 138 - protein, 1108 aa, unidentified; identical to SEQ ID NO: 131
  source 1..1108: note = Description of Unknown: Cas12 sequence

SEQ ID NO: 139 - protein, 1098 aa, unidentified
  source 1..1098: note = Description of Unknown: Cas12 sequence
MPVRSINLKIVISRNTQGEKSRQSIWTTHAAVNDAVRYYEEQLLIMRGLGYHISDKDVVSKESIQQERLSRIRRAQLENGLPEPLGTDAELNSLVRKFYEFIVPSSVKEDGNAQQANGFLSPLTDPISIGYLSIFEKLGTIPDWVGQLKAGDPQAVENAKKWSATSAGIKRLSETGAPPKWKKLFLTGDPSWPQSFSEDIDKKIKEIEGAPKVICQLMEMGVLPLFPAYFADKLEGSDGSLSRWDRLAFRLAVGHMLSWESWCIKSAEDHFERKRRVESFSEKHTTPSLIICFETLEKYQKERQEKELGQNRSLPMQRPFRITRRQIRGWEDLRDKWLKNTTRTYDSLKSIASKEQTKKGGRFGDPHLFLWLAKPENHAVWDADEDALSIFAKMNAMRGLLERSRETAYMTLPDPIEHPRSIQWEAEGGSNFKNYVITHSPVEGLHVQLPLLCKSESGKLIDQTFEFPLAPSDQFKVAQISKTKSEVTITHQSVLDEEYRSKVGAADLLMDWPYLKNRRFESVEHGDIGPVFLKLSLDIERILPDGWTPKRPQAISHFSSASGNSKHKLSVVSGLRVLSVDLGIRSFGACSVFELSEHKPTSGMSFEIEGLNLWANHERSFMLNLPDEDVGNKGRQLQKTKDAELRAMRRVLGRYRKIYALAGIDPEDRKDILELLCQDQDIFEFERTIYKGLVTSTSVSQPLWEGKIKESLKALRNAFGRKVREWRRANRLNSNLKYAGKTMWAIQHLEDTRRFLHSWSHLGRFSGEIRRADRVKRGVFATRLLQHLDSVKRDRLKTGADLLVQSARGFLRDNQGNWKKSYAPCQVILFEDLSRYLMQTDRPRRENSQLMKWSHRSIPLEVAMQGELYGIHVCDTSAAFSSRYHARLATPGIRCHALRKEDLSNQFLIESLQKENPDIDFGICKAGDLIPRGGGEIFVSCDGNGGISRIHADINAAQNLQRRFWLRHGEAIRIPARKITLKGDEIWVPRSIGKRLQGAMSGCGYLIPTGHESGSCRWERITASKWESISRSSVAQKEEVNEDLLDIALLEEEALELSNEYTTFFRDPSGITLPSDLWFPMKTFWGMTRAKIKSAIKQ

SEQ ID NO: 140 - protein, 1130 aa, unidentified
  source 1..1130: note = Description of Unknown: Cas12 sequence
MTVKSIKVKLRLDNMPEIRAGLWKLHTEVNAGVRYYTEWLSLLRQENLYRRSPNGDGEQECYKTAEECKVELLERLRARQVENGHRDPAGSDDELLQLARQLYELLVPQAIGAKGDAQQIARKFLSPLADKDAVGGLGIAKAGNKPRWVRMRDAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAAYNSILRKLNHAKMFATFTLPDATAHPIWTRFDKLGGNLHQYTFLFNEFGEGRHAIRFQKLLTIEHGVAKEVDDVTVPISMSAQLDDLLPGESNEPTELSFRDHGTDQHFTGEFGGAKIQYRRDQLDHVHRRRGARDVYLNLSVRVQSQSEARGERRPPYAAVFRLVGDTHRAFAHFDKLSNYLAEHPDDGKLGSEGLLSGLRVMSVDLGLRTSASISIFRVARKDELKPNSEGRVPFFFPIKGNDNLVAVHERSQLLKLPGETESKDLRAIREERQRILRQLRTQLAYLRLLVRCGSEDVGRRERSWAKLIEQSVDAANHMTPDWREAFEGELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFATTLREHIDHAKEDRLKKLADRIIMEALGYVYALDAERGKGTWVAKYPPCQLILLEELSEYRFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRLTGKRTADSYGNKVFYTNTGVTYYERERGKKRRKAFAQEELSEEEAELLVEADEAREKSVVLMRDPSGIINRGDWTRQKEFWSMVNQRIEGYLVKQIRSRVCLPESACENTGDI

SEQ ID NO: 141 - protein, 1333 aa, unidentified; identical to SEQ ID NO: 95
  source 1..1333: note = Description of Unknown: Cas12 sequence

SEQ ID NO: 142 - protein, 606 aa, unidentified; identical to SEQ ID NO: 54
  source 1..606: note = Description of Unknown: Cas12 sequence

SEQ ID NO: 143 - protein, 1193 aa, unidentified
  source 1..1193: note = Description of Unknown: Cas12 sequence
MKRIAKFRHDKPVKREAWSKGYRVHKNRIINKVTRSIKYPLVVKDEWKKRLIDDAAHDYRWLVGPINYSDWCRDPNQYSILEFWIDFLCVGGVFQSSHSNICRLAIQLSGGSVFEQEWKDLSPFVRANLIQGIKPAEFIGFLTAEFRSSSNPKNFISKFFEGSNEDLESLTNEFASIVDFIKAKDISLLRKSLPSCKKIAPNLWEKAVGSHSTNELLKLLTKYTRVMLVAEPSHSDRVFSQTVLQSNDQDDPELTGPLPSHKVGKASYLFIPEFIREVNLDKISKLDLSAKSKLAVEQVKKLSELTSDFKQIENQSEAYFGLSTSFNELSNFLGILIRTLRNAPEAILKDQIALCAPLDKDILKITLDWLCDRAQALPENPRFETNWAEYRSYLGGKIKSWFSNYENFFEIPQAASSQQNNNREKKLGNRSAIRALNLKKEAFEKARETFKGDKGTLEKIDLAYRLLGSISPEVLQCDEGLKLYQQFNDELLVLNETINQKFQDAKRDIKAKKEKESFEKLQRNLSSPLPRIPEFFGERAKKGYQKARVSPKLARHLLECLNDWLARFAKVEESAFSEKEFQRILDWLRTSDFLPVFIRKSKDPPSWLRYIARVATGKYYFWVSEYSRKRVQIIDKPIAQNPLKELISWFLLNKDAFSRDNELFKGLSSKMVTLARIMAGILRDRGEGLKELQAMTSKLDNIGLLHPSFSVPVTDSLKDAAFYRAFFSELEGLLNIGRSRLIIERITLQSQQSKNKKTRRPLMPEPFINEDKEVFLAFPKFETKNKVKGTRVVYNSPDEVNWLLSPIRSSKGQLSFMFRCLSEDAKIMTTSGGCSYIVEFKKLLEAQEEVLSIHDCDIIPRAFVSIPFTLERESEETKPDWKPNRFMGVDIGEYAVAYCVIEKGTDSIEILDCGIVRNGAHRVLKEKVDRLKRRQRSMTFGAMDTSIAAARESLVGNYRNRLHAIALKHGAKLVYEYEVSAFESGGNRIKKVYETLKKSDCTGETEADKNARKHIWGETNAVGDQIGAGWTSQTCAKCGRSFGADLKAGNFGVAVPVPEKVEDSKGHYAYHEFPFEDGLKVRGFLKPNKIISDQKELAKAVHAYMRPPLVALGKRKLPKNARYRRGNSSLFRCPFSDCGFTADADIQAAYNIAVKQLYKPKKGYPKERKWQDFVILKPKEPSKLFDKQFYRPN

SEQ ID NO: 144 - protein, 606 aa, unidentified; identical to SEQ ID NO: 54
  source 1..606: note = Description of Unknown: Cas12 sequence

SEQ ID NO: 145 - protein, 1108 aa, unidentified; identical to SEQ ID NO: 131
  source 1..1108: note = Description of Unknown: Cas12 sequence

SEQ ID NO: 146 - protein, 1110 aa, unidentified
  source 1..1110: note = Description of Unknown: Cas12 sequence
MVVRSIKLKMKTNSGTDSIYLRKALWRTHRLINEGIVYYMSLLTLYRQDTLGDRTKEEIQSELIKKIREQQRNNGLSEELGSDQEILSLLRHLYELIIPSINGESGDANQLGNKFLYPLVDPNSQSGKGTSNAGRKSKWKRMKEEGNPDWEVEFKKDEERKANDPTIKVFDHLKKYSLLPLFPLFTNNQKDIEWLPMGKRQSVRKWDKDMFIQAIERLLSWESWNRRLGAEREKLEEKIENFYKEHLSGGQIWIEKIREFERVRDRELGETSFSSNDGYLITSRQIRGWDRVYEKWSKISESASKEELWRVVAEQQSKMREGFGDPKVFSFLAELENMDIWRKHPERIYHIATYNGLLKKLSHTKAQATFTLPDAVKHPLWIRYEAQGGTNLNLFKLEETTKKNCKVILSKIIWPTEDGWFEKENVEVDLAPSKQFYRQIKLQDHIKGKQEISFSDYSSGISLKGVLGGSRIQFDRKYIENHQELLPSGDIGPVFFNLVIDLLPIQETRNGRLKSPIGKTLKVVSSEFPKVIDYKPKELTEWINVSSVTGKVGVESITEGMRVMSIDLGQRTSASVSIFEVVKELPKDKEKMLYYNIKDTELFALHKRSFLLNLPGEEVTKRNRQKRKDRRKKLLFIRSQIRMLASVLKLETKNTPDERKKAINKLVEIVDSYEWTESEKEIWNSEFEYLTNKAVFKQEIWRESLIKSHHRMEPHVGQLVSEWRKSLNEGRRNLAGITMWNIEELEDTRRLLISWSKRSRTPGEANRINNDEPFGAKLLEHIQNVKDDRLKQLANLIVMTALGYKYDKEEKSRDKRWKEKYPACQVILFENLNRYLFSLDRSKRENSKLMKWAHRSIPRTVWMQGEMFGLQVGDVRSEYSSRFHAKSGAPGIRCHSLNDEDLKEESFKLKHLIETGFISEEEISSLKKGDIVPWPGGELFVTLSKPYKKGKDIELTVIHADINAAQNLQKRFWQQNSEVYRIPCQLEKAGDDEFFIPKSQTEIVKKYFGKGRFVKINDKKEVYNWEESEKMKIKTESTITLQDLEGFEDIFQTLELAQEQQKKYSTLFRDPSGYFFNEKNWRPQKEFWSIVNNIIRSSLKKKILKNKVEV

SEQ ID NO: 147 - protein, 687 aa, unidentified
  source 1..687: note = Description of Unknown: Cas12 sequence
  VARIANT 156..527: note = MOD_RES - Any amino acid
MSNPNIPNISPNITLTRDDVVNLLMSSIAMEELGLAHIINAEGEKIQFALGTLQGASGPPATLQQVLEVNQSTQAMLDTIFRQEMMLDSKLKTATNIPTLRGPTGPVGPTGAPGGVISINGQTGVVTLDASNGVMPFMREQSTSSLDDYKDPGIYXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXGATGATGATGATGPQEPRALREPRAPQARREPRGLLEPQVLRGPQEPRALREPRAPRALREPRALRALRALQELRAPRALRGLQEPRVLQGPRERQVRPEPRVLQGPRERQVRQGLQGLLEPRAKRERQVRPEPREPQERQAPLVQQALLEPQALLGQAL

SEQ ID NO: 148 - protein, 1380 aa, unidentified
  source 1..1380: note = Description of Unknown: Cas12 sequence
MNRIYQGRVSKVEIPNPGDKGKPWQPLPNWQDILWQHHELFQDAVNYYLLALLSLARDSANPATPIRKRMDDPASEHQIWTSFRRRGQMRSGMRDSVAKYLCPVKTEPTLDECFAAVLEGNKENPDVLDLALQELLDECDGDGAIQQEGRSMLPRFCSPSYKGDFPQSAAAKDKKSAKEWLPTFLHSAENASNLKLVRQRLKFELFANRDLTGRILDAVESRKRLTEMLGWLTERNPSLAAERERLQKIIAALPDTFNLPAWRGGSVNKEALKQRFYAWLIFEHVESSRITFDILRDSFPTPKEKRKSVSETIQSEKPSLELSDDPIKLARGTRGYVFRAFTSLPCWGADNANLLAWKEFDVAAFKEALKALHQVESKSEERNKERERLLQRHSVMRGSIKWKPSPESEEKEPDVLAGDPRIERLEKLLKTELASEYEMSEGQTVEYGLQPRTIRGFRDLRKEWNKIVKPGEPFTEQKKGNLVTALRSYQIENPNVIGSVRLYEALLEKGNWLVWQEADSATVEKWAKQKFASDPLEALTEERQLLRDIERLKQPIRFTPADAVHSRRQFYLAEKGDLSVKNRFDPQNQTLQVPIAIKNDDHWKQQFVKIHFSAPRAVRDQLIDETMEESKEARWQQPMMEALGLSLKLTKNNAEVSLSECTAVSLMPEELASGDRRILLNFPITLETAPMVKALGKASLWDGQFAAYGDDNFYLRWPKDKWPAEKESNAWYRRLTEFSLLSVDLGQRDAGAFAVIGATTGKPTRPASRFIGEANGQSWHASVRATGVLRLPGEDAHVFRSGQWQEELYGERGRSADATEWTEAKRICETLGLKFDEILGGDPQWFSFPELNDRLLFAVRRAQTRLARLQSWSWMIADETRRENIREAILAADDDELSLKAAAEKSLWPVLAEKLASEVNRLRNLIPQQLVLLANRILPLRGRRWEWIKRDDTSGCHVLRETNPGTDTTSKKVCGQRGLSMKRLEQLDELRRRFQSLNRALMQTPGQTAGLGKSKRGIELPDPCPDLLDKTEQLREQRVNQTAHLILAQALGVRLRTPQKGDALREQNNVHGEYETFRLPVDFIVLEDLSRYLSSQGRGRNENSRLMKWCHRAILLKLKQLCESYGLKILETNAAYSSRFCSRTGVAGFRAVELTPDARKEFRWRKHLNRLEKAASGEIKLDREARAESEHVKRLFDMLDQLNAGRKEAGKPLRTLLAPIAGGQIFIPMQGHATQADINAAINLGLRAIAAPDCHAIHVRIRTERKDKTLRVRTGSNREKVRWEGKNPEIQMNKAADLASLTGDRQPNFFPDPSAIACYDRAKIEGVQLPFASGRGLWGTMNIELQWTRVNQLNNDRAEKKWNLTRSLPEQSSEEDYIPM

SEQ ID NO: 149 - protein, 1144 aa, unidentified
  source 1..1144: note = Description of Unknown: Cas12 sequence
MAVKSIKVKLMLGHLPEIREGLWHLHEAVNLGVRYYTEWLALLRQGNLYRRGKDGAQECYMTAEQCRQELLVRLRDRQKRNGHTGDPGTDEELLGVARRLYELLVPQSVGKKGQAQMLASGFLSPLADPKSEGGKGTSKSGRKPAWMGMKEAGDSRWVEAKARYEANKAKDPTKQVIASLEMYGLRPLFDVFTETYKTIRWMPLGKHQGVRAWDRDMFQQSLERLMSWESWNERVGAEFARLVDRRDRFREKHFTGQEHLVALAQRLEQEMKEASPGFESKSSQAHRITKRALRGADGIIDDWLKLSEGEPVDRFDEILRKRQAQNPRRFGSHDLFLKLAEPVFQPLWREDPSFLSRWASYNEVLNKLEDAKQFATFTLPSPCSNPVWARFENAEGTNIFKYDFLFDHFGKGRHGVRFQRMIVMRDGVPTEVEGIVVPIAPSRQLDALAPNDAASPIDVFVGDPAAPGAFRGQFGGAKIQYRRSALVRKGRREEKAYLCGFRLPSQRRTGTPADDAGEVFLNLSLRVESQSEQAGRRNPPYAAVFHISDQTRRVIVRYGEIERYLAEHPDTGIPGSRGLTSGLRVMSVDLGLRTSAAISVFRVAHRDELTPDAHGRQPFFFPIHGMDHLVALHERSHLIRLPGETESKKVRSIREQRLDRLNRLRSQMASLRLLVRTGVLDEQKRDRNWERLQSSMERGGERMPSDWWDLFQAQVRYLAQHRDASGEAWGRMVQAAVRTLWRQLAKQVRDWRKEVRRNADKVKIRGIARDVPGGHSLAQLDYLERQYRFLRSWSAFSVQAGQVVRAERDSRFAVALREHIDNGKKDRLKKLADRILMEALGYVYVTDGRRAGQWQAVYPPCQLVLLEELSEYRFSNDRPPSENSQLMVWSHRGVLEELIHQAQVHDVLVGTIPAAFSSRFDARTGAPGIRCRRVPSIPLKDAPSIPIWLSHYLKQTERDAAALRPGELIPTGDGEFLVTPAGRGASGVRVVHADINAAHNLQRRLWENFDLSDIRVRCDRREGKDGTVVLIPRLTNQRVKERYSGVIFTSEDGVSFTVGDAKTRRRSSASQGEGDDLSDEEQELLAEADDARERSVVLFRDPSGFVNGGRWTAQRAFWGMVHNRIETLLAERFSVSGAAEKVRG

SEQ ID NO: 150 - protein, 647 aa, unidentified
  source 1..647: note = Description of Unknown: Cas12 sequence
  VARIANT 35..498: note = MOD_RES - Any amino acid
MGLVDTAGGFLIPAALDPAILLSGDGSTNPIRQVXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXPSRCRCLAFGRSSSAIRRPAASSSEGVKSLRSVFSERSALARSTTSAIVWHTCTGMRSHPSSRAASSRRWPNTSRLSGVTPIGCSKRQGDSPCGCCCGAHLVVAQRCVAFCCHDRGVTEKLLYSSQVVGAAVGAGGVSVPQGVHGRVTG

SEQ ID NO: 151 - protein, 1262 aa, unidentified; identical to SEQ ID NO: 45
  source 1..1262: note = Description of Unknown: Cas12 sequence

SEQ ID NO: 152 - protein, 978 aa, unidentified
  source 1..978: note = Description of Unknown: Cas12 sequence
MQEIKRINKIRRRLVKDSNTKKAGKTGPMKTLLVRVMTPDLRERLENLRKKPENIPQPISNTSRANLNKLLTDYTEMKKAILHVYWEEFQKDPVGLMSRVAQPAPKNIDQRKLIPVKDGNERLTSSGFACSQCCQPLYVYKLEQVNDKGKPHTNYFGRCNVSEHERLILLSPHKPEANDELVTYSLGKFGQRALDFYSIHVTRESNHPVKPLEQIGGNSCASGPVGKALSDACMGAVASFLTKYQDIILEHQKVIKKNEKRLANLKDIASANGLAFPKITLPPQPHTKEGIEAYNNVVAQIVIWVNLNLWQKLKIGRDEAKPLQRLKGFPSFPLVERQANEVDWWDMVCNVKKLINEKKEDGKVFWQNLAGYKRQEALLPYLSSEEDRKKGKKFARYQFGDLLLHLEKKHGEDWGKVYDEAWERIDKKVEGLSKHIKLEEERRSEDAQSKAALTDWLRAKASFVIEGLKEADKDEFCRCELKLQKWYGDLRGKPFAIEAENSILDISGFSKQYNCAFIWQKDGVKKLNLYLIINYFKGGKLRFKKIKPEAFEANRFYTVINKKSGEIVPMEVNFNFDDPNLIILPLAFGKRQGREFIWNDLLSLETGSLKLANGRVIEKTLYNRRTRQDEPALFVALTFERREVLDSSNIKPMNLIGIDRGENIPAVIALTDPEGCPLSRFKDSLGNPTHILRIGESYKEKQRTIQAAKEVEQRRAGGYSRKYASKAKNLADDMVRNTARDLLYYAVTQDAMLIFENLSRGFGRQGKRTFMAERQYTRMEDWLTAKLAYEGLPSKTYLSKTLAQYTSKTCSNCGFTITSADYDRVLEKLKKTATGWMTTINGKELKVEGQITYYNRYKRQNVVKDLSVELDRLSEESVNNDISSWTKGRSGEALSLLKKRFSHRPVQEKFVCLNCGFETHADEQAALNIARSWLFLRSQEYKKYQTNKTTGNTDKRAFVETWQSFYRKKLKEVWKPAV

SEQ ID NO: 153 - protein, 1488 aa, unidentified
  source 1..1488: note = Description of Unknown: Cas12 sequence
MGKQSTNSSGLKATSGAPSIEKPVTTQRAYTLRLRGIEGDKTWRDSLWATHEAINLGAKAFGDWLLTLRGGLEHTLADATVAGGKGKPARPPSADERRDRRVLLALSWLTVEDERGAPKAPGLIVAYGDDCKSAKDSQDGRDRKVEDALRDILIKRSVAPGAVESWVNDCVVSLKARIRDDAVWVNRSAAFDALRASWRGLTCPNARTVLEQFFGPVADWITLPARAADDGEGDTGGPTAAGSSNDTEFKLVARSFLSENFGTGLKSSKSDISDALMQATRSLAALRGGSPGSAVWSHLCTEFGLLAADDEARAKALRVRIGWTSGRRSKGRLALATAASKASLAQVDIELLIKKLSEEAQEKLAGSTKLPLPWVNDLMAALERSIGFGFVTERNLIDEFGVMLDHAARRVSIACSWIKLAELERRQFEEDAKKLDQVRTQHADAAAFLDTLGCRRGSESGSAGGASVVIRKRAVLGWNEVVGDWARQGCKSVQERIDAARLLQGELEKFGDIRLFEELAANDAQVVWQNAEGETDPTILARYSAGSVAKANQQRFKVPAYRHPDPLRHPVFGDFGKSRWKIDFAVHESDRAQSGGKRVRSLDAAWQSDVRNMRMALWTGKRVEMVPLRWSSKRLSKDLGISDLSLKDPTVVTRGDRLGRAAVGPGDPVRVASVFAEDNWNGRLQAPRVELNRVARLTQSGNLVQARRLRDRLSWLVSFSPKLAPSGPFIDYAAAHGIEPHKKSGEYWPNSAMNKERKGHHSKLIYSRLPSLRVLSVDLGHRFAAACAVWESMSAAQMRLAASKGKVVRGGIAEGALFIHIESTVADGTVRTTVYRRIGEDALPDGSPHPAPWAKLERQFLIKLQGEETPSRMAALDELVMIADWERALGYQPVTASSTKQSNGVAGLMGRTVRLFMHASRRHFDRARIAHNLTAEHRTKPGGVPESLTEETRIELLIDTLALWHGLFAGDRWRDPRAQKEWETSGLPSLKLPGRGDDDETAFGGPGRKAAMVVYRNELKPHAERMAHSDLSNLSKCWTDRWAQDDREWTAKSGRLRSLRRWITPRGLRPVSGDSAQLIERKAAAALRARHVGGLSMQRINTLTDLYRLLKSFKNRPEPSNLRKNIPEKGDARLIGFNQRLLDVRDRLREQRVKQLASRIVEAALGIGRIKIRRVAAGSPRPTLRVDAACHAVVTENLSNYQPAELQTRRENRQLMAWASSKVHKYLAESCQLHGLHLREVQPNYTSRQCSRTGAAGLRGVAVSATDLLTNPWWMRDVAQAKARIEKALKNGRNGAVADQLLVSAEAWALRIPERDRHDRKADESVKAFHAERVILPRKGGDLFVAESARCNGKGHVPALQADLNAAANVGIRALLDPDWPGKWWWVPCVGGTYSPSPEKTSGAKVLEHIDQLPHEMVDPPQVAPKSSAPQPKSSRAAKVKAKEFRAIENRWRTCSSELLSQGKWIPSGEYWSKVEAAVCLVLNDQIT

SEQ ID NO: 154 - protein, 1400 aa, unidentified
  source 1..1400: note = Description of Unknown: Cas12 sequence
MAAFQRSYTMNLKPATSEQDKFILWNRLFLTHWSVNEGAKIFGELFLNLRGGLSPELDIFDLDKVKDDKKKKAFVMGRRRLLALGWLSVEDNLSAGEHPFRIREIPVGRNMGKSQASTLLTEILKNKGIKDEAVIKEWIDDCTPSLIANIREDAVWINRASSFNSITPCPTKDEVWIVLSGLLGLRFLDLSLEEVKGKETEYLYDLGEKETQSKSDPSKKARELFGNLFTQNPVLMKNSRDKKDTFAKEFYLAFKEFKDYEKLKEKIESWRKEKEFPLIENPVAEKYPPEVTFTGSPCTVSKRYRKLLVSLELWPSSQDENGNIPKTEKTEDKTHNQVLLDYLLKACNEGNKGTQKIITPVWANNLKAELELKMNEIIRIGESSSTELQRLMIKMAARRISQTLSWIKINEQTKHDAYQKKNKAFKLLSEIDKNGEACKWLENYELFRTDDSGGEEYHISLRAISCWKQILEEWQKNDSPKALREKVKEVQAEEEKFGDARLFEDLADDNARSVWLLPDGNKTPDILNWWCEYRTAEIDESRFKIPCYCHPHPFKHPVYVEYGKSNPKVIFAMKNNKVKKGHIEHGWNPQNPRSIALSLFNNGNRESSLVPFIWESKRLWKDLGGEATQIGDIPRSDRMGLSGKRESVKPKAPFQKEVWNARLQSDRRTLEKLEKYWNPESMKWIDDGKFLIQSKWFITFGPDMETAEGPWKLYLKEKYVDNKILGNRSKENQKRGYRAKKLLSGYPAGMRILSVDLGHRYAASCAVWETITKKQITEELAYQPDNNSLFEHSCKTIDKKIKNTVYRRIGEDSIDAPWAKLEKQFTIKLQGEDKSCYLLRSDEKELFRSILSKLSCLNNDTGHNILEMIENLLRIVKAKIYRQGILARISYSMTAQYKPGKGGQKSPLSDEDKIHYLSENLAAWSAIMGNQEWNEDVISDWYKTYISHLVSGPKPKEGNRKSDRDKIIEYFLPAARKLYDDNETRIKIHDLFKELWDENNKQLSAVLKEIKKIILPKGIRYFDKNTDNPSKWKNNQSKLKQITHRGGLSMQRIVAIEEYYKLAKAYKNHPEPDDLTKNIPLPGDNSSAGFNQRIRDTLERMKEQRVKQIASRIVESALGLGIEGYKKRPLTPENKPCQAIVIEDLSHYRPDELQTRRENRRLMQWSSSKVKKYLKEACEMHDVRLVEISPEYTSRQDSRTGAAGLRCIDINIREFLKDSSRWQNKINTIQKKPANKKSNLDQYLIELNESLGNKYKDKVIPSDNFVRIPRKGGDVFVSSSKESPVSKGIQADLNAAANIGLKALLDPDWAGAWWYILIEVKSNHVIPYGEKYKGSECLRAWKFSGLENQVMKNNMNLWRDLQSQFSGEDKWMSYKEYNELTEKRVINILRERAGLELIKE

SEQ ID NO: 155 - protein, 1263 aa, unidentified; identical to SEQ ID NO: 72
  source 1..1263: note = Description of Unknown: Cas12 sequence

SEQ ID NO: 156 - protein, 1193 aa, unidentified; identical to SEQ ID NO: 143
  source 1..1193: note = Description of Unknown: Cas12 sequence

SEQ ID NO: 157 - protein, 1108 aa, unidentified; identical to SEQ ID NO: 131
  source 1..1108: note = Description of Unknown: Cas12 sequence

SEQ ID NO: 158 - protein, 1283 aa, unidentified; identical to SEQ ID NO: 46
  source 1..1283: note = Description of Unknown: Cas12 sequence

SEQ ID NO: 159 - protein, 1413 aa, unidentified
  source 1..1413: note = Description of Unknown: Cas12 sequence
MNRIYQGRVTKVEKLKNGKSPDDREELKDWQTALWRHHELFQDAVSYYTLALAAMAEGLPDKHPINVLRKRMEEAWEEFPRKTVTPAKNLRDSVRPWLGLSESASFGDALKKILPPAPENKEVRALAVALLAEKARTLKPQKTSASYWGRFCDDLKKKPNWDYSEEELARKTGSGDWVAGLWSEDALNKIDELAKSLKLSSLVKCVPDGQINPEGARNLVKEALDHLEGVSNGTKKEKNDPGPAKKTNNWLRQHASDVRNFIHKNKNQFSSLPNGRLITERARGGGININKTYAGVLFKAFPCPFTFDYVRAAVPEPKVKKVDQEKKSEQSATWTELEKRILRIGDDPIELARKNNKPIFKAFTALEKWSDQNSKSCWSDFDKCAFEEALKTLNQFNQKTEEREKRRSEAEAELKYMMDENPEWKPKKETEGDDVREVPILKGDPRYEKLVKLFGDLDEEGSEHATGKIYGPSRASLRGFGKLRNEWVDLFTKANDNPREQDLQKAVTGFQREHKLDMGYTAFFLKLCERDYWDIWRDDTEVEVKKIREKRWVKSVVYAAADTRELAEELERLQEPVRYTPAEPQFSRRLFMFSDIKGKQGAKHIREGLVEVSLAVKDQSGKYGTCRVRLHYSAPRLIRDHLSDGSSSMWLQPMMAALGLSSDARGCFTRDSKGNVKEPAVALMSDFVGRKRELRMLLNFPVDLDISKLEENIGKKARWEKQMNTAYEKNKLKQRFHLIWPGMELKETQEPGQFWWDNPTIQKEGMYCLAIDLSQRRAADYALLHAGVNRDSKTFVELGQAGGQSWFTKLCAAGSLRLPGEDTEVIREGKRQIELSGKKGRNATQSEYDQAIALAKQLLHNENSAELESAARDWLGDNAKRFSFPEQNDKLIDLYYGALSRYKTWLRWSWRLTEQHKELWDKTLDEIRKVPYFASWGELAGNGTNEATVQQLQKLIADAAVDLRNFLEKALLHIAYRALPLRENTWRWIENGKDGKGKPLHLLVSDGQSPAEIPWLRGQRGLSIARIEQLENFRRAVLSLNRLLRHEIGTKPEFGSSTCGESLPDPCPDLTDKIVRLKEERVNQTAHLIIAQSLGVRLKGHSLFTEEREKADMHGEHEVIPGRSPVDFVVLEDLSRYTTDKSRSRSENSRLMKWCHRKINEKVKLLAEPFGIPVIEVFASYSSKFDARTGAPGFRAVEVTSEDRPFWRKTIEKQSVAREVFDCLDNLVGKGLNGIHLVLPQNGGPLFIAAVKEDQPLPAIRQADINAAVNIGLRAIAGPSCYHAHPKVRLIKGESGTDKGKWLPRKGKEANKRENAQFGNVDLDLEVKFNRLDIDSDVLKGDNTNLFHDPLNIACYGFATIQNLQHPFLAHASAVFSRQKGAVARLQWEVCRAINSRRLEAWQKKAEKAAVKR

SEQ ID NO: 160 - protein, 1408 aa, unidentified
  source 1..1408: note = Description of Unknown: Cas12 sequence
MSHELAEQPSPPKNKEPTCEESDAIRKNRRILLALSWLSVEDDCSAPTGTFRVASGKDSEAERKNKVLTAFRSILTARRMRSQDVESWIADCAASLSAKIREDAVWINRSACFDQRALDLKVLSREYAKAAVMSFFGPLDEYFKLPDEADDTKPAVGGDGPDFRTLARQWVSTNFGTGKKSDSEAIAQNLRKLADANLAPFSGKPKAALIAHLSVELDGSTADIDGLCRAIGWNTGRPSKGRVAIERLPDPPTETSIQTMQQKFREEAEAKASSKGLRQVPEWMPAFQKSIERDCGMPFKLGEGRDHIGEFSVMLDHAARRVSIGHSWIKRAEAERRRFEADAQRLNHIPAAAKDWLDQFVQFRSGSSGAAAAGGEYRIRRRAIEGWDEIIKRWKRAACKSPEDRVAAAREVQADPEIEKFGDIQLFEALAADDAECVWRGDGNGTPDPLKDYVAATDALDKMRRFKVPAYRHPDPLAHPVFGDFGNSRGDIRFAVHEAAKATRGTKRIAKDQKEWIRERHGLRMGLWDGQSVRTADLRWSSKRLVDDLALRNHVTTRRTGPVSRADRLGRAAAGLGADEAACVAGLFELPDWNGRLQAPRAQLDAIAACVAANGGKWDDKARKLRDRIEWLVSFSAKLECCGPFMEYASQNGIQPNGKGEYYPHAERNKGRTGHAKLILSRLPGLRVLAVDLGHRFAAACAVWEALSKIAFDAETKGREVVSGGRAADDLYCHTRHLDCAGKARTTIYRRIGPDKLPDGSDHPAPWARLDRQFLIKLQGEERPARAAGPAETAAVQQIETDLGRARGQEDLPPRPVDSLMREAVRTIRIALRRHGDAARIAYAFKPGAKRLKPGGGAQDHTPETHADAILEALLRWHELATGARWRDPWAETQWKDWVQPHISATLPELANDADRWERKRHRAALEQVLRPVAQMLIQRPTDALHQVWSKHWADEDLKWPSRLRWLRNWLLPRGPRARSGAARNVGGLSLLRIATLRELYQTQKAYAMRPEPDDPRKRIAGRNDDRYDELGRSVLQVIERLREQRVKQLASRIVEAALGVGRAKPTRGRQRPQSRVDVPCHAVIIESLRNYRPDELQTRRENRAIMNWSAGKVRKYLEEACQLHGLHLREVMPNYTSREDSRTGLPGVRCVDVPVDPKLGKPKAYWWNSVLSTARKKSIGDAASHDKQGDATSRFIVELAGCLDRLKADGKPLPKTVRVPRIGGDLFVAAPPTSCTAPAHQPHPACDGARALQADLNAAANIGLRALLDPDFPAKWWYVPCIDDQRGLALPRADKVLGSACFPGDPATFGSLLKTRTAAGPAVDGQAAPDRKPRTGTHRPGSAKSRSLGDGKATTNYWSDRSARDLRPADEGGHWQPTNVYWNWVRKRALLGLYSFNGLSPPSDDRP

SEQ ID NO: 161 - protein, 1108 aa, unidentified
  source 1..1108: note = Description of Unknown: Cas12 sequence
MAIRSIKLKLKTNTGPEAQNLRKGIWRTHRLLNEGVAYYMNMLLLFRQESTDKKTKQEIHEELIRHIRAQQQRNHADEKTQALPLEKALEALRKLYELLVPSSVGQSGDSQIISRKFLSPLVDPNSEGGKGTSKAGAKPAWQKKKEANDPTWKQDYEKWKKRREEDPTASVITTLEEYGIRPLFPLYTNTVAEIAWLPLKSGQFVRTWDRDMFQQAIEGMLSWESWNRRVQEEYAKLEGKMAQLNEQLEGGEEWIRLLEQYEEKREQELRENMTAANDKFRITKRQMKGWKELYEVWSTFLPSASQEQYKEAIKRVQQRLRGKFGDFHFFQYLSEEENRLIWKGNPQRIHYFVARNELTKKLEKAKQSARRTLPDANKHPLWVRYDARGGNLQDYYLTAESDKPRSRRFVTFSQLIWPSESGWLEKKDVQAELALSRQFYQQVTFLKNDKGKQEIEFKDKGSGTTFSGHLGGAKLQLERSVLENKERKFEEGEIGKAYLNVAIDFKPLQEVKNGRVQAPYGQVLQLIRLPNAFPKVRTYKSEELVEWIKASPQHLSGVESLASGFRVMSIDLGLRAAAATSIFSVEESSDKNATKLAYWIEGTPLVAVHQRSYMLRLPGEQVEQHVWEKRDERGDQHKRVRFQIRRLAEIIRLANKQYGDRWDELNRLDEAVSKEKSPLDQADRTFWEGIVSDLTTALPLNDADWTEAVVQIHRKAELYVGKVVQAWRKRFNADERKGIAGLSMWSIEELDGLRKLLISWSRRTRNPQEVNRFEPEHTGHKRLLTHIQNVKKDRLKQVSHAIVMTALGYIYDEKKQKWCAKYPACQVILFENLSQYRSNLDRSAKENSTLMKWAHRSIPKYVHMQAEPYGIQIGDVRAEYSSRYYAKTGTPGIRCKKLREHDVKGWRLDHLKKRLVNEQFLTEAQVEQLKAGDIIPDDSGELFMTMTDGSGGKEIVFLQADINAAQNLQKRFWQRNSELFKVSCRVIVRDEAEYLVPQAKKVQEKLGKGVFVKKSDTAWKEVYVWDSQAKLKGKTTFTEESESPEQLEDLQEMIEEAEEAKGTYRTLFRDPSGVFFPDFVWNTPKDFWGEVKRKLYGKLRERLLTKVR

SEQ ID NO: 162 - protein, 1364 aa, unidentified
  source 1..1364: note = Description of Unknown: Cas12 sequence
MNRIYQGRVTKVEVPDGKDEKGNIKWKKLENWSDILWQHHMLFQDAVNYYTLALAAISGSAVGSDEKSIILREWAVQVQNIWEKAKKKATVFEGPQKRLTSILGLEQNASFDIAAKHILRTSEAKPEQRASALIRLLEEIDKKNHNVVCGERLPFFCPRNIQSKRSPTSKAVSSVQEQKRQEEVRRFHNMQPEEVVKNAVTLDISLFKSSPKIVFLEDPKKARAELLKQFDNACKKHKELVGIKKAFTESIDKHGSSLKVPAPGSKPSGLYPSAIVFKYFPVDITKTVFLKATEKLAMGKDREVTNDPIADARVNDKPHFDYFTNIALIREKEKNRAAWFEFDLAAFIEAIMSPHRFYQDTQKRKEAARKLEEKIKAIEGKGGQFKESDSEDDDVDSLPGFEGDTRIDLLRKLVTDTLGWLGESETPDNNEGKKTEYSISERTLRIFPDIQKQWSELAEKGETTEGKLLEVLKHEQTEHQSDFGSATLYQHLAKPEFHPIWLKSGTEEWHAENPLKAWLNYKELQYELTDKKRPIHFTPAHPVYSPRYFDFPKKSETEEKEVSKNTHSLTTSLASEHIKNSLQFTAGLIRKTNVGKKAIKARFSYSAPRLRRDCLRSENNENLYKAPWLQPMMRALGIDEEKADRQNFANTRITLMAKGLDDIQLGFPVEANSQELQKEVSNGISWKGQFNWGGIASLSALRWPHEKKPKNPPEQPWWGIDSFSCLAVDLGQRYAGAFARLDVSTIEKKGKSRFIGEACDKKWYAKVSRMGLLRLPGEDVKVWRDASKIDKENGFAFRKELFGEKGRSATPLEAEETAELIKLFGANEKDVMPDNWSKELSFPEQNDKLLIVARRAQAAVSRLHRWAWFFDEAKRSDDAIREILESDDTDLKQKVNKNEIEKVKETIISLLKVKQELLPTLLTRLANRVLPLRGRSWEWKKHHQKNDGFILDQTGKAMPNVLIRGQRGLSMDRIEQITELRKRFQALNQSLRRQIGKKAPAKRDDSIPDCCPDLLEKLDHMKEQRVNQTAHMILAEALGLKLAEPPKDKKELNETCDMHGAYAKVDNPVSFIVIEDLSRYRSSQGRSPRENSRLMKWCHRAVRDKLKEMCEVFFPLCERRKAGSAWVSLPPLLETPAAYSSRFCSRSGVAGFRAVEVIPGFELKYPWSWLKDKKDKAGNLAKEALNIRTVSEQLKAFNQDKPEKPRTLLVPIAGGPIFVPISEVGLSSFGLKPQVVQADINAAINLGLRAISDPRIWEIHPRLRTEKRDGRLFAREKRKYGEEKVEVQPSKNEKAKKVKDDRKPNYFADFSGKVDWGFGNIKNESGLTLVSGKALWWTINQLQWERCFDINKRHIEDWSNKQKQ

SEQ ID NO: 163 - protein, 1313 aa, unidentified
  source 1..1313: note = Description of Unknown: Cas12 sequence
MGEENNFSQFTGLYELSKTLRFELKPVWETEKLLIENQVFPKDKIVYESYKKIRPYLDKLHLQFIEESLSSVKLDFNEIEKKFLEWDKEKDKTTKNKLKEEIFWKNKKWWLNSNLRKDMVSYFNLNWKKWINNFENKTIVNEKWKEEKIKIKWEWYKILLSDSNLNILSDFFTQEKEWDNVLIEKYIENPNFKENDWEKKFILEKQNLFKSFKWFTTYFTNFNHSRENFYKDDWKSWRIATRIIDENLIFFLKNKKAFDEKYKNNSEITVKFNEKLLNFWEKLEDFFSLDFYNRCYTWKQIEYYNQLIWELNSVVNHEKQAKFSEYTQNKKVSENNKFNKYDFPIFKELYKQILSEKATEQKFIEINDFKELKQNLQELIEKNKQKNKFAINLTKSLIEKIDEYDFEKIYISKLSLNTISSKFFWSNKWFFIQENLEKNIWKKNKNWKIDLPDFIKLSDIKTALENFNKILFEDKENKENIFKEEFDNITENNIFIKFMKIYENEFINLIEWKKNNKWDYEIIWYNKSLLDLEKNILNISDFSDKKEEKEKQIEYIKKYLDSSLNIYRVMKYFALEKGKESVEWEFETDDIFYNEFKKFYIDNEIISYYNEFRNYLTKKPYSSNKLKLNFENWTLIDGWDKNKEPDNYWTILRKNWKYFLALQIKWKNNIFYKKKWSWVIDIEEAYKIDLWDEFYEKMDYKFLPDPKKMLPKVIFAKSNVKLFNPSKEILQIKENETFKTWDKFKVNDFYKIVDFYKENIIKYPDWKIFNFKFSDTKTYNNLSDFYKEIELWSYDLNFRKVSKKYILKSIEEKNIYLFEIYNKDFADWKTWSENLHTMYFKWLFEENNLNNIVLKLNWQAEIFRREASLKEKEVNRAKENKEKSHNIIENARYTKDKLFFHCPIKLNFAKHNEKINQEILKYISDNKEINIIWIDRWEKHLAYYSVINRDWNIIKDKNWNLVKWSLNIVWNNQNYHDKLETREKERQDARWSWKTIWNIKDLKQGYISQVVHKLAELVIEHNAIIVFEDLNSWFKRWRQKIERQVYQKLEKALIEKLNYLTFKDKNFWENWHYLKAYQLTAPFETFEKVWKQTWVIFYTDPSYTSSTCPACWFRKDLYLKYSNLKNALWDIEKIDSIIFDWKRFIFEYNNKKVFTDRDRKRNISSEESWIKWWKTIDNNITEFLEILFKKSNIDYTSWNNLILDIQEINEKELYKWVFDNFNAILNIRNSTIKDNSRTWDFICCPACDFDSRKENKIWIENWDDNWAFNIARKWIIILNKIDSYLNEKWSLDKILWWDMIVKQIDWDDFTHKK

SEQ ID NO: 164 - protein, 767 aa, unidentified; identical to SEQ ID NO: 112
  source 1..767: note = Description of Unknown: Cas12 sequence

SEQ ID NO: 165 - protein, 1313 aa, unidentified; identical to SEQ ID NO: 163
  source 1..1313: note = Description of Unknown: Cas12 sequence

SEQ ID NO: 166 - protein, 1500 aa, unidentified
  source 1..1500: note = Description of Unknown: Cas12 sequence
MATAINYPTTQRAYTLRLRGIDPQDQSWRDALWATHEAVNRGAKVFGEWLLTLRGGLDHQLADAPVKVRGGTTRLPSDEERRDRRVLLALSWLSVEDAHGAPPDASLIVAKGTDSADCRARKLADALIAILQARSVAASEIGDPSKPPEDQPGTWLGDCMGSLSAAIRDDAVWVNRSKAFDAATQSCPSLTRDEIWDFLEPFFASPDAYLKPERAESDEGDSTSAATEDKAKDLVQKAGGWLSKRMGAGGGANFQDLARAYQAIAQWASSAQPGQSAQQAVGSLAGYLSQHGFSPTANDATGVLAVISGPGYKSATRNHITAIATSPEITPQDLSKLQELATKDKAGCSSKIGGKGPRPYATMILQQVEAACGFTYLQSDGPARHREFSVMLDHAARRVNVAHSWIKNAEAERRQFESDARRIKKVPQDALNWLRGYCEERGGASGSLEGYRIRRRAIDGWDQVVIRWSRSDCQSADDRIAAARQLQDDPEIDKFGDIQLFEALAAEEALCVWKPDGNPTAQPLKDFVAATEADAKKKRFKVPAYRHPDPLRHPVFTDFGNSRWGIEYSAHRAPAKCDELGQQVDRLTQAVAEAQRNLDGATAAQRASRESKLAEAQSKLVAAQTEFAAINDPHRVELKLWNGQAVAAIPMRWSSKRLIADLSLRRATQPSSDQRIGVTRADRLGRAAGNADDGRPVTITGLFQQDHWNGRLQAPRAQLDAIAKHVDKHGWDAKARRQIARIRWVVSFSAELSQQGPWFEFCHRFGEDAPARPFVSRHGEYAVKHRDNDQRKGHAKLILSRLPGLRVLAVDLGHRYAAACAVWEAISSDQMRQACAAANAPAPHPLAMYIHLKSTTAKGKPTTTIYRRIGPDKLPDGTPHPAPWARLDRQFLIKLPGEDRPARAASPDEIKAVEDFEDSVGRVRTAVDPPRKRGVDLLMHDAVRTARLALARHGRRARIAFQLISQVRILPGGRPQTLDDAGRRDLLNDTLADWYALATDSRWTDAAARQLWNERLAALNGGFTIDPPADASQPEAERTRAQRRQAEQELRHRLASLVEALFCNPTLCQQLHQAWTDRWNADDQQWRSRLKWLSRWLLPRGGSRRDGSRRHVGGLSLTRISTLIDFRRKVQVGYFTRLRPDGSRAEIGPQFGQSTLDAIQRLKDQRIKQLTSRIVEAALGIGVEQDRIWDAAKRKWRTVKRPREPRYHVDDQGVQQRDPRFQACHAVVIEDLSHYRPEETRTRRENRATMDWKSAETRKRLADHCQLYGLHLRDVNPQYTSRQDSRTGAPGCRCVDVSVADFLTKPAWRKQVAQARGKVASNRGDARDRLLVELDHQLTTANSLRGEMDSLRIPVNGGEVFVSADPRSPLAAGIQADLNAAANIGLRALMDPDFLGTWWYVPCDPSTKKPHIEKVKGSILATVGALQATSEEAAAPPRRGRGGTRSAAPREVINLWRDPSAVRIQDATAGEVWDVTPVYWSIVKDRVVDVLRQRNTKSGD

SEQ ID NO: 167 - protein, 1202 aa, unidentified; identical to SEQ ID NO: 103
  source 1..1202: note = Description of Unknown: Cas12 sequence

SEQ ID NO: 168 - protein, 1397 aa, unidentified
  source 1..1397: note = Description of Unknown: Cas12 sequence
MAAFQRSYTMNLKPATSEQDKFILWNRLFLTHWSVNEGAKIFGELFLNLRGGLSPELGIFDLNKDKDDRKKKALVMGRRRLLALGWLSVEDNLSAGDHPFRIREIPVGRNMEIKQATTLLTEILKNKGVKDEAVIKEWIDDCTPSLIANIREDAVWINRAKSFYSMNPCPTKDEVWKILSYVLNTSFLDLSLNDSSERDNTKNKKGTKENEKDVSNKSKELYGWLFTKNPNKMREAGENKDKFINNFRENFNTFTDYANLKVEIELWRKNNISNTLLITQKAKYPPEVKEANHPSKFSVGYRKLLVHLELWPSSKDENGDIPKGIEGKDKSHNQILLDYLLEVCNEGNKTTKKVIVPAWADGIKTELESKASIKVGDSTSSVLQRLMIKMAARRISQTLSWIKINEQVRHDAYQKKNKAFKLLCEIDKNGEACKWLENYELFRRDDSGGEEYHISARAISCWKQILEEWQKNDSSKALREKVKVVQAAEDKFGDARLFEDLADDNARSVWLLPDGNKTPDILNWWCEYRTADIDESRFKIPCYCHPHPFKHPVYVEYGKSNPQVIFSLKHDKARKNRIDNGWNPKNPRILALLLLDIVRQKSTLAPFVWESKRLWKDLGGDATVTYKIPRSDRMGLSSIGNIDYARPEVPFLKEKWNARLQSDRRTLEKLEKYWNPESMKWIDDGKFLIQSKWFITFGPDMETAEGPWKLYLKENINDNNYLGNRSKENQKRGYRAKKLLSGYPAGMRILSVDLGHRYAASCAIWETITKKQITEELAYQPDKNSVFEHSCKTIDKKIKNTVYRRIGDDSIDAPWAKLEKQFTIKLQGEDKSCYLLRSDEKELFRSILSKLSCLNNDTGHNILEMIENLLRIVKAKIYRQGILARISYSMTAQYKPGKGGQKSPLSDEDKIHYLSENLAAWSALMGNQEWNEDGISDWYKKYISHLVSGPKPKEGNRKSDRDKIIEYFLPAARKLYDDNETRINIHDLFKELWDENNKQLSAVLKEIKKIILPKGIRYFDKNNDSSSRWKNNQSKLKQITHRGGLSLRRIVAIEGYYKLAKAYKNHPEPDNLTKNIPLPGDNSSAGFNQRIRDTLERMKEQRVKQIASRIVESALGLGIEGYKKRPLTPESKPCQAIVIEDLSHYRPDELQTRRENRRLMQWSSSKVKKYLSEACEMHDVLLVEISPEYTSRQDSRTGVAGLRCIDINIREFLKDSSSWQNKIKTIQMKPTNKKSNLDQYLIELNESLGERYKDKVIPSDKFVRIPRKGGDIFVSSSKESPVSKGIQADLNAAANIGLKALLDPDWAGAWWYILIEAKSNHVIPYGKKYKGAECLRDFKFSGLENQVMKNNMNLWRDLQSQFSSEDKWMSYKEYNELTEKRVINILRERAGLELIEE

SEQ ID NO: 169 - protein, 1203 aa, unidentified
  source 1..1203: note = Description of Unknown: Cas12 sequence
MPTRTINLKMVLGRKDDTAELRRALWTTHEHVNLAVAEVERVLLRCRGRSYWTLDRRGDPVHVPESQVAEDALAMAREAQRRNGWPVVGEDEEILLALRYLYEQIVPSCLLDDLGKPLKGDAQKIGTNYAGPLFDSDTCRRDEGKDVACCGPFHEVAGKYLGALPEWATPISKQEFDGKDASHLRFKATGGDDAFFRVSIEKANAWYEDPANQDALKNKAYNKDDWKKEKDKGISSWAVKYIQKQLQLGQDPRTEVRRKLWLELGLLPLFIPVFDKTMVGNLWNRLAVRLALAHLLSWESWNHRAVQDQALARAKRDELAALFLGMEDGFAGLREYELRRNESIKQHAFEPVDRPYVVSGRALRSWTRVREEWLRHGDTQESRKNICNRLQDRLRGKFGDPDVFHWLAEDGQEALWKERDCVTSFSLLNDADGLLEKRKGYALMTFADARLHPRWAMYEAPGGSNLRTYQIRKTENGLWADVVLLSPRNESAAVEEKTFNVRLAPSGQLSNVSFDQIQKGSKMVGRCRYQSANQQFEGLLGGAEILFDRKRIANEQHGATDLASKPGHVWFKLTLDVRPQAPQGWLDGKGRPALPPEAKHFKTALSNKSKFADQVRPGLRVLSVDLGVRSFAACSVFELVRGGPDQGTYFPAADGRTVDDPEKLWAKHERSFKITLPGENPSRKEEIARRAAMEELRSLNGDIRRLKAILRLSVLQEDDPRTEHLRLFMEAIVDDPAKSALNAELFKGFGDDRFRSTPDLWKQHCHFFHDKAEKVVAERFSRWRTETRPKSSSWQDWRERRGYAGGKSYWAVTYLEAVRGLILRWNMRGRTYGEVNRQDKKQFGTVASALLHHINQLKEDRIKTGADMIIQAARGFVPRKNGAGWVQVHEPCRLILFEDLARYRFRTDRSRRENSRLMRWSHREIVNEVGMQGELYGLHVDTTEAGFSSRYLASSGAPGVRCRHLVEEDFHDGLPGMHLVGELDWLLPKDKDRTANEARRLLGGMVRPGMLVPWDGGELFATLNAASQLHVIHADINAAQNLQRRFWGRCGEAIRIVCNQLSVDGSTRYEMAKAPKARLLGALQQLKNGDAPFHLTSIPNSQKPENSYVMTPTNAGKKYRAGPGEKSSGEEDELALDIVEQAEELAQGRKTFFRDPSGVFFAPDRWLPSEIYWSRIRRRIWQVTLERNSSGRQERAEMDEMPY

SEQ ID NO: 170 - protein, 1097 aa, unidentified
  source 1..1097: note = Description of Unknown: Cas12 sequence
MVTRALNLKLVVPRRPGELTKAEALWSTHDIVNRATSYYESQLLLCRQQDYQTRELTVSAGDQAPDLDALIANARDRNRYRGLEKPQVVREKLRNLYEAIVPPAIGKTGTAQAVGAFVSPLLDADSRGFTEIFDKIEALPNWVDGVRAEEPDALEAAADWLKSPQGKERLRPTGAPPTWIKLAKKKDAGWAAAFVADIDKKLKEVEGTPTLMQELRALGVMPLFPSFFASRIAGHKGAVSTWDRLALRLAVAHLLSWESWVELAAKEHAARVAKLEKFRDDNILGEIADAVEALRLYEKERTEELQQKAQLDAEEVRTTSRTIRGWVDLREKWLKTDASPDALISLVAAEQKRKSGKFGDPQLFRWLAKPENHFVWNKPDFDPPSLFASLRMIEGLVERSKETAWMTLPDARLHPRSSQWEPHGGGNLKTFRLEQGEGGSLSVTLPLLRKSGDDSYVEEEHAFSLAGSKQIPNASLDVRRNKYCLSYRTPTGEEAEAVVGSADLLLDWYFLQQRSEHRPEEGDIGPAFLKLALDITPIDPVWGEREKTPAIHHFKTASGKNTRHADGVAPGFRMLAVDLGIRTLATCSVFELKATAPAGRLSFPIAHLDLHAVHERSFTLTLDGEDPDRDAERWRENKSAELRRLRMGLTRYRNIRNMREDAPDEREVLLEDLQEKVQEHGWAFEEPLLRELAKHKDTPEPIWEAELTKALAQFRSDFGVIVGEWRRSNRARSTDSHAGKSMWAIDHLTNSRRFLMSWSLLSKPGQIRRLDRDKQGVFAKHLLDHLEGLKADRLKTGSDLIVQAARGFRRDKRGNWHKAYKPCHGILFEDLSRYRMRTDRPRRENSQLMKWAHRAVPKEVGMQAEVYGIRVEDTGAAFSSRFHAASHTPGIRMHPICQKDLENEWLLDEIEKQNSGVKRRELKLGQLVQLNGGELFACVTASGVKTLHADINAAQNLQRRFFTRHGDAFRIVARKVLVDEEEVWVPRSLGKRLLGALGSHGKLVPTGHESGSCRFEEITTRAWSKLSGEKLSDDRVGNEEDQIIASIEEEALERTGEVVVFFRDPSGQVLPRDLWYPSKTFWSIVKSTTLSKLKAAP

SEQ ID NO: 171 - protein, 1272 aa, unidentified
  source 1..1272: note = Description of Unknown: Cas12 sequence
MEDKQFLERYKEFIGLNSLSKTLRNSLIPVGSTLKHIQEYGILEEDSLRAQKREELKGIMDDYYRNYIEMHLRDVHDIDWNELFEALTEVKKNQTDDAKKCLEKIQEKKRKEIYQYLSDDAVFSEMFKEKMISGILPDFIRCNEEYSEEEKEEKLKTVALFHRFTSSFNDFFLNRKNVFTKEAIATAIGYRVVHENAEIFLENMVAFQNIQKSAESQISIIERKNEHYFMEWKLSHIFTADYYMMLMTQKAIEHYNEMCGVVNQHMKEYCQKEKKNWNLYRMKRLHKQILSNASTSFKIPEKYENDAEVYESVNSFLQNVMEKTVMERIAVLKNNTDNFDLSKIYITAPYYEKISNYLCGSWNTIADCLTHYYEQQIAGKGARKDQKVKAAVKADKWKSLSEIEQLLKEYARAEEVKRKPEEYIAEIENIVSLKEVHLLEYHPEVNLIENEKYATEIKDVLDNYMELFHWMKWFYIEEAVEKEVNFYGELDDLYEEIRDIVPLYNKVRNYVTQKPYSDTKIKLNFGTPTLANGWSKSKEYDYNAILLQKDGKYYMGIFNPVQKPEKEIIEGHSHPLEGNEYKKMVYYYLPSANKMLPKVLLSKKGMEIYQPSEYIINGYKERRHIKSEEKFDLQFCHDLIDYFKSGIERNPDWKVFGFHFSDTDTYQDISGFYREVEDQGYKIDWTYIKEADIDRLNEEGKLYLFQIYNKDFSEKSTGRENLHTMYLKNLFSEENIREQVLKLNGEAEIFFRKSSVKKPIIHKKGTMLVNRTYMEEMHGESVKKNIPEKEYQEIYNYMNHRWKGELSAEAKEYLKKAVCHETKKDIVKDYRYSVDKFFIHLPITINYRASGKEALNSVAQRYIAHQNDMHVIGIDRGERNLIYVSVINMQGEIIEQKSFNVVNKYNYKEKLKEREQNRDEARKNWKEIGQIKDLKEGYLSGVIHEIAKMMIKYHAIVAMEDLNYGFKRGRFKVERQVYQKFENMLIQKLNYLVFKDRSADEDGGVLRGYQLAYIPDSVKKLGRQCGMIFYVPAAFTSKIDPATGFVDIFNHKAYTTDQAKREFILSFDEICYDVERQLFRFTFDYANFATHNVTLARNNWTIYTNGTRTQKEFVNRRVRDKKEVFDPTEKMLKLLELEGVEYQSGANLLPKLEKISDPHLFHELQRIVRFTVQLRNSKNEENDVDYDHVISPVLNEEGKFFDSSKYENKEEKKESLLPVDADANGAYCIALKGLYIMQAIQKNWSEEKALSPDVLRLNNNDWFDYIQNKRYR

SEQ ID NO: 172 - protein, 767 aa, unidentified; identical to SEQ ID NO: 112
  source 1..767: note = Description of Unknown: Cas12 sequence

SEQ ID NO: 173 - protein, 1147 aa, unidentified; identical to SEQ ID NO: 4
  source 1..1147: note = Description of Unknown: Cas12 sequence

SEQ ID NO: 174 - protein, 687 aa, unidentified; identical to SEQ ID NO: 147
  source 1..687: note = Description of Unknown: Cas12 sequence
  VARIANT 156..527: note = MOD_RES - Any amino acid

SEQ ID NO: 175 - protein, 1212 aa, unidentified
  source 1..1212: note = Description of Unknown: Cas12 sequence
MIKKSNFWQFTGLYELSKTLRFELKPVWETLNILEKDWVSQKDKEVEENYNKIKVFFDSLHREFVKQSLENWYLELLENFYNSYIELNKNIENKKNKSLQKLFEKSSKELKKELVSFFEWKWNDWKQKYSFLKKWWIDVLNEKEVLDLMWEFYPKEKELFKKFDKFFTYFSNFKESRKNFYADDWRAWAIATRVIDENLITFIKNIEDFKKFKNNFSDFIENGFSDWKIKINWFTLEEKQVFDLDFYNNCLLQDWIDNYNKILWWFSEENWNKIQWINEKINLFKQNQNKTNSKDVKFPRFKLLYKQILSEKEKLIFVDEIENDEKLINFIKESKNNNLIKVEKAIEITENFIKNNETFELDKIYLSKISINTISNKFFSSWDYILKEGFDKWEIKEFISFEDLKNAFGKIKYENLEDIFKSNYITDYIAINWEDLYKNFLNIFLYEFKQNINEINFYNSELEKLFLEKFEKTETQVQIIKNYFDSVLSLYKMTKYFALEKGKKKIEDLETDNNFYNDFFVYYEDFEIWKDYNLVRNFITKKQVKTDKFKLNFENSQFLTGWDKDKEKERLWIILKKDEKYYLWILKNNKIFNSYNYESWDFYEKMSYKQLNNVYRQLPRFAFSKAKREVYWITPELEQIKEEFDIFQKNKEKWEKFDIEKLKKLINCYKKWFIKTYENEFDLEKIKNTDYLDLATFYDEIEQKTYKIDFNKISENFINAKVNSWELYLFQIYNKDFSETKKAWSKENIHTKYFKLLFDEKNLEKLVIKLSWWAEMFFREKTEKLKTKLDKSWKEVLEHRRYSKDKIMLHLSITLNANKWDSFWFNKMVNEYLNKNEDIKIIWIDRGEKHLAYYSVIDKNWKIEEIDTLNIIKSSDWKITNYLEKLEKIESSRKDSRVSWWEIENIKELKNGYISQVVNKLAELIIKYNAIIVFEDLNIWFKRWRQKIEKQIYQKLELALAKKLNYLTQKDKNDNEVLWNLKALQLVPKVNDYQDIANYKQSGIMFYTRANYTSTTCPCCWFRKNIYISNSDTKEKQKKDFEKIDIKFDGEKFIFSYEIIQDKKAKQKSNKTNFSVNSNFSRFKYNSKKMLVEEVNLNLELQNLFKDIDLKWDINKQILEKDSYFYKSLTYYFNLILQLRNSDSKNDIDYITCPSCNYHSKDWFQGLEYNADANWAYNIARKWIIMLDRIEKNFEKPDLYVSDIDWDNFTQK

SEQ ID NO: 176 - protein, 1212 aa, unidentified; identical to SEQ ID NO: 175
  source 1..1212: note = Description of Unknown: Cas12 sequence

SEQ ID NO: 177 - protein, 1108 aa, unidentified; identical to SEQ ID NO: 33
  source 1..1108: note = Description of Unknown: Cas12 sequence

SEQ ID NO: 178 - protein, 688 aa, unidentified
  source 1..688: note = Description of Unknown: Cas12 sequence
  VARIANT 276..564: note = MOD_RES - Any amino acid
MAESMYNQQNINQKSTETPTEEDALGIKNPLLTPTTLGQNFLSLQSISPLGSRSINLINWSLISPNQTLLQNFQDNENWQDSSISEFRPFSKNSLTENSPIIEPQSDKTLPSSVPIQLSSELPIQQPPETSFIDSESPIPQPPETPSSDSESPIGQTENIFPFQIDQKTRRNSVIQNSKSSISHFFQKKSEFPREKISQENVNKLTKKSAENQDISTTEESVINSIETNDLQTINPTDIPNPEIPVSTLQKQPDSTIDNIPQTESVSTINPTDIPXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXSKNNRIPQLITFLKLNQYQQLILQIFLTQKFSFQLSKNNRIPPLITFLKLNQYQQLIPRVLLHQKFLFQLFKNNRIPPLITFPKLNQYQQLIPRIFLTQKFPFQISRNNRIPQLITFPKLNQYQ

SEQ ID NO: 179 - protein, 1003 aa, unidentified
  source 1..1003: note = Description of Unknown: Cas13 sequence
MGAIKNKHIFAAYANLAIDGLIKTLNFIAKKLDTQKQLSSWDIKHVITLIDSIFDQNPQNNLEQVVEGYLPWIKPIIEMKTPKKGERQSDKLCIEYKTIITAFASLLNDVRNYYTHYYHDPICIYPGGYDIPSSLNCIYDSAINIIKERFQAEEKEMEHLRRYTRKKGRVVLKTEDDHFYYTLANNNDLSEKGYAFFISMFLERKYSYLFLKKLSGFKRGDSLQYRLTLEVFTALSTKPPVERLRTTKDTKQDRALDILNELSKIPIELYQTLEPKYREMYNETLQPTDAEDPYGLPDRSRIRFRSRFEAFALHFLDKQADFKEIGFYTYLGNYFHNGYQKTRVDRETKDRYINFQLAGFCKNIQDISAKKLSEALNVKSIDISTDSIPDINSFEPYLVQSTPHYIVNGNNIGIKVLPEGKDTYPTIDEKGAKMPIADFWLSKYELPAMLFYTYLRNNNIHKSHCPLSVKDIIERSIHKSTKQKHPEERSELMLRRVMKAIFWTDSKLNEVERIKSQKSAFGKRQHEILKAGRIAETLVRDMLWLQPSKNNGRDKVTEPNFQAIQVSLAYFGIRRNDLTEIFTRAGLINSSNPHPFLAQIGTNYTSLIEFYIAYLKERKVYFSRIQKKILQGKLNIQCHPLRDLQREPNKPQDKEEAIFLPRGLFNEAIINCLKKSKLKQLIESPTREKSPALNVSYLIQNYFRTYFEDQSQEFYAQPRNYRLFDKLSPNKGKSKSYLSLEQRIKKMEELRPSKIPVAEANKLLEKEDRLYRKNYNEICDNESIIRLYQIQDILLFMMTKEYLPSDLYNRINKYKLENVKGILNERVSYLIDLNPLKIQGEDIKIKDYGKLFYIHHDTRISSLNKVLSKVKRNNSISSSVKIQPYENYKRECLDFEEAQIQIIPIIHSFEIAMVSMFPDLKKATPGNYYDFNELITEYEKRTKQKIGSSFLIKTRNMFLHDKYEAECIKEISDDFVYAKKIIAEFKMKIENIKLEDLSNDSSA

SEQ ID NO: 180 - protein, 986 aa, unidentified
  source 1..986: note = Description of Unknown: Cas13 sequence
MARDNFLNTIKLLADKMKLGAISGFGKDGNEVNDINKLFGDKNTYANIENVVEFYFPWIKALEGRFSLDKGDRNLNDMKMFYKSVLTAFFTAVDSLRNKYTHYSHKDLNIREIKIECTLGGKDYCIGLLNALDCIYDSAVNLLKLRFMVGEDEVAHLRRCKAVNKKVVVRTEKDGFYYRLSDNGGVTEKGVIFIASMFLNRKYGFLFLKQLEGFKRSDEKRYRLTLEAFLAFSNIKPVDRLKSDKLDRASLGLDMLNELTKIPKELSETLSVDCLYKYLASDGEDDLRSRIRYQDRFVPLALEFISQSDEFKDFRFYTYVGNYVYKGYIKRLIDGTDKERYLSDRLCGFYKSVNDASSDAIAQKYGVEIKDSNEPDYMLPDSFRPHVLRATPHFVINNNNIGIKICGNDCLPIVNGKGVESPEPDYWLSIYELPAMLFYAYLREKNGKRFKDYKSIRELIEGVEKKADEKNDRDKGALMARHIDKEIIWTQTKLDEVKRLEEKKVAAYGKKGRVVLKSGRMADLLAHDMVRLQPATKGSDKITGVNFQALQVSLAYFKRDILADVFSRAMLTTGNHRHPFLYRIDVSHCSSLRDFYVAYLGERRKYFEDVAKKIAKNKLNTPCHILRRLQREGSGEEAGKDVKPKFLPRGIFTDSIKNCLEQSKLNIYIRNARNDVKPAINAAYLILMYYKEIEKGEFQGFYGEKRRYDILEEGKPLDLAERKKALASIKPAKIDVSEANMPMSKEEHLMRKRYHAVCNNESAIRMYQVQDILLLLMAKDIFKKALSEGVMSKKIGLENLNGIFDAPVNFVKNFDNIKLTATGIKIKNYGKVCRLGTDFKFNSLIKAFHKVYSKSVEMDYSDYLKEEEEFEKYRLNMVKLCREVERGITEDLHLSLDGKSHLSFNDDVIKPYNDKYNVFNGDDLTFFINARNMFMHGDYKYECVKYVVSEHFKGSLNDVSFAKETYGHFCNLLESMRKKTGLRIDI

SEQ ID NO: 181 - protein, 1009 aa, unidentified
  source 1..1009: note = Description of Unknown: Cas13 sequence
MKKKISLKEQRNTKKAENKLKYQKAQAERAAAAQQTAAGAESEENPCFDVVKDTKRKALNPLHVEIEAPSAKKSSVKANGLKSLLLTDGKTVMTSFGRGSEANVEKRFDETGTKTFDRDPELFSAKPLETGYRIQRFNASPKDAGLAYRPAGVRPDQIGAKAALEKRYFGKETPGDNIHVQIAYQIQDIEKLLAVYISNIIYAVNNVTGVSAMKDSKGRPVDLLGDYGILGEEGLTKRLQRIPEQADEEAKALQAFLCSERLSYFGKEFCLVRNSPKQPDKEEKRQYKLMRVLCLLGELRQFLVHGKKKEKEFAWLYRLDRQLSQEYRKLLGEFYDAQVDKVNKSFLTNSTVNLEVLFRALKTGTDPERKTVTQEYYQFTVRKEDGNLGFSLKTLREILLSAYKHEVRDKEYDSIRHKLYQLFSFALYHYYKTGVGAERREAFVAKLRAVMTAEAKQRAYADEAAEIWNDEGSGIRAAFLEILEAVDFGSAVKGIKARSSVAGDKRFAEWLEEVRIRPEGVSCFTKLMYLLTRFLDGKEINELLTGLINKLENIQSFLDVMQQEHAETGLSDAFSFFEYSGEIAAELRMTRSFARMAAADPEAKRFMVVDGAKLLGFNPKDTESEDEGIIRAIYGDACAEYLQFSEEEKEAFYVQEGLYGKEREKFSPYAYFHTDTSLRNFIAKNVVESARFRYVIRYVSPEIARKYARQEALVRFALHRVPLLQLRRYYQSCCGPKKDPDAAECVDFLAGVVNRVDFANFTDVRTGDSSKSEQEKKQKYQAIVGLYLTVVYWIVKNLVNVNSRYVMAFHILERDTVLLEGKRLFVGGMKAEDPFLLTDGYVSRQDAYVRKRIGENKRANRHGLNCVLENRNALGSDPASTDAAASLIWSYRNAAAHLTAVAAAQEYVSELREIHSYFEVYHYAMQRYLKSGAEFAELVSKNGPASGKIAAWANAVDRCHSFCKDWLWLLNVPFAYNPARYKNLSIANLFDKNEAAPVTEDASEQKEDE

SEQ ID NO: 182 - protein, 1003 aa, unidentified
  source 1..1003: note = Description of Unknown: Cas13 sequence
MGAIENKHIFAAYANLAIDGLIKTLNFIAKKLDTQKQLSSWDIKHVITLIDSIFDQNPQNNLEQVVEGYLPWIKPIIEMKTPKKGERQSDKLCIEYKTIITAFASLLNDVRNYYTHYYHDPICIYPGGYDIPSSLNCIYDSAINIIKERFQAEEKEMEHLRRYTRKKGRVVLKTEDDHFYYTLANNNDLSEKGYAFFISMFLERKYSYLFLKKLSGFKRGDSLQYRLTLEVFTALSTKPPVERLRTTKDTKQDRALDILNELSKIPIELYQTLEPKYREMYNETLQPTDAEDPYGLPDRSRIRFRSRFEAFALHFLDKQADFKEIGFYTYLGNYFHNGYQKTRVDRETKDRYINFQLAGFCKNIQDISAKKLSEALNVKSIDISTDSIPDINSFEPYLVQSTPHYIVNGNNIGIKVLPEGKDTYPTIDEKGAKMPIADFWLSKYELPAMLFYTYLRNNNIHKSHCPLSVKDIIERSIHKSTKQKHPEERSELMLRRVMKAIFWTDSKLNEVERIKSQKSAFGKRQHEILKAGRIAETLVRDMLWLQPSKNNGRDKVTEPNFQAIQVSLAYFGIRRNDLTEIFTRAGLINSSNPHPFLAQIGTNYTSLIEFYIAYLKERKVYFSRIQKKILQGKLNIQCHPLRDLQREPNKPQEKEEAIFLPRGLFNEAIINCLKKSKLKHLIESPTREKSPALNVSYLIQNYFRAYFEDQSQEFYAQPRNYRLFDNLSPNKGKSKSYLSLEQRIKKMEELRPSKIPVAEANKLLEKEDRLYRKNYNEICDNESIIRLYQIQDILLFMMTKEYLPSDLYNRINKYKLENVKGILNERVSYLIDLNPLKIQGEDIKIKDYGKLFYIHHDTRISSLNKVLSKVKRNNSISSSVKIQPYENYKRECLDFEEAQIQIIPIIHSFEIAMVSMFPDLKKATPGNYYDFNELITEYEKRTKQKIDSSFLIKTRNMFLHDKYEAECIKEISDDFVYAKKIIAEFKMKIENIKLEDFSNDSSA

SEQ ID NO: 183 - protein, 985 aa, unidentified
  source 1..985: note = Description of Unknown: Cas13 sequence
MGAIENKHIFAAYANLAIDGLIKTLNFIAKKLDTQKQLSSWDIKHVITLIDSIFDQNPQNNLEQVVEGYLPWIKPIIEMKTPKKGERQSDKLCIEYKTIITAFASLLNDVRNYYTHYYHDPICIYPRGYDIPSSLNCIYDSAINIIKERFQAEEKEMEHLRNYTLVNNNGLSEKGYAFFISKFLERKYSYLFLKKLSGFKRGDSLQYRLTLEVFTALSTKPPVERLRTTKDTKQDRALDILNELSRIPIELYQTLEPKYREMYNETLQPTDAEDPYGLPDRSRIRFRSRFEAFALHFLDKQADFKEIGFYTYLGNYFHNGYQKTRVDRETKDRYINFQLAGFCKNIQDISAKKLSEALNVKSIDISTDSIPDINSFEPYLVQSTPHYIVNGNNIGIKVLPEGKDTYPTIDEKGAKMPIADFWLSKYELPAMLFYTYLRNNNIHKSHCPLSVKDIIERSIHKSTKQKHPEERSELMLRRVMKAIFWTDSKLNEVERIKSQKSAFGKRQHEILKAGRIAETLVRDMLWLQPSKNNGRDKVTEPNFQAIQVSLAYFGIRRNDLTEIFTRAGLINSSNPHPFLAQIGTNYTSLIEFYIAYLKERKVYFSRIQKKILQGKLNIQCHPLRDLQREPNKPQEKEEAIFLPRGLFNEAIINCLKKSKLKHLIESPTREKSPALNVSYLIHNYFRAYFEDQSQEFYAQPRNYRLFDKLSPNKGKSKSYLSLEQRIKKMEELRPSKIPVAEANKLLEKEDRLYRKNYNEICDNESIIRLYQIQDILLFMMTKEYLPSDLYNRINKYKLENVKGILNERVSYLIDLNPLKIQGEDIKIKDYGKLFYIHHDTRISSLNKVLSKVKRNNSISSSVKIQPYENYKRECLDFEEAQIQIIPIIHSFEIAMVSMFPDLKKATPGNYYDFNELITEYEKRTKQKIDSSFLIKTRNMFLHDKYEAECIKEISDDFVYAKKIIAEFKMKIENIKLEDLSNDSSA

SEQ ID NO: 184 - protein, 1082 aa, unidentified
  source 1..1082: note = Description of Unknown: Cas13 sequence
CILDFFTQDKAIAEYQLGVEFLQKNLPVIRYLYLPTSHKRFENVPKNQLISEQRNYFKNSLKVLKNLIRDYRNFYTHHFHKPIPVFPETYKLLDDLFLAVANDVKKHRMKTDASKQLLKKGLIEELAQLEKLKLEDLKKLKREGKKVNLNDKEAITNAILNDSFSHLLPKENTISKYYSAVPTEDIDTENGVTISESGIIFLLGLFLTKKQSEDLRSRVKGFKAKLIVNPENPINKKNNSLKYMATHWVFGYLGFKGLKNRFTTTFTKDTLLAQIVDELSKVPDELYQVLPEELKNEFLEDMNEYLKEENSESLDKATVIHPVIRKRYENKFAYFALRFLDEFVDFPTLRFQLHLGNYVHDKREKPIEGTKYVTERIVKEKIKAFAKLSEAAQLKQKYFEEKENHQSIGLQLYPNPSYNFVGNNIPIHLNLNEHFFPKEVKIVAGRLKKRNSSYKSDHPEEYKVRTDNKIKPDAILQDLGKPEKLAPVAMLSLNELPALLHLVLTKKTPEEIEIIIAQKIAERYNVLTNYKAGDDISKGQITKNLLKAKQKKEVNLDKLQLAIEKEIAVTNDKLQTIALHIKERNDPKQKRKYVFTNKEIGLQVTWLANDLKRFMPKGSRQNWRGQHHSQLQKSLAFYDIQPKEPLSLLEEVWDFKNEAYLWNNGIRRSFDKRDFISFYTSYLNNRKETFQRFKDQLNGIRSNKKILDKFIKQQHLWNLFHKRLYVIDTIEEQVEKLLVKPMQFPKGVFDHKPTYIKGKSIQENPECFADWYVAWNQHTDYQKFYSWDRDYKSAYLSGEQEKTEKRFIRVQGSKINKVKQQDVLLAKMASIIFNELYLPEDAEHLDLNLSDIYKTQTERKAEIEAALIQSHKTTGDNSANIIKSTSAWTLTVPYCSKNIYEPQVKLKELGKFKKFIASQKVQTLFEYKPQKIWNKTELEEVLELKANSYEVIRRDYLLKSIQEFEKYMIKKLPTLIDTNEHPNFNKYLTTFLKSLELVSEEDAKWLISKKDFDTTPIDELKKQSKIMEKAFLLVMIRNKFSHNQLPRKIYYDEIYKNVPNAVSINFNELFLEYTNQTILEFK

SEQ ID NO: 185 - protein, 708 aa, unidentified
  source 1..708: note = Description of Unknown: Cas13 sequence
NRRDMEASPGRLARYIVLKTLYERAFPRWLEAREAETLNGWIGRAADRATVAARHINKDENAAARMAGLVRLADGKGIADFTDRLAAETASEYRVQRGYDSDPAVARKQAKHIEDLRCDVVGQAFEAYLADVARKLAWTMNDPPGGPLPDEKKASLETAAPPAEDAGAEAEEDWLARLYFLLHMLPVEEVGGLRHQLRKWSVLEREPDSVLEREPDADVEAIERILGLYLDMHDAKFEGGDGVAGAEALTDLFTSPAQFRRVCPEDGAGNGGHVPWRGLREMLRFGGGEPRLMWAFRKWPIGDGMVDGLNALEATVAEAHKQREDLHAKWAGKKGFSRKDKDEYRAALETVVVHRHLAAHVQLVNHARLHRLAMAVLARLADYAGLWERDLYFTTLALIRLENGKPEDVFRSRELEWLREGRIVDALKELKKNADGSEPAVGTALQRLFGKGILTGGGTVSVRRDLLHFNMLQRKANEPFNLTTAVNDTRKLMAYDRKLKNAVSRSIIELLAREGLDLAWEMKDHQLAGAVLKTRQAVHLGGAKVGGGPITEDLHGPEFTAMAAALFGGEARAATDAAEAATGRPDRRERKSGGASPRPARPAKTLPAPTPRLLPAAGERVEGVLVEEKTRKGGWKAAVEIGGPRIVGDIFNSGEVPSDAEPGLEAEFVVRVANPANASFMWLSPDVEERLKKAAAPRRGGRPAGRQR

SEQ ID NO: 186 - protein, 1003 aa, unidentified; identical to SEQ ID NO: 182
  source 1..1003: note = Description of Unknown: Cas13 sequence

SEQ ID NO: 187 - protein, 985 aa, unidentified; identical to SEQ ID NO: 183
  source 1..985: note = Description of Unknown: Cas13 sequence

SEQ ID NO: 188 - protein, 1003 aa, unidentified; identical to SEQ ID NO: 182
  source 1..1003: note = Description of Unknown: Cas13 sequence

SEQ ID NO: 189 - protein, 985 aa, unidentified; identical to SEQ ID NO: 183
  source 1..985: note = Description of Unknown: Cas13 sequence

SEQ ID NO: 190 - protein, 1000 aa, unidentified
  source 1..1000: note = Description of Unknown: Cas13 sequence
MEEANRYIYGAYFNMARDNFLNTIKLLADKMKLGATSGFGKDGNEVNDINELFGDKNTYANIENVVEFYFPWIKALEGRFSLDKGDRNLNDMKMFYKSVLTAFFTAVDSLRNKYTHYSHKDLNIREIKIECTLGGKDYCIGLLNALDCIYDSAVNLLKLRFMAGEYEVAHLRRCKAVNKKVVVRTEKDGFYYRLSDNGGVTEKGVIFIASMFLNRKYGFLFLKQLEGFKRSDEKRYRLTLETFLAFSNIKPVDRLKSDKLDRASLGLDMLNELTKIPRELSETLSVDCLYKYLTSDGEDDLRSRIRYQDRFVPLALEFISQSDEFKDFRFYTYVGNYVYKGYIKRLIDGTDKERYLSDRLCGFYKSVNDASSDAIAQKYGVEIKDSNEPDYMLPDSFRPHVLRATPHFVINNNNIGIKICGNDCLPIVNGKGVESPEPDYWLSIYELPAMLFYAYLREKNGKLLKDYKSIRELIEDVEKKADEKNDRDKGALMARHIDKEIIWTQTKLDEVKRLEEKKVAAYGKKGRVVLKSGRMADLLAHDMVRLQPATKGSDKITGANFQALQVSLAYFKRDILADVFSRAMLTTGNHRHPFLYRIDVSHCSSLRDFYVAYLGERRKYFEDVAKKIAKNKLNTPCHILRRLQREGSGEEAGKDVKPKFLPRGIFTGSIKSCLEKSALNINIRNARNDVKPAINAAYLILMYYKEIEKGEFQGFYGEKRRYDILEEGKPLDLDERKKALASIKPAKIDVSEANMPMSKEEHLMRKRYHAVCNNESAIRMYQVQDILLLLMAKDIFKKALSEGVMSKKIGLENLNGIFDAPVNFVKNFDNIKLTATGIKIKDYGKVCRLGTDFKFNSLVKAFHKVYSKSVEMDYSDYLKEEEEFEKYRLNMVKLCREVERGITEDLHLSLDGKSHLGFNDDVIKPYNDKYNVFNGGDLTFFINARNMFMHGDYKYECVKYVVSEHFKGSLNDVSFAKETYGHFCNLLESMRKKTGLRIDI

SEQ ID NO: 191 - protein, 1003 aa, unidentified; identical to SEQ ID NO: 179
  source 1..1003: note = Description of Unknown: Cas13 sequence

SEQ ID NO: 192 - protein, 1003 aa, unidentified; identical to SEQ ID NO: 182
  source 1..1003: note = Description of Unknown: Cas13 sequence

SEQ ID NO: 193 - protein, 1003 aa, unidentified
  source 1..1003: note = Description of Unknown: Cas13 sequence
MGAIENKHIFAAYANLAIDGLIKTLNFIAKKLDTQKQLSSWDIKHVITLIDSIFDQNPQNNLEQVVEGYLPWIKPIIEMKTPKKGERQSDKLCIEYKTIITAFASLLNDVRNYYTHYYHDPICIYPGGYDIPSSLNCIYDSAINIIKERFQAEEKEIEHLRRYTRKKGRVVLKTEDDHFYYTLVNNNDLSEKGYAFFISMFLERKYSYLFLKKLSGFKRGDSLQYRLTLEVFTALSTKPPVERLRTTKDTKQDRALDILNELSRIPIELYQTLEPKYREMYNETLQPTDAEDPYGLPDRSRIRFRSRFEAFALHFLDKQADFKEIGFYTYLGNYFHNGYQKTRVDRETKDRYINFQLAGFCKNIQDISAKKLSEALNVKSIDISTDSIPDINSFEPYLVQSTPHYIVNGNNIGIKVLPEGKDTYPTIDEKGAKMPIADFWLSKYELPAMLFYTYLRNNNIHKSHCPLSVKDIIERSIHKSTKQKHPEERSELMLRRVMKAIFWTDSKLNEVERIKSQKSAFGKRQHEILKAGRIAETLVRDMLWLQPSKNNGRDKVTEPNFQAIQVSLAYFGIRRNDLTEIFTRAGLINSSNPHPFLAQIGTNYTSLIEFYIAYLKERKVYFSRIQKKILQGKLNIQCHPLRDLQREPNKPQDKEEAIFLPRGLFNEAIINCLKKSKLKQLIESPTREKSPALNVSYLIQNYFRTYFEDQSQEFYAQPRNYRLFDKLSPNKGKSKSYLSLEQRIKKMEELRPSKIPVAEANKLLEKEDRLYRKNYNEICDNESIIRLYQIQDILLFMMTKEYLPSDLYNRINKYKLENVKGILNERVSYLIDLNPLKIQGEDIKIKDYGKLFYIHHDTRISSLNKVLSKVKRNNSISSSVKIQPYENYKRECLDFEEAQIQIIPIIHSFEIAMVSMFPDLKKATPGNYYDFNELITEYEKRTKQKIDSSFLIKTRNMFLHDKYEAECIKEISDDFVYAKKIIAEFKMKIENIKLEDLSNDSSA

SEQ ID NO: 194 - protein, 1003 aa, unidentified; identical to SEQ ID NO: 182
  source 1..1003: note = Description of Unknown: Cas13 sequence

SEQ ID NO: 195 - protein, 1003 aa, unidentified
  source 1..1003: note = Description of Unknown: Cas13 sequence
MGAIENKHIFAAYANLAIDGLIKTLNFIAKKLDTQKQLSSWDIKHVITLIDSIFDQNPQNNLEQVVEGYLPWIKPIIEMKTPKKGERQSDKLCIEYKTIITAFASLLNDVRNYYTHYYHDPICIYPGGYDIPSSLNCIYDSAINIIKERFQAEEKEMEHLRRYTRKKGRVVLKTEDDHFYYTLANNNDLSEKGYAFFISMFLERKYSYLFLKKLSGFKRGDSLQYRLTLEVFTALSTKPPVERLRTTKDTKQDRALDILNELSKIPIELYQTLEPKYREMYNETLQPTDAEDPYGLPDRSRIRFRSRFETFALHFLDKQPDFKEIGFYTYLGNYFHNGYQKTRVDRETKDRYINFQLAGFCKNIQDISAKKLSEALNVKSIDISTDSIPDINSFEPYLVQSMPHYIVNGNNIGIKVLPEGKDTYPTIDEKGAKMPIADFWLSKYELPAMLFYTYLRNNNIHKSHCPLSVKDIIERSIHKSTKQKHPEERSELMLRRVMKAIFWTDSKLNEVERIKSQKSAFGKRQHEILKAGRIAEMLVRDMLWLQPSKNNGRDKVTEPNFQAIQVSLAYFGIRRNDLTEIFTRAGLINSSNPHPFLAQIGTNYTSLIEFYIAYLKERKVYFSRIQKKILQGKLNIQCHPLRDLQREPNKPQEKEEAIFLPRGLFNEAIINCLKKSKLKQLIESPTREKSPALNVSYLIQNYFRAYFEDQSQEFYAQPRNYRLFDKLSPNKGKSKSYLSLEQRIKKMEELRPSKIPVAEANKLLEKEDRLYRKNYNEICDNESIIRLYQIQDILLFMMTKEYLPSDLYNRINKYKLENVKGILNERVSYLIDLNPLKIQGEDIKIKDYGKLFYIHHDTRISSLNKVLSKVKRNNSISSSVKIQPYENYKRECLDFEEAQIQIIPIIHSFEIAMVSMFPDLKKATPGNYYDFNELITEYEKRTKQKIDSSFLIKTRNMFLHDKYEAECIKEISDDFVYAKKIIAEFKMKIENIKLEDLSNDSSA

SEQ ID NO: 196 - protein, 985 aa, unidentified; identical to SEQ ID NO: 183
  source 1..985: note = Description of Unknown: Cas13 sequence

SEQ ID NO: 197 - protein, 1003 aa, unidentified; identical to SEQ ID NO: 193
  source 1..1003: note = Description of Unknown: Cas13 sequence

SEQ ID NO: 198 - protein, 1003 aa, unidentified
  source 1..1003: note = Description of Unknown: Cas13 sequence
MGAIENKHIFAAYANLAIDGLIKTLNFIAKKLDTQKQLSSWDIKHVITLIDSIFDQNPQNNLEQVVEGYLPWIKPIIEMKTPKKGERQSDKLCIEYKTIITAFASLLNDVRNYYTHYYHDPICIYPGGYDIPSSLNCIYDSAINIIKERFQAEEKEMEHLRRYTRKKGRVVLKTEDDHFYYTLANNNDLSEKGYAFFISMFLERKYSYLFLKKLSGFKRGDSLQYRLTLEVFTALSTKPPVERLRTTKDTKQDRALDILNELSRIPIELYQTLEPKYREMYNETLQPTDAEDPYGLPDRSRIRFRSRFEAFALHFLDKQADFKEIGFYTYLGNYFHNGYQKTRVDRETKDRYINFQLAGFCKNIQDISAKKLSEALNVKSIDISTDSIPDINSFEPYLVQSTPHYIVNGNNIGIKVLPEGKDTYPTIDEKGAKMPIADFWLSKYELPAMLFYTYLRNNNIHKSHCPLSVKDIIERSIHKSTKQKHPEERSELMLRRVMKAIFWTDSKLNEVERIKSQKSAFGKRQHEILKAGRIAETLVRDMLWLQPSKNNGRDKVTEPNFQAIQVSLAYFGIRRNDLTEIFTRAGLINSSNPHPFLAQIGTNYTSLIEFYIAYLKERKVYFSRIQKKILQGKLNIQCHPLRDLQREPNKPQEKEEAIFLPRGLFNEAIINCLKKSKLKQLIESPTREKSPALNVSYLILNYFRTYFEDQSQEFYAQPRNYRLFDKLSPNKGKSKSYLSLEQRIKKMEELRPSKIPVAEANKLLEKEDRLYRKNYNEICDNESIIRLYQIQDILLFMMTKEYLPSDLYNRINKYKLENVKGILNERVSYLIDLNPLKIQGEDIKIKDYGKLFYIHHDTRINSLNKVLSKVKRNNSISSSVKIQPYENYKRECLDFEEAQIQIIPIIHSFEIAMVSMFPDLKKATPGNYYDFNELITEYEKRTKQKIDSSFLIKTRNMFLHDKYEAECIKEISDDFVYAKKIIAEFKMKIENIKLEDFSNDSSA

SEQ ID NO: 199 - protein, 1003 aa, unidentified
  source 1..1003: note = Description of Unknown: Cas13 sequence
MGAIENKHIFAAYANLAIDGLIKTLNFIAKKLDTQKQLSSWDIKHVITLIDSIFDQNPQNNLEQVVEGYLPWIKPIIEMKTPKKGERQSDKLCIEYKTIITAFASLLNDVRNYYTHYYHDPICIYPGGYDIPSSLNCIYDSAINIIKERFQAEEKEMEHLRRYTRKKGRVVLKTEDDHFYYTLANNNDLSEKGYAFFISMFLERKYSYLFLKKLSGFKRGDSLQYRLTLEVFTALSTKPPVERLRTTKDTKQDRALDILNELSKIPIELYQTLEPKYREMYNETLQPTDAEDPYGLPDRSRIRFRSRFEAFALHFLDKQADFKEIGFYTYLGNYFHNGYQKTRVDRETKDRYINFQLAGFCKNIQDISAKKLSEALNVKSIDISTDSIPDINSFEPYLVQSTPHYIVNGNNIGIKVLPEGKDTYPTIDEKGAKMPIADFWLSKYELPAMLFYTYLRNNNIHKSHCPLSVKDIIERSIHKSTKQKHPEERSELMLRRVMKAIFWTDSKLNEVERIKSQKSAFGKRQHEILKAGRIAETLVRDMLWLQPSKNNGRDKVTEPNFQAIQVSLAYFGIRRNDLTEIFTRAGLINSSNPHPFLAQIGTNYTSLIEFYIAYLKERKVYFSRIQKKILQGKLNIQCHPLRDLQREPNKPQEKEEAIFLPRGLFNEAIINCLKKSKLKHLIESPTREKSPALNVSYLIQNYFRAYFEDQSQEFYAQPRNYRLFDKLSPNKGKSKSYLSLEQRIKKMEELRPSKIPVAEANKLLEKEDRLYRKNYNEICDNESIIRLYQIQDILLFMMTKEYLPSDLYNRINKYKLENVKGILNERVSYLIDLNPLKIQGEDIKIKDYGKLFYIHHDTRISSLNKVLSKVKRNNSISSSVKIQPYENYKRECLDFEEAQIQIIPIIHSFEIAMVSMFPDLKKATPGNYYDFNELITEYEKRTKQKIDSSFLIKTRNMFLHDKYEAECIKEISDDFVYAKKIIAEFKMKIENIKLEDLSNDSSA

SEQ ID NO: 200 - protein, 1003 aa, unidentified
  source 1..1003: note = Description of Unknown: Cas13 sequence
MGAIENKHIFAAYANLAIDGLIKTLNFIAKKLDTQKQLSSWDIKHVITLIDSIFDQNPQNNLEQVVEGYLPWIKPIIEMKTPKKGERQSDKLCIEYKTIITAFASLLNDVRNYYTHYYHDPICIYPGGYDIPSSLNCIYDSAINIIKERFQAEEKEMEHLRRYTRKKGRVVLKTEDDHFYYTLANNNDLSKKGYAFFISMFLERKYSYLFLKKLSGFKRGDSLQYRLTLEVFTALSTKPPVERLRTTKDTKQDRALDILNELSRIPIELYQTLEPKYREMYNETLQPTDAEDPYGLPDRSRIRFRSRFEAFALHFLDKQADFKEIGFYTYLGNYFHNGYQKTRVDRETKDRYINFQLAGFCKNIQDISAKKLSEALNVKSIDISTDSIPDINSFEPYLVQSTPHYIVNGNNIGIKVLPEGKDTYPTIDEKGAKMPIADFWLSKYELPAMLFYTYLRNNNIHKSHCPLSVKDIIERSIHKSTKQKHPEERSELMLRRVMKAIFWTDSKLNEVERIKSQKSAFGKRQHEILKAGRIAETLVRDMLWLQPSKNNGRDKVTEPNFQAIQVSLAYFGIRRNDLTEIFTRAGLINSSNPHPFLAQIGTNYTSLIEFYIAYLKERKVYFSRIQKKILQGKLNIQCHPLRDLQREPNKPQEKEEAIFLPRGLFNEAIINCLKKSKLKQLIESPTREKSPALNVSYLILNYFRTYFEDQSQEFYAQPRNYRLFDKLSPNKGKSKSYLSLEQRIKKMEELRPSKIPVAEANKLLEKEDRLYRKNYNEICDNESIIRLYQIQDILLFMMTKEYLPSDLYNRINKYKLENVKGILNERVSYLIDLNPLKIQGEDIKIKDYGKLFYIHHDTRINSLNKVLSKVKRNNSISSSVKIQPYENYKRECLDFEEAQIQIIPIIHSFEIAMVSMFPDLKKATPGNYYDFNELITEYEKRTKQKIDSSFLIKTRNMFLHDKYEAECIKEISDDFVYAKKIIAEFKMKIENIKLEDFSNDSSA

SEQ ID NO: 201 - protein, 1150 aa, unidentified
  source 1..1150: note = Description of Unknown: Cas13 sequence
MKHVFAAYFSDARLNIMASLNDVREKSGLKRYKNEAENVQNFEGIFPKNIASDIRDKRITLLRIRFPFIETIVDPGRGKENPASKYGELSWLFQLVNDMRNVFIHSTGSEEEIDYQHHKKIFNALRKVYDCGLRTVKSRFQLENDTTMPLLRCDNRGRPKPFNKFSLALCTSPERNGNKKQSDVLHDFGRVLLCSLFLEKRQISGLVSHFWDKNGYGTDWNDSEQTIIRELLYVNRIRLPSQRLRTDSTLTSVTLDTISELARCPRPLFELLDADGQENFRVGRSPKNPQNRNDDPSYLLLRGHQSRFIPLAMRHLDFDSKCKLRFAVDLGQYYHSVRLKPAESFIDGNPGIRRLGQKIIAFGRLKDFEDAEKPEIWKKLEENGQKFAEEEEELLKRASITGSPEELKPYIIKTFPHYHLYRDKIGFCIDWEEKKDQKVKYPNLGVRGEDSKSTDSGGIKKRENRQLSRHQFWISPNKIIDLAFYHYLQTEIKQEITASVKTGRKKCPYPSVENILKEYYEGMVVLIKELKEQGPLPPWTEIPQISERRAECHRWINKEIIKAENFTISLADLPKAIRRHLKVLDSRETSVSDIIKRTKTMIEETCKKQKEIEYLLKYPKKRGKKGFRPIKHGNIADFLTEDLLRFQPKDSSKKNGGKLTSKNYQILQKAIAYYDKPYCIVDLLKKSGLLEGEFKHPFLCKIITEENHEQYSTLIDFYQKYLEERKAFLEGFIDTFVAGSSIPGWLRLRKPSTFESWLDQQLDEDEKLCQPLPVPKSLFYQMLLKMTAEKLDLTPEMLCKKGTQRFIRDSQEVEVKPSVSWFIRQYMEHRDDTAQEMYMFKRRHELFDFFHDKKKTTKKLHEEKTCCLSEKVRQEELESVHKGIEKLKKLLRTYKRSEKKIRHFSTMDMVLYLLAKKNFEKLILCDETSGPDWSLKTLETKILSTKIKYELNVPGTDRTIVHRACAIKKTGELRLLVRDRRLPSLLDYYPKTEKSIDQEEIKVELADYSCKRIEAMKLISDLEQKIEESCNRGFCKPVPDKLKKIFGAKKHGTLLYALYHRFHEEKDKSVDEQGFNKDCFAHARQIRNAFAHNEYPVSEQFNQIVRQVNETSQPHNHNSPVNHPKIADQLVKELDKLYKPWRNFLKRIAG

SEQ ID NO: 202 - protein, 985 aa, unidentified; identical to SEQ ID NO: 183
  source 1..985: note = Description of Unknown: Cas13 sequence

SEQ ID NO: 203 - protein, 985 aa, unidentified; identical to SEQ ID NO: 183
  source 1..985: note = Description of Unknown: Cas13 sequence

SEQ ID NO: 204 - protein, 1003 aa, unidentified; identical to SEQ ID NO: 182
  source 1..1003: note = Description of Unknown: Cas13 sequence

SEQ ID NO: 205 - protein, 985 aa, unidentified; identical to SEQ ID NO: 183
  source 1..985: note = Description of Unknown: Cas13 sequence

SEQ ID NO: 206 - protein, 1003 aa, unidentified; identical to SEQ ID NO: 179
  source 1..1003: note = Description of Unknown: Cas13 sequence

SEQ ID NO: 207 - protein, 1003 aa, unidentified; identical to SEQ ID NO: 200
  source 1..1003: note = Description of Unknown: Cas13 sequence

SEQ ID NO: 208 - protein, 985 aa, unidentified
  source 1..985: note = Description of Unknown: Cas13 sequence
MGAIENKHIFAAYANLAIDGLIKTLNFIAKKLDTQKQLSSWDIKHVITLIDSIFDQNPQNNLEQVVEGYLPWIKPIIEMKTPKKGERQSDKLCIEYKTIITAFASLLNDVRNYYTHYYHDPICIYPGGYDIPSSLNCIYDSAINIIKERFQAEEKEMKHLRNYTLVNNNGLSEKGYAFFISKFLERKYSYLFLKKLSGFKRGDSLQYRLTLEVFTALSTKPPVERLRTTKDTKQDRALDILNELSKIPIELYQTLEPKYREMYNETLQPTDAEDPYGLPDRSRIRFRSRFEAFALHFLDKQADFKEIGFYTYLGNYFHNGYQKTRVDRETKDRYINFQLAGFCKNIQDISAKKLSEALNVKSIDISTDSIPDINSFEPYLVQSTPHYIVNGNNIGIKVLPEGKDTYPTIDEKGAKMPIADFWLSKYELPAMLFYTYLRNNNIHKSHCPLSVKDIIERSIHKSTKQKHPEERSELMLRRVMKAIFWTDSKLNEVERIKSQKSAFGKRQHEILKAGRIAETLVRDMLWLQPSKNNGRDKVTEPNFQAIQVSLAYFGIRRNDLTEIFTRAGLINSSNPHPFLAQIGTNYTSLIEFYIAYLKERKVYFSRIQKKILQGKLNIQCHPLRDLQREPNKPQEKEEAIFLPRGLFNEAIINCLKKSKLKQLIESPTREKSPALNVSYLIQNYFRTYFEDQSQEFYAQPRNYRLFDKLSPNKGKSKSYLSLEQRIKKMEELRPSKIPVAEANKLLEKEDRLYRKNYNEICDNESIIRLYQIQDILLFMMTKEYLPSDLYNRINKYKLENVKGILNERVSYLIDLNPLKIQGEDIKIKDYGKLFYIHHDTRINSLNKVLSKVKRNNSISSSVKIQPYENYKRECLDFEEAQIQIIPIIHSFEIAMVSMFPDLKKATPGNYYDFNELITEYEKRTKQKIDSSFLIKTRNMFLHDKYEAECIKEISDDLVYAKKIIAEFKMKIENIKLEDLSNDSSA

SEQ ID NO: 209 - protein, 1003 aa, unidentified; identical to SEQ ID NO: 179
  source 1..1003: note = Description of Unknown: Cas13 sequence

SEQ ID NO: 210 - protein, 1003 aa, unidentified; identical to SEQ ID NO: 198
  source 1..1003: note = Description of Unknown: Cas13 sequence

SEQ ID NO: 211 - protein, 1003 aa, unidentified; identical to SEQ ID NO: 182
  source 1..1003: note = Description of Unknown: Cas13 sequence

SEQ ID NO: 212 - protein, 985 aa, unidentified; identical to SEQ ID NO: 183
  source 1..985: note = Description of Unknown: Cas13 sequence

SEQ ID NO: 213 - protein, 1003 aa, unidentified
  source 1..1003: note = Description of Unknown: Cas13 sequence
MGAIENKHIFAAYANLAIDGLIKTLNFIAKKLDTQKQLSSWDIKHVITLIDSIFDQNPQNNLEQVVEGYLPWIKPIIEMKTPKKGERQSDKLCIEYKTIITAFASLLNDVRNYYTHYYHDPICIYPGGYDIPSSLNCIYDSAINIIKERFQAEEKEMEHLRRYTRKKGRVVLKTEDDHFYYTLANNNDLSEKGYAFFISMFLERKYSYLFLKKLSGFKRGDSLQYRLTLEVFTALSTKPPVERLRTTKDTKQDRALDILNELSRIPIELYQTLEPKYREMYNATLQPTDAEDPYGLPDRSRIRFRSRFEAFALHFLDKQADFKEIGFYTYLGNYFHNGYQKTRVDRETKDRYINFQLAGFCKNIQDISAKKLSEALNVKSIDISTDSIPDINSFEPYLVQSTPHYIVNGNNIGIKVLPEGKDTYPTIDEKGAKMPIADFWLSKYELPAMLFYTYLRNNNIHKSHCPLSVKDIIERSIHKSTKQKHPEERSELMLRRVMKAIFWTDSKLNEVERIKSQKSAFGKRQHEILKAGRIAETLVRDMLWLQPSKNNGRDKVTEPNFQAIQVSLAYFGIRRNDLTEIFTRAGLINSSNPHPFLAQIGTNYTSLIEFYIAYLKERKVYFSRIQKKILQGKLNIQCHPLRDLQREPNKPQEKEEAIFLPRGLFNEAIINCLKKSKLKQLIESPTREKSPALNVSYLILNYFRTYFEDQSQEFYAQPRNYRLFDKLSPNKGKSKSYLSLEQRIKKMEELRPSKIPVAEANKLLEKEDRLYRKNYNEICDNESIIRLYQIQDILLFMMTKEYLPSDLYNRINKYKLENVKGILNERVSYLIDLNPLKIQGEDIKIKDYGKLFYIHHDTRINSLNKVLSKVKRNNSISSSVKIQPYENYKRECLDFEEAQIQIIPIIHSFEIAMVSMFPDLKKATPGNYYDFNELITEYEKRTKQKIDSSFLIKTRNMFLHDKYEAECIKEISDDFVYAKKIIAEFKMKIENIKLEDFSNDSSA

SEQ ID NO: 214 - protein, 1003 aa, unidentified; identical to SEQ ID NO: 195
  source 1..1003: note = Description of Unknown: Cas13 sequence

SEQ ID NO: 215 - protein, 1003 aa, unidentified; identical to SEQ ID NO: 179
  source 1..1003: note = Description of Unknown: Cas13 sequence

SEQ ID NO: 216 - protein, 1359 aa, unidentified
  source 1..1359: note = Description of Unknown: Cas13 sequence
MGKLYGYKRWYEIEQNNGEKGSFKRKIRVKRVYDEESKSYVVRRDEVEDTELIKNKNFIINLKDYKTRNSNIQKFYDKFHVGNILFKLKASVKKGKKTFYKDLEKAETVLTNVEILEQTIKLANNSKINRKKLEKELTQLNINEKSEEVTIDNIKIYLRQGEKFDKKENINAIKWRKLTSEELTIKVEIYRECQSINSNLYSLLDYVLSNEEYDDRYYLEEVENKLLIFGTGNKNKNGRNYYYFDYVLKSLSKIKGLIKKDDRNLNYLMFLFNIQKTSENKEHFINKIFNYFKYEVRIEKEDIVEFLVGELEYYDLIKRIEKKPSENQNQTNLEKTYILLDKHEKLKETIDTKNEIVQKLTIELKNNNLRNRIEIILHKYKILELVDKLNKNIKNGKINTELYGIYKEHYGQCIEYINFNELALEEKELYKIIYRYLKGRIEKLLQNRNKIKIGELRIEDIFIFQKLLEKIEIRVKQYLLEHILYLGKLKHHNIDEVNTIRFVEEHANEELSLELITLFSATNVELNRLLKVKGENGSYEKDYDFFSAKPDKGKIKIKDTNSVMKFELLKKLKFINSEATETNQEAIDFLKEAYNLRNNILHGKNEEIIEDNKKNLSKSYKNINELIEELRPSDNEICKSLNLDIIFKGNRKINDINAKLFGNNREKIYLPSFSKLVPEIKNIIESYDKNGTFNDERIKKIVLNGAIYVNKILYLKECSNKDGEFIKNLKEELNKDSKEKSKYVSIEELYKKSQISASKGNKKAIYKYQRKIIEIYLKYLKENYVEILDFSKLNLNIEQIENDIKNRKNSENKVLIESIKQKVFPENDFEYIISIFALLNDNIFINKIRNRFFSTDTWLKNNRYSNIIKILDEVISVNLLRTELLNTSIDIEEIKDDVLTEDIENIIPEIREEILQRTKKDFKTLLGNNAISKEGLSNEDIDKIRNIEDAKINLDFVNNEIKVSLKEYGSLPPNGVLSRNTSKYYNNEIAKKIDQISILTFTKKSIGTISDEKFRNIYWQERKESDESKKIFVYNKNILYLVTKHSFEKLYKNFLEEELNNLKLEDTKYLRDLDLRREKNLKVDNTLKEINEKVRGYSKEYKKKFIENLKNNDEYFGKVVSGRFKNYQEFKEIYDEVSEYKKIRDVVNFNPLNKVYNYLIEINWKLAIQMARAERDLHYIVNGLNELKLIELNQGQNDGISRAYPKYKLNKEKNKKELRLEECHYNFDIDNYKKFEKICEKLGIDLSENGELQQENETNIRNYISHFYILRKPFVDISISEAIKRVSKLLSYRTRYNNSTYSSVFEVFKKDVELNYDFLKKKIELNGKTYDEVVQRKKISCLELESYLDYKPIIKKILF

SEQ ID NO: 217 - protein, 1003 aa, unidentified; identical to SEQ ID NO: 182
  source 1..1003: note = Description of Unknown: Cas13 sequence

SEQ ID NO: 218 - protein, 1003 aa, unidentified; identical to SEQ ID NO: 179
  source 1..1003: note = Description of Unknown: Cas13 sequence

SEQ ID NO: 219 - protein, 1003 aa, unidentified; identical to SEQ ID NO: 179
  source 1..1003: note = Description of Unknown: Cas13 sequence

SEQ ID NO: 220 - protein, 1359 aa, unidentified; identical to SEQ ID NO: 216
  source 1..1359: note = Description of Unknown: Cas13 sequence

SEQ ID NO: 221 - protein, 1003 aa, unidentified
  source 1..1003: note = Description of Unknown: Cas13 sequence
MGAIENKHIFAAYANLAIDGLIKTLNFIAKKLDTQKQLSSWDIKHVITLIDSIFDQNPQNNLEQVVEGYLPWIKPIIEMKTPKKGERQSDKLCIEYKTIITAFASLLNDVRNYYTHYYHDPICIYPGGYDIPSSLNCIYDSAINIIKERFQAEEKEIEHLRRYTRKKGRVVLKTEDDHFYYTLVNNNDLSEKGYAFFISMFLERKYSYLFLKKLSGFKRGDSLQYRLTLEVFTALSTKPPVERLRTTKDTKQDRALDILNELSRIPIELYQTLEPKYREMYNETLQPTDAEDPYGLPDRSRIRFRSRFEAFALHFLDKQADFKEIGFYTYLGNYFHNGYQKTRVDRETKDRYINFQLAGFCKNIQDISAKKLSEALNVKSIDISTDSIPDINSFEPYLVQSTPHYIVNGNNIGIKVLPEGKDTYPTIDEKGAKMPIADFWLSKYELPAMLFYTYLRNNNIHKSHCPLSVKDIIERSIHKSTKQKHPEERSELMLRRVMKAIFWTDSKLNEVERIKSQKSAFGKRQHEILKAGRIAETLVRDMLWLQPSKNNGRDKVTEPNFQAIQVSLAYFGIRRNDLTEIFTRAGLINSSNPHPFLAQIGTNYTSLIEFYIAYLKERKVYFSRIQKKILQGKLNIQCHPLRDLQREPNKPQDKEEAIFLPRGLFNEAIINCLKKSKLKQLIESPTREKSPALNVSYLIQNYFRTYFEDQSQEFYAQPRNYRLFDKLSPNKGKSKSYLSLEQRIKKMEELRPSKIPVAEANKLLEKEDRLYRKNYNEICDNESIIRLYQIQDILLFMMTKEYLPSDLYNRINKYKLENVKGILNERVSYLIDLNPLKIQGEDIKIKDYGKLFYIHHDTRISSLNKVLSKVKRNNSISSSVKIQPYENYKRECLDFEEAQIQIIPIIHSFEIAMVSMFPDLKKATPGNYYDFNELITEYEKRTKQKIDSSFRIKTRNMFLHDKYEAECIKEISDDFVYAKKIIAEFKMKIENIKLEDLSNDSSA

SEQ ID NO: 222 - protein, 1003 aa, unidentified; identical to SEQ ID NO: 182
  source 1..1003: note = Description of Unknown: Cas13 sequence

SEQ ID NO: 223 - protein, 1003 aa, unidentified
  source 1..1003: note = Description of Unknown: Cas13 sequence
MGAIENKHIFAAYANLAIDGLIKTLNFIAKKLDTQKQLSSWDIKHVITLIDSIFDQNPQNNLEQVVEGYLPWIKPIIEMKTPKKGERQSDKLCIEYKTIITAFASLLNDVRNYYTHYYHDPICIYPGGYDIPSSLNCIYDSAINIIKERFQAEEKEMEHLRRYIRKKGRVVLKTEDDHFYYTLANNNDLSEKGYAFFISMFLERKYSYLFLKKLSGFKRGDSLQYRLTLEVFTALSTKPPVERLRTTKDTKQDRALDILNELSRIPIELYQTLEPKYREMYNETLQPTDAEDPYGLPDRSRIRFRSRFEAFALHFLDKQADFKEIGFYTYLGNYFHNGYQKTRVDRETKDRYINFQLAGFCKNIQDISAKKLSEALNVKSIDISTDSIPDINSFEPYLVQSTPHYIVNGNNIGIKVLPEGKDTYPTIDEKGAKMPIADFWLSKYELPAMLFYTYLRNNNIHKSHCPLSVKDIIERSIHKSTKQKHPEERSELMLRRVMKAIFWTDSKLNEVERIKSQKSAFGKRQHEILKAGRIAETLVRDMLWLQPSKNNGRDKVTEPNFQAIQVSLAYFGIRRNDLTEIFTRAGLINSSNPHPFLAQIGTNYTSLIEFYIAYLKERKVYFSRIQKKILQGKLNIQCHPLRDLQREPNKPQEKEEAIFLPRGLFNEAIINCLKKSKLKQLIESPTREKSPVLNVSYLIQNYFRAYFEDQSQEFYAQPRNYRLFDKLSPNKGKSKSYLSLEQRIKKMEELRPSKIPVAEANKLLEKEDRLYRKNYNEICDNESIIRLYQIQDILLFMMTKEYLPSDLYNRINKYKLENVKGILNERVSYLIDLNPLKIQGEDIKIKDYGKLFYIHHDTRISSLNKVLSKVKRNNSISSSVKIQPYENYKRECLDFEEAQIQIIPIIHSFEIAMVSMFPDLKKATPGNYYDFNELITEYEKRTKQKIDSSFLIKTRNMFLHDKYEAECIKEISDDFVYAKKIIAEFKMKIENIKLEDFSNDSSA

SEQ ID NO: 224 - protein, 985 aa, unidentified; identical to SEQ ID NO: 208
  source 1..985: note = Description of Unknown: Cas13 sequence

SEQ ID NO: 225 - protein, 1003 aa, unidentified; identical to SEQ ID NO: 179
  source 1..1003: note = Description of Unknown: Cas13 sequence

SEQ ID NO: 226 - protein, 1003 aa, unidentified; identical to SEQ ID NO: 198
  source 1..1003: note = Description of Unknown: Cas13 sequence

SEQ ID NO: 227 - protein, 1003 aa, unidentified; identical to SEQ ID NO: 182
  source 1..1003: note = Description of Unknown: Cas13 sequence

SEQ ID NO: 228 - protein, 1003 aa, unidentified; identical to SEQ ID NO: 182
  source 1..1003: note = Description of Unknown: Cas13 sequence

SEQ ID NO: 229 - protein, 1095 aa, unidentified
  source 1..1095: note = Description of Unknown: Cas13 sequence
MEKPLLPNVYTLKHKFFWGAFLNIARHNAFITICHINEQLGLKTPSNDDKIVDVVCETWNNILNNDHDLLKKSQLTELILKHFPFLTAMCYHPPKKEGKKKGHQKEQQKEKESEAQSQAEALNPSKLIEALEILVNQLHSLRNYYSHYKHKKPDAEKDIFKHLYKAFDASLRMVKEDYKAHFTVNLTRDFAHLNRKGKNKQDNPDFNRYRFEKDGFFTESGLLFFTNLFLDKRDAYWMLKKVSGFKASHKQREKMTTEVFCRSRILLPKLRLESRYDHNQMLLDMLSELSRCPKLLYEKLSEENKKHFQVEADGFLDEIEEEQNPFKDTLIRHQDRFPYFALRYLDLNESFKSIRFQVDLGTYHYCIYDKKIGDEQEKRHLTRTLLSFGRLQDFTEINRPQEWKALTKDLDYKETSNQPFISKTTPHYHITDNKIGFRLGTSKELYPSLEIKDGANRIAKYPYNSGFVAHAFISVHELLPLMFYQHLTGKSEDLLKETVRHIQRIYKDFEEERINTIEDLEKANQGRLPLGAFPKQMLGLLQNKQPDLSEKAKIKIEKLIAETKLLSHRLNTKLKSSPKLGKRREKLIKTGVLADWLVKDFMRFQPVAYDAQNQPIKSSKANSTEFWFIRRALALYGGEKNRLEGYFKQTNLIGNTNPHPFLNKFNWKACRNLVDFYQQYLEQREKFLEAIKNQPWEPYQYCLLLKIPKENRKNLVKGWEQGGISLPRGLFTEAIRETLSEDLMLSKPIRKEIKKHGRVGFISRAITLYFKEKYQDKHQSFYNLSYKLEAKAPLLKREEHYEYWQQNKPQSPTESQRLELHTSDRWKDYLLYKRWQHLEKKLRLYRNQDVMLWLMTLELTKNHFKELNLNYHQLKLENLAVNVQEADAKLNPLNQTLPMVLPVKVYPATAFGEVQYHKTPIRTVYIREEHTKALKMGNFKALVKDRRLNGLFSFIKEENDTQKHPISQLRLRRELEIYQSLRVDAFKETLSLEEKLLNKHTSLSSLENEFRALLEEWKKEYAASSMVTDEHIAFIASVRNAFCHNQYPFYKEALHAPIPLFTVAQPTTEEKDGLGIAEALLKVLREYCEIVKSQI

SEQ ID NO: 230 - protein, 1095 aa, unidentified; identical to SEQ ID NO: 229
  source 1..1095: note = Description of Unknown: Cas13 sequence

SEQ ID NO: 231 - protein, 1008 aa, unidentified; identical to SEQ ID NO: 60
  source 1..1008: note = Description of Unknown: Cas13 sequence

SEQ ID NO: 232 - protein, 1095 aa, unidentified; identical to SEQ ID NO: 229
  source 1..1095: note = Description of Unknown: Cas13 sequence

SEQ ID NO: 233 - protein, 1224 aa, unidentified
  source 1..1224: note = Description of Unknown: Cas13 sequence
MENKTSLGNNIYYNPFKPQDKSYFAGYFNAAMENTDSVFRELGKRLKGKEYTSENFFDAIFKENISLVEYERYVKLLSDYFPMARLLDKKEVPIKERKENFKKNFKGIIKAVRDLRNFYTHKEHGEVEITDEIFGVLDEMLKSTVLTVKKKKVKTDKTKEILKKSIEKQLDILCQKKLEYLRDTARKIEEKRRNQRERGEKELVAPFKYSDKRDDLIAAIYNDAFDVYIDKKKDSLKESSKAKYNTKSDPQQEEGDLKIPISKNGVVFLLSLFLTKQEIHAFKSKIAGFKATVIDEATVSEATVSHGKNSICFMATHEIFSHLAYKKLKRKVRTAEINYGEAENAEQLSVYAKETLMMQMLDELSKVPDVVYQNLSEDVQKTFIEDWNEYLKENNGDVGTMEEEQVIHPVIRKRYEDKFNYFAIRFLDEFAQFPTLRFQVHLGNYLHDSRPKENLISDRRIKEKITVFGRLSELEHKKALFIKNTETNEDREHYWEIFPNPNYDFPKENISVNDKDFPIAGSILDREKQPVAGKIGIKVKLLNQQYVSEVDKAVKAHQLKQRKASKPSIQNIIEEIVPINESNPKEAIVFGGQPTAYLSMNDIHSILYEFFDKWEKKKEKLEKKGEKELRKEIGKELEKKIVGKIQAQIQQIIDKDTNAKILKPYQDGNSTAIDKEKLIKDLKQEQNILQKLKDEQTVREKEYNDFIAYQDKNREINKVRDRNHKQYLKDNLKRKYPEAPARKEVLYYREKGKVAVWLANDIKRFMPTDFKNEWKGEQHSLLQKSLAYYEQCKEELKNLLPEKVFQHLPFKLGGYFQQKYLYQFYTCYLDKRLEYISGLVQQAENFKSENKVFKKVENECFKFLKKQNYTHKELDARVQSILGYPIFLERGFMDEKPTIIKGKTFKGNEALFADWFRYYKEYQNFQTFYDTENYPLVELEKKQADRKRKTKIYQQKKNDVFTLLMAKHIFKSVFKQDSIDQFSLEDLYQSREERLGNQERARQTGERNTNYIWNKTVDLKLCDGKITVENVKLKNVGDFIKYEYDQRVQAFLKYEENIEWQAFLIKESKEEENYPYVVEREIEQYEKVRREELLKEVHLIEEYILEKVKDKEILKKGDNQNFKYYILNGLLKQLKNEDVESYKVFNLNTEPEDVNINQLKQEATDLEQKAFVLTYIRNKFAHNQLPKKEFWDYCQEKYGKIEKEKTYAEYFAEVFKKEKEALIK

SEQ ID NO: 234 - protein, 1095 aa, unidentified; identical to SEQ ID NO: 229
  source 1..1095: note = Description of Unknown: Cas13 sequence

SEQ ID NO: 235 - protein, 1174 aa, unidentified
  source 1..1174: note = Description of Unknown: Cas13 sequence
MTEQSERPYNGTYYTLEDKHFWAAFLNLARHNAYITLTHIDRQLAYSKADITNDQDVLSFKALWKNLDNDLERKSRLRSLILKHFSFLEGAAYGKKLFESKSSGNKSSKNKELTKKEKEELQANALSLDNLKSILFDFLQKLKDFRNYYSHYRHSGSSELPLFDGNMLQRLYNVFDVSVQRVKRDHEHNDKVDPHYHFNHLVRKGKKDRYGHNDNPSFKHHFVDSEGMVTEAGLLFFVSLFLEKRDAIWMQKKIRGFKGGTGPYEQMTNEVFCRSRISLPKLKLESLRTDDWMLLDMLNELVRCPKPLYDRLREKDRACFRVPVDILPDEDDTDGGGEDPFKNTLVRHQDRFPYFALRYFDLKKVFTSLRFHIDLGTYHFAIYKKMIGEQPEDRHLTRNLYGFGRIQDFAEEHRPEEWKRLVRDLDYLETGDKPYISQTTPHYHIEKGKIGLRFVPEGQHLWPSPEVGTTRTGRSKCAQDKRLTAEAFLSVHELMPMMFYYFLLREKYSEEVSAEKVQGRIKRVIEDVYAIYDAFARDEINTLKELDTCLADKGIRRGHLPKQMITILSQERKDMKEKIRKKLQEMIADTDHRLDMLDRQTDRKIRIGRKNAGLPKSGVIADWLVRDMMRFQPVAKDASGKPLNNSKANSTEYRMLQRALALFGGEKERLTPYFRQMNLTGGNNPHPFLHETRWESHTNILSFYRSYLRARKAFLERIGRSDRVENCPFLLLKEPKTDRQTLVAGWKDEFHLPRGIFTEAVRDCLIEMGYDEVGSYREVGFMAKAVPLYFERACEDRVQPFYDSPFNVGNSLKPKKGRFLSKEDRAEEWERGMERFRDLEAWSHSAARRIKDAFAGIEYASPGNKKKIEQLLRDLSLWEAFESKLKVRADKINLAKLKKEILEAQEHPYHDFKSWQKFERELRLVKNQDIITWMMCRDLMEENKVEGLDTGTLYLKDIRPNVQEQGSLNVLNRVKPMRLPVVVYRADSRGHVHKEAPLATVYIEERNTKLLKQGNFKSFVKDRRLNGLFSFVDTGGLAMEQYPISKLRVEYELAKYQTARVCVFELTLRLEESLLSRYPHLPDESFREMLESWSDPLLAKWPELHGKVRLLIAVRNAFSHNQYPMYDEAVFSSIRKYDPSSPDAIEERMGLNIAHRLSEEVKQAKETVERIIQA

SEQ ID NO: 236 - protein, 1103 aa, unidentified
  source 1..1103: note = Description of Unknown: Cas13 sequence
MEKPLPPNVYTLKHKFFWGAFLNIARHNAFITICHINEQLGLTTPPNDDKIADVVCGTWNNILNNDHDLLKKSQLTELILKHFPFLAAMCYHPPKKEGKKKGSQKEQQKEKENEAQSQAEALNPSELIKALKTLVKQLRTLRNYYSHYKHKKPDAEKDIFKHLYKAFDASLRMVKEDYKAHFTVNLTQDFAHLNRKGKNKQDNPKFDRYRFEKDGFFTESGLLFFTNLFLDKRDAYWMLKKVSGFKASHKQSEKMTTEVFCRSRILLPKLRLESRYDHNQMLLDMLSELSRCPKLLYEKLSEKDKKHFQVEADGFLDEIEEEQNPFKDALIRHQDRFPYFALRYLDLNESFKSIRFQVDLGTYHYCIYDKKIGDEQEKRHLTRTLLSFGRLQDFTEINRPQEWKALTKDLDYKETSKQPFISKTTPHYHITDNKIGFRLGTSKELYPSLEVKDGANRIAKYPYNSDFVAHAFISVHELLPLMFYQHLTGKSEDLLKETVRHIQRIYKDFEEERINTIEDLEKANQGRLPLGAFPKQMLGLLQNKQPDLSEKAKIKIEKLIAETKLLSHRLNTKLKSSPKLGKRREKLIKTGDWLVKDFMRFQPVAYDVQNQPIESSKANSTEFQLIQRALALYGGEKNRLEGYFKQTNLIGNTNPHPFLNKFNWKACRNLVDFYQQYLEQREKFLEAIKNQPWEPYQYCLLLKIPKENRKNLVKGWEQGGISLPRGLFTEAIRETLSEDLTLSKPIRKEIKKHGRVGFISRAITLYFRERYQDDHQSFYNLPYELEAKASTPKPPLPKKREYVLRAEHYEYWQQNKPQSPTELQRLELHTSDRWKDYLLYKRWQHLEKKLRLYRNQDVMLWLMTLELTKNHFKELKLNYHQLKLENLAVNVQEADAKLNPLNQTLPMVLPVKVYPATAFGEVQYQETPIRTVYIREEQTKALKMGNFKALVKDRRLNGLFSFIKEENDTQKHPISQLRLRRELEIYQSLRVDAFKETLSLEEKLLNKHASLSSLENEFRTLLEEWKKKYAASSMVTDEHIAFIASVRNAFCHNQYPFYKETLHAPILLFTVAQPTTEEKDGLGIAEALLRVLREYCEIVKSQI

SEQ ID NO: 237 - protein, 1095 aa, unidentified; identical to SEQ ID NO: 229
  source 1..1095: note = Description of Unknown: Cas13 sequence

SEQ ID NO: 238 - protein, 1224 aa, unidentified; identical to SEQ ID NO: 233
  source 1..1224: note = Description of Unknown: Cas13 sequence

SEQ ID NO: 239 - protein, 948 aa, unidentified
  source 1..948: note = Description of Unknown: Cas13 sequence
MFFSFHNAQRVIFKHLYKAFDASLRMVKEDYKAHFTVNLTRDFAHLNRKGKNKQDNPDFNRYRFEKDGFFTESGLLFFTNLFLDKRDAYWMLKKVSGFKASHKQREKMTTEVFCRSRILLPKLRLESRYDHNQMLLDMLSELSRCPKLLYEKLSEENKKHFQVEADGFLDEIEEEQNPFKDTLIRHQDRFPYFALRYLDLNESFKSIRFQVDLGTYHYCIYDKKIGDEQEKRHLTRTLLSFGRLQDFTEINRPQEWKALTKDLDYKETSNQPFISKTTPHYHITDNKIGFRLGTSKELYPSLEIKDGANRIAKYPYNSGFVAHAFISVHELLPLMFYQHLTGKSEDLLKETVRHIQRIYKDFEEERINTIEDLEKANQGRLPLGAFPKQMLGLLQNKQPDLSEKAKIKIEKLIAETKLLSHRLNTKLKSSPKLGKRREKLIKTGVLADWLVKDFMRFQPVAYDAQNQPIKSSKANSTEFWFIRRALALYGGEKNRLEGYFKQTNLIGNTNPHPFLNKFNWKACRNLVDFYQQYLEQREKFLEAIKNQPWEPYQYCLLLKIPKENRKNLVKGWEQGGISLPRGLFTEAIRETLSEDLMLSKPIRKEIKKHGRVGFISRAITLYFKEKYQDKHQSFYNLSYKLEAKAPLLKREEHYEYWQQNKPQSPTESQRLELHTSDRWKDYLLYKRWQHLEKKLRLYRNQDVMLWLMTLELTKNHFKELNLNYHQLKLENLAVNVQEADAKLNPLNQTLPMVLPVKVYPATAFGEVQYHKTPIRTVYIREEHTKALKMGNFKALVKDRRLNGLFSFIKEENDTQKHPISQLRLRRELEIYQSLRVDAFKETLSLEEKLLNKHTSLSSLENEFRALLEEWKKEYAASSMVTDEHIAFIASVRNAFCHNQYPFYKEALHAPIPLFTVAQPTTEEKDGLGIAEALLKVLREYCEIVKSQI

SEQ ID NO: 240 - protein, 1095 aa, unidentified; identical to SEQ ID NO: 229
  source 1..1095: note = Description of Unknown: Cas13 sequence

SEQ ID NO: 241 - protein, 1106 aa, unidentified
  source 1..1106: note = Description of Unknown: Cas13 sequence
MEKPLPPNVYTLKHKFFWGAFLNIARHNAFITICHINEQLGLTTPPNDDKIADVVCGTWNNILNNDHDLLKKSQLTELILKHFPFLAAMCYHPPKKEGKKKGSQKEQQKEKENEAQSQAEALNPSELIKALKTLVKQLRTLRNYYSHYKHKKPDAEKDIFKHLYKAFDASLRMVKEDYKAHFTVNLTQDFAHLNRKGKNKQDNPKFDRYRFEKDGFFTESGLLFFTNLFLDKRDAYWMLKKVSGFKASHKQSEKMTTEVFCRSRILLPKLRLESRYDHNQMLLDMLSELSRCPKLLYEKLSEKDKKHFQVEADGFLDEIEEEQNPFKDALIRHQDRFPYFALRYLDLNESFKSIRFQVDLGTYHYCIYDKKIGDEQEKRHLTRTLLSFGRLQDFTEINRPQEWKALTKDLDYKETSKQPFISKTTPHYHITDNKIGFRLGTSKELYPSLEVKDGANRIAKYPYNSDFVAHAFISVHELLPLMFYQHLTGKSEDLLKETVRHIQRIYKDFEEERINTIEDLEKANQGRLPLGAFPKQMLGLLQNKQPDLSEKAKIKIEKLIAETKLLSHRLNTKLKSSPKLGKRREKLIKTGVLADWLVKDFMRFQPVAYDVQNQPIESSKANSTEFQLIQRALALYGGEKNRLEGYFKQTNLIGNTNPHPFLNKFNWKACRNLVDFYQQYLEQREKFLEAIKNQPWEPYQYCLLLKIPKENRKNLVKGWEQGGISLPRGLFTEAIRETLSEDLTLSKPIRKEIKKHGRVGFISRAITLYFRERYQDDHQSFYNLPYELEAKASTPKPPLPKKREYVLRAEHYEYWQQNKPQSPTELQRLELHTSDRWKDYLLYKRWQHLEKKLRLYRNQDVMLWLMTLELTKNHFKELKLNYHQLKLENLAVNVQEADAKLNPLNQTLPMVLPVKVYPATAFGEVQYQETPIRTVYIREEQTKALKMGNFKALVKDRRLNGLFSFIKEENDTQKHPISQLRLRRELEIYQSLRVDAFKETLSLEEKLLNKHASLSSLENEFRTLLEEWKKKYAASSMVTDEHIAFIASVRNAFCHNQYPFYKETLHAPILLFTVAQPTTEEKDGLGIAEALLRVLREYCEIVKSQI

SEQ ID NO: 242 - protein, 1095 aa, unidentified; identical to SEQ ID NO: 229
  source 1..1095: note = Description of Unknown: Cas13 sequence

SEQ ID NO: 243 - protein, 1095 aa, unidentified; identical to SEQ ID NO: 229
  source 1..1095: note = Description of Unknown: Cas13 sequence

SEQ ID NO: 244 - protein, 1095 aa, unidentified; identical to SEQ ID NO: 229
  source 1..1095: note = Description of Unknown: Cas13 sequence

SEQ ID NO: 245 - protein, 948 aa, unidentified; identical to SEQ ID NO: 239
  source 1..948: note = Description of Unknown: Cas13 sequence

SEQ ID NO: 246 - protein, 1008 aa, unidentified; identical to SEQ ID NO: 60
  source 1..1008: note = Description of Unknown: Cas13 sequence

SEQ ID NO: 247 - protein, 1175 aa, unidentified
  source 1..1175: note = Description of Unknown: Cas13 sequence
MTEQNEKPYNGTYYTLEDKHFWAAFLNLARHNAYITLTHIDRQLAYSKADITNDEDILFFKGQWKNLDNDLERKARLRSLILKHFFFLEGAAYGKKLFESQSSGNKSSKKKELTKKEKEELQANALSLDNLKSILFDFLQKLKDFRNYYSHYRHPESSELPLFDGNMLQRLYNVFDVSVQRVKRDHEHNDKVDPHRHFNHLVRKGKKDKYGNNDNPFFKHHFVDREGKVTEAGLLFFVSLFLEKRDAIWMQKKIRGFKGGTETYQQMTNEVFCRSRISLPKLKLESLRTDDWMLLDMLNELVRCPKSLYDRLREEDRARFRVPVDILSDEDDTDGAEEDPFKNTLVRHQDRFPYFALRYFDLKKVFTSLRFHIDLGTYHFAIYKKNIGEQPEDRHLTRNLYGFGRIQDFAEEHRPEEWKRLVRDLDYFETGDKPYITQTTPHYHIEKGKIGLRFVPEGQHLWPSPEVGATRTGRSKYAQDKRFTAEAFLSVHELMPMMFYYFLLREKYSEEASAERVQGRIKRVIEDVYAVYDAFARDEINTRDELDACLADKGIRRGHLPRQMIAILSQKHKDMEEKVRKKLQEMIADTDHRLDMLDRQTDRKIRIGRKNAGLPKSGVIADWLVRDMMRFQPVAKDTSGKPLNNSKANSTEYRMLQRALALFGGEKERLTPYFRQMNLTGGNNPHPFLHETRWESHTNILSFYRSYLKARKAFLQSIGRSDRVENHRFLLLKEPKTDRQTLVAGWKGEFHLPRGIFTEAVRDCLIEMGLDEVRSYKEVGFMAKAVPLYFERASKDRVQPFYDYPFNVGNSLKPKKGRFLSKEKRAEEWESGKERFRDLEAWSHSAARRIEDAFAGIENASRENKKKIEQLLQDLSLWETFESKLKVKADKINIAKLKKEILEAKEHPYLDFKSWQKFERELRLVKNQDIITWMMCRDLMEENKVEGLDTGTLYLKDIRTDVQEQGSLNVLNHVKPMRLPVVVYRADSRGHVHKEQAPLATVYIEERDTKLLKQGNFKSFVKDRRLNGLFSFVDTGGLAMEQYPISKLRVEYELAKYQTARVCAFEQTLELEESLLTRYPHLPDKNFRKMLESWSDPLLAKWPELHEKVRLLIAVRNAFSHNQYPMYDEAVFSPIRKYDPSSPDAIEERMRLNIAHRLSEEVKQAKETVERIIQA

SEQ ID NO: 248 - protein, 1008 aa, unidentified; identical to SEQ ID NO: 60
  source 1..1008: note = Description of Unknown: Cas13 sequence

SEQ ID NO: 249 - protein, 1003 aa, unidentified; identical to SEQ ID NO: 213
  source 1..1003: note = Description of Unknown: Cas13 sequence

SEQ ID NO: 250 - protein, 1106 aa, unidentified; identical to SEQ ID NO: 241
  source 1..1106: note = Description of Unknown: Cas13 sequence

SEQ ID NO: 251 - protein, 1095 aa, unidentified; identical to SEQ ID NO: 229
  source 1..1095: note = Description of Unknown: Cas13 sequence

SEQ ID NO: 252 - protein, 1095 aa, unidentified; identical to SEQ ID NO: 229
  source 1..1095: note = Description of Unknown: Cas13 sequence

SEQ ID NO: 253 - protein, 948 aa, unidentified; identical to SEQ ID NO: 239
  source 1..948: note = Description of Unknown: Cas13 sequence

SEQ ID NO: 254 - protein, 1095 aa, unidentified; identical to SEQ ID NO: 229
  source 1..1095: note = Description of Unknown: Cas13 sequence

SEQ ID NO: 255 - protein, 1095 aa, unidentified; identical to SEQ ID NO: 229
  source 1..1095: note = Description of Unknown: Cas13 sequence

SEQ ID NO: 256 - protein, 1095 aa, unidentified; identical to SEQ ID NO: 229
  source 1..1095: note = Description of Unknown: Cas13 sequence

SEQ ID NO: 257 - protein, 1008 aa, unidentified; identical to SEQ ID NO: 60
  source 1..1008: note = Description of Unknown: Cas13 sequence

SEQ ID NO: 258 - protein, 948 aa, unidentified; identical to SEQ ID NO: 239
  source 1..948: note = Description of Unknown: Cas13 sequence

SEQ ID NO: 259 - protein, 1095 aa, unidentified; identical to SEQ ID NO: 229
  source 1..1095: note = Description of Unknown: Cas13 sequence

SEQ ID NO: 260 - protein, 1131 aa, unidentified
  source 1..1131: note = Description of Unknown: Cas13 sequence
MTNTPKRRTLHRHPSYFGAFLNIARHNAFMIMEHLSTKYDMEDKNTLDEAQLPNAKLFGCLKKRYGKPDVTEGVSRDLRRYFPFLNYPLFLHLEKQQNAEQAATYDINPEDIEFTLKGFFRLLNQMRNNYSHYISNTDYGKFDKLPVQDIYEAAIFRLLDRGKHTKRFDVFESKHTRHLESNNSEYRPRSLANSPDHENTVAFVTCLFLERKYAFPFLSRLDCFRSTNDAAEGDPLIRKASHECYTMFCCRLPQPKLESSDILLDMVNELGRCPSALYNLLSEEDQARFHIKREEITGFEEDPDEELEQEIVLKRHSDRFPYFALRYFDDTEAFQTLRFDVYLGRWRTKPVYKKRIYGQERDRVLTQSIRTFTRLSRLLPIYENVKHDAVRQNEEDGKLVNPDVTSQFHKSWIQIESDDRAFLSDRIEHFSPHYNFGDQVIGLKFINPDRYAAIQNVFPKLPGEEKKDKDAKLVNETADAIISTHEIRSLFLYHYLSKKPISAGDERRFIQVDTETFIKQYIDTIKLFFEDIKSGELQPIADPPNYQKNEPLPYVRGDKEKTQEERAQYRERQKEIKERRKELNTLLQNRYGLSIQYIPSRLREYLLGYKKVPYEKLALQKLRAQRKEVKKRIKDIEKMRTPRVGEQATWLAEDIVFLTPPKMHTPERKTTKHPQKLNNDQFRIMQSSLAYFSVNKKAIKKFFQKETGIGLSNRETSHPFLYRIDVGRCRGILDFYTGYLKYKMDWLDDAIKKVDNRKHGKKEAKKYEKYLPSSIQHKTPLELDYTRLPVYLPRGLFKKAIVKALAAHADFQVEPEEDNVIFCLDQLLDGDTQDFYNWQRYYRSALTEKETDNQLVLAHPYAEQILGTIKTLEGKQKNNKLGNKAKQKIKDELIDLKRAKRRLLDREQYLRAVQAEDRALWLMIQERQKQKAEHEEIAFDQLDLKNITKILTESIDARLRIPDTKVDITDKLPLRRYGDLRRVAKDRRLVNLASYYHVAGLSEIPYDLVKKELEEYDRRRVAFFEHVYQFEKEVYDRYAAELRNENPKGESTYFSHWEYVAVAVKHSADTHFNELFKEKVMQLRNKFHHNEFPYFDWLLPEVEKASAALYADRVFDVAEGYYQKMRKLMRQ

SEQ ID NO: 261 - protein, 1131 aa, unidentified; identical to SEQ ID NO: 260
  source 1..1131: note = Description of Unknown: Cas13 sequence

SEQ ID NO: 262 - protein, 1095 aa, unidentified; identical to SEQ ID NO: 229
  source 1..1095: note = Description of Unknown: Cas13 sequence

SEQ ID NO: 263 - protein, 1095 aa, unidentified; identical to SEQ ID NO: 229
  source 1..1095: note = Description of Unknown: Cas13 sequence

SEQ ID NO: 264 - protein, 1095 aa, unidentified; identical to SEQ ID NO: 229
  source 1..1095: note = Description of Unknown: Cas13 sequence

SEQ ID NO: 265 - protein, 1115 aa, unidentified; identical to SEQ ID NO: 58
  source 1..1115: note = Description of Unknown: Cas13 sequence

SEQ ID NO: 266 - protein, 1099 aa, unidentified
  source 1..1099: note = Description of Unknown: Cas13 sequence
MEKPLPPNVYTLKHKFFWGAFLNIARHNAFITICHINEQLGLTTPPNDDKIADVVCGTWNNILNNDHDLLKKSQLTELILKHFPFLAAMCYHPPKKEGKKKGSQKEQQKEKEKEKENEAQSQAEALNPSELIKVLKTIVKQLRTLRNYYSHHSHKKPDTEKDIFKHLYKAFDASLRMVKEDYKAHFTVNLTRDFAHLNRKGKNKQDNPDFNRYRFEKDGFFTESGLLFFTNLFLDKRDAYWMLKKVSGFKASHKQREKMTTEVFCRSHILLPKLRLESRYDHNQMLLDMLSELSRCPKLLYEKLSEENKKHFQVEADGFLDEIEEEQNPFKDTLIRHQDRFPYFALRYLDLNESFKSIRFQVDLGTYHYCIYDKKIGDEQEKRHLTRTLLSFGRLQDFTEINRPQEWKALTKDLDYKETSNQPFISKTTPHYHITDNKIGFRLGTSKELYPSLEIKDGANRIAKYPYNSGFVAHAFISVHELLPLMFYQHLTGKSEDLLKETVRHIQRIYKDFEEERINTIEDLEKANQGRLPLGAFPKQMLGLLQNKQPDLSEKAKIKIEKLIAETKLLSHRLNTKLKSSPKLGKRREKLIKTGVLADWLVKDFMRFQPVAYDAQNQPIKSSKANSTEFWFIRRALALYGGEKNRLEGYFKQTNLIGNTNPHPFLNKFNWKACRNLVDFYQQYLEQREKFLEAIKNQPWEPYQYCLLLKIPKENRKNLVKGWEQGGISLPRGLFTEAIRETLSEDLMLSKPIRKEIKKHGRVGFISRAITLYFKEKYQDKHQSFYNLSYKLEAKAPLLKREEHYEYWQQNKPQSPTESQRLELHTSDRWKDYLLYKRWQHLEKKLRLYRNQDIMLWLMTLELTKNHFKELNLNYHQLKLENLAVNVQEADAKLNPLNQTLPMVLPVKVYPATAFGEVQYQETPIRTVYIREEHTKALKMGNFKALVKDRRLNGLFSFIKEENDTQKHPISQLRLRRELEIYQSLRVDAFKETLDLEEKLLKKHTSLSSLENKFRILLEEWKKEYAASSMVTDEHIAFIASVRNAFCHNQYPFYEEALHAPIPLFTVAQQTTEEKDGLGIAEALLRVLREYCEIVKSQI

SEQ ID NO: 267 - protein, 948 aa, unidentified; identical to SEQ ID NO: 239
  source 1..948: note = Description of Unknown: Cas13 sequence

SEQ ID NO: 268 - protein, 1099 aa, unidentified; identical to SEQ ID NO: 266
  source 1..1099: note = Description of Unknown: Cas13 sequence

SEQ ID NO: 269 - protein, 1003 aa, unidentified; identical to SEQ ID NO: 193
  source 1..1003: note = Description of Unknown: Cas13 sequence

SEQ ID NO: 270 - protein, 1095 aa, unidentified
  source 1..1095: note = Description of Unknown: Cas13 sequence
MEKPLLPNVYTLKHKFFWGAFLNIARHNAFITICHINEQLGLKTPSNDDKIVDVVCETWNNILNNDHDLLKKSQLTELILKHFPFLTAMCYHPPKKEGKKKGHQKEQQKEKESEAQSQAEALNPSKLIEALEILVNQLHSLRNYYSHYKHKKPDAEKDIFKHLYKAFDASLRMVKEDYKAHFTVNLTRDFAHLNRKGKNKQDNPDFNRYRFEKDGFFTESGLLFFTNLFLDKRDAYWMLKKVSGFKASHKQREKMTTEVFCRSRILLPKLRLESRYDHNQMLLDMLSELSRCPKLLYEKLSEENKKHFQVEADGFLDEIEEEQNPFKDTLIRHQDRFPYFALRYLDLNESFKSIRFQVDLGTYHYCIYDKKIGDEQEKRHLTRTLLSFGRLQDFTEINRPQEWKALTKDLDYKETSNQPFISKTTPHYHITDNKIGFRLGTSKELYPSLEIKDGANRIAKYPYNSGFVAHAFISVHELLPLMFYQHLTGKSEDLLKETVRHIQRIYKDFEEERINTIEDLEKANQGRLPLGAFPKQMLGLLQNKQPDLSEKAKIKIEKLIAETKLLSHRLNTKLKSSPKLGKRREKLIKTGVLADWLVKDFMRFQPVAYDAQNQPIKSSKANSTEFWFIRRALALYGGEKNRLEGYFKQTNLIGNTNPHPFLNKFNWKACRNLVDFYQQYLEQREKFLEAIKNQPWEPYQYCLLLKIPKENRKNLVKDWEQGGISLPRGLFTEAIRETLSEDLMLSKPIRKEIKKHGRVGFISRAITLYFKEKYQDKHQSFYNLSYKLEAKAPLLKREEHYEYWQQNKPQSPTESQRLELHTSDRWKDYLLYKRWQHLEKKLRLYRNQDVMLWLMTLELTKNHFKELNLNYHQLKLENLAVNVQEADAKLNPLNQTLPMVLPVKVYPATAFGEVQYHKTPIRTVYIREEHTKALKMGNFKALVKDRRLNGLFSFIKEENDTQKHPISQLRLRRELEIYQSLRVDAFKETLSLEEKLLNKHTSLSSLENEFRALLEEWKKEYAASSMVTDEHIAFIASVRNAFCHNQYPFYKEALHAPIPLFTVAQPTTEEKDGLGIAEALLKVLREYCEIVKSQI

SEQ ID NO: 271 - protein, 1106 aa, unidentified; identical to SEQ ID NO: 241
  source 1..1106: note = Description of Unknown: Cas13 sequence

SEQ ID NO: 272 - protein, 1095 aa, unidentified; identical to SEQ ID NO: 229
  source 1..1095: note = Description of Unknown: Cas13 sequence

SEQ ID NO: 273 - protein, 1095 aa, unidentified; identical to SEQ ID NO: 229
  source 1..1095: note = Description of Unknown: Cas13 sequence

SEQ ID NO: 274 - protein, 1335 aa, unidentified
  source 1..1335: note = Description of Unknown: Cas12a sequence
MKSLAQFQNLYALQKTLRFELKPEGHTRETFNRWLEEIEKEQASENENIVYQDLLRAKKYEKIKIILDEYHKDFIEQALAYANLTELEKYEELYRKSNRTSEEEEEFENTKESLRKQIANIFIKNPNKTVQERWKFLFSKKLIQNELIVWVKGNYELLSEKLKNEFPDESSIISTIEDFKYFTTYFRNYHENRKNLYSNEDKFSTIAHRLIHENLPKFIDNIAIYQKAKAVLNINEVEKELGLPEDTLDKIFSLDFFSKALTQKGIDQYNYFLGGKTENEVKKIKGLNEFINLYNQQQQDKNQRLPFLKVLYKLPLFERTSTSFRFEPIENDRDLIERIGKFYYNDLKQYRDDSQGDTTDILSGINTLLRHVHDYRDGLYVNGGITLTQISQKIFGSWSYINNALAYFYDTYIDASGVDHQGERKPKKQKQIQEKTKWLKQKQFPVILVEKALSEYKSIETNEDLKTRISDTTLCDFFKRCGNDDNGQDLFDRIEARLREKNEEGYSLEDLLKKEFTTERKLMQDKTKTLLIKNFLDVIQGDKDDITAGLLHFVKCLIPRTEISEKNELFYSGMEKYLNILSEVTPLYNKARNYLTQKPYSIEKVKLNFENSTLLDGWDENEESDNSCVLLRKRGYYYLGIMNKKHNMIFDRKIYPKATEGEAYYEKMIYKLLPGAYKMLPKVFFSEKNIDYFKPSEEILRIRNTASYSKNGQPQEGYQKASFSIEDCRKYIDFFKKCIANHWDWQKFNFNFSPTEYYQSIDEFYREIERQGYKIDFVKIPESYINQLIKENKLYLFKIYNKDFSEKKKSKGKDNLHTLYWKMLFDEKNLKDVVLKLNGEAEVFFRQKSILYNEEIWNKGHHYSELKDRFSYPIISNKRYAEDKFFLHVPITLNFKADGINNVNNMVNEFIKDNRDIHIIGIDRGERHLLYVSVINQKGDIVEQCSLNEIVTEYNGKIFKKNYHEELDNLEKERDRARKDWQTIANIKELKEGYLSHVIHKISKLILKYNAIVVMEDLNSGFKRGRQKVEKQVYQNFEKQLIEKLNYLVLKESNVDEPGGVLRAYQLANKFETFKKLGKQSGIIFYVPAAYTSAIDPVTGYIQYLYPLKQADSVEKARKFYSQFKRISYNPHKQWFEFSFDYNDFNIIYHGKSSWTICTTNTERYMWNRLLNNGHGGEELVYVTNELELLFGEYNIIYGDGKDIKQQITDVQDIDVDRTAKQFYKRINELLNLTLKLRHNNGKKGADEEDYILSPVEPYFDSRFESRKPSMQQTLPINADANGAFNIARKGLLLLERLNQLGVEEFEKTKKSNNKKTQWLPHELWVEYAQNHTRK

SEQ ID NO: 275 - protein, 1273 aa, unidentified
  source 1..1273: note = Description of Unknown: Cas12a sequence
MQDKTGWSSFTNKYSLSKTLRFELKPVGNTQKMLEDDGVFQKDRERQENYKKVKPFMDKLHREFIKEALNNLKLEGLTEYFEIFKKFRKDKNNKELKNAEKKLRQIIGRCYTETAQIWVEKYKEFGFKKKNIGFLFEEGVFELMKLKYGNDEASQIEKNGEVLSIFDGWKGFLGYFKKFFETRNNFYKDDGTSTAVSTRIINENLKIYLDNLIKYNKIKDKVDFKEADILQENKLNLSDFFNVESYAKYSLQKGIDYYNEILGGKTLKNGTKLKGLNEVINEYKQKNKSGELSKFKMLKKQILGEGEDRTLFEEIENEDELKDVLKDFFYNADPKITLFKTLLEDFFSNTEKYKDELDKIYFNTVAINGILHRWVDDSGVFQKYLFEVLKSNKLVKSNHYDKKEDSYKFPDFISFEHIKVALENCERDGLKDKFWKEKYYTKECLTENGLANLWQEFLEIYKCEFKKLYDYKTDDNDCYLQYRDNYKKYILDANFNPKEKSAKDIIKDYLDSVLSIYQLAKYFALEKKKVWTTDYETGDFYYEYIKFYEDTYEQIIKPYNLVRNYLTRKPINTAKKWKLNFDNAYLASGWDKDKEVSNLTVILRRDEQYYLAIMKKGKNKIFEKKFSCGEFEKMEYKQIAEASSDIHNLVLMNDGSCRRCIKMHDKRKYWPLDISIIKEKKSYAKENFVRRDFERFVNYMKKCSLLYWKEYDLKFSDTSTYKNINDFTNEIASQGYKLSFSAIPESYINEKNNNGELYLFQIYNKDFGIKTEGNKNLHTMYWESIFSEENRFRNFIVKLNGKAEIFYRPKSEQVEKEQRNFTREIIKNRRYTENKIYFHCPITLNRISRENVKKFNNGINNYIATNPNINILGVDRGEKHLVYYAIVDQDGKLIDAEDATGSFNTIGSTDYHRLLEEKAKDREKERKDWDLIRGIKDLKKGYISLVVRKIADLAIKYNAIIIFEDLNTRFKQIRGGMEKSVYQQLEKALINKLSFLVNKGEKDPEQAGHLLKAYQLAAPFQTFDKMGRQTGIIFYTQASYTSKIDPITGWRPNLYLKYRNIDDSKESIKKFKSILFNKEKNRFEFTYDLKDFVDFEEDKIPEKTEWTLCSSVERHKWNRHMNNNKGGYEVYKDLTENFYKLFDENNISMNKDIVDQVESISNGNFFRQFIYLFNLVCQIRNTDEKAEDVDKRDFILSPVEPFFDSRRAKDFKAYGDNLPKNGDENGAYNIARKGVLIIKKIKEYYNQNGSCDKLGWGDLSISHKEWDDFATNN

SEQ ID NO: 276 - protein, 1301 aa, unidentified; identical to SEQ ID NO: 11
  source 1..1301: note = Description of Unknown: Cas12a sequence

SEQ ID NO: 277 - protein, 1188 aa, unidentified
  source 1..1188: note = Description of Unknown: Cas12b sequence
MCVSRLPWFNITLTGKLNRQRLNQMCVSRLPWFCTPKGQLAATPKTVVAQQENAMLAIIRDVHEAAPADLKTVAQRLEPGYFVTQFPKQQMTGDEARAEAERLFAACQKKFKELAEYEDGYRQCLDALGPNLSLPRLGRKPKGAYPYAVVFKLMPTNATWECFKRVTASLYKRAQKGVVSPVSADSIADVRTNDEPLFEYFTNLALVRPPGNKDRAVWFEFDLAAFIEAIKSPHQFFQDTIKREQAVAQIKAKLDAMDGQGRAASGEEDALPGFEGDDRITLLRELVTDTLGYLAEADASTSPGGKIEYSIQERTVRGFAEVKRRWRDLVEKGKATEDALLKVLAEEQTEHRDDFGSATLYRELAKPKFQPIWRDPGTQPWHADDPLRAWLEYRELGRELEDKQRPIRFTPVHPVHSPRFFIFPKKKGGGRFGTVHEPGQLRVMAGIVAQTQHGWEPVPVRITYAAPRLRRDQLRDDVETDLESRPWLQPMMQALGLPEPDTADFSNCRVTLQPSAPDDIQLTFPVDVSADKLTTAIGKAARWAKQFNLFPDGDNFYNASLRWPHEKKPSKPPVPWHEALDNFSVLAADLGQRCAGAFARLEVRANDDFAGKPSRFIGETPGKKWRAALVAAGMLRLPGEEQTVWRPGATGPNFHTELSGSRGRMARPHEADDTADLLRAFDCPEESLMPADWRTSLSFPEQNDKLLVAARRYQSRLARLHRWCWFLTDEKKRQTALDEIREAEDMPAADDPQLTDKLRALLLQKQAALPGLLVRLANRILPLRGRSWQWETHPDKADCHLLTQTGPALPDVWIRGQRGLSMQRIEQIEELRRRFQSLNQMQRREIGGKPPIRRDDSIPDCCPDLLDKLDQIKEQRANQAAHMILAEALGLRLAPPPADKRQLRASRDVHGQYVKSREPVDFIVIEDLSRYRSSQGRAPRENSRLMKWCHRAVRDKLRELCEPFGIPVVETPAAYSSRFCSRSGVAGFRAVEVGPGFDREFPWMMLKDREDEGEPVRQLILQVATLNQGRDGKPPRTLLAPLAGGPIFVPIVDKLNGADIQPALAQADINAAINLGLRAIADPRLWSIHPRCRTQRQGDQMLTREKRKFGETGQPLAVHRADGVKPDDTRNPNFFADISGSLPAWESATLDGQHLLSGRCLRSEIKKRQWQRCAEINDRRMNRWMKGE

SEQ ID NO: 278 - protein, 1518 aa, unidentified
  source 1..1518: note = Description of Unknown: Cas12b sequence
MTELQTQRAYTLRLKGIDEKDQSWRDALWKTHEAVNKGAKVFGDWLLTLRGGLDHTLADAEIPGEKGKPDRAPTQEERKHRRILLALSWLSVESERGAPEEFIVATGKEPAATRNDKVIAALKDILRGRNLTEEKISEWTEVCTPSLSAAIREDAVWVNRSRAFDEAVKRIGSSLTREEVWDMLECFFGSRNAYLAPVKISEDESSDGEQEEKAKDLVQKAGQWLSSRFGTGEGADFAKMAAVYAKIAAWAGNAQAGTTGNEVINNLATALREFTPKSNDLKGVLDLISGPGYKSATRNLLKQIANTKTVTREDISKLQETAGEDSEECATKTGSKGKRAYADAILKDVESVCGFTYRIDKDGQPVSVADYSKYDEDYKWGSSRHKEFAVMLDHAARRVSLAHTWIKRAEAERRKFEEDSKKIMQVPQAAKDWLDAYCAQRSEASGALEPYRIRKRAIQGWKEIIASWNKPDCKTAEDRIAAARQLQDDPEIEKFGDIQLFEALAEDDAQCVWKKEDGTLDPEILINYTLASEAMFKKQHFKVPSYRHPDAFLYPVFCDFGNSRWELDFSIREAATKLKEIEAKIEKQRQEVHKVQQALEKCENDEKRPKMEERLKEAQKKLQESQNYGEYLHSNNKITMVLFDGTFVKKHIFAWQSKRLTKDLALYQEPSADPKNVVSRADRLGRAVASVGINDAVKVAGLFEQENWNGRLQAPRQQLEAIAQYVEKHGWDNKAEKMRASIKWFITFSAKLQSKGPWNEFARKHGLKEDPHYWPHAEKNENRTAHSRLILSRLPGLRVLSVDLGHRYAAACAVWEALGSEAFKKDIEGKRIIRGDTDENALYCHTEHEANGKKHITIYRRIGADTLPDGAHHPAPWARLDRQFLIKLQGEDEQAREASNEEIWKVHQLENTLGRRTPLIDRLIAGGWGYTEKQKARLEVLTNLGWCPTNKTDNQEEGDEEETAILSKPSLLVDDLMFSAVRTLRLALKRHGDRARIAHYLITDEKTKPGGVKEKLDKNGRVELLLDALGLWHDLFSSPGWHDEKAKQLWNAYIAGLLPEGELQQAKSVTTSAALGGQQKKEKKEKLRAVAEALYLNSDLCHSLNEVWRKRWEEDDKQWRIYIRWFKDWIMPRGANAKSPAIRHVGGLSLTRLATLTEFRRKVQVGFFTRLHPDGTKTETREDFGQKTLDTLEHLREQRVKQLASRIVEAALGIGSEDKRHWDGKKRPRQRIADPRFVPCHAVVIENLTHYRPEETRTRRENRQIMEWASSKVKKYLSEICQLHGLHLREVSAAYTSHQDSRTGAPGIRCQDVSLIEFMKSPFWRKQVAQAEKKQKEGKGDAVERYLCELNQKWKGASEEEWRKAGFVRIPLRGGEIFVSAAGHDSPAAKGIHADLNAAANIGLRALLDPDWSGKWWYVPCNSSTMCPARDKVTGSAAVNPGQPLQVSAQLESDDAAKDTKKRKKKGDGKSKEIINLWRDISSYPLEDTRGGTWSNKTVYWNRVQSNVVHILQNQMKG

SEQ ID NO: 279 - protein, 1374 aa, unidentified
  source 1..1374: note = Description of Unknown: Cas12b sequence
MPETTQRAYTLRLQGHDPKDASWREALWKTHEAVNRGAKAFGDWLLTLRGGLDHSLADEGAPGQTPTEEQRKQRRILLALSWLSVESENGAPQEYIVPHDRDNESGARQNWKTREALREILKNRGCRDDEIESWCHDCEPSLTSAIRKDAVWVNRSKAFDNAVQSIPNFSREEIWDLLGCFFVSSQAYLAPLESPKDDKPDASKKDSSKDLIQSAGQWLSRRFGRGKGLNFARLAETYEAIARWASVANPGDTNDLIADLAKTLNAETPELDGILKVVSGPGHKSKTRNLLRSLSAVNHITKDTLQRLKDTANEDAKKAKLKKGEKGHRAYAYKVLEAVEDACGFTYLQEGDRAKHCEFAVMLDHAARRVSSLHTWIKRAEAERRRFEIDTKKKDQLPPSVKEWLDTYCQKRSKETGAVEPYRIRRGAIEGWKEIVEAWSKAGTTTAEDRKHEARRLPDNPHIDKSGDIKLFEDLALEDALPVWHANGDPNNPPDPQLLIDYVEGSEAEFKKRAFKVPTYCHPDPLVHPVFCDYGCSRWNVSFAIQPVKKQKLSSEEKLPAKGLLLDLLHGTAIRPVALRWQSKRFARDLALNTTDSSDKPNEVTRADRFGCALAKCPSSQKIRIRGLFEEKYWNGRLQAPRPELTALAKRVAKYGWDKKARKLRNSLNWFITFSANLRPSGPWEEYTKYAEKAFSSNASAKPSVSRGGFWVVHASPNKRGKMAQLRLCRLPELRVLSVDLGHRYAAACAVWETLSKSAFEQEIHERKILRGGTGPNDLFCHTQHDTNGQSKVTIYRRIGADTLPNGTPHPAPWARLDRQFLIKLPGEEREARKASPTELANVEKLEKELGLKTSENRVKRIDDLMSDTLRTVRQALRRHSLRARIAFNLATLRDQSDGDEESQSKQKRDTRWNNTVKIWHSLLESNEWTDDWAKALWDELGPLSDPQKADDAEWLKLAAEKFYTRWQEDEQTWRERLRWLRRWILPRGSQAASQKGSIRHVGGLSLTRLATIKTLYQVLKAYHMRLKPDNSRKNIPAEGDEALQNFGQKILDDLEHMREQRVKQLASRIVEAALGLGRMKQVTIGKDPKRPREPVDQSCHAVVIENLTHYRPEKRQTRRENRQLMDWSAAKVKKYLKECCQLHGLHLVEVSASYTSRQDSRTGAPGIRCQEVPLTDFLKKNFWREQVKQAKQRLSEGKANARDRYLCQLNERWGNAPAPVTQTAIRLRIPLNGGELFVSADQNSPASKGIQADLNAAANIGLRAITDPDWPGAWWYVPCEANTFKPVKDKVAGSAAIDSNVSLKKDSPNSEKPASDRKSRTSKSMINLWCDTSSKSLSEKDQWQESAPYWEDVAARTINILQASLACSTTNSQ

SEQ ID NO: 280 - protein, 1335 aa, unidentified; identical to SEQ ID NO: 274
  source 1..1335: note = Description of Unknown: Cas12a sequence

SEQ ID NO: 281 - protein, 1520 aa, unidentified
  source 1..1520: note = Description of Unknown: Cas12b sequence
MAYQNGKEQPTVTNQRAYTLRLSGTNDQDSIWRNRLWHTHEAVNKGAKTFGDWLLTMRGGLCHTLAEADVPGKGNKPARHPTPQEIRSRRVVLALSWLSVESQHGAPERHLVSHDLDIATGERKNWKTVEALREILHGRCLCKELIDEWANDCRDSLSATIREDAVWVNRSKAFDLAAKKIGASLTREELWDFLQPFFANKHGYLQMDTVAGVTNGDSETDAEEAKEDSSEEKAKDLSQKAGQWLSSRFGTGTGADFSRFSKVYEVLAARCGSVAVGVSGVEAIRILAGTLADFSPGSNDIEGMLGLMSGPGYKSATRNILQKINTLQTVSQQDLDRLREASEKDALQSKQKVGGKGSRPYANAILQDVEAACGICYAGTGESPARHWQYAVILDHAARRVSMAHSWIKRAEEQRSKFEIEKDKLDHVPKDALAWLDAFCARRSSESGASDAYRIRRSAVDGWKQVVAAWAALPPKPENQGSELLSDAESARIQAARELQDTVEKFGDIQLFEALSLTGAKCVWQPDGRPDAQPLLDYVAGTDAISKKQRFKVPAYRHPDALLHPVFCDFGNSRWNINYAIHRAPEKLTPAQQLLEKKKAEIDKAELTLAKAGDAAKQANISEKINGLRAAFIQQQEKVAWLNSRHAMTMSLWDGTHIEDTPLIWQSKRFGSDIGQPVEAQPLPVSRADRFGRAVALAQDNVPVIPSGLFDLSDWNGRLQAPRRQLEAIAAIRDSAKLSVNEKQQLVAKRIQSIRWLLTFSAKLQSHGPFIAYAAQHGFDWRYGAHGPENKSRQGLAKLILCRLPGLRILSVDLGHRYAAACAVWETLNAGQIQKACLDAGKEAPGPCTLYLHLKQIANGKEKKTIFRRIAADTLPDGSPHPAPWARLDRQFLIKLQGEDRDARLATSEEIAAVEQMENELGVVRQLKRKGRELLVDELMSDALRTLRLGLRRHGVRARIAFNLTANKRIRPGGKEEVLDQEGRVLLLTETLLAWYELYTAERWTDEPARELWNRHIQPLLGATILQNTVNQEDTPSAAKRRKLREETSGKLKHVAEEIAKNDSLCRQLHVLWSAQWQTEDVIWRTRLRMMRRWLLPRGVKRNAQLRISIRDVGGLSLTRIASFKSLYQVQKAYQMRPHPEDPRLNIPERGDSRLENFGQRVLDAMERMRENRVKQLASRIAEAALGIGGETGISSKDGSQKKRPTERSSDPRFAPCHAVVIEDLTHYRPDETQTRRENRQLMSWSSSKVKKYLGEACELNGLYLREVSPAYTSRQDSRTGAPGLRCNDVTVVEFNNSPFWRKQVGAAEKNQKEGNKGDARERYLLSIEEGIRGAANDRDIFRIPVKGGEIFVSACITDGGNNAKKNAPPGLQADLNAAANIGLRAIFDPDWEGRWWYIPCDAATLCPDAKKFIGCKAVDPTKPLRVVAEEGAISASGIGSKKSGRKKNAATDGTRIVNLWRDPSGAPIHRDVLRSPEWQDYAGYWNEVQHRVIRNLKTCYEQTSQQEDPFVSQDADKPF

SEQ ID NO: 282 - protein, 1180 aa, unidentified
  source 1..1180: note = Description of Unknown: Cas12b sequence
MKRLAETALADKVKCETNSRPKGERAYANSILHDVESACGFTYRVDKGEQPVPVSDYSHYANDYRWGPANHSEFAVMLDHAARRVSLAHTWIKRAEAERRQFEENAKKIDKVPKVAREWLDSLCAERSIVLGALEPYRIRRRAVDGWKHVVAAWSKSDCKTAQDRITAARLLQEDPEIDKFGDIQLFEALAEDHAVCVWQRDGEAGKTSDPQLLIDYALAAEAEFKKRHFKVPAYRHPEAFWHPVFCDFGQSRWKICFDVHKNRQSRRQRACANRISRKICFDVHKKRQTLRLSLEVWTGSKMLDMPLCWQCKRLARDLALGQDHKKDRSCQVTRADRLGRAVSNVARNQEVQILGLFEQEYWNGRLQAPRPQLEALGRYIEKHGWDAKAQKSCRAIRWMISFSPRLQPAGPWGKFAEKLQLNPNPKYWPHAEDNKDRGSRSKLILCRLPGLRVLSVDLGHRYAAACAVWEAVDAEQVKEACQAAGHREPNENDLYLHLKKRTTKQKKGSQGVVEETTIYRRIGADTLPDCTPHPAPWARLDRQFLIRLQGEEDEARAASNEEVWAVHKLEAELGRTIPLIDRLLGAGWGQTEKQKARLKALRELGWTPANKCQAFNSTDETELRRPSLAVDELMLDAVGTLRLALKRHGDRARIARYLITDERTKPGGVKEKLDENGRIELLQDALIIWHGLFSSPRWRDDAAKQLWNEHIAKLVGEQNLVEVSEDASGSERRTKQKQNREKLREAAKALVDDVALRQALHDMWKRRWEEEDREWRRRLRWFKDWVLPRREQARKAYSRPAETGSSSHPKRRARYAAIRRVGGLSLTRLATLTEFRRKVQVGFFTRLKPDGTKAEAKEGFGQSTLDALEHLRAQRVKQLASRIVEAALGVGRIRRFPGVKNPKRPDTPVDKPCHAIVIENLTHYRPEETRTRRENRQLMTWSSSKIKKYLAEACQLYGLHLREVTAAYTSRQDSRTGAPGLRCQDVPVKEFMRSLFWRKEVAQAEKKLTAGKGSSYERLLCELNQRWKDNSPGDGKRAELLRLPHKGGEIFVSAAPDSPAARGLQADLNAAANIGLRALTDPDWPGKWWHVPCNAVTFRPVEDKVKGSAAVKLDQSLRQVAHPQSKDPGAKKSKEIVNLWCDISSLPLEHREWKLDWEPYPAYWNNVQCRVIRVLQGKV

SEQ ID NO: 283 - protein, 1546 aa, unidentified
  source 1..1546: note = Description of Unknown: Cas12b sequence
MANAKVKTTTRSYTLSLNAPSDTTDRSPLWHRIFRTHYAICCGAREFGKLLLDLRGGLPTSLAQLGEGIAENDRRQTQRGTRRILALGWLSVEDLDHARNDPHRVQDTAPGSPLDQDLAEKILRKILITKGIKSEEEQSNWISDCLPALTANIRPDAVWVNRAESFAQWQRGTQPGAQPPTPEEAQQILFSLCGESLVTLTLPEQPAAAGQKQPDQETSPDPEEQTDRPPAAPSADDEMDPSNASRGIFGDLFGENAEGKRSRSQGKDNFACAVRDFLCANPTPSADAITEFREKQKPREPNPPGPEKYPPEVSTSGAPTAVAKRYRKLLVCAGLWPKSADEDGSSRNSAKTKFADPKEPQKTEIQINALDLIDACNQAAPADDSGTSPKAGRVFAPAWASNIAEKVASATQMPANAKSLNEFKRLMFALAARRFSQTQSWTRRNEAERHMAAARQDAAVARLREIDPDHKAQDWLRGYEQRRADQSGSNGEFRITRRMIGEAEAVFKAWAGTNSAAERELKTVAVQTTAEKFGDAALYSEIARNTAAEAVWRSGSAPEILDQWVKLRKAQSDQQRTRVPRFCHPNAFRHPTWCEFGESSKPGVWYAWNPKSKPRKPEVGGEGDGTRRLWVLLPDFNSGIGQAVPLRWRSKRLSKDLGEALQPSDAPIPRADRVSIAAAGLNLEGANGVPARYRPSLPFSENTKGWNARLQANRTALLHLESKWDAEAATWRDGGRSLLALKWFTTFSPELAMSEGPGRAIHPKLGWNSEPHSDLNRAQKRGGNAKLILSRLPGLRVLSVDLGHRYAAACAVWETLTTEQMNAACQAKNHTQPAESDMYVHLAHPTERVVKSGRKKGQNLIQTTVYRRIAADTLPDGTPHPAPWGRLDRQFLIKLQGEQRPTRAASKNEADLANALFHRLGLRSDADSENKSRAVDKLMARTVRVATLGLKRHARRAKIAYALDPNTKAIPGMGGSSAAFTPGDEPHIHLLTDALFDWQSLATDAKWDDAHARSLWNHHIATLPGGFHLENPTPRDESAHEPSRQRQRSGDDALRATLKPIAEKLSKADRQEVHAAWKKYWGDSDGQSAIVPKVLQGQRGPEKTTPSASASGWHGKIRWITDWIMGKYLEGCTGHAWKHDVGGLSVSRITTMKSLYQLHKAFAMRATPEKPRGAPEKGESNLGAAQGILTAMESMRQQRVKQLASRIAEAALGAGIERRSDNGRELQRPRERVDDPRFAACHAVVVEDLTNYRPDEMQTRRENRQLMQWASSKVKKYLSEACQLHGLYLRGVPAGYTSRQDSRTGAPGVRCGDIPVEELMAAPRWRRQILTAEKTRRENNTGTARDRYILTLDEKYRLLTAEQRKKTPPARIPVKGGDLFVSADPDSPAASGIQADLNAAANIGLKALIDPDWPGRWWYIPCDATTHKPSPERTRGSAAVDCDVPLGPDSTGTPEDRDAKPKKNQRNSKIAGRGQSAIINLWRDPTHLPIKENPSAWCESKKYWNQVEHNVVKVIESKGQKLTQTAEAATGESASSPPIAPTDVPW

SEQ ID NO: 284 - DNA, 91 bp, synthetic construct
  source 1..91: mol_type = other DNA; note = Description of Artificial Sequence: Synthetic oligonucleotide
agtgtctttgcaggaaagaacacagatcttgagggtcacaactcccatgtaggcggagactgcaacccctatagtgagtcgtattaatttc

SEQ ID NO: 285 - DNA, 91 bp, synthetic construct
  source 1..91: mol_type = other DNA; note = Description of Artificial Sequence: Synthetic oligonucleotide
agtgtctttgcaggaaagaacacagatcttgagggttgcagtctccgcctacatgggagttgtgacccctatagtgagtcgtattaatttc

SEQ ID NO: 286 - DNA, 91 bp, synthetic construct
  source 1..91: mol_type = other DNA; note = Description of Artificial Sequence: Synthetic oligonucleotide
tctttgcaggaaagaacacagatcttgaggggtgtagttcccctcaatttggggatgaacgtcgacccctatagtgagtcgtattaatttc

SEQ ID NO: 287 - DNA, 91 bp, synthetic construct
  source 1..91: mol_type = other DNA; note = Description of Artificial Sequence: Synthetic oligonucleotide
tctttgcaggaaagaacacagatcttgagggtcgacgttcatccccaaattgaggggaactacaccccctatagtgagtcgtattaatttc

SEQ ID NO: 288 - DNA, 136 bp, synthetic construct
  source 1..136: mol_type = other DNA; note = Description of Artificial Sequence: Synthetic polynucleotide
ttgtgagcggataaacacaggtgccacttctcagatttgagaagctcaacgggctttgccacctggaaagtggccattggcacacccgttgaaaaattctgtcctctagacccctatagtgagtcgtattaatttc

SEQ ID NO: 289 - DNA, 82 bp, synthetic construct
  source 1..82: mol_type = other DNA; note = Description of Artificial Sequence: Synthetic oligonucleotide
ttccggctcgtatgttgtgtggaattgtgagcggagtgccacttctcagaccgctcgccctatagtgagtcgtattaatttc

SEQ ID NO: 290 - DNA, 113 bp, synthetic construct
  source 1..113: mol_type = other DNA; note = Description of Artificial Sequence: Synthetic polynucleotide
cgagcggtcatcttgaagccaacggggtgtttgctcttggaaagagcacattggcacttcccgttgtcctcgccgtcctatagacgacccctatagtgagtcgtattaatttc

SEQ ID NO: 291 - DNA, 60 bp, synthetic construct
  source 1..60: mol_type = other DNA; note = Description of Artificial Sequence: Synthetic oligonucleotide
aattgtgagcggataaacacaggtgctaatgcctcccctatagtgagtcgtattaatttc

SEQ ID NO: 292 - DNA, 105 bp, synthetic construct
  source 1..105: mol_type = other DNA; note = Description of Artificial Sequence: Synthetic polynucleotide
gagacatcgtccagcaataggagtttctcacaccctgcagcacttatagctagacggttgtcctgaccaaaagacagaacccctatagtgagtcgtattaatttc

SEQ ID NO: 293 - DNA, 84 bp, synthetic construct
  source 1..84: mol_type = other DNA; note = Description of Artificial Sequence: Synthetic oligonucleotide
atggtcatagctgtttcctgtgtttatccgctcagtgctaatcacatttaattcatctaccctatagtgagtcgtattaatttc

SEQ ID NO: 294 - DNA, 105 bp, synthetic construct
  source 1..105: mol_type = other DNA; note = Description of Artificial Sequence: Synthetic polynucleotide
gataaataatgtaatcctgtggttgaatggattttttccatccttagcacacgcacagtattctttgccctttaggcaaaccctatagtgagtcgtattaatttc